(19) United States
(12) Patent Application Publication
(10) Pub. No.: US 2026/0263501 A1
(43) Pub. Date: Sep. 10, 2026

(54) COMPOSITIONS AND METHODS FOR ALTERING COMPLEMENT ACTIVATION

(71) Applicants: Beam Therapeutics Inc., Cambridge, MA (US); Apellis Pharmaceuticals, Inc., Waltham, MA (US)

(72) Inventors: Nageswara Kollu, Waltham, MA (US); Tara Barbour, Waltham, MA (US); Pascal Deschatelets, Waltham, MA (US); Lei Johnson, Cambridge, MA (US); Tanggis Bohnuud, Cambridge, MA (US); Lo-I Cheng, Cambridge, MA (US); Michael Packer, Cambridge, MA (US); Brian Cafferty, Cambridge, MA (US)

(73) Assignees: Beam Therapeutics Inc., Cambridge, MA (US); Apellis Pharmaceuticals, Inc., Waltham, MA (US)

(21) Appl. No.: 19/679,108

(22) Filed: May 15, 2026

Related U.S. Application Data
(63) Continuation of application No. PCT/US2024/056761, filed on Nov. 20, 2024.
(60) Provisional application No. 63/601,145, filed on Nov. 20, 2023.

(51) Int. Cl. A61K 31/7105; A61K 38/46; A61K 38/50

(52) U.S. Cl. CPC A61K 31/7105; A61K 38/465; A61K 38/50; C12Y 305/04004

(57) ABSTRACT

Compositions and methods for reducing complement activation by introducing one or more alterations into a complement factor B (CFB) polynucleotide in a cell. In particular embodiments, the invention of the disclosure features a base editor system (e.g., a fusion protein or complex comprising a programmable DNA binding protein, a nucleobase editor, and gRNA) for modifying a CFB polynucleotide, where the modification is associated with reduced expression, and/or reduced activity of the CFB polypeptide encoded by the polynucleotide.

### CROSS REFERENCE TO RELATED APPLICATIONS

This application is a continuation under 35 U.S.C. § 111 (a) of PCT International Patent Application No. PCT/US2024/056761, filed Nov. 20, 2024, designating the United States and published in English, which claims priority to and the benefit of U.S. Provisional Application No. 63/601,145, filed Nov. 20, 2023, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on Nov. 20, 2024, is named 180802-055803PCT_SL.xml and is 4,134,393 bytes in size.

### BACKGROUND

The complement system is an important part of the innate immune system and is involved in the clearance of microbes and cellular debris, as well as the activation of inflammation and diverse immune pathways. Overactivation of the complement system or inappropriate targeting to one's own cells can lead to disease; however, inhibition of complement system activity has been successfully and safely shown to provide therapeutic benefit for patients suffering from an overactive complement system. Therefore, improved methods for reducing complement system activation in such patients are of interest.

### SUMMARY

As described below, the present disclosure features compositions and methods for reducing complement activation by introducing one or more alterations into a complement factor B (CFB) polynucleotide in a cell. In particular embodiments, the invention of the disclosure features a base editor system (e.g., a fusion protein or complex comprising a programmable DNA binding protein, a nucleobase editor, and gRNA) for modifying a CFB polynucleotide, where the modification is associated with reduced expression, and/or reduced activity of the factor B polypeptide encoded by the polynucleotide. Non-limiting examples of alterations include base edits.

In another aspect, the disclosure provides a method of treating a disease or disorder associated with inappropriate activation of the complement system in a subject in need thereof. The method involves altering a nucleobase of a complement factor B (CFB) polynucleotide in the subject by administering to the subject one or more guide polynucleotides, or one or more polynucleotides encoding the one or more guide polynucleotides, and a base editor containing a nucleic acid programmable DNA binding protein (napDNAbp) domain and a deaminase domain, or one or more polynucleotides encoding the base editor. The method involves (a), (b), (c), and/or (d), where in (a) the one or more guide polynucleotides targets the base editor to effect an alteration of a nucleobase of the CFB polynucleotide that: i. disrupts a splice site in the CFB polynucleotide, ii. alters a start codon in the CFB polynucleotide, iii. alters a TATA box in the CFB polynucleotide, iv. introduces a new stop codon in the CFB polynucleotide, and/or v. alters a nucleobase in a codon encoding an amino acid residue within a region of the CFB polypeptide encoded by the CFB polynucleotide selected from one or more of: serine protease (SP) active site, Mg²⁺ binding loop, cleavage site, salt bridge, and oxyanion-hole. In (b) the deaminase domain contains a TadA variant (TadA*) containing an amino acid sequence having at least 90% sequence identity to the following TadA*7.10 amino acid sequence: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), or a fragment thereof lacking only the N-terminal methionine, where the TadA* further contains a combination of amino acid alterations compared to the TadA*7.10 amino acid sequence selected from one or more of: i. Y123H, Y147R, and Q154R, ii. I76Y, Y133H, Y147R, and Q154R, iii. V82S, and Q164R, iv. I76Y, V82S, Y123H, Y147R, and Q154R, v. I76Y, V82T, Y123H, Y147R, and Q154R, and vi. I76Y, V82T, Y123H, Y147T, and Q154S. In (c) the one or more guide polynucleotides contain a nucleic acid sequence containing at least 10-23 contiguous nucleotides of a spacer nucleic acid sequence selected from CCUCAGAUGUCUAUGUGUUU (SEQ ID NO: 1524), UGCUUACAAUGACUGAGAUCU (SEQ ID NO: 1535), UUGCUCCCCAUGGCGUUGGA (SEQ ID NO: 3476), CCCCAUGGCGUUGGAAGGCA (SEQ ID NO: 3443), and UGCUCCCCAUGGCGUUGGAA (SEQ ID NO: 3467) and/or listed in any one of Tables 2A to 2H. In (d) the one or more guide polynucleotides targets the base editor to effect an alteration of a nucleobase in one or more codons encoding an amino acid residue selected from one or more of amino acid residue 1, 171, 175, 176, 177, 202, 203, 229, 230, 231, 232, 233, 254, 255, 256, 257, 258, 259, 260, 275, 276, 277, 278, 279, 280, 281, 351, 353, 354, 389, 470, 471, 472, 525, 526, 529, 574, 575, 576, 696, 697, and 699 relative to the following complement factor B reference amino acid sequence: MGSNLSPQLCLMPFILGLLSGGVTTTPWSLAQPQGSCSLEGVEIKGGSFRLLQEGQALEYVC PSGFYPYPVQTRTCRSTGSWSTLKTQDQKTVRKAECRAIHCPRPHDFENGEYWPRSPYYNVS DEISFHCYDGYTLRGSANRTCQVNGRWSGQTAICDNGAGYCSNPGIPIGTRKVGSQYRLEDS VTYHCSRGLTLRGSQRRTCQEGGSWSGTEPSCQDSFMYDTPQEVAEAFLSSLTETIEGVDAE DGHGPGEQQKRKIVLDPSGSMNIYLVLDGSDSIGASNFTGAKKCLVNLIEKVASYGVKPRYG LVTYATYPKIWVKVSEADSSNADWVTKQLNEINYEDHKLKSGTNTKKALQAVYSMMSWPDDV PPEGWNRTRHVI ILMTDGLHNMGGDPITVIDEIRDLLYIGKDRKNPREDYLDVYVFGVGPLV NQVNINALASKKDNEQHVFKVKDMENLEDVFYQMIDESQSLSLCGMVWEHRKGTDYHKQPWQ AKISVIRPSKGHESCMGAVVSEYFVLTAAHCFTVDDKEHSIKVSVGGEKRDLEIEVVLFHPN YNINGKKEAGIPEFYDYDVALIKLKNKLKYGQTIRPI CLPCTEGTTRALRLPPTTTCQQQKE ELLPAQDIKALFVSEEEKKLTRKEVYIKNGDKKGSCERDAQYAPGYDKVKDISEVVTPRFLC TGGVSPYADPNTCRGDSGGPLIVHKRSRFIQVGVISWGVVDVCKNQKROKOVPAHARDFHIN LFQVLPWLKEKLQDEDLGFL (SEQ ID NO: 426), or a corresponding position in another CFB polypeptide sequence. The method results in altering the nucleobase of the CFB polynucleotide.

In another aspect, the disclosure provides a method of treating a disease or disorder associated with inappropriate activation of the complement system in a subject in need thereof. The method involves altering a nucleobase of a complement factor B (CFB) polynucleotide in the subject by administering to the subject one or more guide polynucleotides, or one or more polynucleotides encoding the guide polynucleotides, and a base editor containing a nucleic acid programmable DNA binding protein (napDNAbp) domain and a deaminase domain, or one or more polynucleotides encoding the base editor. The method involves (a) and (b), where in (a) the deaminase domain contains a cytidine deaminase or a TadA variant (TadA*) containing an amino acid sequence having at least 90% sequence identity to the following TadA*7.10 amino acid sequence: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), or a fragment thereof lacking only the N-terminal methionine, where the TadA* further contains a combination of amino acid alterations compared to the TadA*7.10 amino acid sequence selected from one or more of: i. Y123H, Y147R, and Q154R, ii. I76Y, Y133H, Y147R, and Q154R, iii. V82S, and Q164R, iv. I76Y, V82S, Y123H, Y147R, and Q154R, v. I76Y, V82T, Y123H, Y147R, and Q154R, and vi. I76Y, V82T, Y123H, Y147T, and Q154S. In (b) the one or more guide polynucleotides contain a spacer containing a nucleotide sequence selected from one or more of: AGGUGAUUCUGGCGGCCCCU (SEQ ID NO: 1719; gRNA1536), CGCCAGAAUCACCUGCAAGG (SEQ ID NO: 1715; gRNA1532), CUAUGACGUUGCCCUGAUCA (SEQ ID NO: 1723; gRNA1540), UGCUCCCCAUGGCGUUGGAA (SEQ ID NO: 3467; gRNA3657), UUGCUCCCCAUGGCGUUGGA (SEQ ID NO: 3476; gRNA3658), CCCCAUGGCGUUGGAAGGCA (SEQ ID NO: 3443; gRNA3660), CCUCAGAUGUCUAUGUGUUU (SEQ ID NO: 1524; TSBTx3826), GCUUACAAUGACUGAGAUCU (SEQ ID NO: 1534; TSBTx3837), UGCUUACAAUGACUGAGAUCU (SEQ ID NO: 1535; TSBTx3837), and UCUCACCUCUGCAAGUAUUG (SEQ ID NO: 1529; TSBTx3835). The method results in treating the disease or disorder associated with inappropriate activation of the complement system in the subject.

In any aspect or embodiment of the disclosure, the splice site is located near the 3′ end of Exon 1, Exon 10, Exon 11, Exon 12, Exon 14, Exon 15, or Exon 16 of the CFB polynucleotide. In any aspect or embodiment of the disclosure, the splice site is located near the 5′ end of Exon 5, Exon 8, Exon 9, Exon 10, Exon 11, Exon 14, or Exon 18 or the CFB polynucleotide.

In any aspect or embodiment of the disclosure, the one or more guide polynucleotides target the base editor to effect an alteration of a nucleobase in a codon encoding an amino acid residue selected from one or more of M1, P171, V177, R203, E232, E255, K258, R259, K260, D276, S278, S280, T353, D389, E471, H526, Y575, D576, G697, and S699 relative to the following complement factor B reference amino acid sequence:

(SEQ ID NO: 426)
MGSNLSPQLCLMPFILGLLSGGVTTTPWSLAQPQGSCSLEGVEIKGGSFR
LLQEGQALEYVCPSGFYPYPVQTRTCRSTGSWSTLKTQDQKTVRKAECRA
IHCPRPHDFENGEYWPRSPYYNVSDEISFHCYDGYTLRGSANRTCQVNGR
WSGQTAICDNGAGYCSNPGIPIGTRKVGSQYRLEDSVTYHCSRGLTLRGS
QRRTCQEGGSWSGTEPSCQDSFMYDTPQEVAEAFLSSLTETIEGVDAEDG
HGPGEQQKRKIVLDPSGSMNIYLVLDGSDSIGASNFTGAKKCLVNLIEKV
ASYGVKPRYGLVTYATYPKIWVKVSEADSSNADWVTKQLNEINYEDHKLK
SGTNTKKALQAVYSMMSWPDDVPPEGWNRTRHVIILMTDGLHNMGGDPIT
VIDEIRDLLYIGKDRKNPREDYLDVYVFGVGPLVNQVNINALASKKDNEQ
HVFKVKDMENLEDVFYQMIDESQSLSLCGMVWEHRKGTDYHKQPWQAKIS
VIRPSKGHESCMGAVVSEYFVLTAAHCFTVDDKEHSIKVSVGGEKRDLEI
EVVLFHPNYNINGKKEAGIPEFYDYDVALIKLKNKLKYGQTIRPICLPCT
EGTTRALRLPPTTTCQQQKEELLPAQDIKALFVSEEEKKLTRKEVYIKNG
DKKGSCERDAQYAPGYDKVKDISEVVTPRFLCTGGVSPYADPNTCRGDSG
GPLIVHKRSRFIQVGVISWGVVDVCKNQKRQKQVPAHARDFHINLFQVLP
WLKEKLQDEDLGFL.

In any aspect or embodiment of the disclosure, the one or more guide polynucleotides contain a spacer complementary to both a human CFB polynucleotide and a non-human primate CFB polynucleotide. In any aspect or embodiment of the disclosure, the one or more guide polynucleotides contains a spacer complementary to a human CFB polynucleotide but not to a non-human primate CFB polynucleotide. In any aspect or embodiment of the disclosure, the one or more guide polynucleotides contain a spacer containing only 20 or 21 nucleotides. In any aspect or embodiment of the disclosure, the one or more guide polynucleotides contain a spacer containing a nucleotide sequence selected from one or more of: AGGUGAUUCUGGCGGCCCCU (SEQ ID NO: 1719; gRNA1536), CGCCAGAAUCACCUGCAAGG (SEQ ID NO: 1715; gRNA1532), CUAUGACGUUGCCCUGAUCA (SEQ ID NO: 1723; gRNA1540), UGCUCCCCAUGGCGUUGGAA (SEQ ID NO: 3467; gRNA3657), UUGCUCCCCAUGGCGUUGGA (SEQ ID NO: 3476; gRNA3658), CCCCAUGGCGUUGGAAGGCA (SEQ ID NO: 3443; gRNA3660), CCUCAGAUGUCUAUGUGUUU (SEQ ID NO: 1524; TSBTx3826), GCUUACAAUGACUGAGAUCU (SEQ ID NO: 1534; TSBTx3837), UGCUUACAAUGACUGAGAUCU (SEQ ID NO: 1535; TSBTx3837) and UCUCACCUCUGCAAGUAUUG (SEQ ID NO: 1529; TSBTx3835).

In any aspect or embodiment of the disclosure, the deaminase domain is an adenosine deaminase containing the TadA*7.10 amino acid sequence further containing a combination of amino acid alterations selected from one or more of: i. Y123H, Y147R, and Q154R, ii. I76Y, Y133H, Y147R, and Q154R, iii. V82S, and Q164R, iv. I76Y, V82S, Y123H, Y147R, and Q154R, v. I76Y, V82T, Y123H, Y147R, and Q154R, and vi. I76Y, V82T, Y123H, Y147T, and Q154S.

In any aspect or embodiment of the disclosure, the napDNAbp is a nickase. In any aspect or embodiment of the disclosure, the napDNAbp binds a protospacer adjacent motif (PAM) selected from one or more of NGA, NGC, NGG, and NNNRRT, where “N” is any nucleotide and “R” is A or G. In any aspect or embodiment of the disclosure, the napDNAbp is a Cas9 polypeptide.

In any aspect or embodiment of the disclosure, the one or more guide polynucleotides contain a modified nucleotide. In any aspect or embodiment of the disclosure, the one or more guide polynucleotides contain a sequence selected from one or more of:

End-mod SpCas9 guide polynucleotide
(SEQ ID NO: 440)
mNsmNsmNsNNNNNNNNNNNNNNNNNGUUUUAGAGCUAGAAAUAGCAAGU
UAAAAUAAGGCUAGUCCGUUAUCAACUUGAAAAAGUGGCACCGAGUCGGU
GCUsmUsmUsmU;
End-mod SaCas9 guide polynucleotide
(SEQ ID NO: 441)
mNsmNsmNsNNNNNNNNNNNNNNNNNNGUUUUAGUACUCUGUAAUGAAAA
UUACAGAAUCUACUAAAACAAGGCAAAAUGCCGUGUUUAUCUCGUCAACU
UGUUGGCGAGAUsmUsmUsmU;
HM01:
(SEQ ID NO: 440)
mNsmNsmNsNNNNNNNNNNNNNNNNNGUUUUAGAmGmCmUmAmGmAmAmA
mUmAmGmCAAGUUAAAAUAAGGCUAGUCCGUUAUCAmAmCmUmUmGmAmA
mAmAmAmGmUmGmGmCmAmCmCmGmAmGmUmCmGmGmUmGmCmUsmUsmU
smU;
HM07:
(SEQ ID NO: 440)
mNsmNsmNsmNmNmNmNmNmNmNNNNNNNNNNNmGUUUUAGmAmGmCmUm
AmGmAmAmAmUmAmGmCmAmAGUUmAAmAAmUAmAmGmGmCmUmAGUmCm
CGUUAmUmCAAmCmUmUmGmAmAmAmAmAmGmUmGGmCmAmCmCmGmAmG
mUmCmGmGmUmGmCmUsmUsmUsmU;
NLS (bpsv40):
(SEQ ID NOs: 440 and 446)
mNsmNsmNsNNNNNNNNNNNNNNNNNGUUUUAGAGCUAGAAAUAGCAAGU
UAAAAUAAGGCUAGUCCGUUAUCAACUUGAAAAAGUGGCACCGAGUCGGU
GCmUsmUsmUsmU-NHC6-CrossL-ac-CKRTADGSEFESPKKKRKV;
LONGEST:
(SEQ ID NO: 445)
mNsmNsmNsNNNNNNNNNNNNNNNNNGUUUUAGAmGmCmCmGmGmCmGmG
mAmAmAmCmGmCmCmGmGmCAAGUUAAAAUAAGGCUAGUCCGUUAUCAmA
mCmUmUmGmAmAmAmAmAmGUGmGmCmAmCmCmGmAmGmUmCmGmGmUmG
mCmUsmUsmUsmU;
NLS + LONGEST:
(SEQ ID NOs: 445 and 446)
mNsmNsmNsNNNNNNNNNNNNNNNNNGUUUUAGAmGmCmCmGmGmCmGmG
mAmAmAmCmGmCmCmGmGmCAAGUUAAAAUAAGGCUAGUCCGUUAUCAmA
mCmUmUmGmAmAmAmAmAmGmUmGmGmCmAmCmCmGmAmGmUmCmGmGmU
mGmCmUsmUsmUsmU-NHC5-CrossL-CKRTADGSEFESPKKKRKV;
and
LONGEST + GOLD:
(SEQ ID NO: 447)
mNsmNsmNsNNNNNNNNNNNNNNNNNGUUUUAGAmGmCmCmGmGmCmGmG
mAmAmAmCmGmCmCmGmGmCAAGUUAAAAUAAGGCUAGUCCGUUAmUmCA
AmCmUmUGGACUUCGGUCCmAmAmGUGGmCmAmCmCmGmAmGmUmCmGmG
mUmGmCmUsmUsmUsmU;

where “N” represents any nucleotide, “mN” indicates a 2′-OMe modification of the nucleotide “N”, and “Ns” indicates that the nucleotide “N” is linked to the following nucleotide by a phosphorothioate (PS), and where the number of N nucleotides is between 15 and 25.

In any aspect or embodiment of the disclosure, the nucleobase alteration effects an alteration to an encoded amino acid residue that results in disruption of Mg²⁺ binding to the CFB polypeptide encoded by the CFB polynucleotide. In any aspect or embodiment of the disclosure, the nucleobase alteration effects an alteration to an encoded amino acid residue that results in a reduction or elimination of serine protease activity of the CFB polypeptide encoded by the CFB polynucleotide. In any aspect or embodiment of the disclosure, the nucleobase alteration effects an alteration to an encoded amino acid residue that eliminates a salt bridge of the CFB polypeptide encoded by the CFB polynucleotide. In any aspect or embodiment of the disclosure, the nucleobase alteration effects an alteration to an encoded amino acid residue that reduces cleavage of the CFB polypeptide encoded by the CFB polynucleotide by a factor D polypeptide.

In any aspect or embodiment of the disclosure, the CFB polynucleotide is in a cell. In any aspect or embodiment of the disclosure, the cell is a mammalian cell. In any aspect or embodiment of the disclosure, the cell is a retinal cell or other cell of the eye, a nerve cell, or a hepatocyte.

In any aspect or embodiment of the disclosure, the one or more guide polynucleotides target the base editor to effect an alteration of the nucleobase of the CFB polynucleotide that disrupts a splice site in the CFB polynucleotide.

In any aspect or embodiment of the disclosure, the napDNAbp is a nickase.

In any aspect or embodiment of the disclosure, the napDNAbp binds a protospacer adjacent motif (PAM) selected from one or more of NGA, NGC, NGG, and NNNRRT, where “N” is any nucleotide and “R” is A or G.

In any aspect or embodiment of the disclosure, the napDNAbp is a Cas9 polypeptide.

In any aspect or embodiment of the disclosure, CFB activity, protein concentration, and/or mRNA concentration is reduced by at least about 15% as compared to a control subject without the alteration. In any aspect or embodiment of the disclosure, the inappropriate activation of the complement system is associated with increased levels of one or more of inflammation, the presence of autoantibodies, neural degeneration, and microthrombosis.

In any aspect or embodiment of the disclosure, the inappropriate activation of the complement system is associated with damage to the central nervous system (CNS), the eyes, the gastrointestinal system, the pulmonary system, the musculoskeletal system, the circulatory system, the integumentary system, blood cells, thyroid, kidney, joints, gastrointestinal system, or transplanted organs. In any aspect or embodiment of the disclosure, the disease or disorder is selected from one or more of acute antibody-mediated rejection, age-related macular degeneration, allergic bronchopulmonary aspergillosis, allergic neuritis, allergic rhinitis, Alzheimer's disease, amyotrophic lateral sclerosis (ALS), anaphylaxis, scleritis, atopic dermatitis, atypical hemolytic syndrome (aHUS), autoimmune hemolytic anemia, Bechet's disease, bronchiolitis, IC-MPGN/C3 glomerulopathy, central nervous system (CNS) inflammatory disorders, choroidal neovascularization (CNV), choroiditis, chronic allograft vasculopathy, chronic hepatitis, chronic muscle inflammation, chronic pain, chronic pancreatitis, chronic urticaria, Churg-Strauss syndrome, conjunctivitis, cyclitis, demyelinating disease, dermatitis, dermatomyositis, diabetic retinopathy, encephalitis, eosinophilic pneumonia, geographic atrophy, giant cell arteritis, glaucoma, glomerulonephritis, graft or transplant rejection or failure, HELLP syndrome, Henoch-Schonlein purpura, hypersensitivity pneumonitis, idiopathic pulmonary fibrosis (IPF), IgA nephropathy (IgAN), inflammatory bowel diseases, inflammatory joint conditions, inflammatory skin diseases, infusion reactions, interstitial pneumonia, iridocyclitis, iritis, ischemia/reperfusion injury, Kawasaki disease, keratitis, lupus nephritis, membranoproliferative glomerulonephritis (MPGN), meningitis, microscopic polyangiitis, myasthenia gravis, myocarditis, nasal polyposis, neuromyelitis optica, neuropathic pain, ocular inflammation, osteoarthritis, pancreatitis, panniculitis, paroxysmal nocturnal hemoglobinuria (PNH), pars planitis, pemphigoid, pemphigus, polyarteritis nodosa, polymyositis, primary membranous nephropathy, proliferative vitreoretinopathy, proteinuria, psoriasis, pulmonary fibrosis, renal disease, respiratory distress syndrome, retinal neovascularization (RNV), retinopathy of prematurity, rheumatoid arthritis (RA), rhinosinusitis, sarcoid, sarcoidosis, scleritis, scleroderma, sclerodermatomyositis, sclerosis, Sjögren syndrome, systemic lupus erythematosus, systemic scleroderma, Takayasu's arteritis, Tautopathies, thyroiditis, thyroidoisis, ulcerative colitis, uveitis, vasculitis, and Wegener's granulomatosis.

In any aspect or embodiment of the disclosure, the administration is local administration to an eye, to spinal fluid, or to the liver.

In any aspect or embodiment of the disclosure, the CFB polynucleotide is contacted with two or more guide polynucleotides, and where each guide polynucleotide binds a different location within the CFB polynucleotide.

In any aspect or embodiment of the disclosure, the subject is a mammal.

In any aspect or embodiment of the disclosure, the deaminase domain contains a cytidine deaminase or a TadA variant (TadA*) containing an amino acid sequence having at least 90% sequence identity to the following TadA*7.10 amino acid sequence: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), where the TadA* further contains a combination of amino acid alterations compared to the TadA*7.10 amino acid sequence selected from one or more of: i. Y123H, Y147R, and Q154R, ii. 176Y, Y133H, Y147R, and Q154R, iii. V82S, and Q164R, iv. I76Y, V82S, Y123H, Y147R, and Q154R, v. I76Y, V82T, Y123H, Y147R, and Q154R, and vi. I76Y, V82T, Y123H, Y147T, and Q154S.

In any aspect provided herein, or embodiments thereof, the method is not a process for modifying the germline genetic identity of human beings.

In any aspect provided herein, or embodiments thereof, the adenosine deaminase domain contains a combination of mutations selected from those listed in Table 5G.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “adenine” or “9H-Purin-6-amine” is meant a purine nucleobase with the molecular formula C₅H₅N₅, having the structure

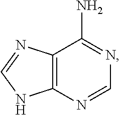

and corresponding to CAS No. 73-24-5.

By “adenosine” or “4-Amino-1-[(2R,3R,4S,5R)-3,4-dihydroxy-5-(hydroxymethyl)oxolan-2-yl]pyrimidin-2 (1H)-one” is meant an adenine molecule attached to a ribose sugar via a glycosidic bond, having the structure

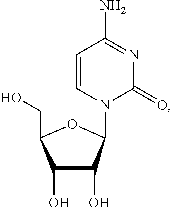

and corresponding to CAS No. 65-46-3. Its molecular formula is C₁₀H₁₃N₅O₄.

By “adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In some embodiments, the adenosine deaminase is an adenosine deaminase variant with one or more alterations and is capable of deaminating both adenine and cytosine in a target polynucleotide (e.g., DNA, RNA) and may be referred to as a “dual deaminase”. Non-limiting examples of dual deaminases include those described in PCT/US22/22050. In some embodiments, the target polynucleotide is single or double stranded. In some embodiments, the adenosine deaminase variant is capable of deaminating both adenine and cytosine in DNA. In some embodiments, the adenosine deaminase variant is capable of deaminating both adenine and cytosine in single-stranded DNA. In some embodiments, the adenosine deaminase variant is capable of deaminating both adenine and cytosine in RNA. In embodiments, the adenosine deaminase variant is selected from those described in PCT/US2020/018192, PCT/US2020/049975, PCT/US2017/045381, PCT/US2021/016827, PCT/US2022/073781, PCT/US24/34189, or PCT/US2020/028568, the full contents of which are each incorporated herein by reference in their entireties for all purposes. Further non-limiting examples of adenosine deaminases include those disclosed or referenced in Rufflow, et al., “Design of highly functional genome editors by modeling of the universe of CRISPR-Cas Sequences,” bioRxiv, posted Apr. 22, 2024, doi: 10.1101/2024.04.22.590591, the disclosure of which is incorporated herein by reference in its entirety for all purposes, which were designed using artificial intelligence. Further exemplary adenosine deaminase amino acid sequences include: TadA-8e (SEQ ID NO: 3575), Tad1 (SEQ ID NO: 3576), Tad2 (SEQ ID NO: 3577), Tad3 (SEQ ID NO: 3578), Tad4 (SEQ ID NO: 3579), Tad6 (SEQ ID NO: 3580), Tad6-SR (SEQ ID NO: 3581), TadA9 (SEQ ID NO: 3582), TadA20 (SEQ ID NO: 3583), Staphylococcus aureus TadA (SEQ ID NO: 3584), Bacillus subtilis TadA (SEQ ID NO: 3585), Salmonella typhimurium TadA (SEQ ID NO: 3586), Shewanella putrefaciens (SEQ ID NO: 3587), Haemophilus influenzae F3031 TadA (SEQ ID NO: 3588), Caulobacter crescentus TadA (SEQ ID NO: 3589), Geobacter sulfurreducens TadA (SEQ ID NO: 3590), Streptococcus pyogenes TadA (SEQ ID NO: 3591), Aquifex aeolicus TadA (SEQ ID NO: 3592), and E. coli TadA deaminase (ecTadA) (SEQ ID NO: 3593).

By “adenosine deaminase activity” is meant catalyzing the deamination of adenine or adenosine to guanine in a polynucleotide.

By “Adenosine Base Editor (ABE)” is meant a base editor comprising an adenosine deaminase.

By “Adenosine Base Editor (ABE) polynucleotide” is meant a polynucleotide encoding an ABE.

By “Adenosine Base Editor 8 (ABE8) polypeptide” or “ABE8” is meant a base editor as defined herein comprising an adenosine deaminase or adenosine deaminase variant comprising one or more of the alterations listed in Table 5B, one of the combinations of alterations listed in Table 5B, or an alteration at one or more of the amino acid positions listed in Table 5B, where such alterations are relative to the following reference sequence: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), or a corresponding position in another adenosine deaminase. In embodiments, ABE8 comprises alterations at amino acids 82 and/or 166 of SEQ ID NO: 1. In some embodiments, ABE8 comprises further alterations, as described herein, relative to the reference sequence.

By “Adenosine Base Editor 8 (ABE8) polynucleotide” is meant a polynucleotide encoding an ABE8 polypeptide.

“Administering” is referred to herein as providing one or more compositions described herein to a patient or a subject. By way of example and without limitation, composition administration (e.g., injection) can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally. Alternatively, or concurrently, administration can be by the oral route.

By “agent” is meant any small molecule chemical compound, antibody, nucleic acid molecule, polypeptide, or functional fragments thereof.

By “alteration” is meant a change in the level, structure, or activity of an analyte, gene or polypeptide as detected by standard art known methods such as those described herein. As used herein, an alteration includes a change (e.g., increase or reduction) in expression levels. In embodiments, the increase or reduction in expression levels is by 10%, 25%, 40%, 50% or greater. In some embodiments, an alteration includes an insertion, deletion, or substitution of a nucleobase or amino acid (by, e.g., genetic engineering).

By “ameliorate” is meant reduce, suppress, attenuate, diminish, arrest, or stabilize the development or progression of a disease.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide.

Such biochemical modifications could increase the analog's protease resistance, membrane permeability, or half-life, without altering, for example, ligand binding. An analog may include an unnatural amino acid.

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1). Representative nucleic acid and protein sequences of base editors include those sequences having about or at least about 85% sequence identity to any base editor sequence provided in the sequence listing, such as those corresponding to SEQ ID NOs: 2-11.

By “BE4 cytidine deaminase (BE4) polypeptide,” is meant a base editor comprising a nucleic acid programmable DNA binding protein (napDNAbp) domain, a cytidine deaminase domain, and two uracil glycosylase inhibitor domains (UGIs). In embodiments, the napDNAbp is a Cas9n (D10A) polypeptide. Non-limiting examples of cytidine deaminase domains include rAPOBEC, ppAPOBEC, RrA3F, AmAPOBEC1, and SsAPOBEC3B.

By “BE4 cytidine deaminase (BE4) polynucleotide,” is meant a polynucleotide encoding a BE4 polypeptide.

By “base editing activity” is meant acting to chemically alter a base within a polynucleotide. In one embodiment, a first base is converted to a second base. In one embodiment, the base editing activity is cytidine deaminase activity, e.g., converting target C·G to T·A. In another embodiment, the base editing activity is adenosine or adenine deaminase activity, e.g., converting A·T to G·C.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine or cytosine base editor (CBE). In some embodiments, the base editor system (e.g., a base editor system comprising a cytidine deaminase) comprises a uracil glycosylase inhibitor or other agent or peptide (e.g., a uracil stabilizing protein such as provided in WO2022015969, the disclosure of which is incorporated herein by reference in its entirety for all purposes) that inhibits the inosine base excision repair system.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casnl nuclease or a CRISPR (clustered regularly interspaced short palindromic repeat) associated nuclease.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free-OH can be maintained; and glutamine for asparagine such that a free-NH2 can be maintained.

Amino acids generally can be grouped into classes according to the following common side-chain properties:
(1) hydrophobic: Norleucine, Met, Ala, Val, Leu, He;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following: TAG, TAA, and TGA.

By “complement factor B (CFB) polypeptide” or “factor B (FB) polypeptide” is meant a factor B protein with at least about 85% amino acid sequence identity to GenBank Accession No. AAA16820.1, which is provided below, or a fragment thereof that is capable of mediating activation of the complement system. In embodiments, CFB is capable of cleaving an Arg-Ser bond in complement component C3 to yield C3a and C3b and/or an Arg-Ser bond in complement component C5 to yield C5a and C5b.

>AAA16820.1 complement factor B [Homo sapiens]
(SEQ ID NO: 426)
MGSNLSPQLCLMPFILGLLSGGVTTTPWSLAQPQGSCSLEGVEIKGGSFR
LLQEGQALEYVCPSGFYPYPVQTRTCRSTGSWSTLKTQDQKTVRKAECRA
IHCPRPHDFENGEYWPRSPYYNVSDEISFHCYDGYTLRGSANRTCQVNGR
WSGQTAICDNGAGYCSNPGIPIGTRKVGSQYRLEDSVTYHCSRGLTLRGS
QRRTCQEGGSWSGTEPSCQDSFMYDTPQEVAEAFLSSLTETIEGVDAEDG
HGPGEQQKRKIVLDPSGSMNIYLVLDGSDSIGASNFTGAKKCLVNLIEKV
ASYGVKPRYGLVTYATYPKIWVKVSEADSSNADWVTKQLNEINYEDHKLK
SGTNTKKALQAVYSMMSWPDDVPPEGWNRTRHVIILMTDGLHNMGGDPIT
VIDEIRDLLYIGKDRKNPREDYLDVYVFGVGPLVNQVNINALASKKDNEQ
HVFKVKDMENLEDVFYQMIDESQSLSLCGMVWEHRKGTDYHKQPWQAKIS
VIRPSKGHESCMGAVVSEYFVLTAAHCFTVDDKEHSIKVSVGGEKRDLEI
EVVLFHPNYNINGKKEAGIPEFYDYDVALIKLKNKLKYGQTIRPICLPCT
EGTTRALRLPPTTTCQQQKEELLPAQDIKALFVSEEEKKLTRKEVYIKNG
DKKGSCERDAQYAPGYDKVKDISEVVTPRFLCTGGVSPYADPNTCRGDSG
GPLIVHKRSRFIQVGVISWGVVDVCKNQKRQKQVPAHARDFHINLFQVLP
WLKEKLQDEDLGFL

By “complement factor B (CFB) polynucleotide” or “factor B (FB) polynucleotide” is meant a nucleic acid molecule encoding a CFB polypeptide, as well as the introns, exons, 3′ untranslated regions, 5′ untranslated regions, and regulatory sequences associated with its expression, or fragments thereof. In embodiments, a CFB polynucleotide is the genomic sequence, cDNA, mRNA, or gene associated with and/or required for CFB expression. An exemplary CFB nucleotide sequences from Homo Sapiens is provided below (GenBank: L15702.1:41-2335; Ensembl: ENST00000425368.7):

>L15702.1:41-2335 Human complement factor B mRNA,
complete cds
(SEQ ID NO: 427)
ATGGGGAGCAATCTCAGCCCCCAACTCTGCCTGATGCCCTTTATCTTGGGCCTCTTGTCTGG
AGGTGTGACCACCACTCCATGGTCTTTGGCCCAGCCCCAGGGATCCTGCTCTCTGGAGGGGG
TAGAGATCAAAGGCGGCTCCTTCCGACTTCTCCAAGAGGGCCAGGCACTGGAGTACGTGTGT
CCTTCTGGCTTCTACCCGTACCCTGTGCAGACACGTACCTGCAGATCTACGGGGTCCTGGAG
CACCCTGAAGACTCAAGACCAAAAGACTGTCAGGAAGGCAGAGTGCAGAGCAATCCACTGTC
CAAGACCACACGACTTCGAGAACGGGGAATACTGGCCCCGGTCTCCCTACTACAATGTGAGT
GATGAGATCTCTTTCCACTGCTATGACGGTTACACTCTCCGGGGCTCTGCCAATCGCACCTG
CCAAGTGAATGGCCGGTGGAGTGGGCAGACAGCGATCTGTGACAACGGAGCGGGGTACTGCT
CCAACCCGGGCATCCCCATTGGCACAAGGAAGGTGGGCAGCCAGTACCGCCTTGAAGACAGC
GTCACCTACCACTGCAGCCGGGGGCTTACCCTGCGTGGCTCCCAGCGGCGAACGTGTCAGGA
AGGTGGCTCTTGGAGCGGGACGGAGCCTTCCTGCCAAGACTCCTTCATGTACGACACCCCTC
AAGAGGTGGCCGAAGCTTTCCTGTCTTCCCTGACAGAGACCATAGAAGGAGTCGATGCTGAG
GATGGGCACGGCCCAGGGGAACAACAGAAGCGGAAGATCGTCCTGGACCCTTCAGGCTCCAT
GAACATCTACCTGGTGCTAGATGGATCAGACAGCATTGGGGCCAGCAACTTCACAGGAGCCA
AAAAGTGTCTAGTCAACTTAATTGAGAAGGTGGCAAGTTATGGTGTGAAGCCAAGATATGGT
CTAGTGACATATGCCACATACCCCAAAATTTGGGTCAAAGTGTCTGAAGCAGACAGCAGTAA
TGCAGACTGGGTCACGAAGCAGCTCAATGAAATCAATTATGAAGACCACAAGTTGAAGTCAG
GGACTAACACCAAGAAGGCCCTCCAGGCAGTGTACAGCATGATGAGCTGGCCAGATGACGTC
CCTCCTGAAGGCTGGAACCGCACCCGCCATGTCATCATCCTCATGACTGATGGATTGCACAA
CATGGGCGGGGACCCAATTACTGTCATTGATGAGATCCGGGACTTGCTATACATTGGCAAGG
ATCGCAAAAACCCAAGGGAGGATTATCTGGATGTCTATGTGTTTGGGGTCGGGCCTTTGGTG
AACCAAGTGAACATCAATGCTTTGGCTTCCAAGAAAGACAATGAGCAACATGTGTTCAAAGT
CAAGGATATGGAAAACCTGGAAGATGTTTTCTACCAAATGATCGATGAAAGCCAGTCTCTGA
GTCTCTGTGGCATGGTTTGGGAACACAGGAAGGGTACCGATTACCACAAGCAACCATGGCAG
GCCAAGATCTCAGTCATTCGCCCTTCAAAGGGACACGAGAGCTGTATGGGGGCTGTGGTGTC
TGAGTACTTTGTGCTGACAGCAGCACATTGTTTCACTGTGGATGACAAGGAACACTCAATCA
AGGTCAGCGTAGGAGGGGAGAAGCGGGACCTGGAGATAGAAGTAGTCCTATTTCACCCCAAC
TACAACATTAATGGGAAAAAAGAAGCAGGAATTCCTGAATTTTATGACTATGACGTTGCCCT
GATCAAGCTCAAGAATAAGCTGAAATATGGCCAGACTATCAGGCCCATTTGTCTCCCCTGCA
CCGAGGGAACAACTCGAGCTTTGAGGCTTCCTCCAACTACCACTTGCCAGCAACAAAAGGAA
GAGCTGCTCCCTGCACAGGATATCAAAGCTCTGTTTGTGTCTGAGGAGGAGAAAAAGCTGAC
TCGGAAGGAGGTCTACATCAAGAATGGGGATAAGAAAGGCAGCTGTGAGAGAGATGCTCAAT
ATGCCCCAGGCTATGACAAAGTCAAGGACATCTCAGAGGTGGTCACCCCTCGGTTCCTTTGT
ACTGGAGGAGTGAGTCCCTATGCTGACCCCAATACTTGCAGAGGTGATTCTGGCGGCCCCTT
GATAGTTCACAAGAGAAGTCGTTTCATTCAAGTTGGTGTAATCAGCTGGGGAGTAGTGGATG
TCTGCAAAAACCAGAAGCGGCAAAAGCAGGTACCTGCTCACGCCCGAGACTTTCACATCAAC
CTCTTTCAAGTGCTGCCCTGGCTGAAGGAGAAACTCCAAGATGAGGATTTGGGTTTTCTATA
A
>chromosome:GRCh38:6:31945050:31952686:1
(ENST00000425368.7), where exons are
shown in bold text, untranslated regions
are underlined, introns correspond to plain
text regions between bold text regions,
and a TATA box is shown by double-underlined
text. The sequence contains 18 exons, where
Exon 1 corresponds to the first exon from
the 5′ end of the sequence, Exon 2 corresponds
to the second exon from the 5′ end of the
sequence, and so on to Exon 18.
(SEQ ID NO: 428)
AGGACCCAGGGGTTACAGGATCTCAGCCTTGTTGGGGGGATGAGGGAGGCCTTTGAGGGATC
TAGGGAGGTTGGGGCTTACAGTTGGGGCTGTGGCAGCCTCCCAGCCAGTTCTCTCCTTTTCT
CCAGGTGGGTCTGGTGAGCTGGGGTCTTTACAACCCCTGCCTTGGCTCTGCTGACAAAAACT
CCCGCAAAAGGGCCCCTCGTAGCAAGGTCCCGCCGCCACGAGACTTTCACATCAATCTCTTC
CGCATGCAGCCCTGGCTGAGGCAGCACCTGGGGGATGTCCTGAATTTTTTACCCCTCTAGCC
ATGGCCACTGAGCCCTCTGCTGCCCTGCCAGAATCTGCCGCCCCTCCATCTTCTACCTCTGA
ATGGCCACCCTTAGACCCTGTGATCCATCCTCTCTCCTAGCTGAGTAAATCCGGGTCTCTAG
GATGCCAGAGGCAGCGCACACAAGCTGGGAAATCCTCAGGGCTCCTACCAGCAGGACTGCCT
CGCTGCCCCACCTCCCGCTCCTTGGCCTGTCCCCAGATTCCTTCCCTGGTTGACTTGACTCA
TGCTTGTTTCACTTTCACATGGAATTTCCCAGTTATGAAATTAATAAAAATCAATGGTTTCC
ACATCTCTCAGTGCCTCTATCTGGAGGCCAGGTAGGGCTGGCCTTGGGGGAGGGGGAGGCCA
GAATGACTCCAAGAGCTACAGGAAGGCAGGTCAGAGACCCCACTGGACAAACAGTGGCTGGA
CTCTGCACCATAACACACAATCAACAGGGGAGTGAGCTGGATCCTTATTTCTGGTCCCTAAG
TGGGTGGTTTGGGCTTACTGGGGAGGAGCTAAGGCCGGAGAGGAGGTACTGAAGGGGAGAGT
CCTGGACCTTTGGCAGCAAAGGGTGGGACTTCTGCAGTTTCTGTTTCCTTGACTGGCAGCTC
AGCGGGGCCCTCCCGCTTGGATGTTCCGGGAAAGTGATGTGGGTAGGACAGGCGGGGCGAGC
CGCAGGTGCCAGAACACAGATTGTATAAAAGGCTGGGGGCTGGTGGGGAGCAGGGGAAGGGA
ATGTGACCAGGTCTAGGTCTGGAGTTTCAGCTTGGACACTGAGCCAAGCAGACAAGCAAAGC
AAGCCAGGACACACCATCCTGCCCCAGGCCCAGCTTCTCTCCTGCCTTCCAACGCCATGGGG
AGCAATCTCAGCCCCCAACTCTGCCTGATGCCCTTTATCTTGGGCCTCTTGTCTGGAGGTAA
GCGAGGGTAACCTTCCCTTCCTGCTGTCTCCAGCATCCCTCCTTGGCCTTTTGGGGCCAGGC
TTCATCAGCCTTTCTCTTCAGGTGTGACCACCACTCCATGGTCTTTGGCCCGGCCCCAGGGA
TCCTGCTCTCTGGAGGGGGTAGAGATCAAAGGCGGCTCCTTCCGACTTCTCCAAGAGGGCCA
GGCACTGGAGTACGTGTGTCCTTCTGGCTTCTACCCGTACCCTGTGCAGACACGTACCTGCA
GATCTACGGGGTCCTGGAGCACCCTGAAGACTCAAGACCAAAAGACTGTCAGGAAGGCAGAG
TGCAGAGGTTTGAGGGCAATGAGTGTGGGCAGTGGCCTAAGGCAGAAACAGGGCAGGCGGCA
GCAAGGTCAGGACTAGGATGAGACTAGGCAGGGTGACAAGGTGGGCTGACCGGGAGTAGGAG
CAGTTTTAGGGTGGCAGGCGGAAAGGGGGCAAGAAAAAGCGGAGTTAACCCTTACTAAGCAT
TTACCCTGGGCTTCCAGGCAGCCCTGGAAGTCAAGAGAACACTCAGAAATGGGGAGGGAGAA
GCAGTGGAAATCCATATGGGTTGAGGAGTAGGTAAGATGCTGCTTCTGCGGGACTGGGAATG
CGCTGTTTCTCAGTGACATGGTCTCCGAGACCAGGAGGGATACACCTAAGGCAGCCTTTCCC
TCTTGATGACTTCTACTTGTCCCCCCTTCTCAAAGCAATCCACTGTCCAAGACCACACGACT
TCGAGAACGGGGAATACTGGCCCCGGTCTCCCTACTACAATGTGAGTGATGAGATCTCTTTC
CACTGCTATGACGGTTACACTCTCCGGGGCTCTGCCAATCGCACCTGCCAAGTGAATGGCCG
ATGGAGTGGGCAGACAGCGATCTGTGACAACGGAGGTGAGAAGCATCCCCTCCCCCTACATT
GCTGTCTCCCTGACGGCGCCCAGCCCGAGGAGTGGGCACTCGGCTCCGGACACTGTAACTCT
TGCTCTCTACCTTGCTCACGGGGCCTCAGGCTTCAGTGCTTACCTCGATGTCTCATACCTCT
GCAGCGGGGTACTGCTCCAACCCGGGCATCCCCATTGGCACAAGGAAGGTGGGCAGCCAGTA
CCGCCTTGAAGACAGCGTCACCTACCACTGCAGCCGGGGGCTTACCCTGCGTGGCTCCCAGC
GGCGAACGTGTCAGGAAGGTGGCTCTTGGAGCGGGACGGAGCCTTCCTGCCAAGGTGACCTT
TGACCTGTACCCCCAGGTCAGATCCTGGTCTTCCATCCTACTGTCTTCTCTCCCCACCTCAA
CCCTGCTCTTTCCTCACTTTGTTTAAACCTCCCTGTACAACTATCTCACTTCTGAGCCTTTT
ATACCCTGGAAACCCATGATCCCCCGTCTCTTTGGTCACTGTATCCCTGACACTCCCAGACA
TTTGACCTCATTTCTGACTCTCCCAGACTCCTTCATGTACGACACCCCTCAAGAGGTGGCCG
AAGCTTTCCTGTCTTCCCTGACAGAGACCATAGAAGGAGTCGATGCTGAGGATGGGCACGGC
CCAGGTTTGAAGACAGAGAAGGGAGGCAGGGCAGGGAACTGGGGGAAAATGGAGAAGGGACA
GAACTGTTAATGCTGGAGCCTGAGCCACTCTCCTGGCACCCAGGGGAACAACAGAAGCGGAA
GATCGTCCTGGACCCTTCAGGCTCCATGAACATCTACCTGGTGCTAGATGGATCAGACAGCA
TTGGGGCCAGCAACTTCACAGGAGCCAAAAAGTGTCTAGTCAACTTAATTGAGAAGGTGGAA
TCCTCCTATCCCTGAACTCGGGGGAATGGAATCTCGCTGATCTTCCAGGACTAGCTCCCTGA
TCATTCCAGCCCCTCTGAACAACAGGGCCCCAGGAAAATCTCCAGGTCCTATTCTGTCCTCC
TTCCCTTTTACTTGAAGCAGTTTCTTGACTGGTAATTCCTCCATGAACCTCAGCCCTTGAGC
CTCTTACTGAGAGCCTCCCTGTCCCAGCAAAGTCGCTGAAATCTCCCAATCACAGTATTCTA
TTTTCAATGCCATGGCGCCTTGTTCTCCTCACCCACAGGTGGCAAGTTATGGTGTGAAGCCA
AGATATGGTCTAGTGACATATGCCACATACCCCAAAATTTGGGTCAAAGTGTCTGAAGCAGA
CAGCAGTAATGCAGACTGGGTCACGAAGCAGCTCAATGAAATCAATTATGAAGGTCAGAGGT
TAGGGAATGGTGGGAGGTTCACTTTGGGGTCAGGAGGTTCAGGGTGGAGGGGGTCATGAGAC
TACCTTGAGGGCGACAGGGAGGACCACTTTGTAGTCAAAAGTTGAACAGCAGGATCGTTGGG
CAATGGAGGTTAGTGGGAACCTGTTGGGGGCTGGAAGGGCCACTTTGTGGTCAAAGGGAAGT
CCGTGTAATGATGATTAACTTAAAAAGTTGAAAGATGTGGGATTTCAGTTGCAGATTGGTCT
CTGGGGTTAAAAGATGGCTTGGAAGACCAGGTGAGGTGATGGTCTCTTCCCTCTCCACAGAC
CACAAGTTGAAGTCAGGGACTAACACCAAGAAGGCCCTCCAGGCAGTGTACAGCATGATGAG
CTGGCCAGATGACGTCCCTCCTGAAGGCTGGAACCGCACCCGCCATGTCATCATCCTCATGA
CTGATGGTCAGAAGGGACCTCTCTCCTGTCCCAGCCTCCCCACCTTCTCAGACCAGCATGTG
GCCCTTAAGTCCACTTGTAACACTATACCCATGGTTGGGGCCCTGAATGTGACTCATAGCTG
GCTGTTCATCTCTCCTGTGACCCTTCATAAGGAATTCTTCCTAAGCCCTGTGATCAACTATC
TCTAACCCTTCCTCAACTTGCTCACCCTGCCATGTGTATCCCTGCCTTTAGCCAGTTTATCT
TCCTTATCTCCTACCCTCATGGTCCTGTCTCTTCTGCAGGATTGCACAACATGGGGGGGGAC
CCAATTACTGTCATTGATGAGATCCGGGACTTGCTATACATTGGCAAGGATCGCAAAAACCC
AAGGGAGGATTATCTGGGTGAGTAACCTGCCTAGGACCCAGCACCCCACTTCCTCAGGGCTT
GGACCCTCATCCTTCCTTTTTATCCCTCAGATGTCTATGTGTTTGGGGTCGGGCCTTTGGTG
AACCAAGTGAACATCAATGCTTTGGCTTCCAAGAAAGACAATGAGCAACATGTGTTCAAAGT
CAAGGATATGGAAAACCTGGAAGATGTTTTCTACCAAATGATCGGTAGGGAGATACAAGGGA
ATAAAGAACACAACTCTCCTCAGGTTCCCCTGAAGTAATTCATTCTTCCTCTACACCTGAAG
CTCTAGTTGCCTGGAAAGCCTTCTTCATTCCTCCTTCTCTACCTCAGTGTCACTATTCTTGT
TTCCTGGCACTGTTCACTTAACCTTAGAATCACAGAGCTCTGAGCACTTCAGAGATCTTTCT
ATAGTCCTACATTTGACACGTGGAAACAGAAGCCAAAGGAGGTCAAGGGACAGCAAGTTAGC
AACAAGGGTGGGCTTGAAAACAGCCAGGCCTCTGACAGCTTGATCCCAAGTTCTTTCCCTTT
TCAGTCCACCATAGCAGTTTTCTCCTAACACGAGGAAACAAATACCCGTGGTCTTTCCCTTT
CTCCTTTTGGGCCTTTGCTCCCCATAGACTCCTACCCAAAAGGCTGCTGCCATTTGGGAATG
AAGTGTTCCGAGTTTTCAGCACATTCTCCTTCTCTGCCAGATGAAAGCCAGTCTCTGAGTCT
CTGTGGCATGGTTTGGGAACACAGGAAGGGTACCGATTACCACAAGCAACCATGGCAGGCCA
AGATCTCAGTCATTGTAAGCACAGAATCCCAGTAGTGGGGACTTGGGGGAGGTGAGGTCAAG
GTGAAATGGGAGTAGGGGAAGGAAAAAATGGCCATAAGAGATGGTGGTTTGTGAAAGTTGAG
CTTTCCCTCTCTACTGTTGTGTCCCCAGCGCCCTTCAAAGGGACACGAGAGCTGTATGGGGG
CTGTGGTGTCTGAGTACTTTGTGCTGACAGCAGCACATTGTTTCACTGTGGATGACAAGGAA
CACTCAATCAAGGTCAGCGTAGGTAAGGATGCAACTGAAGGTCCTGGGCTGCACCTATGCTC
TCCAGGCAACACCTCCCACTTTCTACAGATCCTACACTCCACCCATCCTCAATGCAGCCCCA
TTCCTTGCACCCCAGACCAGTCAGGGATGGGGGAAGACGTGAAGTTAGGAATGACACGGGGC
CAGAGGCAGGAAGCTGCCCACAAAGAGGTGGTACCTACTCTCCTACTTCAGGAGGGGAGAAG
CGGGACCTGGAGATAGAAGTAGTCCTATTTCACCCCAACTACAACATTAATGGGAAAAAAGA
AGCAGGAATTCCTGAATTTTATGACTATGACGTTGCCCTGATCAAGCTCAAGAATAAGCTGA
AATATGGCCAGACTATCAGGTGAGAGCGTCCAGATCCCTGAGGAAAGGCTGGGAAAGGCTGG
AGGACTGGGGTGAGGAGCAGGCCTGGTTTGCTGTTCTCCTTGTCCTTTATAGGCCCATTTGT
CTCCCCTGCACCGAGGGAACAACTCGAGCTTTGAGGCTTCCTCCAACTACCACTTGCCAGCA
ACAAAGTAAGACATACTTGGCAAGAGGATAAGGATGAGATCCCAAGAGACAAGTGGGGCATG
AGAGGGAGGTGCAATAGGAAGAGATGATGCCTGGCCCAGAACCTAGCTCTAGAAGGGCTTAG
GGGACATCTACTGAGTGACAAAGGCAATGGGGAGATGACAGTGGTGGGAGCAGCTGAAGTGA
CGCAGTCTATTCGTCCAGAGGAAGAGCTGCTCCCTGCACAGGATATCAAAGCTCTGTTTGTG
TCTGAGGAGGAGAAAAAGCTGACTCGGAAGGAGGTCTACATCAAGAATGGGGATAAGGTGAG
AAACGGGCATCCTAAGGAGGCACTCTAGGCCCCAATCCTTCCTAAGCCACTTCTGTTCATTA
CTTCTCCATGCTTCCCACCTCCCCTACAGAAAGGCAGCTGTGAGAGAGATGCTCAATATGCC
CCAGGCTATGACAAAGTCAAGGACATCTCAGAGGTGGTCACCCCTCGGTTCCTTTGTACTGG
AGGAGTGAGTCCCTATGCTGACCCCAATACTTGCAGAGGTGAGAGAATGCTCTTTGGTTGTG
CTACAAGTGCCCAAGGCCCAACAGTCCTTTTCTCTACAGCTTCTCCTCTCCTTGCAGGTGAT
TCTGGCGGCCCCTTGATAGTTCACAAGAGAAGTCGTTTCATTCAAGTGAGTCCTCCCTTTCC
TATCTGGGGAGATGCCAAGTGGTCAGCATGGGCCCCAAAGCAGGAAAGCTCAATGCATGTGG
CTAGTAATTCGAGGTAGGCAGAGCCTGCCTCACCTTAGGACCGCATGTCTTGCCTGCGTGTG
TCAAGAACGAGGCTGAGCTGGGTCCCTAGTCTGATTCCTTTAGGTCAGCTAAGACACAAGCA
GGAACAGCCATGCTTCCAGGATTAGGAATTCTACTGAATGATCCATGGCACCCCACTGCCTC
TGCAGGTTGGTGTAATCAGCTGGGGAGTAGTGGATGTCTGCAAAAACCAGAAGCGGCAAAAG
CAGGTACCTGCTCACGCCCGAGACTTTCACATCAACCTCTTTCAAGTGCTGCCCTGGCTGAA
GGAGAAACTCCAAGATGAGGATTTGGGTTTTCTATAAGGGGTTTCCTGCTGGACAGGGGCGT
GGGATTGAATTAAAACAGCTGCGACAACACCTGTGTTCCAGATCCTTTTGGGGCAAGGGAGT
GGGGAACAGGCACTGGCCATGTTGTTACACTGAGATCAAACCTGACAGCCGTTTTTAAAGGT
TTAACCCCAATCCCAAGTGCTGAAAAACCAGAGGCTGAGGGAGATGTGTAAGCTTCCACCTC
AGTGTTTTACTGAGACCAGCATTGGGGCATATGAGGCACAAGGAATCCAGCTCTGTTCCCTA
GAAGCCATCCACAAGGTTTTCCTTGTAGACGTCATCACTGTAGACAATCTGGGTCCTCTTGT
CCCGGTGGCAACCCTTAGGGCTGTTCTGGACAGCTAGGGAGGGAGGAGAGGAACAGTTAAGG
TCTAAAGGAGATCATAGAACAGACCCTGAGGCTGACTCCTGACCACCTCACTCCTGGCCACT
GGCCCCTGGAAGCCCAGTTTCCACGCTGCCCTCTGGTGGCCAGGATGGCCTGTCTTCCTTAG
CTCCTTTGTGCCAACCCATGGCCAAGAAAAGTATAAGTGGACATTTTGATGAATGTTTTGTT
CTTAGAAAAATCCCAAATGTCATTGTTGAGACACGTGAATGATATTAACCCACTACTTACAG
TCAGTATGTCA

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and x-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non-covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

By “cytosine” or “4-Aminopyrimidin-2 (1H)-one” is meant a purine nucleobase with the molecular formula C₄H₅N₃O, having the structure

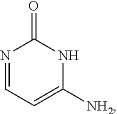

and corresponding to CAS No. 71-30-7.

By “cytidine” is meant a cytosine molecule attached to a ribose sugar via a glycosidic bond, having the structure

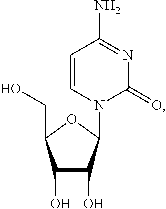

and corresponding to CAS No. 65-46-3. Its molecular formula is C₉H₁₃N₃O₅.

By “Cytidine Base Editor (CBE)” is meant a base editor comprising a cytidine deaminase. Non-limiting examples of cytidine deaminase base editor amino acid sequences include amino acid sequences for BE4max (SEQ ID NO: 3658), YE1-BE4 (SEQ ID NO: 3659), YE2-BE4 (SEQ ID NO: 3660), YEE-BE4 (SEQ ID NO: 3661), EE-BE4 (SEQ ID NO: 3662), R33A-BE4 (SEQ ID NO: 3663), R33A+K34A-BE4 (SEQ ID NO: 3664), APOBEC3A (A3A)-BE4 (SEQ ID NO: 3665), APOBEC3B (A3B)-BE4 (SEQ ID NO: 3666), APOBEC3G (A3G)-BE4 (SEQ ID NO: 3667), AID-BE4 (SEQ ID NO: 3668), CDA-BE4 (SEQ ID NO: 3669), FERNY-BE4 (SEQ ID NO: 3670), evolved APOBEC3A (eA3A)-BE4 (SEQ ID NO: 3671), AALN-BE4 (SEQ ID NO: 3672), BE4max modified with SpCas9-NG (SEQ ID NO: 3673), YE1-SpCas9-NG (YE1-NG) (SEQ ID NO: 3674), YE2-SpCas9-NG (SEQ ID NO: 3675), YEE-SpCas9-NG (SEQ ID NO: 3676), EE-SpCas9-NG (SEQ ID NO: 3677), R33A+K34A-SpCas9-NG (SEQ ID NO: 3678), YE1-CP1028 (YE1-BE4-CP1028, or YE1-CP) (SEQ ID NO: 3679), YE2-CP1028 (YE2-BE4-CP1028) (SEQ ID NO: 3680), YEE-CP1028 (YEE-BE4-CP1028) (SEQ ID NO: 3681), EE-CP1028 (EE-BE4-CP1028) (SEQ ID NO: 3682), R33A+K34A-CP1028 (R33A+K34A-BE4-CP1028) (SEQ ID NO: 3683), BE4max (with nickase) (SEQ ID NO: 3702), BE4 (SEQ ID NO: 3703), BE4 with His tag (SEQ ID NO: 3704), BE4max (SEQ ID NO: 3705), AncBE4max 689 (SEQ ID NO: 3706), and AncBE4max 687 (SEQ ID NO: 3707).

By “Cytidine Base Editor (CBE) polynucleotide” is meant a polynucleotide encoding a CBE. Non-limiting examples of polynucleotide sequences encoding cytidine deaminase base editors include those encoding BE4max (SEQ ID NO: 3721), AncBE4max689 (SEQ ID NO: 3722), and AncBE4max687 (SEQ ID NO: 3723).

By “cytidine deaminase” or “cytosine deaminase” is meant a polypeptide or fragment thereof capable of deaminating cytidine or cytosine. In embodiments, the cytidine or cytosine is present in a polynucleotide. In one embodiment, the cytidine deaminase converts cytosine to uracil or 5-methylcytosine to thymine. The terms “cytidine deaminase” and “cytosine deaminase” are used interchangeably throughout the application. Petromyzon marinus cytosine deaminase 1 (PmCDA1) (SEQ ID NO: 13-14), Activation-induced cytidine deaminase (AICDA) (SEQ ID NOs: 15-21), and APOBEC (SEQ ID NOs: 12-61) are exemplary cytidine deaminases. Further exemplary cytidine deaminase (CDA) sequences are provided in the Sequence Listing as SEQ ID NOs: 62-66 and SEQ ID NOs: 67-189. Non-limiting examples of cytidine deaminases include those described in PCT/US20/16288, PCT/US2018/021878, 180802-021804/PCT, PCT/US2018/048969, PCT/US2016/058344, PCT/US2020/062428, and PCT/US2019/033848, the disclosures of which are incorporated herein by reference in their entireties for all purposes. Non-limiting examples of cytidine deaminase amino acid sequences include amino acid sequences for Rat APOBEC1 (SEQ ID NO: 3684), Human APOBEC1 (SEQ ID NO: 3685), Human APOBEC3 (SEQ ID NO: 3686), Human APOBEC3B (SEQ ID NO: 3687), Human APOBEC3G (SEQ ID NO: 3688), evoAPOBEC3A (eA3A) (SEQ ID NO: 3689), evoCDA (SEQ ID NO: 3690), evoAPOBECI (SEQ ID NO: 3691), YE1 (SEQ ID NO: 3692), YE2 (SEQ ID NO: 3693), YEE (SEQ ID NO: 3694), EE (SEQ ID NO: 3695), R33A (SEQ ID NO: 3696), R33A+K34A (SEQ ID NO: 3697), AALN (SEQ ID NO: 3698), FERNY (SEQ ID NO: 3699), evoFERNY (SEQ ID NO: 3700), APOBEC (SEQ ID NO: 3724), Anc686 APOBEC (SEQ ID NO: 3725), Human APOBEC-3G D316R_D317R (SEQ ID NO: 5726), Human APOBEC-3G chain A (SEQ ID NO: 3727), Human APOBEC3-G chain A D120R_D121R (SEQ ID NO: 3728), Mouse APOBEC3 (SEQ ID NO: 3729), Rat APOBEC3 (SEQ ID NO: 3730), Rhesus macaque APOBEC-3G (SEQ ID NO: 3731), Chimpanzee APOBEC-3G (SEQ ID NO: 3732), Green Monkey APOBEC-3G (SEQ ID NO: 3733), Human APOBEC-3G (SEQ ID NO: 3734), Human APOBEC-3F (SEQ ID NO: 3735), Human APOBEC-3B (SEQ ID NO: 3736), Rat APOBEC-3B (SEQ ID NO: 3737), Bovine APOBEC-3B (SEQ ID NO: 3738), Chimpanzee APOBEC-3B (SEQ ID NO: 3739), Gorilla APOBEC-3C (SEQ ID NO: 3740), Human APOBEC-3A (SEQ ID NO: 3741), Rhesus macaque APOBEC-3A (SEQ ID NO: 3742), Bovine APOBEC-3A (SEQ ID NO: 3743), Human APOBEC-3H (SEQ ID NO: 3744), Human APOBEC-3D (SEQ ID NO: 3745), Rat ABOPEC1 (SEQ ID NO: 3746), Anc689 APOBEC (SEQ ID NO: 3747), Anc687 APOBEC (SEQ ID NO: 3748), Anc686 APOBEC (SEQ ID NO: 3749), Anc655 APOBEC (SEQ ID NO: 3750), and Anc733 APOBEC (SEQ ID NO: 3751).

By “cytidine deaminase polynucleotide” is meant a polynucleotide encoding a cytidine deaminase. Non-limiting examples of polynucleotide sequences encoding cytidine deaminase domains include those encoding Rat APOBEC1 (SEQ ID NO: 3709), Anc689 APOBEC (SEQ ID NO: 3710), Anc687 APOBEC (SEQ ID NO: 3711), Anc686 APOBEC (SEQ ID NO: 3712), Anc655 APOBEC (SEQ ID NO: 3713), Anc733 APOBEC (SEQ ID NO: 3714), Rat APOBEC1 (SEQ ID NO: 3715), Anc689 APOBEC (SEQ ID NO: 3716), Anc687 APOBEC (SEQ ID NO: 3717), Anc686 APOBEC (SEQ ID NO: 3718), Anc655 APOBEC (SEQ ID NO: 3719), and Anc733 APOBEC (SEQ ID NO: 3720).

By “cytosine deaminase activity” is meant catalyzing the deamination of cytosine or cytidine. In one embodiment, a polypeptide having cytosine deaminase activity converts an amino group to a carbonyl group. In an embodiment, a cytosine deaminase converts cytosine to uracil (i.e., C to U) or 5-methylcytosine to thymine (i.e., 5mC to T). In some embodiments, a cytosine deaminase as provided herein has increased cytosine deaminase activity (e.g., at least 10-fold, 20-fold, 30-fold, 40-fold, 50-fold, 60-fold, 70-fold, 80-fold, 90-fold, 100-fold or more) relative to a reference cytosine deaminase.

The term “deaminase” or “deaminase domain,” as used herein, refers to a protein or fragment thereof that catalyzes a deamination reaction.

The term “detect” refers to identifying the presence, absence or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. Exemplary diseases include diseases amenable to treatment with involving introducing an alteration to a complement complement factor B (CFB) polynucleotide in a cell that results in a reduction in activity and/or expression of a CFB polypeptide in the cell. In some instances, the disease is a disease associated with inappropriate activation of the complement system in the subject. Non-limiting examples of diseases associated with inappropriate activation of the complement system include blood disorders, transplant or graft rejection, inflammatory diseases or disorders, eye diseases or disorders, kidney diseases or disorders, heart disorders, respiratory diseases or disorders, autoimmune disorders, inflammatory bowel diseases or disorders, arthritis, neurodegenerative diseases or disorders, musculoskeletal diseases or disorders associated with inflammation, disorders affecting the integumentary system, diseases or disorders affecting the central nervous system, diseases or disorders affecting the circulatory system, diseases or disorders affecting the gastrointestinal system, diseases or disorders affecting the thyroid, chronic pain, allergies, and pulmonary diseases. Further non-limiting examples of diseases associated with inappropriate activation of the complement system include paroxysmal nocturnal hemoglobinuria (PNH), atypical hemolytic syndrome (aHUS), HELLP syndrome, autoimmune hemolytic anemia, transplant rejection, ischemia/reperfusion injury, transplant damage, hyperacute rejection, graft rejection or failure, acute antibody-mediated rejection, chronic inflammation, chronic allograft vasculopathy, chronic rejection of a transplant or graft, age-related macular degeneration (e.g., wet or dry age-related macular degeneration), diabetic retinopathy, glaucoma, uveitis, autoimmune diseases, myasthenia gravis, neuromyelitis optica (NMO), renal disease, membranoproliferative glomerulonephritis (MPGN) (e.g., MPGN type I, type II, or type III), IgA nephropathy (IgAN), primary membranous nephropathy, C3 glomerulopathy, proteinuria, a neurodegenerative disease, neuropathic pain, rhinosinusitis, nasal polyposis, cancer, sepsis, respiratory distress syndrome, anaphylaxis, infusion reaction, a respiratory disease or disorder (e.g., asthma or chronic obstructive pulmonary disease (COPD), oridiopathic pulmonary fibrosis, or asthma), a Th2-associated disorder (e.g., a disorder associated with high levels or high activation of CD4+ helper T cells of the Th2 subtype), a disorder associated with high levels or inappropriate activity of CD4+ helper T cells of the Th17 subtype, inflammatory bowel disease (e.g., Crohn's disease or ulcerative colitis), inflammatory skin diseases, a chronic inflammatory disease, psoriasis, atopic dermatitis, systemic scleroderma, sclerosis, Bechet's disease, dermatomyositis, polymyositis, multiple sclerosis (MS), dermatitis, meningitis, encephalitis, uveitis, osteoarthritis, lupus nephritis, rheumatoid arthritis (RA), Sjoren's syndrome, vasculitis, central nervous system (CNS) inflammatory disorders, chronic hepatitis, chronic pancreatitis, glomerulonephritis, sarcoidosis, thyroiditis, pathologic immune responses to tissue/organ transplantation, bronchiolitis, hypersensitivity pneumonitis, idiopathic pulmonary fibrosis (IPF), periodontitis, gingivitis, a disorder associated with excessive or inappropriate activity of IgE-producing cells, neuromyelitis optica, pemphigoid, pulmonary fibrosis (e.g., idiopathic pulmonary fibrosis), radiation-induced lung injury, allergic bronchopulmonary aspergillosis, hypersensitivity pneumonitis, eosinophilic pneumonia, interstitial pneumonia, sarcoid, Wegener's granulomatosis, bronchiolitis obliterans, allergic rhinitis, an inflammatory joint condition (e.g., arthritis such as rheumatoid arthritis or psoriatic arthritis, juvenile chronic arthritis, spondyloarthropathies Reiter's syndrome, or gout), a dermatomyositis, polymyositis, chronic muscle inflammation, pemphigus, systemic lupus erythematosus, dermatomyositis, scleroderma, sclerodermatomyositis, Sjögren syndrome, chronic urticaria, a demyelinating disease, amyotrophic lateral sclerosis, chronic pain, stroke, allergic neuritis, Huntington's disease, Alzheimer's disease, Parkinson's disease, a disease of the circulatory system, polyarteritis nodosa, Wegener's granulomatosis, giant cell arteritis, Churg-Strauss syndrome, microscopic polyangiitis, Henoch-Schonlein purpura, Takayasu's arteritis, Kawasaki disease, Behcet's disease, ulcerative colitis, thyroiditis (e.g., Hashimoto's thyroiditis, Graves' disease, post-partum thyroiditis), myocarditis, hepatitis (e.g., hepatitis C), pancreatitis, glomerulonephritis (e.g., membranoproliferative glomerulonephritis or membranous glomerulonephritis), panniculitis, eye disorders, choroidal neovascularization (CNV), retinal neovascularization (RNV), ocular inflammation, retinopathy of prematurity, proliferative vitreoretinopathy, uveitis, keratitis, conjunctivitis, and scleritis, geographic atrophy, conjunctivitis, keratitis, scleritis, iritis, iridocyclitis, cyclitis, pars planitis, choroiditis, persistent asthma, and allergic asthma. In some cases, the disease is selected from glaucoma, diabetic retinopathy, age-related macular degeneration, and neurological diseases such as amyotrophic lateral sclerosis (ALS), multiple sclerosis (MS), Alzheimer's disease, and various Tauopathies.

By “dual editing activity” or “dual deaminase activity” is meant having adenosine deaminase and cytidine deaminase activity. In one embodiment, a base editor having dual editing activity has both A→G and C→T activity, wherein the two activities are approximately equal or are within about 10% or 20% of each other. In another embodiment, a dual editor has A→G activity that no more than about 10% or 20% greater than C→T activity. In another embodiment, a dual editor has A→G activity that is no more than about 10% or 20% less than C→T activity. In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity. Non-limiting examples of proteins having dual deaminase activity include those described in International Patent Application Publications No. WO 2024/040083 and WO 2022/204574, the disclosures of which are hereby incorporated by reference in their entireties for all purposes.

By “effective amount” is meant the amount of an agent (e.g., a base editor, cell) as described herein, that is required to ameliorate the symptoms of a disease relative to an untreated patient or an individual without disease, i.e., a healthy individual, or is the amount of the agent sufficient to elicit a desired biological response. The effective amount of active compound(s) used to practice embodiments of the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the disclosure sufficient to introduce an alteration in a gene of interest in a cell (e.g., a cell in vitro or in vivo). In one embodiment, an effective amount is the amount of a base editor required to achieve a therapeutic effect. Such therapeutic effect need not be sufficient to alter a pathogenic gene in all cells of a subject, tissue or organ, but only to alter the pathogenic gene in about 1%, 5%, 10%, 25%, 50%, 75% or more of the cells present in a subject, tissue or organ. In one embodiment, an effective amount is sufficient to ameliorate one or more symptoms of a disease.

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In some embodiments, the fragment is a functional fragment.

By “guide polynucleotide” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inappropriate activation” in the context of factor B is meant any increase in complement activation that is associated with a disease or disorder. In an embodiment, inappropriate activation is activation that is increased or elevated locally (e.g., in an organ or tissue, such as in the central nervous system or in an eye) or systemically relative to a healthy reference (e.g., a healthy subject). In some instances “inappropriate activation” is activation that is associated with chronic (e.g., lasting more than 1, 2, 3, 4, 5, 6, 7, 8, 9, 10, 11, 12, 13, 14, or 15 weeks) inflammation in a subject. In some cases, inappropriate activation is activation that is directed against a tissue, cell, or organ of a subject and/or that leads to undesired damage to the tissue, cell, or organ of the subject. In embodiments, a disease or disorder associated with inappropriate activation of the complement system can be treated by any of the methods or compositions provided herein for reducing or eliminating expression and/or activity of a factor B polypeptide. In an embodiment, complement activation is detected by measuring levels of a factor B polypeptide and/or of a cleaved factor B polypeptide (e.g., a Ba fragment or a Bb fragment), where inappropriate activation can be determined as high levels of the factor B polypeptide and/or cleaved factor B polypeptide relative to a healthy reference subject.

By “increases” is meant a positive alteration of at least 10%, 25%, 50%, 75%, or 100%, or about 1.5 fold, about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The terms “inhibitor of base repair”, “base repair inhibitor”, “IBR” or their grammatical equivalents refer to a protein that is capable in inhibiting the activity of a nucleic acid repair enzyme, for example a base excision repair enzyme.

An “intein” is a fragment of a protein that is able to excise itself and join the remaining fragments (the exteins) with a peptide bond in a process known as protein splicing. The process of an intein excising itself and joining the remaining portions of the protein is herein termed “protein splicing” or “intein-mediated protein splicing.” In some embodiments, an intein is a trans-splicing intein (also referred to as a “split intein”). In the case of trans-splicing inteins, a full-length polypeptide is split into two separate fragments and the C-terminus of the N-terminal fragment is fused to an N-terminal fragment of a split intein intein (N-intein) and the N-terminus of the remaining C-terminal fragment is fused a C-terminal fragment of a split intein (C-intein). Not intending to be bound by theory or mechanism of action, contacting the two polypeptide sequences with one another results in excision of the intein and joining of the two polypeptide sequences together to form a full-length polypeptide sequence. In embodiments, contacting the two polypeptide fragments each fused to an intein fragment, or peptide derived from an intein fragment, is associated with a measured catalytic activity (e.g., deamination of a nucleobase in a polynucleotide sequence) in a cell that is greater than that observed when the two polypeptide fragments are contacted with one another in a cell and do not contain any intein fragments. Non-limiting examples of N-intein and C-intein sequences include those sequences sharing at least 85% sequence identity to an amino acid sequence listed in Table A or Table B, or functional fragments thereof.

TABLE A
Representative synthetic N-intein
amino acid sequences.

| | | SEQ |
|---|---|---|
| | | ID |
| Intein | Sequence | NO |
| Syn2-N | CLSYDTEILTVEYGLIPIGEIVEKKIECTVYTIDNNGLI | 3560 |
| | YTQSIEQWHHRGYQELFEYILEDGSTIRATKDHKFMTSE | |
| | RQMLPIEEIFERGWELKQVL | |
| Syn3-N | CLSSDTEVITEEYGPIAIGKIVDEGIRCSVYSVDNNGNL | 3561 |
| | YTQPISQWHDRGROEIYEYYLENGSVIRATKDHKFMTKD | |
| | GEMLPIDEIFEKGLELKQVLP | |
| Syn5-N | CLSYETEVLTVEYGFMPIGKIVEERIRCSVYTVDKNGFI | 3562 |
| | YSQPIAQWHQRGLQEVYEYDLENGSIIRATKEHQFMIND | |
| | GQMLAIHEIFTRKLDLLQSQE | |

TABLE B
Representative synthetic C-intein
amino acid sequences.

| | | SEQ |
|---|---|---|
| | | ID |
| Intein | Sequence | NO |
| Syn1-C | MKVISRKSLGTQPVYDICVTHDHNFLMKNGLIASN | 3563 |
| Syn4-C | MDVKIVSYKFLGSENVYDILERDHNFLIKNGLVASN | 3564 |
| Syn5-C | MVKIITYKSLGRQKVYDLGLEQDHNFVLANGLVASN | 3565 |
| Syn9-C | MVKIISRKYLDTQPVYDVGVQKDHNFLISNGSIASN | 3566 |
| Syn10-C | MVKIATRRSLGTEPVYDIGLQQEHNFLLANGLVASN | 3567 |

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “linker”, as used herein, refers to a molecule that links two moieties. In one embodiment, the term “linker” refers to a covalent linker (e.g., covalent bond) or a non-covalent linker.

By “marker” is meant any protein or polynucleotide having an alteration in expression, level, structure, or activity that is associated with a disease or disorder. In embodiments, the disease or disorder is associated with inappropriate activation of the complement system. In some cases, the marker is a factor B polynucleotide or polypeptide.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, IRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi: 10.1038/nbt.4172. In some embodiments, an NLS comprises the amino acid sequence KRTADGSEFESPKKKRKV (SEQ ID NO: 190), KRPAATKKAGQAKKKK (SEQ ID NO: 191), KKTELQTTNAENKTKKL (SEQ ID NO: 192), KRGINDRNFWRGENGRKTR (SEQ ID NO: 193), RKSGKIAAIVVKRPRK (SEQ ID NO: 194), PKKKRKV (SEQ ID NO: 195), MDSLLMNRRKFLYQFKNVRWAKGRRETYLC (SEQ ID NO: 196), PKKKRKVEGADKRTADGSEFESPKKKRKV (SEQ ID NO: 328), or RKSGKIAAIVVKRPRKPKKKRKV (SEQ ID NO: 329).

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Ψ). A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpf1, Cas12b/C2c1, Cas12c/C2c3, Cas12d/CasY, Cas12e/CasX, Cas12g, Cas12h, Cas12i, and Cas12j/Cas@ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Cas12j/CasΦ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363 (6422): 88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 197-231, 232-245, 254-257, 260, and 378. In some embodiments, the napDNAbp is a (CRISPR-associated system) Cas9 endonuclease, for example, Cas9 (Csnl) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9), or derivatives thereof (e.g., a sequence with at least about 85% sequence identity to a Cas9, such as Nme2Cas9 or spCas9). Further non-limiting examples of nucleic acid programmable DNA binding proteins include those disclosed or referenced in Rufflow, et al., “Design of highly functional genome editors by modeling of the universe of CRISPR-Cas Sequences,” bioRxiv, posted Apr. 22, 2024, doi: 10.1101/2024.04.22.590591, the disclosure of which is incorporated herein by reference in its entirety for all purposes, which were designed using artificial intelligence. In some embodiments, the napDNAbp is OpenCRISPR-1, or a variant thereof (e.g., a variant comprising a D10A amino acid alteration and/or lacking an N-terminal methionine). Further non-limiting examples of nucleic acid programmable DNA binding proteins include those disclosed in International Patent Application No. PCT/US2019/047996.

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase).

As used herein, “obtaining” as in “obtaining an agent” includes synthesizing, purchasing, or otherwise acquiring the agent.

By “OpenCRISPR-1 polypeptide” is meant a protein with an amino acid sequence having at least about 85% amino acid sequence identity to SEQ ID NO: 3568, or a fragment thereof that associates with a nucleic acid, such as a guide nucleic acid or guide polynucleotide, that guides the napDNAbp to a specific nucleic acid sequence. Further details relating to the OpenCRISPR-1 polypeptide are disclosed in Rufflow, et al., “Design of highly functional genome editors by modeling of the universe of CRISPR-Cas Sequences,” bioRxiv, posted Apr. 22, 2024, doi: 10.1101/2024.04.22.590591, the disclosure of which is incorporated herein by reference in its entirety for all purposes.

By “OpenCRISPR-1 polynucleotide” is meant a nucleic acid molecule encoding an OpenCRISPR-1 polypeptide, as well as the introns, exons, 3′ untranslated regions, 5′ untranslated regions, and regulatory sequences associated with its expression, or fragments thereof. In embodiments, an OpenCRISPR-1 polynucleotide is the genomic sequence, cDNA, mRNA, or gene associated with and/or required for OpenCRISPR-1 expression. An exemplary OpenCRISPR-1 nucleotide sequence is provided at SEQ ID NO: 3569.

In various embodiments, a guide RNA suitable for use in combination with an OpenCRISPR-1 polypeptide contains a scaffold having at least 85% sequence identity to a nucleotide sequence selected from the following, or fragments thereof capable of binding to an OpenCRISPR-1 polypeptide:

(SEQ ID NO: 3570)
GUUUUAGAGCUGUGUUGAAAAACACAGCAAGUUAAAAUAAGGCUUUGUCC
GUAUCCAACUUGAAAAAGUGAGCACCGAUUCGGUGC;
(SEQ ID NO: 3571)
GUUUUAGAGCUGGAAACAGCAAGUUAAAAUAAGGCUUUGUCCGUAUCCAA
CUUGAAAAAGUGAGCACCGAUUCGGUGC;
and
(SEQ ID NO: 3572)
GUUUUAGAGCUAGAAAUAGCAAGUUAAAAUAAGGCUAGUCCGUUAUCAAC
UUGAAAAAGUGGCACCGAGUCGGUGC.

By “subject” or “patient” is meant a mammal, including, but not limited to, a human or non-human mammal. In embodiments, the mammal is a bovine, equine, canine, ovine, rabbit, rodent, nonhuman primate, or feline. In an embodiment, “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder.

The terms “pathogenic mutation”, “pathogenic variant”, “disease causing mutation”, “disease causing variant”, “deleterious mutation”, or “predisposing mutation” refers to a genetic alteration or mutation that is associated with a disease or disorder or that increases an individual's susceptibility or predisposition to a certain disease or disorder. In some embodiments, the pathogenic mutation comprises at least one wild-type amino acid substituted by at least one pathogenic amino acid in a protein encoded by a gene.

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

The term “recombinant” as used herein in the context of proteins or nucleic acids refers to proteins or nucleic acids that do not occur in nature but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%.

By “reference” is meant a standard or control condition. In one embodiment, the reference is a wild-type or healthy cell. In other embodiments and without limitation, a reference is an untreated cell that is not subjected to a test condition, or is subjected to placebo or normal saline, medium, buffer, and/or a control vector that does not harbor a polynucleotide of interest. In embodiments, a reference is a healthy subject or cell without inappropriate activation of the complement system. In some cases, a reference is an unedited or untreated cell (e.g., a hepatocyte), tissue (e.g., component of the central nervous system or an organ, such as a liver, eye) and/or subject. In embodiments, a reference is a subject not administered a composition of the disclosure or a component thereof. In some cases, a reference is a subject prior to a change in treatment.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” refer to a nuclease that forms a complex with (e.g., binds or associates with) one or more RNA(s) that is not a target for cleavage. In some embodiments, an RNA-programmable nuclease, when in a complex with an RNA, may be referred to as a nuclease-RNA complex. Typically, the bound RNA(s) is referred to as a guide RNA (gRNA). In some embodiments, the RNA-programmable nuclease is the (CRISPR-associated system) Cas9 endonuclease, for example, Cas9 (Csnl) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9), or derivatives thereof (e.g., a sequence with at least about 85% sequence identity to a Cas9, such as Nme2Cas9 or spCas9).

By “specifically binds” is meant a nucleic acid molecule, polypeptide, polypeptide/polynucleotide complex, compound, or molecule that recognizes and binds a polypeptide and/or nucleic acid molecule of the disclosure, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least about 60%, 80%, 85%, 90%, 95%, 96%, 97%, 98%, 99%, 99.9%, or even 99.99%, identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “split” is meant divided into two or more fragments.

A “split polypeptide” or “split protein” refers to a protein that is provided as an N-terminal fragment and a C-terminal fragment translated as two separate polypeptides from a nucleotide sequence(s). The polypeptides corresponding to the N-terminal portion and the C-terminal portion of the split protein may be spliced in some embodiments to form a “reconstituted” protein. In embodiments, the split polypeptide is a nucleic acid programmable DNA binding protein (e.g. a Cas9) or a base editor.

The term “target site” refers to a nucleotide sequence or nucleobase of interest within a nucleic acid molecule that is modified. In embodiments, the modification is deamination of a base. The deaminase can be a cytidine or an adenine deaminase. The fusion protein or base editing complex comprising a deaminase may comprise a dCas9-adenosine deaminase fusion protein, a Cas12b-adenosine deaminase fusion, or a base editor disclosed herein.

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, reduces the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a composition as described herein.

By “uracil glycosylase inhibitor” or “UGI” is meant an agent that inhibits the uracil-excision repair system. Base editors comprising a cytidine deaminase convert cytosine to uracil, which is then converted to thymine through DNA replication or repair. In various embodiments, a uracil DNA glycosylase (UGI) prevent base excision repair which changes the U back to a C. In some instances, contacting a cell and/or polynucleotide with a UGI and a base editor prevents base excision repair which changes the U back to a C. An exemplary UGI comprises an amino acid sequence as follows:

>splP14739IUNGI BPPB2 Uracil-DNA
glycosylase inhibitor
(SEQ ID NO: 231)
MTNLSDIIEKETGKQLVIQESILMLPEEVEEVIGNKPESDILVHTAYDES
TDENVMLLTSDAPEYKPWALVIQDSNGENKIKML.

In some embodiments, the agent inhibiting the uracil-excision repair system is a uracil stabilizing protein (USP). See, e.g., WO 2022015969 A1, incorporated herein by reference.

As used herein, the term “vector” refers to a means of introducing a nucleic acid molecule into a cell, resulting in a transformed cell. Vectors include plasmids, transposons, phages, viruses, liposomes, lipid nanoparticles, and episomes.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include,” “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended. This wording indicates that specified elements, features, components, and/or method steps are present, but does not exclude the presence of other elements, features, components, and/or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIG. 1 provides a schematic diagram depicting the alternative pathway of complement amplification.

FIG. 2 provides a bar graph showing maximum percent A to G base editing of a factor B polynucleotide measured in HEK293T cells transfected with base editor systems containing an adenosine deaminase and the guides indicated along the x-axis. A base editor system containing the guide sg23 and an adenosine deaminase was used as a positive control for base editing.

FIG. 3 provides a bar graph showing maximum percent C to T base editing of a factor B polynucleotide measured in HEK293T cells transfected with base editor systems containing a cytidine deaminase and the guides indicated along the x-axis. A base editor system containing the guide sg23 and a cytidine deaminase was used as a positive control for base editing.

FIG. 4 provides a bar graph showing maximum percent A to G base editing of a factor B polynucleotide measured in HEK293T cells transfected with base editor systems containing the indicated adenosine deaminases and guide polynucleotides. In FIG. 4, the term “NHP X-Reactivity” means “non-human primate cross-reactivity.” A base editor system with NHP X-Reactivity will edit both a human a non-human primate factor B polynucleotide. Each set of bars, from left-to-right, correspond to base editor systems containing the following guide polynucleotides, respectively: gRNA1193, gRNA1120, gRNA1230, gRNA1217, gRNA1204, gRNA1218, gRNA1203, gRNA1202, gRNA1190, gRNA1213, gRNA1210, and sg23. The guide polynucleotide sg23 was used as a positive control.

FIG. 5 provides a bar graph showing human complement factor B (hCFB) protein levels (left axis and left bar of each pair of bars) in primary human hepatocytes (PHH) at day 11 (D11) post transfection (P-TF) with the indicated base editor systems, and maximum percent A to G base editing (right axis and right bar of each pair of bars) of a factor B polynucleotide measured in the PHH at day 13 (D13) P-TF with the indicated base editor systems. In FIG. 5, the listed editors are base editors containing the indicated TadA* adenosine deaminase domain, and the term “NHP X-Reactivity” means “non-human primate cross-reactivity.” A base editor system with NHP X-Reactivity will edit both a human a non-human primate factor B polynucleotide. The guide sg23 was used as a positive control. The guide polynucleotide gRNA1204 targeted the human factor B polynucleotide sequence GCTTACAATGACTGAGATCTTGG (SEQ ID NO: 429), which differs from the following non-human primate (cyno) factor B polynucleotide sequence at the G in bold: GCTTACAGTGACTGAGATCTTGG (SEQ ID NO: 430). An Abcam Elisa Kit (Human Factor B ELISA Kit (ab137973)) was used to measure protein levels (Range: 4.375 ng/ml-140 ng/ml; lower limit of quantitation (LLOQ): 0.8 ng/mL). In FIG. 5, the editor “spCas9” refers to an spCas9 endonuclease capable of inducing a double-stranded break of DNA.

FIG. 6 provides a bar graph showing the impact of guide polynucleotide spacer length on percent A to G base editing of a factor B polynucleotide in HEK293T cells. The cells were base edited using base editor systems containing the indicated adenosine deaminase base editor and the guide RNA with a spacer having the indicated nucleotide (nt) length ranging from 19 to 23 nucleotides. The first 5 bars from the left correspond to the base editor ABE8.8 with specificity for an NGG PAM sequence, the second 5 bars from the left correspond to the base editor ABE 8.13 with specificity for an NGG PAM sequence, the third 5 bars from the left correspond to the base editor ABE 8.8 with specificity for an NGG PAM sequence, and the rightmost bar corresponds to ABE8.8 with specificity for an NGG PAM sequence.

FIGS. 7A and 7B provide a bar graph and a schematic diagram relating to optimization of guide spacer length. FIG. 7A provides a bar graph showing human complement factor B (hCFB) protein levels (left axis and left bar of each pair of bars) in human hepatocytes isolated from a PXB-mouse (PXB cells) at day 11 (D11) post transfection (P-TF) with the indicated base editor systems, and maximum percent A to G base editing (right axis and right bar of each pair of bars) of a factor B polynucleotide measured in the PXB cells at day 13 (D13) P-TF with the indicated base editor systems. FIG. 7A, the listed editors are base editors containing the indicated TadA* adenosine deaminase domain, the term “NHP X-Reactivity” means “non-human primate cross-reactivity,” and the term “Protospacer Length (nt)” indicates the length (19-23 nucleotides) of the spacer in nucleotides (nt) corresponding to the indicated guide polynucleotides. A base editor system with NHP X-Reactivity will edit both a human a non-human primate factor B polynucleotide. The guide sg23 was used as a positive control. The guide polynucleotide gRNA1204 targeted the human factor B polynucleotide sequence GCTTACAATGACTGAGATCTTGG (SEQ ID NO: 429), which differs from the following non-human primate (cyno) factor B polynucleotide sequence targeted by the guide polynucleotide gRNA1999 (gRNA1204 non-human primate surrogate) at the G in bold: GCTTACAGTGACTGAGATCTTGG (SEQ ID NO: 430). An Abcam Elisa Kit (Human Factor B ELISA Kit (ab137973)) was used to measure protein levels (Range: 4.375 ng/ml-140 ng/ml; lower limit of quantitation (LLOQ): 0.8 ng/ml). An Abcam Elisa Kit (Human Factor B ELISA Kit (ab137973)) was used to measure protein levels (Range: 4.375 ng/ml-140 ng/ml; lower limit of quantitation (LLOQ): 0.8 ng/mL). FIG. 7B provides a schematic diagram describing the experiment used to gather the data presented in FIG. 7A. In FIG. 7B, the term “NGS” indicates next-generation sequencing.

FIGS. 8A and 8B provide bar graphs and a Western blot image showing complement factor B polynucleotide base editing efficiency measured in primary cyno hepatocytes (PCH) transfected with base editor systems containing an adenosine deaminase and one of the indicated guides, which were either non-human primate and human factor B cross-reactive or hon-human primate surrogate guide polynucleotides. FIG. 8A provides a bar graph showing maximum percent A to G base editing of a factor B polynucleotide measured in PCH transfected with base editor systems containing an adenosine deaminase and the indicated guide polynucleotides. The guide polynucleotide gRNA2072 targeted the human factor B polynucleotide sequence GCTTACAATGACTGAGATCTTGG (SEQ ID NO: 429), which differs from the following non-human primate (cyno) factor B polynucleotide sequence at the G in bold: GCTTACAGTGACTGAGATCTTGG (SEQ ID NO: 430). The top panel of FIG. 8B provides a Western blot showing levels of factor B measured in monkey serum, PCH supernatant, humanized mice serum, and in an Abcam human complement factor B (CFB) ELISA standard using an anti-complement factor B monoclonal antibody (Ab-CFB). The lower panel of FIG. 8B provides a bar graph showing cyno CFB protein levels normalized to pre-treatment levels for cells corresponding to FIG. 8A.

FIGS. 9A-9D provide bar graphs and plots showing maximum percent A to G base editing of a factor B polynucleotide measured in primary human hepatocytes (PHH) or human hepatoma cells (HepG2 cells) transfected with base editor systems containing the indicated mRNAs encoding an adenosine deaminase and the indicated guide polynucleotides. The base editors encoded by the MRNA molecules referenced in the figures (e.g., m3534/MRNA3534) are described in Table 9. FIG. 9A provides a bar graph showing maximum percent A to G base editing of a factor B polynucleotide measured in PHH transfected with the indicated base editor systems. The base editor system sg23/m3534 was used as a positive control. FIGS. 9B-9D provide plots showing maximum percent A to G base editing of a factor B polynucleotide measured in HepG2 cells transfected with base editor systems containing different doses of the guide polynucleotides TSBTx3826, TSBTx3837, and TSBTx3935, respectively, and a constant dose of the indicated mRNA molecules encoding a base editor. The base editors encoded by the MRNA molecules referenced in the figures (e.g., MRNA3534) are described in Table 9.

FIGS. 10A and 10B provide bar graphs showing human complement factor B (hCFB) maximum percent A to G base editing, insertion/deletion (indel) mutation rates, and protein levels in FRG™ liver-humanized mice administered a base editor system containing the guide polynucleotide gRNA1193 and an ABE8.8 adenosine deaminase base editor. FIG. 10A provides a bar graph showing hCFB maximum percent A to G base editing and indel mutation rates measured in FRG™ liver-humanized mice transfected with a base editor system containing an adenosine deaminase base editor and 2 mg/kg (mpk) or 0.3 mpk of the end-modified guide polynucleotide gRNA1193. The mice were administered tris buffered saline (TBS) as a negative control. FIG. 10B provides a bar graph showing concentrations (Conc.) of hCFB protein (hCFB Pr.) measured in FRG™ liver-humanized mice transfected with a base editor system containing an adenosine deaminase base editor and 2 mg/kg (mpk) of the end-modified guide polynucleotide gRNA1193. In FIG. 10B, each set of three bars corresponds, from left-to-right, to measurements taken at day 0 (DO) prior to transfection (i.e., “Predose”), at day 7 post-transfection, and at the end of the experiment (i.e., “Terminal”), which was day 14 post-transfection. The upper panel of FIG. 10B shows unnormalized protein concentrations and the lower panel of FIG. 10B shows protein concentrations normalized to day 0 (DO) concentrations.

FIG. 11 provides a set of plots showing a negative correlation between serum hC3 and hCFB protein levels in FRG™ liver-humanized mice transfected with a base editor system containing an ABE8.8 adenosine deaminase base editor and 2 mg/kg (mpk) or 0.3 mpk of the end-modified guide polynucleotide gRNA1193. The x-axis indicates the day post-transfection at which measurements were taken. In FIG. 11, the arrows extending from each curve indicate the axis to which each curve corresponds. The mice were administered tris buffered saline (TBS) as a negative control.

FIGS. 12A and 12B provide bar graphs showing human complement factor B (hCFB) percent A to G base editing (FIG. 12A) and protein levels (FIG. 12B) in FRG™ liver-humanized mice administered a base editor system containing the guide polynucleotide TSBTx3826 with an NLS nucleotide modification scheme and one of the indicated adenosine deaminase base editors (i.e., ABE8.8, ABE8.20, or ABE9.52). A base editor system containing the guide polynucleotide sg23 was used as a positive control. In FIGS. 12A and 12B the term “Mod Schem selection” indicates the nucleotide modification scheme of the guide polynucleotide, the term “BE selection (NLS)” indicates base editor selection using guide polynucleotides having an NLS nucleotide modification scheme, the term “Pre-dose” indicates a measurement taken prior to administration of the base editor system to the mice, and the terms “0.5 mpk” and “0.3 mpk” indicate the dose of guide polynucleotide administered to the mice. The TSBTx3826 guide polynucleotide was cross-reactive (i.e., targeted for base editing) both human and cyno CFB polynucleotides, and the location of the target base edit was a splice site at the 5′-end of Exon 3 of the factor B polynucleotide. In FIGS. 12A and 12B, the term “ABE9.52” refers to a base editor containing the following adenosine deaminase domain: TadA*8.20 with the amino acid alterations V82T, Y147T, and Q154S.

FIG. 13 provides a bar graph showing levels of the indicated human complement factor B (hCFB) exons in mRNA collected from tissues of FRG™ liver-humanized mice administered a base editor system containing the guide polynucleotide TSBTx3826 targeting a splice site at the 5′-end of Exon 10 and one of the indicated adenosine deaminase base editors. Measurements were taken at day 14 post-administration of the base editor system. In FIG. 13, mRNA levels were normalized to mRNA levels measured for an actin beta (ACTB) gene. In FIG. 13, the term “ALAS1 (sg23)” indicates levels of 5′-Aminolevulinate Synthase 1 (ALAS1) transcripts in mice administered a base editor system containing the guide polynucleotide sg23. In FIG. 13, the term “ABE9.52” refers to a base editor containing the following adenosine deaminase domain: TadA*8.20 with the amino acid alterations V82T, Y147T, and Q154S.

FIGS. 14A and 14B provide bar graphs showing human complement factor B (hCFB) percent A to G base editing (FIG. 14A) and protein levels (FIG. 14B) in FRG™ liver-humanized mice administered a base editor system containing the guide polynucleotide TSBTx3837 with an HM01 nucleotide modification scheme and one of the indicated adenosine deaminase base editors (i.e., ABE8.8 or ABE8.20). Base editor systems containing the guide polynucleotide sg23 or TSBTx3826 having an NLS nucleotide modification scheme were used as controls. In FIGS. 14A and 14B the term “Mod Schem selection” indicates the nucleotide modification scheme of the guide polynucleotide, the term “BE selection (NLS)” indicates base editor selection using guide polynucleotides having an NLS nucleotide modification scheme, the term “Pre-dose” indicates a measurement taken prior to administration of the base editor system to the mice, and the terms “0.5 mpk” and “0.3 mpk” indicate the dose of guide polynucleotide administered to the mice. The TSBTx3837 guide polynucleotide targeted hCFB and was not cross-reactive (i.e., targeted for base editing) cyno CFB polynucleotides because the TSBTx3837 guide polynucleotide target site differed from the corresponding cyno CFB target site by one (1) nucleotide, and the location of the target base edit was a splice site at the 3′-end of Exon 11 of the factor B polynucleotide. In FIGS. 14A and 14B, the term “ABE9.52” refers to a base editor containing the following adenosine deaminase domain: TadA*8.20 with the amino acid alterations V82T, Y147T, and Q154S.

FIG. 15 provides a bar graph showing levels of the indicated human complement factor B (hCFB) exons in mRNA collected from tissues of FRG™ liver-humanized mice administered a base editor system containing the guide polynucleotide TSBTx3837 targeting a splice site at the 3′-end of Exon 11 and one of the indicated adenosine deaminase base editors. Measurements were taken at day 14 post-administration of the base editor system. In FIG. 15, mRNA levels were normalized to mRNA levels measured for an actin beta (ACTB) gene. In FIG. 15, the term “ALAS1 (sg23)” indicates levels of 5′-Aminolevulinate Synthase 1 (ALAS1) transcripts in mice administered a base editor system containing the guide polynucleotide sg23.

FIGS. 16A and 16B provide bar graphs showing human complement factor B (hCFB) percent A to G base editing (FIG. 16A) and protein levels (FIG. 16B) in FRG™ liver-humanized mice administered a base editor system containing the guide polynucleotide TSBTx3835 with an end-mod nucleotide modification scheme and one of the indicated adenosine deaminase base editors (i.e., ABE8.13 or ABE9.52). Base editor systems containing the guide polynucleotide sg23 or TSBTx3826 having an NLS nucleotide modification scheme were used as controls. In FIGS. 16A and 16B the term “Mod Schem selection” indicates the nucleotide modification scheme of the guide polynucleotide, the term “BE selection (NLS)” indicates base editor selection using guide polynucleotides having an NLS nucleotide modification scheme, the term “Pre-dose” indicates a measurement taken prior to administration of the base editor system to the mice, and the terms “0.5 mpk” and “0.3 mpk” indicate the dose of guide polynucleotide administered to the mice. The TSBT×3835 guide polynucleotide targeted hCFB and was cross-reactive (i.e., targeted for base editing) with human and cyno CFB polynucleotides, and the location of the target base edit was a splice site at the 3′-end of Exon 16 of the factor B polynucleotide. In FIGS. 16A and 16B, the term “ABE9.52” refers to a base editor containing the following adenosine deaminase domain: TadA*8.20 with the amino acid alterations V82T, Y147T, and Q154S.

FIG. 17 provides a bar graph showing levels of the indicated human complement factor B (hCFB) exons in mRNA collected from tissues of FRG™ liver-humanized mice administered a base editor system containing the guide polynucleotide TSBTx3835 targeting a splice site at the 3′-end of Exon 16 and one of the indicated adenosine deaminase base editors. Measurements were taken at day 14 post-administration of the base editor system. In FIG. 17, mRNA levels were normalized to mRNA levels measured for an actin beta (ACTB) gene. In FIG. 17, the term “ALAS1 (sg23)” indicates levels of 5′-Aminolevulinate Synthase 1 (ALAS1) transcripts in mice administered a base editor system containing the guide polynucleotide sg23. In FIG. 17, the term “ABE9.52” refers to a base editor containing the following adenosine deaminase domain: TadA*8.20 with the amino acid alterations V82T, Y147T, and Q154S.

FIG. 18 provides a plot showing complement factor B polynucleotide maximum percent A to G editing in primary human hepatocytes (PHH) or primary cyno hepatocytes (PCH), as indicated, transfected with base editor systems containing the indicated guide polynucleotides and an adenosine deaminase base editor. A base editor system containing an adenosine deaminase and the guide polynucleotide sg23 was used as a positive control. Cells were transfected with the guide polynucleotide and mRNA encoding the base editor at a mass ratio of 1-to-3 (1:3). The TSBTx3837 guide was used in combination with the base editor ABE8.20, and the guide had an HM01 nucleotide modification scheme. The TSBTx3826 guide was used in combination with a base editor containing a TadA*8.20 adenosine deaminase domain with the amino acid alterations V82T, Y147T, and Q154S, and the guide had an NLS nucleotide modification scheme.

FIGS. 19A-19D provide a schematic diagram and plots. FIG. 19A provides a schematic diagram showing the sequence of a polynucleotide construct used to compare the potency of guide polynucleotides targeting human complement factor B (CFB) and/or non-human primate CFB for base editing. The binding sites for guide polynucleotides targeting a human CFB polynucleotide (i.e., “CFB guide-human”) and a non-human primate CFB polynucleotide (i.e., “CFB guide-NHP”) are indicated. In FIG. 19A, the term “10 bp” indicates a 10 nucleotide spacer, and the term “30 bp random spacer” indicates a randomized sequence of 30 nucleotides. In FIG. 19A, the two nucleotide sequences depicted are reverse complements of one another. In FIG. 19A, the upper nucleotide sequence is. CATGGCAGGCCAAGATCTCAGTCATTGTAAGCACAGAATCCCATATGGAAGGTCATTAGCTC CGGCAAGCAATCATGGCAGGCCAAGATCTCAGTCACTGTAAGCACAGAATCCCA (SEQ ID NO: 431), and the amino acid sequences are HGRPRSQSL (SEQ ID NO: 432) and AQNPIWKVISSGKQSWQAKISVTVSTES (SEQ ID NO: 433). The term “*” in the amino acid sequence of FIG. 19A indicates a stop codon, and the term “CFB insert” indicates that the polynucleotide construct was inserted into the genome of HEK293T cells. FIGS. 19B-19D show percent base editing in three separate experiments (i.e., Batch 1, Batch 2, and Batch 3, respectively) at the “CFB guide-human” and “CFB guide-NHP” sites in HEK293T cells transfected with base editor systems containing an adenosine deaminase and the indicated doses of the guide gRNA2067 (TSBTx3837; targeting the CFB guide-human site) or the guide gRNA2072 (TSBTx2072; targeting the CFB guide-HNP site).

FIG. 20 provides a bar graph showing complement factor B (CFB) TATA box A to G editing in human hepatoma cells (HepG2 cells) transfected with the indicated base editor systems (i.e., Sample 1 to Sample 16, which are described in Table 12.1A) containing a guide polynucleotide and an adenosine deaminase. The cells were transfected with a saturating dose of 800 ng total of guide polynucleotide and mRNA encoding the base editor at a mass ratio of 1:3. The CFB TATA box was located at positions −157 to −151 relative to the CFB start codon. A base editor system containing an adenosine deaminase base editor and the guide sgRNA_088 (sg23) was used as a positive control. The bars of FIG. 20 each correspond in order, from left-to-right, to base editor systems containing the base editors listed in Table 12.1A.

FIGS. 21A and 21B provide bar graphs showing complement factor B (CFB) start codon A to G editing in human hepatoma cells (HepG2 cells) (FIG. 21A) or primary human hepatocyte (PHH) monolayer cells transfected with base editor systems (i.e., Sample 1 to Sample 8 of FIG. 21A and Sample 1 to Sample 3 of FIG. 21B, which are described in Table 12.1B) containing a guide polynucleotides and an adenosine deaminase. A base editor system containing an adenosine deaminase base editor and the guide sgRNA_088 (sg23) was used as a positive control. Beneath the x-axis of the bar graphs of FIGS. 21A and 21B are listed the CFB amino acid alterations (e.g., MIT, G2E, G2R, L5P, or S3P) corresponding to the base edits corresponding to each bar. The cells were transfected with a saturating dose of 800 ng total of guide polynucleotide and mRNA encoding the base editor at a mass ratio of 1:3.

FIGS. 22A and 22B provide a bar graph and a schematic diagram relating to complement factor B (CFB) TATA-box and start codon disruption in primary human hepatocytes (PHH) for protein knock-down. FIG. 22A provides a bar graph showing human complement factor B (hCFB) protein levels (left axis and left bar of each pair of bars) in PHH at day 12 (D12) post transfection (P-TF) with base editor systems (i.e., Sample 1 to Sample 16, which are described in Table 12.1C) containing an adenosine deaminase and a guide polynucleotide, and maximum percent A to G base editing (right axis and right bar of each pair of bars) of a factor B polynucleotide measured in the PXB cells at day 13 (D13) P-TF with the base editor systems. FIG. 22A, a base editor system containing an adenosine deaminase and the guide polynucleotide sgRNA_088 (sg23) was used as a positive control for base editing. FIG. 22B provides a schematic diagram describing the experiment used to gather the data presented in FIG. 22A. In FIG. 22B, the term “MC” indicates a media change, and the term “NGS” indicates next-generation sequencing. The data of FIG. 22A is not normalized; however, a similar pattern was observed with data normalization to protein levels prior to transfection (i.e., day 0).

FIG. 23 provides a set of bar graphs showing high editing and good reduction of complement factor B (CFB) protein levels in primary human hepatocyte (PHH) co-cultures transfected with base editor systems containing an adenosine deaminase with one of the indicated PAM specificities (e.g., NGC, NGG, or NGA) and one of the indicated guide polynucleotides targeting the CFB start codon for base editing. Base editor systems containing an adenosine deaminase and the guide polynucleotide sg23 or gRNA1193 (TSBTx3826) were used as a positive control. In FIG. 23, the term “dABE (−) Control” indicates a defective or “dead” adenosine base editor. The top panel of FIG. 23 presents data collected using cells from a donor designated “JGC” and the lower panel of FIG. 23 presents data collected using cells from a donor designated “MRW.” The base editor systems were administered to the cells at a saturating total dose of 800 ng of the guide polynucleotide and mRNA encoding the adenosine deaminase. None of the guide polynucleotides were cross-reactive with non-human primate target sites (i.e., the guides target a human CFB polynucleotide for base editing but not a cyno CFB polynucleotide). The target site for gRNA 3657 was TGCTCCCCATGGCGTTGGAAGGC (SEQ ID NO: 434), whereas the corresponding non-human primate (NHP) target site is TGCTCCCCATGGCATTAGAAGGC (SEQ ID NO: 435), where bold nucleotides indicate where the human gRNA 3657 target site differs from the corresponding NHP target site, and where the nucleotides corresponding to the CFB start codon are underlined. The target site for gRNA 3658 was TTGCTCCCCATGGCGTTGGAAGG (SEQ ID NO: 436), whereas the corresponding non-human primate (NHP) target site is CTGCTCCCCATGGCATTAGAAGG (SEQ ID NO: 437), where bold nucleotides indicate where the human gRNA 3658 target site differs from the corresponding NHP target site, and where the nucleotides corresponding to the CFB start codon are underlined. The target site for gRNA 3660 was CCCCATGGCGTTGGAAGGCAGGA (SEQ ID NO: 438), whereas the corresponding non-human primate (NHP) target site is CCCCATGGCATTAGAAGGCAGGA (SEQ ID NO: 439), where bold nucleotides indicate where the human gRNA 3660 target site differs from the corresponding NHP target site, and where the nucleotides corresponding to the CFB start codon are underlined. In FIG. 23, for every set of three bars, the first two bars from the left correspond to hCFB protein level measurements taken at day 7 (D7) and day 13 (D13) post-transfection and normalized to levels measured prior to transfection (i.e., at day 0), and the bar on the right corresponds to CFB polynucleotide A to G editing measured at day 13.

FIG. 24 provides a ribbon diagram depicting the structure of complement factor B, where residues corresponding to the indicated protein regions (i.e., oxyanion hole, serine protease active site, salt bridges, cleavage site, verified mutations (i.e., mutations known to be associated with a reduction in CFB activity), or Mg²⁺ binding loop) are shown as spheres. The ribbon diagram of FIG. 24 corresponds to Protein Data Bank accession No. 2ok5, the disclosure of which is incorporated herein by reference in its entirety for all purposes.

FIG. 25 provides a bar graph showing complement factor B (CFB) percent base editing rates measured in HEK293T cells transfected with base editor systems containing an adenosine deaminase base editor (ABE) or a cytidine deaminase base editor (CBE), as indicated, and one of the indicated guide polynucleotides. Base editor systems containing the guide polynucleotide sg23 and a CBE or an ABE were used as positive controls for base editing. The amino acid alterations corresponding to the percent base editing rates represented by each bar are indicated beneath the x-axis. In FIG. 25 the term “Tier 1” refers to the Tier 1 amino acid residues listed in Table 13.

FIG. 26 provides a bar graph showing complement factor B (CFB) percent base editing rates measured in HEK293T cells transfected with base editor systems containing an adenosine deaminase base editor (ABE) or a cytidine deaminase base editor (CBE), as indicated, and one of the indicated guide polynucleotides. Base editor systems containing the guide polynucleotide sg23 and a CBE or an ABE were used as positive controls for base editing. The amino acid alterations corresponding to the percent base editing rates represented by each bar are indicated beneath the x-axis. In FIG. 26 the term “Tier 1” refers to the Tier 1 amino acid residues listed in Table 13.

FIG. 27 provides a bar graph showing complement factor B (CFB) percent base editing rates measured in HEK293T cells transfected with base editor systems containing an adenosine deaminase base editor (ABE) or a cytidine deaminase base editor (CBE), as indicated, and one of the indicated guide polynucleotides. Base editor systems containing the guide polynucleotide sg23 and a CBE or an ABE were used as positive controls for base editing. The amino acid alterations corresponding to the percent base editing rates represented by each bar are indicated beneath the x-axis. In FIG. 27 the term “Tier 2” refers to the Tier 2 amino acid residues listed in Table 13.

FIG. 28 provides a bar graph showing complement factor B (CFB) percent base editing rates measured in HEK293T cells transfected with base editor systems containing an adenosine deaminase base editor (ABE) or a cytidine deaminase base editor (CBE), as indicated, and one of the indicated guide polynucleotides. Base editor systems containing the guide polynucleotide sg23 and a CBE or an ABE were used as positive controls for base editing. The amino acid alterations corresponding to the percent base editing rates represented by each bar are indicated beneath the x-axis. In FIG. 28 the term “Tier 2” refers to the Tier 2 amino acid residues listed in Table 13.

FIG. 29 provides a bar graph showing complement factor B (CFB) percent base editing measured in a primary human hepatocyte (PHH) monolayer transfected with base editor systems containing an adenosine deaminase base editor (ABE) or a cytidine deaminase base editor (CBE), as indicated, and one of the indicated guide polynucleotides. Base editor systems containing the guide polynucleotide sg23, gRNA1193 (TSBTx3826), or gRNA2067 (TSBTx3837) and a CBE or an ABE were used as positive controls for base editing. The amino acid alterations corresponding to the percent base editing rates represented by each bar are indicated beneath the x-axis. The PHH monolayer was administered 200 ng of the indicated guide polynucleotide and 600 ng of mRNA encoding the base editor. FIG. 29 presents a sub-portion of data from FIGS. 25-28.

FIG. 30 provides a schematic diagram showing guide-dependent and guide-independent deamination of a nucleotide of a polynucleotide and lists representative methods by which the same may be predicted or measured. FIG. 30 is adapted from Kempton and Lei, Science, 364:234-236 (2019), the disclosure of which is incorporated herein by reference in its entirety for all purposes.

FIG. 31 provides a bar graph showing an alternative presentation of data from FIG. 23 relating to start codon disruption of CFB in primary human hepatocyte co-cultures. In FIG. 31, each pair of bars represents, from left-to-right, hCFB protein level and A to G editing. In FIG. 31, “dABE (−) Ctrl” indicates negative control base editor systems containing a catalytically inactive base editor. The base editor systems of FIG. 31 (i.e., Sample 1 to Sample 9) are described in Table 12.1D.

FIGS. 32A and 32B provide a bar graph and a schematic diagram relating to a functional assessment of start codon targeting guides in a long-term HepG2 culture system. FIG. 6 provides a bar graph showing base editing rates for the indicated target sites achieved using the indicated active or inactive base editor systems corresponding to Sample 1 to Sample 8, which are described in FIG. 12.1E. FIG. 32B provides a schematic diagram describing the experiment used to collect the data presented in FIG. 32A. In FIG. 32B, “MC” indicates a media change, “TF” indicates transfection with a base editor system, and “NGS” indicates next-generation sequencing.

FIG. 33 provides plots showing human complement factor B (hCFB) protein levels in long-term HepG2 culture systems containing cells transfected with the indicated active (left panel) or inactive (right panel) base editor systems corresponding to Sample 1 to Sample 8, which are described in Table 12.1E, and targeting the indicated sites for editing at the indicated days post-transfection (post-TF). In the left panel of FIG. 33, the lines at 10-days post-TF correspond, from top-to-bottom, to Sample 1, Sample 7, Sample 2, Sample 6/Sample 5, Sample 3, and Sample 4, and the third line from the bottom at 22 days corresponds to Sample 5. In the right panel of FIG. 33, the lines at 10-days post-TF correspond, from top-to-bottom, Sample 1, Sample 6, Sample 2, Sample 3, Sample 7, Sample 4, and Sample 5. “Inactive editors” contained a catalytically inactive base editor.

FIGS. 34A and 34B provide bar graphs showing rates of base editing of complement factor B (CFB) target sites in non-human primates using the indicated base editing systems, which are described in Table 18. FIG. 34A provides a bar graph showing CFB base editing rates observed in two liver biopsies, each taken from a different section of the liver, collected from non-human primates at 15-days post-administration of the indicated base editor systems. FIG. 34B provides a bar graph showing CFB base editing rates observed in the indicated liver sections for the non-human primate at 60-days post-administration of the indicated base editor systems. The doses indicated in FIGS. 34A and 34B are expressed as total gRNA administered. In FIG. 34B, “LLR” represents Liver, left lateral lobe (proximal, distal, and median from the hilus), “LLC” represents Liver, right lateral lobe (proximal and distal from the hilus), “LC” represents Liver, caudate lobe Liver, left median lobe, “LMR” represents Liver, right median lobe, and “LML” represents Liver, center papillary Lung (right diaphragmatic, 2 samples).

FIG. 35 provides plots showing change from baseline of the indicated biomarkers (i.e., AH-50, Bb, and C3a) in non-human primates administered the indicated base editor systems corresponding to Grp1 (Control gRNA) or Grp5 (CFB 1 1.5 mg/kg) of Table 18 at the indicated days following administration of the base editor systems. AH-50 indicates the hemolytic assay measuring alternative pathway. The y-axis of the top panel of FIG. 35 represents percent change from baseline in plasma levels of complement factor B protein (FBL) or Bb. The top panel of FIG. 35 provides a plot showing the mean reductions in plasma full-length Factor B levels (FBL) and the split product of Factor B (Bb) as a percentage of baseline concentration in cynomolgus monkeys that were administered a base editor system intravenously at a dose of 1.5 mg total RNA per kilogram of body weight on day 0. The animals were followed for 57 days. A control group received control LNPs that did not contain base editor systems targeting CFB for editing. The bottom panel of FIG. 35 provides a plot showing the mean reductions in serum alternative complement activity as a percentage of baseline concentration in cynomolgus monkeys that were administered a base editor system intravenously at a dose of 1.5 mg total RNA per kilogram of body weight on day 0. The animals were followed for 57 days. A control group received control LNPs that did not contain base editor systems targeting CFB for editing.

FIG. 36 provides a bar graph showing maximum A to G percent editing at a human complement factor B (hCFB) target site in transgenic mice administered the indicated base editor systems corresponding to Grp1 (ALAS1) and Grp5 (CFB 1 at a total gRNA dose of 0.1 mg/kg (mpk), 0.3 mpk, or 1 mpk) of Table 18. In FIG. 36, “HOM” indicates mice homozygous for hCFB and “HET” indicates mice that are heterozygous for hCFB.

FIG. 37 provides an image of an immunoblot demonstrating that transgenic (Tg) mice heterozygous (Het) for hCFB administered a total gRNA dose of 0.3 mg/kg or 1 mg/kg of the base editor system corresponding to Grp5 of Table 18 showed reduced plasma levels of hCFB at day 15 (D15) post-administration relative to pre-administration (Pre).

FIG. 38 provides a bar graph demonstrating that transgenic (Tg) mice heterozygous (Het) or homozygous (Homo) for hCFB administered the indicated total gRNA dose of the base editor system (“CFB”) corresponding to Grp5 of Table 18 showed reduced plasma levels of hCFB at day 15 (D15) post-administration relative to pre-dosing (PD) and relative to control mice. The term “Control gRNA” in FIG. 38 indicates a base editor system corresponding to Grp1 of Table 18.

FIG. 39 provides a schematic diagram describing the experiment undertaken to collect the data corresponding to FIGS. 40 and 41 and Table 22.

FIG. 40 provides a bar graph showing hCFB base editing rates observed at day 14 post-dosing in the livers of transgenic mice administered the indicated doses of the indicated base editor systems (see also Table 22). The four sets of bars presented in FIG. 40 correspond, from left-to-right, to ALAS1 (one bar; Group 1 of Table 22), CFB 1 NLS (four bars; Groups 2 to 5 of Table 22; Formulation 1), CFB 1 End-Mod (four bars; Groups 6 to 9 of Table 22; Formulation 2), and CFB 2 (four bars; Groups 10 to 13 of Table 22; Formulation 3). Group 3, animal 3012, with a scheduled death on D4 of dosing was excluded from analysis (4.78% editing). Group 4 animals 3013, 3015, and 3016; Group 10 animal 3040; and Group 13 animal 3049 were all found dead and no tissue was preserved for editing assessment.

FIG. 41 provides a bar graph showing results from an Elisa analysis showing percent change from baseline of hCFB protein levels at day 14 post-dosing in the mice of FIG. 40. The terms “NLS”, “End-Mod”, and “Lit Mod1/HMO1” in FIG. 41 correspond to Formulations 1, 2, and 3 of Table 22, respectively.

### DETAILED DESCRIPTION

Provided herein are base editors, endonucleases, and guide RNAs (gRNAs) for use in editing, modifying, or altering a target polynucleotide. In particular embodiments, a base editor or endonuclease of the present disclosure modifies a complement factor B (CFB) polynucleotide. In particular embodiments, a base editor of the invention introduces a stop codon, or missense mutation (e.g., a mutation resulting in a CFB with reduced activity) alteration in a CFB polynucleotide or disrupts a TATA box, start site, or splice site in the CFB polynucleotide. The alterations are associated with a reduction in activity or levels of a CFB polypeptide and/or polynucleotide in a cell.

The invention of the disclosure is based, at least in part, on the discovery that the alternative pathway of the complement system requires the protein factor B for complement pathway amplification and function. The invention is further based, at least in part, upon the discovery that base editing (e.g., disruption of splice acceptor or splice donor, or introduction of a stop codon, missense mutation, or indel alteration) can be used to reduce the expression of a factor B polypeptide in a cell associated with a dysregulated complement system (e.g., inappropriate activation). In particular, reducing activity and/or expression of the factor B polypeptide in a subject diagnosed with a disease or disorder associated with over-activation of the complement system can be an effective treatment strategy. This reduction in activity and/or expression can be effected using any of the base editing systems and/or endonucleases and methods provided herein. Accordingly, the invention features compositions and methods for editing a factor B polynucleotide. The edit to the factor B polynucleotide is associated with a reduction in expression and/or activity of a factor B polypeptide in a cell, tissue, and/or body fluid of a subject, as well as a reduction in symptoms associated with overactivation or otherwise pathogenic activation of the complement system in a subject.

Accordingly, as described in the examples provided herein base editor systems were successfully developed to disrupt complement system activity through functional disruption of factor B at the gene level. Factor B disruption was carried out using two separate approaches: 1) silencing/knock-out of the factor B gene and 2) generation of mutations that disrupted specific factor B functions (see, e.g., those sites listed in Table 14 below).

In embodiments, the methods of the present disclosure include disrupting splicing of a factor B polynucleotide transcript. For example, the base editors or base editor systems provided herein can be used for editing a nucleobase in the splice acceptor situated 5′ of an exon of the factor B polynucleotide. In some embodiments, the target sequence is a splice acceptor in a portion of an intron adjacent to an exon of the factor B polynucleotide and editing a nucleobase in the splice acceptor is associated with a change in the splice acceptor compared to a wild-type splice acceptor site. In some embodiments, the deamination of an A or C nucleobase in the splice acceptor results in disruption of splicing of the mRNA transcript during or after transcription. In some embodiments, the subject has or has the potential to develop a dysregulated and/or over-activated complement system and any disease or disorder associated therewith.

In some instances, the methods of the present disclosure include modifying the factor B polynucleotide to introduce an amino acid alteration in a factor B polypeptide encoded thereby. In embodiments, the amino acid alteration disrupts cleavage of the factor B polypeptide by a plasma factor D to yield a Ba fragment and the active protease Bb fragment.

In some instances, the methods of the present disclosure include modifying a factor B polynucleotide to introduce a stop codon, start site disruption, TATA box disruption, or missense mutation associated with a reduction in levels or activity of the complement factor B polynucleotide and/or polypeptide. The alterations can be effected by a base editor system, such as those described herein.

In some embodiments, the present disclosure provides base editors that efficiently generate an intended mutation, such as a point mutation, in a nucleic acid molecule (e.g., a nucleic acid within a genome of a subject) without generating a significant number of unintended mutations, such as unintended point mutations. In some embodiments, an intended mutation is a mutation that is generated by a base editor system containing a specific base editor (e.g., an adenosine base editor or a cytidine base editor), where the base editor system is specifically designed to generate the intended mutation. In some embodiments, the intended mutation is an adenine (A) to guanine (G) point mutation within the non-coding region of a gene. In some embodiments, the intended mutation is a cytosine (C) to thymine (T) point mutation within the non-coding region of a gene. In some embodiments, the intended mutation is a mutation of a splice acceptor in an intron of a gene associated with a disease or disorder. In some cases, the intended mutation is an indel mutation. In some embodiments, the intended mutation is an adenine (A) to guanine (G) point mutation in the splice acceptor site in an intron of a gene associated with a disease or disorder. In some embodiments, the intended mutation is a missense mutation. The intended mutation can include the introduction of a stop codon to a polynucleotide sequence. In some embodiments, the intended mutation is a mutation that disrupts normal splicing of a complete transcript of a gene, for example, an A to G change in a splice acceptor site within an intron of a disease-causing or a disease-associated gene. In some embodiments, the intended mutation is a mutation in a splice acceptor site that disrupts splicing of a gene transcript and results in an alternative transcript that encodes a truncated and/or nonfunctional protein product.

In some embodiments, any of the base editors or endonucleases provided herein are capable of generating a ratio of intended mutations to unintended mutations (e.g., intended point mutations: unintended point mutations) that is greater than 1:1. In some embodiments, any of the base editors provided herein are capable of generating a ratio of intended mutations to unintended mutations (e.g., intended point mutations: unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more.

In some embodiments, editing of a plurality of nucleobase pairs in one or more genes using the methods provided herein results in formation of at least one intended mutation. In some embodiments, the formation of the at least one intended mutation is in a splice acceptor site and results in disruption of splicing of the mRNA transcript of a disease-associated gene. In some embodiments, the formation of the at least one intended mutation results in a reduction in activity and/or expression of a disease-associated gene. It should be appreciated that multiplex editing can be accomplished using any method or combination of methods provided herein.

The present disclosure provides methods for the treatment of a subject diagnosed with a dysregulated and/or over-activated complement system or any disease or disorder associated therewith. For example, in some embodiments, a method is provided that comprises administering to a subject having or having a propensity to develop a dysregulated and/or over-activated complement system, an effective amount of a nucleobase editor (e.g., an adenosine deaminase base editor or a cytidine deaminase base editor) to effect an alteration in a factor B polynucleotide sequence.

### The Complement System and Factor B

Complement is a system consisting of numerous plasma and cell-bound proteins that plays an important role in both innate and adaptive immunity. The proteins of the complement system act in a series of enzymatic cascades through a variety of protein interactions and cleavage events.

The complement system is a component of the innate immune system and is important for the clearance of pathogens and dead or dying cells. Complement activation results in: formation of a membrane attack complex and cell cytolysis; opsonization of foreign material, targeting it for phagocytosis; and activation of inflammation and diverse immune components. Many complement components are circulating factors primarily produced in the liver.

The complement system plays an important role in defending the body against infectious agents. The complement system contains over 30 serum and cellular proteins that are involved in three major pathways, known as the classical, alternative, and lectin pathways. The classical pathway is typically triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form C3 convertase, which cleaves C3 at a defined cleavage site to form C3a and C3b. Binding of C3b to C3 convertase produces C5 convertase, which cleaves C5 into C5a and C5b. C3a, C4a, and C5a are anaphylotoxins and mediate multiple reactions in the acute inflammatory response. C3a and C5a are also chemotactic factors that attract immune system cells such as neutrophils. Further details relating to C3 are provided in Ricklin, et al. “Complement component C3—The ‘Swiss Army Knife’ of innate immunity and host defense.” Immunol Rev. 2016 November; 274 (1): 33-58; and in Janssen, et al., “Structures of complement component C3 provide insights into the function and evolution of immunity.” Nature. 2005 Sep. 22; 437 (7058): 505-11, the disclosures of which are incorporated herein by reference in their entireties for all purposes.

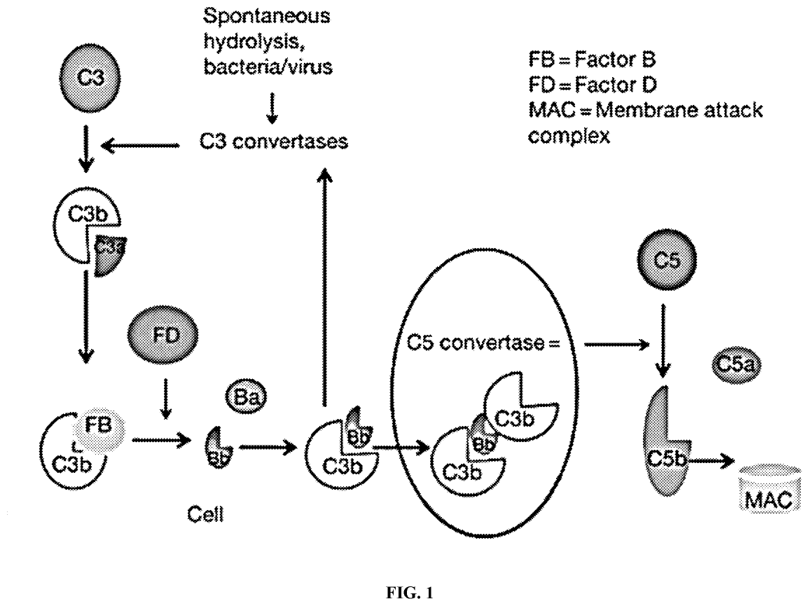

The alternative pathway (see, e.g., FIG. 1) is typically initiated by and amplified at microbial surfaces and various complex polysaccharides. The alternative pathway is triggered by the covalent binding of C3b to a pathogen or cell surface. Next, factor B binds to surface bond C3b, making it susceptible to plasma factor D cleavage. The result is production of Ba and active protease Bb, which remains bound to C3b creating C3bBb, which is the C3 convertase of the alternative complement pathway. This starts the amplification loop with the C3 convertase generating more C3b on the cell surface and the process repeats. Ultimately, there is C3b saturation on the cell surface with release of C3a, a small inflammatory mediator. Eventually, some of the C3b binds to preexisting C3 convertase producing C3b2Bb, which is the alternative pathway's C5 convertase. This cleaves C5 into C5b, which generates the membrane attack complex (MAC), and C5a, a potent proinflammatory mediator. Complement-mediated endothelial cell injury creates a prothrombotic state. It exposes subendothelial collagens and releases vWF and fibrinogen formation. Normally the presence of complement regulatory proteins on cell surfaces prevents significant complement activation from occurring thereon. A more detailed description of the alternative pathway is provided in Keir, L. and Coward, R. J. M., 2011. Pediatr. Nephrol. 26, 523-533, the disclosure of which is incorporated herein by reference in its entirety for all purposes.

Complement factor B (CFB; alternatively “factor B”) is a serine protease and a key component of the complement alternative pathway (AP)/amplification loop. Complement factor D (CFD), another serine protease, cleaves CFB to form Ba and Bb. Bb forms an integral part of the convertase complexes of the AP, which serve to activate the central complement proteins C3 and C5 through proteolytic cleavage.

The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC), also known as the terminal complement complex (TCC). The MAC inserts itself into target cell membranes and causes cell lysis. Small amounts of MAC on the membrane of cells may have a variety of consequences other than cell death. If the TCC does not insert into a membrane, it can circulate in the blood as soluble sC5b-9 (sC5b-9). Levels of sC5b-9 in the blood may serve as an indicator of complement activation.

The lectin complement pathway can be initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase described above.

Accordingly, the present disclosure provides methods for disrupting complement activation by altering a polynucleotide encoding factor B.

Diseases and/or Disorders Associated with Undesirably Increased Activation of the Complement System

Inappropriate activation of the complement system can lead to various diseases and/or disorders in a subject. For example, inappropriate activation of the complement system in a subject damages cells resulting in increased inflammation, the presence of autoantibodies, neural degeneration, and microthrombosis, among others. Inappropriate activation of the complement system is associated with damage to the nervous system (e.g., the Central Nervous System (CNS)), circulatory system, kidneys, eyes, blood cells (e.g., red and white blood cells and platelets), and transplanted organs, as well as damage to other organs or tissues, which may be associated with the presence of micro-emboli. Therefore, an effective treatment for such diseases and/or disorders can involve altering a factor B nucleotide sequence to reduce and/or eliminate expression and/or activity of a factor B polypeptide in a subject, thereby reducing activation of the complement system in an organ, cell, and/or tissue. In embodiments, the organ or tissue is an eye, kidney, nervous system component, heart, or thyroid. Not intending to be bound by theory, complement protein levels in the eye may be dependent on circulating levels of complement proteins generated in the liver.

Some important indications for a subject requiring treatment for inappropriate activation (e.g., overactivation or dysregulation) of the complement system include paroxysmal nocturnal hemoglobinuria (PNH), atypical hemolytic uremic syndrome (aHUS), and IC-MPGN/C3 glomerulopathy. PNH is associated with hemolysis of red blood cells (RBCs) resulting in anemia and thrombosis. The disorder aHUS is associated with hemolysis of RBCs as well as thrombocytopenia and acute kidney failure caused by abnormal clot formation in small blood vessels in the kidney. IC-MPGN and C3 glomerulopathy are associated with kidney malfunction and end-stage renal disease caused by damage to glomeruli of the kidney.

Non-limiting examples of diseases associated inappropriate activation of the complement system include blood disorders, transplant or graft rejection, inflammatory diseases or disorders, eye diseases or disorders, kidney diseases or disorders, heart disorders, respiratory/pulmonary diseases or disorders, autoimmune disorders, inflammatory bowel diseases or disorders, arthritis, neurodegenerative diseases or disorders, musculoskeletal diseases or disorders associated with inflammation, disorders affecting the integumentary system, diseases or disorders affecting the central nervous system, diseases or disorders affecting the circulatory system, diseases or disorders affecting the gastrointestinal system, diseases or disorders affecting the thyroid, chronic pain, allergies, and pulmonary diseases. Further non-limiting examples of diseases associated with inappropriate activation of the complement system include acute antibody-mediated rejection, age-related macular degeneration (e.g. wet or dry age-related macular degeneration), allergic asthma, allergic bronchopulmonary aspergillosis, allergic neuritis, allergic rhinitis, Alzheimer's disease, amyotrophic lateral sclerosis, anaphylaxis, atopic dermatitis, atypical hemolytic syndrome (aHUS), autoimmune diseases, autoimmune hemolytic anemia, Bechet's disease, Behcet's disease, bronchiolitis, bronchiolitis obliterans, C3 glomerulopathy, cancer, central nervous system (CNS) inflammatory disorders, choroidal neovascularization (CNV), choroiditis, chronic allograft vasculopathy, chronic hepatitis, chronic inflammation, chronic inflammatory diseases, chronic muscle inflammation, chronic pain, chronic pancreatitis, chronic rejection of a transplant or graft, chronic urticaria, Churg-Strauss syndrome, conjunctivitis, COVID-19, cyclitis, demyelinating diseases, dermatitis, dermatomyositis, diabetic retinopathy, diseases of the circulatory system, disorders associated with excessive or inappropriate activity of IgE-producing cells, disorders associated with high levels or inappropriate activity of CD4+ helper T cells of the Th17 subtype, encephalitis, eosinophilic pneumonia, eye disorders, geographic atrophy, giant cell arteritis, gingivitis, glaucoma, glomerulonephritis, glomerulonephritis (e.g., membranoproliferative glomerulonephritis or membranous glomerulonephritis), graft rejection or failure, GPA/MPA (granulomatosis with polyangiitis, microscopic), HELLP syndrome, Henoch-Schonlein purpura, hepatitis (e.g. hepatitis C), Huntington's disease, hyperacute rejection, hypersensitivity pneumonitis, idiopathic pulmonary fibrosis (IPF), IgA nephropathy (IgAN), inflammatory bowel diseases (e.g. Crohn's disease or ulcerative colitis), inflammatory joint conditions (e.g. arthritis such as rheumatoid arthritis or psoriatic arthritis, juvenile chronic arthritis, spondyloarthropathies Reiter's syndrome, or gout), inflammatory skin diseases, infusion reaction, interstitial pneumonia, iridocyclitis, iritis, ischemia/reperfusion injury, Kawasaki disease, keratitis, lupus nephritis, membranoproliferative glomerulonephritis (MPGN) (e.g. MPGN type I, type II, or type III), meningitis, microscopic polyangiitis, multiple sclerosis (MS), myasthenia gravis, myocarditis, nasal polyposis, neurodegenerative diseases, neuromyelitis optica, neuromyelitis optica (NMO), neuropathic pain, ocular inflammation, osteoarthritis, pancreatitis, panniculitis, Parkinson's disease, paroxysmal nocturnal hemoglobinuria (PNH), pars planitis, pathologic immune responses to tissue/organ transplantation, pemphigoid, pemphigus, periodontitis, persistent asthma, polyarteritis nodosa, polymyositis, primary membranous nephropathy, proliferative vitreoretinopathy, proteinuria, psoriasis, pulmonary fibrosis (e.g. idiopathic pulmonary fibrosis), radiation-induced lung injury, renal disease, respiratory disease or disorders (e.g. asthma or chronic obstructive pulmonary disease (COPD), oridiopathic pulmonary fibrosis, or asthma), respiratory distress syndrome, retinal neovascularization (RNV), retinopathy of prematurity, rheumatoid arthritis (RA), rhinosinusitis, sarcoid, sarcoidosis, scleritis, scleroderma, sclerodermatomyositis, sclerosis, sepsis, Sjögren syndrome, Sjoren's syndrome, stroke, systemic lupus erythematosus, systemic scleroderma, Takayasu's arteritis, Th2-associated disorders (e.g. a disorder associated with high levels or high activation of CD4+ helper T cells of the Th2 subtype), thyroiditis (e.g. Hashimoto's thyroiditis, Graves' disease, or post-partum thyroiditis), thyroiditis, transplant damage, transplant rejection, ulcerative colitis, uveitis, vasculitis, and Wegener's granulomatosis. In embodiments, the methods of the invention involve reducing complement-mediated hemolysis in a subject. Further non-limiting examples of diseases include Creutzfeldt-Jakob disease, Pick's disease, mild cognitive impairment, fibromyalgia, frontotemporal dementia, dementia with Lewy bodies, multiple system atrophy, chronic inflammatory, demyelinating polyneuropathy, Guillain-Barré syndrome, multifocal motor neuropathy, non-alcoholic fatty liver disease (NAFLD) e.g., non-alcoholic steatohepatitis (NASH), and Stargardt macular dystrophy. Paroxysmal nocturnal hemoglobinuria is associated with mutations in PigA (Phosphatidyl inositol glycan anchor biosynthesis class a) that prevent GPI-anchor production and attachment of CD59 and CD55 to red blood cells (RBCs), which leads to the lysis of RBCs.

In some embodiments, inappropriate activation of the complement system is implicated in the progression and pathogenesis of a disease selected from glaucoma, diabetic retinopathy, age-related macular degeneration, and neurological diseases such as amyotrophic lateral sclerosis (ALS), multiple sclerosis (MS), Alzheimer's disease, and various Tauopathies.

Existing treatments for diseases associated with inappropriate activation of the complement system often require regular, sometimes invasive, dosing regimens. There is, therefore, a present need for improved treatments for diseases associated with inappropriate activation of the complement system.

The methods and compositions of the present disclosure are suitable in embodiments for use in treatment of any of the above-listed diseases or disorders related to improper activation of the complement system. In various instances, the methods involve introducing a modification to a factor B polynucleotide that results in reduced expression and/or activity of a factor B polypeptide in a cell.

### Editing of Target Genes

Exemplary spacer sequences and guide polynucleotide sequences suitable for use in guide RNAs that can be used to produce the polynucleotide edits described herein (e.g., missense mutations, introduction of stop codons, splice-site disruption mutations, TATA box alterations, start codon alterations, etc.) are listed in Tables 1A to 2H below. To produce the polynucleotide edits, cells (e.g., cells in or from a subject) are contacted with one or more guide RNAs containing one or more of the spacer sequences listed in Tables 2A to 2H below, or fragments thereof, and a nucleobase editor polypeptide or complex containing a nucleic acid programmable DNA binding protein (napDNAbp) and one or more deaminases with cytidine deaminase and/or adenosine deaminase activity (e.g., a “dual deaminase” which has cytidine and adenosine deaminase activity). In embodiments, the base editor and/or endonuclease is introduced to the cell using a polynucleotide sequence (e.g., mRNA) encoding the base editor and/or endonuclease. Tables 1A to 1H below list representative guide polynucleotide sequences suitable for use in methods of the disclosure for altering a CFB polynucleotide. Tables 2A to 2H below list representative guide RNA spacer sequences that may be used in various embodiments in combination with indicated base editors. In embodiments, guide RNAs containing the spacer sequences listed in Tables 2A to 2H may be used to target the target sequences listed in Tables 2A to 2H, optionally to effect the edits (e.g., amino acid or nucleotide alterations) listed in any of Tables 2A to 2H. In some instances, the gRNA is added directly to a cell. In some embodiments, the gRNA comprises nucleotide analogs. These nucleotide analogs can inhibit degradation of the gRNA from cellular processes. Tables 2A to 2H provide target sequences to be used for gRNAs. Further exemplary spacer sequences suitable for use in gRNA sequences for use in the methods provided herein include fragments of any of the spacers provided in Tables 2A to 2H as well as any of the spacers provided in Tables 2A to 2H modified to include an extension or truncation at the 3′ and/or 5′ end(s). In embodiments, a spacer sequence of Tables 2A to 2H can be modified to include a 1, 2, 3, 4, 5, 6, 7, 8, 9, or 10 nucleotide extension or truncation at the 3′ and/or 5′ end(s).

Variants of the spacer sequences provided herein comprising 1, 2, 3, 4, or 5 nucleobase alterations are contemplated. For example, variation of a target polynucleotide sequence within a population (e.g., single nucleotide polymorphisms) may require said alterations to a spacer sequence to allow the spacer to better bind a variant of a target sequence in a subject.

In various instances, it is advantageous for a spacer sequence to include a 5′ and/or a 3′ “G” nucleotide. In some cases, for example, any spacer sequence or guide polynucleotide provided herein comprises or further comprises a 5′ “G”, where, in some embodiments, the 5′ “G” is or is not complementary to a target sequence. In some embodiments, the 5′ “G” is added to a spacer sequence that does not already contain a 5′ “G.” For example, it can be advantageous for a guide RNA to include a 5′ terminal “G” when the guide RNA is expressed under the control of a U6 promoter or the like because the U6 promoter prefers a “G” at the transcription start site (see Cong, L. et al. “Multiplex genome engineering using CRISPR/Cas systems. Science 339:819-823 (2013) doi: 10.1126/science.1231143). In some cases, a 5′ terminal “G” is added to a guide polynucleotide that is to be expressed under the control of a promoter but is optionally not added to the guide polynucleotide if or when the guide polynucleotide is not expressed under the control of a promoter.

In some embodiments, a guide polynucleotide of the disclosure contains a spacer and scaffold containing one of the following nucleotide modification schemes (“mod schemes”), where “N” represents any nucleotide, “mN” indicates a 2′-OMe modification of the nucleotide “N”, and “Ns” indicates that the nucleotide “N” is linked to the following nucleotide by a phosphorothioate (PS):

End-mod SpCas9 guide polynucleotide
(SEQ ID NO: 440)
mNsmNsmNsNNNNNNNNNNNNNNNNNGUUUUAGAGCUAGAAAUAGCAAGU
UAAAAUAAGGCUAGUCCGUUAUCAACUUGAAAAAGUGGCACCGAGUCGGU
GCUsmUsmUsmU
End-mod SaCas9 guide polynucleotide
(SEQ ID NO: 441)
mNsmNsmNsNNNNNNNNNNNNNNNNNNGUUUUAGUACUCUGUAAUGAAAA
UUACAGAAUCUACUAAAACAAGGCAAAAUGCCGUGUUUAUCUCGUCAACU
UGUUGGCGAGAUsmUsmUsmU
HM01:
(SEQ ID NO: 440)
mNsmNsmNsNNNNNNNNNNNNNNNNNGUUUUAGAmGmCmUmAmGmAmAmA
mUmAmGmCAAGUUAAAAUAAGGCUAGUCCGUUAUCAmAmCmUmUmGmAmA
mAmAmAmGmUmGmGmCmAmCmCmGmAmGmUmCmGmGmUmGmCmUsmUsmU
smU
HM07:
(SEQ ID NO: 440)
mNsmNsmNsmNmNmNmNmNmNmNNNNNNNNNNNmGUUUUAGmAmGmCmUm
AmGmAmAmAmUmAmGmCmAmAGUUmAAmAAmUAmAmGmGmCmUmAGUmCm
CGUUAmUmCAAmCmUmUmGmAmAmAmAmAmGmUmGGmCmAmCmCmGmAmG
mUmCmGmGmUmGmCmUsmUsmUsmU
NLS (bpsv40):
(SEQ ID NOs: 440 and 446)
mNsmNsmNsNNNNNNNNNNNNNNNNNGUUUUAGAGCUAGAAAUAGCAAGU
UAAAAUAAGGCUAGUCCGUUAUCAACUUGAAAAAGUGGCACCGAGUCGGU
GCmUsmUsmUsmU-NHC6-CrossL-ac-CKRTADGSEFESPKKKRKV
LONGEST:
(SEQ ID NO: 444)
mNsmNsmNsNNNNNNNNNNNNNNNNNGUUUUAGAmGmCmCmGmGmCmGmG
mAmAmAmCmGmCmCmGmGmCAAGUUAAAAUAAGGCUAGUCCGUUAUCAmA
mCmUmUmGmAmAmAmAmAmGUGmGmCmAmCmCmGmAmGmUmCmGmGmUmG
mCmUsmUsmUsmU
NLS + LONGEST:
(SEQ ID NOs: 445 and 446)
mNsmNsmNsNNNNNNNNNNNNNNNNNGUUUUAGAmGmCmCmGmGmCmGmG
mAmAmAmCmGmCmCmGmGmCAAGUUAAAAUAAGGCUAGUCCGUUAUCAmA
mCmUmUmGmAmAmAmAmAmGmUmGmGmCmAmCmCmGmAmGmUmCmGmGmU
mGmCmUsmUsmUsmU-NHC5-CrossL-CKRTADGSEFESPKKKRKV
LONGEST + GOLD:
(SEQ ID NO: 447)
mNsmNsmNsNNNNNNNNNNNNNNNNNGUUUUAGAmGmCmCmGmGmCmGmG
mAmAmAmCmGmCmCmGmGmCAAGUUAAAAUAAGGCUAGUCCGUUAmUmCA
AmCmUmUGGACUUCGGUCCmAmAmGUGGmCmAmCmCmGmAmGmUmCmGmG
mUmGmCmUsmUsmUsmU

In embodiments, any of the above guide sequences, the number of N nucleotides (i.e., the spacer sequence) can vary between 15 and 25. In some cases, the number of N nucleotides is 18, 19, 20, 21, 22, or 23.

Exemplary guide RNA sequences are provided in the following Tables 1A-1I and 2A-2H. Throughout the tables, the ranges (e.g., 3-9) in the guide polynucleotide names indicate the base editing window for an exemplary base editor suitable for use with the guide polynucleotide (e.g., nucleotides 3 to 9, where location 1 is the first nucleobase complementary to the spacer and adjacent to the protospacer adjacent motif).

TABLE 1A
Representative sequences for guide polynucleotides for
use in guiding a base editor to alter a complement
factor B splice site.¹

| Guide | | |
|---|---|---|
| poly- | | SEQ |
| nucleotide | | ID |
| name | Guide polynucleotide sequence | NO |
| gRNA2055 | mCsmUsmCsAGAUGUCUAUGUGUUUGUUUUAGAGCUAGAAAUAGCAAGU | 448 |
| | UAAAAUAAGGCUAGUCCGUUAUCAACUUGAAAAAGUGGCACCGAGUCGG | |
| | UGCUsmUsmUsmU | |
| gRNA1193 | mCsmCsmUsCAGAUGUCUAUGUGUUUGUUUUAGAGCUAGAAAUAGCAAG | 449 |
| | UUAAAAUAAGGCUAGUCCGUUAUCAACUUGAAAAAGUGGCACCGAGUCG | |
| | GUGCUsmUsmUsmU | |
| gRNA2057 | mCsmCsmCsUCAGAUGUCUAUGUGUUUGUUUUAGAGCUAGAAAUAGCAA | 450 |
| | GUUAAAAUAAGGCUAGUCCGUUAUCAACUUGAAAAAGUGGCACCGAGUC | |
| | GGUGCUsmUsmUsmU | |
| gRNA2058 | mUsmCsmCsCUCAGAUGUCUAUGUGUUUGUUUUAGAGCUAGAAAUAGCA | 451 |
| | AGUUAAAAUAAGGCUAGUCCGUUAUCAACUUGAAAAAGUGGCACCGAGU | |
| | CGGUGCUsmUsmUsmU | |
| gRNA2059 | mAsmUsmCsCCUCAGAUGUCUAUGUGUUUGUUUUAGAGCUAGAAAUAGC | 452 |
| | AAGUUAAAAUAAGGCUAGUCCGUUAUCAACUUGAAAAAGUGGCACCGAG | |
| | UCGGUGCUsmUsmUsmU | |
| gRNA2060 | mCsmUsmCsACCUCUGCAAGUAUUGGUUUUAGAGCUAGAAAUAGCAAGU | 453 |
| | UAAAAUAAGGCUAGUCCGUUAUCAACUUGAAAAAGUGGCACCGAGUCGG | |
| | UGCUsmUsmUsmU | |
| gRNA1202 | mUsmCsmUsCACCUCUGCAAGUAUUGGUUUUAGAGCUAGAAAUAGCAAG | 454 |
| | UUAAAAUAAGGCUAGUCCGUUAUCAACUUGAAAAAGUGGCACCGAGUCG | |
| | GUGCUsmUsmUsmU | |
| gRNA2062 | mCsmUsmCsUCACCUCUGCAAGUAUUGGUUUUAGAGCUAGAAAUAGCAA | 455 |
| | GUUAAAAUAAGGCUAGUCCGUUAUCAACUUGAAAAAGUGGCACCGAGUC | |
| | GGUGCUsmUsmUsmU | |
| gRNA2063 | mUsmCsmUsCUCACCUCUGCAAGUAUUGGUUUUAGAGCUAGAAAUAGCA | 456 |
| | AGUUAAAAUAAGGCUAGUCCGUUAUCAACUUGAAAAAGUGGCACCGAGU | |
| | CGGUGCUsmUsmUsmU | |
| gRNA2064 | mUsmUsmCsUCUCACCUCUGCAAGUAUUGGUUUUAGAGCUAGAAAUAGC | 457 |
| | AAGUUAAAAUAAGGCUAGUCCGUUAUCAACUUGAAAAAGUGGCACCGAG | |
| | UCGGUGCUsmUsmUsmU | |
| gRNA2065 | mCsmUsmUsACAAUGACUGAGAUCUGUUUUAGAGCUAGAAAUAGCAAGU | 458 |
| | UAAAAUAAGGCUAGUCCGUUAUCAACUUGAAAAAGUGGCACCGAGUCGG | |
| | UGCUsmUsmUsmU | |
| gRNA1204 | mGsmCsmUSUACAAUGACUGAGAUCUGUUUUAGAGCUAGAAAUAGCAAG | 459 |
| | UUAAAAUAAGGCUAGUCCGUUAUCAACUUGAAAAAGUGGCACCGAGUCG | |
| | GUGCUsmUsmUsmU | |
| gRNA2067 | mUsmGsmCsUUACAAUGACUGAGAUCUGUUUUAGAGCUAGAAAUAGCAA | 460 |
| | GUUAAAAUAAGGCUAGUCCGUUAUCAACUUGAAAAAGUGGCACCGAGUC | |
| | GGUGCUsmUsmUsmU | |
| gRNA2068 | mGsmUsmGsCUUACAAUGACUGAGAUCUGUUUUAGAGCUAGAAAUAGCA | 461 |
| | AGUUAAAAUAAGGCUAGUCCGUUAUCAACUUGAAAAAGUGGCACCGAGU | |
| | CGGUGCUsmUsmUsmU | |
| gRNA2069 | mUsmGsmUsGCUUACAAUGACUGAGAUCUGUUUUAGAGCUAGAAAUAGC | 462 |
| | AAGUUAAAAUAAGGCUAGUCCGUUAUCAACUUGAAAAAGUGGCACCGAG | |
| | UCGGUGCUsmUsmUsmU | |
| gRNA2070 | mCsmUsmUsACAGUGACUGAGAUCUGUUUUAGAGCUAGAAAUAGCAAGU | 463 |
| | UAAAAUAAGGCUAGUCCGUUAUCAACUUGAAAAAGUGGCACCGAGUCGG | |
| | UGCUsmUsmUsmU | |
| gRNA1999 | mGsmCsmUsUACAGUGACUGAGAUCUGUUUUAGAGCUAGAAAUAGCAAG | 464 |
| | UUAAAAUAAGGCUAGUCCGUUAUCAACUUGAAAAAGUGGCACCGAGUCG | |
| | GUGCUsmUsmUsmU | |
| gRNA2072 | mUsmGsmCsUUACAGUGACUGAGAUCUGUUUUAGAGCUAGAAAUAGCAA | 465 |
| | GUUAAAAUAAGGCUAGUCCGUUAUCAACUUGAAAAAGUGGCACCGAGUC | |
| | GGUGCUsmUsmUsmU | |
| gRNA2073 | mGsmUsmGsCUUACAGUGACUGAGAUCUGUUUUAGAGCUAGAAAUAGCA | 466 |
| | AGUUAAAAUAAGGCUAGUCCGUUAUCAACUUGAAAAAGUGGCACCGAGU | |
| | CGGUGCUsmUsmUsmU | |
| gRNA2074 | mUsmGsmUsGCUUACAGUGACUGAGAUCUGUUUUAGAGCUAGAAAUAGC | 467 |
| | AAGUUAAAAUAAGGCUAGUCCGUUAUCAACUUGAAAAAGUGGCACCGAG | |
| | UCGGUGCUsmUsmUsmU | |

¹“mN” indicates a 2′-OMe modification of the nucleotide “N”, and “Ns” indicates that the nucleotide “N” is linked to the following (i.e., 3′) nucleotide by a phosphorothioate (PS).

TABLE 1B
Representative sequences for guide polynucleotides for
use in guiding a base editor to introduce a missense
mutation to a complement factor B polynucleotide.²

| Guide | | SEQ |
|---|---|---|
| polynucleotide | | ID |
| name | Guide polynucleotide sequence | NO |
| GuideDesign14_ABE_ | mCsmAsmGsAAUCACCUGCAAGGAGAGUUUUAGAGCUAGAAAUA | 468 |
| NGA_20nt_3-9_005_- | GCAAGUUAAAAUAAGGCUAGUCCGUUAUCAACUUGAAAAAGUGG | |
| _31951540_spCas9 | CACCGAGUCGGUGCUsmUsmUsmU | |
| GuideDesign14_ABE_ | mCsmCsmAsGAAUCACCUGCAAGGAGGUUUUAGAGCUAGAAAUA | 469 |
| NGG_20nt_3-9_002_- | GCAAGUUAAAAUAAGGCUAGUCCGUUAUCAACUUGAAAAAGUGG | |
| _31951541_spCas9 | CACCGAGUCGGUGCUsmUsmUsmU | |
| GuideDesign14_ABE_ | mGsmCsmCsCACCUUCCUUGUGCCAAUGUUUUAGUACUCUGUAA | 470 |
| NNNRRT_21nt_5- | UGAAAAUUACAGAAUCUACUAAAACAAGGCAAAAUGCCGUGUUU | |
| 14_014_- | AUCUCGUCAACUUGUUGGCGAGAUsmUsmUsmU | |
| _31947370_saCas9 | | |
| GuideDesign14_ABE_ | mCsmUsmGsGCUGCCCACCUUCCUUGUGUUUUAGUACUCUGUAA | 471 |
| NNNRRT_21nt_5- | UGAAAAUUACAGAAUCUACUAAAACAAGGCAAAAUGCCGUGUUU | |
| 14_014_- | AUCUCGUCAACUUGUUGGCGAGAUsmUsmUsmU | |
| _31947376_saCas9 | | |
| GuideDesign14_ABE_ | mUsmGsmCsUGUCUGAUCCAUCUAGCAGUUUUAGUACUCUGUAA | 472 |
| NNNRRT_21nt_5- | UGAAAAUUACAGAAUCUACUAAAACAAGGCAAAAUGCCGUGUUU | |
| 14_014_- | AUCUCGUCAACUUGUUGGCGAGAUsmUsmUsmU | |
| _31947998_saCas9 | | |
| GuideDesign14_ABE_ | mAsmCsmAsGCAGCACAUUGUUUCACUGUUUUAGUACUCUGUAA | 473 |
| NNNRRT_21nt_5- | UGAAAAUUACAGAAUCUACUAAAACAAGGCAAAAUGCCGUGUUU | |
| 14_014_+_31950345 | AUCUCGUCAACUUGUUGGCGAGAUsmUsmUsmU | |
| saCas9 | | |
| GuideDesign14_CBE_ | mGsmCsmAsGCACAUUGUUUCACUGUGUUUUAGAGCUAGAAAUA | 474 |
| NGA_20nt_4- | GCAAGUUAAAAUAAGGCUAGUCCGUUAUCAACUUGAAAAAGUGG | |
| 9_006_+_31950348_ | CACCGAGUCGGUGCUsmUsmUsmU | |
| spCas9 | | |
| GuideDesign14_CBE_ | mGsmCsmAsCAUUGUUUCACUGUGGAGUUUUAGAGCUAGAAAUA | 475 |
| NGA_20nt_4- | GCAAGUUAAAAUAAGGCUAGUCCGUUAUCAACUUGAAAAAGUGG | |
| 9_006_+_31950351_ | CACCGAGUCGGUGCUsmUsmUsmU | |
| spCas9 | | |
| GuideDesign14_CBE_ | mAsmCsmGsUUCGCCGCUGGGAGCCAGUUUUAGAGCUAGAAAUA | 476 |
| NGC_20nt_4-9_009_- | GCAAGUUAAAAUAAGGCUAGUCCGUUAUCAACUUGAAAAAGUGG | |
| _31947453_spCas9 | CACCGAGUCGGUGCUsmUsmUsmU | |
| GuideDesign14_CBE_ | mCsmAsmAsUGCUGUCUGAUCCAUCUGUUUUAGAGCUAGAAAUA | 477 |
| NGC_20nt_4-9_009_- | GCAAGUUAAAAUAAGGCUAGUCCGUUAUCAACUUGAAAAAGUGG | |
| _31948005_spCas9 | CACCGAGUCGGUGCUsmUsmUsmU | |
| GuideDesign14_CBE_ | mGsmAsmUsGGAUCAGACAGCAUUGGGUUUUAGAGCUAGAAAUA | 478 |
| NGC_20nt_4- | GCAAGUUAAAAUAAGGCUAGUCCGUUAUCAACUUGAAAAAGUGG | |
| 9_009_+_31948009_ | CACCGAGUCGGUGCUsmUsmUsmU | |
| spCas9 | | |
| GuideDesign14_CBE_ | mGsmAsmUsCAGACAGCAUUGGGGCCGUUUUAGAGCUAGAAAUA | 479 |
| NGC_20nt_4- | GCAAGUUAAAAUAAGGCUAGUCCGUUAUCAACUUGAAAAAGUGG | |
| 9_009_+_31948013_ | CACCGAGUCGGUGCUsmUsmUsmU | |
| spCas9 | | |
| GuideDesign14_CBE_ | mUsmCsmAsGGGACUAACACCAAGAAGUUUUAGAGCUAGAAAUA | 480 |
| NGC_20nt_4- | GCAAGUUAAAAUAAGGCUAGUCCGUUAUCAACUUGAAAAAGUGG | |
| 9_009_+_31948843_ | CACCGAGUCGGUGCUsmUsmUsmU | |
| spCas9 | | |
| GuideDesign14_CBE_ | mCsmCsmAsCCUUCCUUGUGCCAAUGGUUUUAGAGCUAGAAAUA | 481 |
| NGG_20nt_4-9_003_ | GCAAGUUAAAAUAAGGCUAGUCCGUUAUCAACUUGAAAAAGUGG | |
| -_31947372_spCas9 | CACCGAGUCGGUGCUsmUsmUsmU | |
| GuideDesign14_CBE_ | mCsmCsmCsACCUUCCUUGUGCCAAUGUUUUAGAGCUAGAAAUA | 482 |
| NGG_20nt_4-9_003_ | GCAAGUUAAAAUAAGGCUAGUCCGUUAUCAACUUGAAAAAGUGG | |
| -_31947373_spCas9 | CACCGAGUCGGUGCUsmUsmUsmU | |
| GuideDesign14_CBE_ | mGsmCsmCsCACCUUCCUUGUGCCAAGUUUUAGAGCUAGAAAUA | 483 |
| NGG_20nt_4-9_003_ | GCAAGUUAAAAUAAGGCUAGUCCGUUAUCAACUUGAAAAAGUGG | |
| -_31947374_spCas9 | CACCGAGUCGGUGCUsmUsmUsmU | |
| GuideDesign14_CBE_ | mGsmUsmCsAGGGACUAACACCAAGAGUUUUAGAGCUAGAAAUA | 484 |
| NGG_20nt_4- | GCAAGUUAAAAUAAGGCUAGUCCGUUAUCAACUUGAAAAAGUGG | |
| 9_003_+_31948842_ | CACCGAGUCGGUGCUsmUsmUsmU | |
| spCas9 | | |
| GuideDesign14_CBE_ | mAsmGsmCsAGCACAUUGUUUCACUGGUUUUAGAGCUAGAAAUA | 485 |
| NGG_20nt_4- | GCAAGUUAAAAUAAGGCUAGUCCGUUAUCAACUUGAAAAAGUGG | |
| 9_003_+_31950347_ | CACCGAGUCGGUGCUsmUsmUsmU | |
| spCas9 | | |
| GuideDesign14_CBE_ | mGsmCsmCsCACCUUCCUUGUGCCAAUGUUUUAGUACUCUGUAA | 486 |
| NNGRRT_21nt_3- | UGAAAAUUACAGAAUCUACUAAAACAAGGCAAAAUGCCGUGUUU | |
| 12_012_- | AUCUCGUCAACUUGUUGGCGAGAUsmUsmUsmU | |
| _31947370_saCas9 | | |
| GuideDesign14_CBE_ | mAsmCsmAsGCAGCACAUUGUUUCACUGUUUUAGUACUCUGUAA | 487 |
| NNGRRT_21nt_3- | UGAAAAUUACAGAAUCUACUAAAACAAGGCAAAAUGCCGUGUUU | |
| 12_012_+_31950345_ | AUCUCGUCAACUUGUUGGCGAGAUsmUsmUsmU | |
| saCas9 | | |
| GuideDesign14_CBE_ | mGsmCsmCsCACCUUCCUUGUGCCAAUGUUUUAGUACUCUGUAA | 488 |
| NNNRRT_21nt_3- | UGAAAAUUACAGAAUCUACUAAAACAAGGCAAAAUGCCGUGUUU | |
| 12_015_- | AUCUCGUCAACUUGUUGGCGAGAUsmUsmUsmU | |
| _31947370_saCas9 | | |
| GuideDesign14_CBE_ | mCsmUsmGsGCUGCCCACCUUCCUUGUGUUUUAGUACUCUGUAA | 489 |
| NNNRRT_21nt_3- | UGAAAAUUACAGAAUCUACUAAAACAAGGCAAAAUGCCGUGUUU | |
| 12_015_- | AUCUCGUCAACUUGUUGGCGAGAUsmUsmUsmU | |
| _31947376_saCas9 | | |
| GuideDesign14_CBE_ | mCsmGsmUsUCGCCGCUGGGAGCCACGGUUUUAGUACUCUGUAA | 490 |
| NNNRRT_21nt_3- | UGAAAAUUACAGAAUCUACUAAAACAAGGCAAAAUGCCGUGUUU | |
| 12_015_- | AUCUCGUCAACUUGUUGGCGAGAUsmUsmUsmU | |
| _31947448_saCas9 | | |
| GuideDesign14_CBE_ | mUsmCsmUsUCCGCUUCUGUUGUUCCCGUUUUAGUACUCUGUAA | 491 |
| NNNRRT_21nt_3- | UGAAAAUUACAGAAUCUACUAAAACAAGGCAAAAUGCCGUGUUU | |
| 12_015_- | AUCUCGUCAACUUGUUGGCGAGAUsmUsmUsmU | |
| _31947938_saCas9 | | |
| GuideDesign14_CBE_ | mUsmGsmCsUGUCUGAUCCAUCUAGCAGUUUUAGUACUCUGUAA | 492 |
| NNNRRT_21nt_3- | UGAAAAUUACAGAAUCUACUAAAACAAGGCAAAAUGCCGUGUUU | |
| 12_015_- | AUCUCGUCAACUUGUUGGCGAGAUsmUsmUsmU | |
| _31947998_saCas9 | | |
| GuideDesign14_CBE_ | mAsmAsmCsGUCAUAGUCAUAAAAUUCGUUUUAGUACUCUGUAA | 493 |
| NNNRRT_21nt_3- | UGAAAAUUACAGAAUCUACUAAAACAAGGCAAAAUGCCGUGUUU | |
| 12_015_- | AUCUCGUCAACUUGUUGGCGAGAUsmUsmUsmU | |
| _31950698_saCas9 | | |
| GuideDesign14_CBE_ | mAsmCsmAsGCAGCACAUUGUUUCACUGUUUUAGUACUCUGUAA | 494 |
| NNNRRT_21nt_3- | UGAAAAUUACAGAAUCUACUAAAACAAGGCAAAAUGCCGUGUUU | |
| 12_015_+_31950345 | AUCUCGUCAACUUGUUGGCGAGAUsmUsmUsmU | |
| saCas9 | | |
| GuideDesign14_CBE_ | mUsmGsmCsAGGUGAUUCUGGCGGCCCGUUUUAGUACUCUGUAA | 495 |
| NNNRRT_21nt_3- | UGAAAAUUACAGAAUCUACUAAAACAAGGCAAAAUGCCGUGUUU | |
| 12_015_+_31951549 | AUCUCGUCAACUUGUUGGCGAGAUsmUsmUsmU | |
| saCas9 | | |
| GuideDesign15_ABE_ | mCsmCsmCsAGGGGAACAACAGAAGCGUUUUAGAGCUAGAAAUA | 496 |
| NGA_20nt_3- | GCAAGUUAAAAUAAGGCUAGUCCGUUAUCAACUUGAAAAAGUGG | |
| 9_005_+_31947939_ | CACCGAGUCGGUGCUsmUsmUsmU | |
| spCas9 | | |
| GuideDesign15_ABE_ | mAsmGsmGsGGAACAACAGAAGCGGAGUUUUAGAGCUAGAAAUA | 497 |
| NGA_20nt_3- | GCAAGUUAAAAUAAGGCUAGUCCGUUAUCAACUUGAAAAAGUGG | |
| 9_005_+_31947942_ | CACCGAGUCGGUGCUsmUsmUsmU | |
| spCas9 | | |
| GuideDesign15_ABE_ | mCsmAsmGsAAGCGGAAGAUCGUCCUGUUUUAGAGCUAGAAAUA | 498 |
| NGA_20nt_3- | GCAAGUUAAAAUAAGGCUAGUCCGUUAUCAACUUGAAAAAGUGG | |
| 9_005_+_31947952_ | CACCGAGUCGGUGCUsmUsmUsmU | |
| spCas9 | | |
| GuideDesign15_ABE_ | mGsmCsmCsAGAUGAAAGCCAGUCUCGUUUUAGAGCUAGAAAUA | 499 |
| NGA_20nt_3- | GCAAGUUAAAAUAAGGCUAGUCCGUUAUCAACUUGAAAAAGUGG | |
| 9_005_+_31950044_ | CACCGAGUCGGUGCUsmUsmUsmU | |
| spCas9 | | |
| GuideDesign15_ABE_ | mAsmCsmAsGAAGCGGAAGAUCGUCCGUUUUAGAGCUAGAAAUA | 500 |
| NGG_20nt_3- | GCAAGUUAAAAUAAGGCUAGUCCGUUAUCAACUUGAAAAAGUGG | |
| 9_002_+_31947951_ | CACCGAGUCGGUGCUsmUsmUsmU | |
| spCas9 | | |
| GuideDesign15_ABE_ | mAsmGsmAsUGGAUCAGACAGCAUUGGUUUUAGAGCUAGAAAUA | 501 |
| NGG_20nt_3- | GCAAGUUAAAAUAAGGCUAGUCCGUUAUCAACUUGAAAAAGUGG | |
| 9_002_+_31948008_ | CACCGAGUCGGUGCUsmUsmUsmU | |
| spCas9 | | |
| GuideDesign15_ABE_ | mCsmUsmGsCCAGAUGAAAGCCAGUCUGUUUUAGUACUCUGUAA | 502 |
| NNGRRT_21nt_5- | UGAAAAUUACAGAAUCUACUAAAACAAGGCAAAAUGCCGUGUUU | |
| 14_011_+_31950042 | AUCUCGUCAACUUGUUGGCGAGAUsmUsmUsmU | |
| saCas9 | | |
| GuideDesign15_ABE_ | mCsmCsmCsAGGGGAACAACAGAAGCGGUUUUAGUACUCUGUAA | 503 |
| NNNRRT_21nt_5- | UGAAAAUUACAGAAUCUACUAAAACAAGGCAAAAUGCCGUGUUU | |
| 14_014_+_31947939_ | AUCUCGUCAACUUGUUGGCGAGAUsmUsmUsmU | |
| saCas9 | | |
| GuideDesign15_ABE_ | mCsmUsmGsCCAGAUGAAAGCCAGUCUGUUUUAGUACUCUGUAA | 504 |
| NNNRRT_21nt_5- | UGAAAAUUACAGAAUCUACUAAAACAAGGCAAAAUGCCGUGUUU | |
| 14_014_+_31950042_ | AUCUCGUCAACUUGUUGGCGAGAUsmUsmUsmU | |
| saCas9 | | |
| GuideDesign15_CBE_ | mUsmUsmGsUUCCCCUGGGUGCCAGGGUUUUAGAGCUAGAAAUA | 505 |
| NGA_20nt_4-9_006_ | GCAAGUUAAAAUAAGGCUAGUCCGUUAUCAACUUGAAAAAGUGG | |
| -_31947929_spCas9 | CACCGAGUCGGUGCUsmUsmUsmU | |
| GuideDesign15_CBE_ | mUsmGsmAsUCCAUCUAGCACCAGGUGUUUUAGAGCUAGAAAUA | 506 |
| NGA_20nt_4-9_006_ | GCAAGUUAAAAUAAGGCUAGUCCGUUAUCAACUUGAAAAAGUGG | |
| -_31947995_spCas9 | CACCGAGUCGGUGCUsmUsmUsmU | |
| GuideDesign15_CBE_ | mAsmCsmCsAUCAGUCAUGAGGAUGAGUUUUAGAGCUAGAAAUA | 507 |
| NGA_20nt_4-9_006_ | GCAAGUUAAAAUAAGGCUAGUCCGUUAUCAACUUGAAAAAGUGG | |
| -_31948940_spCas9 | CACCGAGUCGGUGCUsmUsmUsmU | |
| GuideDesign15_CBE_ | mCsmUsmGsACCAUCAGUCAUGAGGAGUUUUAGAGCUAGAAAUA | 508 |
| NGA_20nt_4-9_006_ | GCAAGUUAAAAUAAGGCUAGUCCGUUAUCAACUUGAAAAAGUGG | |
| -_31948943_spCas9 | CACCGAGUCGGUGCUsmUsmUsmU | |
| GuideDesign15_CBE_ | mCsmUsmUsUCAUCUGGCAGAGAAGGGUUUUAGAGCUAGAAAUA | 509 |
| NGA_20nt_4-9_006_ | GCAAGUUAAAAUAAGGCUAGUCCGUUAUCAACUUGAAAAAGUGG | |
| -_31950033_spCas9 | CACCGAGUCGGUGCUsmUsmUsmU | |
| GuideDesign15_CBE_ | mGsmAsmAsUCACCUGCAAGGAGAGGGUUUUAGAGCUAGAAAUA | 510 |
| NGA_20nt_4-9_006_ | GCAAGUUAAAAUAAGGCUAGUCCGUUAUCAACUUGAAAAAGUGG | |
| -_31951538_spCas9 | CACCGAGUCGGUGCUsmUsmUsmU | |
| GuideDesign15_CBE_ | mCsmAsmGsAAUCACCUGCAAGGAGAGUUUUAGAGCUAGAAAUA | 511 |
| NGA_20nt_4-9_006_ | GCAAGUUAAAAUAAGGCUAGUCCGUUAUCAACUUGAAAAAGUGG | |
| -_31951540_spCas9 | CACCGAGUCGGUGCUsmUsmUsmU | |
| GuideDesign15_CBE_ | mUsmCsmAsCCUGCAAGGAGAGGAGAGUUUUAGAGCUAGAAAUA | 512 |
| NGC_20nt_4-9_009_ | GCAAGUUAAAAUAAGGCUAGUCCGUUAUCAACUUGAAAAAGUGG | |
| -_31951535_spCas9 | CACCGAGUCGGUGCUsmUsmUsmU | |
| GuideDesign15_CBE_ | mUsmCsmUsCACCUCUGCAAGUAUUGGUUUUAGAGCUAGAAAUA | 513 |
| NGG_20nt_4-9_003_ | GCAAGUUAAAAUAAGGCUAGUCCGUUAUCAACUUGAAAAAGUGG | |
| -_31951456_spCas9 | CACCGAGUCGGUGCUsmUsmUsmU | |
| GuideDesign15_CBE_ | mCsmUsmCsUCACCUCUGCAAGUAUUGUUUUAGAGCUAGAAAUA | 514 |
| NGG_20nt_4-9_003_ | GCAAGUUAAAAUAAGGCUAGUCCGUUAUCAACUUGAAAAAGUGG | |
| -_31951457_spCas9 | CACCGAGUCGGUGCUsmUsmUsmU | |
| GuideDesign15_CBE_ | mUsmCsmUsCUCACCUCUGCAAGUAUGUUUUAGAGCUAGAAAUA | 515 |
| NGG_20nt_4-9_003_ | GCAAGUUAAAAUAAGGCUAGUCCGUUAUCAACUUGAAAAAGUGG | |
| -_31951458_spCas9 | CACCGAGUCGGUGCUsmUsmUsmU | |
| GuideDesign15_CBE_ | mUsmGsmUsUGUUCCCCUGGGUGCCAGGUUUUAGUACUCUGUAA | 516 |
| NNGRRT_21nt_ | UGAAAAUUACAGAAUCUACUAAAACAAGGCAAAAUGCCGUGUUU | |
| 3-12?_03-12?_- | AUCUCGUCAACUUGUUGGCGAGAUsmUsmUsmU | |
| _31947927_saCas9 | | |
| GuideDesign15_CBE_ | mGsmGsmCsUUUCAUCUGGCAGAGAAGGUUUUAGUACUCUGUAA | 517 |
| NNGRRT_21nt_ | UGAAAAUUACAGAAUCUACUAAAACAAGGCAAAAUGCCGUGUUU | |
| 3-12?_03-12?_- | AUCUCGUCAACUUGUUGGCGAGAUsmUsmUsmU | |
| _31950031_saCas9 | | |
| GuideDesign15_CBE_ | mUsmUsmCsUCUCACCUCUGCAAGUAUGUUUUAGUACUCUGUAA | 518 |
| NNGRRT_21nt_ | UGAAAAUUACAGAAUCUACUAAAACAAGGCAAAAUGCCGUGUUU | |
| 3-12?_03-12?_- | AUCUCGUCAACUUGUUGGCGAGAUsmUsmUsmU | |
| _31951455_saCas9 | | |
| GuideDesign15_CBE_ | mGsmAsmAsAGCUUCGGCCACCUCUUGGUUUUAGUACUCUGUAA | 519 |
| NNNRRT_21nt_ | UGAAAAUUACAGAAUCUACUAAAACAAGGCAAAAUGCCGUGUUU | |
| 3-12?_015_- | AUCUCGUCAACUUGUUGGCGAGAUsmUsmUsmU | |
| _31947758_saCas9 | | |
| GuideDesign15_CBE_ | mUsmGsmUsUGUUCCCCUGGGUGCCAGGUUUUAGUACUCUGUAA | 520 |
| NNNRRT_21nt_ | UGAAAAUUACAGAAUCUACUAAAACAAGGCAAAAUGCCGUGUUU | |
| 3-12?_015_- | AUCUCGUCAACUUGUUGGCGAGAUsmUsmUsmU | |
| _31947927_saCas9 | | |
| GuideDesign15_CBE_ | mGsmUsmCsUGAUCCAUCUAGCACCAGGUUUUAGUACUCUGUAA | 521 |
| NNNRRT_21nt_ | UGAAAAUUACAGAAUCUACUAAAACAAGGCAAAAUGCCGUGUUU | |
| 3-12?_015_- | AUCUCGUCAACUUGUUGGCGAGAUsmUsmUsmU | |
| _31947994_saCas9 | | |
| GuideDesign15_CBE_ | mCsmUsmUsCUGACCAUCAGUCAUGAGGUUUUAGUACUCUGUAA | 522 |
| NNNRRT_21nt_ | UGAAAAUUACAGAAUCUACUAAAACAAGGCAAAAUGCCGUGUUU | |
| 3-12?_015_- | AUCUCGUCAACUUGUUGGCGAGAUsmUsmUsmU | |
| _31948942_saCas9 | | |
| GuideDesign15_CBE_ | mGsmGsmCsUUUCAUCUGGCAGAGAAGGUUUUAGUACUCUGUAA | 523 |
| NNNRRT_21nt_ | UGAAAAUUACAGAAUCUACUAAAACAAGGCAAAAUGCCGUGUUU | |
| 3-12?_015_- | AUCUCGUCAACUUGUUGGCGAGAUsmUsmUsmU | |
| _31950031_saCas9 | | |
| GuideDesign15_CBE_ | mUsmUsmCsUCUCACCUCUGCAAGUAUGUUUUAGUACUCUGUAA | 524 |
| NNNRRT_21nt_ | UGAAAAUUACAGAAUCUACUAAAACAAGGCAAAAUGCCGUGUUU | |
| 3-12?_015_- | AUCUCGUCAACUUGUUGGCGAGAUsmUsmUsmU | |
| _31951455_saCas9 | | |

²“mN” indicates a 2′-OMe modification of the nucleotide “N”, and “Ns” indicates that the nucleotide “N” is linked to the following (i.e., 3′) nucleotide by a phosphorothioate (PS).

TABLE 1C
Representative sequences for guide polynucleotides for
use in guiding a base editor to introduce a missense
mutation to a complement factor B polynucleotide.³

| Guide | | |
|---|---|---|
| poly- | | SEQ |
| nucleotide | | ID |
| name | Guide polynucleotide sequence | NO |
| gRNA1517 | mGsmUsmCsAGGGACUAACACCAAGAGUUUUAGAGCUAGAAAUAGCAAGU | 525 |
| | UAAAAUAAGGCUAGUCCGUUAUCAACUUGAAAAAGUGGCACCGAGUCGGU | |
| | GCUsmUsmUsmU | |
| gRNA1518 | mAsmGsmCsAGCACAUUGUUUCACUGGUUUUAGAGCUAGAAAUAGCAAGU | 526 |
| | UAAAAUAAGGCUAGUCCGUUAUCAACUUGAAAAAGUGGCACCGAGUCGGU | |
| | GCUsmUsmUsmU | |
| gRNA1519 | mUsmGsmUsCUGAUCCAUCUAGCACCGUUUUAGAGCUAGAAAUAGCAAGU | 527 |
| | UAAAAUAAGGCUAGUCCGUUAUCAACUUGAAAAAGUGGCACCGAGUCGGU | |
| | GCUsmUsmUsmU | |
| gRNA1520 | mGsmCsmCsCACCUUCCUUGUGCCAAGUUUUAGAGCUAGAAAUAGCAAGU | 528 |
| | UAAAAUAAGGCUAGUCCGUUAUCAACUUGAAAAAGUGGCACCGAGUCGGU | |
| | GCUsmUsmUsmU | |
| gRNA1521 | mCsmCsmCsACCUUCCUUGUGCCAAUGUUUUAGAGCUAGAAAUAGCAAGU | 529 |
| | UAAAAUAAGGCUAGUCCGUUAUCAACUUGAAAAAGUGGCACCGAGUCGGU | |
| | GCUsmUsmUsmU | |
| gRNA1522 | mCsmCsmAsCCUUCCUUGUGCCAAUGGUUUUAGAGCUAGAAAUAGCAAGU | 530 |
| | UAAAAUAAGGCUAGUCCGUUAUCAACUUGAAAAAGUGGCACCGAGUCGGU | |
| | GCUsmUsmUsmU | |
| gRNA1523 | mCsmGsmGsGCAUCCCCAUUGGCACAGUUUUAGAGCUAGAAAUAGCAAGU | 531 |
| | UAAAAUAAGGCUAGUCCGUUAUCAACUUGAAAAAGUGGCACCGAGUCGGU | |
| | GCUsmUsmUsmU | |
| gRNA1524 | mCsmAsmUsCCCCAUUGGCACAAGGAGUUUUAGAGCUAGAAAUAGCAAGU | 532 |
| | UAAAAUAAGGCUAGUCCGUUAUCAACUUGAAAAAGUGGCACCGAGUCGGU | |
| | GCUsmUsmUsmU | |
| gRNA1525 | mAsmCsmAsGAAGCGGAAGAUCGUCCGUUUUAGAGCUAGAAAUAGCAAGU | 533 |
| | UAAAAUAAGGCUAGUCCGUUAUCAACUUGAAAAAGUGGCACCGAGUCGGU | |
| | GCUsmUsmUsmU | |
| gRNA1526 | mAsmGsmAsUGGAUCAGACAGCAUUGGUUUUAGAGCUAGAAAUAGCAAGU | 534 |
| | UAAAAUAAGGCUAGUCCGUUAUCAACUUGAAAAAGUGGCACCGAGUCGGU | |
| | GCUsmUsmUsmU | |
| gRNA1527 | mAsmCsmGsUCAUAGUCAUAAAAUUCGUUUUAGAGCUAGAAAUAGCAAGU | 535 |
| | UAAAAUAAGGCUAGUCCGUUAUCAACUUGAAAAAGUGGCACCGAGUCGGU | |
| | GCUsmUsmUsmU | |
| gRNA1528 | mUsmUsmCsCGCUUCUGUUGUUCCCCGUUUUAGAGCUAGAAAUAGCAAGU | 536 |
| | UAAAAUAAGGCUAGUCCGUUAUCAACUUGAAAAAGUGGCACCGAGUCGGU | |
| | GCUsmUsmUsmU | |
| gRNA1529 | mGsmCsmAsGCACAUUGUUUCACUGUGUUUUAGAGCUAGAAAUAGCAAGU | 537 |
| | UAAAAUAAGGCUAGUCCGUUAUCAACUUGAAAAAGUGGCACCGAGUCGGU | |
| | GCUsmUsmUsmU | |
| gRNA1530 | mGsmCsmAsCAUUGUUUCACUGUGGAGUUUUAGAGCUAGAAAUAGCAAGU | 538 |
| | UAAAAUAAGGCUAGUCCGUUAUCAACUUGAAAAAGUGGCACCGAGUCGGU | |
| | GCUsmUsmUsmU | |
| gRNA1531 | mGsmCsmCsGCCAGAAUCACCUGCAAGUUUUAGAGCUAGAAAUAGCAAGU | 539 |
| | UAAAAUAAGGCUAGUCCGUUAUCAACUUGAAAAAGUGGCACCGAGUCGGU | |
| | GCUsmUsmUsmU | |
| gRNA1532 | mCsmGsmCsCAGAAUCACCUGCAAGGGUUUUAGAGCUAGAAAUAGCAAGU | 540 |
| | UAAAAUAAGGCUAGUCCGUUAUCAACUUGAAAAAGUGGCACCGAGUCGGU | |
| | GCUsmUsmUsmU | |
| gRNA1533 | mUsmGsmAsUCCAUCUAGCACCAGGUGUUUUAGAGCUAGAAAUAGCAAGU | 541 |
| | UAAAAUAAGGCUAGUCCGUUAUCAACUUGAAAAAGUGGCACCGAGUCGGU | |
| | GCUsmUsmUsmU | |
| gRNA1534 | mGsmGsmGsCAUCCCCAUUGGCACAAGUUUUAGAGCUAGAAAUAGCAAGU | 542 |
| | UAAAAUAAGGCUAGUCCGUUAUCAACUUGAAAAAGUGGCACCGAGUCGGU | |
| | GCUsmUsmUsmU | |
| gRNA1535 | mCsmAsmGsAAGCGGAAGAUCGUCCUGUUUUAGAGCUAGAAAUAGCAAGU | 543 |
| | UAAAAUAAGGCUAGUCCGUUAUCAACUUGAAAAAGUGGCACCGAGUCGGU | |
| | GCUsmUsmUsmU | |
| gRNA1536 | mAsmGsmGsUGAUUCUGGCGGCCCCUGUUUUAGAGCUAGAAAUAGCAAGU | 544 |
| | UAAAAUAAGGCUAGUCCGUUAUCAACUUGAAAAAGUGGCACCGAGUCGGU | |
| | GCUsmUsmUsmU | |
| gRNA1537 | mCsmGsmUsCAUAGUCAUAAAAUUCAGUUUUAGAGCUAGAAAUAGCAAGU | 545 |
| | UAAAAUAAGGCUAGUCCGUUAUCAACUUGAAAAAGUGGCACCGAGUCGGU | |
| | GCUsmUsmUsmU | |
| gRNA1538 | mGsmAsmUsCAGACAGCAUUGGGGCCGUUUUAGAGCUAGAAAUAGCAAGU | 546 |
| | UAAAAUAAGGCUAGUCCGUUAUCAACUUGAAAAAGUGGCACCGAGUCGGU | |
| | GCUsmUsmUsmU | |
| gRNA1539 | mUsmCsmAsGGGACUAACACCAAGAAGUUUUAGAGCUAGAAAUAGCAAGU | 547 |
| | UAAAAUAAGGCUAGUCCGUUAUCAACUUGAAAAAGUGGCACCGAGUCGGU | |
| | GCUsmUsmUsmU | |
| gRNA1540 | mCsmUsmAsUGACGUUGCCCUGAUCAGUUUUAGAGCUAGAAAUAGCAAGU | 548 |
| | UAAAAUAAGGCUAGUCCGUUAUCAACUUGAAAAAGUGGCACCGAGUCGGU | |
| | GCUsmUsmUsmU | |
| gRNA1541 | mAsmCsmAsGCAGCACAUUGUUUCACUGUUUUAGUACUCUGUAAUGAAAA | 549 |
| | UUACAGAAUCUACUAAAACAAGGCAAAAUGCCGUGUUUAUCUCGUCAACU | |
| | UGUUGGCGAGAUsmUsmUsmU | |
| gRNA1542 | mGsmCsmCsCACCUUCCUUGUGCCAAUGUUUUAGUACUCUGUAAUGAAAA | 550 |
| | UUACAGAAUCUACUAAAACAAGGCAAAAUGCCGUGUUUAUCUCGUCAACU | |
| | UGUUGGCGAGAUsmUsmUsmU | |
| gRNA1543 | mAsmAsmCsGUCAUAGUCAUAAAAUUCGUUUUAGUACUCUGUAAUGAAAA | 551 |
| | UUACAGAAUCUACUAAAACAAGGCAAAAUGCCGUGUUUAUCUCGUCAACU | |
| | UGUUGGCGAGAUsmUsmUsmU | |
| gRNA1544 | mUsmCsmUsUCCGCUUCUGUUGUUCCCGUUUUAGUACUCUGUAAUGAAAA | 552 |
| | UUACAGAAUCUACUAAAACAAGGCAAAAUGCCGUGUUUAUCUCGUCAACU | |
| | UGUUGGCGAGAUsmUsmUsmU | |
| gRNA1545 | mCsmGsmUsUCGCCGCUGGGAGCCACGGUUUUAGUACUCUGUAAUGAAAA | 553 |
| | UUACAGAAUCUACUAAAACAAGGCAAAAUGCCGUGUUUAUCUCGUCAACU | |
| | UGUUGGCGAGAUsmUsmUsmU | |
| gRNA1546 | mGsmUsmCsUGAUCCAUCUAGCACCAGGUUUUAGUACUCUGUAAUGAAAA | 554 |
| | UUACAGAAUCUACUAAAACAAGGCAAAAUGCCGUGUUUAUCUCGUCAACU | |
| | UGUUGGCGAGAUsmUsmUsmU | |
| gRNA1547 | mGsmGsmGsCAUCCCCAUUGGCACAAGGUUUUAGUACUCUGUAAUGAAAA | 555 |
| | UUACAGAAUCUACUAAAACAAGGCAAAAUGCCGUGUUUAUCUCGUCAACU | |
| | UGUUGGCGAGAUsmUsmUsmU | |
| gRNA1548 | mAsmGsmGsUGAUUCUGGCGGCCCCUUGUUUUAGUACUCUGUAAUGAAAA | 556 |
| | UUACAGAAUCUACUAAAACAAGGCAAAAUGCCGUGUUUAUCUCGUCAACU | |
| | UGUUGGCGAGAUsmUsmUsmU | |
| gRNA1549 | mCsmUsmAsGAUGGAUCAGACAGCAUGUUUUAGAGCUAGAAAUAGCAAGU | 557 |
| | UAAAAUAAGGCUAGUCCGUUAUCAACUUGAAAAAGUGGCACCGAGUCGGU | |
| | GCUsmUsmUsmU | |
| gRNA1550 | mUsmAsmGsAUGGAUCAGACAGCAUUGUUUUAGAGCUAGAAAUAGCAAGU | 558 |
| | UAAAAUAAGGCUAGUCCGUUAUCAACUUGAAAAAGUGGCACCGAGUCGGU | |
| | GCUsmUsmUsmU | |
| gRNA1551 | mUsmGsmGsCUUUCAUCUGGCAGAGAGUUUUAGAGCUAGAAAUAGCAAGU | 559 |
| | UAAAAUAAGGCUAGUCCGUUAUCAACUUGAAAAAGUGGCACCGAGUCGGU | |
| | GCUsmUsmUsmU | |
| gRNA1552 | mCsmUsmGsUUGUUCCCCUGGGUGCCGUUUUAGAGCUAGAAAUAGCAAGU | 560 |
| | UAAAAUAAGGCUAGUCCGUUAUCAACUUGAAAAAGUGGCACCGAGUCGGU | |
| | GCUsmUsmUsmU | |
| gRNA1553 | mAsmAsmAsGCUUCGGCCACCUCUUGGUUUUAGAGCUAGAAAUAGCAAGU | 561 |
| | UAAAAUAAGGCUAGUCCGUUAUCAACUUGAAAAAGUGGCACCGAGUCGGU | |
| | GCUsmUsmUsmU | |
| gRNA1554 | mAsmAsmGsCUUCGGCCACCUCUUGAGUUUUAGAGCUAGAAAUAGCAAGU | 562 |
| | UAAAAUAAGGCUAGUCCGUUAUCAACUUGAAAAAGUGGCACCGAGUCGGU | |
| | GCUsmUsmUsmU | |
| gRNA1555 | mAsmGsmCsUUCGGCCACCUCUUGAGGUUUUAGAGCUAGAAAUAGCAAGU | 563 |
| | UAAAAUAAGGCUAGUCCGUUAUCAACUUGAAAAAGUGGCACCGAGUCGGU | |
| | GCUsmUsmUsmU | |
| gRNA1556 | mCsmGsmAsAGCUUUCCUGUCUUCCCGUUUUAGAGCUAGAAAUAGCAAGU | 564 |
| | UAAAAUAAGGCUAGUCCGUUAUCAACUUGAAAAAGUGGCACCGAGUCGGU | |
| | GCUsmUsmUsmU | |
| gRNA1557 | mGsmGsmCsUUUCAUCUGGCAGAGAAGUUUUAGAGCUAGAAAUAGCAAGU | 565 |
| | UAAAAUAAGGCUAGUCCGUUAUCAACUUGAAAAAGUGGCACCGAGUCGGU | |
| | GCUsmUsmUsmU | |
| gRNA1558 | mUsmGsmUsUGUUCCCCUGGGUGCCAGUUUUAGAGCUAGAAAUAGCAAGU | 566 |
| | UAAAAUAAGGCUAGUCCGUUAUCAACUUGAAAAAGUGGCACCGAGUCGGU | |
| | GCUsmUsmUsmU | |
| gRNA1559 | mGsmAsmAsAGCUUCGGCCACCUCUUGGUUUUAGUACUCUGUAAUGAAAA | 567 |
| | UUACAGAAUCUACUAAAACAAGGCAAAAUGCCGUGUUUAUCUCGUCAACU | |
| | UGUUGGCGAGAUsmUsmUsmU | |

³“mN” indicates a 2′-OMe modification of the nucleotide “N”, and “Ns” indicates that the nucleotide “N” is linked to the following (i.e., 3′) nucleotide by a phosphorothioate (PS).

TABLE 1D
Representative sequences for guide polynucleotides for use in guiding a base
editor to alter a complement factor B splice site or introduce a new stop codon
into a complement factor B polynucleotide using base editing.⁴

| Guide | | |
|---|---|---|
| polynucleotide | | SEQ ID |
| name | Guide polynucleotide sequence | NO |
| gRNA1181 | mCsmUsmGsCAGGAUUGCACAACAUGGUUUUAGAGCUAGAAAUAGCAAGU | 568 |
| | UAAAAUAAGGCUAGUCCGUUAUCAACUUGAAAAAGUGGCACCGAGUCGGU | |
| | GCUsmUsmUsmU | |
| gRNA1182 | mAsmUsmAsGGCCCAUUUGUCUCCCCGUUUUAGAGCUAGAAAUAGCAAGU | 569 |
| | UAAAAUAAGGCUAGUCCGUUAUCAACUUGAAAAAGUGGCACCGAGUCGGU | |
| | GCUsmUsmUsmU | |
| gRNA1183 | mCsmUsmCsUGCAGGUUGGUGUAAUCGUUUUAGAGCUAGAAAUAGCAAGU | 570 |
| | UAAAAUAAGGCUAGUCCGUUAUCAACUUGAAAAAGUGGCACCGAGUCGGU | |
| | GCUsmUsmUsmU | |
| gRNA1184 | mAsmCsmGsCUCUCACCUGAUAGUCUGUUUUAGAGCUAGAAAUAGCAAGU | 571 |
| | UAAAAUAAGGCUAGUCCGUUAUCAACUUGAAAAAGUGGCACCGAGUCGGU | |
| | GCUsmUsmUsmU | |
| gRNA1185 | mCsmUsmUsACAAUGACUGAGAUCUUGUUUUAGAGCUAGAAAUAGCAAGU | 572 |
| | UAAAAUAAGGCUAGUCCGUUAUCAACUUGAAAAAGUGGCACCGAGUCGGU | |
| | GCUsmUsmUsmU | |
| gRNA1186 | mUsmCsmAsCCUCCGUUGUCACAGAUGUUUUAGAGCUAGAAAUAGCAAGU | 573 |
| | UAAAAUAAGGCUAGUCCGUUAUCAACUUGAAAAAGUGGCACCGAGUCGGU | |
| | GCUsmUsmUsmU | |
| gRNA1187 | mUsmCsmGsCUUACCUCCAGACAAGAGUUUUAGAGCUAGAAAUAGCAAGU | 574 |
| | UAAAAUAAGGCUAGUCCGUUAUCAACUUGAAAAAGUGGCACCGAGUCGGU | |
| | GCUsmUsmUsmU | |
| gRNA1188 | mCsmAsmCsCCACAGGUGGCAAGUUAGUUUUAGAGCUAGAAAUAGCAAGU | 575 |
| | UAAAAUAAGGCUAGUCCGUUAUCAACUUGAAAAAGUGGCACCGAGUCGGU | |
| | GCUsmUsmUsmU | |
| gRNA1189 | mAsmCsmAsGACCACAAGUUGAAGUCGUUUUAGAGCUAGAAAUAGCAAGU | 576 |
| | UAAAAUAAGGCUAGUCCGUUAUCAACUUGAAAAAGUGGCACCGAGUCGGU | |
| | GCUsmUsmUsmU | |
| gRNA1190 | mUsmCsmUsGCAGGAUUGCACAACAUGUUUUAGAGCUAGAAAUAGCAAGU | 577 |
| | UAAAAUAAGGCUAGUCCGUUAUCAACUUGAAAAAGUGGCACCGAGUCGGU | |
| | GCUsmUsmUsmU | |
| gRNA1191 | mGsmCsmAsGGAUUGCACAACAUGGGGUUUUAGAGCUAGAAAUAGCAAGU | 578 |
| | UAAAAUAAGGCUAGUCCGUUAUCAACUUGAAAAAGUGGCACCGAGUCGGU | |
| | GCUsmUsmUsmU | |
| gRNA1192 | mCsmCsmCsUCAGAUGUCUAUGUGUUGUUUUAGAGCUAGAAAUAGCAAGU | 579 |
| | UAAAAUAAGGCUAGUCCGUUAUCAACUUGAAAAAGUGGCACCGAGUCGGU | |
| | GCUsmUsmUsmU | |
| gRNA1193 | mCsmCsmUsCAGAUGUCUAUGUGUUUGUUUUAGAGCUAGAAAUAGCAAGU | 580 |
| | UAAAAUAAGGCUAGUCCGUUAUCAACUUGAAAAAGUGGCACCGAGUCGGU | |
| | GCUsmUsmUsmU | |
| gRNA1194 | mCsmUsmCsAGAUGUCUAUGUGUUUGGUUUUAGAGCUAGAAAUAGCAAGU | 581 |
| | UAAAAUAAGGCUAGUCCGUUAUCAACUUGAAAAAGUGGCACCGAGUCGGU | |
| | GCUsmUsmUsmU | |
| gRNA1195 | mGsmUsmGsUCCCCAGCGCCCUUCAAGUUUUAGAGCUAGAAAUAGCAAGU | 582 |
| | UAAAAUAAGGCUAGUCCGUUAUCAACUUGAAAAAGUGGCACCGAGUCGGU | |
| | GCUsmUsmUsmU | |
| gRNA1196 | mUsmGsmUsCCCCAGCGCCCUUCAAAGUUUUAGAGCUAGAAAUAGCAAGU | 583 |
| | UAAAAUAAGGCUAGUCCGUUAUCAACUUGAAAAAGUGGCACCGAGUCGGU | |
| | GCUsmUsmUsmU | |
| gRNA1197 | mCsmUsmAsCUUCAGGAGGGGAGAAGGUUUUAGAGCUAGAAAUAGCAAGU | 584 |
| | UAAAAUAAGGCUAGUCCGUUAUCAACUUGAAAAAGUGGCACCGAGUCGGU | |
| | GCUsmUsmUsmU | |
| gRNA1198 | mUsmAsmCsUUCAGGAGGGGAGAAGCGUUUUAGAGCUAGAAAUAGCAAGU | 585 |
| | UAAAAUAAGGCUAGUCCGUUAUCAACUUGAAAAAGUGGCACCGAGUCGGU | |
| | GCUsmUsmUsmU | |
| gRNA1199 | mCsmUsmCsCUUGCAGGUGAUUCUGGGUUUUAGAGCUAGAAAUAGCAAGU | 586 |
| | UAAAAUAAGGCUAGUCCGUUAUCAACUUGAAAAAGUGGCACCGAGUCGGU | |
| | GCUsmUsmUsmU | |
| gRNA1200 | mUsmGsmCsAGGUUGGUGUAAUCAGCGUUUUAGAGCUAGAAAUAGCAAGU | 587 |
| | UAAAAUAAGGCUAGUCCGUUAUCAACUUGAAAAAGUGGCACCGAGUCGGU | |
| | GCUsmUsmUsmU | |
| gRNA1201 | mGsmCsmAsGGUUGGUGUAAUCAGCUGUUUUAGAGCUAGAAAUAGCAAGU | 588 |
| | UAAAAUAAGGCUAGUCCGUUAUCAACUUGAAAAAGUGGCACCGAGUCGGU | |
| | GCUsmUsmUsmU | |
| gRNA1202 | mUsmCsmUsCACCUCUGCAAGUAUUGGUUUUAGAGCUAGAAAUAGCAAGU | 589 |
| | UAAAAUAAGGCUAGUCCGUUAUCAACUUGAAAAAGUGGCACCGAGUCGGU | |
| | GCUsmUsmUsmU | |
| gRNA1203 | mCsmUsmUsACUUUGUUGCUGGCAAGGUUUUAGAGCUAGAAAUAGCAAGU | 590 |
| | UAAAAUAAGGCUAGUCCGUUAUCAACUUGAAAAAGUGGCACCGAGUCGGU | |
| | GCUsmUsmUsmU | |
| gRNA1204 | mGsmCsmUsUACAAUGACUGAGAUCUGUUUUAGAGCUAGAAAUAGCAAGU | 591 |
| | UAAAAUAAGGCUAGUCCGUUAUCAACUUGAAAAAGUGGCACCGAGUCGGU | |
| | GCUsmUsmUsmU | |
| gRNA1205 | mUsmCsmAsCCCAGAUAAUCCUCCCUGUUUUAGAGCUAGAAAUAGCAAGU | 592 |
| | UAAAAUAAGGCUAGUCCGUUAUCAACUUGAAAAAGUGGCACCGAGUCGGU | |
| | GCUsmUsmUsmU | |
| gRNA1206 | mCsmCsmUsUCUGACCAUCAGUCAUGGUUUUAGAGCUAGAAAUAGCAAGU | 593 |
| | UAAAAUAAGGCUAGUCCGUUAUCAACUUGAAAAAGUGGCACCGAGUCGGU | |
| | GCUsmUsmUsmU | |
| gRNA1207 | mCsmAsmAsAGGUCACCUUGGCAGGAGUUUUAGAGCUAGAAAUAGCAAGU | 594 |
| | UAAAAUAAGGCUAGUCCGUUAUCAACUUGAAAAAGUGGCACCGAGUCGGU | |
| | GCUsmUsmUsmU | |
| gRNA1208 | mCsmUsmCsGCUUACCUCCAGACAAGGUUUUAGAGCUAGAAAUAGCAAGU | 595 |
| | UAAAAUAAGGCUAGUCCGUUAUCAACUUGAAAAAGUGGCACCGAGUCGGU | |
| | GCUsmUsmUsmU | |
| gRNA1209 | mUsmCsmAsAAGCAAUCCACUGUCCAGUUUUAGAGCUAGAAAUAGCAAGU | 596 |
| | UAAAAUAAGGCUAGUCCGUUAUCAACUUGAAAAAGUGGCACCGAGUCGGU | |
| | GCUsmUsmUsmU | |
| gRNA1210 | mCsmUsmCsCCAGACUCCUUCAUGUAGUUUUAGAGCUAGAAAUAGCAAGU | 597 |
| | UAAAAUAAGGCUAGUCCGUUAUCAACUUGAAAAAGUGGCACCGAGUCGGU | |
| | GCUsmUsmUsmU | |
| gRNA1211 | mCsmCsmCsAGGGGAACAACAGAAGCGUUUUAGAGCUAGAAAUAGCAAGU | 598 |
| | UAAAAUAAGGCUAGUCCGUUAUCAACUUGAAAAAGUGGCACCGAGUCGGU | |
| | GCUsmUsmUsmU | |
| gRNA1212 | mAsmCsmAsGGUGGCAAGUUAUGGUGGUUUUAGAGCUAGAAAUAGCAAGU | 599 |
| | UAAAAUAAGGCUAGUCCGUUAUCAACUUGAAAAAGUGGCACCGAGUCGGU | |
| | GCUsmUsmUsmU | |
| gRNA1213 | mGsmCsmCsAGAUGAAAGCCAGUCUCGUUUUAGAGCUAGAAAUAGCAAGU | 600 |
| | UAAAAUAAGGCUAGUCCGUUAUCAACUUGAAAAAGUGGCACCGAGUCGGU | |
| | GCUsmUsmUsmU | |
| gRNA1214 | mGsmUsmCsCCCAGCGCCCUUCAAAGGUUUUAGAGCUAGAAAUAGCAAGU | 601 |
| | UAAAAUAAGGCUAGUCCGUUAUCAACUUGAAAAAGUGGCACCGAGUCGGU | |
| | GCUsmUsmUsmU | |
| gRNA1215 | mUsmAsmCsAGAAAGGCAGCUGUGAGGUUUUAGAGCUAGAAAUAGCAAGU | 602 |
| | UAAAAUAAGGCUAGUCCGUUAUCAACUUGAAAAAGUGGCACCGAGUCGGU | |
| | GCUsmUsmUsmU | |
| gRNA1216 | mGsmAsmGsGACUCACUUGAAUGAAAGUUUUAGAGCUAGAAAUAGCAAGU | 603 |
| | UAAAAUAAGGCUAGUCCGUUAUCAACUUGAAAAAGUGGCACCGAGUCGGU | |
| | GCUsmUsmUsmU | |
| gRNA1217 | mUsmCsmUsCACCUUAUCCCCAUUCUGUUUUAGAGCUAGAAAUAGCAAGU | 604 |
| | UAAAAUAAGGCUAGUCCGUUAUCAACUUGAAAAAGUGGCACCGAGUCGGU | |
| | GCUsmUsmUsmU | |
| gRNA1218 | mAsmUsmCsCUUACCUACGCUGACCUGUUUUAGAGCUAGAAAUAGCAAGU | 605 |
| | UAAAAUAAGGCUAGUCCGUUAUCAACUUGAAAAAGUGGCACCGAGUCGGU | |
| | GCUsmUsmUsmU | |
| gRNA1219 | mUsmUsmAsCCUACGCUGACCUUGAUGUUUUAGAGCUAGAAAUAGCAAGU | 606 |
| | UAAAAUAAGGCUAGUCCGUUAUCAACUUGAAAAAGUGGCACCGAGUCGGU | |
| | GCUsmUsmUsmU | |
| gRNA1220 | mCsmUsmCsCCUACCGAUCAUUUGGUGUUUUAGAGCUAGAAAUAGCAAGU | 607 |
| | UAAAAUAAGGCUAGUCCGUUAUCAACUUGAAAAAGUGGCACCGAGUCGGU | |
| | GCUsmUsmUsmU | |
| gRNA1221 | mCsmUsmGsACCAUCAGUCAUGAGGAGUUUUAGAGCUAGAAAUAGCAAGU | 608 |
| | UAAAAUAAGGCUAGUCCGUUAUCAACUUGAAAAAGUGGCACCGAGUCGGU | |
| | GCUsmUsmUsmU | |
| gRNA1222 | mCsmUsmUsCUCACCUCCGUUGUCACGUUUUAGAGCUAGAAAUAGCAAGU | 609 |
| | UAAAAUAAGGCUAGUCCGUUAUCAACUUGAAAAAGUGGCACCGAGUCGGU | |
| | GCUsmUsmUsmU | |
| gRNA1223 | mUsmCsmCsCUCAGAUGUCUAUGUGUUGUUUUAGUACUCUGUAAUGAAAA | 610 |
| | UUACAGAAUCUACUAAAACAAGGCAAAAUGCCGUGUUUAUCUCGUCAACU | |
| | UGUUGGCGAGAUsmUsmUsmU | |
| gRNA1224 | mCsmUsmGsCCAGAUGAAAGCCAGUCUGUUUUAGUACUCUGUAAUGAAAA | 611 |
| | UUACAGAAUCUACUAAAACAAGGCAAAAUGCCGUGUUUAUCUCGUCAACU | |
| | UGUUGGCGAGAUsmUsmUsmU | |
| gRNA1225 | mUsmUsmCsUCUCACCUCUGCAAGUAUGUUUUAGUACUCUGUAAUGAAAA | 612 |
| | UUACAGAAUCUACUAAAACAAGGCAAAAUGCCGUGUUUAUCUCGUCAACU | |
| | UGUUGGCGAGAUsmUsmUsmU | |
| gRNA1226 | mCsmCsmUsUACCUACGCUGACCUUGAGUUUUAGUACUCUGUAAUGAAAA | 613 |
| | UUACAGAAUCUACUAAAACAAGGCAAAAUGCCGUGUUUAUCUCGUCAACU | |
| | UGUUGGCGAGAUsmUsmUsmU | |
| gRNA1227 | mAsmCsmUsCACCCAGAUAAUCCUCCCGUUUUAGUACUCUGUAAUGAAAA | 614 |
| | UUACAGAAUCUACUAAAACAAGGCAAAAUGCCGUGUUUAUCUCGUCAACU | |
| | UGUUGGCGAGAUsmUsmUsmU | |
| gRNA1228 | mUsmCsmCsCUUCUGACCAUCAGUCAUGUUUUAGUACUCUGUAAUGAAAA | 615 |
| | UUACAGAAUCUACUAAAACAAGGCAAAAUGCCGUGUUUAUCUCGUCAACU | |
| | UGUUGGCGAGAUsmUsmUsmU | |
| gRNA1229 | mCsmUsmUsCAGGUGUGACCACCACUCGUUUUAGUACUCUGUAAUGAAAA | 616 |
| | UUACAGAAUCUACUAAAACAAGGCAAAAUGCCGUGUUUAUCUCGUCAACU | |
| | UGUUGGCGAGAUsmUsmUsmU | |
| gRNA1230 | mCsmCsmCsUCUCCACAGACCACAAGUGUUUUAGUACUCUGUAAUGAAAA | 617 |
| | UUACAGAAUCUACUAAAACAAGGCAAAAUGCCGUGUUUAUCUCGUCAACU | |
| | UGUUGGCGAGAUsmUsmUsmU | |
| gRNA1231 | mCsmUsmAsCAGAAAGGCAGCUGUGAGGUUUUAGUACUCUGUAAUGAAAA | 618 |
| | UUACAGAAUCUACUAAAACAAGGCAAAAUGCCGUGUUUAUCUCGUCAACU | |
| | UGUUGGCGAGAUsmUsmUsmU | |
| gRNA1232 | mGsmUsmAsUGUCUUACUUUGUUGCUGGUUUUAGUACUCUGUAAUGAAAA | 619 |
| | UUACAGAAUCUACUAAAACAAGGCAAAAUGCCGUGUUUAUCUCGUCAACU | |
| | UGUUGGCGAGAUsmUsmUsmU | |
| gRNA1233 | mUsmGsmUsCUUACUUUGUUGCUGGCAGUUUUAGUACUCUGUAAUGAAAA | 620 |
| | UUACAGAAUCUACUAAAACAAGGCAAAAUGCCGUGUUUAUCUCGUCAACU | |
| | UGUUGGCGAGAUsmUsmUsmU | |
| gRNA1234 | mUsmGsmCsAUCCUUACCUACGCUGACGUUUUAGUACUCUGUAAUGAAAA | 621 |
| | UUACAGAAUCUACUAAAACAAGGCAAAAUGCCGUGUUUAUCUCGUCAACU | |
| | UGUUGGCGAGAUsmUsmUsmU | |
| gRNA1235 | mGsmGsmAsUUCUGUGCUUACAAUGACGUUUUAGUACUCUGUAAUGAAAA | 622 |
| | UUACAGAAUCUACUAAAACAAGGCAAAAUGCCGUGUUUAUCUCGUCAACU | |
| | UGUUGGCGAGAUsmUsmUsmU | |
| gRNA1236 | mCsmUsmUsGUAUCUCCCUACCGAUCAGUUUUAGUACUCUGUAAUGAAAA | 623 |
| | UUACAGAAUCUACUAAAACAAGGCAAAAUGCCGUGUUUAUCUCGUCAACU | |
| | UGUUGGCGAGAUsmUsmUsmU | |
| gRNA1237 | mCsmUsmUsCUGACCAUCAGUCAUGAGGUUUUAGUACUCUGUAAUGAAAA | 624 |
| | UUACAGAAUCUACUAAAACAAGGCAAAAUGCCGUGUUUAUCUCGUCAACU | |
| | UGUUGGCGAGAUsmUsmUsmU | |
| gRNA1238 | mCsmCsmCsUAACCUCUGACCUUCAUAGUUUUAGUACUCUGUAAUGAAAA | 625 |
| | UUACAGAAUCUACUAAAACAAGGCAAAAUGCCGUGUUUAUCUCGUCAACU | |
| | UGUUGGCGAGAUsmUsmUsmU | |
| gRNA1239 | mAsmGsmGsAGGAUUCCACCUUCUCAAGUUUUAGUACUCUGUAAUGAAAA | 626 |
| | UUACAGAAUCUACUAAAACAAGGCAAAAUGCCGUGUUUAUCUCGUCAACU | |
| | UGUUGGCGAGAUsmUsmUsmU | |
| gRNA1240 | mAsmUsmGsCUUCUCACCUCCGUUGUCGUUUUAGUACUCUGUAAUGAAAA | 627 |
| | UUACAGAAUCUACUAAAACAAGGCAAAAUGCCGUGUUUAUCUCGUCAACU | |
| | UGUUGGCGAGAUsmUsmUsmU | |
| gRNA1241 | mUsmUsmCsUCCAAGAGGGCCAGGCACGUUUUAGUACUCUGUAAUGAAAA | 628 |
| | UUACAGAAUCUACUAAAACAAGGCAAAAUGCCGUGUUUAUCUCGUCAACU | |
| | UGUUGGCGAGAUsmUsmUsmU | |
| gRNA1242 | mAsmCsmCsAAAAGACUGUCAGGAAGGGUUUUAGUACUCUGUAAUGAAAA | 629 |
| | UUACAGAAUCUACUAAAACAAGGCAAAAUGCCGUGUUUAUCUCGUCAACU | |
| | UGUUGGCGAGAUsmUsmUsmU | |
| gRNA1243 | mAsmCsmCsUGCCAAGUGAAUGGCCGAGUUUUAGUACUCUGUAAUGAAAA | 630 |
| | UUACAGAAUCUACUAAAACAAGGCAAAAUGCCGUGUUUAUCUCGUCAACU | |
| | UGUUGGCGAGAUsmUsmUsmU | |
| gRNA1244 | mGsmAsmGsCAACAUGUGUUCAAAGUCGUUUUAGUACUCUGUAAUGAAAA | 631 |
| | UUACAGAAUCUACUAAAACAAGGCAAAAUGCCGUGUUUAUCUCGUCAACU | |
| | UGUUGGCGAGAUsmUsmUsmU | |
| gRNA1245 | mUsmAsmCsACCAACCUGCAGAGGCAGGUUUUAGUACUCUGUAAUGAAAA | 632 |
| | UUACAGAAUCUACUAAAACAAGGCAAAAUGCCGUGUUUAUCUCGUCAACU | |
| | UGUUGGCGAGAUsmUsmUsmU | |
| gRNA1246 | mGsmGsmCsUUUCAUCUGGCAGAGAAGGUUUUAGUACUCUGUAAUGAAAA | 633 |
| | UUACAGAAUCUACUAAAACAAGGCAAAAUGCCGUGUUUAUCUCGUCAACU | |
| | UGUUGGCGAGAUsmUsmUsmU | |
| gRNA1247 | mGsmAsmCsAUCUGAGGGAUAAAAAGGGUUUUAGUACUCUGUAAUGAAAA | 634 |
| | UUACAGAAUCUACUAAAACAAGGCAAAAUGCCGUGUUUAUCUCGUCAACU | |
| | UGUUGGCGAGAUsmUsmUsmU | |
| gRNA1248 | mUsmGsmUsUGUUCCCCUGGGUGCCAGGUUUUAGUACUCUGUAAUGAAAA | 635 |
| | UUACAGAAUCUACUAAAACAAGGCAAAAUGCCGUGUUUAUCUCGUCAACU | |
| | UGUUGGCGAGAUsmUsmUsmU | |
| gRNA1249 | mAsmUsmCsGCACCUGCCAAGUGAAUGGUUUUAGUACUCUGUAAUGAAAA | 636 |
| | UUACAGAAUCUACUAAAACAAGGCAAAAUGCCGUGUUUAUCUCGUCAACU | |
| | UGUUGGCGAGAUsmUsmUsmU | |
| gRNA1250 | mUsmGsmGsCCGAUGGAGUGGGCAGACGUUUUAGUACUCUGUAAUGAAAA | 637 |
| | UUACAGAAUCUACUAAAACAAGGCAAAAUGCCGUGUUUAUCUCGUCAACU | |
| | UGUUGGCGAGAUsmUsmUsmU | |
| gRNA1251 | mUsmCsmCsCAGCGGCGAACGUGUCAGGUUUUAGUACUCUGUAAUGAAAA | 638 |
| | UUACAGAAUCUACUAAAACAAGGCAAAAUGCCGUGUUUAUCUCGUCAACU | |
| | UGUUGGCGAGAUsmUsmUsmU | |
| gRNA1252 | mCsmCsmCsAGGGGAACAACAGAAGCGGUUUUAGUACUCUGUAAUGAAAA | 639 |
| | UUACAGAAUCUACUAAAACAAGGCAAAAUGCCGUGUUUAUCUCGUCAACU | |
| | UGUUGGCGAGAUsmUsmUsmU | |
| gRNA1253 | mGsmUsmCsACGAAGCAGCUCAAUGAAGUUUUAGUACUCUGUAAUGAAAA | 640 |
| | UUACAGAAUCUACUAAAACAAGGCAAAAUGCCGUGUUUAUCUCGUCAACU | |
| | UGUUGGCGAGAUsmUsmUsmU | |
| gRNA1254 | mGsmGsmCsCCUCCAGGCAGUGUACAGGUUUUAGUACUCUGUAAUGAAAA | 641 |
| | UUACAGAAUCUACUAAAACAAGGCAAAAUGCCGUGUUUAUCUCGUCAACU | |
| | UGUUGGCGAGAUsmUsmUsmU | |
| gRNA1255 | mUsmUsmUsCUACCAAAUGAUCGGUAGGUUUUAGUACUCUGUAAUGAAAA | 642 |
| | UUACAGAAUCUACUAAAACAAGGCAAAAUGCCGUGUUUAUCUCGUCAACU | |
| | UGUUGGCGAGAUsmUsmUsmU | |
| gRNA1256 | mGsmCsmAsACCAUGGCAGGCCAAGAUGUUUUAGUACUCUGUAAUGAAAA | 643 |
| | UUACAGAAUCUACUAAAACAAGGCAAAAUGCCGUGUUUAUCUCGUCAACU | |
| | UGUUGGCGAGAUsmUsmUsmU | |
| gRNA1257 | mUsmCsmAsGCCAGGGCAGCACUUGAAGUUUUAGUACUCUGUAAUGAAAA | 644 |
| | UUACAGAAUCUACUAAAACAAGGCAAAAUGCCGUGUUUAUCUCGUCAACU | |
| | UGUUGGCGAGAUsmUsmUsmU | |
| gRNA1258 | mCsmUsmUsACUUUGUUGCUGGCAAGUGUUUUAGUACUCUGUAAUGAAAA | 645 |
| | UUACAGAAUCUACUAAAACAAGGCAAAAUGCCGUGUUUAUCUCGUCAACU | |
| | UGUUGGCGAGAUsmUsmUsmU | |
| gRNA1259 | mCsmUsmCsCCCUCCUGAAGUAGGAGAGUUUUAGUACUCUGUAAUGAAAA | 646 |
| | UUACAGAAUCUACUAAAACAAGGCAAAAUGCCGUGUUUAUCUCGUCAACU | |
| | UGUUGGCGAGAUsmUsmUsmU | |
| gRNA1260 | mUsmUsmUsGAAGGGCGCUGGGGACACGUUUUAGUACUCUGUAAUGAAAA | 647 |
| | UUACAGAAUCUACUAAAACAAGGCAAAAUGCCGUGUUUAUCUCGUCAACU | |
| | UGUUGGCGAGAUsmUsmUsmU | |
| gRNA1261 | mUsmCsmUsUGGCCUGCCAUGGUUGCUGUUUUAGUACUCUGUAAUGAAAA | 648 |
| | UUACAGAAUCUACUAAAACAAGGCAAAAUGCCGUGUUUAUCUCGUCAACU | |
| | UGUUGGCGAGAUsmUsmUsmU | |
| gRNA1262 | mUsmGsmGsCCUGCCAUGGUUGCUUGUGUUUUAGUACUCUGUAAUGAAAA | 649 |
| | UUACAGAAUCUACUAAAACAAGGCAAAAUGCCGUGUUUAUCUCGUCAACU | |
| | UGUUGGCGAGAUsmUsmUsmU | |
| gRNA1263 | mAsmUsmGsAAGGAGUCUGGGAGAGUCGUUUUAGUACUCUGUAAUGAAAA | 650 |
| | UUACAGAAUCUACUAAAACAAGGCAAAAUGCCGUGUUUAUCUCGUCAACU | |
| | UGUUGGCGAGAUsmUsmUsmU | |
| gRNA1264 | mGsmGsmAsGUCUGGGAGAGUCAGAAAGUUUUAGUACUCUGUAAUGAAAA | 651 |
| | UUACAGAAUCUACUAAAACAAGGCAAAAUGCCGUGUUUAUCUCGUCAACU | |
| | UGUUGGCGAGAUsmUsmUsmU | |
| gRNA1265 | mCsmGsmCsUGCAGAGGUAUGAGACAUGUUUUAGUACUCUGUAAUGAAAA | 652 |
| | UUACAGAAUCUACUAAAACAAGGCAAAAUGCCGUGUUUAUCUCGUCAACU | |
| | UGUUGGCGAGAUsmUsmUsmU | |
| gRNA1266 | mAsmCsmUsCCAUCGGCCAUUCACUUGGUUUUAGUACUCUGUAAUGAAAA | 653 |
| | UUACAGAAUCUACUAAAACAAGGCAAAAUGCCGUGUUUAUCUCGUCAACU | |
| | UGUUGGCGAGAUsmUsmUsmU | |
| gRNA1267 | mGsmGsmGsGCCAGUAUUCCCCGUUCUGUUUUAGUACUCUGUAAUGAAAA | 654 |
| | UUACAGAAUCUACUAAAACAAGGCAAAAUGCCGUGUUUAUCUCGUCAACU | |
| | UGUUGGCGAGAUsmUsmUsmU | |
| gRNA1268 | mGsmGsmAsUUGCUUUGAGAAGGGGGGGUUUUAGUACUCUGUAAUGAAAA | 655 |
| | UUACAGAAUCUACUAAAACAAGGCAAAAUGCCGUGUUUAUCUCGUCAACU | |
| | UGUUGGCGAGAUsmUsmUsmU | |
| gRNA1269 | mUsmUsmCsAGGGUGCUCCAGGACCCCGUUUUAGUACUCUGUAAUGAAAA | 656 |
| | UUACAGAAUCUACUAAAACAAGGCAAAAUGCCGUGUUUAUCUCGUCAACU | |
| | UGUUGGCGAGAUsmUsmUsmU | |
| gRNA1270 | mGsmCsmUsCCAGGACCCCGUAGAUCUGUUUUAGUACUCUGUAAUGAAAA | 657 |
| | UUACAGAAUCUACUAAAACAAGGCAAAAUGCCGUGUUUAUCUCGUCAACU | |
| | UGUUGGCGAGAUsmUsmUsmU | |
| gRNA1271 | mUsmGsmGsUCACACCUGAAGAGAAAGGUUUUAGUACUCUGUAAUGAAAA | 658 |
| | UUACAGAAUCUACUAAAACAAGGCAAAAUGCCGUGUUUAUCUCGUCAACU | |
| | UGUUGGCGAGAUsmUsmUsmU | |
| gRNA1272 | mCsmUsmUsACCUCCAGACAAGAGGCCGUUUUAGUACUCUGUAAUGAAAA | 659 |
| | UUACAGAAUCUACUAAAACAAGGCAAAAUGCCGUGUUUAUCUCGUCAACU | |
| | UGUUGGCGAGAUsmUsmUsmU | |
| gRNA1356 | mCsmUsmCsCUUCCGACUUCUCCAAGGUUUUAGAGCUAGAAAUAGCAAGU | 660 |
| | UAAAAUAAGGCUAGUCCGUUAUCAACUUGAAAAAGUGGCACCGAGUCGGU | |
| | GCUsmUsmUsmU | |
| gRNA1357 | mUsmCsmCsUUCCGACUUCUCCAAGAGUUUUAGAGCUAGAAAUAGCAAGU | 661 |
| | UAAAAUAAGGCUAGUCCGUUAUCAACUUGAAAAAGUGGCACCGAGUCGGU | |
| | GCUsmUsmUsmU | |
| gRNA1358 | mUsmCsmUsCCAAGAGGGCCAGGCACGUUUUAGAGCUAGAAAUAGCAAGU | 662 |
| | UAAAAUAAGGCUAGUCCGUUAUCAACUUGAAAAAGUGGCACCGAGUCGGU | |
| | GCUsmUsmUsmU | |
| gRNA1359 | mCsmUsmCsAAGACCAAAAGACUGUCGUUUUAGAGCUAGAAAUAGCAAGU | 663 |
| | UAAAAUAAGGCUAGUCCGUUAUCAACUUGAAAAAGUGGCACCGAGUCGGU | |
| | GCUsmUsmUsmU | |
| gRNA1360 | mAsmGsmAsCCAAAAGACUGUCAGGAGUUUUAGAGCUAGAAAUAGCAAGU | 664 |
| | UAAAAUAAGGCUAGUCCGUUAUCAACUUGAAAAAGUGGCACCGAGUCGGU | |
| | GCUsmUsmUsmU | |
| gRNA1361 | mCsmCsmUsGCCAAGUGAAUGGCCGAGUUUUAGAGCUAGAAAUAGCAAGU | 665 |
| | UAAAAUAAGGCUAGUCCGUUAUCAACUUGAAAAAGUGGCACCGAGUCGGU | |
| | GCUsmUsmUsmU | |
| gRNA1362 | mCsmUsmCsCCAGCGGCGAACGUGUCGUUUUAGAGCUAGAAAUAGCAAGU | 666 |
| | UAAAAUAAGGCUAGUCCGUUAUCAACUUGAAAAAGUGGCACCGAGUCGGU | |
| | GCUsmUsmUsmU | |
| gRNA1363 | mCsmAsmGsCGGCGAACGUGUCAGGAGUUUUAGAGCUAGAAAUAGCAAGU | 667 |
| | UAAAAUAAGGCUAGUCCGUUAUCAACUUGAAAAAGUGGCACCGAGUCGGU | |
| | GCUsmUsmUsmU | |
| gRNA1364 | mCsmGsmGsCGAACGUGUCAGGAAGGGUUUUAGAGCUAGAAAUAGCAAGU | 668 |
| | UAAAAUAAGGCUAGUCCGUUAUCAACUUGAAAAAGUGGCACCGAGUCGGU | |
| | GCUsmUsmUsmU | |
| gRNA1365 | mCsmGsmUsGUCAGGAAGGUGGCUCUGUUUUAGAGCUAGAAAUAGCAAGU | 669 |
| | UAAAAUAAGGCUAGUCCGUUAUCAACUUGAAAAAGUGGCACCGAGUCGGU | |
| | GCUsmUsmUsmU | |
| gRNA1366 | mUsmUsmUsUCUACCAAAUGAUCGGUGUUUUAGAGCUAGAAAUAGCAAGU | 670 |
| | UAAAAUAAGGCUAGUCCGUUAUCAACUUGAAAAAGUGGCACCGAGUCGGU | |
| | GCUsmUsmUsmU | |
| gRNA1367 | mUsmUsmUsCUACCAAAUGAUCGGUAGUUUUAGAGCUAGAAAUAGCAAGU | 671 |
| | UAAAAUAAGGCUAGUCCGUUAUCAACUUGAAAAAGUGGCACCGAGUCGGU | |
| | GCUsmUsmUsmU | |
| gRNA1368 | mGsmCsmAsACAAAGUAAGACAUACUGUUUUAGAGCUAGAAAUAGCAAGU | 672 |
| | UAAAAUAAGGCUAGUCCGUUAUCAACUUGAAAAAGUGGCACCGAGUCGGU | |
| | GCUsmUsmUsmU | |
| gRNA1369 | mAsmCsmCsUCUUUCAAGUGCUGCCCGUUUUAGAGCUAGAAAUAGCAAGU | 673 |
| | UAAAAUAAGGCUAGUCCGUUAUCAACUUGAAAAAGUGGCACCGAGUCGGU | |
| | GCUsmUsmUsmU | |
| gRNA1370 | mGsmAsmAsACUCCAAGAUGAGGAUUGUUUUAGAGCUAGAAAUAGCAAGU | 674 |
| | UAAAAUAAGGCUAGUCCGUUAUCAACUUGAAAAAGUGGCACCGAGUCGGU | |
| | GCUsmUsmUsmU | |
| gRNA1371 | mAsmAsmAsCUCCAAGAUGAGGAUUUGUUUUAGAGCUAGAAAUAGCAAGU | 675 |
| | UAAAAUAAGGCUAGUCCGUUAUCAACUUGAAAAAGUGGCACCGAGUCGGU | |
| | GCUsmUsmUsmU | |
| gRNA1372 | mCsmAsmCsCAACCUGCAGAGGCAGUGUUUUAGAGCUAGAAAUAGCAAGU | 676 |
| | UAAAAUAAGGCUAGUCCGUUAUCAACUUGAAAAAGUGGCACCGAGUCGGU | |
| | GCUsmUsmUsmU | |
| gRNA1373 | mAsmCsmCsAACCUGCAGAGGCAGUGGUUUUAGAGCUAGAAAUAGCAAGU | 677 |
| | UAAAAUAAGGCUAGUCCGUUAUCAACUUGAAAAAGUGGCACCGAGUCGGU | |
| | GCUsmUsmUsmU | |
| gRNA1374 | mUsmGsmCsCUUUCUGUAGGGGAGGUGUUUUAGAGCUAGAAAUAGCAAGU | 678 |
| | UAAAAUAAGGCUAGUCCGUUAUCAACUUGAAAAAGUGGCACCGAGUCGGU | |
| | GCUsmUsmUsmU | |
| gRNA1375 | mAsmAsmAsUGGGCCUAUAAAGGACAGUUUUAGAGCUAGAAAUAGCAAGU | 679 |
| | UAAAAUAAGGCUAGUCCGUUAUCAACUUGAAAAAGUGGCACCGAGUCGGU | |
| | GCUsmUsmUsmU | |
| gRNA1376 | mCsmCsmCsUCCUGAAGUAGGAGAGUGUUUUAGAGCUAGAAAUAGCAAGU | 680 |
| | UAAAAUAAGGCUAGUCCGUUAUCAACUUGAAAAAGUGGCACCGAGUCGGU | |
| | GCUsmUsmUsmU | |
| gRNA1377 | mUsmGsmGsCCUGCCAUGGUUGCUUGGUUUUAGAGCUAGAAAUAGCAAGU | 681 |
| | UAAAAUAAGGCUAGUCCGUUAUCAACUUGAAAAAGUGGCACCGAGUCGGU | |
| | GCUsmUsmUsmU | |
| gRNA1378 | mUsmAsmGsACAUCUGAGGGAUAAAAGUUUUAGAGCUAGAAAUAGCAAGU | 682 |
| | UAAAAUAAGGCUAGUCCGUUAUCAACUUGAAAAAGUGGCACCGAGUCGGU | |
| | GCUsmUsmUsmU | |
| gRNA1379 | mCsmAsmUsCUGAGGGAUAAAAAGGAGUUUUAGAGCUAGAAAUAGCAAGU | 683 |
| | UAAAAUAAGGCUAGUCCGUUAUCAACUUGAAAAAGUGGCACCGAGUCGGU | |
| | GCUsmUsmUsmU | |
| gRNA1380 | mUsmGsmCsAAUCCUGCAGAAGAGACGUUUUAGAGCUAGAAAUAGCAAGU | 684 |
| | UAAAAUAAGGCUAGUCCGUUAUCAACUUGAAAAAGUGGCACCGAGUCGGU | |
| | GCUsmUsmUsmU | |
| gRNA1381 | mGsmUsmGsCGGUUCCAGCCUUCAGGGUUUUAGAGCUAGAAAUAGCAAGU | 685 |
| | UAAAAUAAGGCUAGUCCGUUAUCAACUUGAAAAAGUGGCACCGAGUCGGU | |
| | GCUsmUsmUsmU | |
| gRNA1382 | mUsmGsmCsGGUUCCAGCCUUCAGGAGUUUUAGAGCUAGAAAUAGCAAGU | 686 |
| | UAAAAUAAGGCUAGUCCGUUAUCAACUUGAAAAAGUGGCACCGAGUCGGU | |
| | GCUsmUsmUsmU | |
| gRNA1383 | mGsmAsmCsCCAAAUUUUGGGGUAUGGUUUUAGAGCUAGAAAUAGCAAGU | 687 |
| | UAAAAUAAGGCUAGUCCGUUAUCAACUUGAAAAAGUGGCACCGAGUCGGU | |
| | GCUsmUsmUsmU | |
| gRNA1384 | mCsmAsmCsUCCAUCGGCCAUUCACUGUUUUAGAGCUAGAAAUAGCAAGU | 688 |
| | UAAAAUAAGGCUAGUCCGUUAUCAACUUGAAAAAGUGGCACCGAGUCGGU | |
| | GCUsmUsmUsmU | |
| gRNA1385 | mGsmUsmGsGAUUGCUUUGAGAAGGGGUUUUAGAGCUAGAAAUAGCAAGU | 689 |
| | UAAAAUAAGGCUAGUCCGUUAUCAACUUGAAAAAGUGGCACCGAGUCGGU | |
| | GCUsmUsmUsmU | |
| gRNA1386 | mCsmCsmCsCAGGGAUCCUGCUCUCUGUUUUAGAGCUAGAAAUAGCAAGU | 690 |
| | UAAAAUAAGGCUAGUCCGUUAUCAACUUGAAAAAGUGGCACCGAGUCGGU | |
| | GCUsmUsmUsmU | |
| gRNA1387 | mUsmCsmAsAGACCAAAAGACUGUCAGUUUUAGAGCUAGAAAUAGCAAGU | 691 |
| | UAAAAUAAGGCUAGUCCGUUAUCAACUUGAAAAAGUGGCACCGAGUCGGU | |
| | GCUsmUsmUsmU | |
| gRNA1388 | mGsmCsmAsCCUGCCAAGUGAAUGGCGUUUUAGAGCUAGAAAUAGCAAGU | 692 |
| | UAAAAUAAGGCUAGUCCGUUAUCAACUUGAAAAAGUGGCACCGAGUCGGU | |
| | GCUsmUsmUsmU | |
| gRNA1389 | mCsmUsmGsCCAAGUGAAUGGCCGAUGUUUUAGAGCUAGAAAUAGCAAGU | 693 |
| | UAAAAUAAGGCUAGUCCGUUAUCAACUUGAAAAAGUGGCACCGAGUCGGU | |
| | GCUsmUsmUsmU | |
| gRNA1390 | mGsmAsmAsUGGCCGAUGGAGUGGGCGUUUUAGAGCUAGAAAUAGCAAGU | 694 |
| | UAAAAUAAGGCUAGUCCGUUAUCAACUUGAAAAAGUGGCACCGAGUCGGU | |
| | GCUsmUsmUsmU | |
| gRNA1391 | mAsmGsmUsGGGCAGACAGCGAUCUGGUUUUAGAGCUAGAAAUAGCAAGU | 695 |
| | UAAAAUAAGGCUAGUCCGUUAUCAACUUGAAAAAGUGGCACCGAGUCGGU | |
| | GCUsmUsmUsmU | |
| gRNA1392 | mGsmGsmCsAGCCAGUACCGCCUUGAGUUUUAGAGCUAGAAAUAGCAAGU | 696 |
| | UAAAAUAAGGCUAGUCCGUUAUCAACUUGAAAAAGUGGCACCGAGUCGGU | |
| | GCUsmUsmUsmU | |
| gRNA1393 | mUsmCsmCsCAGCGGCGAACGUGUCAGUUUUAGAGCUAGAAAUAGCAAGU | 697 |
| | UAAAAUAAGGCUAGUCCGUUAUCAACUUGAAAAAGUGGCACCGAGUCGGU | |
| | GCUsmUsmUsmU | |
| gRNA1394 | mGsmUsmGsUCAGGAAGGUGGCUCUUGUUUUAGAGCUAGAAAUAGCAAGU | 698 |
| | UAAAAUAAGGCUAGUCCGUUAUCAACUUGAAAAAGUGGCACCGAGUCGGU | |
| | GCUsmUsmUsmU | |
| gRNA1395 | mAsmGsmGsGGAACAACAGAAGCGGAGUUUUAGAGCUAGAAAUAGCAAGU | 699 |
| | UAAAAUAAGGCUAGUCCGUUAUCAACUUGAAAAAGUGGCACCGAGUCGGU | |
| | GCUsmUsmUsmU | |
| gRNA1396 | mCsmCsmUsCCAGGCAGUGUACAGCAGUUUUAGAGCUAGAAAUAGCAAGU | 700 |
| | UAAAAUAAGGCUAGUCCGUUAUCAACUUGAAAAAGUGGCACCGAGUCGGU | |
| | GCUsmUsmUsmU | |
| gRNA1397 | mUsmUsmCSUACCAAAUGAUCGGUAGGUUUUAGAGCUAGAAAUAGCAAGU | 701 |
| | UAAAAUAAGGCUAGUCCGUUAUCAACUUGAAAAAGUGGCACCGAGUCGGU | |
| | GCUsmUsmUsmU | |
| gRNA1398 | mCsmUsmAsCCAAAUGAUCGGUAGGGGUUUUAGAGCUAGAAAUAGCAAGU | 702 |
| | UAAAAUAAGGCUAGUCCGUUAUCAACUUGAAAAAGUGGCACCGAGUCGGU | |
| | GCUsmUsmUsmU | |
| gRNA1399 | mCsmAsmAsGCAACCAUGGCAGGCCAGUUUUAGAGCUAGAAAUAGCAAGU | 703 |
| | UAAAAUAAGGCUAGUCCGUUAUCAACUUGAAAAAGUGGCACCGAGUCGGU | |
| | GCUsmUsmUsmU | |
| gRNA1400 | mUsmAsmUsGGCCAGACUAUCAGGUGGUUUUAGAGCUAGAAAUAGCAAGU | 704 |
| | UAAAAUAAGGCUAGUCCGUUAUCAACUUGAAAAAGUGGCACCGAGUCGGU | |
| | GCUsmUsmUsmU | |
| gRNA1401 | mCsmAsmCsUUGCCAGCAACAAAGUAGUUUUAGAGCUAGAAAUAGCAAGU | 705 |
| | UAAAAUAAGGCUAGUCCGUUAUCAACUUGAAAAAGUGGCACCGAGUCGGU | |
| | GCUsmUsmUsmU | |
| gRNA1402 | mGsmCsmUsCAAUAUGCCCCAGGCUAGUUUUAGAGCUAGAAAUAGCAAGU | 706 |
| | UAAAAUAAGGCUAGUCCGUUAUCAACUUGAAAAAGUGGCACCGAGUCGGU | |
| | GCUsmUsmUsmU | |
| gRNA1403 | mCsmUsmUsUCAAGUGCUGCCCUGGCGUUUUAGAGCUAGAAAUAGCAAGU | 707 |
| | UAAAAUAAGGCUAGUCCGUUAUCAACUUGAAAAAGUGGCACCGAGUCGGU | |
| | GCUsmUsmUsmU | |
| gRNA1404 | mCsmCsmUsUCAGCCAGGGCAGCACUGUUUUAGAGCUAGAAAUAGCAAGU | 708 |
| | UAAAAUAAGGCUAGUCCGUUAUCAACUUGAAAAAGUGGCACCGAGUCGGU | |
| | GCUsmUsmUsmU | |
| gRNA1405 | mCsmAsmGsCCAGGGCAGCACUUGAAGUUUUAGAGCUAGAAAUAGCAAGU | 709 |
| | UAAAAUAAGGCUAGUCCGUUAUCAACUUGAAAAAGUGGCACCGAGUCGGU | |
| | GCUsmUsmUsmU | |
| gRNA1406 | mGsmCsmCsUUUCUGUAGGGGAGGUGGUUUUAGAGCUAGAAAUAGCAAGU | 710 |
| | UAAAAUAAGGCUAGUCCGUUAUCAACUUGAAAAAGUGGCACCGAGUCGGU | |
| | GCUsmUsmUsmU | |
| gRNA1407 | mAsmAsmUsGGGCCUAUAAAGGACAAGUUUUAGAGCUAGAAAUAGCAAGU | 711 |
| | UAAAAUAAGGCUAGUCCGUUAUCAACUUGAAAAAGUGGCACCGAGUCGGU | |
| | GCUsmUsmUsmU | |
| gRNA1408 | mUsmGsmGsGCCUAUAAAGGACAAGGGUUUUAGAGCUAGAAAUAGCAAGU | 712 |
| | UAAAAUAAGGCUAGUCCGUUAUCAACUUGAAAAAGUGGCACCGAGUCGGU | |
| | GCUsmUsmUsmU | |
| gRNA1409 | mGsmUsmGsUUCCCAAACCAUGCCACGUUUUAGAGCUAGAAAUAGCAAGU | 713 |
| | UAAAAUAAGGCUAGUCCGUUAUCAACUUGAAAAAGUGGCACCGAGUCGGU | |
| | GCUsmUsmUsmU | |
| gRNA1410 | mGsmUsmUsCCCAAACCAUGCCACAGGUUUUAGAGCUAGAAAUAGCAAGU | 714 |
| | UAAAAUAAGGCUAGUCCGUUAUCAACUUGAAAAAGUGGCACCGAGUCGGU | |
| | GCUsmUsmUsmU | |
| gRNA1411 | mGsmCsmGsGUUCCAGCCUUCAGGAGGUUUUAGAGCUAGAAAUAGCAAGU | 715 |
| | UAAAAUAAGGCUAGUCCGUUAUCAACUUGAAAAAGUGGCACCGAGUCGGU | |
| | GCUsmUsmUsmU | |
| gRNA1412 | mUsmUsmGsUGGUCUGUGGAGAGGGAGUUUUAGAGCUAGAAAUAGCAAGU | 716 |
| | UAAAAUAAGGCUAGUCCGUUAUCAACUUGAAAAAGUGGCACCGAGUCGGU | |
| | GCUsmUsmUsmU | |
| gRNA1413 | mGsmAsmGsUCUGGGAGAGUCAGAAAGUUUUAGAGCUAGAAAUAGCAAGU | 717 |
| | UAAAAUAAGGCUAGUCCGUUAUCAACUUGAAAAAGUGGCACCGAGUCGGU | |
| | GCUsmUsmUsmU | |
| gRNA1414 | mCsmGsmCsUCCAAGAGCCACCUUCCGUUUUAGAGCUAGAAAUAGCAAGU | 718 |
| | UAAAAUAAGGCUAGUCCGUUAUCAACUUGAAAAAGUGGCACCGAGUCGGU | |
| | GCUsmUsmUsmU | |
| gRNA1415 | mUsmAsmCsCCCGCUGCAGAGGUAUGGUUUUAGAGCUAGAAAUAGCAAGU | 719 |
| | UAAAAUAAGGCUAGUCCGUUAUCAACUUGAAAAAGUGGCACCGAGUCGGU | |
| | GCUsmUsmUsmU | |
| gRNA1416 | mGsmGsmGsCCAGUAUUCCCCGUUCUGUUUUAGAGCUAGAAAUAGCAAGU | 720 |
| | UAAAAUAAGGCUAGUCCGUUAUCAACUUGAAAAAGUGGCACCGAGUCGGU | |
| | GCUsmUsmUsmU | |
| gRNA1417 | mUsmGsmGsAUUGCUUUGAGAAGGGGGUUUUAGAGCUAGAAAUAGCAAGU | 721 |
| | UAAAAUAAGGCUAGUCCGUUAUCAACUUGAAAAAGUGGCACCGAGUCGGU | |
| | GCUsmUsmUsmU | |
| gRNA1418 | mAsmGsmGsGUGCUCCAGGACCCCGUGUUUUAGAGCUAGAAAUAGCAAGU | 722 |
| | UAAAAUAAGGCUAGUCCGUUAUCAACUUGAAAAAGUGGCACCGAGUCGGU | |
| | GCUsmUsmUsmU | |
| gRNA1419 | mCsmAsmCsCUGAAGAGAAAGGCUGAGUUUUAGAGCUAGAAAUAGCAAGU | 723 |
| | UAAAAUAAGGCUAGUCCGUUAUCAACUUGAAAAAGUGGCACCGAGUCGGU | |
| | GCUsmUsmUsmU | |

⁴“mN” indicates a 2′-OMe modification of the nucleotide “N”, and “Ns” indicates that the nucleotide “N” is linked to the following (i.e., 3′) nucleotide by a phosphorothioate (PS).

TABLE 1E
Representative sequences for guide polynucleotides for use in guiding a base
editor to alter a complement_factor B_splice site or introduce a new stop
codon into a complement_factor B polynucleotide using base editing.⁵

| Guidepolynucleotide name | Guidepolynucleotide sequence | SEQ ID NO |
|---|---|---|
| GuideDesign10_CBE_NGA_ | mCsmUsmUsCUCACCUCCGUUGUCACGUUUUAGAGCUAGA | 724 |
| 20nt_4-9_006- | AAUAGCAAGUUAAAAUAAGGCUAGUCCGUUAUCAACUUGA | |
| 31947177_spCas9 | AAAAGUGGCACCGAGUCGGUGCUsmUsmUsmU | |
| GuideDesign10_CBE_NGA_ | mCsmUsmGsACCAUCAGUCAUGAGGAGUUUUAGAGCUAGA | 725 |
| 20nt_4-9_006- | AAUAGCAAGUUAAAAUAAGGCUAGUCCGUUAUCAACUUGA | |
| 31948943_spCas9 | AAAAGUGGCACCGAGUCGGUGCUsmUsmUsmU | |
| GuideDesign10_CBE_NGA_ | mCsmUsmCsCCUACCGAUCAUUUGGUGUUUUAGAGCUAGA | 726 |
| 20nt_4-9_006- | AAUAGCAAGUUAAAAUAAGGCUAGUCCGUUAUCAACUUGA | |
| 31949542_spCas9 | AAAAGUGGCACCGAGUCGGUGCUsmUsmUsmU | |
| GuideDesign10_CBE_NGA_ | mUsmUsmAsCCUACGCUGACCUUGAUGUUUUAGAGCUAGA | 727 |
| 20nt_4-9_006- | AAUAGCAAGUUAAAAUAAGGCUAGUCCGUUAUCAACUUGA | |
| 31950384_spCas9 | AAAAGUGGCACCGAGUCGGUGCUsmUsmUsmU | |
| GuideDesign10_CBE_NGA_ | mAsmUsmCsCUUACCUACGCUGACCUGUUUUAGAGCUAGA | 728 |
| 20nt_4-9_006- | AAUAGCAAGUUAAAAUAAGGCUAGUCCGUUAUCAACUUGA | |
| 31950388_spCas9 | AAAAGUGGCACCGAGUCGGUGCUsmUsmUsmU | |
| GuideDesign10_CBE_NGA_ | mUsmCsmUsCACCUUAUCCCCAUUCUGUUUUAGAGCUAGA | 729 |
| 20nt_4-9_006- | AAUAGCAAGUUAAAAUAAGGCUAGUCCGUUAUCAACUUGA | |
| 31951227_spCas9 | AAAAGUGGCACCGAGUCGGUGCUsmUsmUsmU | |
| GuideDesign10_CBE_NGC_ | mUsmCsmGsCUUACCUCCAGACAAGAGUUUUAGAGCUAGA | 730 |
| 20nt_4-9_009- | AAUAGCAAGUUAAAAUAAGGCUAGUCCGUUAUCAACUUGA | |
| 31946270_spCas9 | AAAAGUGGCACCGAGUCGGUGCUsmUsmUsmU | |
| GuideDesign10_CBE_NGC_ | mUsmCsmAsCCUCCGUUGUCACAGAUGUUUUAGAGCUAGA | 731 |
| 20nt_4-9_009- | AAUAGCAAGUUAAAAUAAGGCUAGUCCGUUAUCAACUUGA | |
| 31947173_spCas9 | AAAAGUGGCACCGAGUCGGUGCUsmUsmUsmU | |
| GuideDesign10_CBE_NGC_ | mCsmUsmUsACAAUGACUGAGAUCUUGUUUUAGAGCUAGA | 732 |
| 20nt_4-9_009- | AAUAGCAAGUUAAAAUAAGGCUAGUCCGUUAUCAACUUGA | |
| 31950129_spCas9 | AAAAGUGGCACCGAGUCGGUGCUsmUsmUsmU | |
| GuideDesign10_CBE_NGC_ | mAsmAsmCsCUGCAGAGGCAGUGGGGGUUUUAGAGCUAGA | 733 |
| 20nt_4-9_009_- | AAUAGCAAGUUAAAAUAAGGCUAGUCCGUUAUCAACUUGA | |
| 31951854_spCas9 | AAAAGUGGCACCGAGUCGGUGCUsmUsmUsmU | |
| GuideDesign10_CBE_NGG_ | mCsmUsmCsGCUUACCUCCAGACAAGGUUUUAGAGCUAGA | 734 |
| 20nt_4-9_003- | AAUAGCAAGUUAAAAUAAGGCUAGUCCGUUAUCAACUUGA | |
| 31946271_spCas9 | AAAAGUGGCACCGAGUCGGUGCUsmUsmUsmU | |
| GuideDesign10_CBE_NGG_ | mCsmCsmUsUCUGACCAUCAGUCAUGGUUUUAGAGCUAGA | 735 |
| 20nt_4-9_003- | AAUAGCAAGUUAAAAUAAGGCUAGUCCGUUAUCAACUUGA | |
| 31948947_spCas9 | AAAAGUGGCACCGAGUCGGUGCUsmUsmUsmU | |
| GuideDesign10_CBE_NGG_ | mUsmCsmAsCCCAGAUAAUCCUCCCUGUUUUAGAGCUAGA | 736 |
| 20nt_4-9_003- | AAUAGCAAGUUAAAAUAAGGCUAGUCCGUUAUCAACUUGA | |
| 31949325_spCas9 | AAAAGUGGCACCGAGUCGGUGCUsmUsmUsmU | |
| GuideDesign10_CBE_NGG_ | mGsmCsmUSUACAAUGACUGAGAUCUGUUUUAGAGCUAGA | 737 |
| 20nt_4-9_003- | AAUAGCAAGUUAAAAUAAGGCUAGUCCGUUAUCAACUUGA | |
| 31950130_spCas9 | AAAAGUGGCACCGAGUCGGUGCUsmUsmUsmU | |
| GuideDesign10_CBE_NGG_ | mCsmUsmUsACUUUGUUGCUGGCAAGGUUUUAGAGCUAGA | 738 |
| 20nt_4-9_003- | AAUAGCAAGUUAAAAUAAGGCUAGUCCGUUAUCAACUUGA | |
| 31950926_spCas9 | AAAAGUGGCACCGAGUCGGUGCUsmUsmUsmU | |
| GuideDesign10_CBE_NGG_ | mUsmCsmUsCACCUCUGCAAGUAUUGGUUUUAGAGCUAGA | 739 |
| 20nt_4-9_003- | AAUAGCAAGUUAAAAUAAGGCUAGUCCGUUAUCAACUUGA | |
| 31951456_spCas9 | AAAAGUGGCACCGAGUCGGUGCUsmUsmUsmU | |
| GuideDesign3_ABE_NGA_ | mCsmCsmCsUCGCUUACCUCCAGACAGUUUUAGAGCUAGA | 740 |
| 20nt_3-12_019- | AAUAGCAAGUUAAAAUAAGGCUAGUCCGUUAUCAACUUGA | |
| 31946273_spCas9 | AAAAGUGGCACCGAGUCGGUGCUsmUsmUsmU | |
| GuideDesign3_ABE_NGA_ | mCsmUsmUsCUCACCUCCGUUGUCACGUUUUAGAGCUAGA | 741 |
| 20nt_3-12_019- | AAUAGCAAGUUAAAAUAAGGCUAGUCCGUUAUCAACUUGA | |
| 31947177_spCas9 | AAAAGUGGCACCGAGUCGGUGCUsmUsmUsmU | |
| GuideDesign3_ABE_NGA_ | mGsmGsmUsCAAAGGUCACCUUGGCAGUUUUAGAGCUAGA | 742 |
| 20nt_3-12_019_- | AAUAGCAAGUUAAAAUAAGGCUAGUCCGUUAUCAACUUGA | |
| 31947511_spCas9 | AAAAGUGGCACCGAGUCGGUGCUsmUsmUsmU | |
| GuideDesign3_ABE_NGA_ | mUsmUsmCsCACCUUCUCAAUUAAGUGUUUUAGAGCUAGA | 743 |
| 20nt_3-12_019- | AAUAGCAAGUUAAAAUAAGGCUAGUCCGUUAUCAACUUGA | |
| 31948064_spCas9 | AAAAGUGGCACCGAGUCGGUGCUsmUsmUsmU | |
| GuideDesign3_ABE_NGA_ | mUsmAsmAsCCUCUGACCUUCAUAAUGUUUUAGAGCUAGA | 744 |
| 20nt_3-12_019- | AAUAGCAAGUUAAAAUAAGGCUAGUCCGUUAUCAACUUGA | |
| 31948500_spCas9 | AAAAGUGGCACCGAGUCGGUGCUsmUsmUsmU | |
| GuideDesign3_ABE_NGA_ | mCsmUsmGsACCAUCAGUCAUGAGGAGUUUUAGAGCUAGA | 745 |
| 20nt_3-12_019- | AAUAGCAAGUUAAAAUAAGGCUAGUCCGUUAUCAACUUGA | |
| 31948943_spCas9 | AAAAGUGGCACCGAGUCGGUGCUsmUsmUsmU | |
| GuideDesign3_ABE_NGA_ | mCsmUsmUsCUGACCAUCAGUCAUGAGUUUUAGAGCUAGA | 746 |
| 20nt_3-12_019- | AAUAGCAAGUUAAAAUAAGGCUAGUCCGUUAUCAACUUGA | |
| 31948946_spCas9 | AAAAGUGGCACCGAGUCGGUGCUsmUsmUsmU | |
| GuideDesign3_ABE_NGA_ | mUsmCsmCsCUUCUGACCAUCAGUCAGUUUUAGAGCUAGA | 747 |
| 20nt_3-12_019- | AAUAGCAAGUUAAAAUAAGGCUAGUCCGUUAUCAACUUGA | |
| 31948949_spCas9 | AAAAGUGGCACCGAGUCGGUGCUsmUsmUsmU | |
| GuideDesign3_ABE_NGA_ | mCsmUsmCsCCUACCGAUCAUUUGGUGUUUUAGAGCUAGA | 748 |
| 20nt_3-12_019- | AAUAGCAAGUUAAAAUAAGGCUAGUCCGUUAUCAACUUGA | |
| 31949542_spCas9 | AAAAGUGGCACCGAGUCGGUGCUsmUsmUsmU | |
| GuideDesign3_ABE_NGA_ | mUsmUsmCsUGUGCUUACAAUGACUGGUUUUAGAGCUAGA | 749 |
| 20nt_3-12_019- | AAUAGCAAGUUAAAAUAAGGCUAGUCCGUUAUCAACUUGA | |
| 31950136_spCas9 | AAAAGUGGCACCGAGUCGGUGCUsmUsmUsmU | |
| GuideDesign3_ABE_NGA_ | mUsmUsmAsCCUACGCUGACCUUGAUGUUUUAGAGCUAGA | 750 |
| 20nt_3-12_019- | AAUAGCAAGUUAAAAUAAGGCUAGUCCGUUAUCAACUUGA | |
| 31950384_spCas9 | AAAAGUGGCACCGAGUCGGUGCUsmUsmUsmU | |
| GuideDesign3_ABE_NGA_ | mAsmUsmCsCUUACCUACGCUGACCUGUUUUAGAGCUAGA | 751 |
| 20nt_3-12_019_- | AAUAGCAAGUUAAAAUAAGGCUAGUCCGUUAUCAACUUGA | |
| 31950388_spCas9 | AAAAGUGGCACCGAGUCGGUGCUsmUsmUsmU | |
| GuideDesign3_ABE_NGA_ | mUsmCsmUsCACCUUAUCCCCAUUCUGUUUUAGAGCUAGA | 752 |
| 20nt_3-12_019- | AAUAGCAAGUUAAAAUAAGGCUAGUCCGUUAUCAACUUGA | |
| 31951227_spCas9 | AAAAGUGGCACCGAGUCGGUGCUsmUsmUsmU | |
| GuideDesign3_ABE_NGA_ | mGsmAsmGsGACUCACUUGAAUGAAAGUUUUAGAGCUAGA | 753 |
| 20nt_3-12_019- | AAUAGCAAGUUAAAAUAAGGCUAGUCCGUUAUCAACUUGA | |
| 31951591_spCas9 | AAAAGUGGCACCGAGUCGGUGCUsmUsmUsmU | |
| GuideDesign3_ABE_NGA_ | mUsmCsmAsAAGCAAUCCACUGUCCAGUUUUAGAGCUAGA | 754 |
| 20nt_3- | AAUAGCAAGUUAAAAUAAGGCUAGUCCGUUAUCAACUUGA | |
| 12_019_+_31947000_spCas9 | AAAAGUGGCACCGAGUCGGUGCUsmUsmUsmU | |
| GuideDesign3_ABE_NGA_ | mCsmUsmCsCCAGACUCCUUCAUGUAGUUUUAGAGCUAGA | 755 |
| 20nt_3- | AAUAGCAAGUUAAAAUAAGGCUAGUCCGUUAUCAACUUGA | |
| 12_019_+_31947734_spCas9 | AAAAGUGGCACCGAGUCGGUGCUsmUsmUsmU | |
| GuideDesign3_ABE_NGA_ | mCsmUsmGsGCACCCAGGGGAACAACGUUUUAGAGCUAGA | 756 |
| 20nt_3- | AAUAGCAAGUUAAAAUAAGGCUAGUCCGUUAUCAACUUGA | |
| 12_019_+_31947933_spCas9 | AAAAGUGGCACCGAGUCGGUGCUsmUsmUsmU | |
| GuideDesign3_ABE_NGA_ | mCsmCsmCsAGGGGAACAACAGAAGCGUUUUAGAGCUAGA | 757 |
| 20nt_3- | AAUAGCAAGUUAAAAUAAGGCUAGUCCGUUAUCAACUUGA | |
| 12_019_+_31947939_spCas9 | AAAAGUGGCACCGAGUCGGUGCUsmUsmUsmU | |
| GuideDesign3_ABE_NGA_ | mAsmCsmAsGGUGGCAAGUUAUGGUGGUUUUAGAGCUAGA | 758 |
| 20nt_3- | AAUAGCAAGUUAAAAUAAGGCUAGUCCGUUAUCAACUUGA | |
| 12_019_+_31948369_spCas9 | AAAAGUGGCACCGAGUCGGUGCUsmUsmUsmU | |
| GuideDesign3_ABE_NGA_ | mCsmCsmUsCUCCACAGACCACAAGUGUUUUAGAGCUAGA | 759 |
| 20nt_3- | AAUAGCAAGUUAAAAUAAGGCUAGUCCGUUAUCAACUUGA | |
| 12_019_+_31948818_spCas9 | AAAAGUGGCACCGAGUCGGUGCUsmUsmUsmU | |
| GuideDesign3_ABE_NGA_ | mGsmCsmCsAGAUGAAAGCCAGUCUCGUUUUAGAGCUAGA | 760 |
| 20nt_3- | AAUAGCAAGUUAAAAUAAGGCUAGUCCGUUAUCAACUUGA | |
| 12_019_+_31950044_spCas9 | AAAAGUGGCACCGAGUCGGUGCUsmUsmUsmU | |
| GuideDesign3_ABE_NGA_ | mGsmUsmCsCCCAGCGCCCUUCAAAGGUUUUAGAGCUAGA | 761 |
| 20nt_3- | AAUAGCAAGUUAAAAUAAGGCUAGUCCGUUAUCAACUUGA | |
| 12_019_+_31950277_spCas9 | AAAAGUGGCACCGAGUCGGUGCUsmUsmUsmU | |
| GuideDesign3_ABE_NGA_ | mAsmCsmUSUCAGGAGGGGAGAAGCGGUUUUAGAGCUAGA | 762 |
| 20nt_3- | AAUAGCAAGUUAAAAUAAGGCUAGUCCGUUAUCAACUUGA | |
| 12_019_+_31950611_spCas9 | AAAAGUGGCACCGAGUCGGUGCUsmUsmUsmU | |
| GuideDesign3_ABE_NGA_ | mCsmCsmCsCUACAGAAAGGCAGCUGGUUUUAGAGCUAGA | 763 |
| 20nt_3- | AAUAGCAAGUUAAAAUAAGGCUAGUCCGUUAUCAACUUGA | |
| 12_019_+_31951331_spCas9 | AAAAGUGGCACCGAGUCGGUGCUsmUsmUsmU | |
| GuideDesign3_ABE_NGA_ | mCsmCsmUsACAGAAAGGCAGCUGUGGUUUUAGAGCUAGA | 764 |
| 20nt_3- | AAUAGCAAGUUAAAAUAAGGCUAGUCCGUUAUCAACUUGA | |
| 12_019_+_31951333_spCas9 | AAAAGUGGCACCGAGUCGGUGCUsmUsmUsmU | |
| GuideDesign3_ABE_NGA_ | mUsmAsmCsAGAAAGGCAGCUGUGAGGUUUUAGAGCUAGA | 765 |
| 20nt_3- | AAUAGCAAGUUAAAAUAAGGCUAGUCCGUUAUCAACUUGA | |
| 12_019_+_31951335_spCas9 | AAAAGUGGCACCGAGUCGGUGCUsmUsmUsmU | |
| GuideDesign3_ABE_NGA_ | mUsmUsmCsCACCUUCUCAAUUAAGUGUUUUAGAGCUAGA | 766 |
| 20nt_3-9_005- | AAUAGCAAGUUAAAAUAAGGCUAGUCCGUUAUCAACUUGA | |
| 31948064_spCas9 | AAAAGUGGCACCGAGUCGGUGCUsmUsmUsmU | |
| GuideDesign3_ABE_NGA_ | mCsmUsmUsCUGACCAUCAGUCAUGAGUUUUAGAGCUAGA | 767 |
| 20nt_3-9_005- | AAUAGCAAGUUAAAAUAAGGCUAGUCCGUUAUCAACUUGA | |
| 31948946_spCas9 | AAAAGUGGCACCGAGUCGGUGCUsmUsmUsmU | |
| GuideDesign3_ABE_NGA_ | mAsmCsmUsUCAGGAGGGGAGAAGCGGUUUUAGAGCUAGA | 768 |
| 20nt_3- | AAUAGCAAGUUAAAAUAAGGCUAGUCCGUUAUCAACUUGA | |
| 9_005_+_31950611_spCas9 | AAAAGUGGCACCGAGUCGGUGCUsmUsmUsmU | |
| GuideDesign3_ABE_NGA_ | mCsmCsmCsCUACAGAAAGGCAGCUGGUUUUAGAGCUAGA | 769 |
| 20nt_3- | AAUAGCAAGUUAAAAUAAGGCUAGUCCGUUAUCAACUUGA | |
| 9_005_+_31951331_spCas9 | AAAAGUGGCACCGAGUCGGUGCUsmUsmUsmU | |
| GuideDesign3_ABE_NGA_ | mCsmCsmUsACAGAAAGGCAGCUGUGGUUUUAGAGCUAGA | 770 |
| 20nt_3- | AAUAGCAAGUUAAAAUAAGGCUAGUCCGUUAUCAACUUGA | |
| 9_005_+_31951333_spCas9 | AAAAGUGGCACCGAGUCGGUGCUsmUsmUsmU | |
| GuideDesign3_ABE_NGC_ | mUsmCsmGsCUUACCUCCAGACAAGAGUUUUAGAGCUAGA | 771 |
| 20nt_3-12_020- | AAUAGCAAGUUAAAAUAAGGCUAGUCCGUUAUCAACUUGA | |
| 31946270_spCas9 | AAAAGUGGCACCGAGUCGGUGCUsmUsmUsmU | |
| GuideDesign3_ABE_NGC_ | mGsmCsmCsCUCAAACCUCUGCACUCGUUUUAGAGCUAGA | 772 |
| 20nt_3-12_020- | AAUAGCAAGUUAAAAUAAGGCUAGUCCGUUAUCAACUUGA | |
| 31946593_spCas9 | AAAAGUGGCACCGAGUCGGUGCUsmUsmUsmU | |
| GuideDesign3_ABE_NGC_ | mUsmCsmAsCCUCCGUUGUCACAGAUGUUUUAGAGCUAGA | 773 |
| 20nt_3-12_020- | AAUAGCAAGUUAAAAUAAGGCUAGUCCGUUAUCAACUUGA | |
| 31947173_spCas9 | AAAAGUGGCACCGAGUCGGUGCUsmUsmUsmU | |
| GuideDesign3_ABE_NGC_ | mAsmAsmAsGGUCACCUUGGCAGGAAGUUUUAGAGCUAGA | 774 |
| 20nt_3-12_020- | AAUAGCAAGUUAAAAUAAGGCUAGUCCGUUAUCAACUUGA | |
| 31947507_spCas9 | AAAAGUGGCACCGAGUCGGUGCUsmUsmUsmU | |
| GuideDesign3_ABE_NGC_ | mCsmUsmGsUCUUCAAACCUGGGCCGGUUUUAGAGCUAGA | 775 |
| 20nt_3-12_020- | AAUAGCAAGUUAAAAUAAGGCUAGUCCGUUAUCAACUUGA | |
| 31947832_spCas9 | AAAAGUGGCACCGAGUCGGUGCUsmUsmUsmU | |
| GuideDesign3_ABE_NGC_ | mCsmUsmUsACAAUGACUGAGAUCUUGUUUUAGAGCUAGA | 776 |
| 20nt_3-12_020- | AAUAGCAAGUUAAAAUAAGGCUAGUCCGUUAUCAACUUGA | |
| 31950129_spCas9 | AAAAGUGGCACCGAGUCGGUGCUsmUsmUsmU | |
| GuideDesign3_ABE_NGC_ | mAsmCsmGsCUCUCACCUGAUAGUCUGUUUUAGAGCUAGA | 777 |
| 20nt_3-12_020_- | AAUAGCAAGUUAAAAUAAGGCUAGUCCGUUAUCAACUUGA | |
| 31950759_spCas9 | AAAAGUGGCACCGAGUCGGUGCUsmUsmUsmU | |
| GuideDesign3_ABE_NGC_ | mGsmUsmAsUGUCUUACUUUGUUGCUGUUUUAGAGCUAGA | 778 |
| 20nt_3-12_020- | AAUAGCAAGUUAAAAUAAGGCUAGUCCGUUAUCAACUUGA | |
| 31950932_spCas9 | AAAAGUGGCACCGAGUCGGUGCUsmUsmUsmU | |
| GuideDesign3_ABE_NGC_ | mAsmUsmAsCCUCUGCAGCGGGGUACGUUUUAGAGCUAGA | 779 |
| 20nt_3- | AAUAGCAAGUUAAAAUAAGGCUAGUCCGUUAUCAACUUGA | |
| 12_020_+_31947335_spCas9 | AAAAGUGGCACCGAGUCGGUGCUsmUsmUsmU | |
| GuideDesign3_ABE_NGC_ | mGsmCsmAsCCCAGGGGAACAACAGAGUUUUAGAGCUAGA | 780 |
| 20nt_3- | AAUAGCAAGUUAAAAUAAGGCUAGUCCGUUAUCAACUUGA | |
| 12_020_+_31947936_spCas9 | AAAAGUGGCACCGAGUCGGUGCUsmUsmUsmU | |
| GuideDesign3_ABE_NGC_ | mCsmUsmGsCAGGAUUGCACAACAUGGUUUUAGAGCUAGA | 781 |
| 20nt_3- | AAUAGCAAGUUAAAAUAAGGCUAGUCCGUUAUCAACUUGA | |
| 12_020_+_31949236_spCas9 | AAAAGUGGCACCGAGUCGGUGCUsmUsmUsmU | |
| GuideDesign3_ABE_NGC_ | mUsmCsmCsUACUUCAGGAGGGGAGAGUUUUAGAGCUAGA | 782 |
| 20nt_3- | AAUAGCAAGUUAAAAUAAGGCUAGUCCGUUAUCAACUUGA | |
| 12_020_+_31950607_spCas9 | AAAAGUGGCACCGAGUCGGUGCUsmUsmUsmU | |
| GuideDesign3_ABE_NGC_ | mAsmUsmAsGGCCCAUUUGUCUCCCCGUUUUAGAGCUAGA | 783 |
| 20nt_3- | AAUAGCAAGUUAAAAUAAGGCUAGUCCGUUAUCAACUUGA | |
| 12_020_+_31950863_spCas9 | AAAAGUGGCACCGAGUCGGUGCUsmUsmUsmU | |
| GuideDesign3_ABE_NGC_ | mUsmAsmUsUCGUCCAGAGGAAGAGCGUUUUAGAGCUAGA | 784 |
| 20nt_3- | AAUAGCAAGUUAAAAUAAGGCUAGUCCGUUAUCAACUUGA | |
| 12_020_+_31951132_spCas9 | AAAAGUGGCACCGAGUCGGUGCUsmUsmUsmU | |
| GuideDesign3_ABE_NGC_ | mCsmCsmAsGAGGAAGAGCUGCUCCCGUUUUAGAGCUAGA | 785 |
| 20nt_3- | AAUAGCAAGUUAAAAUAAGGCUAGUCCGUUAUCAACUUGA | |
| 12_020_+_31951139_spCas9 | AAAAGUGGCACCGAGUCGGUGCUsmUsmUsmU | |
| GuideDesign3_ABE_NGC_ | mCsmUsmCSUCCUUGCAGGUGAUUCUGUUUUAGAGCUAGA | 786 |
| 20nt_3- | AAUAGCAAGUUAAAAUAAGGCUAGUCCGUUAUCAACUUGA | |
| 12_020_+_31951542_spCas9 | AAAAGUGGCACCGAGUCGGUGCUsmUsmUsmU | |
| GuideDesign3_ABE_NGC_ | mUsmCsmCsUUGCAGGUGAUUCUGGCGUUUUAGAGCUAGA | 787 |
| 20nt_3- | AAUAGCAAGUUAAAAUAAGGCUAGUCCGUUAUCAACUUGA | |
| 12_020_+_31951545_spCas9 | AAAAGUGGCACCGAGUCGGUGCUsmUsmUsmU | |
| GuideDesign3_ABE_NGC_ | mCsmUsmCsUGCAGGUUGGUGUAAUCGUUUUAGAGCUAGA | 788 |
| 20nt_3- | AAUAGCAAGUUAAAAUAAGGCUAGUCCGUUAUCAACUUGA | |
| 12_020_+_31951866_spCas9 | AAAAGUGGCACCGAGUCGGUGCUsmUsmUsmU | |
| GuideDesign3_ABE_NGC_ | mGsmCsmCsCUCAAACCUCUGCACUCGUUUUAGAGCUAGA | 789 |
| 20nt_3-9_008- | AAUAGCAAGUUAAAAUAAGGCUAGUCCGUUAUCAACUUGA | |
| 31946593_spCas9 | AAAAGUGGCACCGAGUCGGUGCUsmUsmUsmU | |
| GuideDesign3_ABE_NGC_ | mAsmAsmAsGGUCACCUUGGCAGGAAGUUUUAGAGCUAGA | 790 |
| 20nt_3-9_008- | AAUAGCAAGUUAAAAUAAGGCUAGUCCGUUAUCAACUUGA | |
| 31947507_spCas9 | AAAAGUGGCACCGAGUCGGUGCUsmUsmUsmU | |
| GuideDesign3_ABE_NGC_ | mGsmCsmAsCCCAGGGGAACAACAGAGUUUUAGAGCUAGA | 791 |
| 20nt_3- | AAUAGCAAGUUAAAAUAAGGCUAGUCCGUUAUCAACUUGA | |
| 9_008_+_31947936_spCas9 | AAAAGUGGCACCGAGUCGGUGCUsmUsmUsmU | |
| GuideDesign3_ABE_NGC_ | mCsmCsmAsGAGGAAGAGCUGCUCCCGUUUUAGAGCUAGA | 792 |
| 20nt_3- | AAUAGCAAGUUAAAAUAAGGCUAGUCCGUUAUCAACUUGA | |
| 9_008_+_31951139_spCas9 | AAAAGUGGCACCGAGUCGGUGCUsmUsmUsmU | |
| GuideDesign3_ABE_NGC_ | mUsmCsmCsUUGCAGGUGAUUCUGGCGUUUUAGAGCUAGA | 793 |
| 20nt_3- | AAUAGCAAGUUAAAAUAAGGCUAGUCCGUUAUCAACUUGA | |
| 9_008_+_31951545_spCas9 | AAAAGUGGCACCGAGUCGGUGCUsmUsmUsmU | |
| GuideDesign3_ABE_NGG_ | mCsmUsmCsGCUUACCUCCAGACAAGGUUUUAGAGCUAGA | 794 |
| 20nt_3-12_018- | AAUAGCAAGUUAAAAUAAGGCUAGUCCGUUAUCAACUUGA | |
| 31946271_spCas9 | AAAAGUGGCACCGAGUCGGUGCUsmUsmUsmU | |
| GuideDesign3_ABE_NGG_ | mCsmAsmAsAGGUCACCUUGGCAGGAGUUUUAGAGCUAGA | 795 |
| 20nt_3-12_018- | AAUAGCAAGUUAAAAUAAGGCUAGUCCGUUAUCAACUUGA | |
| 31947508_spCas9 | AAAAGUGGCACCGAGUCGGUGCUsmUsmUsmU | |
| GuideDesign3_ABE_NGG_ | mCsmCsmUsUCUGACCAUCAGUCAUGGUUUUAGAGCUAGA | 796 |
| 20nt_3-12_018- | AAUAGCAAGUUAAAAUAAGGCUAGUCCGUUAUCAACUUGA | |
| 31948947_spCas9 | AAAAGUGGCACCGAGUCGGUGCUsmUsmUsmU | |
| GuideDesign3_ABE_NGG_ | mUsmCsmAsCCCAGAUAAUCCUCCCUGUUUUAGAGCUAGA | 797 |
| 20nt_3-12_018- | AAUAGCAAGUUAAAAUAAGGCUAGUCCGUUAUCAACUUGA | |
| 31949325_spCas9 | AAAAGUGGCACCGAGUCGGUGCUsmUsmUsmU | |
| GuideDesign3_ABE_NGG_ | mGsmUsmAsUCUCCCUACCGAUCAUUGUUUUAGAGCUAGA | 798 |
| 20nt_3-12_018- | AAUAGCAAGUUAAAAUAAGGCUAGUCCGUUAUCAACUUGA | |
| 31949546_spCas9 | AAAAGUGGCACCGAGUCGGUGCUsmUsmUsmU | |
| GuideDesign3_ABE_NGG_ | mGsmCsmUSUACAAUGACUGAGAUCUGUUUUAGAGCUAGA | 799 |
| 20nt_3-12_018- | AAUAGCAAGUUAAAAUAAGGCUAGUCCGUUAUCAACUUGA | |
| 31950130_spCas9 | AAAAGUGGCACCGAGUCGGUGCUsmUsmUsmU | |
| GuideDesign3_ABE_NGG_ | mGsmAsmCsGCUCUCACCUGAUAGUCGUUUUAGAGCUAGA | 800 |
| 20nt_3-12_018- | AAUAGCAAGUUAAAAUAAGGCUAGUCCGUUAUCAACUUGA | |
| 31950760_spCas9 | AAAAGUGGCACCGAGUCGGUGCUsmUsmUsmU | |
| GuideDesign3_ABE_NGG_ | mCsmUsmUsACUUUGUUGCUGGCAAGGUUUUAGAGCUAGA | 801 |
| 20nt_3-12_018- | AAUAGCAAGUUAAAAUAAGGCUAGUCCGUUAUCAACUUGA | |
| 31950926_spCas9 | AAAAGUGGCACCGAGUCGGUGCUsmUsmUsmU | |
| GuideDesign3_ABE_NGG_ | mAsmGsmUsAUGUCUUACUUUGUUGCGUUUUAGAGCUAGA | 802 |
| 20nt_3-12_018- | AAUAGCAAGUUAAAAUAAGGCUAGUCCGUUAUCAACUUGA | |
| 31950933_spCas9 | AAAAGUGGCACCGAGUCGGUGCUsmUsmUsmU | |
| GuideDesign3_ABE_NGG_ | mUsmCsmUsCACCUCUGCAAGUAUUGGUUUUAGAGCUAGA | 803 |
| 20nt_3-12_018- | AAUAGCAAGUUAAAAUAAGGCUAGUCCGUUAUCAACUUGA | |
| 31951456_spCas9 | AAAAGUGGCACCGAGUCGGUGCUsmUsmUsmU | |
| GuideDesign3_ABE_NGG_ | mCsmUsmCsUCACCUCUGCAAGUAUUGUUUUAGAGCUAGA | 804 |
| 20nt_3-12_018- | AAUAGCAAGUUAAAAUAAGGCUAGUCCGUUAUCAACUUGA | |
| 31951457_spCas9 | AAAAGUGGCACCGAGUCGGUGCUsmUsmUsmU | |
| GuideDesign3_ABE_NGG_ | mUsmCsmUsCUCACCUCUGCAAGUAUGUUUUAGAGCUAGA | 805 |
| 20nt_3-12_018- | AAUAGCAAGUUAAAAUAAGGCUAGUCCGUUAUCAACUUGA | |
| 31951458_spCas9 | AAAAGUGGCACCGAGUCGGUGCUsmUsmUsmU | |
| GuideDesign3_ABE_NGG_ | mAsmCsmCsCAGGGGAACAACAGAAGGUUUUAGAGCUAGA | 806 |
| 20nt_3- | AAUAGCAAGUUAAAAUAAGGCUAGUCCGUUAUCAACUUGA | |
| 12_018_+_31947938_spCas9 | AAAAGUGGCACCGAGUCGGUGCUsmUsmUsmU | |
| GuideDesign3_ABE_NGG_ | mCsmAsmCsCCACAGGUGGCAAGUUAGUUUUAGAGCUAGA | 807 |
| 20nt_3- | AAUAGCAAGUUAAAAUAAGGCUAGUCCGUUAUCAACUUGA | |
| 12_018_+_31948364_spCas9 | AAAAGUGGCACCGAGUCGGUGCUsmUsmUsmU | |
| GuideDesign3_ABE_NGG_ | mAsmCsmAsGACCACAAGUUGAAGUCGUUUUAGAGCUAGA | 808 |
| 20nt_3- | AAUAGCAAGUUAAAAUAAGGCUAGUCCGUUAUCAACUUGA | |
| 12_018_+_31948825_spCas9 | AAAAGUGGCACCGAGUCGGUGCUsmUsmUsmU | |
| GuideDesign3_ABE_NGG_ | mUsmUsmCsUGCAGGAUUGCACAACAGUUUUAGAGCUAGA | 809 |
| 20nt_3- | AAUAGCAAGUUAAAAUAAGGCUAGUCCGUUAUCAACUUGA | |
| 12_018_+_31949234_spCas9 | AAAAGUGGCACCGAGUCGGUGCUsmUsmUsmU | |
| GuideDesign3_ABE_NGG_ | mUsmCsmUsGCAGGAUUGCACAACAUGUUUUAGAGCUAGA | 810 |
| 20nt_3- | AAUAGCAAGUUAAAAUAAGGCUAGUCCGUUAUCAACUUGA | |
| 12_018_+_31949235_spCas9 | AAAAGUGGCACCGAGUCGGUGCUsmUsmUsmU | |
| GuideDesign3_ABE_NGG_ | mGsmCsmAsGGAUUGCACAACAUGGGGUUUUAGAGCUAGA | 811 |
| 20nt_3- | AAUAGCAAGUUAAAAUAAGGCUAGUCCGUUAUCAACUUGA | |
| 12_018_+_31949238_spCas9 | AAAAGUGGCACCGAGUCGGUGCUsmUsmUsmU | |
| GuideDesign3_ABE_NGG_ | mCsmCsmCsUCAGAUGUCUAUGUGUUGUUUUAGAGCUAGA | 812 |
| 20nt_3- | AAUAGCAAGUUAAAAUAAGGCUAGUCCGUUAUCAACUUGA | |
| 12_018_+_31949412_spCas9 | AAAAGUGGCACCGAGUCGGUGCUsmUsmUsmU | |
| GuideDesign3_ABE_NGG_ | mCsmCsmUsCAGAUGUCUAUGUGUUUGUUUUAGAGCUAGA | 813 |
| 20nt_3- | AAUAGCAAGUUAAAAUAAGGCUAGUCCGUUAUCAACUUGA | |
| 12_018_+_31949413_spCas9 | AAAAGUGGCACCGAGUCGGUGCUsmUsmUsmU | |
| GuideDesign3_ABE_NGG_ | mCsmUsmCsAGAUGUCUAUGUGUUUGGUUUUAGAGCUAGA | 814 |
| 20nt_3- | AAUAGCAAGUUAAAAUAAGGCUAGUCCGUUAUCAACUUGA | |
| 12_018_+_31949414_spCas9 | AAAAGUGGCACCGAGUCGGUGCUsmUsmUsmU | |
| GuideDesign3_ABE_NGG_ | mGsmUsmGsUCCCCAGCGCCCUUCAAGUUUUAGAGCUAGA | 815 |
| 20nt_3- | AAUAGCAAGUUAAAAUAAGGCUAGUCCGUUAUCAACUUGA | |
| 12_018_+_31950275_spCas9 | AAAAGUGGCACCGAGUCGGUGCUsmUsmUsmU | |
| GuideDesign3_ABE_NGG_ | mUsmGsmUsCCCCAGCGCCCUUCAAAGUUUUAGAGCUAGA | 816 |
| 20nt_3- | AAUAGCAAGUUAAAAUAAGGCUAGUCCGUUAUCAACUUGA | |
| 12_018_+_31950276_spCas9 | AAAAGUGGCACCGAGUCGGUGCUsmUsmUsmU | |
| GuideDesign3_ABE_NGG_ | mCsmUsmAsCUUCAGGAGGGGAGAAGGUUUUAGAGCUAGA | 817 |
| 20nt_3- | AAUAGCAAGUUAAAAUAAGGCUAGUCCGUUAUCAACUUGA | |
| 12_018_+_31950609_spCas9 | AAAAGUGGCACCGAGUCGGUGCUsmUsmUsmU | |
| GuideDesign3_ABE_NGG_ | mUsmAsmCsUUCAGGAGGGGAGAAGCGUUUUAGAGCUAGA | 818 |
| 20nt_3- | AAUAGCAAGUUAAAAUAAGGCUAGUCCGUUAUCAACUUGA | |
| 12_018_+_31950610_spCas9 | AAAAGUGGCACCGAGUCGGUGCUsmUsmUsmU | |
| GuideDesign3_ABE_NGG_ | mCsmCsmUsCUCCUUGCAGGUGAUUCGUUUUAGAGCUAGA | 819 |
| 20nt_3- | AAUAGCAAGUUAAAAUAAGGCUAGUCCGUUAUCAACUUGA | |
| 12_018_+_31951541_spCas9 | AAAAGUGGCACCGAGUCGGUGCUsmUsmUsmU | |
| GuideDesign3_ABE_NGG_ | mCsmUsmCsCUUGCAGGUGAUUCUGGGUUUUAGAGCUAGA | 820 |
| 20nt_3- | AAUAGCAAGUUAAAAUAAGGCUAGUCCGUUAUCAACUUGA | |
| 12_018_+_31951544_spCas9 | AAAAGUGGCACCGAGUCGGUGCUsmUsmUsmU | |
| GuideDesign3_ABE_NGG_ | mUsmGsmCsAGGUUGGUGUAAUCAGCGUUUUAGAGCUAGA | 821 |
| 20nt_3- | AAUAGCAAGUUAAAAUAAGGCUAGUCCGUUAUCAACUUGA | |
| 12_018_+_31951869_spCas9 | AAAAGUGGCACCGAGUCGGUGCUsmUsmUsmU | |
| GuideDesign3_ABE_NGG_ | mGsmCsmAsGGUUGGUGUAAUCAGCUGUUUUAGAGCUAGA | 822 |
| 20nt_3- | AAUAGCAAGUUAAAAUAAGGCUAGUCCGUUAUCAACUUGA | |
| 12_018_+_31951870_spCas9 | AAAAGUGGCACCGAGUCGGUGCUsmUsmUsmU | |
| GuideDesign3_ABE_NGG_ | mCsmUsmCsUCACCUCUGCAAGUAUUGUUUUAGAGCUAGA | 823 |
| 20nt_3-9_002_- | AAUAGCAAGUUAAAAUAAGGCUAGUCCGUUAUCAACUUGA | |
| 31951457_spCas9 | AAAAGUGGCACCGAGUCGGUGCUsmUsmUsmU | |
| GuideDesign3_ABE_NGG_ | mUsmCsmUsCUCACCUCUGCAAGUAUGUUUUAGAGCUAGA | 824 |
| 20nt_3-9_002- | AAUAGCAAGUUAAAAUAAGGCUAGUCCGUUAUCAACUUGA | |
| 31951458_spCas9 | AAAAGUGGCACCGAGUCGGUGCUsmUsmUsmU | |
| GuideDesign3_ABE_NGG_ | mAsmCsmCsCAGGGGAACAACAGAAGGUUUUAGAGCUAGA | 825 |
| 20nt_3- | AAUAGCAAGUUAAAAUAAGGCUAGUCCGUUAUCAACUUGA | |
| 9_002_+_31947938_spCas9 | AAAAGUGGCACCGAGUCGGUGCUsmUsmUsmU | |
| GuideDesign3_ABE_NGG_ | mUsmUsmCsUGCAGGAUUGCACAACAGUUUUAGAGCUAGA | 826 |
| 20nt_3- | AAUAGCAAGUUAAAAUAAGGCUAGUCCGUUAUCAACUUGA | |
| 9_002_+_31949234_spCas9 | AAAAGUGGCACCGAGUCGGUGCUsmUsmUsmU | |
| GuideDesign3_ABE_NNNRRT_ | mUsmCsmCsCUUCUGACCAUCAGUCAUGUUUUAGUACUCU | 827 |
| 21nt_5-14_014- | GUAAUGAAAAUUACAGAAUCUACUAAAACAAGGCAAAAUG | |
| 31948945_saCas9 | CCGUGUUUAUCUCGUCAACUUGUUGGCGAGAUsmUsmUsm | |
| | U | |
| GuideDesign3_ABE_NNNRRT_ | mAsmCsmUsCACCCAGAUAAUCCUCCCGUUUUAGUACUCU | 828 |
| 21nt_5-14_014- | GUAAUGAAAAUUACAGAAUCUACUAAAACAAGGCAAAAUG | |
| 31949323_saCas9 | CCGUGUUUAUCUCGUCAACUUGUUGGCGAGAUsmUsmUsm | |
| | U | |
| GuideDesign3_ABE_NNNRRT_ | mCsmCsmUsUACCUACGCUGACCUUGAGUUUUAGUACUCU | 829 |
| 21nt_5-14_014- | GUAAUGAAAAUUACAGAAUCUACUAAAACAAGGCAAAAUG | |
| 31950382_saCas9 | CCGUGUUUAUCUCGUCAACUUGUUGGCGAGAUsmUsmUsm | |
| | U | |
| GuideDesign3_ABE_NNNRRT_ | mGsmUsmUsUCUCACCUUAUCCCCAUUGUUUUAGUACUCU | 830 |
| 21nt_5-14_014- | GUAAUGAAAAUUACAGAAUCUACUAAAACAAGGCAAAAUG | |
| 31951226_saCas9 | CCGUGUUUAUCUCGUCAACUUGUUGGCGAGAUsmUsmUsm | |
| | U | |
| GuideDesign3_ABE_NNNRRT_ | mUsmUsmCsUCUCACCUCUGCAAGUAUGUUUUAGUACUCU | 831 |
| 21nt_5-14_014- | GUAAUGAAAAUUACAGAAUCUACUAAAACAAGGCAAAAUG | |
| 31951455_saCas9 | CCGUGUUUAUCUCGUCAACUUGUUGGCGAGAUsmUsmUsm | |
| | U | |
| GuideDesign3_ABE_NNNRRT_ | mCsmCsmUsCACCCACAGGUGGCAAGUGUUUUAGUACUCU | 832 |
| 21nt_5- | GUAAUGAAAAUUACAGAAUCUACUAAAACAAGGCAAAAUG | |
| 14_014_+_31948361_saCas9 | CCGUGUUUAUCUCGUCAACUUGUUGGCGAGAUsmUsmUsm | |
| | U | |
| GuideDesign3_ABE_NNNRRT_ | mUsmCsmCsCUCAGAUGUCUAUGUGUUGUUUUAGUACUCU | 833 |
| 21nt_5- | GUAAUGAAAAUUACAGAAUCUACUAAAACAAGGCAAAAUG | |
| 14_014_+_31949411_saCas9 | CCGUGUUUAUCUCGUCAACUUGUUGGCGAGAUsmUsmUsm | |
| | U | |
| GuideDesign3_ABE_NNNRRT_ | mCsmUsmCsCUUCUCUGCCAGAUGAAAGUUUUAGUACUCU | 834 |
| 21nt_5- | GUAAUGAAAAUUACAGAAUCUACUAAAACAAGGCAAAAUG | |
| 14_014_+_31950034_saCas9 | CCGUGUUUAUCUCGUCAACUUGUUGGCGAGAUsmUsmUsm | |
| | U | |
| GuideDesign3_ABE_NNNRRT_ | mCsmUsmGsCCAGAUGAAAGCCAGUCUGUUUUAGUACUCU | 835 |
| 21nt_5- | GUAAUGAAAAUUACAGAAUCUACUAAAACAAGGCAAAAUG | |
| 14_014_+_31950042_saCas9 | CCGUGUUUAUCUCGUCAACUUGUUGGCGAGAUsmUsmUsm | |
| | U | |
| GuideDesign3_ABE_VTTN 2 | mGsmUsmUsCUGUCUUUUGGUCAGGACAACCGUCUAGCUA | 836 |
| 20nt_5-9_017_- | UAAGUGCUGCAGGGUGUGAGAAACUCCUAUUGCUGGACGA | |
| 31946593_cas12b | UGUCUCUUACGAGGCAUUAGCACCCCUCAAACCUCUGCAC | |
| | UCsmUsmGsmC | |
| GuideDesign3_ABE_VTTN_ | mGsmUsmUsCUGUCUUUUGGUCAGGACAACCGUCUAGCUA | 837 |
| 20nt_5-9_017- | UAAGUGCUGCAGGGUGUGAGAAACUCCUAUUGCUGGACGA | |
| 31950134_cas12b | UGUCUCUUACGAGGCAUUAGCACUGUGCUUACAAUGACUG | |
| | AGsmAsmUsmC | |
| GuideDesign3_ABE_VTTN_ | mGsmUsmUsCUGUCUUUUGGUCAGGACAACCGUCUAGCUA | 838 |
| 20nt_5-9_017_- | UAAGUGCUGCAGGGUGUGAGAAACUCCUAUUGCUGGACGA | |
| 31950390_cas12b | UGUCUCUUACGAGGCAUUAGCACCAUCCUUACCUACGCUG | |
| | ACsmCsmUsmU | |
| GuideDesign3_ABE_VTTN 2 | mGsmUsmUsCUGUCUUUUGGUCAGGACAACCGUCUAGCUA | 839 |
| 20nt_5-9_017- | UAAGUGCUGCAGGGUGUGAGAAACUCCUAUUGCUGGACGA | |
| 31951457_cas12b | UGUCUCUUACGAGGCAUUAGCACUCUCACCUCUGCAAGUA | |
| | UUsmGsmGsmG | |
| GuideDesign3_ABE_VTTN_ | mGsmUsmUsCUGUCUUUUGGUCAGGACAACCGUCUAGCUA | 840 |
| 20nt_5- | UAAGUGCUGCAGGGUGUGAGAAACUCCUAUUGCUGGACGA | |
| 9_017_+_31946360_cas12b | UGUCUCUUACGAGGCAUUAGCACCUCUUCAGGUGUGACCA | |
| | CCsmAsmCsmU | |
| GuideDesign3_ABE_VTTN_ | mGsmUsmUsCUGUCUUUUGGUCAGGACAACCGUCUAGCUA | 841 |
| 20nt_5- | UAAGUGCUGCAGGGUGUGAGAAACUCCUAUUGCUGGACGA | |
| 9_017_+_31946996_cas12b | UGUCUCUUACGAGGCAUUAGCACUCAAAGCAAUCCACUGU | |
| | CCsmAsmAsmG | |
| GuideDesign3_ABE_VTTN 2 | mGsmUsmUsCUGUCUUUUGGUCAGGACAACCGUCUAGCUA | 842 |
| 20nt_5- | UAAGUGCUGCAGGGUGUGAGAAACUCCUAUUGCUGGACGA | |
| 9_017_+_31950037_cas12b | UGUCUCUUACGAGGCAUUAGCACUCUGCCAGAUGAAAGCC | |
| | AGsmUsmCsmU | |
| GuideDesign3_ABE_VTTN 2 | mGsmUsmUsCUGUCUUUUGGUCAGGACAACCGUCUAGCUA | 843 |
| 20nt_5- | UAAGUGCUGCAGGGUGUGAGAAACUCCUAUUGCUGGACGA | |
| 9_017_+_31950272_cas12b | UGUCUCUUACGAGGCAUUAGCACUGUCCCCAGCGCCCUUC | |
| | AAsmAsmGsmG | |
| GuideDesign3_ABE_VTTN_ | mGsmUsmUsCUGUCUUUUGGUCAGGACAACCGUCUAGCUA | 844 |
| 20nt_5- | UAAGUGCUGCAGGGUGUGAGAAACUCCUAUUGCUGGACGA | |
| 9_017_+_31950853_cas12b | UGUCUCUUACGAGGCAUUAGCACUCCUUUAUAGGCCCAUU | |
| | UGsmUsmCsmU | |
| GuideDesign3_ABE_VTTN_ | mGsmUsmUsCUGUCUUUUGGUCAGGACAACCGUCUAGCUA | 845 |
| 20nt_5- | UAAGUGCUGCAGGGUGUGAGAAACUCCUAUUGCUGGACGA | |
| 9_017_+_31951133_cas12b | UGUCUCUUACGAGGCAUUAGCACGUCCAGAGGAAGAGCUG | |
| | CUsmCsmCsmC | |
| GuideDesign3_CBE_NNGRR | mUsmCsmCsCUUCUGACCAUCAGUCAUGUUUUAGUACUCU | 846 |
| 21nt_3-12_012- | GUAAUGAAAAUUACAGAAUCUACUAAAACAAGGCAAAAUG | |
| 31948945_saCas9 | CCGUGUUUAUCUCGUCAACUUGUUGGCGAGAUsmUsmUsm | |
| | U | |
| GuideDesign3_CBE_NNGRR | mAsmCsmUsCACCCAGAUAAUCCUCCCGUUUUAGUACUCU | 847 |
| 21nt_3-12_012- | GUAAUGAAAAUUACAGAAUCUACUAAAACAAGGCAAAAUG | |
| 31949323_saCas9 | CCGUGUUUAUCUCGUCAACUUGUUGGCGAGAUsmUsmUsm | |
| | U | |
| GuideDesign3_CBE_NNGRR | mCsmCsmUsUACCUACGCUGACCUUGAGUUUUAGUACUCU | 848 |
| 21nt_3-12_012_- | GUAAUGAAAAUUACAGAAUCUACUAAAACAAGGCAAAAUG | |
| 31950382_saCas9 | CCGUGUUUAUCUCGUCAACUUGUUGGCGAGAUsmUsmUsm | |
| | U | |
| GuideDesign3_CBE_NNGRR | mUsmUsmCsUCUCACCUCUGCAAGUAUGUUUUAGUACUCU | 849 |
| 21nt_3-12_012- | GUAAUGAAAAUUACAGAAUCUACUAAAACAAGGCAAAAUG | |
| 31951455_saCas9 | CCGUGUUUAUCUCGUCAACUUGUUGGCGAGAUsmUsmUsm | |
| | U | |
| GuideDesign3_CBE_NNNRRT_ | mAsmUsmGsCUUCUCACCUCCGUUGUCGUUUUAGUACUCU | 850 |
| 21nt_3-12_015- | GUAAUGAAAAUUACAGAAUCUACUAAAACAAGGCAAAAUG | |
| 31947176_saCas9 | CCGUGUUUAUCUCGUCAACUUGUUGGCGAGAUsmUsmUsm | |
| | U | |
| GuideDesign3_CBE_NNNRRT_ | mUsmGsmUsUGUUCCCCUGGGUGCCAGGUUUUAGUACUCU | 851 |
| 21nt_3-12_015- | GUAAUGAAAAUUACAGAAUCUACUAAAACAAGGCAAAAUG | |
| 31947927_saCas9 | CCGUGUUUAUCUCGUCAACUUGUUGGCGAGAUsmUsmUsm | |
| | U | |
| GuideDesign3_CBE_NNNRRT_ | mCsmUsmUsCUGACCAUCAGUCAUGAGGUUUUAGUACUCU | 852 |
| 21nt_3-12_015- | GUAAUGAAAAUUACAGAAUCUACUAAAACAAGGCAAAAUG | |
| 31948942_saCas9 | CCGUGUUUAUCUCGUCAACUUGUUGGCGAGAUsmUsmUsm | |
| | U | |
| GuideDesign3_CBE_NNNRRT_ | mUsmCsmCsCUUCUGACCAUCAGUCAUGUUUUAGUACUCU | 853 |
| 21nt_3-12_015- | GUAAUGAAAAUUACAGAAUCUACUAAAACAAGGCAAAAUG | |
| 31948945_saCas9 | CCGUGUUUAUCUCGUCAACUUGUUGGCGAGAUsmUsmUsm | |
| | U | |
| GuideDesign3_CBE_NNNRRT_ | mAsmCsmUsCACCCAGAUAAUCCUCCCGUUUUAGUACUCU | 854 |
| 21nt_3-12_015- | GUAAUGAAAAUUACAGAAUCUACUAAAACAAGGCAAAAUG | |
| 31949323_saCas9 | CCGUGUUUAUCUCGUCAACUUGUUGGCGAGAUsmUsmUsm | |
| | U | |
| GuideDesign3_CBE_NNNRRT_ | mGsmAsmCsAUCUGAGGGAUAAAAAGGGUUUUAGUACUCU | 855 |
| 21nt_3-12_015- | GUAAUGAAAAUUACAGAAUCUACUAAAACAAGGCAAAAUG | |
| 31949397_saCas9 | CCGUGUUUAUCUCGUCAACUUGUUGGCGAGAUsmUsmUsm | |
| | U | |
| GuideDesign3_CBE_NNNRRT_ | mGsmGsmCsUUUCAUCUGGCAGAGAAGGUUUUAGUACUCU | 856 |
| 21nt_3-12_015- | GUAAUGAAAAUUACAGAAUCUACUAAAACAAGGCAAAAUG | |
| 31950031_saCas9 | CCGUGUUUAUCUCGUCAACUUGUUGGCGAGAUsmUsmUsm | |
| | U | |
| GuideDesign3_CBE_NNNRRT_ | mCsmCsmUsUACCUACGCUGACCUUGAGUUUUAGUACUCU | 857 |
| 21nt_3-12_015- | GUAAUGAAAAUUACAGAAUCUACUAAAACAAGGCAAAAUG | |
| 31950382_saCas9 | CCGUGUUUAUCUCGUCAACUUGUUGGCGAGAUsmUsmUsm | |
| | U | |
| GuideDesign3_CBE_NNNRRT_ | mUsmGsmCsAUCCUUACCUACGCUGACGUUUUAGUACUCU | 858 |
| 21nt_3-12_015- | GUAAUGAAAAUUACAGAAUCUACUAAAACAAGGCAAAAUG | |
| 31950387_saCas9 | CCGUGUUUAUCUCGUCAACUUGUUGGCGAGAUsmUsmUsm | |
| | U | |
| GuideDesign3_CBE_NNNRRT_ | mUsmGsmUsCUUACUUUGUUGCUGGCAGUUUUAGUACUCU | 859 |
| 21nt_3-12_015_- | GUAAUGAAAAUUACAGAAUCUACUAAAACAAGGCAAAAUG | |
| 31950925_saCas9 | CCGUGUUUAUCUCGUCAACUUGUUGGCGAGAUsmUsmUsm | |
| | U | |
| GuideDesign3_CBE_NNNRRT_ | mGsmUsmAsUGUCUUACUUUGUUGCUGGUUUUAGUACUCU | 860 |
| 21nt_3-12_015- | GUAAUGAAAAUUACAGAAUCUACUAAAACAAGGCAAAAUG | |
| 31950928_saCas9 | CCGUGUUUAUCUCGUCAACUUGUUGGCGAGAUsmUsmUsm | |
| | U | |
| GuideDesign3_CBE_NNNRRT_ | mGsmUsmUsUCUCACCUUAUCCCCAUUGUUUUAGUACUCU | 861 |
| 21nt_3-12_015- | GUAAUGAAAAUUACAGAAUCUACUAAAACAAGGCAAAAUG | |
| 31951226_saCas9 | CCGUGUUUAUCUCGUCAACUUGUUGGCGAGAUsmUsmUsm | |
| | U | |
| GuideDesign3_CBE_NNNRRT_ | mUsmUsmCsUCUCACCUCUGCAAGUAUGUUUUAGUACUCU | 862 |
| 21nt_3-12_015- | GUAAUGAAAAUUACAGAAUCUACUAAAACAAGGCAAAAUG | |
| 31951455_saCas9 | CCGUGUUUAUCUCGUCAACUUGUUGGCGAGAUsmUsmUsm | |
| | U | |
| GuideDesign3_CBE_NNNRRT_ | mUsmAsmCsACCAACCUGCAGAGGCAGGUUUUAGUACUCU | 863 |
| 21nt_3-12_015- | GUAAUGAAAAUUACAGAAUCUACUAAAACAAGGCAAAAUG | |
| 31951856_saCas9 | CCGUGUUUAUCUCGUCAACUUGUUGGCGAGAUsmUsmUsm | |
| | U | |
| GuideDesign10_CBE_NGA_ | mCsmAsmCsCUGAAGAGAAAGGCUGAGUUUUAGAGCUAGA | 864 |
| 20nt_4-9_006- | AAUAGCAAGUUAAAAUAAGGCUAGUCCGUUAUCAACUUGA | |
| 31946352_spCas9 | AAAAGUGGCACCGAGUCGGUGCUsmUsmUsmU | |
| GuideDesign10_CBE_NGA_ | mUsmGsmGsAUUGCUUUGAGAAGGGGGUUUUAGAGCUAGA | 865 |
| 20nt_4-9_006- | AAUAGCAAGUUAAAAUAAGGCUAGUCCGUUAUCAACUUGA | |
| 31946990_spCas9 | AAAAGUGGCACCGAGUCGGUGCUsmUsmUsmU | |
| GuideDesign10_CBE_NGA_ | mUsmAsmCsCCCGCUGCAGAGGUAUGGUUUUAGAGCUAGA | 866 |
| 20nt_4-9_006- | AAUAGCAAGUUAAAAUAAGGCUAGUCCGUUAUCAACUUGA | |
| 31947331_spCas9 | AAAAGUGGCACCGAGUCGGUGCUsmUsmUsmU | |
| GuideDesign10_CBE_NGA_ | mGsmAsmGsUCUGGGAGAGUCAGAAAGUUUUAGAGCUAGA | 867 |
| 20nt_4-9_006- | AAUAGCAAGUUAAAAUAAGGCUAGUCCGUUAUCAACUUGA | |
| 31947722_spCas9 | AAAAGUGGCACCGAGUCGGUGCUsmUsmUsmU | |
| GuideDesign10_CBE_NGA_ | mUsmGsmGsGCCUAUAAAGGACAAGGGUUUUAGAGCUAGA | 868 |
| 20nt_4-9_006- | AAUAGCAAGUUAAAAUAAGGCUAGUCCGUUAUCAACUUGA | |
| 31950849_spCas9 | AAAAGUGGCACCGAGUCGGUGCUsmUsmUsmU | |
| GuideDesign10_CBE_NGA_ | mAsmAsmUsGGGCCUAUAAAGGACAAGUUUUAGAGCUAGA | 869 |
| 20nt_4-9_006- | AAUAGCAAGUUAAAAUAAGGCUAGUCCGUUAUCAACUUGA | |
| 31950851_spCas9 | AAAAGUGGCACCGAGUCGGUGCUsmUsmUsmU | |
| GuideDesign10_CBE_NGA_ | mGsmCsmCsUUUCUGUAGGGGAGGUGGUUUUAGAGCUAGA | 870 |
| 20nt_4-9_006- | AAUAGCAAGUUAAAAUAAGGCUAGUCCGUUAUCAACUUGA | |
| 31951323_spCas9 | AAAAGUGGCACCGAGUCGGUGCUsmUsmUsmU | |
| GuideDesign10_CBE_NGG_ | mGsmUsmGsGAUUGCUUUGAGAAGGGGUUUUAGAGCUAGA | 871 |
| 20nt_4-9_003- | AAUAGCAAGUUAAAAUAAGGCUAGUCCGUUAUCAACUUGA | |
| 31946991_spCas9 | AAAAGUGGCACCGAGUCGGUGCUsmUsmUsmU | |
| GuideDesign10_CBE_NGG_ | mUsmGsmCsAAUCCUGCAGAAGAGACGUUUUAGAGCUAGA | 872 |
| 20nt_4-9_003- | AAUAGCAAGUUAAAAUAAGGCUAGUCCGUUAUCAACUUGA | |
| 31949226_spCas9 | AAAAGUGGCACCGAGUCGGUGCUsmUsmUsmU | |
| GuideDesign10_CBE_NGG_ | mCsmAsmUsCUGAGGGAUAAAAAGGAGUUUUAGAGCUAGA | 873 |
| 20nt_4-9_003- | AAUAGCAAGUUAAAAUAAGGCUAGUCCGUUAUCAACUUGA | |
| 31949399_spCas9 | AAAAGUGGCACCGAGUCGGUGCUsmUsmUsmU | |
| GuideDesign10_CBE_NGG_ | mUsmAsmGsACAUCUGAGGGAUAAAAGUUUUAGAGCUAGA | 874 |
| 20nt_4-9_003- | AAUAGCAAGUUAAAAUAAGGCUAGUCCGUUAUCAACUUGA | |
| 31949403_spCas9 | AAAAGUGGCACCGAGUCGGUGCUsmUsmUsmU | |
| GuideDesign10_CBE_NGG_ | mCsmCsmCsUCCUGAAGUAGGAGAGUGUUUUAGAGCUAGA | 875 |
| 20nt_4-9_003- | AAUAGCAAGUUAAAAUAAGGCUAGUCCGUUAUCAACUUGA | |
| 31950600_spCas9 | AAAAGUGGCACCGAGUCGGUGCUsmUsmUsmU | |
| GuideDesign10_CBE_NGG_ | mAsmAsmAsUGGGCCUAUAAAGGACAGUUUUAGAGCUAGA | 876 |
| 20nt_4-9_003- | AAUAGCAAGUUAAAAUAAGGCUAGUCCGUUAUCAACUUGA | |
| 31950852_spCas9 | AAAAGUGGCACCGAGUCGGUGCUsmUsmUsmU | |
| GuideDesign10_CBE_NGG_ | mUsmGsmCsCUUUCUGUAGGGGAGGUGUUUUAGAGCUAGA | 877 |
| 20nt_4-9_003- | AAUAGCAAGUUAAAAUAAGGCUAGUCCGUUAUCAACUUGA | |
| 31951324_spCas9 | AAAAGUGGCACCGAGUCGGUGCUsmUsmUsmU | |
| GuideDesign10_CBE_NGG_ | mAsmCsmCsAACCUGCAGAGGCAGUGGUUUUAGAGCUAGA | 878 |
| 20nt_4-9_003- | AAUAGCAAGUUAAAAUAAGGCUAGUCCGUUAUCAACUUGA | |
| 31951857_spCas9 | AAAAGUGGCACCGAGUCGGUGCUsmUsmUsmU | |
| GuideDesign3_ABE_NGA_ | mCsmUsmUsCUCACCUCCGUUGUCACGUUUUAGAGCUAGA | 879 |
| 20nt_3-9_005- | AAUAGCAAGUUAAAAUAAGGCUAGUCCGUUAUCAACUUGA | |
| 31947177_spCas9 | AAAAGUGGCACCGAGUCGGUGCUsmUsmUsmU | |
| GuideDesign3_ABE_NGA_ | mCsmUsmUsCUCACCUCCGUUGUCACGUUUUAGAGCUAGA | 880 |
| 20nt_3-9_005- | AAUAGCAAGUUAAAAUAAGGCUAGUCCGUUAUCAACUUGA | |
| 31947177_spCas9 | AAAAGUGGCACCGAGUCGGUGCUsmUsmUsmU | |
| GuideDesign3_ABE_NGA_ | mCsmUsmGsACCAUCAGUCAUGAGGAGUUUUAGAGCUAGA | 881 |
| 20nt_3-9_005- | AAUAGCAAGUUAAAAUAAGGCUAGUCCGUUAUCAACUUGA | |
| 31948943_spCas9 | AAAAGUGGCACCGAGUCGGUGCUsmUsmUsmU | |
| GuideDesign3_ABE_NGA_ | mCsmUsmGsACCAUCAGUCAUGAGGAGUUUUAGAGCUAGA | 882 |
| 20nt_3-9_005- | AAUAGCAAGUUAAAAUAAGGCUAGUCCGUUAUCAACUUGA | |
| 31948943_spCas9 | AAAAGUGGCACCGAGUCGGUGCUsmUsmUsmU | |
| GuideDesign3_ABE_NGA_ | mCsmUsmCsCCUACCGAUCAUUUGGUGUUUUAGAGCUAGA | 883 |
| 20nt_3-9_005- | AAUAGCAAGUUAAAAUAAGGCUAGUCCGUUAUCAACUUGA | |
| 31949542_spCas9 | AAAAGUGGCACCGAGUCGGUGCUsmUsmUsmU | |
| GuideDesign3_ABE_NGA_ | mCsmUsmCsCCUACCGAUCAUUUGGUGUUUUAGAGCUAGA | 884 |
| 20nt_3-9_005- | AAUAGCAAGUUAAAAUAAGGCUAGUCCGUUAUCAACUUGA | |
| 31949542_spCas9 | AAAAGUGGCACCGAGUCGGUGCUsmUsmUsmU | |
| GuideDesign3_ABE_NGA_ | mUsmUsmAsCCUACGCUGACCUUGAUGUUUUAGAGCUAGA | 885 |
| 20nt_3-9_005- | AAUAGCAAGUUAAAAUAAGGCUAGUCCGUUAUCAACUUGA | |
| 31950384_spCas9 | AAAAGUGGCACCGAGUCGGUGCUsmUsmUsmU | |
| GuideDesign3_ABE_NGA_ | mUsmUsmAsCCUACGCUGACCUUGAUGUUUUAGAGCUAGA | 886 |
| 20nt_3-9_005- | AAUAGCAAGUUAAAAUAAGGCUAGUCCGUUAUCAACUUGA | |
| 31950384_spCas9 | AAAAGUGGCACCGAGUCGGUGCUsmUsmUsmU | |
| GuideDesign3_ABE_NGA_ | mAsmUsmCsCUUACCUACGCUGACCUGUUUUAGAGCUAGA | 887 |
| 20nt_3-9_005- | AAUAGCAAGUUAAAAUAAGGCUAGUCCGUUAUCAACUUGA | |
| 31950388_spCas9 | AAAAGUGGCACCGAGUCGGUGCUsmUsmUsmU | |
| GuideDesign3_ABE_NGA_ | mAsmUsmCsCUUACCUACGCUGACCUGUUUUAGAGCUAGA | 888 |
| 20nt_3-9_005- | AAUAGCAAGUUAAAAUAAGGCUAGUCCGUUAUCAACUUGA | |
| 31950388_spCas9 | AAAAGUGGCACCGAGUCGGUGCUsmUsmUsmU | |
| GuideDesign3_ABE_NGA_ | mUsmCsmUsCACCUUAUCCCCAUUCUGUUUUAGAGCUAGA | 889 |
| 20nt_3-9_005- | AAUAGCAAGUUAAAAUAAGGCUAGUCCGUUAUCAACUUGA | |
| 31951227_spCas9 | AAAAGUGGCACCGAGUCGGUGCUsmUsmUsmU | |
| GuideDesign3_ABE_NGA_ | mUsmCsmUsCACCUUAUCCCCAUUCUGUUUUAGAGCUAGA | 890 |
| 20nt_3-9_005_- | AAUAGCAAGUUAAAAUAAGGCUAGUCCGUUAUCAACUUGA | |
| 31951227_spCas9 | AAAAGUGGCACCGAGUCGGUGCUsmUsmUsmU | |
| GuideDesign3_ABE_NGA_ | mGsmAsmGsGACUCACUUGAAUGAAAGUUUUAGAGCUAGA | 891 |
| 20nt_3-9_005- | AAUAGCAAGUUAAAAUAAGGCUAGUCCGUUAUCAACUUGA | |
| 31951591_spCas9 | AAAAGUGGCACCGAGUCGGUGCUsmUsmUsmU | |
| GuideDesign3_ABE_NGA_ | mGsmAsmGsGACUCACUUGAAUGAAAGUUUUAGAGCUAGA | 892 |
| 20nt_3-9_005- | AAUAGCAAGUUAAAAUAAGGCUAGUCCGUUAUCAACUUGA | |
| 31951591_spCas9 | AAAAGUGGCACCGAGUCGGUGCUsmUsmUsmU | |
| GuideDesign3_ABE_NGA_ | mUsmCsmAsAAGCAAUCCACUGUCCAGUUUUAGAGCUAGA | 893 |
| 20nt_3- | AAUAGCAAGUUAAAAUAAGGCUAGUCCGUUAUCAACUUGA | |
| 9_005_+_31947000_spCas9 | AAAAGUGGCACCGAGUCGGUGCUsmUsmUsmU | |
| GuideDesign3_ABE_NGA_ | mUsmCsmAsAAGCAAUCCACUGUCCAGUUUUAGAGCUAGA | 894 |
| 20nt_3- | AAUAGCAAGUUAAAAUAAGGCUAGUCCGUUAUCAACUUGA | |
| 9_005_+_31947000_spCas9 | AAAAGUGGCACCGAGUCGGUGCUsmUsmUsmU | |
| GuideDesign3_ABE_NGA_ | mCsmUsmCsCCAGACUCCUUCAUGUAGUUUUAGAGCUAGA | 895 |
| 20nt_3- | AAUAGCAAGUUAAAAUAAGGCUAGUCCGUUAUCAACUUGA | |
| 9_005_+_31947734_spCas9 | AAAAGUGGCACCGAGUCGGUGCUsmUsmUsmU | |
| GuideDesign3_ABE_NGA_ | mCsmUsmCsCCAGACUCCUUCAUGUAGUUUUAGAGCUAGA | 896 |
| 20nt_3- | AAUAGCAAGUUAAAAUAAGGCUAGUCCGUUAUCAACUUGA | |
| 9_005_+_31947734_spCas9 | AAAAGUGGCACCGAGUCGGUGCUsmUsmUsmU | |
| GuideDesign3_ABE_NGA_ | mCsmCsmCsAGGGGAACAACAGAAGCGUUUUAGAGCUAGA | 897 |
| 20nt_3- | AAUAGCAAGUUAAAAUAAGGCUAGUCCGUUAUCAACUUGA | |
| 9_005_+_31947939_spCas9 | AAAAGUGGCACCGAGUCGGUGCUsmUsmUsmU | |
| GuideDesign3_ABE_NGA_ | mCsmCsmCsAGGGGAACAACAGAAGCGUUUUAGAGCUAGA | 898 |
| 20nt_3- | AAUAGCAAGUUAAAAUAAGGCUAGUCCGUUAUCAACUUGA | |
| 9_005_+_31947939_spCas9 | AAAAGUGGCACCGAGUCGGUGCUsmUsmUsmU | |
| GuideDesign3_ABE_NGA_ | mAsmCsmAsGGUGGCAAGUUAUGGUGGUUUUAGAGCUAGA | 899 |
| 20nt_3- | AAUAGCAAGUUAAAAUAAGGCUAGUCCGUUAUCAACUUGA | |
| 9_005_+_31948369_spCas9 | AAAAGUGGCACCGAGUCGGUGCUsmUsmUsmU | |
| GuideDesign3_ABE_NGA_ | mAsmCsmAsGGUGGCAAGUUAUGGUGGUUUUAGAGCUAGA | 900 |
| 20nt_3- | AAUAGCAAGUUAAAAUAAGGCUAGUCCGUUAUCAACUUGA | |
| 9_005_+_31948369_spCas9 | AAAAGUGGCACCGAGUCGGUGCUsmUsmUsmU | |
| GuideDesign3_ABE_NGA_ | mGsmCsmCsAGAUGAAAGCCAGUCUCGUUUUAGAGCUAGA | 901 |
| 20nt_3- | AAUAGCAAGUUAAAAUAAGGCUAGUCCGUUAUCAACUUGA | |
| 9_005_+_31950044_spCas9 | AAAAGUGGCACCGAGUCGGUGCUsmUsmUsmU | |
| GuideDesign3_ABE_NGA_ | mGsmCsmCsAGAUGAAAGCCAGUCUCGUUUUAGAGCUAGA | 902 |
| 20nt_3- | AAUAGCAAGUUAAAAUAAGGCUAGUCCGUUAUCAACUUGA | |
| 9_005_+_31950044_spCas9 | AAAAGUGGCACCGAGUCGGUGCUsmUsmUsmU | |
| GuideDesign3_ABE_NGA_ | mGsmUsmCsCCCAGCGCCCUUCAAAGGUUUUAGAGCUAGA | 903 |
| 20nt_3- | AAUAGCAAGUUAAAAUAAGGCUAGUCCGUUAUCAACUUGA | |
| 9_005_+_31950277_spCas9 | AAAAGUGGCACCGAGUCGGUGCUsmUsmUsmU | |
| GuideDesign3_ABE_NGA_ | mGsmUsmCsCCCAGCGCCCUUCAAAGGUUUUAGAGCUAGA | 904 |
| 20nt_3- | AAUAGCAAGUUAAAAUAAGGCUAGUCCGUUAUCAACUUGA | |
| 9_005_+_31950277_spCas9 | AAAAGUGGCACCGAGUCGGUGCUsmUsmUsmU | |
| GuideDesign3_ABE_NGA_ | mUsmAsmCsAGAAAGGCAGCUGUGAGGUUUUAGAGCUAGA | 905 |
| 20nt_3- | AAUAGCAAGUUAAAAUAAGGCUAGUCCGUUAUCAACUUGA | |
| 9_005_+_31951335_spCas9 | AAAAGUGGCACCGAGUCGGUGCUsmUsmUsmU | |
| GuideDesign3_ABE_NGA_ | mUsmAsmCsAGAAAGGCAGCUGUGAGGUUUUAGAGCUAGA | 906 |
| 20nt_3- | AAUAGCAAGUUAAAAUAAGGCUAGUCCGUUAUCAACUUGA | |
| 9_005_+_31951335_spCas9 | AAAAGUGGCACCGAGUCGGUGCUsmUsmUsmU | |
| GuideDesign3_ABE_NGC_ | mUsmCsmGsCUUACCUCCAGACAAGAGUUUUAGAGCUAGA | 907 |
| 20nt_3-9_008- | AAUAGCAAGUUAAAAUAAGGCUAGUCCGUUAUCAACUUGA | |
| 31946270_spCas9 | AAAAGUGGCACCGAGUCGGUGCUsmUsmUsmU | |
| GuideDesign3_ABE_NGC_ | mUsmCsmGsCUUACCUCCAGACAAGAGUUUUAGAGCUAGA | 908 |
| 20nt_3-9_008- | AAUAGCAAGUUAAAAUAAGGCUAGUCCGUUAUCAACUUGA | |
| 31946270_spCas9 | AAAAGUGGCACCGAGUCGGUGCUsmUsmUsmU | |
| GuideDesign3_ABE_NGC_ | mUsmCsmAsCCUCCGUUGUCACAGAUGUUUUAGAGCUAGA | 909 |
| 20nt_3-9_008_- | AAUAGCAAGUUAAAAUAAGGCUAGUCCGUUAUCAACUUGA | |
| 31947173_spCas9 | AAAAGUGGCACCGAGUCGGUGCUsmUsmUsmU | |
| GuideDesign3_ABE_NGC_ | mUsmCsmAsCCUCCGUUGUCACAGAUGUUUUAGAGCUAGA | 910 |
| 20nt_3-9_008_- | AAUAGCAAGUUAAAAUAAGGCUAGUCCGUUAUCAACUUGA | |
| 31947173_spCas9 | AAAAGUGGCACCGAGUCGGUGCUsmUsmUsmU | |
| GuideDesign3_ABE_NGC_ | mCsmUsmUsACAAUGACUGAGAUCUUGUUUUAGAGCUAGA | 911 |
| 20nt_3-9_008- | AAUAGCAAGUUAAAAUAAGGCUAGUCCGUUAUCAACUUGA | |
| 31950129_spCas9 | AAAAGUGGCACCGAGUCGGUGCUsmUsmUsmU | |
| GuideDesign3_ABE_NGC_ | mCsmUsmUsACAAUGACUGAGAUCUUGUUUUAGAGCUAGA | 912 |
| 20nt_3-9_008_- | AAUAGCAAGUUAAAAUAAGGCUAGUCCGUUAUCAACUUGA | |
| 31950129_spCas9 | AAAAGUGGCACCGAGUCGGUGCUsmUsmUsmU | |
| GuideDesign3_ABE_NGC_ | mAsmCsmGsCUCUCACCUGAUAGUCUGUUUUAGAGCUAGA | 913 |
| 20nt_3-9_008_- | AAUAGCAAGUUAAAAUAAGGCUAGUCCGUUAUCAACUUGA | |
| 31950759_spCas9 | AAAAGUGGCACCGAGUCGGUGCUsmUsmUsmU | |
| GuideDesign3_ABE_NGC_ | mAsmCsmGsCUCUCACCUGAUAGUCUGUUUUAGAGCUAGA | 914 |
| 20nt_3-9_008- | AAUAGCAAGUUAAAAUAAGGCUAGUCCGUUAUCAACUUGA | |
| 31950759_spCas9 | AAAAGUGGCACCGAGUCGGUGCUsmUsmUsmU | |
| GuideDesign3_ABE_NGC_ | mCsmUsmGsCAGGAUUGCACAACAUGGUUUUAGAGCUAGA | 915 |
| 20nt_3- | AAUAGCAAGUUAAAAUAAGGCUAGUCCGUUAUCAACUUGA | |
| 9_008_+_31949236_spCas9 | AAAAGUGGCACCGAGUCGGUGCUsmUsmUsmU | |
| GuideDesign3_ABE_NGC_ | mCsmUsmGsCAGGAUUGCACAACAUGGUUUUAGAGCUAGA | 916 |
| 20nt_3- | AAUAGCAAGUUAAAAUAAGGCUAGUCCGUUAUCAACUUGA | |
| 9_008_+_31949236_spCas9 | AAAAGUGGCACCGAGUCGGUGCUsmUsmUsmU | |
| GuideDesign3_ABE_NGC_ | mAsmUsmAsGGCCCAUUUGUCUCCCCGUUUUAGAGCUAGA | 917 |
| 20nt_3- | AAUAGCAAGUUAAAAUAAGGCUAGUCCGUUAUCAACUUGA | |
| 9_008_+_31950863_spCas9 | AAAAGUGGCACCGAGUCGGUGCUsmUsmUsmU | |
| GuideDesign3_ABE_NGC_ | mAsmUsmAsGGCCCAUUUGUCUCCCCGUUUUAGAGCUAGA | 918 |
| 20nt_3- | AAUAGCAAGUUAAAAUAAGGCUAGUCCGUUAUCAACUUGA | |
| 9_008_+_31950863_spCas9 | AAAAGUGGCACCGAGUCGGUGCUsmUsmUsmU | |
| GuideDesign3_ABE_NGC_ | mCsmUsmCsUGCAGGUUGGUGUAAUCGUUUUAGAGCUAGA | 919 |
| 20nt_3- | AAUAGCAAGUUAAAAUAAGGCUAGUCCGUUAUCAACUUGA | |
| 9_008_+_31951866_spCas9 | AAAAGUGGCACCGAGUCGGUGCUsmUsmUsmU | |
| GuideDesign3_ABE_NGC_ | mCsmUsmCsUGCAGGUUGGUGUAAUCGUUUUAGAGCUAGA | 920 |
| 20nt_3- | AAUAGCAAGUUAAAAUAAGGCUAGUCCGUUAUCAACUUGA | |
| 9_008_+_31951866_spCas9 | AAAAGUGGCACCGAGUCGGUGCUsmUsmUsmU | |
| GuideDesign3_ABE_NGG_ | mCsmUsmCsGCUUACCUCCAGACAAGGUUUUAGAGCUAGA | 921 |
| 20nt_3-9_002- | AAUAGCAAGUUAAAAUAAGGCUAGUCCGUUAUCAACUUGA | |
| 31946271_spCas9 | AAAAGUGGCACCGAGUCGGUGCUsmUsmUsmU | |
| Gu ideDesign3_ABE_NGG_ | mCsmUsmCsGCUUACCUCCAGACAAGGUUUUAGAGCUAGA | 922 |
| 20nt_3-9_002- | AAUAGCAAGUUAAAAUAAGGCUAGUCCGUUAUCAACUUGA | |
| 31946271_spCas9 | AAAAGUGGCACCGAGUCGGUGCUsmUsmUsmU | |
| GuideDesign3_ABE_NGG_ | mCsmAsmAsAGGUCACCUUGGCAGGAGUUUUAGAGCUAGA | 923 |
| 20nt_3-9_002- | AAUAGCAAGUUAAAAUAAGGCUAGUCCGUUAUCAACUUGA | |
| 31947508_spCas9 | AAAAGUGGCACCGAGUCGGUGCUsmUsmUsmU | |
| GuideDesign3_ABE_NGG_ | mCsmAsmAsAGGUCACCUUGGCAGGAGUUUUAGAGCUAGA | 924 |
| 20nt_3-9_002- | AAUAGCAAGUUAAAAUAAGGCUAGUCCGUUAUCAACUUGA | |
| 31947508_spCas9 | AAAAGUGGCACCGAGUCGGUGCUsmUsmUsmU | |
| GuideDesign3_ABE_NGG_ | mCsmCsmUsUCUGACCAUCAGUCAUGGUUUUAGAGCUAGA | 925 |
| 20nt_3-9_002- | AAUAGCAAGUUAAAAUAAGGCUAGUCCGUUAUCAACUUGA | |
| 31948947_spCas9 | AAAAGUGGCACCGAGUCGGUGCUsmUsmUsmU | |
| GuideDesign3_ABE_NGG_ | mCsmCsmUsUCUGACCAUCAGUCAUGGUUUUAGAGCUAGA | 926 |
| 20nt_3-9_002- | AAUAGCAAGUUAAAAUAAGGCUAGUCCGUUAUCAACUUGA | |
| 31948947_spCas9 | AAAAGUGGCACCGAGUCGGUGCUsmUsmUsmU | |
| GuideDesign3_ABE_NGG_ | mUsmCsmAsCCCAGAUAAUCCUCCCUGUUUUAGAGCUAGA | 927 |
| 20nt_3-9_002- | AAUAGCAAGUUAAAAUAAGGCUAGUCCGUUAUCAACUUGA | |
| 31949325_spCas9 | AAAAGUGGCACCGAGUCGGUGCUsmUsmUsmU | |
| GuideDesign3_ABE_NGG_ | mUsmCsmAsCCCAGAUAAUCCUCCCUGUUUUAGAGCUAGA | 928 |
| 20nt_3-9_002- | AAUAGCAAGUUAAAAUAAGGCUAGUCCGUUAUCAACUUGA | |
| 31949325_spCas9 | AAAAGUGGCACCGAGUCGGUGCUsmUsmUsmU | |
| GuideDesign3_ABE_NGG_ | mGsmCsmUSUACAAUGACUGAGAUCUGUUUUAGAGCUAGA | 929 |
| 20nt_3-9_002- | AAUAGCAAGUUAAAAUAAGGCUAGUCCGUUAUCAACUUGA | |
| 31950130_spCas9 | AAAAGUGGCACCGAGUCGGUGCUsmUsmUsmU | |
| GuideDesign3_ABE_NGG_ | mGsmCsmUsUACAAUGACUGAGAUCUGUUUUAGAGCUAGA | 930 |
| 20nt_3-9_002- | AAUAGCAAGUUAAAAUAAGGCUAGUCCGUUAUCAACUUGA | |
| 31950130_spCas9 | AAAAGUGGCACCGAGUCGGUGCUsmUsmUsmU | |
| GuideDesign3_ABE_NGG_ | mCsmUsmUsACUUUGUUGCUGGCAAGGUUUUAGAGCUAGA | 931 |
| 20nt_3-9_002- | AAUAGCAAGUUAAAAUAAGGCUAGUCCGUUAUCAACUUGA | |
| 31950926_spCas9 | AAAAGUGGCACCGAGUCGGUGCUsmUsmUsmU | |
| GuideDesign3_ABE_NGG_ | mCsmUsmUsACUUUGUUGCUGGCAAGGUUUUAGAGCUAGA | 932 |
| 20nt_3-9_002- | AAUAGCAAGUUAAAAUAAGGCUAGUCCGUUAUCAACUUGA | |
| 31950926_spCas9 | AAAAGUGGCACCGAGUCGGUGCUsmUsmUsmU | |
| GuideDesign3_ABE_NGG_ | mUsmCsmUsCACCUCUGCAAGUAUUGGUUUUAGAGCUAGA | 933 |
| 20nt_3-9_002- | AAUAGCAAGUUAAAAUAAGGCUAGUCCGUUAUCAACUUGA | |
| 31951456_spCas9 | AAAAGUGGCACCGAGUCGGUGCUsmUsmUsmU | |
| GuideDesign3_ABE_NGG_ | mUsmCsmUsCACCUCUGCAAGUAUUGGUUUUAGAGCUAGA | 934 |
| 20nt_3-9_002- | AAUAGCAAGUUAAAAUAAGGCUAGUCCGUUAUCAACUUGA | |
| 31951456_spCas9 | AAAAGUGGCACCGAGUCGGUGCUsmUsmUsmU | |
| GuideDesign3_ABE_NGG_ | mCsmAsmCsCCACAGGUGGCAAGUUAGUUUUAGAGCUAGA | 935 |
| 20nt_3- | AAUAGCAAGUUAAAAUAAGGCUAGUCCGUUAUCAACUUGA | |
| 9_002_+_31948364_spCas9 | AAAAGUGGCACCGAGUCGGUGCUsmUsmUsmU | |
| GuideDesign3_ABE_NGG_ | mCsmAsmCsCCACAGGUGGCAAGUUAGUUUUAGAGCUAGA | 936 |
| 20nt_3- | AAUAGCAAGUUAAAAUAAGGCUAGUCCGUUAUCAACUUGA | |
| 9_002_+_31948364_spCas9 | AAAAGUGGCACCGAGUCGGUGCUsmUsmUsmU | |
| GuideDesign3_ABE_NGG_ | mAsmCsmAsGACCACAAGUUGAAGUCGUUUUAGAGCUAGA | 937 |
| 20nt_3- | AAUAGCAAGUUAAAAUAAGGCUAGUCCGUUAUCAACUUGA | |
| 9_002_+_31948825_spCas9 | AAAAGUGGCACCGAGUCGGUGCUsmUsmUsmU | |
| GuideDesign3_ABE_NGG_ | mAsmCsmAsGACCACAAGUUGAAGUCGUUUUAGAGCUAGA | 938 |
| 20nt_3- | AAUAGCAAGUUAAAAUAAGGCUAGUCCGUUAUCAACUUGA | |
| 9_002_+_31948825_spCas9 | AAAAGUGGCACCGAGUCGGUGCUsmUsmUsmU | |
| GuideDesign3_ABE_NGG_ | mUsmCsmUsGCAGGAUUGCACAACAUGUUUUAGAGCUAGA | 939 |
| 20nt_3- | AAUAGCAAGUUAAAAUAAGGCUAGUCCGUUAUCAACUUGA | |
| 9_002_+_31949235_spCas9 | AAAAGUGGCACCGAGUCGGUGCUsmUsmUsmU | |
| GuideDesign3_ABE_NGG_ | mUsmCsmUsGCAGGAUUGCACAACAUGUUUUAGAGCUAGA | 940 |
| 20nt_3- | AAUAGCAAGUUAAAAUAAGGCUAGUCCGUUAUCAACUUGA | |
| 9_002_+_31949235_spCas9 | AAAAGUGGCACCGAGUCGGUGCUsmUsmUsmU | |
| GuideDesign3_ABE_NGG_ | mGsmCsmAsGGAUUGCACAACAUGGGGUUUUAGAGCUAGA | 941 |
| 20nt_3- | AAUAGCAAGUUAAAAUAAGGCUAGUCCGUUAUCAACUUGA | |
| 9_002_+_31949238_spCas9 | AAAAGUGGCACCGAGUCGGUGCUsmUsmUsmU | |
| GuideDesign3_ABE_NGG_ | mGsmCsmAsGGAUUGCACAACAUGGGGUUUUAGAGCUAGA | 942 |
| 20nt_3- | AAUAGCAAGUUAAAAUAAGGCUAGUCCGUUAUCAACUUGA | |
| 9_002_+_31949238_spCas9 | AAAAGUGGCACCGAGUCGGUGCUsmUsmUsmU | |
| GuideDesign3_ABE_NGG_ | mCsmCsmCsUCAGAUGUCUAUGUGUUGUUUUAGAGCUAGA | 943 |
| 20nt_3- | AAUAGCAAGUUAAAAUAAGGCUAGUCCGUUAUCAACUUGA | |
| 9_002_+_31949412_spCas9 | AAAAGUGGCACCGAGUCGGUGCUsmUsmUsmU | |
| GuideDesign3_ABE_NGG_ | mCsmCsmCsUCAGAUGUCUAUGUGUUGUUUUAGAGCUAGA | 944 |
| 20nt_3- | AAUAGCAAGUUAAAAUAAGGCUAGUCCGUUAUCAACUUGA | |
| 9_002_+_31949412_spCas9 | AAAAGUGGCACCGAGUCGGUGCUsmUsmUsmU | |
| GuideDesign3_ABE_NGG_ | mCsmCsmUsCAGAUGUCUAUGUGUUUGUUUUAGAGCUAGA | 945 |
| 20nt_3- | AAUAGCAAGUUAAAAUAAGGCUAGUCCGUUAUCAACUUGA | |
| 9_002_+_31949413_spCas9 | AAAAGUGGCACCGAGUCGGUGCUsmUsmUsmU | |
| GuideDesign3_ABE_NGG_ | mCsmCsmUsCAGAUGUCUAUGUGUUUGUUUUAGAGCUAGA | 946 |
| 20nt_3- | AAUAGCAAGUUAAAAUAAGGCUAGUCCGUUAUCAACUUGA | |
| 9_002_+_31949413_spCas9 | AAAAGUGGCACCGAGUCGGUGCUsmUsmUsmU | |
| GuideDesign3_ABE_NGG_ | mCsmUsmCsAGAUGUCUAUGUGUUUGGUUUUAGAGCUAGA | 947 |
| 20nt_3- | AAUAGCAAGUUAAAAUAAGGCUAGUCCGUUAUCAACUUGA | |
| 9_002_+_31949414_spCas9 | AAAAGUGGCACCGAGUCGGUGCUsmUsmUsmU | |
| GuideDesign3_ABE_NGG_ | mCsmUsmCsAGAUGUCUAUGUGUUUGGUUUUAGAGCUAGA | 948 |
| 20nt_3- | AAUAGCAAGUUAAAAUAAGGCUAGUCCGUUAUCAACUUGA | |
| 9_002_+_31949414_spCas9 | AAAAGUGGCACCGAGUCGGUGCUsmUsmUsmU | |
| GuideDesign3_ABE_NGG_ | mGsmUsmGsUCCCCAGCGCCCUUCAAGUUUUAGAGCUAGA | 949 |
| 20nt_3- | AAUAGCAAGUUAAAAUAAGGCUAGUCCGUUAUCAACUUGA | |
| 9_002_+_31950275_spCas9 | AAAAGUGGCACCGAGUCGGUGCUsmUsmUsmU | |
| GuideDesign3_ABE_NGG_ | mGsmUsmGsUCCCCAGCGCCCUUCAAGUUUUAGAGCUAGA | 950 |
| 20nt_3- | AAUAGCAAGUUAAAAUAAGGCUAGUCCGUUAUCAACUUGA | |
| 9_002_+_31950275_spCas9 | AAAAGUGGCACCGAGUCGGUGCUsmUsmUsmU | |
| GuideDesign3_ABE_NGG_ | mUsmGsmUsCCCCAGCGCCCUUCAAAGUUUUAGAGCUAGA | 951 |
| 20nt_3- | AAUAGCAAGUUAAAAUAAGGCUAGUCCGUUAUCAACUUGA | |
| 9_002_+_31950276_spCas9 | AAAAGUGGCACCGAGUCGGUGCUsmUsmUsmU | |
| GuideDesign3_ABE_NGG_ | mUsmGsmUsCCCCAGCGCCCUUCAAAGUUUUAGAGCUAGA | 952 |
| 20nt_3- | AAUAGCAAGUUAAAAUAAGGCUAGUCCGUUAUCAACUUGA | |
| 9_002_+_31950276_spCas9 | AAAAGUGGCACCGAGUCGGUGCUsmUsmUsmU | |
| GuideDesign3_ABE_NGG_ | mCsmUsmAsCUUCAGGAGGGGAGAAGGUUUUAGAGCUAGA | 953 |
| 20nt_3- | AAUAGCAAGUUAAAAUAAGGCUAGUCCGUUAUCAACUUGA | |
| 9_002_+_31950609_spCas9 | AAAAGUGGCACCGAGUCGGUGCUsmUsmUsmU | |
| GuideDesign3_ABE_NGG_ | mCsmUsmAsCUUCAGGAGGGGAGAAGGUUUUAGAGCUAGA | 954 |
| 20nt_3- | AAUAGCAAGUUAAAAUAAGGCUAGUCCGUUAUCAACUUGA | |
| 9_002_+_31950609_spCas9 | AAAAGUGGCACCGAGUCGGUGCUsmUsmUsmU | |
| GuideDesign3_ABE_NGG_ | mUsmAsmCsUUCAGGAGGGGAGAAGCGUUUUAGAGCUAGA | 955 |
| 20nt_3- | AAUAGCAAGUUAAAAUAAGGCUAGUCCGUUAUCAACUUGA | |
| 9_002_+_31950610_spCas9 | AAAAGUGGCACCGAGUCGGUGCUsmUsmUsmU | |
| GuideDesign3_ABE_NGG_ | mUsmAsmCsUUCAGGAGGGGAGAAGCGUUUUAGAGCUAGA | 956 |
| 20nt_3- | AAUAGCAAGUUAAAAUAAGGCUAGUCCGUUAUCAACUUGA | |
| 9_002_+_31950610_spCas9 | AAAAGUGGCACCGAGUCGGUGCUsmUsmUsmU | |
| GuideDesign3_ABE_NGG_ | mCsmUsmCsCUUGCAGGUGAUUCUGGGUUUUAGAGCUAGA | 957 |
| 20nt_3- | AAUAGCAAGUUAAAAUAAGGCUAGUCCGUUAUCAACUUGA | |
| 9_002_+_31951544_spCas9 | AAAAGUGGCACCGAGUCGGUGCUsmUsmUsmU | |
| GuideDesign3_ABE_NGG_ | mCsmUsmCsCUUGCAGGUGAUUCUGGGUUUUAGAGCUAGA | 958 |
| 20nt_3- | AAUAGCAAGUUAAAAUAAGGCUAGUCCGUUAUCAACUUGA | |
| 9_002_+_31951544_spCas9 | AAAAGUGGCACCGAGUCGGUGCUsmUsmUsmU | |
| GuideDesign3_ABE_NGG_ | mUsmGsmCsAGGUUGGUGUAAUCAGCGUUUUAGAGCUAGA | 959 |
| 20nt_3- | AAUAGCAAGUUAAAAUAAGGCUAGUCCGUUAUCAACUUGA | |
| 9_002_+_31951869_spCas9 | AAAAGUGGCACCGAGUCGGUGCUsmUsmUsmU | |
| GuideDesign3_ABE_NGG_ | mUsmGsmCsAGGUUGGUGUAAUCAGCGUUUUAGAGCUAGA | 960 |
| 20nt_3- | AAUAGCAAGUUAAAAUAAGGCUAGUCCGUUAUCAACUUGA | |
| 9_002_+_31951869_spCas9 | AAAAGUGGCACCGAGUCGGUGCUsmUsmUsmU | |
| GuideDesign3_ABE_NGG_ | mGsmCsmAsGGUUGGUGUAAUCAGCUGUUUUAGAGCUAGA | 961 |
| 20nt_3- | AAUAGCAAGUUAAAAUAAGGCUAGUCCGUUAUCAACUUGA | |
| 9_002_+_31951870_spCas9 | AAAAGUGGCACCGAGUCGGUGCUsmUsmUsmU | |
| GuideDesign3_ABE_NGG_ | mGsmCsmAsGGUUGGUGUAAUCAGCUGUUUUAGAGCUAGA | 962 |
| 20nt_3- | AAUAGCAAGUUAAAAUAAGGCUAGUCCGUUAUCAACUUGA | |
| 9_002_+_31951870_spCas9 | AAAAGUGGCACCGAGUCGGUGCUsmUsmUsmU | |
| GuideDesign3_ABE_NNGRR | mUsmCsmCsCUUCUGACCAUCAGUCAUGUUUUAGUACUCU | 963 |
| 21nt_5-14_011_- | GUAAUGAAAAUUACAGAAUCUACUAAAACAAGGCAAAAUG | |
| 31948945_saCas9 | CCGUGUUUAUCUCGUCAACUUGUUGGCGAGAUsmUsmUsm | |
| | U | |
| GuideDesign3_ABE_NNGRR | mUsmCsmCsCUUCUGACCAUCAGUCAUGUUUUAGUACUCU | 964 |
| 21nt_5-14_011_- | GUAAUGAAAAUUACAGAAUCUACUAAAACAAGGCAAAAUG | |
| 31948945_saCas9 | CCGUGUUUAUCUCGUCAACUUGUUGGCGAGAUsmUsmUsm | |
| | U | |
| GuideDesign3_ABE_NNGRR | mAsmCsmUsCACCCAGAUAAUCCUCCCGUUUUAGUACUCU | 965 |
| 21nt_5-14_011- | GUAAUGAAAAUUACAGAAUCUACUAAAACAAGGCAAAAUG | |
| 31949323_saCas9 | CCGUGUUUAUCUCGUCAACUUGUUGGCGAGAUsmUsmUsm | |
| | U | |
| GuideDesign3_ABE_NNGRR | mAsmCsmUsCACCCAGAUAAUCCUCCCGUUUUAGUACUCU | 966 |
| 21nt_5-14 011- | GUAAUGAAAAUUACAGAAUCUACUAAAACAAGGCAAAAUG | |
| 31949323_saCas9 | CCGUGUUUAUCUCGUCAACUUGUUGGCGAGAUsmUsmUsm | |
| | U | |
| GuideDesign3_ABE_NNGRR | mCsmCsmUsUACCUACGCUGACCUUGAGUUUUAGUACUCU | 967 |
| 21nt_5-14_011 | GUAAUGAAAAUUACAGAAUCUACUAAAACAAGGCAAAAUG | |
| 31950382_saCas9 | CCGUGUUUAUCUCGUCAACUUGUUGGCGAGAUsmUsmUsm | |
| | U | |
| GuideDesign3_ABE_NNGRR | mCsmCsmUsUACCUACGCUGACCUUGAGUUUUAGUACUCU | 968 |
| 21nt_5-14 011- | GUAAUGAAAAUUACAGAAUCUACUAAAACAAGGCAAAAUG | |
| 31950382_saCas9 | CCGUGUUUAUCUCGUCAACUUGUUGGCGAGAUsmUsmUsm | |
| | U | |
| GuideDesign3_ABE_NNGRR | mUsmUsmCsUCUCACCUCUGCAAGUAUGUUUUAGUACUCU | 969 |
| 21nt_5-14_011- | GUAAUGAAAAUUACAGAAUCUACUAAAACAAGGCAAAAUG | |
| 31951455_saCas9 | CCGUGUUUAUCUCGUCAACUUGUUGGCGAGAUsmUsmUsm | |
| | U | |
| GuideDesign3_ABE_NNGRR | mUsmUsmCsUCUCACCUCUGCAAGUAUGUUUUAGUACUCU | 970 |
| 21nt_5-14 011- | GUAAUGAAAAUUACAGAAUCUACUAAAACAAGGCAAAAUG | |
| 31951455_saCas9 | CCGUGUUUAUCUCGUCAACUUGUUGGCGAGAUsmUsmUsm | |
| | U | |
| GuideDesign3_ABE_NNGRR | mUsmCsmCsCUCAGAUGUCUAUGUGUUGUUUUAGUACUCU | 971 |
| 21nt_5- | GUAAUGAAAAUUACAGAAUCUACUAAAACAAGGCAAAAUG | |
| 14 011_+_31949411_saCas9 | CCGUGUUUAUCUCGUCAACUUGUUGGCGAGAUsmUsmUsm | |
| | U | |
| GuideDesign3_ABE_NNGRR | mUsmCsmCsCUCAGAUGUCUAUGUGUUGUUUUAGUACUCU | 972 |
| 21nt_5- | GUAAUGAAAAUUACAGAAUCUACUAAAACAAGGCAAAAUG | |
| 14 011_+_31949411_saCas9 | CCGUGUUUAUCUCGUCAACUUGUUGGCGAGAUsmUsmUsm | |
| | U | |
| GuideDesign3_ABE_NNGRR | mCsmUsmGsCCAGAUGAAAGCCAGUCUGUUUUAGUACUCU | 973 |
| 21nt_5- | GUAAUGAAAAUUACAGAAUCUACUAAAACAAGGCAAAAUG | |
| 14 011_+_31950042_saCas9 | CCGUGUUUAUCUCGUCAACUUGUUGGCGAGAUsmUsmUsm | |
| | U | |
| GuideDesign3_ABE_NNGRR | mCsmUsmGsCCAGAUGAAAGCCAGUCUGUUUUAGUACUCU | 974 |
| 21nt_5- | GUAAUGAAAAUUACAGAAUCUACUAAAACAAGGCAAAAUG | |
| 14 011_+_31950042_saCas9 | CCGUGUUUAUCUCGUCAACUUGUUGGCGAGAUsmUsmUsm | |
| | U | |
| GuideDesign3_ABE_NNNRRT_ | mAsmUsmGsCUUCUCACCUCCGUUGUCGUUUUAGUACUCU | 975 |
| 21nt_5-14_014_- | GUAAUGAAAAUUACAGAAUCUACUAAAACAAGGCAAAAUG | |
| 31947176_saCas9 | CCGUGUUUAUCUCGUCAACUUGUUGGCGAGAUsmUsmUsm | |
| | U | |
| GuideDesign3_ABE_NNNRRT_ | mAsmUsmGsCUUCUCACCUCCGUUGUCGUUUUAGUACUCU | 976 |
| 21nt_5-14_014- | GUAAUGAAAAUUACAGAAUCUACUAAAACAAGGCAAAAUG | |
| 31947176_saCas9 | CCGUGUUUAUCUCGUCAACUUGUUGGCGAGAUsmUsmUsm | |
| | U | |
| GuideDesign3_ABE_NNNRRT_ | mAsmGsmGsAGGAUUCCACCUUCUCAAGUUUUAGUACUCU | 977 |
| 21nt_5-14_014- | GUAAUGAAAAUUACAGAAUCUACUAAAACAAGGCAAAAUG | |
| 31948067_saCas9 | CCGUGUUUAUCUCGUCAACUUGUUGGCGAGAUsmUsmUsm | |
| | U | |
| GuideDesign3_ABE_NNNRRT_ | mAsmGsmGsAGGAUUCCACCUUCUCAAGUUUUAGUACUCU | 978 |
| 21nt_5-14_014- | GUAAUGAAAAUUACAGAAUCUACUAAAACAAGGCAAAAUG | |
| 31948067_saCas9 | CCGUGUUUAUCUCGUCAACUUGUUGGCGAGAUsmUsmUsm | |
| | U | |
| GuideDesign3_ABE_NNNRRT_ | mCsmCsmCsUAACCUCUGACCUUCAUAGUUUUAGUACUCU | 979 |
| 21nt_5-14_014- | GUAAUGAAAAUUACAGAAUCUACUAAAACAAGGCAAAAUG | |
| 31948499_saCas9 | CCGUGUUUAUCUCGUCAACUUGUUGGCGAGAUsmUsmUsm | |
| | U | |
| GuideDesign3_ABE_NNNRRT_ | mCsmCsmCsUAACCUCUGACCUUCAUAGUUUUAGUACUCU | 980 |
| 21nt_5-14_014- | GUAAUGAAAAUUACAGAAUCUACUAAAACAAGGCAAAAUG | |
| 31948499_saCas9 | CCGUGUUUAUCUCGUCAACUUGUUGGCGAGAUsmUsmUsm | |
| | U | |
| GuideDesign3_ABE_NNNRRT_ | mCsmUsmUsCUGACCAUCAGUCAUGAGGUUUUAGUACUCU | 981 |
| 21nt_5-14_014- | GUAAUGAAAAUUACAGAAUCUACUAAAACAAGGCAAAAUG | |
| 31948942_saCas9 | CCGUGUUUAUCUCGUCAACUUGUUGGCGAGAUsmUsmUsm | |
| | U | |
| GuideDesign3_ABE_NNNRRT_ | mCsmUsmUsCUGACCAUCAGUCAUGAGGUUUUAGUACUCU | 982 |
| 21nt_5-14_014- | GUAAUGAAAAUUACAGAAUCUACUAAAACAAGGCAAAAUG | |
| 31948942_saCas9 | CCGUGUUUAUCUCGUCAACUUGUUGGCGAGAUsmUsmUsm | |
| | U | |
| GuideDesign3_ABE_NNNRRT_ | mCsmUsmUsGUAUCUCCCUACCGAUCAGUUUUAGUACUCU | 983 |
| 21nt_5-14_014- | GUAAUGAAAAUUACAGAAUCUACUAAAACAAGGCAAAAUG | |
| 31949545_saCas9 | CCGUGUUUAUCUCGUCAACUUGUUGGCGAGAUsmUsmUsm | |
| | U | |
| GuideDesign3_ABE_NNNRRT_ | mCsmUsmUsGUAUCUCCCUACCGAUCAGUUUUAGUACUCU | 984 |
| 21nt_5-14_014- | GUAAUGAAAAUUACAGAAUCUACUAAAACAAGGCAAAAUG | |
| 31949545_saCas9 | CCGUGUUUAUCUCGUCAACUUGUUGGCGAGAUsmUsmUsm | |
| | U | |
| GuideDesign3_ABE_NNNRRT_ | mGsmGsmAsUUCUGUGCUUACAAUGACGUUUUAGUACUCU | 985 |
| 21nt_5-14_014- | GUAAUGAAAAUUACAGAAUCUACUAAAACAAGGCAAAAUG | |
| 31950135_saCas9 | CCGUGUUUAUCUCGUCAACUUGUUGGCGAGAUsmUsmUsm | |
| | U | |
| GuideDesign3_ABE_NNNRRT_ | mGsmGsmAsUUCUGUGCUUACAAUGACGUUUUAGUACUCU | 986 |
| 21nt_5-14_014- | GUAAUGAAAAUUACAGAAUCUACUAAAACAAGGCAAAAUG | |
| 31950135_saCas9 | CCGUGUUUAUCUCGUCAACUUGUUGGCGAGAUsmUsmUsm | |
| | U | |
| GuideDesign3_ABE_NNNRRT_ | mUsmGsmCsAUCCUUACCUACGCUGACGUUUUAGUACUCU | 987 |
| 21nt_5-14_014- | GUAAUGAAAAUUACAGAAUCUACUAAAACAAGGCAAAAUG | |
| 31950387_saCas9 | CCGUGUUUAUCUCGUCAACUUGUUGGCGAGAUsmUsmUsm | |
| | U | |
| GuideDesign3_ABE_NNNRRT_ | mUsmGsmCsAUCCUUACCUACGCUGACGUUUUAGUACUCU | 988 |
| 21nt_5-14_014- | GUAAUGAAAAUUACAGAAUCUACUAAAACAAGGCAAAAUG | |
| 31950387_saCas9 | CCGUGUUUAUCUCGUCAACUUGUUGGCGAGAUsmUsmUsm | |
| | U | |
| GuideDesign3_ABE_NNNRRT_ | mUsmGsmUsCUUACUUUGUUGCUGGCAGUUUUAGUACUCU | 989 |
| 21nt_5-14_014- | GUAAUGAAAAUUACAGAAUCUACUAAAACAAGGCAAAAUG | |
| 31950925_saCas9 | CCGUGUUUAUCUCGUCAACUUGUUGGCGAGAUsmUsmUsm | |
| | U | |
| GuideDesign3_ABE_NNNRRT_ | mUsmGsmUsCUUACUUUGUUGCUGGCAGUUUUAGUACUCU | 990 |
| 21nt_5-14_014- | GUAAUGAAAAUUACAGAAUCUACUAAAACAAGGCAAAAUG | |
| 31950925_saCas9 | CCGUGUUUAUCUCGUCAACUUGUUGGCGAGAUsmUsmUsm | |
| | U | |
| GuideDesign3_ABE_NNNRRT_ | mGsmUsmAsUGUCUUACUUUGUUGCUGGUUUUAGUACUCU | 991 |
| 21nt_5-14_014- | GUAAUGAAAAUUACAGAAUCUACUAAAACAAGGCAAAAUG | |
| 31950928_saCas9 | CCGUGUUUAUCUCGUCAACUUGUUGGCGAGAUsmUsmUsm | |
| | U | |
| GuideDesign3_ABE_NNNRRT_ | mGsmUsmAsUGUCUUACUUUGUUGCUGGUUUUAGUACUCU | 992 |
| 21nt_5-14_014- | GUAAUGAAAAUUACAGAAUCUACUAAAACAAGGCAAAAUG | |
| 31950928_saCas9 | CCGUGUUUAUCUCGUCAACUUGUUGGCGAGAUsmUsmUsm | |
| | U | |
| GuideDesign3_ABE_NNNRRT_ | mCsmUsmUsCAGGUGUGACCACCACUCGUUUUAGUACUCU | 993 |
| 21nt_5- | GUAAUGAAAAUUACAGAAUCUACUAAAACAAGGCAAAAUG | |
| 14_014_+_31946366_saCas9 | CCGUGUUUAUCUCGUCAACUUGUUGGCGAGAUsmUsmUsm | |
| | U | |
| GuideDesign3_ABE_NNNRRT_ | mCsmUsmUsCAGGUGUGACCACCACUCGUUUUAGUACUCU | 994 |
| 21nt_5- | GUAAUGAAAAUUACAGAAUCUACUAAAACAAGGCAAAAUG | |
| 14_014_+_31946366_saCas9 | CCGUGUUUAUCUCGUCAACUUGUUGGCGAGAUsmUsmUsm | |
| | U | |
| GuideDesign3_ABE_NNNRRT_ | mCsmCsmCsUCUCCACAGACCACAAGUGUUUUAGUACUCU | 995 |
| 21nt_5- | GUAAUGAAAAUUACAGAAUCUACUAAAACAAGGCAAAAUG | |
| 14_014_+_31948817_saCas9 | CCGUGUUUAUCUCGUCAACUUGUUGGCGAGAUsmUsmUsm | |
| | U | |
| GuideDesign3_ABE_NNNRRT_ | mCsmCsmCsUCUCCACAGACCACAAGUGUUUUAGUACUCU | 996 |
| 21nt_5- | GUAAUGAAAAUUACAGAAUCUACUAAAACAAGGCAAAAUG | |
| 14_014_+_31948817_saCas9 | CCGUGUUUAUCUCGUCAACUUGUUGGCGAGAUsmUsmUsm | |
| | U | |
| GuideDesign3_ABE_NNNRRT_ | mCsmUsmAsCAGAAAGGCAGCUGUGAGGUUUUAGUACUCU | 997 |
| 21nt_5- | GUAAUGAAAAUUACAGAAUCUACUAAAACAAGGCAAAAUG | |
| 14_014_+_31951334_saCas9 | CCGUGUUUAUCUCGUCAACUUGUUGGCGAGAUsmUsmUsm | |
| | U | |
| GuideDesign3_ABE_NNNRRT_ | mCsmUsmAsCAGAAAGGCAGCUGUGAGGUUUUAGUACUCU | 998 |
| 21nt_5- | GUAAUGAAAAUUACAGAAUCUACUAAAACAAGGCAAAAUG | |
| 14_014_+_31951334_saCas9 | CCGUGUUUAUCUCGUCAACUUGUUGGCGAGAUsmUsmUsm | |
| | U | |
| GuideDesign3_CBE_NNGRR | mUsmGsmUsUGUUCCCCUGGGUGCCAGGUUUUAGUACUCU | 999 |
| 21nt_3-12_012- | GUAAUGAAAAUUACAGAAUCUACUAAAACAAGGCAAAAUG | |
| 31947927_saCas9 | CCGUGUUUAUCUCGUCAACUUGUUGGCGAGAUsmUsmUsm | |
| | U | |
| GuideDesign3_CBE_NNGRR | mUsmGsmUsUGUUCCCCUGGGUGCCAGGUUUUAGUACUCU | 1000 |
| 21nt_3-12_012- | GUAAUGAAAAUUACAGAAUCUACUAAAACAAGGCAAAAUG | |
| 31947927_saCas9 | CCGUGUUUAUCUCGUCAACUUGUUGGCGAGAUsmUsmUsm | |
| | U | |
| GuideDesign3_CBE_NNGRR | mGsmAsmCsAUCUGAGGGAUAAAAAGGGUUUUAGUACUCU | 1001 |
| 21nt_3-12_012- | GUAAUGAAAAUUACAGAAUCUACUAAAACAAGGCAAAAUG | |
| 31949397_saCas9 | CCGUGUUUAUCUCGUCAACUUGUUGGCGAGAUsmUsmUsm | |
| | U | |
| GuideDesign3_CBE_NNGRR | mGsmAsmCsAUCUGAGGGAUAAAAAGGGUUUUAGUACUCU | 1002 |
| 21nt_3-12_012- | GUAAUGAAAAUUACAGAAUCUACUAAAACAAGGCAAAAUG | |
| 31949397_saCas9 | CCGUGUUUAUCUCGUCAACUUGUUGGCGAGAUsmUsmUsm | |
| | U | |
| GuideDesign3_CBE_NNGRR | mGsmGsmCsUUUCAUCUGGCAGAGAAGGUUUUAGUACUCU | 1003 |
| 21nt_3-12_012- | GUAAUGAAAAUUACAGAAUCUACUAAAACAAGGCAAAAUG | |
| 31950031_saCas9 | CCGUGUUUAUCUCGUCAACUUGUUGGCGAGAUsmUsmUsm | |
| | U | |
| GuideDesign3_CBE_NNGRR | mGsmGsmCsUUUCAUCUGGCAGAGAAGGUUUUAGUACUCU | 1004 |
| 21nt_3-12_012- | GUAAUGAAAAUUACAGAAUCUACUAAAACAAGGCAAAAUG | |
| 31950031_saCas9 | CCGUGUUUAUCUCGUCAACUUGUUGGCGAGAUsmUsmUsm | |
| | U | |
| GuideDesign3_CBE_NNGRR | mUsmAsmCsACCAACCUGCAGAGGCAGGUUUUAGUACUCU | 1005 |
| 21nt_3-12_012_- | GUAAUGAAAAUUACAGAAUCUACUAAAACAAGGCAAAAUG | |
| 31951856_saCas9 | CCGUGUUUAUCUCGUCAACUUGUUGGCGAGAUsmUsmUsm | |
| | U | |
| GuideDesign3_CBE_NNGRR | mUsmAsmCsACCAACCUGCAGAGGCAGGUUUUAGUACUCU | 1006 |
| 21nt_3-12_012_- | GUAAUGAAAAUUACAGAAUCUACUAAAACAAGGCAAAAUG | |
| 31951856_saCas9 | CCGUGUUUAUCUCGUCAACUUGUUGGCGAGAUsmUsmUsm | |
| | U | |
| GuideDesign3_CBE_NNNRRT_ | mCsmUsmUsACCUCCAGACAAGAGGCCGUUUUAGUACUCU | 1007 |
| 21nt_3-12_015- | GUAAUGAAAAUUACAGAAUCUACUAAAACAAGGCAAAAUG | |
| 31946263_saCas9 | CCGUGUUUAUCUCGUCAACUUGUUGGCGAGAUsmUsmUsm | |
| | U | |
| GuideDesign3_CBE_NNNRRT_ | mCsmUsmUsACCUCCAGACAAGAGGCCGUUUUAGUACUCU | 1008 |
| 21nt_3-12_015_- | GUAAUGAAAAUUACAGAAUCUACUAAAACAAGGCAAAAUG | |
| 31946263_saCas9 | CCGUGUUUAUCUCGUCAACUUGUUGGCGAGAUsmUsmUsm | |
| | U | |
| GuideDesign3_CBE_NNNRRT_ | mUsmGsmGsUCACACCUGAAGAGAAAGGUUUUAGUACUCU | 1009 |
| 21nt_3-12_015_- | GUAAUGAAAAUUACAGAAUCUACUAAAACAAGGCAAAAUG | |
| 31946354_saCas9 | CCGUGUUUAUCUCGUCAACUUGUUGGCGAGAUsmUsmUsm | |
| | U | |
| GuideDesign3_CBE_NNNRRT_ | mUsmGsmGsUCACACCUGAAGAGAAAGGUUUUAGUACUCU | 1010 |
| 21nt_3-12_015_- | GUAAUGAAAAUUACAGAAUCUACUAAAACAAGGCAAAAUG | |
| 31946354_saCas9 | CCGUGUUUAUCUCGUCAACUUGUUGGCGAGAUsmUsmUsm | |
| | U | |
| GuideDesign3_CBE_NNNRRT_ | mGsmGsmAsUUGCUUUGAGAAGGGGGGGUUUUAGUACUCU | 1011 |
| 21nt_3-12_015- | GUAAUGAAAAUUACAGAAUCUACUAAAACAAGGCAAAAUG | |
| 31946985_saCas9 | CCGUGUUUAUCUCGUCAACUUGUUGGCGAGAUsmUsmUsm | |
| | U | |
| GuideDesign3_CBE_NNNRRT_ | mGsmGsmAsUUGCUUUGAGAAGGGGGGGUUUUAGUACUCU | 1012 |
| 21nt_3-12_015- | GUAAUGAAAAUUACAGAAUCUACUAAAACAAGGCAAAAUG | |
| 31946985_saCas9 | CCGUGUUUAUCUCGUCAACUUGUUGGCGAGAUsmUsmUsm | |
| | U | |
| GuideDesign3_CBE_NNNRRT_ | mCsmGsmCsUGCAGAGGUAUGAGACAUGUUUUAGUACUCU | 1013 |
| 21nt_3-12_015- | GUAAUGAAAAUUACAGAAUCUACUAAAACAAGGCAAAAUG | |
| 31947322_saCas9 | CCGUGUUUAUCUCGUCAACUUGUUGGCGAGAUsmUsmUsm | |
| | U | |
| GuideDesign3_CBE_NNNRRT_ | mCsmGsmCsUGCAGAGGUAUGAGACAUGUUUUAGUACUCU | 1014 |
| 21nt_3-12_015_- | GUAAUGAAAAUUACAGAAUCUACUAAAACAAGGCAAAAUG | |
| 31947322_saCas9 | CCGUGUUUAUCUCGUCAACUUGUUGGCGAGAUsmUsmUsm | |
| | U | |
| GuideDesign3_CBE_NNNRRT_ | mGsmGsmAsGUCUGGGAGAGUCAGAAAGUUUUAGUACUCU | 1015 |
| 21nt_3-12_015- | GUAAUGAAAAUUACAGAAUCUACUAAAACAAGGCAAAAUG | |
| 31947719_saCas9 | CCGUGUUUAUCUCGUCAACUUGUUGGCGAGAUsmUsmUsm | |
| | U | |
| GuideDesign3_CBE_NNNRRT_ | mGsmGsmAsGUCUGGGAGAGUCAGAAAGUUUUAGUACUCU | 1016 |
| 21nt_3-12_015- | GUAAUGAAAAUUACAGAAUCUACUAAAACAAGGCAAAAUG | |
| 31947719_saCas9 | CCGUGUUUAUCUCGUCAACUUGUUGGCGAGAUsmUsmUsm | |
| | U | |
| GuideDesign3_CBE_NNNRRT_ | mAsmUsmGsAAGGAGUCUGGGAGAGUCGUUUUAGUACUCU | 1017 |
| 21nt_3-12_015- | GUAAUGAAAAUUACAGAAUCUACUAAAACAAGGCAAAAUG | |
| 31947724_saCas9 | CCGUGUUUAUCUCGUCAACUUGUUGGCGAGAUsmUsmUsm | |
| | U | |
| GuideDesign3_CBE_NNNRRT_ | mAsmUsmGsAAGGAGUCUGGGAGAGUCGUUUUAGUACUCU | 1018 |
| 21nt_3-12_015- | GUAAUGAAAAUUACAGAAUCUACUAAAACAAGGCAAAAUG | |
| 31947724_saCas9 | CCGUGUUUAUCUCGUCAACUUGUUGGCGAGAUsmUsmUsm | |
| | U | |
| GuideDesign3_CBE_NNNRRT_ | mUsmUsmUsGAAGGGCGCUGGGGACACGUUUUAGUACUCU | 1019 |
| 21nt_3-12_015- | GUAAUGAAAAUUACAGAAUCUACUAAAACAAGGCAAAAUG | |
| 31950269_saCas9 | CCGUGUUUAUCUCGUCAACUUGUUGGCGAGAUsmUsmUsm | |
| | U | |
| GuideDesign3_CBE_NNNRRT_ | mUsmUsmUsGAAGGGCGCUGGGGACACGUUUUAGUACUCU | 1020 |
| 21nt_3-12_015- | GUAAUGAAAAUUACAGAAUCUACUAAAACAAGGCAAAAUG | |
| 31950269_saCas9 | CCGUGUUUAUCUCGUCAACUUGUUGGCGAGAUsmUsmUsm | |
| | U | |
| GuideDesign3_CBE_NNNRRT_ | mCsmUsmCsCCCUCCUGAAGUAGGAGAGUUUUAGUACUCU | 1021 |
| 21nt_3-12_015- | GUAAUGAAAAUUACAGAAUCUACUAAAACAAGGCAAAAUG | |
| 31950599_saCas9 | CCGUGUUUAUCUCGUCAACUUGUUGGCGAGAUsmUsmUsm | |
| | U | |
| GuideDesign3_CBE_NNNRRT_ | mCsmUsmCsCCCUCCUGAAGUAGGAGAGUUUUAGUACUCU | 1022 |
| 21nt_3-12_015 | GUAAUGAAAAUUACAGAAUCUACUAAAACAAGGCAAAAUG | |
| 31950599_saCas9 | CCGUGUUUAUCUCGUCAACUUGUUGGCGAGAUsmUsmUsm | |
| | U | |
| GuideDesign3_CBE_NNNRRT_ | mCsmUsmUsACUUUGUUGCUGGCAAGUGUUUUAGUACUCU | 1023 |
| 21nt_3-12_015- | GUAAUGAAAAUUACAGAAUCUACUAAAACAAGGCAAAAUG | |
| 31950922_saCas9 | CCGUGUUUAUCUCGUCAACUUGUUGGCGAGAUsmUsmUsm | |
| | U | |
| GuideDesign3_CBE_NNNRRT_ | mCsmUsmUsACUUUGUUGCUGGCAAGUGUUUUAGUACUCU | 1024 |
| 21nt_3-12_015- | GUAAUGAAAAUUACAGAAUCUACUAAAACAAGGCAAAAUG | |
| 31950922_saCas9 | CCGUGUUUAUCUCGUCAACUUGUUGGCGAGAUsmUsmUsm | |
| | U | |
| GuideDesign10_CBE_NGC_ | mAsmCsmUsCCAUCGGCCAUUCACUUGUUUUAGAGCUAGA | 1025 |
| 20nt_4-9_009- | AAUAGCAAGUUAAAAUAAGGCUAGUCCGUUAUCAACUUGA | |
| 31947141_spCas9 | AAAAGUGGCACCGAGUCGGUGCUsmUsmUsmU | |
| GuideDesign10_CBE_NGC_ | mAsmCsmCsCAAAUUUUGGGGUAUGUGUUUUAGAGCUAGA | 1026 |
| 20nt_4-9_009- | AAUAGCAAGUUAAAAUAAGGCUAGUCCGUUAUCAACUUGA | |
| 31948418_spCas9 | AAAAGUGGCACCGAGUCGGUGCUsmUsmUsmU | |
| GuideDesign10_CBE_NGC_ | mCsmGsmUsGACCCAGUCUGCAUUACGUUUUAGAGCUAGA | 1027 |
| 20nt_4-9_009- | AAUAGCAAGUUAAAAUAAGGCUAGUCCGUUAUCAACUUGA | |
| 31948461_spCas9 | AAAAGUGGCACCGAGUCGGUGCUsmUsmUsmU | |
| GuideDesign10_CBE_NGC_ | mGsmAsmCsCAAAAGACUGUCAGGAAGUUUUAGAGCUAGA | 1028 |
| 20nt_4- | AAUAGCAAGUUAAAAUAAGGCUAGUCCGUUAUCAACUUGA | |
| 9_009_+_31946572_spCas9 | AAAAGUGGCACCGAGUCGGUGCUsmUsmUsmU | |
| GuideDesign10_CBE_NGC_ | mGsmGsmCsCGAUGGAGUGGGCAGACGUUUUAGAGCUAGA | 1029 |
| 20nt_4- | AAUAGCAAGUUAAAAUAAGGCUAGUCCGUUAUCAACUUGA | |
| 9_009_+_31947152_spCas9 | AAAAGUGGCACCGAGUCGGUGCUsmUsmUsmU | |
| GuideDesign10_CBE_NGC_ | mAsmCsmCsCCUCAAGAGGUGGCCGAGUUUUAGAGCUAGA | 1030 |
| 20nt_4- | AAUAGCAAGUUAAAAUAAGGCUAGUCCGUUAUCAACUUGA | |
| 9_009_+_31947758_spCas9 | AAAAGUGGCACCGAGUCGGUGCUsmUsmUsmU | |
| GuideDesign10_CBE_NGC_ | mAsmGsmGsCCCUCCAGGCAGUGUACGUUUUAGAGCUAGA | 1031 |
| 20nt_4- | AAUAGCAAGUUAAAAUAAGGCUAGUCCGUUAUCAACUUGA | |
| 9_009_+_31948862_spCas9 | AAAAGUGGCACCGAGUCGGUGCUsmUsmUsmU | |
| GuideDesign10_CBE_NGC_ | mUsmGsmGsCCAGACUAUCAGGUGAGGUUUUAGAGCUAGA | 1032 |
| 20nt_4- | AAUAGCAAGUUAAAAUAAGGCUAGUCCGUUAUCAACUUGA | |
| 9_009_+_31950757_spCas9 | AAAAGUGGCACCGAGUCGGUGCUsmUsmUsmU | |
| GuideDesign10_CBE_NGC_ | mCsmAsmAsCAAAGUAAGACAUACUUGUUUUAGAGCUAGA | 1033 |
| 20nt_4- | AAUAGCAAGUUAAAAUAAGGCUAGUCCGUUAUCAACUUGA | |
| 9_009_+_31950937_spCas9 | AAAAGUGGCACCGAGUCGGUGCUsmUsmUsmU | |
| GuideDesign10_CBE_NGC_ | mCsmAsmAsAAGCAGGUACCUGCUCAGUUUUAGAGCUAGA | 1034 |
| 20nt_4- | AAUAGCAAGUUAAAAUAAGGCUAGUCCGUUAUCAACUUGA | |
| 9_009_+_31951925_spCas9 | AAAAGUGGCACCGAGUCGGUGCUsmUsmUsmU | |
| GuideDesign10_CBE_NGC_ | mCsmCsmUsCUUUCAAGUGCUGCCCUGUUUUAGAGCUAGA | 1035 |
| 20nt_4- | AAUAGCAAGUUAAAAUAAGGCUAGUCCGUUAUCAACUUGA | |
| 9_009_+_31951966_spCas9 | AAAAGUGGCACCGAGUCGGUGCUsmUsmUsmU | |
| GuideDesign3_CBE_NNGRR | mGsmAsmGsAAACUCCAAGAUGAGGAUGUUUUAGUACUCU | 1036 |
| 21nt_3- | GUAAUGAAAAUUACAGAAUCUACUAAAACAAGGCAAAAUG | |
| 12_012_+_31951994_saCas9 | CCGUGUUUAUCUCGUCAACUUGUUGGCGAGAUsmUsmUsm | |
| | U | |
| GuideDesign3_CBE_NNNRRT_ | mCsmUsmGsCCAUGGUUGCUUGUGGUAGUUUUAGUACUCU | 1037 |
| 21nt_3-12_015- | GUAAUGAAAAUUACAGAAUCUACUAAAACAAGGCAAAAUG | |
| 31950102_saCas9 | CCGUGUUUAUCUCGUCAACUUGUUGGCGAGAUsmUsmUsm | |
| | U | |
| GuideDesign3_CBE_NNNRRT_ | mUsmUsmCsUCCAAGAGGGCCAGGCACGUUUUAGUACUCU | 1038 |
| 21nt_3- | GUAAUGAAAAUUACAGAAUCUACUAAAACAAGGCAAAAUG | |
| 12_015_+_31946459_saCas9 | CCGUGUUUAUCUCGUCAACUUGUUGGCGAGAUsmUsmUsm | |
| | U | |
| GuideDesign3_CBE_NNNRRT_ | mAsmCsmCsCUGUGCAGACACGUACCUGUUUUAGUACUCU | 1039 |
| 21nt_3- | GUAAUGAAAAUUACAGAAUCUACUAAAACAAGGCAAAAUG | |
| 12_015_+_31946513_saCas9 | CCGUGUUUAUCUCGUCAACUUGUUGGCGAGAUsmUsmUsm | |
| | U | |
| GuideDesign3_CBE_NNNRRT_ | mAsmCsmCsAAAAGACUGUCAGGAAGGGUUUUAGUACUCU | 1040 |
| 21nt_3- | GUAAUGAAAAUUACAGAAUCUACUAAAACAAGGCAAAAUG | |
| 12_015_+_31946573_saCas9 | CCGUGUUUAUCUCGUCAACUUGUUGGCGAGAUsmUsmUsm | |
| | U | |
| GuideDesign3_CBE_NNNRRT_ | mAsmCsmCsUGCCAAGUGAAUGGCCGAGUUUUAGUACUCU | 1041 |
| 21nt_3- | GUAAUGAAAAUUACAGAAUCUACUAAAACAAGGCAAAAUG | |
| 12_015_+_31947137_saCas9 | CCGUGUUUAUCUCGUCAACUUGUUGGCGAGAUsmUsmUsm | |
| | U | |
| GuideDesign3_CBE_NNNRRT_ | mAsmGsmAsCAAUGAGCAACAUGUGUUGUUUUAGUACUCU | 1042 |
| 21nt_3- | GUAAUGAAAAUUACAGAAUCUACUAAAACAAGGCAAAAUG | |
| 12_015_+_31949486_saCas9 | CCGUGUUUAUCUCGUCAACUUGUUGGCGAGAUsmUsmUsm | |
| | U | |
| GuideDesign3_CBE_NNNRRT_ | mGsmAsmGsCAACAUGUGUUCAAAGUCGUUUUAGUACUCU | 1043 |
| 21nt_3- | GUAAUGAAAAUUACAGAAUCUACUAAAACAAGGCAAAAUG | |
| 12_015_+_31949493_saCas9 | CCGUGUUUAUCUCGUCAACUUGUUGGCGAGAUsmUsmUsm | |
| | U | |
| GuideDesign3_CBE_NNNRRT_ | mCsmCsmAsCAAGCAACCAUGGCAGGCGUUUUAGUACUCU | 1044 |
| 21nt_3- | GUAAUGAAAAUUACAGAAUCUACUAAAACAAGGCAAAAUG | |
| 12_015_+_31950110_saCas9 | CCGUGUUUAUCUCGUCAACUUGUUGGCGAGAUsmUsmUsm | |
| | U | |
| GuideDesign3_CBE_NNNRRT_ | mCsmAsmAsAAACCAGAAGCGGCAAAAGUUUUAGUACUCU | 1045 |
| 21nt_3- | GUAAUGAAAAUUACAGAAUCUACUAAAACAAGGCAAAAUG | |
| 12_015_+_31951909_saCas9 | CCGUGUUUAUCUCGUCAACUUGUUGGCGAGAUsmUsmUsm | |
| | U | |
| GuideDesign3_CBE_NNNRRT_ | mGsmAsmGsAAACUCCAAGAUGAGGAUGUUUUAGUACUCU | 1046 |
| 21nt_3- | GUAAUGAAAAUUACAGAAUCUACUAAAACAAGGCAAAAUG | |
| 12_015_+_31951994_saCas9 | CCGUGUUUAUCUCGUCAACUUGUUGGCGAGAUsmUsmUsm | |
| | U | |
| GuideDesign10_CBE_NGA_ | mAsmGsmGsGUGCUCCAGGACCCCGUGUUUUAGAGCUAGA | 1047 |
| 20nt_4-9_006- | AAUAGCAAGUUAAAAUAAGGCUAGUCCGUUAUCAACUUGA | |
| 31946539_spCas9 | AAAAGUGGCACCGAGUCGGUGCUsmUsmUsmU | |
| GuideDesign10_CBE_NGA_ | mGsmGsmGsCCAGUAUUCCCCGUUCUGUUUUAGAGCUAGA | 1048 |
| 20nt_4-9_006_- | AAUAGCAAGUUAAAAUAAGGCUAGUCCGUUAUCAACUUGA | |
| 31947033_spCas9 | AAAAGUGGCACCGAGUCGGUGCUsmUsmUsmU | |
| GuideDesign10_CBE_NGA_ | mCsmGsmCsUCCAAGAGCCACCUUCCGUUUUAGAGCUAGA | 1049 |
| 20nt_4-9_006- | AAUAGCAAGUUAAAAUAAGGCUAGUCCGUUAUCAACUUGA | |
| 31947477_spCas9 | AAAAGUGGCACCGAGUCGGUGCUsmUsmUsmU | |
| GuideDesign10_CBE_NGA_ | mGsmCsmGsGUUCCAGCCUUCAGGAGGUUUUAGAGCUAGA | 1050 |
| 20nt_4-9_006- | AAUAGCAAGUUAAAAUAAGGCUAGUCCGUUAUCAACUUGA | |
| 31948907_spCas9 | AAAAGUGGCACCGAGUCGGUGCUsmUsmUsmU | |
| GuideDesign10 CBE_NGA_ | mGsmUsmUsCCCAAACCAUGCCACAGGUUUUAGAGCUAGA | 1051 |
| 20nt_4-9_006- | AAUAGCAAGUUAAAAUAAGGCUAGUCCGUUAUCAACUUGA | |
| 31950068_spCas9 | AAAAGUGGCACCGAGUCGGUGCUsmUsmUsmU | |
| GuideDesign10_CBE_NGA_ | mGsmUsmGsUUCCCAAACCAUGCCACGUUUUAGAGCUAGA | 1052 |
| 20nt_4-9_006- | AAUAGCAAGUUAAAAUAAGGCUAGUCCGUUAUCAACUUGA | |
| 31950070_spCas9 | AAAAGUGGCACCGAGUCGGUGCUsmUsmUsmU | |
| GuideDesign10 CBE_NGA_ | mCsmAsmGsCCAGGGCAGCACUUGAAGUUUUAGAGCUAGA | 1053 |
| 20nt_4-9_006- | AAUAGCAAGUUAAAAUAAGGCUAGUCCGUUAUCAACUUGA | |
| 31951968_spCas9 | AAAAGUGGCACCGAGUCGGUGCUsmUsmUsmU | |
| GuideDesign10_CBE_NGA_ | mCsmCsmUsUCAGCCAGGGCAGCACUGUUUUAGAGCUAGA | 1054 |
| 20nt_4-9_006- | AAUAGCAAGUUAAAAUAAGGCUAGUCCGUUAUCAACUUGA | |
| 31951972_spCas9 | AAAAGUGGCACCGAGUCGGUGCUsmUsmUsmU | |
| GuideDesign10_CBE_NGA_ | mCsmCsmCsCAGGGAUCCUGCUCUCUGUUUUAGAGCUAGA | 1055 |
| 20nt_4- | AAUAGCAAGUUAAAAUAAGGCUAGUCCGUUAUCAACUUGA | |
| 9_006_+_31946404_spCas9 | AAAAGUGGCACCGAGUCGGUGCUsmUsmUsmU | |
| GuideDesign10_CBE_NGA_ | mUsmCsmAsAGACCAAAAGACUGUCAGUUUUAGAGCUAGA | 1056 |
| 20nt_4- | AAUAGCAAGUUAAAAUAAGGCUAGUCCGUUAUCAACUUGA | |
| 9_006_+_31946568_spCas9 | AAAAGUGGCACCGAGUCGGUGCUsmUsmUsmU | |
| GuideDesign10_CBE_NGA_ | mGsmCsmAsCCUGCCAAGUGAAUGGCGUUUUAGAGCUAGA | 1057 |
| 20nt_4- | AAUAGCAAGUUAAAAUAAGGCUAGUCCGUUAUCAACUUGA | |
| 9_006_+_31947135_spCas9 | AAAAGUGGCACCGAGUCGGUGCUsmUsmUsmU | |
| GuideDesign10_CBE_NGA_ | mCsmUsmGsCCAAGUGAAUGGCCGAUGUUUUAGAGCUAGA | 1058 |
| 20nt_4- | AAUAGCAAGUUAAAAUAAGGCUAGUCCGUUAUCAACUUGA | |
| 9_006_+_31947139_spCas9 | AAAAGUGGCACCGAGUCGGUGCUsmUsmUsmU | |
| GuideDesign10_CBE_NGA_ | mGsmAsmAsUGGCCGAUGGAGUGGGCGUUUUAGAGCUAGA | 1059 |
| 20nt_4- | AAUAGCAAGUUAAAAUAAGGCUAGUCCGUUAUCAACUUGA | |
| 9_006_+_31947148_spCas9 | AAAAGUGGCACCGAGUCGGUGCUsmUsmUsmU | |
| GuideDesign10_CBE_NGA_ | mAsmGsmUsGGGCAGACAGCGAUCUGGUUUUAGAGCUAGA | 1060 |
| 20nt_4- | AAUAGCAAGUUAAAAUAAGGCUAGUCCGUUAUCAACUUGA | |
| 9_006_+_31947161_spCas9 | AAAAGUGGCACCGAGUCGGUGCUsmUsmUsmU | |
| GuideDesign10_CBE_NGA_ | mGsmGsmCsAGCCAGUACCGCCUUGAGUUUUAGAGCUAGA | 1061 |
| 20nt_4- | AAUAGCAAGUUAAAAUAAGGCUAGUCCGUUAUCAACUUGA | |
| 9_006_+_31947394_spCas9 | AAAAGUGGCACCGAGUCGGUGCUsmUsmUsmU | |
| GuideDesign10_CBE_NGA_ | mUsmCsmCsCAGCGGCGAACGUGUCAGUUUUAGAGCUAGA | 1062 |
| 20nt_4- | AAUAGCAAGUUAAAAUAAGGCUAGUCCGUUAUCAACUUGA | |
| 9_006_+_31947460_spCas9 | AAAAGUGGCACCGAGUCGGUGCUsmUsmUsmU | |
| GuideDesign10_CBE_NGA_ | mGsmUsmGsUCAGGAAGGUGGCUCUUGUUUUAGAGCUAGA | 1063 |
| 20nt_4- | AAUAGCAAGUUAAAAUAAGGCUAGUCCGUUAUCAACUUGA | |
| 9_006_+_31947474_spCas9 | AAAAGUGGCACCGAGUCGGUGCUsmUsmUsmU | |
| GuideDesign10_CBE_NGA_ | mAsmGsmGsGGAACAACAGAAGCGGAGUUUUAGAGCUAGA | 1064 |
| 20nt_4- | AAUAGCAAGUUAAAAUAAGGCUAGUCCGUUAUCAACUUGA | |
| 9_006_+_31947942_spCas9 | AAAAGUGGCACCGAGUCGGUGCUsmUsmUsmU | |
| GuideDesign10_CBE_NGA_ | mCsmCsmUsCCAGGCAGUGUACAGCAGUUUUAGAGCUAGA | 1065 |
| 20nt_4- | AAUAGCAAGUUAAAAUAAGGCUAGUCCGUUAUCAACUUGA | |
| 9_006_+_31948866_spCas9 | AAAAGUGGCACCGAGUCGGUGCUsmUsmUsmU | |
| GuideDesign10_CBE_NGA_ | mUsmUsmCSUACCAAAUGAUCGGUAGGUUUUAGAGCUAGA | 1066 |
| 20nt_4- | AAUAGCAAGUUAAAAUAAGGCUAGUCCGUUAUCAACUUGA | |
| 9_006_+_31949541_spCas9 | AAAAGUGGCACCGAGUCGGUGCUsmUsmUsmU | |
| GuideDesign10_CBE_NGA_ | mCsmUsmAsCCAAAUGAUCGGUAGGGGUUUUAGAGCUAGA | 1067 |
| 20nt_4- | AAUAGCAAGUUAAAAUAAGGCUAGUCCGUUAUCAACUUGA | |
| 9_006_+_31949543_spCas9 | AAAAGUGGCACCGAGUCGGUGCUsmUsmUsmU | |
| GuideDesign10_CBE_NGA_ | mCsmAsmAsGCAACCAUGGCAGGCCAGUUUUAGAGCUAGA | 1068 |
| 20nt_4- | AAUAGCAAGUUAAAAUAAGGCUAGUCCGUUAUCAACUUGA | |
| 9_006_+_31950113_spCas9 | AAAAGUGGCACCGAGUCGGUGCUsmUsmUsmU | |
| GuideDesign10_CBE_NGA_ | mUsmAsmUsGGCCAGACUAUCAGGUGGUUUUAGAGCUAGA | 1069 |
| 20nt_4- | AAUAGCAAGUUAAAAUAAGGCUAGUCCGUUAUCAACUUGA | |
| 9_006_+_31950755_spCas9 | AAAAGUGGCACCGAGUCGGUGCUsmUsmUsmU | |
| GuideDesign10_CBE_NGA_ | mCsmAsmCsUUGCCAGCAACAAAGUAGUUUUAGAGCUAGA | 1070 |
| 20nt_4- | AAUAGCAAGUUAAAAUAAGGCUAGUCCGUUAUCAACUUGA | |
| 9_006_+_31950927_spCas9 | AAAAGUGGCACCGAGUCGGUGCUsmUsmUsmU | |
| GuideDesign10_CBE_NGA_ | mGsmCsmUsCAAUAUGCCCCAGGCUAGUUUUAGAGCUAGA | 1071 |
| 20nt_4- | AAUAGCAAGUUAAAAUAAGGCUAGUCCGUUAUCAACUUGA | |
| 9_006_+_31951361_spCas9 | AAAAGUGGCACCGAGUCGGUGCUsmUsmUsmU | |
| GuideDesign10_CBE_NGA_ | mCsmUsmUsUCAAGUGCUGCCCUGGCGUUUUAGAGCUAGA | 1072 |
| 20nt_4- | AAUAGCAAGUUAAAAUAAGGCUAGUCCGUUAUCAACUUGA | |
| 9_006_+_31951969_spCas9 | AAAAGUGGCACCGAGUCGGUGCUsmUsmUsmU | |
| GuideDesign10_CBE_NGG_ | mCsmAsmCsUCCAUCGGCCAUUCACUGUUUUAGAGCUAGA | 1073 |
| 20nt_4-9_003- | AAUAGCAAGUUAAAAUAAGGCUAGUCCGUUAUCAACUUGA | |
| 31947142_spCas9 | AAAAGUGGCACCGAGUCGGUGCUsmUsmUsmU | |
| GuideDesign10_CBE_NGG_ | mGsmAsmCsCCAAAUUUUGGGGUAUGGUUUUAGAGCUAGA | 1074 |
| 20nt_4-9_003 | AAUAGCAAGUUAAAAUAAGGCUAGUCCGUUAUCAACUUGA | |
| 31948419_spCas9 | AAAAGUGGCACCGAGUCGGUGCUsmUsmUsmU | |
| GuideDesign10_CBE_NGG_ | mUsmGsmCsGGUUCCAGCCUUCAGGAGUUUUAGAGCUAGA | 1075 |
| 20nt_4-9_003- | AAUAGCAAGUUAAAAUAAGGCUAGUCCGUUAUCAACUUGA | |
| 31948908_spCas9 | AAAAGUGGCACCGAGUCGGUGCUsmUsmUsmU | |
| GuideDesign10_CBE_NGG_ | mGsmUsmGsCGGUUCCAGCCUUCAGGGUUUUAGAGCUAGA | 1076 |
| 20nt_4-9_003- | AAUAGCAAGUUAAAAUAAGGCUAGUCCGUUAUCAACUUGA | |
| 31948909_spCas9 | AAAAGUGGCACCGAGUCGGUGCUsmUsmUsmU | |
| GuideDesign10_CBE_NGG_ | mUsmGsmGsCCUGCCAUGGUUGCUUGGUUUUAGAGCUAGA | 1077 |
| 20nt_4-9_003- | AAUAGCAAGUUAAAAUAAGGCUAGUCCGUUAUCAACUUGA | |
| 31950110_spCas9 | AAAAGUGGCACCGAGUCGGUGCUsmUsmUsmU | |
| GuideDesign10_CBE_NGG_ | mCsmUsmCsCUUCCGACUUCUCCAAGGUUUUAGAGCUAGA | 1078 |
| 20nt_4- | AAUAGCAAGUUAAAAUAAGGCUAGUCCGUUAUCAACUUGA | |
| 9_003_+_31946448_spCas9 | AAAAGUGGCACCGAGUCGGUGCUsmUsmUsmU | |
| GuideDesign10_CBE_NGG_ | mUsmCsmCsUUCCGACUUCUCCAAGAGUUUUAGAGCUAGA | 1079 |
| 20nt_4- | AAUAGCAAGUUAAAAUAAGGCUAGUCCGUUAUCAACUUGA | |
| 9_003_+_31946449_spCas9 | AAAAGUGGCACCGAGUCGGUGCUsmUsmUsmU | |
| GuideDesign10_CBE_NGG_ | mUsmCsmUsCCAAGAGGGCCAGGCACGUUUUAGAGCUAGA | 1080 |
| 20nt_4- | AAUAGCAAGUUAAAAUAAGGCUAGUCCGUUAUCAACUUGA | |
| 9_003_+_31946460_spCas9 | AAAAGUGGCACCGAGUCGGUGCUsmUsmUsmU | |
| GuideDesign10_CBE_NGG_ | mCsmUsmCsAAGACCAAAAGACUGUCGUUUUAGAGCUAGA | 1081 |
| 20nt_4- | AAUAGCAAGUUAAAAUAAGGCUAGUCCGUUAUCAACUUGA | |
| 9_003_+_31946567_spCas9 | AAAAGUGGCACCGAGUCGGUGCUsmUsmUsmU | |
| GuideDesign10_CBE_NGG_ | mAsmGsmAsCCAAAAGACUGUCAGGAGUUUUAGAGCUAGA | 1082 |
| 20nt_4- | AAUAGCAAGUUAAAAUAAGGCUAGUCCGUUAUCAACUUGA | |
| 9_003_+_31946571_spCas9 | AAAAGUGGCACCGAGUCGGUGCUsmUsmUsmU | |
| GuideDesign10_CBE_NGG_ | mCsmCsmUsGCCAAGUGAAUGGCCGAGUUUUAGAGCUAGA | 1083 |
| 20nt_4- | AAUAGCAAGUUAAAAUAAGGCUAGUCCGUUAUCAACUUGA | |
| 9_003_+_31947138_spCas9 | AAAAGUGGCACCGAGUCGGUGCUsmUsmUsmU | |
| GuideDesign10_CBE_NGG_ | mCsmUsmCsCCAGCGGCGAACGUGUCGUUUUAGAGCUAGA | 1084 |
| 20nt_4- | AAUAGCAAGUUAAAAUAAGGCUAGUCCGUUAUCAACUUGA | |
| 9_003_+_31947459_spCas9 | AAAAGUGGCACCGAGUCGGUGCUsmUsmUsmU | |
| GuideDesign10_CBE_NGG_ | mCsmAsmGsCGGCGAACGUGUCAGGAGUUUUAGAGCUAGA | 1085 |
| 20nt_4- | AAUAGCAAGUUAAAAUAAGGCUAGUCCGUUAUCAACUUGA | |
| 9_003_+_31947463_spCas9 | AAAAGUGGCACCGAGUCGGUGCUsmUsmUsmU | |
| GuideDesign10_CBE_NGG_ | mCsmGsmGsCGAACGUGUCAGGAAGGGUUUUAGAGCUAGA | 1086 |
| 20nt_4- | AAUAGCAAGUUAAAAUAAGGCUAGUCCGUUAUCAACUUGA | |
| 9_003_+_31947466_spCas9 | AAAAGUGGCACCGAGUCGGUGCUsmUsmUsmU | |
| GuideDesign10_CBE_NGG_ | mCsmGsmUsGUCAGGAAGGUGGCUCUGUUUUAGAGCUAGA | 1087 |
| 20nt_4- | AAUAGCAAGUUAAAAUAAGGCUAGUCCGUUAUCAACUUGA | |
| 9_003_+_31947473_spCas9 | AAAAGUGGCACCGAGUCGGUGCUsmUsmUsmU | |
| GuideDesign10_CBE_NGG_ | mUsmUsmUsUCUACCAAAUGAUCGGUGUUUUAGAGCUAGA | 1088 |
| 20nt_4- | AAUAGCAAGUUAAAAUAAGGCUAGUCCGUUAUCAACUUGA | |
| 9_003_+_31949539_spCas9 | AAAAGUGGCACCGAGUCGGUGCUsmUsmUsmU | |
| GuideDesign10_CBE_NGG_ | mUsmUsmUsCUACCAAAUGAUCGGUAGUUUUAGAGCUAGA | 1089 |
| 20nt_4- | AAUAGCAAGUUAAAAUAAGGCUAGUCCGUUAUCAACUUGA | |
| 9_003_+_31949540_spCas9 | AAAAGUGGCACCGAGUCGGUGCUsmUsmUsmU | |
| GuideDesign10_CBE_NGG_ | mGsmCsmAsACAAAGUAAGACAUACUGUUUUAGAGCUAGA | 1090 |
| 20nt_4- | AAUAGCAAGUUAAAAUAAGGCUAGUCCGUUAUCAACUUGA | |
| 9_003_+_31950936_spCas9 | AAAAGUGGCACCGAGUCGGUGCUsmUsmUsmU | |
| GuideDesign10_CBE_NGG_ | mAsmCsmCsUCUUUCAAGUGCUGCCCGUUUUAGAGCUAGA | 1091 |
| 20nt_4- | AAUAGCAAGUUAAAAUAAGGCUAGUCCGUUAUCAACUUGA | |
| 9_003_+_31951965_spCas9 | AAAAGUGGCACCGAGUCGGUGCUsmUsmUsmU | |
| GuideDesign10_CBE_NGG_ | mGsmAsmAsACUCCAAGAUGAGGAUUGUUUUAGAGCUAGA | 1092 |
| 20nt_4- | AAUAGCAAGUUAAAAUAAGGCUAGUCCGUUAUCAACUUGA | |
| 9_003_+_31951996_spCas9 | AAAAGUGGCACCGAGUCGGUGCUsmUsmUsmU | |
| GuideDesign10_CBE_NGG_ | mAsmAsmAsCUCCAAGAUGAGGAUUUGUUUUAGAGCUAGA | 1093 |
| 20nt_4- | AAUAGCAAGUUAAAAUAAGGCUAGUCCGUUAUCAACUUGA | |
| 9_003_+_31951997_spCas9 | AAAAGUGGCACCGAGUCGGUGCUsmUsmUsmU | |
| GuideDesign3_CBE_NNGRR | mUsmUsmCsUCCAAGAGGGCCAGGCACGUUUUAGUACUCU | 1094 |
| 21nt_3- | GUAAUGAAAAUUACAGAAUCUACUAAAACAAGGCAAAAUG | |
| 12_012_+_31946459_saCas9 | CCGUGUUUAUCUCGUCAACUUGUUGGCGAGAUsmUsmUsm | |
| | U | |
| GuideDesign3_CBE_NNGRR | mUsmUsmCsUCCAAGAGGGCCAGGCACGUUUUAGUACUCU | 1095 |
| 21nt_3- | GUAAUGAAAAUUACAGAAUCUACUAAAACAAGGCAAAAUG | |
| 12_012_+_31946459_saCas9 | CCGUGUUUAUCUCGUCAACUUGUUGGCGAGAUsmUsmUsm | |
| | U | |
| GuideDesign3_CBE_NNGRR | mAsmCsmCsAAAAGACUGUCAGGAAGGGUUUUAGUACUCU | 1096 |
| 21nt_3- | GUAAUGAAAAUUACAGAAUCUACUAAAACAAGGCAAAAUG | |
| 12_012_+_31946573_saCas9 | CCGUGUUUAUCUCGUCAACUUGUUGGCGAGAUsmUsmUsm | |
| | U | |
| GuideDesign3_CBE_NNGRR | mAsmCsmCsAAAAGACUGUCAGGAAGGGUUUUAGUACUCU | 1097 |
| 21nt_3- | GUAAUGAAAAUUACAGAAUCUACUAAAACAAGGCAAAAUG | |
| 12_012_+_31946573_saCas9 | CCGUGUUUAUCUCGUCAACUUGUUGGCGAGAUsmUsmUsm | |
| | U | |
| GuideDesign3_CBE_NNGRR | mAsmCsmCsUGCCAAGUGAAUGGCCGAGUUUUAGUACUCU | 1098 |
| 21nt_3- | GUAAUGAAAAUUACAGAAUCUACUAAAACAAGGCAAAAUG | |
| 12_012_+_31947137_saCas9 | CCGUGUUUAUCUCGUCAACUUGUUGGCGAGAUsmUsmUsm | |
| | U | |
| GuideDesign3_CBE_NNGRR | mAsmCsmCsUGCCAAGUGAAUGGCCGAGUUUUAGUACUCU | 1099 |
| 21nt_3- | GUAAUGAAAAUUACAGAAUCUACUAAAACAAGGCAAAAUG | |
| 12_012_+_31947137_saCas9 | CCGUGUUUAUCUCGUCAACUUGUUGGCGAGAUsmUsmUsm | |
| | U | |
| GuideDesign3_CBE_NNGRR | mGsmAsmGsCAACAUGUGUUCAAAGUCGUUUUAGUACUCU | 1100 |
| 21nt_3- | GUAAUGAAAAUUACAGAAUCUACUAAAACAAGGCAAAAUG | |
| 12_012_+_31949493_saCas9 | CCGUGUUUAUCUCGUCAACUUGUUGGCGAGAUsmUsmUsm | |
| | U | |
| GuideDesign3_CBE_NNGRR | mGsmAsmGsCAACAUGUGUUCAAAGUCGUUUUAGUACUCU | 1101 |
| 21nt_3- | GUAAUGAAAAUUACAGAAUCUACUAAAACAAGGCAAAAUG | |
| 12_012_+_31949493_saCas9 | CCGUGUUUAUCUCGUCAACUUGUUGGCGAGAUsmUsmUsm | |
| | U | |
| GuideDesign3_CBE_NNNRRT_ | mGsmCsmUsCCAGGACCCCGUAGAUCUGUUUUAGUACUCU | 1102 |
| 21nt_3-12_015- | GUAAUGAAAAUUACAGAAUCUACUAAAACAAGGCAAAAUG | |
| 31946530_saCas9 | CCGUGUUUAUCUCGUCAACUUGUUGGCGAGAUsmUsmUsm | |
| | U | |
| GuideDesign3_CBE_NNNRRT_ | mGsmCsmUsCCAGGACCCCGUAGAUCUGUUUUAGUACUCU | 1103 |
| 21nt_3-12_015- | GUAAUGAAAAUUACAGAAUCUACUAAAACAAGGCAAAAUG | |
| 31946530_saCas9 | CCGUGUUUAUCUCGUCAACUUGUUGGCGAGAUsmUsmUsm | |
| | U | |
| GuideDesign3_CBE_NNNRRT_ | mUsmUsmCsAGGGUGCUCCAGGACCCCGUUUUAGUACUCU | 1104 |
| 21nt_3-12_015- | GUAAUGAAAAUUACAGAAUCUACUAAAACAAGGCAAAAUG | |
| 31946538_saCas9 | CCGUGUUUAUCUCGUCAACUUGUUGGCGAGAUsmUsmUsm | |
| | U | |
| GuideDesign3_CBE_NNNRRT_ | mUsmUsmCsAGGGUGCUCCAGGACCCCGUUUUAGUACUCU | 1105 |
| 21nt_3-12_015- | GUAAUGAAAAUUACAGAAUCUACUAAAACAAGGCAAAAUG | |
| 31946538_saCas9 | CCGUGUUUAUCUCGUCAACUUGUUGGCGAGAUsmUsmUsm | |
| | U | |
| GuideDesign3_CBE_NNNRRT_ | mGsmGsmGsGCCAGUAUUCCCCGUUCUGUUUUAGUACUCU | 1106 |
| 21nt_3-12_015_- | GUAAUGAAAAUUACAGAAUCUACUAAAACAAGGCAAAAUG | |
| 31947030_saCas9 | CCGUGUUUAUCUCGUCAACUUGUUGGCGAGAUsmUsmUsm | |
| | U | |
| GuideDesign3_CBE_NNNRRT_ | mGsmGsmGsGCCAGUAUUCCCCGUUCUGUUUUAGUACUCU | 1107 |
| 21nt_3-12_015- | GUAAUGAAAAUUACAGAAUCUACUAAAACAAGGCAAAAUG | |
| 31947030_saCas9 | CCGUGUUUAUCUCGUCAACUUGUUGGCGAGAUsmUsmUsm | |
| | U | |
| GuideDesign3_CBE_NNNRRT_ | mAsmCsmUsCCAUCGGCCAUUCACUUGGUUUUAGUACUCU | 1108 |
| 21nt_3-12_015_- | GUAAUGAAAAUUACAGAAUCUACUAAAACAAGGCAAAAUG | |
| 31947137_saCas9 | CCGUGUUUAUCUCGUCAACUUGUUGGCGAGAUsmUsmUsm | |
| | U | |
| GuideDesign3_CBE_NNNRRT_ | mAsmCsmUsCCAUCGGCCAUUCACUUGGUUUUAGUACUCU | 1109 |
| 21nt_3-12_015- | GUAAUGAAAAUUACAGAAUCUACUAAAACAAGGCAAAAUG | |
| 31947137_saCas9 | CCGUGUUUAUCUCGUCAACUUGUUGGCGAGAUsmUsmUsm | |
| | U | |
| GuideDesign3_CBE_NNNRRT_ | mUsmGsmGsCCUGCCAUGGUUGCUUGUGUUUUAGUACUCU | 1110 |
| 21nt_3-12_015- | GUAAUGAAAAUUACAGAAUCUACUAAAACAAGGCAAAAUG | |
| 31950106_saCas9 | CCGUGUUUAUCUCGUCAACUUGUUGGCGAGAUsmUsmUsm | |
| | U | |
| GuideDesign3_CBE_NNNRRT_ | mUsmGsmGsCCUGCCAUGGUUGCUUGUGUUUUAGUACUCU | 1111 |
| 21nt_3-12_015- | GUAAUGAAAAUUACAGAAUCUACUAAAACAAGGCAAAAUG | |
| 31950106_saCas9 | CCGUGUUUAUCUCGUCAACUUGUUGGCGAGAUsmUsmUsm | |
| | U | |
| GuideDesign3_CBE_NNNRRT_ | mUsmCsmUsUGGCCUGCCAUGGUUGCUGUUUUAGUACUCU | 1112 |
| 21nt_3-12_015- | GUAAUGAAAAUUACAGAAUCUACUAAAACAAGGCAAAAUG | |
| 31950109_saCas9 | CCGUGUUUAUCUCGUCAACUUGUUGGCGAGAUsmUsmUsm | |
| | U | |
| GuideDesign3_CBE_NNNRRT_ | mUsmCsmUsUGGCCUGCCAUGGUUGCUGUUUUAGUACUCU | 1113 |
| 21nt_3-12_015- | GUAAUGAAAAUUACAGAAUCUACUAAAACAAGGCAAAAUG | |
| 31950109_saCas9 | CCGUGUUUAUCUCGUCAACUUGUUGGCGAGAUsmUsmUsm | |
| | U | |
| GuideDesign3_CBE_NNNRRT_ | mUsmCsmAsGCCAGGGCAGCACUUGAAGUUUUAGUACUCU | 1114 |
| 21nt_3-12_015- | GUAAUGAAAAUUACAGAAUCUACUAAAACAAGGCAAAAUG | |
| 31951965_saCas9 | CCGUGUUUAUCUCGUCAACUUGUUGGCGAGAUsmUsmUsm | |
| | U | |
| GuideDesign3_CBE_NNNRRT_ | mUsmCsmAsGCCAGGGCAGCACUUGAAGUUUUAGUACUCU | 1115 |
| 21nt_3-12_015- | GUAAUGAAAAUUACAGAAUCUACUAAAACAAGGCAAAAUG | |
| 31951965_saCas9 | CCGUGUUUAUCUCGUCAACUUGUUGGCGAGAUsmUsmUsm | |
| | U | |
| GuideDesign3_CBE_NNNRRT_ | mAsmUsmCsGCACCUGCCAAGUGAAUGGUUUUAGUACUCU | 1116 |
| 21nt_3- | GUAAUGAAAAUUACAGAAUCUACUAAAACAAGGCAAAAUG | |
| 12_015_+_31947132_saCas9 | CCGUGUUUAUCUCGUCAACUUGUUGGCGAGAUsmUsmUsm | |
| | U | |
| GuideDesign3_CBE_NNNRRT_ | mAsmUsmCsGCACCUGCCAAGUGAAUGGUUUUAGUACUCU | 1117 |
| 21nt_3- | GUAAUGAAAAUUACAGAAUCUACUAAAACAAGGCAAAAUG | |
| 12_015_+_31947132_saCas9 | CCGUGUUUAUCUCGUCAACUUGUUGGCGAGAUsmUsmUsm | |
| | U | |
| GuideDesign3_CBE_NNNRRT_ | mUsmGsmGsCCGAUGGAGUGGGCAGACGUUUUAGUACUCU | 1118 |
| 21nt_3- | GUAAUGAAAAUUACAGAAUCUACUAAAACAAGGCAAAAUG | |
| 12_015_+_31947151_saCas9 | CCGUGUUUAUCUCGUCAACUUGUUGGCGAGAUsmUsmUsm | |
| | U | |
| GuideDesign3_CBE_NNNRRT_ | mUsmGsmGsCCGAUGGAGUGGGCAGACGUUUUAGUACUCU | 1119 |
| 21nt_3- | GUAAUGAAAAUUACAGAAUCUACUAAAACAAGGCAAAAUG | |
| 12_015_+_31947151_saCas9 | CCGUGUUUAUCUCGUCAACUUGUUGGCGAGAUsmUsmUsm | |
| | U | |
| GuideDesign3_CBE_NNNRRT_ | mUsmCsmCsCAGCGGCGAACGUGUCAGGUUUUAGUACUCU | 1120 |
| 21nt_3- | GUAAUGAAAAUUACAGAAUCUACUAAAACAAGGCAAAAUG | |
| 12_015_+_31947460_saCas9 | CCGUGUUUAUCUCGUCAACUUGUUGGCGAGAUsmUsmUsm | |
| | U | |
| GuideDesign3_CBE_NNNRRT_ | mUsmCsmCsCAGCGGCGAACGUGUCAGGUUUUAGUACUCU | 1121 |
| 21nt_3- | GUAAUGAAAAUUACAGAAUCUACUAAAACAAGGCAAAAUG | |
| 12_015_+_31947460_saCas9 | CCGUGUUUAUCUCGUCAACUUGUUGGCGAGAUsmUsmUsm | |
| | U | |
| GuideDesign3_CBE_NNNRRT_ | mCsmCsmCsAGGGGAACAACAGAAGCGGUUUUAGUACUCU | 1122 |
| 21nt_3- | GUAAUGAAAAUUACAGAAUCUACUAAAACAAGGCAAAAUG | |
| 12_015_+_31947939_saCas9 | CCGUGUUUAUCUCGUCAACUUGUUGGCGAGAUsmUsmUsm | |
| | U | |
| GuideDesign3_CBE_NNNRRT_ | mCsmCsmCsAGGGGAACAACAGAAGCGGUUUUAGUACUCU | 1123 |
| 21nt_3- | GUAAUGAAAAUUACAGAAUCUACUAAAACAAGGCAAAAUG | |
| 12_015_+_31947939_saCas9 | CCGUGUUUAUCUCGUCAACUUGUUGGCGAGAUsmUsmUsm | |
| | U | |
| GuideDesign3_CBE_NNNRRT_ | mGsmUsmCsACGAAGCAGCUCAAUGAAGUUUUAGUACUCU | 1124 |
| 21nt_3- | GUAAUGAAAAUUACAGAAUCUACUAAAACAAGGCAAAAUG | |
| 12_015_+_31948478_saCas9 | CCGUGUUUAUCUCGUCAACUUGUUGGCGAGAUsmUsmUsm | |
| | U | |
| GuideDesign3_CBE_NNNRRT_ | mGsmUsmCsACGAAGCAGCUCAAUGAAGUUUUAGUACUCU | 1125 |
| 21nt_3- | GUAAUGAAAAUUACAGAAUCUACUAAAACAAGGCAAAAUG | |
| 12_015_+_31948478_saCas9 | CCGUGUUUAUCUCGUCAACUUGUUGGCGAGAUsmUsmUsm | |
| | U | |
| GuideDesign3_CBE_NNNRRT_ | mGsmGsmCsCCUCCAGGCAGUGUACAGGUUUUAGUACUCU | 1126 |
| 21nt_3- | GUAAUGAAAAUUACAGAAUCUACUAAAACAAGGCAAAAUG | |
| 12_015_+_31948863_saCas9 | CCGUGUUUAUCUCGUCAACUUGUUGGCGAGAUsmUsmUsm | |
| | U | |
| GuideDesign3_CBE_NNNRRT_ | mGsmGsmCsCCUCCAGGCAGUGUACAGGUUUUAGUACUCU | 1127 |
| 21nt_3- | GUAAUGAAAAUUACAGAAUCUACUAAAACAAGGCAAAAUG | |
| 12_015_+_31948863_saCas9 | CCGUGUUUAUCUCGUCAACUUGUUGGCGAGAUsmUsmUsm | |
| | U | |
| GuideDesign3_CBE_NNNRRT_ | mUsmUsmUsCUACCAAAUGAUCGGUAGGUUUUAGUACUCU | 1128 |
| 21nt_3- | GUAAUGAAAAUUACAGAAUCUACUAAAACAAGGCAAAAUG | |
| 12_015_+_31949540_saCas9 | CCGUGUUUAUCUCGUCAACUUGUUGGCGAGAUsmUsmUsm | |
| | U | |
| GuideDesign3_CBE_NNNRRT_ | mUsmUsmUsCUACCAAAUGAUCGGUAGGUUUUAGUACUCU | 1129 |
| 21nt_3- | GUAAUGAAAAUUACAGAAUCUACUAAAACAAGGCAAAAUG | |
| 12_015_+_31949540_saCas9 | CCGUGUUUAUCUCGUCAACUUGUUGGCGAGAUsmUsmUsm | |
| | U | |
| GuideDesign3_CBE_NNNRRT_ | mGsmCsmAsACCAUGGCAGGCCAAGAUGUUUUAGUACUCU | 1130 |
| 21nt_3- | GUAAUGAAAAUUACAGAAUCUACUAAAACAAGGCAAAAUG | |
| 12_015_+_31950116_saCas9 | CCGUGUUUAUCUCGUCAACUUGUUGGCGAGAUsmUsmUsm | |
| | U | |
| GuideDesign3_CBE_NNNRRT_ | mGsmCsmAsACCAUGGCAGGCCAAGAUGUUUUAGUACUCU | 1131 |
| 21nt_3- | GUAAUGAAAAUUACAGAAUCUACUAAAACAAGGCAAAAUG | |
| 12_015_+_31950116_saCas9 | CCGUGUUUAUCUCGUCAACUUGUUGGCGAGAUsmUsmUsm | |
| | U | |

⁵“mN” indicates a 2′-OMe modification of the nucleotide “N”, and “Ns” indicates that
the nucleotide “N” is linked to the following (i.e., 3′) nucleotide by a phosphorothioate
(PS).

TABLE 1F
Representative sequences for guide polynucleotides for use in guiding a base
editor to alter a start codon or TATA box of a complement factor B polynucleotide
using base editing.⁶

| | | SEQ |
|---|---|---|
| | | ID |
| Guide polynucleotide name | Guide polynucleotide sequence | NO |
| GuideDesign90_ABE_NGA | mAsmAsmAsAGGCUGGGGGCUGGUGGGUUUUAGAGCUAGAA | 1132 |
| _20nt_3- | AUAGCAAGUUAAAAUAAGGCUAGUCCGUUAUCAACUUGAAA | |
| 12_019_+_31946067_spCas9 | AAGUGGCACCGAGUCGGUGCmUsmUsmUsU | |
| GuideDesign90_CBE_NGA | mAsmAsmAsAGGCUGGGGGCUGGUGGGUUUUAGAGCUAGAA | 1133 |
| _20nt_4- | AUAGCAAGUUAAAAUAAGGCUAGUCCGUUAUCAACUUGAAA | |
| 9_006_+_31946067_spCas9 | AAGUGGCACCGAGUCGGUGCmUsmUsmUSU | |
| GuideDesign90_CBE_NGA | mAsmAsmAsAGGCUGGGGGCUGGUGGGUUUUAGAGCUAGAA | 1134 |
| _20nt_3- | AUAGCAAGUUAAAAUAAGGCUAGUCCGUUAUCAACUUGAAA | |
| 16_035_+_31946067_spCas9 | AAGUGGCACCGAGUCGGUGCmUsmUsmUsU | |
| GuideDesign90_ABE_NG | mAsmAsmAsAGGCUGGGGGCUGGUGGGUUUUAGAGCUAGAA | 1135 |
| _20nt_3- | AUAGCAAGUUAAAAUAAGGCUAGUCCGUUAUCAACUUGAAA | |
| 9_027_+_31946067_spCas9 | AAGUGGCACCGAGUCGGUGCmUsmUsmUsU | |
| GuideDesign90_CBE_NG | mAsmAsmAsAGGCUGGGGGCUGGUGGGUUUUAGAGCUAGAA | 1136 |
| _20nt_4- | AUAGCAAGUUAAAAUAAGGCUAGUCCGUUAUCAACUUGAAA | |
| 9_028_+_31946067_spCas9 | AAGUGGCACCGAGUCGGUGCmUsmUsmUSU | |
| Guide_Design90_ABE_NRN | mAsmAsmAsAGGCUGGGGGCUGGUGGGUUUUAGAGCUAGAA | 1137 |
| _20nt_3- | AUAGCAAGUUAAAAUAAGGCUAGUCCGUUAUCAACUUGAAA | |
| 9_021_+_31946067_spCas9 | AAGUGGCACCGAGUCGGUGCmUsmUsmUsU | |
| GuideDesign90_CBE_NRN | mAsmAsmAsAGGCUGGGGGCUGGUGGGUUUUAGAGCUAGAA | 1138 |
| _20nt_3- | AUAGCAAGUUAAAAUAAGGCUAGUCCGUUAUCAACUUGAAA | |
| 9_022_+_31946067_spCas9 | AAGUGGCACCGAGUCGGUGCmUsmUsmUsU | |
| GuideDesign90_MBE_NRN | mAsmAsmAsAGGCUGGGGGCUGGUGGGUUUUAGAGCUAGAA | 1139 |
| _20nt_3- | AUAGCAAGUUAAAAUAAGGCUAGUCCGUUAUCAACUUGAAA | |
| 9_023_+_31946067_spCas9 | AAGUGGCACCGAGUCGGUGCmUsmUsmUsU | |
| GuideDesign90_ABE_NRN | mAsmAsmAsGGCUGGGGGCUGGUGGGGUUUUAGAGCUAGAA | 1140 |
| _20nt_3- | AUAGCAAGUUAAAAUAAGGCUAGUCCGUUAUCAACUUGAAA | |
| 9_021_+_31946068_spCas9 | AAGUGGCACCGAGUCGGUGCmUsmUsmUsU | |
| GuideDesign90_CBE_NRN | mAsmAsmAsGGCUGGGGGCUGGUGGGGUUUUAGAGCUAGAA | 1141 |
| _20nt_3- | AUAGCAAGUUAAAAUAAGGCUAGUCCGUUAUCAACUUGAAA | |
| 9_022_+_31946068_spCas9 | AAGUGGCACCGAGUCGGUGCmUsmUsmUsU | |
| GuideDesign90_MBE_NRN | mAsmAsmAsGGCUGGGGGCUGGUGGGGUUUUAGAGCUAGAA | 1142 |
| _20nt_3- | AUAGCAAGUUAAAAUAAGGCUAGUCCGUUAUCAACUUGAAA | |
| 9_023_+_31946068_spCas9 | AAGUGGCACCGAGUCGGUGCmUsmUsmUSU | |
| GuideDesign90_CBE_NRRH | mAsmAsmAsGGCUGGGGGCUGGUGGGGUUUUAGAGCUAGAA | 1143 |
| _20nt_3- | AUAGCAAGUUAAAAUAAGGCUAGUCCGUUAUCAACUUGAAA | |
| 9_031_+_31946068_spCas9 | AAGUGGCACCGAGUCGGUGCmUsmUsmUSU | |
| GuideDesign90_ABE_NGC | mAsmAsmGsGCUGGGGGCUGGUGGGGGUUUUAGAGCUAGAA | 1144 |
| _20nt_3- | AUAGCAAGUUAAAAUAAGGCUAGUCCGUUAUCAACUUGAAA | |
| 9_008_+_31946069_spCas9 | AAGUGGCACCGAGUCGGUGCmUsmUsmUsU | |
| GuideDesign90_ABE_NGC | mAsmAsmGsGCUGGGGGCUGGUGGGGGUUUUAGAGCUAGAA | 1145 |
| _20nt_3- | AUAGCAAGUUAAAAUAAGGCUAGUCCGUUAUCAACUUGAAA | |
| 12_020_+_31946069_spCas9 | AAGUGGCACCGAGUCGGUGCmUsmUsmUsU | |
| GuideDesign90_CBE_NGC | mAsmAsmGsGCUGGGGGCUGGUGGGGGUUUUAGAGCUAGAA | 1146 |
| _20nt_4- | AUAGCAAGUUAAAAUAAGGCUAGUCCGUUAUCAACUUGAAA | |
| 9_009_+_31946069_spCas9 | AAGUGGCACCGAGUCGGUGCmUsmUsmUsU | |
| GuideDesign90_ABE_NG | mAsmAsmGsGCUGGGGGCUGGUGGGGGUUUUAGAGCUAGAA | 1147 |
| _20nt_3- | AUAGCAAGUUAAAAUAAGGCUAGUCCGUUAUCAACUUGAAA | |
| 9_027_+_31946069_spCas9 | AAGUGGCACCGAGUCGGUGCmUsmUsmUsU | |
| GuideDesign90_CBE_NG | mAsmAsmGsGCUGGGGGCUGGUGGGGGUUUUAGAGCUAGAA | 1148 |
| _20nt_4- | AUAGCAAGUUAAAAUAAGGCUAGUCCGUUAUCAACUUGAAA | |
| 9_028_+_31946069_spCas9 | AAGUGGCACCGAGUCGGUGCmUsmUsmUsU | |
| GuideDesign90_ABE_NRN | mAsmAsmGsGCUGGGGGCUGGUGGGGGUUUUAGAGCUAGAA | 1149 |
| _20nt_3- | AUAGCAAGUUAAAAUAAGGCUAGUCCGUUAUCAACUUGAAA | |
| 9_021_+_31946069_spCas9 | AAGUGGCACCGAGUCGGUGCmUsmUsmUSU | |
| GuideDesign90_CBE_NRN | mAsmAsmGsGCUGGGGGCUGGUGGGGGUUUUAGAGCUAGAA | 1150 |
| _20nt_3- | AUAGCAAGUUAAAAUAAGGCUAGUCCGUUAUCAACUUGAAA | |
| 9_022_+_31946069_spCas9 | AAGUGGCACCGAGUCGGUGCmUsmUsmUsU | |
| GuideDesign90_MBE_NRN | mAsmAsmGsGCUGGGGGCUGGUGGGGGUUUUAGAGCUAGAA | 1151 |
| _20nt_3- | AUAGCAAGUUAAAAUAAGGCUAGUCCGUUAUCAACUUGAAA | |
| 9_023_+_31946069_spCas9 | AAGUGGCACCGAGUCGGUGCmUsmUsmUsU | |
| GuideDesign90_ABE_NRCH | mAsmAsmGsGCUGGGGGCUGGUGGGGGUUUUAGAGCUAGAA | 1152 |
| _20nt_3- | AUAGCAAGUUAAAAUAAGGCUAGUCCGUUAUCAACUUGAAA | |
| 9_024_+_31946069_spCas9 | AAGUGGCACCGAGUCGGUGCmUsmUsmUsU | |
| GuideDesign90_CBE_NRCH | mAsmAsmGsGCUGGGGGCUGGUGGGGGUUUUAGAGCUAGAA | 1153 |
| _20nt_3- | AUAGCAAGUUAAAAUAAGGCUAGUCCGUUAUCAACUUGAAA | |
| 9_030_+_31946069_spCas9 | AAGUGGCACCGAGUCGGUGCmUsmUsmUsU | |
| GuideDesign90_ABE_NGG | mAsmAsmUsCUGUGUUCUGGCACCUGGUUUUAGAGCUAGAA | 1154 |
| _20nt_3-12_018_−_ | AUAGCAAGUUAAAAUAAGGCUAGUCCGUUAUCAACUUGAAA | |
| 31946040_spCas9 | AAGUGGCACCGAGUCGGUGCmUsmUsmUsU | |
| GuideDesign90_CBE_NGG | mAsmAsmUsCUGUGUUCUGGCACCUGGUUUUAGAGCUAGAA | 1155 |
| _20nt_4-9_003_−_ | AUAGCAAGUUAAAAUAAGGCUAGUCCGUUAUCAACUUGAAA | |
| 31946040_spCas9 | AAGUGGCACCGAGUCGGUGCmUsmUsmUsU | |
| GuideDesign90_CBE_NGG | mAsmAsmUsCUGUGUUCUGGCACCUGGUUUUAGAGCUAGAA | 1156 |
| _20nt_3-9_029_−_ | AUAGCAAGUUAAAAUAAGGCUAGUCCGUUAUCAACUUGAAA | |
| 31946040_spCas9 | AAGUGGCACCGAGUCGGUGCmUsmUsmUsU | |
| GuideDesign90_CBE_NGG | mAsmAsmUsCUGUGUUCUGGCACCUGGUUUUAGAGCUAGAA | 1157 |
| _20nt_3-16_033_−_ | AUAGCAAGUUAAAAUAAGGCUAGUCCGUUAUCAACUUGAAA | |
| 31946040_SpCas9 | AAGUGGCACCGAGUCGGUGCmUsmUsmUsU | |
| GuideDesign90_ABE_NG | mAsmAsmUsCUGUGUUCUGGCACCUGGUUUUAGAGCUAGAA | 1158 |
| _20nt_3-9_027_−_ | AUAGCAAGUUAAAAUAAGGCUAGUCCGUUAUCAACUUGAAA | |
| 31946041_spCas9 | AAGUGGCACCGAGUCGGUGCmUsmUsmUsU | |
| GuideDesign90_CBE_NG | mAsmAsmUsCUGUGUUCUGGCACCUGGUUUUAGAGCUAGAA | 1159 |
| _20nt_4-9_028_−_ | AUAGCAAGUUAAAAUAAGGCUAGUCCGUUAUCAACUUGAAA | |
| 31946041_spCas9 | AAGUGGCACCGAGUCGGUGCmUsmUsmUsU | |
| GuideDesign90_ABE_NRN | mAsmAsmUsCUGUGUUCUGGCACCUGGUUUUAGAGCUAGAA | 1160 |
| _20nt_3-9_021_−_ | AUAGCAAGUUAAAAUAAGGCUAGUCCGUUAUCAACUUGAAA | |
| 31946040_spCas9 | AAGUGGCACCGAGUCGGUGCmUsmUsmUsU | |
| GuideDesign90_CBE_NRN | mAsmAsmUsCUGUGUUCUGGCACCUGGUUUUAGAGCUAGAA | 1161 |
| _20nt_3-9_022_−_ | AUAGCAAGUUAAAAUAAGGCUAGUCCGUUAUCAACUUGAAA | |
| 31946040_spCas9 | AAGUGGCACCGAGUCGGUGCmUsmUsmUSU | |
| Guide_Design90_MBE_NRN | mAsmAsmUsCUGUGUUCUGGCACCUGGUUUUAGAGCUAGAA | 1162 |
| _20nt_3-9_023_−_ | AUAGCAAGUUAAAAUAAGGCUAGUCCGUUAUCAACUUGAAA | |
| 31946040_spCas9 | AAGUGGCACCGAGUCGGUGCmUsmUsmUsU | |
| GuideDesign90_CBE_NRRH | mAsmAsmUsCUGUGUUCUGGCACCUGGUUUUAGAGCUAGAA | 1163 |
| _20nt_3-9_031_−_ | AUAGCAAGUUAAAAUAAGGCUAGUCCGUUAUCAACUUGAAA | |
| 31946039_spCas9 | AAGUGGCACCGAGUCGGUGCmUsmUsmUsU | |
| GuideDesign90_ABE_NGC | mAsmCsmAsAUCUGUGUUCUGGCACCGUUUUAGAGCUAGAA | 1164 |
| _20nt_3-9_008_−_ | AUAGCAAGUUAAAAUAAGGCUAGUCCGUUAUCAACUUGAAA | |
| 31946042_spCas9 | AAGUGGCACCGAGUCGGUGCmUsmUsmUSU | |
| GuideDesign90_ABE_NGC | mAsmCsmAsAUCUGUGUUCUGGCACCGUUUUAGAGCUAGAA | 1165 |
| _20nt_3-12_020_−_ | AUAGCAAGUUAAAAUAAGGCUAGUCCGUUAUCAACUUGAAA | |
| 31946042_spCas9 | AAGUGGCACCGAGUCGGUGCmUsmUsmUSU | |
| GuideDesign90_CBE_NGC | mAsmCsmAsAUCUGUGUUCUGGCACCGUUUUAGAGCUAGAA | 1166 |
| _20nt_4-9_009_−_ | AUAGCAAGUUAAAAUAAGGCUAGUCCGUUAUCAACUUGAAA | |
| 31946042_spCas9 | AAGUGGCACCGAGUCGGUGCmUsmUsmUSU | |
| GuideDesign90_ABE_NG | mAsmCsmAsAUCUGUGUUCUGGCACCGUUUUAGAGCUAGAA | 1167 |
| _20nt_3-9_027_−_ | AUAGCAAGUUAAAAUAAGGCUAGUCCGUUAUCAACUUGAAA | |
| 31946043_spCas9 | AAGUGGCACCGAGUCGGUGCmUsmUsmUsU | |
| GuideDesign90_CBE_NG | mAsmCsmAsAUCUGUGUUCUGGCACCGUUUUAGAGCUAGAA | 1168 |
| _20nt_4-9_028_−_ | AUAGCAAGUUAAAAUAAGGCUAGUCCGUUAUCAACUUGAAA | |
| 31946043_spCas9 | AAGUGGCACCGAGUCGGUGCmUsmUsmUSU | |
| GuideDesign90_ABE_NRN | mAsmCsmAsAUCUGUGUUCUGGCACCGUUUUAGAGCUAGAA | 1169 |
| _20nt_3-9_021_−_ | AUAGCAAGUUAAAAUAAGGCUAGUCCGUUAUCAACUUGAAA | |
| 31946042_spCas9 | AAGUGGCACCGAGUCGGUGCmUsmUsmUsU | |
| GuideDesign90_CBE_NRN | mAsmCsmAsAUCUGUGUUCUGGCACCGUUUUAGAGCUAGAA | 1170 |
| _20nt_3-9_022_−_ | AUAGCAAGUUAAAAUAAGGCUAGUCCGUUAUCAACUUGAAA | |
| 31946042_spCas9 | AAGUGGCACCGAGUCGGUGCmUsmUsmUSU | |
| GuideDesign90_MBE_NRN | mAsmCsmAsAUCUGUGUUCUGGCACCGUUUUAGAGCUAGAA | 1171 |
| _20nt_3-9_023_−_ | AUAGCAAGUUAAAAUAAGGCUAGUCCGUUAUCAACUUGAAA | |
| 31946042_spCas9 | AAGUGGCACCGAGUCGGUGCmUsmUsmUsU | |
| GuideDesign90_ABE_NGG | mAsmCsmAsCAGAUUGUAUAAAAGGCGUUUUAGAGCUAGAA | 1172 |
| _20nt_3- | AUAGCAAGUUAAAAUAAGGCUAGUCCGUUAUCAACUUGAAA | |
| 12_018_+_31946054_spCas9 | AAGUGGCACCGAGUCGGUGCmUsmUsmUsU | |
| GuideDesign90_ABE_NGG | mAsmCsmAsCAGAUUGUAUAAAAGGCGUUUUAGAGCUAGAA | 1173 |
| _20nt_13- | AUAGCAAGUUAAAAUAAGGCUAGUCCGUUAUCAACUUGAAA | |
| 16_032_+_31946054_SpCas | AAGUGGCACCGAGUCGGUGCmUsmUsmUSU | |
| 9 | | |
| GuideDesign90_CBE_NGG | mAsmCsmAsCAGAUUGUAUAAAAGGCGUUUUAGAGCUAGAA | 1174 |
| _20nt_4- | AUAGCAAGUUAAAAUAAGGCUAGUCCGUUAUCAACUUGAAA | |
| 9_003_+_31946054_spCas9 | AAGUGGCACCGAGUCGGUGCmUsmUsmUSU | |
| GuideDesign90_CBE_NGG | mAsmCsmAsCAGAUUGUAUAAAAGGCGUUUUAGAGCUAGAA | 1175 |
| _20nt_3- | AUAGCAAGUUAAAAUAAGGCUAGUCCGUUAUCAACUUGAAA | |
| 9_029_+_31946054_spCas9 | AAGUGGCACCGAGUCGGUGCmUsmUsmUsU | |
| GuideDesign90_CBE_NGG | mAsmCsmAsCAGAUUGUAUAAAAGGCGUUUUAGAGCUAGAA | 1176 |
| _20nt_3- | AUAGCAAGUUAAAAUAAGGCUAGUCCGUUAUCAACUUGAAA | |
| 16_033_+_31946054_SpCas | AAGUGGCACCGAGUCGGUGCmUsmUsmUsU | |
| 9 | | |
| GuideDesign90_ABE_NG | mAsmCsmAsCAGAUUGUAUAAAAGGCGUUUUAGAGCUAGAA | 1177 |
| _20nt_3- | AUAGCAAGUUAAAAUAAGGCUAGUCCGUUAUCAACUUGAAA | |
| 9_027_+_31946054_spCas9 | AAGUGGCACCGAGUCGGUGCmUsmUsmUSU | |
| GuideDesign90_CBE_NG | mAsmCsmAsCAGAUUGUAUAAAAGGCGUUUUAGAGCUAGAA | 1178 |
| _20nt_4- | AUAGCAAGUUAAAAUAAGGCUAGUCCGUUAUCAACUUGAAA | |
| 9_028_+_31946054_spCas9 | AAGUGGCACCGAGUCGGUGCmUsmUsmUsU | |
| GuideDesign90_ABE_NRN | mAsmCsmAsCAGAUUGUAUAAAAGGCGUUUUAGAGCUAGAA | 1179 |
| _20nt_3- | AUAGCAAGUUAAAAUAAGGCUAGUCCGUUAUCAACUUGAAA | |
| 9_021_+_31946054_spCas9 | AAGUGGCACCGAGUCGGUGCmUsmUsmUsU | |
| GuideDesign90_CBE_NRN | mAsmCsmAsCAGAUUGUAUAAAAGGCGUUUUAGAGCUAGAA | 1180 |
| _20nt_3- | AUAGCAAGUUAAAAUAAGGCUAGUCCGUUAUCAACUUGAAA | |
| 9_022_+_31946054_spCas9 | AAGUGGCACCGAGUCGGUGCmUsmUsmUsU | |
| Guide_Design90_MBE_NRN | mAsmCsmAsCAGAUUGUAUAAAAGGCGUUUUAGAGCUAGAA | 1181 |
| _20nt_3- | AUAGCAAGUUAAAAUAAGGCUAGUCCGUUAUCAACUUGAAA | |
| 9_023_+_31946054_spCas9 | AAGUGGCACCGAGUCGGUGCmUsmUsmUsU | |
| GuideDesign90_ABE_NGG | mAsmCsmAsGAUUGUAUAAAAGGCUGGUUUUAGAGCUAGAA | 1182 |
| _20nt_3- | AUAGCAAGUUAAAAUAAGGCUAGUCCGUUAUCAACUUGAAA | |
| 12_018_+_31946056_spCas9 | AAGUGGCACCGAGUCGGUGCmUsmUsmUSU | |
| GuideDesign90_ABE_NGG | mAsmCsmAsGAUUGUAUAAAAGGCUGGUUUUAGAGCUAGAA | 1183 |
| _20nt_13- | AUAGCAAGUUAAAAUAAGGCUAGUCCGUUAUCAACUUGAAA | |
| 16_032_+_31946056_SpCas | AAGUGGCACCGAGUCGGUGCmUsmUsmUsU | |
| 9 | | |
| GuideDesign90_CBE_NGG | mAsmCsmAsGAUUGUAUAAAAGGCUGGUUUUAGAGCUAGAA | 1184 |
| _20nt_4- | AUAGCAAGUUAAAAUAAGGCUAGUCCGUUAUCAACUUGAAA | |
| 9_003_+_31946056_spCas9 | AAGUGGCACCGAGUCGGUGCmUsmUsmUsU | |
| GuideDesign90_CBE_NGG | mAsmCsmAsGAUUGUAUAAAAGGCUGGUUUUAGAGCUAGAA | 1185 |
| _20nt_3- | AUAGCAAGUUAAAAUAAGGCUAGUCCGUUAUCAACUUGAAA | |
| 9_029_+_31946056_spCas9 | AAGUGGCACCGAGUCGGUGCmUsmUsmUsU | |
| GuideDesign90_CBE_NGG | mAsmCsmAsGAUUGUAUAAAAGGCUGGUUUUAGAGCUAGAA | 1186 |
| _20nt_3- | AUAGCAAGUUAAAAUAAGGCUAGUCCGUUAUCAACUUGAAA | |
| 16_033_+_31946056_SpCas | AAGUGGCACCGAGUCGGUGCmUsmUsmUsU | |
| 9 | | |
| GuideDesign90_ABE_NG | mAsmCsmAsGAUUGUAUAAAAGGCUGGUUUUAGAGCUAGAA | 1187 |
| _20nt_3- | AUAGCAAGUUAAAAUAAGGCUAGUCCGUUAUCAACUUGAAA | |
| 9_027_+_31946056_spCas9 | AAGUGGCACCGAGUCGGUGCmUsmUsmUsU | |
| GuideDesign90_CBE_NG | mAsmCsmAsGAUUGUAUAAAAGGCUGGUUUUAGAGCUAGAA | 1188 |
| _20nt_4- | AUAGCAAGUUAAAAUAAGGCUAGUCCGUUAUCAACUUGAAA | |
| 9_028_+_31946056_spCas9 | AAGUGGCACCGAGUCGGUGCmUsmUsmUSU | |
| GuideDesign90_ABE_NRN | mAsmCsmAsGAUUGUAUAAAAGGCUGGUUUUAGAGCUAGAA | 1189 |
| _20nt_3- | AUAGCAAGUUAAAAUAAGGCUAGUCCGUUAUCAACUUGAAA | |
| 9_021_+_31946056_spCas9 | AAGUGGCACCGAGUCGGUGCmUsmUsmUsU | |
| GuideDesign90_CBE_NRN | mAsmCsmAsGAUUGUAUAAAAGGCUGGUUUUAGAGCUAGAA | 1190 |
| _20nt_3- | AUAGCAAGUUAAAAUAAGGCUAGUCCGUUAUCAACUUGAAA | |
| 9_022_+_31946056_spCas9 | AAGUGGCACCGAGUCGGUGCmUsmUsmUsU | |
| GuideDesign90_MBE_NRN | mAsmCsmAsGAUUGUAUAAAAGGCUGGUUUUAGAGCUAGAA | 1191 |
| _20nt_3- | AUAGCAAGUUAAAAUAAGGCUAGUCCGUUAUCAACUUGAAA | |
| 9_023_+_31946056_spCas9 | AAGUGGCACCGAGUCGGUGCmUsmUsmUsU | |
| GuideDesign90_ABE_NGC | mAsmGsmAsACACAGAUUGUAUAAAAGUUUUAGAGCUAGAA | 1192 |
| _20nt_3- | AUAGCAAGUUAAAAUAAGGCUAGUCCGUUAUCAACUUGAAA | |
| 12_020_+_31946051_spCas9 | AAGUGGCACCGAGUCGGUGCmUsmUsmUsU | |
| GuideDesign90_CBE_NGC_ | mAsmGsmAsACACAGAUUGUAUAAAAGUUUUAGAGCUAGAA | 1193 |
| _20nt_4- | AUAGCAAGUUAAAAUAAGGCUAGUCCGUUAUCAACUUGAAA | |
| 9_009_+_31946051_spCas9 | AAGUGGCACCGAGUCGGUGCmUsmUsmUsU | |
| GuideDesign90_ABE_NG | mAsmGsmAsACACAGAUUGUAUAAAAGUUUUAGAGCUAGAA | 1194 |
| _20nt_3- | AUAGCAAGUUAAAAUAAGGCUAGUCCGUUAUCAACUUGAAA | |
| 9_027_+_31946051_spCas9 | AAGUGGCACCGAGUCGGUGCmUsmUsmUsU | |
| GuideDesign90_CBE_NG | mAsmGsmAsACACAGAUUGUAUAAAAGUUUUAGAGCUAGAA | 1195 |
| _20nt_4- | AUAGCAAGUUAAAAUAAGGCUAGUCCGUUAUCAACUUGAAA | |
| 9_028_+_31946051_spCas9 | AAGUGGCACCGAGUCGGUGCmUsmUsmUsU | |
| GuideDesign90_ABE_NRN | mAsmGsmAsACACAGAUUGUAUAAAAGUUUUAGAGCUAGAA | 1196 |
| _20nt_3- | AUAGCAAGUUAAAAUAAGGCUAGUCCGUUAUCAACUUGAAA | |
| 9_021_+_31946051_spCas9 | AAGUGGCACCGAGUCGGUGCmUsmUsmUsU | |
| GuideDesign90_CBE_NRN | mAsmGsmAsACACAGAUUGUAUAAAAGUUUUAGAGCUAGAA | 1197 |
| _20nt_3- | AUAGCAAGUUAAAAUAAGGCUAGUCCGUUAUCAACUUGAAA | |
| 9_022_+_31946051_spCas9 | AAGUGGCACCGAGUCGGUGCmUsmUsmUsU | |
| GuideDesign90_MBE_NRN | mAsmGsmAsACACAGAUUGUAUAAAAGUUUUAGAGCUAGAA | 1198 |
| _20nt_3- | AUAGCAAGUUAAAAUAAGGCUAGUCCGUUAUCAACUUGAAA | |
| 9_023_+_31946051_spCas9 | AAGUGGCACCGAGUCGGUGCmUsmUsmUsU | |
| GuideDesign90_ABE_NRCH | mAsmGsmAsACACAGAUUGUAUAAAAGUUUUAGAGCUAGAA | 1199 |
| _20nt_3- | AUAGCAAGUUAAAAUAAGGCUAGUCCGUUAUCAACUUGAAA | |
| 9_024_+_31946051_spCas9 | AAGUGGCACCGAGUCGGUGCmUsmUsmUsU | |
| GuideDesign90_CBE_NRCH | mAsmGsmAsACACAGAUUGUAUAAAAGUUUUAGAGCUAGAA | 1200 |
| _20nt_3- | AUAGCAAGUUAAAAUAAGGCUAGUCCGUUAUCAACUUGAAA | |
| 9_030_+_31946051_spCas9 | AAGUGGCACCGAGUCGGUGCmUsmUsmUSU | |
| GuideDesign90_ABE_NGC | mAsmGsmAsUUGUAUAAAAGGCUGGGGUUUUAGAGCUAGAA | 1201 |
| _20nt_3- | AUAGCAAGUUAAAAUAAGGCUAGUCCGUUAUCAACUUGAAA | |
| 9_008_+_31946058_spCas9 | AAGUGGCACCGAGUCGGUGCmUsmUsmUSU | |
| GuideDesign90_ABE_NGC | mAsmGsmAsUUGUAUAAAAGGCUGGGGUUUUAGAGCUAGAA | 1202 |
| _20nt_3- | AUAGCAAGUUAAAAUAAGGCUAGUCCGUUAUCAACUUGAAA | |
| 12_020_+_31946058_spCas9 | AAGUGGCACCGAGUCGGUGCmUsmUsmUSU | |
| GuideDesign90_CBE_NGC | mAsmGsmAsUUGUAUAAAAGGCUGGGGUUUUAGAGCUAGAA | 1203 |
| _20nt_4- | AUAGCAAGUUAAAAUAAGGCUAGUCCGUUAUCAACUUGAAA | |
| 9_009_+_31946058_spCas9 | AAGUGGCACCGAGUCGGUGCmUsmUsmUsU | |
| GuideDesign90_ABE_NG | mAsmGsmAsUUGUAUAAAAGGCUGGGGUUUUAGAGCUAGAA | 1204 |
| _20nt_3- | AUAGCAAGUUAAAAUAAGGCUAGUCCGUUAUCAACUUGAAA | |
| 9_027_+_31946058_spCas9 | AAGUGGCACCGAGUCGGUGCmUsmUsmUSU | |
| GuideDesign90_CBE_NG | mAsmGsmAsUUGUAUAAAAGGCUGGGGUUUUAGAGCUAGAA | 1205 |
| _20nt_4- | AUAGCAAGUUAAAAUAAGGCUAGUCCGUUAUCAACUUGAAA | |
| 9_028_+_31946058_spCas9 | AAGUGGCACCGAGUCGGUGCmUsmUsmUsU | |
| GuideDesign90_ABE_NRN | mAsmGsmAsUUGUAUAAAAGGCUGGGGUUUUAGAGCUAGAA | 1206 |
| _20nt_3- | AUAGCAAGUUAAAAUAAGGCUAGUCCGUUAUCAACUUGAAA | |
| 9_021_+_31946058_spCas9 | AAGUGGCACCGAGUCGGUGCmUsmUsmUsU | |
| GuideDesign90_CBE_NRN | mAsmGsmAsUUGUAUAAAAGGCUGGGGUUUUAGAGCUAGAA | 1207 |
| _20nt_3- | AUAGCAAGUUAAAAUAAGGCUAGUCCGUUAUCAACUUGAAA | |
| 9_022_+_31946058_spCas9 | AAGUGGCACCGAGUCGGUGCmUsmUsmUSU | |
| GuideDesign90_MBE_NRN | mAsmGsmAsUUGUAUAAAAGGCUGGGGUUUUAGAGCUAGAA | 1208 |
| _20nt_3- | AUAGCAAGUUAAAAUAAGGCUAGUCCGUUAUCAACUUGAAA | |
| 9_023_+_31946058_spCas9 | AAGUGGCACCGAGUCGGUGCmUsmUsmUsU | |
| GuideDesign90_ABE_NRCH | mAsmGsmAsUUGUAUAAAAGGCUGGGGUUUUAGAGCUAGAA | 1209 |
| _20nt_3- | AUAGCAAGUUAAAAUAAGGCUAGUCCGUUAUCAACUUGAAA | |
| 9_024_+_31946058_spCas9 | AAGUGGCACCGAGUCGGUGCmUsmUsmUsU | |
| GuideDesign90_CBE_NRCH | mAsmGsmAsUUGUAUAAAAGGCUGGGGUUUUAGAGCUAGAA | 1210 |
| _20nt_3- | AUAGCAAGUUAAAAUAAGGCUAGUCCGUUAUCAACUUGAAA | |
| 9_030_+_31946058_spCas9 | AAGUGGCACCGAGUCGGUGCmUsmUsmUsU | |
| GuideDesign90_CBE_NNNR | mAsmGsmAsUUGUAUAAAAGGCUGGGGGUUUUAGUACUCUG | 1211 |
| RT_21nt_3- | UAAUGAAAAUUACAGAAUCUACUAAAACAAGGCAAAAUGCC | |
| 12_015_+_31946058_saCas9 | GUGUUUAUCUCGUCAACUUGUUGGCGAGAUsmUsmUsmU | |
| GuideDesign90_ABE_NGG | mAsmUsmAsAAAGGCUGGGGGCUGGUGUUUUAGAGCUAGAA | 1212 |
| _20nt_3- | AUAGCAAGUUAAAAUAAGGCUAGUCCGUUAUCAACUUGAAA | |
| 12_018_+_31946065_spCas9 | AAGUGGCACCGAGUCGGUGCmUsmUsmUSU | |
| GuideDesign90_CBE_NGG | mAsmUsmAsAAAGGCUGGGGGCUGGUGUUUUAGAGCUAGAA | 1213 |
| _20nt_4- | AUAGCAAGUUAAAAUAAGGCUAGUCCGUUAUCAACUUGAAA | |
| 9_003_+_31946065_spCas9 | AAGUGGCACCGAGUCGGUGCmUsmUsmUsU | |
| GuideDesign90_CBE_NGG | mAsmUsmAsAAAGGCUGGGGGCUGGUGUUUUAGAGCUAGAA | 1214 |
| _20nt_3- | AUAGCAAGUUAAAAUAAGGCUAGUCCGUUAUCAACUUGAAA | |
| 9_029_+_31946065_spCas9 | AAGUGGCACCGAGUCGGUGCmUsmUsmUsU | |
| GuideDesign90_CBE_NGG | mAsmUsmAsAAAGGCUGGGGGCUGGUGUUUUAGAGCUAGAA | 1215 |
| _20nt_3- | AUAGCAAGUUAAAAUAAGGCUAGUCCGUUAUCAACUUGAAA | |
| 16_033_+_31946065_SpCas | AAGUGGCACCGAGUCGGUGCmUsmUsmUsU | |
| 9 | | |
| GuideDesign90_ABE_NG | mAsmUsmAsAAAGGCUGGGGGCUGGUGUUUUAGAGCUAGAA | 1216 |
| _20nt_3- | AUAGCAAGUUAAAAUAAGGCUAGUCCGUUAUCAACUUGAAA | |
| 9_027_+_31946065_spCas9 | AAGUGGCACCGAGUCGGUGCmUsmUsmUsU | |
| GuideDesign90_CBE_NG | mAsmUsmAsAAAGGCUGGGGGCUGGUGUUUUAGAGCUAGAA | 1217 |
| _20nt_4- | AUAGCAAGUUAAAAUAAGGCUAGUCCGUUAUCAACUUGAAA | |
| 9_028_+_31946065_spCas9 | AAGUGGCACCGAGUCGGUGCmUsmUsmUsU | |
| GuideDesign90_ABE_NRN | mAsmUsmAsAAAGGCUGGGGGCUGGUGUUUUAGAGCUAGAA | 1218 |
| _20nt_3- | AUAGCAAGUUAAAAUAAGGCUAGUCCGUUAUCAACUUGAAA | |
| 9_021_+_31946065_spCas9 | AAGUGGCACCGAGUCGGUGCmUsmUsmUsU | |
| GuideDesign90_CBE_NRN | mAsmUsmAsAAAGGCUGGGGGCUGGUGUUUUAGAGCUAGAA | 1219 |
| _20nt_3- | AUAGCAAGUUAAAAUAAGGCUAGUCCGUUAUCAACUUGAAA | |
| 9_022_+_31946065_spCas9 | AAGUGGCACCGAGUCGGUGCmUsmUsmUSU | |
| GuideDesign90_MBE_NRN | mAsmUsmAsAAAGGCUGGGGGCUGGUGUUUUAGAGCUAGAA | 1220 |
| _20nt_3- | AUAGCAAGUUAAAAUAAGGCUAGUCCGUUAUCAACUUGAAA | |
| 9_023_+_31946065_spCas9 | AAGUGGCACCGAGUCGGUGCmUsmUsmUsU | |
| GuideDesign90_ABE_NGC | mAsmUsmCsUGUGUUCUGGCACCUGCGUUUUAGAGCUAGAA | 1221 |
| _20nt_3-12_020_−_ | AUAGCAAGUUAAAAUAAGGCUAGUCCGUUAUCAACUUGAAA | |
| 31946039_spCas9 | AAGUGGCACCGAGUCGGUGCmUsmUsmUSU | |
| GuideDesign90_CBE_NGC | mAsmUsmCsUGUGUUCUGGCACCUGCGUUUUAGAGCUAGAA | 1222 |
| _20nt_4-9_009_−_ | AUAGCAAGUUAAAAUAAGGCUAGUCCGUUAUCAACUUGAAA | |
| 31946039_spCas9 | AAGUGGCACCGAGUCGGUGCmUsmUsmUsU | |
| GuideDesign90_ABE_NG | mAsmUsmCsUGUGUUCUGGCACCUGCGUUUUAGAGCUAGAA | 1223 |
| _20nt_3-9_027_−_ | AUAGCAAGUUAAAAUAAGGCUAGUCCGUUAUCAACUUGAAA | |
| 31946040_spCas9 | AAGUGGCACCGAGUCGGUGCmUsmUsmUSU | |
| GuideDesign90_CBE_NG | mAsmUsmCsUGUGUUCUGGCACCUGCGUUUUAGAGCUAGAA | 1224 |
| _20nt_4-9_028_−_ | AUAGCAAGUUAAAAUAAGGCUAGUCCGUUAUCAACUUGAAA | |
| 31946040_spCas9 | AAGUGGCACCGAGUCGGUGCmUsmUsmUsU | |
| GuideDesign90_ABE_NRN | mAsmUsmCsUGUGUUCUGGCACCUGCGUUUUAGAGCUAGAA | 1225 |
| _20nt_3-9_021_−_ | AUAGCAAGUUAAAAUAAGGCUAGUCCGUUAUCAACUUGAAA | |
| 31946039_spCas9 | AAGUGGCACCGAGUCGGUGCmUsmUsmUsU | |
| GuideDesign90_CBE_NRN | mAsmUsmCsUGUGUUCUGGCACCUGCGUUUUAGAGCUAGAA | 1226 |
| _20nt_3-9_022_−_ | AUAGCAAGUUAAAAUAAGGCUAGUCCGUUAUCAACUUGAAA | |
| 31946039_spCas9 | AAGUGGCACCGAGUCGGUGCmUsmUsmUsU | |
| GuideDesign90_MBE_NRN | mAsmUsmCsUGUGUUCUGGCACCUGCGUUUUAGAGCUAGAA | 1227 |
| _20nt_3-9_023_−_ | AUAGCAAGUUAAAAUAAGGCUAGUCCGUUAUCAACUUGAAA | |
| 31946039_spCas9 | AAGUGGCACCGAGUCGGUGCmUsmUsmUsU | |
| GuideDesign90_ABE_NRCH | mAsmUsmCsUGUGUUCUGGCACCUGCGUUUUAGAGCUAGAA | 1228 |
| _20nt_3-9_024_−_ | AUAGCAAGUUAAAAUAAGGCUAGUCCGUUAUCAACUUGAAA | |
| 31946038_spCas9 | AAGUGGCACCGAGUCGGUGCmUsmUsmUsU | |
| GuideDesign90_CBE_NRCH | mAsmUsmCsUGUGUUCUGGCACCUGCGUUUUAGAGCUAGAA | 1229 |
| _20nt_3-9_030_−_ | AUAGCAAGUUAAAAUAAGGCUAGUCCGUUAUCAACUUGAAA | |
| 31946038_spCas9 | AAGUGGCACCGAGUCGGUGCmUsmUsmUsU | |
| GuideDesign90_ABE_NGG | mCsmAsmCsAGAUUGUAUAAAAGGCUGUUUUAGAGCUAGAA | 1230 |
| _20nt_3- | AUAGCAAGUUAAAAUAAGGCUAGUCCGUUAUCAACUUGAAA | |
| 12_018_+_31946055_spCas9 | AAGUGGCACCGAGUCGGUGCmUsmUsmUsU | |
| GuideDesign90_ABE_NGG | mCsmAsmCsAGAUUGUAUAAAAGGCUGUUUUAGAGCUAGAA | 1231 |
| _20nt_13- | AUAGCAAGUUAAAAUAAGGCUAGUCCGUUAUCAACUUGAAA | |
| 16_032_+_31946055_SpCas | AAGUGGCACCGAGUCGGUGCmUsmUsmUsU | |
| 9 | | |
| GuideDesign90_CBE_NGG | mCsmAsmCsAGAUUGUAUAAAAGGCUGUUUUAGAGCUAGAA | 1232 |
| _20nt_4- | AUAGCAAGUUAAAAUAAGGCUAGUCCGUUAUCAACUUGAAA | |
| 9_003_+_31946055_spCas9 | AAGUGGCACCGAGUCGGUGCmUsmUsmUsU | |
| GuideDesign90_CBE_NGG | mCsmAsmCsAGAUUGUAUAAAAGGCUGUUUUAGAGCUAGAA | 1233 |
| _20nt_3- | AUAGCAAGUUAAAAUAAGGCUAGUCCGUUAUCAACUUGAAA | |
| 9_029_+_31946055_spCas9 | AAGUGGCACCGAGUCGGUGCmUsmUsmUSU | |
| GuideDesign90_CBE_NGG | mCsmAsmCsAGAUUGUAUAAAAGGCUGUUUUAGAGCUAGAA | 1234 |
| _20nt_3- | AUAGCAAGUUAAAAUAAGGCUAGUCCGUUAUCAACUUGAAA | |
| 16_033_+_31946055_SpCas | AAGUGGCACCGAGUCGGUGCmUsmUsmUsU | |
| 9 | | |
| GuideDesign90_ABE_NG | mCsmAsmCsAGAUUGUAUAAAAGGCUGUUUUAGAGCUAGAA | 1235 |
| _20nt_3- | AUAGCAAGUUAAAAUAAGGCUAGUCCGUUAUCAACUUGAAA | |
| 9_027_+_31946055_spCas9 | AAGUGGCACCGAGUCGGUGCmUsmUsmUsU | |
| GuideDesign90_CBE_NG | mCsmAsmCsAGAUUGUAUAAAAGGCUGUUUUAGAGCUAGAA | 1236 |
| _20nt_4- | AUAGCAAGUUAAAAUAAGGCUAGUCCGUUAUCAACUUGAAA | |
| 9_028_+_31946055_spCas9 | AAGUGGCACCGAGUCGGUGCmUsmUsmUsU | |
| GuideDesign90_ABE_NRN | mCsmAsmCsAGAUUGUAUAAAAGGCUGUUUUAGAGCUAGAA | 1237 |
| _20nt_3- | AUAGCAAGUUAAAAUAAGGCUAGUCCGUUAUCAACUUGAAA | |
| 9_021_+_31946055_spCas9 | AAGUGGCACCGAGUCGGUGCmUsmUsmUsU | |
| GuideDesign90_CBE_NRN | mCsmAsmCsAGAUUGUAUAAAAGGCUGUUUUAGAGCUAGAA | 1238 |
| _20nt_3- | AUAGCAAGUUAAAAUAAGGCUAGUCCGUUAUCAACUUGAAA | |
| 9_022_+_31946055_spCas9 | AAGUGGCACCGAGUCGGUGCmUsmUsmUsU | |
| GuideDesign90_MBE_NRN | mCsmAsmCsAGAUUGUAUAAAAGGCUGUUUUAGAGCUAGAA | 1239 |
| _20nt_3- | AUAGCAAGUUAAAAUAAGGCUAGUCCGUUAUCAACUUGAAA | |
| 9_023_+_31946055_spCas9 | AAGUGGCACCGAGUCGGUGCmUsmUsmUSU | |
| GuideDesign90_ABE_NGG | mCsmAsmGsAACACAGAUUGUAUAAAGUUUUAGAGCUAGAA | 1240 |
| _20nt_3- | AUAGCAAGUUAAAAUAAGGCUAGUCCGUUAUCAACUUGAAA | |
| 12_018_+_31946050_spCas9 | AAGUGGCACCGAGUCGGUGCmUsmUsmUsU | |
| GuideDesign90_ABE_NGG | mCsmAsmGsAACACAGAUUGUAUAAAGUUUUAGAGCUAGAA | 1241 |
| _20nt_13- | AUAGCAAGUUAAAAUAAGGCUAGUCCGUUAUCAACUUGAAA | |
| 16_032_+_31946050_SpCas | AAGUGGCACCGAGUCGGUGCmUsmUsmUsU | |
| 9 | | |
| GuideDesign90_CBE_NGG | mCsmAsmGsAACACAGAUUGUAUAAAGUUUUAGAGCUAGAA | 1242 |
| _20nt_4- | AUAGCAAGUUAAAAUAAGGCUAGUCCGUUAUCAACUUGAAA | |
| 9_003_+_31946050_spCas9 | AAGUGGCACCGAGUCGGUGCmUsmUsmUsU | |
| GuideDesign90_CBE_NGG | mCsmAsmGsAACACAGAUUGUAUAAAGUUUUAGAGCUAGAA | 1243 |
| _20nt_3- | AUAGCAAGUUAAAAUAAGGCUAGUCCGUUAUCAACUUGAAA | |
| 9_029_+_31946050_spCas9 | AAGUGGCACCGAGUCGGUGCmUsmUsmUsU | |
| GuideDesign90_CBE_NGG | mCsmAsmGsAACACAGAUUGUAUAAAGUUUUAGAGCUAGAA | 1244 |
| _20nt_3- | AUAGCAAGUUAAAAUAAGGCUAGUCCGUUAUCAACUUGAAA | |
| 16_033_+_31946050_SpCas | AAGUGGCACCGAGUCGGUGCmUsmUsmUsU | |
| 9 | | |
| GuideDesign90_ABE_NG | mCsmAsmGsAACACAGAUUGUAUAAAGUUUUAGAGCUAGAA | 1245 |
| _20nt_3- | AUAGCAAGUUAAAAUAAGGCUAGUCCGUUAUCAACUUGAAA | |
| 9_027_+_31946050_spCas9 | AAGUGGCACCGAGUCGGUGCmUsmUsmUsU | |
| GuideDesign90_CBE_NG | mCsmAsmGsAACACAGAUUGUAUAAAGUUUUAGAGCUAGAA | 1246 |
| _20nt_4- | AUAGCAAGUUAAAAUAAGGCUAGUCCGUUAUCAACUUGAAA | |
| 9_028_+_31946050_spCas9 | AAGUGGCACCGAGUCGGUGCmUsmUsmUsU | |
| GuideDesign90_ABE_NRN | mCsmAsmGsAACACAGAUUGUAUAAAGUUUUAGAGCUAGAA | 1247 |
| _20nt_3- | AUAGCAAGUUAAAAUAAGGCUAGUCCGUUAUCAACUUGAAA | |
| 9_021_+_31946050_spCas9 | AAGUGGCACCGAGUCGGUGCmUsmUsmUsU | |
| GuideDesign90_CBE_NRN | mCsmAsmGsAACACAGAUUGUAUAAAGUUUUAGAGCUAGAA | 1248 |
| _20nt_3- | AUAGCAAGUUAAAAUAAGGCUAGUCCGUUAUCAACUUGAAA | |
| 9_022_+_31946050_spCas9 | AAGUGGCACCGAGUCGGUGCmUsmUsmUsU | |
| GuideDesign90_MBE_NRN | mCsmAsmGsAACACAGAUUGUAUAAAGUUUUAGAGCUAGAA | 1249 |
| _20nt_3- | AUAGCAAGUUAAAAUAAGGCUAGUCCGUUAUCAACUUGAAA | |
| 9_023_+_31946050_spCas9 | AAGUGGCACCGAGUCGGUGCmUsmUsmUsU | |
| GuideDesign90_CBE_NRRH | mCsmAsmGsAACACAGAUUGUAUAAAGUUUUAGAGCUAGAA | 1250 |
| _20nt_3- | AUAGCAAGUUAAAAUAAGGCUAGUCCGUUAUCAACUUGAAA | |
| 9_031_+_31946050_spCas9 | AAGUGGCACCGAGUCGGUGCmUsmUsmUsU | |
| GuideDesign90_ABE_NGG | mCsmAsmGsAUUGUAUAAAAGGCUGGGUUUUAGAGCUAGAA | 1251 |
| _20nt_3- | AUAGCAAGUUAAAAUAAGGCUAGUCCGUUAUCAACUUGAAA | |
| 12_018_+_31946057_spCas9 | AAGUGGCACCGAGUCGGUGCmUsmUsmUSU | |
| GuideDesign90_ABE_NGG | mCsmAsmGsAUUGUAUAAAAGGCUGGGUUUUAGAGCUAGAA | 1252 |
| _20nt_13- | AUAGCAAGUUAAAAUAAGGCUAGUCCGUUAUCAACUUGAAA | |
| 16_032_+_31946057_SpCas | AAGUGGCACCGAGUCGGUGCmUsmUsmUsU | |
| 9 | | |
| GuideDesign90_CBE_NGG | mCsmAsmGsAUUGUAUAAAAGGCUGGGUUUUAGAGCUAGAA | 1253 |
| _20nt_4- | AUAGCAAGUUAAAAUAAGGCUAGUCCGUUAUCAACUUGAAA | |
| 9_003_+_31946057_spCas9 | AAGUGGCACCGAGUCGGUGCmUsmUsmUsU | |
| GuideDesign90_CBE_NGG | mCsmAsmGsAUUGUAUAAAAGGCUGGGUUUUAGAGCUAGAA | 1254 |
| _20nt_3- | AUAGCAAGUUAAAAUAAGGCUAGUCCGUUAUCAACUUGAAA | |
| 9_029_+_31946057_spCas9 | AAGUGGCACCGAGUCGGUGCmUsmUsmUSU | |
| GuideDesign90_CBE_NGG | mCsmAsmGsAUUGUAUAAAAGGCUGGGUUUUAGAGCUAGAA | 1255 |
| _20nt_3- | AUAGCAAGUUAAAAUAAGGCUAGUCCGUUAUCAACUUGAAA | |
| 16_033_+_31946057_SpCas | AAGUGGCACCGAGUCGGUGCmUsmUsmUsU | |
| 9 | | |
| GuideDesign90_ABE_NG | mCsmAsmGsAUUGUAUAAAAGGCUGGGUUUUAGAGCUAGAA | 1256 |
| _20nt_3- | AUAGCAAGUUAAAAUAAGGCUAGUCCGUUAUCAACUUGAAA | |
| 9_027_+_31946057_spCas9 | AAGUGGCACCGAGUCGGUGCmUsmUsmUsU | |
| GuideDesign90_CBE_NG | mCsmAsmGsAUUGUAUAAAAGGCUGGGUUUUAGAGCUAGAA | 1257 |
| _20nt_4- | AUAGCAAGUUAAAAUAAGGCUAGUCCGUUAUCAACUUGAAA | |
| 9_028_+_31946057_spCas9 | AAGUGGCACCGAGUCGGUGCmUsmUsmUsU | |
| GuideDesign90_ABE_NRN | mCsmAsmGsAUUGUAUAAAAGGCUGGGUUUUAGAGCUAGAA | 1258 |
| _20nt_3- | AUAGCAAGUUAAAAUAAGGCUAGUCCGUUAUCAACUUGAAA | |
| 9_021_+_31946057_spCas9 | AAGUGGCACCGAGUCGGUGCmUsmUsmUsU | |
| GuideDesign90_CBE_NRN | mCsmAsmGsAUUGUAUAAAAGGCUGGGUUUUAGAGCUAGAA | 1259 |
| _20nt_3- | AUAGCAAGUUAAAAUAAGGCUAGUCCGUUAUCAACUUGAAA | |
| 9_022_+_31946057_spCas9 | AAGUGGCACCGAGUCGGUGCmUsmUsmUsU | |
| GuideDesign90_MBE_NRN | mCsmAsmGsAUUGUAUAAAAGGCUGGGUUUUAGAGCUAGAA | 1260 |
| _20nt_3- | AUAGCAAGUUAAAAUAAGGCUAGUCCGUUAUCAACUUGAAA | |
| 9_023_+_31946057_spCas9 | AAGUGGCACCGAGUCGGUGCmUsmUsmUsU | |
| GuideDesign90_CBE_NRRH | mCsmAsmGsAUUGUAUAAAAGGCUGGGUUUUAGAGCUAGAA | 1261 |
| _20nt_3- | AUAGCAAGUUAAAAUAAGGCUAGUCCGUUAUCAACUUGAAA | |
| 9_031_+_31946057_spCas9 | AAGUGGCACCGAGUCGGUGCmUsmUsmUsU | |
| GuideDesign90_ABE_NRN | mCsmAsmGsCCCCCAGCCUUUUAUACGUUUUAGAGCUAGAA | 1262 |
| _20nt_3-9_021_−_ | AUAGCAAGUUAAAAUAAGGCUAGUCCGUUAUCAACUUGAAA | |
| 31946060_spCas9 | AAGUGGCACCGAGUCGGUGCmUsmUsmUsU | |
| GuideDesign90_CBE_NRN | mCsmAsmGsCCCCCAGCCUUUUAUACGUUUUAGAGCUAGAA | 1263 |
| _20nt_3-9_022_−_ | AUAGCAAGUUAAAAUAAGGCUAGUCCGUUAUCAACUUGAAA | |
| 31946060_spCas9 | AAGUGGCACCGAGUCGGUGCmUsmUsmUsU | |
| GuideDesign90_MBE_NRN | mCsmAsmGsCCCCCAGCCUUUUAUACGUUUUAGAGCUAGAA | 1264 |
| _20nt_3-9_023_−_ | AUAGCAAGUUAAAAUAAGGCUAGUCCGUUAUCAACUUGAAA | |
| 31946060_spCas9 | AAGUGGCACCGAGUCGGUGCmUsmUsmUsU | |
| GuideDesign90_ABE_NRTH | mCsmAsmGsCCCCCAGCCUUUUAUACGUUUUAGAGCUAGAA | 1265 |
| _20nt_3-9_025_−_ | AUAGCAAGUUAAAAUAAGGCUAGUCCGUUAUCAACUUGAAA | |
| 31946059_spCas9 | AAGUGGCACCGAGUCGGUGCmUsmUsmUsU | |
| GuideDesign90_CBE_NRTH | mCsmAsmGsCCCCCAGCCUUUUAUACGUUUUAGAGCUAGAA | 1266 |
| _20nt_4-9_026_−_ | AUAGCAAGUUAAAAUAAGGCUAGUCCGUUAUCAACUUGAAA | |
| 31946059_spCas9 | AAGUGGCACCGAGUCGGUGCmUsmUsmUsU | |
| GuideDesign90_CBE_NNNR | mCsmCsmAsCCAGCCCCCAGCCUUUUAGUUUUAGUACUCUG | 1267 |
| RT_21nt_3-12_015_−_ | UAAUGAAAAUUACAGAAUCUACUAAAACAAGGCAAAAUGCC | |
| 31946060_saCas9 | GUGUUUAUCUCGUCAACUUGUUGGCGAGAUsmUsmUsmU | |
| GuideDesign90_ABE_NRN | mCsmCsmAsGCCCCCAGCCUUUUAUAGUUUUAGAGCUAGAA | 1268 |
| _20nt_3-9_021_−_ | AUAGCAAGUUAAAAUAAGGCUAGUCCGUUAUCAACUUGAAA | |
| 31946061_spCas9 | AAGUGGCACCGAGUCGGUGCmUsmUsmUsU | |
| GuideDesign90_CBE_NRN | mCsmCsmAsGCCCCCAGCCUUUUAUAGUUUUAGAGCUAGAA | 1269 |
| _20nt_3-9_022_−_ | AUAGCAAGUUAAAAUAAGGCUAGUCCGUUAUCAACUUGAAA | |
| 31946061_spCas9 | AAGUGGCACCGAGUCGGUGCmUsmUsmUsU | |
| GuideDesign90_MBE_NRN | mCsmCsmAsGCCCCCAGCCUUUUAUAGUUUUAGAGCUAGAA | 1270 |
| _20nt_3-9_023_−_ | AUAGCAAGUUAAAAUAAGGCUAGUCCGUUAUCAACUUGAAA | |
| 31946061_spCas9 | AAGUGGCACCGAGUCGGUGCmUsmUsmUsU | |
| GuideDesign90_CBE_NRRH | mCsmCsmAsGCCCCCAGCCUUUUAUAGUUUUAGAGCUAGAA | 1271 |
| _20nt_3-9_031_−_ | AUAGCAAGUUAAAAUAAGGCUAGUCCGUUAUCAACUUGAAA | |
| 31946060_spCas9 | AAGUGGCACCGAGUCGGUGCmUsmUsmUsU | |
| GuideDesign90_ABE_NG | mCsmCsmAsGCCUUUUAUACAAUCUGGUUUUAGAGCUAGAA | 1272 |
| _20nt_3-9_027_−_ | AUAGCAAGUUAAAAUAAGGCUAGUCCGUUAUCAACUUGAAA | |
| 31946055_spCas9 | AAGUGGCACCGAGUCGGUGCmUsmUsmUsU | |
| GuideDesign90_CBE_NG | mCsmCsmAsGCCUUUUAUACAAUCUGGUUUUAGAGCUAGAA | 1273 |
| _20nt_4-9_028_−_ | AUAGCAAGUUAAAAUAAGGCUAGUCCGUUAUCAACUUGAAA | |
| 31946055_spCas9 | AAGUGGCACCGAGUCGGUGCmUsmUsmUsU | |
| GuideDesign90_ABE_NRN | mCsmCsmAsGCCUUUUAUACAAUCUGGUUUUAGAGCUAGAA | 1274 |
| _20nt_3-9_021_−_ | AUAGCAAGUUAAAAUAAGGCUAGUCCGUUAUCAACUUGAAA | |
| 31946054_spCas9 | AAGUGGCACCGAGUCGGUGCmUsmUsmUsU | |
| GuideDesign90_CBE_NRN | mCsmCsmAsGCCUUUUAUACAAUCUGGUUUUAGAGCUAGAA | 1275 |
| _20nt_3-9_022_−_ | AUAGCAAGUUAAAAUAAGGCUAGUCCGUUAUCAACUUGAAA | |
| 31946054_spCas9 | AAGUGGCACCGAGUCGGUGCmUsmUsmUsU | |
| GuideDesign90_MBE_NRN | mCsmCsmAsGCCUUUUAUACAAUCUGGUUUUAGAGCUAGAA | 1276 |
| _20nt_3-9_023_−_ | AUAGCAAGUUAAAAUAAGGCUAGUCCGUUAUCAACUUGAAA | |
| 31946054_spCas9 | AAGUGGCACCGAGUCGGUGCmUsmUsmUsU | |
| GuideDesign90_ABE_NRTH | mCsmCsmAsGCCUUUUAUACAAUCUGGUUUUAGAGCUAGAA | 1277 |
| _20nt_3-9_025_−_ | AUAGCAAGUUAAAAUAAGGCUAGUCCGUUAUCAACUUGAAA | |
| 31946053_spCas9 | AAGUGGCACCGAGUCGGUGCmUsmUsmUsU | |
| GuideDesign90_CBE_NRTH | mCsmCsmAsGCCUUUUAUACAAUCUGGUUUUAGAGCUAGAA | 1278 |
| _20nt_4-9_026_−_ | AUAGCAAGUUAAAAUAAGGCUAGUCCGUUAUCAACUUGAAA | |
| 31946053_spCas9 | AAGUGGCACCGAGUCGGUGCmUsmUsmUsU | |
| GuideDesign90_ABE_NG | mCsmCsmCsCAGCCUUUUAUACAAUCGUUUUAGAGCUAGAA | 1279 |
| _20nt_3-9_027_−_ | AUAGCAAGUUAAAAUAAGGCUAGUCCGUUAUCAACUUGAAA | |
| 31946057_spCas9 | AAGUGGCACCGAGUCGGUGCmUsmUsmUsU | |
| GuideDesign90_CBE_NG | mCsmCsmCsCAGCCUUUUAUACAAUCGUUUUAGAGCUAGAA | 1280 |
| _20nt_4-9_028_−_ | AUAGCAAGUUAAAAUAAGGCUAGUCCGUUAUCAACUUGAAA | |
| 31946057_spCas9 | AAGUGGCACCGAGUCGGUGCmUsmUsmUsU | |
| GuideDesign90_ABE_NRN | mCsmCsmCsCAGCCUUUUAUACAAUCGUUUUAGAGCUAGAA | 1281 |
| _20nt_3-9_021_−_ | AUAGCAAGUUAAAAUAAGGCUAGUCCGUUAUCAACUUGAAA | |
| 31946056_spCas9 | AAGUGGCACCGAGUCGGUGCmUsmUsmUsU | |
| GuideDesign90_CBE_NRN | mCsmCsmCsCAGCCUUUUAUACAAUCGUUUUAGAGCUAGAA | 1282 |
| _20nt_3-9_022_−_ | AUAGCAAGUUAAAAUAAGGCUAGUCCGUUAUCAACUUGAAA | |
| 31946056_spCas9 | AAGUGGCACCGAGUCGGUGCmUsmUsmUsU | |
| GuideDesign90_MBE_NRN | mCsmCsmCsCAGCCUUUUAUACAAUCGUUUUAGAGCUAGAA | 1283 |
| _20nt_3-9_023_−_ | AUAGCAAGUUAAAAUAAGGCUAGUCCGUUAUCAACUUGAAA | |
| 31946056_spCas9 | AAGUGGCACCGAGUCGGUGCmUsmUsmUsU | |
| GuideDesign90_ABE_NGG | mCsmUsmGsGGGGCUGGUGGGGAGCAGUUUUAGAGCUAGAA | 1284 |
| _20nt_3- | AUAGCAAGUUAAAAUAAGGCUAGUCCGUUAUCAACUUGAAA | |
| 9_002_+_31946073_spCas9 | AAGUGGCACCGAGUCGGUGCmUsmUsmUsU | |
| GuideDesign90_ABE_NGG | mCsmUsmGsGGGGCUGGUGGGGAGCAGUUUUAGAGCUAGAA | 1285 |
| _20nt_3- | AUAGCAAGUUAAAAUAAGGCUAGUCCGUUAUCAACUUGAAA | |
| 12_018_+_31946073_spCas9 | AAGUGGCACCGAGUCGGUGCmUsmUsmUsU | |
| GuideDesign90_CBE_NGG | mCsmUsmGsGGGGCUGGUGGGGAGCAGUUUUAGAGCUAGAA | 1286 |
| _20nt_4- | AUAGCAAGUUAAAAUAAGGCUAGUCCGUUAUCAACUUGAAA | |
| 9_003_+_31946073_spCas9 | AAGUGGCACCGAGUCGGUGCmUsmUsmUsU | |
| GuideDesign90_CBE_NGG | mCsmUsmGsGGGGCUGGUGGGGAGCAGUUUUAGAGCUAGAA | 1287 |
| _20nt_3- | AUAGCAAGUUAAAAUAAGGCUAGUCCGUUAUCAACUUGAAA | |
| 9_029_+_31946073_spCas9 | AAGUGGCACCGAGUCGGUGCmUsmUsmUsU | |
| GuideDesign90_CBE_NGG | mCsmUsmGsGGGGCUGGUGGGGAGCAGUUUUAGAGCUAGAA | 1288 |
| _20nt_3- | AUAGCAAGUUAAAAUAAGGCUAGUCCGUUAUCAACUUGAAA | |
| 16_033_+_31946073_SpCas | AAGUGGCACCGAGUCGGUGCmUsmUsmUsU | |
| 9 | | |
| GuideDesign90_ABE_NG | mCsmUsmGsGGGGCUGGUGGGGAGCAGUUUUAGAGCUAGAA | 1289 |
| _20nt_3- | AUAGCAAGUUAAAAUAAGGCUAGUCCGUUAUCAACUUGAAA | |
| 9_027_+_31946073_spCas9 | AAGUGGCACCGAGUCGGUGCmUsmUsmUsU | |
| GuideDesign90_CBE_NG | mCsmUsmGsGGGGCUGGUGGGGAGCAGUUUUAGAGCUAGAA | 1290 |
| _20nt_4- | AUAGCAAGUUAAAAUAAGGCUAGUCCGUUAUCAACUUGAAA | |
| 9_028_+_31946073_spCas9 | AAGUGGCACCGAGUCGGUGCmUsmUsmUsU | |
| GuideDesign90_ABE_NRN | mCsmUsmGsGGGGCUGGUGGGGAGCAGUUUUAGAGCUAGAA | 1291 |
| _20nt_3- | AUAGCAAGUUAAAAUAAGGCUAGUCCGUUAUCAACUUGAAA | |
| 9_021_+_31946073_spCas9 | AAGUGGCACCGAGUCGGUGCmUsmUsmUsU | |
| GuideDesign90_CBE_NRN | mCsmUsmGsGGGGCUGGUGGGGAGCAGUUUUAGAGCUAGAA | 1292 |
| _20nt_3- | AUAGCAAGUUAAAAUAAGGCUAGUCCGUUAUCAACUUGAAA | |
| 9_022_+_31946073_spCas9 | AAGUGGCACCGAGUCGGUGCmUsmUsmUsU | |
| GuideDesign90_MBE_NRN | mCsmUsmGsGGGGCUGGUGGGGAGCAGUUUUAGAGCUAGAA | 1293 |
| _20nt_3- | AUAGCAAGUUAAAAUAAGGCUAGUCCGUUAUCAACUUGAAA | |
| 9_023_+_31946073_spCas9 | AAGUGGCACCGAGUCGGUGCmUsmUsmUsU | |
| GuideDesign90_ABE_NGG | mCsmUsmUsUUAUACAAUCUGUGUUCGUUUUAGAGCUAGAA | 1294 |
| _20nt_3-12_018_−_ | AUAGCAAGUUAAAAUAAGGCUAGUCCGUUAUCAACUUGAAA | |
| 31946049_spCas9 | AAGUGGCACCGAGUCGGUGCmUsmUsmUsU | |
| GuideDesign90_ABE_NGG | mCsmUsmUsUUAUACAAUCUGUGUUCGUUUUAGAGCUAGAA | 1295 |
| _20nt_13-16_032_−_ | AUAGCAAGUUAAAAUAAGGCUAGUCCGUUAUCAACUUGAAA | |
| 31946049_SpCas9 | AAGUGGCACCGAGUCGGUGCmUsmUsmUsU | |
| GuideDesign90_CBE_NGG | mCsmUsmUsUUAUACAAUCUGUGUUCGUUUUAGAGCUAGAA | 1296 |
| _20nt_4-9_003_−_ | AUAGCAAGUUAAAAUAAGGCUAGUCCGUUAUCAACUUGAAA | |
| 31946049_spCas9 | AAGUGGCACCGAGUCGGUGCmUsmUsmUsU | |
| GuideDesign90_CBE_NGG | mCsmUsmUsUUAUACAAUCUGUGUUCGUUUUAGAGCUAGAA | 1297 |
| _20nt_3-9_029_−_ | AUAGCAAGUUAAAAUAAGGCUAGUCCGUUAUCAACUUGAAA | |
| 31946049_spCas9 | AAGUGGCACCGAGUCGGUGCmUsmUsmUsU | |
| GuideDesign90_CBE_NGG | mCsmUsmUsUUAUACAAUCUGUGUUCGUUUUAGAGCUAGAA | 1298 |
| _20nt_3-16_033_−_ | AUAGCAAGUUAAAAUAAGGCUAGUCCGUUAUCAACUUGAAA | |
| 31946049_SpCas9 | AAGUGGCACCGAGUCGGUGCmUsmUsmUsU | |
| GuideDesign90_ABE_NG | mCsmUsmUsUUAUACAAUCUGUGUUCGUUUUAGAGCUAGAA | 1299 |
| _20nt_3-9_027_−_ | AUAGCAAGUUAAAAUAAGGCUAGUCCGUUAUCAACUUGAAA | |
| 31946050_spCas9 | AAGUGGCACCGAGUCGGUGCmUsmUsmUsU | |
| GuideDesign90_CBE_NG | mCsmUsmUsUUAUACAAUCUGUGUUCGUUUUAGAGCUAGAA | 1300 |
| _20nt_4-9_028_−_ | AUAGCAAGUUAAAAUAAGGCUAGUCCGUUAUCAACUUGAAA | |
| 31946050_spCas9 | AAGUGGCACCGAGUCGGUGCmUsmUsmUsU | |
| GuideDesign90_ABE_NRN | mCsmUsmUsUUAUACAAUCUGUGUUCGUUUUAGAGCUAGAA | 1301 |
| _20nt_3-9_021_−_ | AUAGCAAGUUAAAAUAAGGCUAGUCCGUUAUCAACUUGAAA | |
| 31946049_spCas9 | AAGUGGCACCGAGUCGGUGCmUsmUsmUsU | |
| GuideDesign90_CBE_NRN | mCsmUsmUsUUAUACAAUCUGUGUUCGUUUUAGAGCUAGAA | 1302 |
| _20nt_3-9_022_−_ | AUAGCAAGUUAAAAUAAGGCUAGUCCGUUAUCAACUUGAAA | |
| 31946049_spCas9 | AAGUGGCACCGAGUCGGUGCmUsmUsmUSU | |
| Guide_Design90_MBE_NRN | mCsmUsmUsUUAUACAAUCUGUGUUCGUUUUAGAGCUAGAA | 1303 |
| _20nt_3-9_023_−_ | AUAGCAAGUUAAAAUAAGGCUAGUCCGUUAUCAACUUGAAA | |
| 31946049_spCas9 | AAGUGGCACCGAGUCGGUGCmUsmUsmUsU | |
| GuideDesign90_CBE_NRRH | mCsmUsmUsUUAUACAAUCUGUGUUCGUUUUAGAGCUAGAA | 1304 |
| _20nt_3-9_031_−_ | AUAGCAAGUUAAAAUAAGGCUAGUCCGUUAUCAACUUGAAA | |
| 31946048_spCas9 | AAGUGGCACCGAGUCGGUGCmUsmUsmUsU | |
| GuideDesign90_ABE_NGG | mGsmCsmUsGGGGGCUGGUGGGGAGCGUUUUAGAGCUAGAA | 1305 |
| _20nt_3- | AUAGCAAGUUAAAAUAAGGCUAGUCCGUUAUCAACUUGAAA | |
| 9_002_+_31946072_spCas9 | AAGUGGCACCGAGUCGGUGCmUsmUsmUsU | |
| GuideDesign90_ABE_NGG | mGsmCsmUsGGGGGCUGGUGGGGAGCGUUUUAGAGCUAGAA | 1306 |
| _20nt_3- | AUAGCAAGUUAAAAUAAGGCUAGUCCGUUAUCAACUUGAAA | |
| 12_018_+_31946072_spCas9 | AAGUGGCACCGAGUCGGUGCmUsmUsmUsU | |
| GuideDesign90_CBE_NGG | mGsmCsmUsGGGGGCUGGUGGGGAGCGUUUUAGAGCUAGAA | 1307 |
| _20nt_4- | AUAGCAAGUUAAAAUAAGGCUAGUCCGUUAUCAACUUGAAA | |
| 9_003_+_31946072_spCas9 | AAGUGGCACCGAGUCGGUGCmUsmUsmUsU | |
| GuideDesign90_CBE_NGG | mGsmCsmUsGGGGGCUGGUGGGGAGCGUUUUAGAGCUAGAA | 1308 |
| _20nt_3- | AUAGCAAGUUAAAAUAAGGCUAGUCCGUUAUCAACUUGAAA | |
| 9_029_+_31946072_spCas9 | AAGUGGCACCGAGUCGGUGCmUsmUsmUsU | |
| GuideDesign90_CBE_NGG | mGsmCsmUsGGGGGCUGGUGGGGAGCGUUUUAGAGCUAGAA | 1309 |
| _20nt_3- | AUAGCAAGUUAAAAUAAGGCUAGUCCGUUAUCAACUUGAAA | |
| 16_033_+_31946072_SpCas9 | AAGUGGCACCGAGUCGGUGCmUsmUsmUSU | |
| GuideDesign90_ABE_NG | mGsmCsmUsGGGGGCUGGUGGGGAGCGUUUUAGAGCUAGAA | 1310 |
| _20nt_3- | AUAGCAAGUUAAAAUAAGGCUAGUCCGUUAUCAACUUGAAA | |
| 9_027_+_31946072_spCas9 | AAGUGGCACCGAGUCGGUGCmUsmUsmUsU | |
| GuideDesign90_CBE_NG | mGsmCsmUsGGGGGCUGGUGGGGAGCGUUUUAGAGCUAGAA | 1311 |
| _20nt_4- | AUAGCAAGUUAAAAUAAGGCUAGUCCGUUAUCAACUUGAAA | |
| 9_028_+_31946072_spCas9 | AAGUGGCACCGAGUCGGUGCmUsmUsmUsU | |
| GuideDesign90_ABE_NRN | mGsmCsmUsGGGGGCUGGUGGGGAGCGUUUUAGAGCUAGAA | 1312 |
| _20nt_3- | AUAGCAAGUUAAAAUAAGGCUAGUCCGUUAUCAACUUGAAA | |
| 9_021_+_31946072_spCas9 | AAGUGGCACCGAGUCGGUGCmUsmUsmUsU | |
| GuideDesign90_CBE_NRN | mGsmCsmUsGGGGGCUGGUGGGGAGCGUUUUAGAGCUAGAA | 1313 |
| _20nt_3- | AUAGCAAGUUAAAAUAAGGCUAGUCCGUUAUCAACUUGAAA | |
| 9_022_+_31946072_spCas9 | AAGUGGCACCGAGUCGGUGCmUsmUsmUSU | |
| Guide_Design90_MBE_NRN | mGsmCsmUsGGGGGCUGGUGGGGAGCGUUUUAGAGCUAGAA | 1314 |
| _20nt_3- | AUAGCAAGUUAAAAUAAGGCUAGUCCGUUAUCAACUUGAAA | |
| 9_023_+_31946072_spCas9 | AAGUGGCACCGAGUCGGUGCmUsmUsmUsU | |
| Guide_Design90_ABE_NRN | mGsmGsmCsUGGGGGCUGGUGGGGAGGUUUUAGAGCUAGAA | 1315 |
| _20nt_3- | AUAGCAAGUUAAAAUAAGGCUAGUCCGUUAUCAACUUGAAA | |
| 9_021_+_31946071_spCas9 | AAGUGGCACCGAGUCGGUGCmUsmUsmUsU | |
| GuideDesign90_CBE_NRN | mGsmGsmCSUGGGGGCUGGUGGGGAGGUUUUAGAGCUAGAA | 1316 |
| _20nt_3- | AUAGCAAGUUAAAAUAAGGCUAGUCCGUUAUCAACUUGAAA | |
| 9_022_+_31946071_spCas9 | AAGUGGCACCGAGUCGGUGCmUsmUsmUSU | |
| GuideDesign90_MBE_NRN | mGsmGsmCsUGGGGGCUGGUGGGGAGGUUUUAGAGCUAGAA | 1317 |
| _20nt_3- | AUAGCAAGUUAAAAUAAGGCUAGUCCGUUAUCAACUUGAAA | |
| 9_023_+_31946071_spCas9 | AAGUGGCACCGAGUCGGUGCmUsmUsmUsU | |
| GuideDesign90_ABE_NGG | mUsmAsmAsAAGGCUGGGGGCUGGUGGUUUUAGAGCUAGAA | 1318 |
| _20nt_3- | AUAGCAAGUUAAAAUAAGGCUAGUCCGUUAUCAACUUGAAA | |
| 12_018_+_31946066_spCas9 | AAGUGGCACCGAGUCGGUGCmUsmUsmUsU | |
| GuideDesign90_CBE_NGG | mUsmAsmAsAAGGCUGGGGGCUGGUGGUUUUAGAGCUAGAA | 1319 |
| _20nt_4- | AUAGCAAGUUAAAAUAAGGCUAGUCCGUUAUCAACUUGAAA | |
| 9_003_+_31946066_spCas9 | AAGUGGCACCGAGUCGGUGCmUsmUsmUSU | |
| GuideDesign90_CBE_NGG | mUsmAsmAsAAGGCUGGGGGCUGGUGGUUUUAGAGCUAGAA | 1320 |
| _20nt_3- | AUAGCAAGUUAAAAUAAGGCUAGUCCGUUAUCAACUUGAAA | |
| 9_029_+_31946066_spCas9 | AAGUGGCACCGAGUCGGUGCmUsmUsmUsU | |
| GuideDesign90_CBE_NGG | mUsmAsmAsAAGGCUGGGGGCUGGUGGUUUUAGAGCUAGAA | 1321 |
| _20nt_3- | AUAGCAAGUUAAAAUAAGGCUAGUCCGUUAUCAACUUGAAA | |
| 16_033_+_31946066_SpCas | AAGUGGCACCGAGUCGGUGCmUsmUsmUsU | |
| 9 | | |
| GuideDesign90_ABE_NG | mUsmAsmAsAAGGCUGGGGGCUGGUGGUUUUAGAGCUAGAA | 1322 |
| _20nt_3- | AUAGCAAGUUAAAAUAAGGCUAGUCCGUUAUCAACUUGAAA | |
| 9_027_+_31946066_spCas9 | AAGUGGCACCGAGUCGGUGCmUsmUsmUsU | |
| GuideDesign90_CBE_NG | mUsmAsmAsAAGGCUGGGGGCUGGUGGUUUUAGAGCUAGAA | 1323 |
| _20nt_4- | AUAGCAAGUUAAAAUAAGGCUAGUCCGUUAUCAACUUGAAA | |
| 9_028_+_31946066_spCas9 | AAGUGGCACCGAGUCGGUGCmUsmUsmUsU | |
| GuideDesign90_ABE_NRN | mUsmAsmAsAAGGCUGGGGGCUGGUGGUUUUAGAGCUAGAA | 1324 |
| _20nt_3- | AUAGCAAGUUAAAAUAAGGCUAGUCCGUUAUCAACUUGAAA | |
| 9_021_+_31946066_spCas9 | AAGUGGCACCGAGUCGGUGCmUsmUsmUsU | |
| GuideDesign90_CBE_NRN | mUsmAsmAsAAGGCUGGGGGCUGGUGGUUUUAGAGCUAGAA | 1325 |
| _20nt_3- | AUAGCAAGUUAAAAUAAGGCUAGUCCGUUAUCAACUUGAAA | |
| 9_022_+_31946066_spCas9 | AAGUGGCACCGAGUCGGUGCmUsmUsmUsU | |
| GuideDesign90_MBE_NRN | mUsmAsmAsAAGGCUGGGGGCUGGUGGUUUUAGAGCUAGAA | 1326 |
| _20nt_3- | AUAGCAAGUUAAAAUAAGGCUAGUCCGUUAUCAACUUGAAA | |
| 9_023_+_31946066_spCas9 | AAGUGGCACCGAGUCGGUGCmUsmUsmUsU | |
| GuideDesign90_CBE_NRRH | mUsmAsmAsAAGGCUGGGGGCUGGUGGUUUUAGAGCUAGAA | 1327 |
| _20nt_3- | AUAGCAAGUUAAAAUAAGGCUAGUCCGUUAUCAACUUGAAA | |
| 9_031_+_31946066_spCas9 | AAGUGGCACCGAGUCGGUGCmUsmUsmUsU | |
| GuideDesign90_ABE_NGG | mUsmAsmUsAAAAGGCUGGGGGCUGGGUUUUAGAGCUAGAA | 1328 |
| _20nt_3- | AUAGCAAGUUAAAAUAAGGCUAGUCCGUUAUCAACUUGAAA | |
| 12_018_+_31946064_spCas9 | AAGUGGCACCGAGUCGGUGCmUsmUsmUsU | |
| GuideDesign90_ABE_NGG | mUsmAsmUsAAAAGGCUGGGGGCUGGGUUUUAGAGCUAGAA | 1329 |
| _20nt_13- | AUAGCAAGUUAAAAUAAGGCUAGUCCGUUAUCAACUUGAAA | |
| 16_032_+_31946064_SpCas | AAGUGGCACCGAGUCGGUGCmUsmUsmUSU | |
| 9 | | |
| GuideDesign90_CBE_NGG | mUsmAsmUsAAAAGGCUGGGGGCUGGGUUUUAGAGCUAGAA | 1330 |
| _20nt_4- | AUAGCAAGUUAAAAUAAGGCUAGUCCGUUAUCAACUUGAAA | |
| 9_003_+_31946064_spCas9 | AAGUGGCACCGAGUCGGUGCmUsmUsmUsU | |
| GuideDesign90_CBE_NGG | mUsmAsmUsAAAAGGCUGGGGGCUGGGUUUUAGAGCUAGAA | 1331 |
| _20nt_3- | AUAGCAAGUUAAAAUAAGGCUAGUCCGUUAUCAACUUGAAA | |
| 9_029_+_31946064_spCas9 | AAGUGGCACCGAGUCGGUGCmUsmUsmUsU | |
| GuideDesign90_CBE_NGG | mUsmAsmUsAAAAGGCUGGGGGCUGGGUUUUAGAGCUAGAA | 1332 |
| _20nt_3- | AUAGCAAGUUAAAAUAAGGCUAGUCCGUUAUCAACUUGAAA | |
| 16_033_+_31946064_SpCas | AAGUGGCACCGAGUCGGUGCmUsmUsmUsU | |
| 9 | | |
| GuideDesign90_ABE_NG | mUsmAsmUsAAAAGGCUGGGGGCUGGGUUUUAGAGCUAGAA | 1333 |
| _20nt_3- | AUAGCAAGUUAAAAUAAGGCUAGUCCGUUAUCAACUUGAAA | |
| 9_027_+_31946064_spCas9 | AAGUGGCACCGAGUCGGUGCmUsmUsmUsU | |
| GuideDesign90_CBE_NG | mUsmAsmUsAAAAGGCUGGGGGCUGGGUUUUAGAGCUAGAA | 1334 |
| _20nt_4- | AUAGCAAGUUAAAAUAAGGCUAGUCCGUUAUCAACUUGAAA | |
| 9_028_+_31946064_spCas9 | AAGUGGCACCGAGUCGGUGCmUsmUsmUsU | |
| GuideDesign90_ABE_NRN | mUsmAsmUsAAAAGGCUGGGGGCUGGGUUUUAGAGCUAGAA | 1335 |
| _20nt_3- | AUAGCAAGUUAAAAUAAGGCUAGUCCGUUAUCAACUUGAAA | |
| 9_021_+_31946064_spCas9 | AAGUGGCACCGAGUCGGUGCmUsmUsmUsU | |
| GuideDesign90_CBE_NRN | mUsmAsmUsAAAAGGCUGGGGGCUGGGUUUUAGAGCUAGAA | 1336 |
| _20nt_3- | AUAGCAAGUUAAAAUAAGGCUAGUCCGUUAUCAACUUGAAA | |
| 9_022_+_31946064_spCas9 | AAGUGGCACCGAGUCGGUGCmUsmUsmUsU | |
| GuideDesign90_MBE_NRN | mUsmAsmUsAAAAGGCUGGGGGCUGGGUUUUAGAGCUAGAA | 1337 |
| _20nt_3- | AUAGCAAGUUAAAAUAAGGCUAGUCCGUUAUCAACUUGAAA | |
| 9_023_+_31946064_spCas9 | AAGUGGCACCGAGUCGGUGCmUsmUsmUsU | |
| GuideDesign90_ABE_VTTN | mGsmUsmUsCUGUCUUUUGGUCAGGACAACCGUCUAGCUAU | 1338 |
| 22nt_5- | AAGUGCUGCAGGGUGUGAGAAACUCCUAUUGCUGGACGAUG | |
| 9_017_+_31946060_cas12b | UCUCUUACGAGGCAUUAGCACUAUAAAAGGCUGGGGGCUGS | |
| | mGsmUsmG | |
| GuideDesign90_ABE_VTTN | mGsmUsmUsCUGUCUUUUGGUCAGGACAACCGUCUAGCUAU | 1339 |
| 22nt_5-9_017_−_ | AAGUGCUGCAGGGUGUGAGAAACUCCUAUUGCUGGACGAUG | |
| 31946046_cas12b | UCUCUUACGAGGCAUUAGCACUAUACAAUCUGUGUUCUGGS | |
| | mCsmAsmC | |
| GuideDesign90_ABE_NGG | mUsmGsmGsGGGCUGGUGGGGAGCAGGUUUUAGAGCUAGAA | 1340 |
| _20nt_3- | AUAGCAAGUUAAAAUAAGGCUAGUCCGUUAUCAACUUGAAA | |
| 9_002_+_31946074_spCas9 | AAGUGGCACCGAGUCGGUGCmUsmUsmUsU | |
| GuideDesign90_ABE_NGG | mUsmGsmGsGGGCUGGUGGGGAGCAGGUUUUAGAGCUAGAA | 1341 |
| _20nt_3- | AUAGCAAGUUAAAAUAAGGCUAGUCCGUUAUCAACUUGAAA | |
| 12_018_+_31946074_spCas9 | AAGUGGCACCGAGUCGGUGCmUsmUsmUsU | |
| GuideDesign90_CBE_NGG | mUsmGsmGsGGGCUGGUGGGGAGCAGGUUUUAGAGCUAGAA | 1342 |
| _20nt_3- | AUAGCAAGUUAAAAUAAGGCUAGUCCGUUAUCAACUUGAAA | |
| 9_029_+_31946074_spCas9 | AAGUGGCACCGAGUCGGUGCmUsmUsmUsU | |
| GuideDesign90_CBE_NGG | mUsmGsmGsGGGCUGGUGGGGAGCAGGUUUUAGAGCUAGAA | 1343 |
| _20nt_3- | AUAGCAAGUUAAAAUAAGGCUAGUCCGUUAUCAACUUGAAA | |
| 16_033_+_31946074_SpCas | AAGUGGCACCGAGUCGGUGCmUsmUsmUsU | |
| 9 | | |
| GuideDesign90_ABE_NG | mUsmGsmGsGGGCUGGUGGGGAGCAGGUUUUAGAGCUAGAA | 1344 |
| _20nt_3- | AUAGCAAGUUAAAAUAAGGCUAGUCCGUUAUCAACUUGAAA | |
| 9_027_+_31946074_spCas9 | AAGUGGCACCGAGUCGGUGCmUsmUsmUSU | |
| GuideDesign90_ABE_NRN | mUsmGsmGsGGGCUGGUGGGGAGCAGGUUUUAGAGCUAGAA | 1345 |
| _20nt_3- | AUAGCAAGUUAAAAUAAGGCUAGUCCGUUAUCAACUUGAAA | |
| 9_021_+_31946074_spCas9 | AAGUGGCACCGAGUCGGUGCmUsmUsmUsU | |
| GuideDesign90_CBE_NRN | mUsmGsmGsGGGCUGGUGGGGAGCAGGUUUUAGAGCUAGAA | 1346 |
| _20nt_3- | AUAGCAAGUUAAAAUAAGGCUAGUCCGUUAUCAACUUGAAA | |
| 9_022_+_31946074_spCas9 | AAGUGGCACCGAGUCGGUGCmUsmUsmUsU | |
| GuideDesign90_MBE_NRN | mUsmGsmGsGGGCUGGUGGGGAGCAGGUUUUAGAGCUAGAA | 1347 |
| _20nt_3- | AUAGCAAGUUAAAAUAAGGCUAGUCCGUUAUCAACUUGAAA | |
| 9_023_+_31946074_spCas9 | AAGUGGCACCGAGUCGGUGCmUsmUsmUSU | |
| GuideDesign90_ABE_NG | mUsmGsmUsAUAAAAGGCUGGGGGCUGUUUUAGAGCUAGAA | 1348 |
| _20nt_3- | AUAGCAAGUUAAAAUAAGGCUAGUCCGUUAUCAACUUGAAA | |
| 9_027_+_31946062_spCas9 | AAGUGGCACCGAGUCGGUGCmUsmUsmUsU | |
| GuideDesign90_CBE_NG | mUsmGsmUsAUAAAAGGCUGGGGGCUGUUUUAGAGCUAGAA | 1349 |
| _20nt_4- | AUAGCAAGUUAAAAUAAGGCUAGUCCGUUAUCAACUUGAAA | |
| 9_028_+_31946062_spCas9 | AAGUGGCACCGAGUCGGUGCmUsmUsmUsU | |
| GuideDesign90_ABE_NRN | mUsmGsmUsAUAAAAGGCUGGGGGCUGUUUUAGAGCUAGAA | 1350 |
| _20nt_3- | AUAGCAAGUUAAAAUAAGGCUAGUCCGUUAUCAACUUGAAA | |
| 9_021_+_31946062_spCas9 | AAGUGGCACCGAGUCGGUGCmUsmUsmUsU | |
| GuideDesign90_CBE_NRN | mUsmGsmUsAUAAAAGGCUGGGGGCUGUUUUAGAGCUAGAA | 1351 |
| _20nt_3- | AUAGCAAGUUAAAAUAAGGCUAGUCCGUUAUCAACUUGAAA | |
| 9_022_+_31946062_spCas9 | AAGUGGCACCGAGUCGGUGCmUsmUsmUsU | |
| GuideDesign90_MBE_NRN | mUsmGsmUsAUAAAAGGCUGGGGGCUGUUUUAGAGCUAGAA | 1352 |
| _20nt_3- | AUAGCAAGUUAAAAUAAGGCUAGUCCGUUAUCAACUUGAAA | |
| 9_023_+_31946062_spCas9 | AAGUGGCACCGAGUCGGUGCmUsmUsmUsU | |
| GuideDesign90_ABE_NRN | mUsmUsmASUACAAUCUGUGUUCUGGGUUUUAGAGCUAGAA | 1353 |
| _20nt_3-9_021_−_ | AUAGCAAGUUAAAAUAAGGCUAGUCCGUUAUCAACUUGAAA | |
| 31946046_spCas9 | AAGUGGCACCGAGUCGGUGCmUsmUsmUsU | |
| GuideDesign90_CBE_NRN | mUsmUsmASUACAAUCUGUGUUCUGGGUUUUAGAGCUAGAA | 1354 |
| _20nt_3-9_022_−_ | AUAGCAAGUUAAAAUAAGGCUAGUCCGUUAUCAACUUGAAA | |
| 31946046_spCas9 | AAGUGGCACCGAGUCGGUGCmUsmUsmUsU | |
| Guide_Design90_MBE_NRN | mUsmUsmASUACAAUCUGUGUUCUGGGUUUUAGAGCUAGAA | 1355 |
| _20nt_3-9_023_−_ | AUAGCAAGUUAAAAUAAGGCUAGUCCGUUAUCAACUUGAAA | |
| 31946046_spCas9 | AAGUGGCACCGAGUCGGUGCmUsmUsmUSU | |
| GuideDesign90_ABE_NRCH | mUsmUsmASUACAAUCUGUGUUCUGGGUUUUAGAGCUAGAA | 1356 |
| _20nt_3-9_024_−_ | AUAGCAAGUUAAAAUAAGGCUAGUCCGUUAUCAACUUGAAA | |
| 31946045_spCas9 | AAGUGGCACCGAGUCGGUGCmUsmUsmUsU | |
| GuideDesign90_CBE_NRCH | mUsmUsmASUACAAUCUGUGUUCUGGGUUUUAGAGCUAGAA | 1357 |
| _20nt_3-9_030_−_ | AUAGCAAGUUAAAAUAAGGCUAGUCCGUUAUCAACUUGAAA | |
| 31946045_spCas9 | AAGUGGCACCGAGUCGGUGCmUsmUsmUsU | |
| GuideDesign90_ABE_NGG | mUsmUsmGsUAUAAAAGGCUGGGGGCGUUUUAGAGCUAGAA | 1358 |
| _20nt_3- | AUAGCAAGUUAAAAUAAGGCUAGUCCGUUAUCAACUUGAAA | |
| 9_002_+_31946061_spCas9 | AAGUGGCACCGAGUCGGUGCmUsmUsmUsU | |
| GuideDesign90_ABE_NGG | mUsmUsmGsUAUAAAAGGCUGGGGGCGUUUUAGAGCUAGAA | 1359 |
| _20nt_3- | AUAGCAAGUUAAAAUAAGGCUAGUCCGUUAUCAACUUGAAA | |
| 12_018_+_31946061_spCas9 | AAGUGGCACCGAGUCGGUGCmUsmUsmUsU | |
| GuideDesign90_ABE_NGG | mUsmUsmGsUAUAAAAGGCUGGGGGCGUUUUAGAGCUAGAA | 1360 |
| _20nt_13- | AUAGCAAGUUAAAAUAAGGCUAGUCCGUUAUCAACUUGAAA | |
| 16_032_+_31946061_SpCas | AAGUGGCACCGAGUCGGUGCmUsmUsmUSU | |
| 9 | | |
| GuideDesign90_CBE_NGG | mUsmUsmGsUAUAAAAGGCUGGGGGCGUUUUAGAGCUAGAA | 1361 |
| _20nt_4- | AUAGCAAGUUAAAAUAAGGCUAGUCCGUUAUCAACUUGAAA | |
| 9_003_+_31946061_spCas9 | AAGUGGCACCGAGUCGGUGCmUsmUsmUSU | |
| GuideDesign90_CBE_NGG | mUsmUsmGsUAUAAAAGGCUGGGGGCGUUUUAGAGCUAGAA | 1362 |
| _20nt_3- | AUAGCAAGUUAAAAUAAGGCUAGUCCGUUAUCAACUUGAAA | |
| 9_029_+_31946061_spCas9 | AAGUGGCACCGAGUCGGUGCmUsmUsmUsU | |
| GuideDesign90_CBE_NGG | mUsmUsmGsUAUAAAAGGCUGGGGGCGUUUUAGAGCUAGAA | 1363 |
| _20nt_3- | AUAGCAAGUUAAAAUAAGGCUAGUCCGUUAUCAACUUGAAA | |
| 16_033_+_31946061_SpCas9 | AAGUGGCACCGAGUCGGUGCmUsmUsmUsU | |
| GuideDesign90_ABE_NG | mUsmUsmGsUAUAAAAGGCUGGGGGCGUUUUAGAGCUAGAA | 1364 |
| _20nt_3- | AUAGCAAGUUAAAAUAAGGCUAGUCCGUUAUCAACUUGAAA | |
| 9_027_+_31946061_spCas9 | AAGUGGCACCGAGUCGGUGCmUsmUsmUsU | |
| GuideDesign90_CBE_NG | mUsmUsmGsUAUAAAAGGCUGGGGGCGUUUUAGAGCUAGAA | 1365 |
| _20nt_4- | AUAGCAAGUUAAAAUAAGGCUAGUCCGUUAUCAACUUGAAA | |
| 9_028_+_31946061_spCas9 | AAGUGGCACCGAGUCGGUGCmUsmUsmUSU | |
| GuideDesign90_ABE_NRN | mUsmUsmGsUAUAAAAGGCUGGGGGCGUUUUAGAGCUAGAA | 1366 |
| _20nt_3- | AUAGCAAGUUAAAAUAAGGCUAGUCCGUUAUCAACUUGAAA | |
| 9_021_+_31946061_spCas9 | AAGUGGCACCGAGUCGGUGCmUsmUsmUsU | |
| GuideDesign90_CBE_NRN | mUsmUsmGsUAUAAAAGGCUGGGGGCGUUUUAGAGCUAGAA | 1367 |
| _20nt_3- | AUAGCAAGUUAAAAUAAGGCUAGUCCGUUAUCAACUUGAAA | |
| 9_022_+_31946061_spCas9 | AAGUGGCACCGAGUCGGUGCmUsmUsmUsU | |
| GuideDesign90_MBE_NRN | mUsmUsmGsUAUAAAAGGCUGGGGGCGUUUUAGAGCUAGAA | 1368 |
| _20nt_3- | AUAGCAAGUUAAAAUAAGGCUAGUCCGUUAUCAACUUGAAA | |
| 9_023_+_31946061_spCas9 | AAGUGGCACCGAGUCGGUGCmUsmUsmUsU | |
| Guide_Design90_CBE_NRRH | mUsmUsmGsUAUAAAAGGCUGGGGGCGUUUUAGAGCUAGAA | 1369 |
| _20nt_3- | AUAGCAAGUUAAAAUAAGGCUAGUCCGUUAUCAACUUGAAA | |
| 9_031_+_31946061_spCas9 | AAGUGGCACCGAGUCGGUGCmUsmUsmUsU | |
| GuideDesign90_ABE_NGC | mUsmUsmUSUAUACAAUCUGUGUUCUGUUUUAGAGCUAGAA | 1370 |
| _20nt_3-9_008_−_ | AUAGCAAGUUAAAAUAAGGCUAGUCCGUUAUCAACUUGAAA | |
| 31946048_spCas9 | AAGUGGCACCGAGUCGGUGCmUsmUsmUSU | |
| GuideDesign90_ABE_NGC | mUsmUsmUSUAUACAAUCUGUGUUCUGUUUUAGAGCUAGAA | 1371 |
| _20nt_3-12_020_−_ | AUAGCAAGUUAAAAUAAGGCUAGUCCGUUAUCAACUUGAAA | |
| 31946048_spCas9 | AAGUGGCACCGAGUCGGUGCmUsmUsmUSU | |
| GuideDesign90_CBE_NGC | mUsmUsmUSUAUACAAUCUGUGUUCUGUUUUAGAGCUAGAA | 1372 |
| _20nt_4-9_009_−_ | AUAGCAAGUUAAAAUAAGGCUAGUCCGUUAUCAACUUGAAA | |
| 31946048_spCas9 | AAGUGGCACCGAGUCGGUGCmUsmUsmUsU | |
| GuideDesign90_ABE_NG | mUsmUsmUsUAUACAAUCUGUGUUCUGUUUUAGAGCUAGAA | 1373 |
| _20nt_3-9_027_−_ | AUAGCAAGUUAAAAUAAGGCUAGUCCGUUAUCAACUUGAAA | |
| 31946049_spCas9 | AAGUGGCACCGAGUCGGUGCmUsmUsmUsU | |
| GuideDesign90_CBE_NG | mUsmUsmUsUAUACAAUCUGUGUUCUGUUUUAGAGCUAGAA | 1374 |
| _20nt_4-9_028_−_ | AUAGCAAGUUAAAAUAAGGCUAGUCCGUUAUCAACUUGAAA | |
| 31946049_spCas9 | AAGUGGCACCGAGUCGGUGCmUsmUsmUsU | |
| GuideDesign90_ABE_NRN | mUsmUsmUsUAUACAAUCUGUGUUCUGUUUUAGAGCUAGAA | 1375 |
| _20nt_3-9_021_−_ | AUAGCAAGUUAAAAUAAGGCUAGUCCGUUAUCAACUUGAAA | |
| 31946048_spCas9 | AAGUGGCACCGAGUCGGUGCmUsmUsmUsU | |
| GuideDesign90_CBE_NRN | mUsmUsmUSUAUACAAUCUGUGUUCUGUUUUAGAGCUAGAA | 1376 |
| _20nt_3-9_022_−_ | AUAGCAAGUUAAAAUAAGGCUAGUCCGUUAUCAACUUGAAA | |
| 31946048_spCas9 | AAGUGGCACCGAGUCGGUGCmUsmUsmUsU | |
| GuideDesign90_MBE_NRN | mUsmUsmUsUAUACAAUCUGUGUUCUGUUUUAGAGCUAGAA | 1377 |
| _20nt_3-9_023_−_ | AUAGCAAGUUAAAAUAAGGCUAGUCCGUUAUCAACUUGAAA | |
| 31946048_spCas9 | AAGUGGCACCGAGUCGGUGCmUsmUsmUsU | |
| GuideDesign90_ABE_NRCH | mUsmUsmUSUAUACAAUCUGUGUUCUGUUUUAGAGCUAGAA | 1378 |
| _20nt_3-9_024_−_ | AUAGCAAGUUAAAAUAAGGCUAGUCCGUUAUCAACUUGAAA | |
| 31946047_spCas9 | AAGUGGCACCGAGUCGGUGCmUsmUsmUSU | |
| GuideDesign90_CBE_NRCH | mUsmUsmUSUAUACAAUCUGUGUUCUGUUUUAGAGCUAGAA | 1379 |
| _20nt_3-9_030_−_ | AUAGCAAGUUAAAAUAAGGCUAGUCCGUUAUCAACUUGAAA | |
| 31946047_spCas9 | AAGUGGCACCGAGUCGGUGCmUsmUsmUsU | |
| GuideDesign90_ABE_NRN | mAsmAsmCsGCCAUGGGGAGCAAUCUGUUUUAGAGCUAGAA | 1380 |
| _20nt_3- | AUAGCAAGUUAAAAUAAGGCUAGUCCGUUAUCAACUUGAAA | |
| 9_021_+_31946215_spCas9 | AAGUGGCACCGAGUCGGUGCmUsmUsmUSU | |
| GuideDesign90_MBE_NRN | mAsmAsmCsGCCAUGGGGAGCAAUCUGUUUUAGAGCUAGAA | 1381 |
| _20nt_3- | AUAGCAAGUUAAAAUAAGGCUAGUCCGUUAUCAACUUGAAA | |
| 9_023_+_31946215_spCas9 | AAGUGGCACCGAGUCGGUGCmUsmUsmUsU | |
| GuideDesign90_ABE_NGC | mAsmCsmGsCCAUGGGGAGCAAUCUCGUUUUAGAGCUAGAA | 1382 |
| _20nt_3- | AUAGCAAGUUAAAAUAAGGCUAGUCCGUUAUCAACUUGAAA | |
| 9_008_+_31946216_spCas9 | AAGUGGCACCGAGUCGGUGCmUsmUsmUSU | |
| GuideDesign90_ABE_NGC | mAsmCsmGsCCAUGGGGAGCAAUCUCGUUUUAGAGCUAGAA | 1383 |
| _20nt_3- | AUAGCAAGUUAAAAUAAGGCUAGUCCGUUAUCAACUUGAAA | |
| 12_020_+_31946216_spCas9 | AAGUGGCACCGAGUCGGUGCmUsmUsmUSU | |
| GuideDesign90_ABE_NG | mAsmCsmGsCCAUGGGGAGCAAUCUCGUUUUAGAGCUAGAA | 1384 |
| _20nt_3- | AUAGCAAGUUAAAAUAAGGCUAGUCCGUUAUCAACUUGAAA | |
| 9_027_+_31946216_spCas9 | AAGUGGCACCGAGUCGGUGCmUsmUsmUsU | |
| GuideDesign90_ABE_NRN | mAsmCsmGsCCAUGGGGAGCAAUCUCGUUUUAGAGCUAGAA | 1385 |
| _20nt_3- | AUAGCAAGUUAAAAUAAGGCUAGUCCGUUAUCAACUUGAAA | |
| 9_021_+_31946216_spCas9 | AAGUGGCACCGAGUCGGUGCmUsmUsmUsU | |
| GuideDesign90_MBE_NRN | mAsmCsmGsCCAUGGGGAGCAAUCUCGUUUUAGAGCUAGAA | 1386 |
| _20nt_3- | AUAGCAAGUUAAAAUAAGGCUAGUCCGUUAUCAACUUGAAA | |
| 9_023_+_31946216_spCas9 | AAGUGGCACCGAGUCGGUGCmUsmUsmUsU | |
| GuideDesign90_ABE_NRCH | mAsmCsmGsCCAUGGGGAGCAAUCUCGUUUUAGAGCUAGAA | 1387 |
| _20nt_3- | AUAGCAAGUUAAAAUAAGGCUAGUCCGUUAUCAACUUGAAA | |
| 9_024_+_31946216_spCas9 | AAGUGGCACCGAGUCGGUGCmUsmUsmUsU | |
| GuideDesign90_CBE_NGA | mAsmGsmAsUUGCUCCCCAUGGCGUUGUUUUAGAGCUAGAA | 1388 |
| _20nt_3-16_035_−_ | AUAGCAAGUUAAAAUAAGGCUAGUCCGUUAUCAACUUGAAA | |
| 31946212_spCas9 | AAGUGGCACCGAGUCGGUGCmUsmUsmUsU | |
| GuideDesign90_ABE_VTTN | mGsmUsmUsCUGUCUUUUGGUCAGGACAACCGUCUAGCUAU | 1389 |
| 22nt_5- | AAGUGCUGCAGGGUGUGAGAAACUCCUAUUGCUGGACGAUG | |
| 9_017_+_31946210_cas12b | UCUCUUACGAGGCAUUAGCACCAACGCCAUGGGGAGCAAUS | |
| | mCsmUsmC | |
| GuideDesign90_ABE_NG | mCsmCsmAsUGGCGUUGGAAGGCAGGGUUUUAGAGCUAGAA | 1390 |
| _20nt_3-9_027_−_ | AUAGCAAGUUAAAAUAAGGCUAGUCCGUUAUCAACUUGAAA | |
| 31946203_spCas9 | AAGUGGCACCGAGUCGGUGCmUsmUsmUsU | |
| GuideDesign90_ABE_NRN | mCsmCsmAsUGGCGUUGGAAGGCAGGGUUUUAGAGCUAGAA | 1391 |
| _20nt_3-9_021_−_ | AUAGCAAGUUAAAAUAAGGCUAGUCCGUUAUCAACUUGAAA | |
| 31946202_spCas9 | AAGUGGCACCGAGUCGGUGCmUsmUsmUsU | |
| GuideDesign90_MBE_NRN | mCsmCsmAsUGGCGUUGGAAGGCAGGGUUUUAGAGCUAGAA | 1392 |
| _20nt_3-9_023_−_ | AUAGCAAGUUAAAAUAAGGCUAGUCCGUUAUCAACUUGAAA | |
| 31946202_spCas9 | AAGUGGCACCGAGUCGGUGCmUsmUsmUsU | |
| GuideDesign90_ABE_NGA | mCsmCsmAsUGGCGUUGGAAGGCAGGGUUUUAGAGCUAGAA | 1393 |
| _20nt_3-12_019_−_ | AUAGCAAGUUAAAAUAAGGCUAGUCCGUUAUCAACUUGAAA | |
| 31946202_spCas9 | AAGUGGCACCGAGUCGGUGCmUsmUsmUSU | |
| GuideDesign90_ABE_NRN | mCsmCsmCsAUGGCGUUGGAAGGCAGGUUUUAGAGCUAGAA | 1394 |
| _20nt_3-9_021_−_ | AUAGCAAGUUAAAAUAAGGCUAGUCCGUUAUCAACUUGAAA | |
| 31946203_spCas9 | AAGUGGCACCGAGUCGGUGCmUsmUsmUsU | |
| GuideDesign90_CBE_NRN | mCsmCsmCsAUGGCGUUGGAAGGCAGGUUUUAGAGCUAGAA | 1395 |
| _20nt_3-9_022_−_ | AUAGCAAGUUAAAAUAAGGCUAGUCCGUUAUCAACUUGAAA | |
| 31946203_spCas9 | AAGUGGCACCGAGUCGGUGCmUsmUsmUsU | |
| GuideDesign90_MBE_NRN | mCsmCsmCsAUGGCGUUGGAAGGCAGGUUUUAGAGCUAGAA | 1396 |
| _20nt_3-9_023_−_ | AUAGCAAGUUAAAAUAAGGCUAGUCCGUUAUCAACUUGAAA | |
| 31946203_spCas9 | AAGUGGCACCGAGUCGGUGCmUsmUsmUsU | |
| GuideDesign90_CBE_NRRH | mCsmCsmCsAUGGCGUUGGAAGGCAGGUUUUAGAGCUAGAA | 1397 |
| _20nt_3-9_031_−_ | AUAGCAAGUUAAAAUAAGGCUAGUCCGUUAUCAACUUGAAA | |
| 31946202_spCas9 | AAGUGGCACCGAGUCGGUGCmUsmUsmUsU | |
| GuideDesign90_CBE_NGA | mCsmCsmCsCAUGGCGUUGGAAGGCAGUUUUAGAGCUAGAA | 1398 |
| _20nt_3-16_035_−_ | AUAGCAAGUUAAAAUAAGGCUAGUCCGUUAUCAACUUGAAA | |
| 31946204_spCas9 | AAGUGGCACCGAGUCGGUGCmUsmUsmUsU | |
| GuideDesign90_ABE_NG | mCsmCsmCsCAUGGCGUUGGAAGGCAGUUUUAGAGCUAGAA | 1399 |
| _20nt_3-9_027_−_ | AUAGCAAGUUAAAAUAAGGCUAGUCCGUUAUCAACUUGAAA | |
| 31946205_spCas9 | AAGUGGCACCGAGUCGGUGCmUsmUsmUsU | |
| GuideDesign90_CBE_NG | mCsmCsmCsCAUGGCGUUGGAAGGCAGUUUUAGAGCUAGAA | 1400 |
| _20nt_4-9_028_−_ | AUAGCAAGUUAAAAUAAGGCUAGUCCGUUAUCAACUUGAAA | |
| 31946205_spCas9 | AAGUGGCACCGAGUCGGUGCmUsmUsmUsU | |
| GuideDesign90_ABE_NRN | mCsmCsmCsCAUGGCGUUGGAAGGCAGUUUUAGAGCUAGAA | 1401 |
| _20nt_3-9_021_−_ | AUAGCAAGUUAAAAUAAGGCUAGUCCGUUAUCAACUUGAAA | |
| 31946204_spCas9 | AAGUGGCACCGAGUCGGUGCmUsmUsmUsU | |
| GuideDesign90_CBE_NRN | mCsmCsmCsCAUGGCGUUGGAAGGCAGUUUUAGAGCUAGAA | 1402 |
| _20nt_3-9_022_−_ | AUAGCAAGUUAAAAUAAGGCUAGUCCGUUAUCAACUUGAAA | |
| 31946204_spCas9 | AAGUGGCACCGAGUCGGUGCmUsmUsmUsU | |
| GuideDesign90_MBE_NRN | mCsmCsmCsCAUGGCGUUGGAAGGCAGUUUUAGAGCUAGAA | 1403 |
| _20nt_3-9_023_−_ | AUAGCAAGUUAAAAUAAGGCUAGUCCGUUAUCAACUUGAAA | |
| 31946204_spCas9 | AAGUGGCACCGAGUCGGUGCmUsmUsmUsU | |
| GuideDesign90_ABE_NGA | mCsmCsmCsCAUGGCGUUGGAAGGCAGUUUUAGAGCUAGAA | 1404 |
| _20nt_3-12_019_−_ | AUAGCAAGUUAAAAUAAGGCUAGUCCGUUAUCAACUUGAAA | |
| 31946204_spCas9 | AAGUGGCACCGAGUCGGUGCmUsmUsmUsU | |
| GuideDesign90_CBE_NGA | mCsmCsmCsCAUGGCGUUGGAAGGCAGUUUUAGAGCUAGAA | 1405 |
| _20nt_4-9_006_−_ | AUAGCAAGUUAAAAUAAGGCUAGUCCGUUAUCAACUUGAAA | |
| 31946204_spCas9 | AAGUGGCACCGAGUCGGUGCmUsmUsmUsU | |
| GuideDesign90_ABE_NRN | mCsmUsmCsCCCAUGGCGUUGGAAGGGUUUUAGAGCUAGAA | 1406 |
| _20nt_3-9_021_−_ | AUAGCAAGUUAAAAUAAGGCUAGUCCGUUAUCAACUUGAAA | |
| 31946206_spCas9 | AAGUGGCACCGAGUCGGUGCmUsmUsmUsU | |
| GuideDesign90_CBE_NRN | mCsmUsmCsCCCAUGGCGUUGGAAGGGUUUUAGAGCUAGAA | 1407 |
| _20nt_3-9_022_−_ | AUAGCAAGUUAAAAUAAGGCUAGUCCGUUAUCAACUUGAAA | |
| 31946206_spCas9 | AAGUGGCACCGAGUCGGUGCmUsmUsmUsU | |
| GuideDesign90_MBE_NRN | mCsmUsmCsCCCAUGGCGUUGGAAGGGUUUUAGAGCUAGAA | 1408 |
| _20nt_3-9_023_−_ | AUAGCAAGUUAAAAUAAGGCUAGUCCGUUAUCAACUUGAAA | |
| 31946206_spCas9 | AAGUGGCACCGAGUCGGUGCmUsmUsmUsU | |
| GuideDesign90_ABE_VTTN | mGsmUsmUsCUGUCUUUUGGUCAGGACAACCGUCUAGCUAU | 1409 |
| 22nt_5-9_017_−_ | AAGUGCUGCAGGGUGUGAGAAACUCCUAUUGCUGGACGAUG | |
| 31946207_cas12b | UCUCUUACGAGGCAUUAGCACCUCCCCAUGGCGUUGGAAGS | |
| | mGsmCsmA | |
| GuideDesign90_CBE_NGG | mGsmAsmGsAUUGCUCCCCAUGGCGUGUUUUAGAGCUAGAA | 1410 |
| _20nt_3-16_033_−_ | AUAGCAAGUUAAAAUAAGGCUAGUCCGUUAUCAACUUGAAA | |
| 31946213_SpCas9 | AAGUGGCACCGAGUCGGUGCmUsmUsmUSU | |
| GuideDesign90_ABE_NGG | mUsmCsmCsCCAUGGCGUUGGAAGGCGUUUUAGAGCUAGAA | 1411 |
| _20nt_3-9_002_−_ | AUAGCAAGUUAAAAUAAGGCUAGUCCGUUAUCAACUUGAAA | |
| 31946205_spCas9 | AAGUGGCACCGAGUCGGUGCmUsmUsmUSU | |
| GuideDesign90_ABE_NG | mUsmCsmCsCCAUGGCGUUGGAAGGCGUUUUAGAGCUAGAA | 1412 |
| _20nt_3-9_027_−_ | AUAGCAAGUUAAAAUAAGGCUAGUCCGUUAUCAACUUGAAA | |
| 31946206_spCas9 | AAGUGGCACCGAGUCGGUGCmUsmUsmUsU | |
| GuideDesign90_CBE_NG | mUsmCsmCsCCAUGGCGUUGGAAGGCGUUUUAGAGCUAGAA | 1413 |
| _20nt_4-9_028_−_ | AUAGCAAGUUAAAAUAAGGCUAGUCCGUUAUCAACUUGAAA | |
| 31946206_spCas9 | AAGUGGCACCGAGUCGGUGCmUsmUsmUSU | |
| GuideDesign90_ABE_NGG | mUsmCsmCsCCAUGGCGUUGGAAGGCGUUUUAGAGCUAGAA | 1414 |
| _20nt_3-12_018_−_ | AUAGCAAGUUAAAAUAAGGCUAGUCCGUUAUCAACUUGAAA | |
| 31946205_spCas9 | AAGUGGCACCGAGUCGGUGCmUsmUsmUsU | |
| GuideDesign90_ABE_NRN | mUsmCsmCsCCAUGGCGUUGGAAGGCGUUUUAGAGCUAGAA | 1415 |
| _20nt_3-9_021_−_ | AUAGCAAGUUAAAAUAAGGCUAGUCCGUUAUCAACUUGAAA | |
| 31946205_spCas9 | AAGUGGCACCGAGUCGGUGCmUsmUsmUsU | |
| GuideDesign90_CBE_NRN | mUsmCsmCsCCAUGGCGUUGGAAGGCGUUUUAGAGCUAGAA | 1416 |
| _20nt_3-9_022_−_ | AUAGCAAGUUAAAAUAAGGCUAGUCCGUUAUCAACUUGAAA | |
| 31946205_spCas9 | AAGUGGCACCGAGUCGGUGCmUsmUsmUsU | |
| GuideDesign90_MBE_NRN | mUsmCsmCsCCAUGGCGUUGGAAGGCGUUUUAGAGCUAGAA | 1417 |
| _20nt_3-9_023_−_ | AUAGCAAGUUAAAAUAAGGCUAGUCCGUUAUCAACUUGAAA | |
| 31946205_spCas9 | AAGUGGCACCGAGUCGGUGCmUsmUsmUSU | |
| GuideDesign90_CBE_NRRH | mUsmCsmCsCCAUGGCGUUGGAAGGCGUUUUAGAGCUAGAA | 1418 |
| _20nt_3-9_031_−_ | AUAGCAAGUUAAAAUAAGGCUAGUCCGUUAUCAACUUGAAA | |
| 31946204_spCas9 | AAGUGGCACCGAGUCGGUGCmUsmUsmUSU | |
| GuideDesign90_CBE_NGG | mUsmCsmCsCCAUGGCGUUGGAAGGCGUUUUAGAGCUAGAA | 1419 |
| _20nt_4-9_003_−_ | AUAGCAAGUUAAAAUAAGGCUAGUCCGUUAUCAACUUGAAA | |
| 31946205_spCas9 | AAGUGGCACCGAGUCGGUGCmUsmUsmUsU | |
| GuideDesign90_CBE_NGG | mUsmCsmCsCCAUGGCGUUGGAAGGCGUUUUAGAGCUAGAA | 1420 |
| _20nt_3-9_029_−_ | AUAGCAAGUUAAAAUAAGGCUAGUCCGUUAUCAACUUGAAA | |
| 31946205_spCas9 | AAGUGGCACCGAGUCGGUGCmUsmUsmUSU | |
| GuideDesign90_CBE_NGG | mUsmCsmCsCCAUGGCGUUGGAAGGCGUUUUAGAGCUAGAA | 1421 |
| _20nt_3-16_033_−_ | AUAGCAAGUUAAAAUAAGGCUAGUCCGUUAUCAACUUGAAA | |
| 31946205_SpCas9 | AAGUGGCACCGAGUCGGUGCmUsmUsmUsU | |
| GuideDesign90_ABE_NGC | mUsmGsmCsUCCCCAUGGCGUUGGAAGUUUUAGAGCUAGAA | 1422 |
| _20nt_3-12_020_−_ | AUAGCAAGUUAAAAUAAGGCUAGUCCGUUAUCAACUUGAAA | |
| 31946208_spCas9 | AAGUGGCACCGAGUCGGUGCmUsmUsmUSU | |
| GuideDesign90_CBE_NGC | mUsmGsmCsUCCCCAUGGCGUUGGAAGUUUUAGAGCUAGAA | 1423 |
| _20nt_4-9_009_−_ | AUAGCAAGUUAAAAUAAGGCUAGUCCGUUAUCAACUUGAAA | |
| 31946208_spCas9 | AAGUGGCACCGAGUCGGUGCmUsmUsmUsU | |
| GuideDesign90_ABE_NG | mUsmGsmCsUCCCCAUGGCGUUGGAAGUUUUAGAGCUAGAA | 1424 |
| _20nt_3-9_027_−_ | AUAGCAAGUUAAAAUAAGGCUAGUCCGUUAUCAACUUGAAA | |
| 31946209_spCas9 | AAGUGGCACCGAGUCGGUGCmUsmUsmUsU | |
| GuideDesign90_CBE_NG | mUsmGsmCsUCCCCAUGGCGUUGGAAGUUUUAGAGCUAGAA | 1425 |
| _20nt_4-9_028_−_ | AUAGCAAGUUAAAAUAAGGCUAGUCCGUUAUCAACUUGAAA | |
| 31946209_spCas9 | AAGUGGCACCGAGUCGGUGCmUsmUsmUsU | |
| GuideDesign90_ABE_NRN | mUsmGsmCsUCCCCAUGGCGUUGGAAGUUUUAGAGCUAGAA | 1426 |
| _20nt_3-9_021_−_ | AUAGCAAGUUAAAAUAAGGCUAGUCCGUUAUCAACUUGAAA | |
| 31946208_spCas9 | AAGUGGCACCGAGUCGGUGCmUsmUsmUsU | |
| GuideDesign90_CBE_NRN | mUsmGsmCsUCCCCAUGGCGUUGGAAGUUUUAGAGCUAGAA | 1427 |
| _20nt_3-9_022_−_ | AUAGCAAGUUAAAAUAAGGCUAGUCCGUUAUCAACUUGAAA | |
| 31946208_spCas9 | AAGUGGCACCGAGUCGGUGCmUsmUsmUsU | |
| GuideDesign90_MBE_NRN | mUsmGsmCsUCCCCAUGGCGUUGGAAGUUUUAGAGCUAGAA | 1428 |
| _20nt_3-9_023_−_ | AUAGCAAGUUAAAAUAAGGCUAGUCCGUUAUCAACUUGAAA | |
| 31946208_spCas9 | AAGUGGCACCGAGUCGGUGCmUsmUsmUsU | |
| GuideDesign90_ABE_NRCH | mUsmGsmCsUCCCCAUGGCGUUGGAAGUUUUAGAGCUAGAA | 1429 |
| _20nt_3-9_024_−_ | AUAGCAAGUUAAAAUAAGGCUAGUCCGUUAUCAACUUGAAA | |
| 31946207_spCas9 | AAGUGGCACCGAGUCGGUGCmUsmUsmUsU | |
| GuideDesign90_CBE_NRCH | mUsmGsmCsUCCCCAUGGCGUUGGAAGUUUUAGAGCUAGAA | 1430 |
| _20nt_3-9_030_−_ | AUAGCAAGUUAAAAUAAGGCUAGUCCGUUAUCAACUUGAAA | |
| 31946207_spCas9 | AAGUGGCACCGAGUCGGUGCmUsmUsmUsU | |
| GuideDesign90_CBE_NG | mUsmUsmGsCUCCCCAUGGCGUUGGAGUUUUAGAGCUAGAA | 1431 |
| _20nt_4-9_028_−_ | AUAGCAAGUUAAAAUAAGGCUAGUCCGUUAUCAACUUGAAA | |
| 31946210_spCas9 | AAGUGGCACCGAGUCGGUGCmUsmUsmUSU | |
| GuideDesign90_CBE_NRN | mUsmUsmGsCUCCCCAUGGCGUUGGAGUUUUAGAGCUAGAA | 1432 |
| _20nt_3-9_022_−_ | AUAGCAAGUUAAAAUAAGGCUAGUCCGUUAUCAACUUGAAA | |
| 31946209_spCas9 | AAGUGGCACCGAGUCGGUGCmUsmUsmUsU | |
| GuideDesign90_MBE_NRN | mUsmUsmGsCUCCCCAUGGCGUUGGAGUUUUAGAGCUAGAA | 1433 |
| _20nt_3-9_023_−_ | AUAGCAAGUUAAAAUAAGGCUAGUCCGUUAUCAACUUGAAA | |
| 31946209_spCas9 | AAGUGGCACCGAGUCGGUGCmUsmUsmUSU | |
| GuideDesign90_CBE_NRRH | mUsmUsmGsCUCCCCAUGGCGUUGGAGUUUUAGAGCUAGAA | 1434 |
| _20nt_3-9_031_−_ | AUAGCAAGUUAAAAUAAGGCUAGUCCGUUAUCAACUUGAAA | |
| 31946208_spCas9 | AAGUGGCACCGAGUCGGUGCmUsmUsmUsU | |
| GuideDesign90_CBE_NGG | mUsmUsmGsCUCCCCAUGGCGUUGGAGUUUUAGAGCUAGAA | 1435 |
| _20nt_4-9_003_−_ | AUAGCAAGUUAAAAUAAGGCUAGUCCGUUAUCAACUUGAAA | |
| 31946209_spCas9 | AAGUGGCACCGAGUCGGUGCmUsmUsmUsU | |
| GuideDesign90_CBE_NGG | mUsmUsmGsCUCCCCAUGGCGUUGGAGUUUUAGAGCUAGAA | 1436 |
| _20nt_3-9_029_−_ | AUAGCAAGUUAAAAUAAGGCUAGUCCGUUAUCAACUUGAAA | |
| 31946209_spCas9 | AAGUGGCACCGAGUCGGUGCmUsmUsmUsU | |
| GuideDesign90_CBE_NGG | mUsmUsmGsCUCCCCAUGGCGUUGGAGUUUUAGAGCUAGAA | 1437 |
| _20nt_3-16_033_−_ | AUAGCAAGUUAAAAUAAGGCUAGUCCGUUAUCAACUUGAAA | |
| 31946209_SpCas9 | AAGUGGCACCGAGUCGGUGCmUsmUsmUsU | |

⁶“mN” indicates a 2′-OMe modification of the nucleotide “N”, and “Ns” indicates that the nucleotide “N” is linked to the following (i.e., 3′) nucleotide by a phosphorothioate (PS).

TABLE 1G
Representative sequences for guide polynucleotides for use in guiding a base
editor to alter a start codon or TATA box of a complement factor B polynucleotide
using base editing.⁷

| Guide | | SEQ |
|---|---|---|
| polynucleotide | | ID |
| name | Guide polynucleotide sequence | NO |
| gRNA3642 | mCsmAsmGsAACACAGAUUGUAUAAAGUUUUAGAGCUAGAAAUAGCAAGU | 1438 |
| | UAAAAUAAGGCUAGUCCGUUAUCAACUUGAAAAAGUGGCACCGAGUCGGU | |
| | GCmUsmUsmUsU | |
| gRNA3643 | mAsmCsmAsCAGAUUGUAUAAAAGGCGUUUUAGAGCUAGAAAUAGCAAGU | 1439 |
| | UAAAAUAAGGCUAGUCCGUUAUCAACUUGAAAAAGUGGCACCGAGUCGGU | |
| | GCmUsmUsmUsU | |
| gRNA3644 | mCsmAsmCsAGAUUGUAUAAAAGGCUGUUUUAGAGCUAGAAAUAGCAAGU | 1440 |
| | UAAAAUAAGGCUAGUCCGUUAUCAACUUGAAAAAGUGGCACCGAGUCGGU | |
| | GCmUsmUsmUsU | |
| gRNA3645 | mAsmCsmAsGAUUGUAUAAAAGGCUGGUUUUAGAGCUAGAAAUAGCAAGU | 1441 |
| | UAAAAUAAGGCUAGUCCGUUAUCAACUUGAAAAAGUGGCACCGAGUCGGU | |
| | GCmUsmUsmUsU | |
| gRNA3646 | mCsmAsmGsAUUGUAUAAAAGGCUGGGUUUUAGAGCUAGAAAUAGCAAGU | 1442 |
| | UAAAAUAAGGCUAGUCCGUUAUCAACUUGAAAAAGUGGCACCGAGUCGGU | |
| | GCmUsmUsmUsU | |
| gRNA3647 | mUsmAsmUsAAAAGGCUGGGGGCUGGGUUUUAGAGCUAGAAAUAGCAAGU | 1443 |
| | UAAAAUAAGGCUAGUCCGUUAUCAACUUGAAAAAGUGGCACCGAGUCGGU | |
| | GCmUsmUsmUsU | |
| gRNA3648 | mAsmUsmAsAAAGGCUGGGGGCUGGUGUUUUAGAGCUAGAAAUAGCAAGU | 1444 |
| | UAAAAUAAGGCUAGUCCGUUAUCAACUUGAAAAAGUGGCACCGAGUCGGU | |
| | GCmUsmUsmUsU | |
| gRNA3649 | mUsmAsmAsAAGGCUGGGGGCUGGUGGUUUUAGAGCUAGAAAUAGCAAGU | 1445 |
| | UAAAAUAAGGCUAGUCCGUUAUCAACUUGAAAAAGUGGCACCGAGUCGGU | |
| | GCmUsmUsmUsU | |
| gRNA3650 | mCsmUsmUsUUAUACAAUCUGUGUUCGUUUUAGAGCUAGAAAUAGCAAGU | 1446 |
| | UAAAAUAAGGCUAGUCCGUUAUCAACUUGAAAAAGUGGCACCGAGUCGGU | |
| | GCmUsmUsmUsU | |
| gRNA3651 | mAsmAsmUsCUGUGUUCUGGCACCUGGUUUUAGAGCUAGAAAUAGCAAGU | 1447 |
| | UAAAAUAAGGCUAGUCCGUUAUCAACUUGAAAAAGUGGCACCGAGUCGGU | |
| | GCmUsmUsmUsU | |
| gRNA3652 | mAsmAsmAsAGGCUGGGGGCUGGUGGGUUUUAGAGCUAGAAAUAGCAAGU | 1448 |
| | UAAAAUAAGGCUAGUCCGUUAUCAACUUGAAAAAGUGGCACCGAGUCGGU | |
| | GCmUsmUsmUsU | |
| gRNA3653 | mAsmGsmAsACACAGAUUGUAUAAAAGUUUUAGAGCUAGAAAUAGCAAGU | 1449 |
| | UAAAAUAAGGCUAGUCCGUUAUCAACUUGAAAAAGUGGCACCGAGUCGGU | |
| | GCmUsmUsmUsU | |
| gRNA3654 | mAsmUsmCsUGUGUUCUGGCACCUGCGUUUUAGAGCUAGAAAUAGCAAGU | 1450 |
| | UAAAAUAAGGCUAGUCCGUUAUCAACUUGAAAAAGUGGCACCGAGUCGGU | |
| | GCmUsmUsmUsU | |
| gRNA3655 | mAsmGsmAsUUGUAUAAAAGGCUGGGGGUUUUAGUACUCUGUAAUGAAAA | 1451 |
| | UUACAGAAUCUACUAAAACAAGGCAAAAUGCCGUGUUUAUCUCGUCAACU | |
| | UGUUGGCGAGAUsmUsmUsmU | |
| gRNA3656 | mCsmCsmAsCCAGCCCCCAGCCUUUUAGUUUUAGUACUCUGUAAUGAAAA | 1452 |
| | UUACAGAAUCUACUAAAACAAGGCAAAAUGCCGUGUUUAUCUCGUCAACU | |
| | UGUUGGCGAGAUsmUsmUsmU | |
| gRNA3657 | mUsmGsmCsUCCCCAUGGCGUUGGAAGUUUUAGAGCUAGAAAUAGCAAGU | 1453 |
| | UAAAAUAAGGCUAGUCCGUUAUCAACUUGAAAAAGUGGCACCGAGUCGGU | |
| | GCmUsmUsmUsU | |
| gRNA3658 | mUsmUsmGsCUCCCCAUGGCGUUGGAGUUUUAGAGCUAGAAAUAGCAAGU | 1454 |
| | UAAAAUAAGGCUAGUCCGUUAUCAACUUGAAAAAGUGGCACCGAGUCGGU | |
| | GCmUsmUsmUsU | |
| gRNA3659 | mGsmAsmGsAUUGCUCCCCAUGGCGUGUUUUAGAGCUAGAAAUAGCAAGU | 1455 |
| | UAAAAUAAGGCUAGUCCGUUAUCAACUUGAAAAAGUGGCACCGAGUCGGU | |
| | GCmUsmUsmUsU | |
| gRNA3660 | mCsmCsmCsCAUGGCGUUGGAAGGCAGUUUUAGAGCUAGAAAUAGCAAGU | 1456 |
| | UAAAAUAAGGCUAGUCCGUUAUCAACUUGAAAAAGUGGCACCGAGUCGGU | |
| | GCmUsmUsmUsU | |
| gRNA3661 | mCsmCsmAsUGGCGUUGGAAGGCAGGGUUUUAGAGCUAGAAAUAGCAAGU | 1457 |
| | UAAAAUAAGGCUAGUCCGUUAUCAACUUGAAAAAGUGGCACCGAGUCGGU | |
| | GCmUsmUsmUsU | |
| gRNA3662 | mAsmGsmAsUUGCUCCCCAUGGCGUUGUUUUAGAGCUAGAAAUAGCAAGU | 1458 |
| | UAAAAUAAGGCUAGUCCGUUAUCAACUUGAAAAAGUGGCACCGAGUCGGU | |
| | GCmUsmUsmUsU | |

⁷“mN” indicates a 2′-OMe modification of the nucleotide “N”, and “Ns” indicates that the nucleotide “N” is linked to the following (i.e., 3′) nucleotide by a phosphorothioate (PS).

TABLE 1H
Representative sequences for guide polynucleotides for use in guiding a base
editor to alter a complement factor B splice site.⁸

| Target | Guide | Nucleotide | | SEQ |
|---|---|---|---|---|
| Site | polynucleotide | Modification | | ID |
| Name | name | Scheme | Guide polynucleotide sequence | NO |
| TSBTx38 | gRNA1 | End Mods | mCsmCsmUsCAGAUGUCUAUGUGUUUGUUUUA | 1459 |
| 26 | 193 | | GAGCUAGAAAUAGCAAGUUAAAAUAAGGCUAG | |
| | | | UCCGUUAUCAACUUGAAAAAGUGGCACCGAGU | |
| | | | CGGUGCUsmUsmUsmU | |
| TSBTx38 | gRNA2 | HM01 | mCsmCsmUsCAGAUGUCUAUGUGUUUGUUUUA | 1460 |
| 26 | 210 | | GAmGmCmUmAmGmAmAmAmUmAmGmCAAGUUA | |
| | | | AAAUAAGGCUAGUCCGUUAUCAmAmCmUmUmG | |
| | | | mAmAmAmAmAmGmUmGmGmCmAmCmCmGmAmG | |
| | | | mUmCmGmGmUmGmCmUsmUsmUsmU | |
| TSBTx38 | gRNA2 | HM07 | mCsmCsmUsmCmAmGmAmUmGmUCUAUGUGUU | 1461 |
| 26 | 444 | | UmGUUUUAGmAmGmCmUmAmGmAmAmAmUmAm | |
| | | | GmCmAmAGUUmAAmAAmUAmAmGmGmCmUmAG | |
| | | | UmCmCGUUAmUmCAAmCmUmUmGmAmAmAmAm | |
| | | | AmGmUmGGmCmAmCmCmGmAmGmUmCmGmGmU | |
| | | | mGmCmUsmUsmUsmU | |
| TSBTx38 | gRNA2 | NLS | mCsmCsmUsCAGAUGUCUAUGUGUUUGUUUUA | 1462; |
| 26 | 445 | (bpsv40) | GAGCUAGAAAUAGCAAGUUAAAAUAAGGCUAG | 1463 |
| | | | UCCGUUAUCAACUUGAAAAAGUGGCACCGAGU | |
| | | | CGGUGCmUsmUsmUsU-NHC6-CrossL-ac- | |
| | | | CKRTADGSEFESPKKKRKV | |
| TSBTx38 | gRNA2 | Hairpin | mCsmCsmUsCAGAUGUCUAUGUGUUUGUUUUA | 1464 |
| 26 | 446 | Extension | GAmGmCmCmGmGmCmGmGmAmAmAmCmGmCmC | |
| | | (LONGEST- | mGmGmCAAGUUAAAAUAAGGCUAGUCCGUUAU | |
| | | OLD) | CAmAmCmUmUmGmAmAmAmAmAmGUGmGmCmA | |
| | | | mCmCmGmAmGmUmCmGmGmUmGmCmUsmUsmU | |
| | | | smU | |
| TSBTx38 | gRNA3 | LONGEST | mCsmCsmUsCAGAUGUCUAUGUGUUUGUUUUA | 1465 |
| 26 | 335 | | GAmGmCmCmGmGmCmGmGmAmAmAmCmGmCmC | |
| | | | mGmGmCAAGUUAAAAUAAGGCUAGUCCGUUAU | |
| | | | CAmAmCmUmUmGmAmAmAmAmAmGmUmGmGmC | |
| | | | mAmCmCmGmAmGmUmCmGmGmUmGmCmUsmUs | |
| | | | mUsmU | |
| TSBTx38 | gRNA3 | LONGEST + | mCsmCsmUsCAGAUGUCUAUGUGUUUGUUUUA | 1466; |
| 26 | 336 | NLS | GAmGmCmCmGmGmCmGmGmAmAmAmCmGmCmC | 1463 |
| | | | mGmGmCAAGUUAAAAUAAGGCUAGUCCGUUAU | |
| | | | CAmAmCmUmUmGmAmAmAmAmAmGmUmGmGmC | |
| | | | mAmCmCmGmAmGmUmCmGmGmUmGmCmUsmUs | |
| | | | mUsmU-NHC6-CrossL- | |
| | | | CKRTADGSEFESPKKKRKV | |
| TSBTx38 | gRNA1 | End Mods | mUsmCsmUsCACCUCUGCAAGUAUUGGUUUUA | 1467 |
| 35 | 202 | | GAGCUAGAAAUAGCAAGUUAAAAUAAGGCUAG | |
| | | | UCCGUUAUCAACUUGAAAAAGUGGCACCGAGU | |
| | | | CGGUGCUsmUsmUsmU | |
| TSBTx38 | gRNA2 | HM01 | mUsmCsmUsCACCUCUGCAAGUAUUGGUUUUA | 1468 |
| 35 | 447 | | GAmGmCmUmAmGmAmAmAmUmAmGmCAAGUUA | |
| | | | AAAUAAGGCUAGUCCGUUAUCAmAmCmUmUmG | |
| | | | mAmAmAmAmAmGmUmGmGmCmAmCmCmGmAmG | |
| | | | mUmCmGmGmUmGmCmUsmUsmUsmU | |
| TSBTx38 | gRNA2 | HM07 | mUsmCsmUsmCmAmCmCmUmCmUGCAAGUAUU | 1469 |
| 35 | 448 | | GmGUUUUAGmAmGmCmUmAmGmAmAmAmUmAm | |
| | | | GmCmAmAGUUmAAmAAmUAmAmGmGmCmUmAG | |
| | | | UmCmCGUUAmUmCAAmCmUmUmGmAmAmAmAm | |
| | | | AmGmUmGGmCmAmCmCmGmAmGmUmCmGmGmU | |
| | | | mGmCmUsmUsmUsmU | |
| TSBTx38 | gRNA2 | NLS | mUsmCsmUsCACCUCUGCAAGUAUUGGUUUUA | 1470; |
| 35 | 449 | (bpsv40) | GAGCUAGAAAUAGCAAGUUAAAAUAAGGCUAG | 1463 |
| | | | UCCGUUAUCAACUUGAAAAAGUGGCACCGAGU | |
| | | | CGGUGCmUsmUsmUsU-NHC6-CrossL-ac- | |
| | | | CKRTADGSEFESPKKKRKV | |
| TSBTx38 | gRNA2 | Hairpin | mUsmCsmUsCACCUCUGCAAGUAUUGGUUUUA | 1471 |
| 35 | 450 | Extension | GAmGmCmCmGmGmCmGmGmAmAmAmCmGmCmC | |
| | | (LONGEST- | mGmGmCAAGUUAAAAUAAGGCUAGUCCGUUAU | |
| | | OLD) | CAmAmCmUmUmGmAmAmAmAmAmGUGmGmCmA | |
| | | | mCmCmGmAmGmUmCmGmGmUmGmCmUsmUsmU | |
| | | | smU | |
| TSBTx38 | gRNA3 | LONGEST | mUsmCsmUsCACCUCUGCAAGUAUUGGUUUUA | 1472 |
| 35 | 337 | | GAmGmCmCmGmGmCmGmGmAmAmAmCmGmCmC | |
| | | | mGmGmCAAGUUAAAAUAAGGCUAGUCCGUUAU | |
| | | | CAmAmCmUmUmGmAmAmAmAmAmGmUmGmGmC | |
| | | | mAmCmCmGmAmGmUmCmGmGmUmGmCmUsmUs | |
| | | | mUsmU | |
| TSBTx38 | gRNA3 | LONGEST + | mUsmGsmUsCACCUCUGCAAGUAUUGGUUUUA | 1473; |
| 35 | 338 | NLS | GAmGmCmCmGmGmCmGmGmAmAmAmCmGmCmC | 1463 |
| | | | mGmGmCAAGUUAAAAUAAGGCUAGUCCGUUAU | |
| | | | CAmAmCmUmUmGmAmAmAmAmAmGmUmGmGmC | |
| | | | mAmCmCmGmAmGmUmCmGmGmUmGmCmUsmUs | |
| | | | mUsmU-NHC5-CrossL- | |
| | | | CKRTADGSEFESPKKKRKV | |
| TSBTx38 | gRNA2 | End Mods | mUsmGsmCsUUACAAUGACUGAGAUCUGUUUU | 1474 |
| 37 | 067 | | AGAGCUAGAAAUAGCAAGUUAAAAUAAGGCUA | |
| | | | GUCCGUUAUCAACUUGAAAAAGUGGCACCGAG | |
| | | | UCGGUGCUsmUsmUsmU | |
| TSBTx38 | gRNA2 | HM01 | mUsmGsmCsUUACAAUGACUGAGAUCUGUUUU | 1475 |
| 37 | 451 | | AGAmGmCmUmAmGmAmAmAmUmAmGmCAAGUU | |
| | | | AAAAUAAGGCUAGUCCGUUAUCAmAmCmUmUm | |
| | | | GmAmAmAmAmAmGmUmGmGmCmAmCmCmGmAm | |
| | | | GmUmCmGmGmUmGmCmUsmUsmUsmU | |
| TSBTx38 | gRNA2 | HM07 | mUsmGsmCsmUmUmAmCmAmAmUmGACUGAGA | 1476 |
| 37 | 452 | | UCUmGUUUUAGmAmGmCmUmAmGmAmAmAmUm | |
| | | | AmGmCmAmAGUUmAAmAAmUAmAmGmGmCmUm | |
| | | | AGUmCmCGUUAmUmCAAmCmUmUmGmAmAmAm | |
| | | | AmAmGmUmGGmCmAmCmCmGmAmGmUmCmGmG | |
| | | | mUmGmCmUsmUsmUsmU | |
| TSBTx38 | gRNA2 | NLS | mUsmGsmCsUUACAAUGACUGAGAUCUGUUUU | 1477; |
| 37 | 453 | (bpsv40) | AGAGCUAGAAAUAGCAAGUUAAAAUAAGGCUA | 1463 |
| | | | GUCCGUUAUCAACUUGAAAAAGUGGCACCGAG | |
| | | | UCGGUGCmUsmUsmUsU-NHC6-CrossL-ac- | |
| | | | CKRTADGSEFESPKKKRKV | |
| TSBTx38 | gRNA2 | Hairpin | mUsmGsmCsUUACAAUGACUGAGAUCUGUUUU | 1478 |
| 37 | 454 | Extension | AGAmGmCmCmGmGmCmGmGmAmAmAmCmGmCm | |
| | | (LONGEST- | CmGmGmCAAGUUAAAAUAAGGCUAGUCCGUUA | |
| | | OLD) | UCAmAmCmUmUmGmAmAmAmAmAmGUGmGmCm | |
| | | | AmCmCmGmAmGmUmCmGmGmUmGmCmUsmUsm | |
| | | | UsmU | |
| TSBTx38 | gRNA3 | LONGEST | mUsmGsmCsUUACAAUGACUGAGAUCUGUUUU | 1479 |
| 37 | 339 | | AGAmGmCmCmGmGmCmGmGmAmAmAmCmGmCm | |
| | | | CmGmGmCAAGUUAAAAUAAGGCUAGUCCGUUA | |
| | | | UCAmAmCmUmUmGmAmAmAmAmAmGmUmGmGm | |
| | | | CmAmCmCmGmAmGmUmCmGmGmUmGmCmUsmU | |
| | | | smUsmU | |
| TSBTx38 | gRNA3 | LONGEST + | mUsmGsmCsUUACAAUGACUGAGAUCUGUUUU | 1480; |
| 37 | 340 | NLS | AGAmGmCmCmGmGmCmGmGmAmAmAmCmGmCm | 1463 |
| | | | CmGmGmCAAGUUAAAAUAAGGCUAGUCCGUUA | |
| | | | UCAmAmCmUmUmGmAmAmAmAmAmGmUmGmGm | |
| | | | CmAmCmCmGmAmGmUmCmGmGmUmGmCmUsmU | |
| | | | smUsmU-NHC5-CrossL- | |
| | | | CKRTADGSEFESPKKKRKV | |
| TSBTx38 | gRNA2 | End Mods | mUsmGsmCsUUACAGUGACUGAGAUCUGUUUU | 1481 |
| 37 (NHP) | 072 | | AGAGCUAGAAAUAGCAAGUUAAAAUAAGGCUA | |
| | | | GUCCGUUAUCAACUUGAAAAAGUGGCACCGAG | |
| | | | UCGGUGCUsmUsmUsmU | |
| TSBTx38 | gRNA5 | HM01 | mUsmGsmCsUUACAGUGACUGAGAUCUGUUUU | 1482 |
| 37 (NHP) | 856 | | AGAmGmCmUmAmGmAmAmAmUmAmGmCAAGUU | |
| | | | AAAAUAAGGCUAGUCCGUUAUCAmAmCmUmUm | |
| | | | GmAmAmAmAmAmGmUmGmGmCmAmCmCmGmAm | |
| | | | GmUmCmGmGmUmGmCmUsmUsmUsmU | |

⁸“mN” indicates a 2′-OMe modification of the nucleotide “N”, and “Ns” indicates that the nucleotide “N” is linked to the following (i.e., 3′) nucleotide by a phosphorothioate (PS).

TABLE 1I
Alternative names for guide polynucleotides.

| Guide Name | Alternative Guide Name |
|---|---|
| gRNA1193 | gRNA1193-1862 |
| gRNA2210 | gRNA1193-1863 |
| gRNA2444 | gRNA2444-1992 |
| gRNA2445 | gRNA2445-1993 |
| gRNA2446 | gRNA2446-1994 |
| gRNA3335 | gRNA3335-3047 |
| gRNA3336 | gRNA3336-3048 |
| gRNA1202 | gRNA1202-1995 |
| gRNA2447 | gRNA2447-4112 |
| gRNA2448 | gRNA2448-4113 |
| gRNA2449 | gRNA2449-4114 |
| gRNA3337 | gRNA3337-3049 |
| gRNA3338 | gRNA3338-3050 |
| gRNA2067 | gRNA2067-1996 |
| gRNA2451 | gRNA2451-1997 |
| gRNA2452 | gRNA2452-1998 |
| gRNA2453 | gRNA2453-1999 |
| gRNA2454 | gRNA2454-2000 |
| gRNA3339 | gRNA3339-3051 |
| gRNA3340 | gRNA3340-3052 |

TABLE 2A
Representative spacer and target site sequences relating to disruption of a
complement factor B splice site using base editing.

| Guide | | | | |
|---|---|---|---|---|
| polynucleotide | | Target site | | SEQ ID |
| name | Alterantive name | name | Protospacer sequence | NO |
| gRNA2055 | gRNA1193_19nt | TSBTx3826 | CTCAGATGTCTATGTGTTT | 1483 |
| gRNA1193 | gRNA1193_20nt | TSBTx3826 | CCTCAGATGTCTATGTGTTT | 1484 |
| gRNA2057 | gRNA1193_21nt | TSBTx3826 | CCCTCAGATGTCTATGTGTT | 1485 |
| | | | T | |
| gRNA2058 | gRNA1193_22nt | TSBTx3826 | TCCCTCAGATGTCTATGTGT | 1486 |
| | | | TT | |
| gRNA2059 | gRNA1193_23nt | TSBT×3826 | ATCCCTCAGATGTCTATGTG | 1487 |
| | | | TTT | |
| gRNA2060 | gRNA1202_19nt | TSBTx3835 | CTCACCTCTGCAAGTATTG | 1488 |
| gRNA1202 | gRNA1202_20nt | TSBTx3835 | TCTCACCTCTGCAAGTATTG | 1489 |
| gRNA2062 | gRNA1202_21nt | TSBTx3835 | CTCTCACCTCTGCAAGTATT | 1490 |
| | | | G | |
| gRNA2063 | gRNA1202_22nt | TSBT×3835 | TCTCTCACCTCTGCAAGTAT | 1491 |
| | | | TG | |
| gRNA2064 | gRNA1202_23nt | TSBTx3835 | TTCTCTCACCTCTGCAAGTA | 1492 |
| | | | TTG | |
| gRNA2065 | gRNA1204_19nt | TSBTx3837 | CTTACAATGACTGAGATCT | 1493 |
| gRNA1204 | gRNA1204_20nt | TSBTx3837 | GCTTACAATGACTGAGATCT | 1494 |
| gRNA2067 | gRNA1204_21nt | TSBTx3837 | TGCTTACAATGACTGAGATC | 1495 |
| | | | T | |
| gRNA2068 | gRNA1204_22nt | TSBTx3837 | GTGCTTACAATGACTGAGAT | 1496 |
| | | | CT | |
| gRNA2069 | gRNA1204_23nt | TSBTx3837 | TGTGCTTACAATGACTGAGA | 1497 |
| | | | TCT | |
| gRNA2070 | gRNA1999_NHP19 | TSBTx4943 | CTTACAGTGACTGAGATCT | 1498 |
| | nt | | | |
| gRNA1999 | gRNA1999_NHP20 | TSBTx4943 | GCTTACAGTGACTGAGATCT | 1499 |
| | nt | | | |
| gRNA2072 | gRNA1999_NHP21 | TSBTx4943 | TGCTTACAGTGACTGAGATC | 1500 |
| | nt | | T | |
| gRNA2073 | gRNA1999_NHP22 | TSBTx4943 | GTGCTTACAGTGACTGAGAT | 1501 |
| | nt | | CT | |
| gRNA2074 | gRNA1999_NHP23 | TSBTx4943 | TGTGCTTACAGTGACTGAGA | 1502 |
| | nt | | TCT | |
| Guide | | | | |
| polynucleotide | | SEQ ID | | SEQ ID |
| name | Target site sequence⁹ | NO | Spacer sequence | NO |
| gRNA2055 | CTCAGATGTCTATGTGTTTG | 1503 | CUCAGAUGUCUAUGUGUU | 1523 |
| | GG | | U | |
| gRNA1193 | CCTCAGATGTCTATGTGTTT | 1504 | CCUCAGAUGUCUAUGUGU | 1524 |
| | GGG | | UU | |
| gRNA2057 | CCCTCAGATGTCTATGTGTT | 1505 | CCCUCAGAUGUCUAUGUG | 1525 |
| | TGGG | | UUU | |
| gRNA2058 | TCCCTCAGATGTCTATGTGT | 1506 | UCCCUCAGAUGUCUAUGU | 1526 |
| | TTGGG | | GUUU | |
| gRNA2059 | ATCCCTCAGATGTCTATGTG | 1507 | AUCCCUCAGAUGUCUAUG | 1527 |
| | TTTGGG | | UGUUU | |
| gRNA2060 | CTCACCTCTGCAAGTATTGG | 1508 | CUCACCUCUGCAAGUAUU | 1528 |
| | GG | | G | |
| gRNA1202 | TCTCACCTCTGCAAGTATTG | 1509 | UCUCACCUCUGCAAGUAU | 1529 |
| | GGG | | UG | |
| gRNA2062 | CTCTCACCTCTGCAAGTATT | 1510 | CUCUCACCUCUGCAAGUA | 1530 |
| | GGGG | | UUG | |
| gRNA2063 | TCTCTCACCTCTGCAAGTAT | 1511 | UCUCUCACCUCUGCAAGU | 1531 |
| | TGGGG | | AUUG | |
| gRNA2064 | TTCTCTCACCTCTGCAAGTA | 1512 | UUCUCUCACCUCUGCAAG | 1532 |
| | TTGGGG | | UAUUG | |
| gRNA2065 | CTTACAATGACTGAGATCTT | 1513 | CUUACAAUGACUGAGAUC | 1533 |
| | GG | | U | |
| gRNA1204 | GCTTACAATGACTGAGATCT | 1514 | GCUUACAAUGACUGAGAU | 1534 |
| | TGG | | CU | |
| gRNA2067 | TGCTTACAATGACTGAGATC | 1515 | UGCUUACAAUGACUGAGA | 1535 |
| | TTGG | | UCU | |
| gRNA2068 | GTGCTTACAATGACTGAGAT | 1516 | GUGCUUACAAUGACUGAG | 1536 |
| | CTTGG | | AUCU | |
| gRNA2069 | TGTGCTTACAATGACTGAGA | 1517 | UGUGCUUACAAUGACUGA | 1537 |
| | TCTTGG | | GAUCU | |
| gRNA2070 | CTTACAGTGACTGAGATCTT | 1518 | CUUACAGUGACUGAGAUC | 1538 |
| | GG | | U | |
| gRNA1999 | GCTTACAGTGACTGAGATCT | 1519 | GCUUACAGUGACUGAGAU | 1539 |
| | TGG | | CU | |
| gRNA2072 | TGCTTACAGTGACTGAGATC | 1520 | UGCUUACAGUGACUGAGA | 1540 |
| | TTGG | | UCU | |
| gRNA2073 | GTGCTTACAGTGACTGAGAT | 1521 | GUGCUUACAGUGACUGAG | 1541 |
| | CTTGG | | AUCU | |
| gRNA2074 | TGTGCTTACAGTGACTGAGA | 1522 | UGUGCUUACAGUGACUGA | 1542 |
| | TCTTGG | | GAUCU | |

⁹PAM sequences shown as underlined plain text; target nucleotides are in bold; human sequence nucleotides complementary to a primate (cyno) target sequence but not to a human target sequence are in bold underline.

TABLE 2B
Representative spacer and target site sequences relating to introduction of a
missense mutation to a complement factor B polynucleotide using base editing.

| | | | Associated | | |
|---|---|---|---|---|---|
| | | Cas | missense | | |
| Guide polynucleotide name | Editor | variant | mutations | | |
| GuideDesign14_ABE_NGA_20nt_3- | ABE_NGA_20nt_ | spCas9 | S699P | | |
| 9_005_−_31951540_spCas9 | 3-9_005 | VRQR | | | |
| | | ABE | | | |
| GuideDesign14_ABE_NGG_20nt_3- | ABE_NGG_20nt_ | spCas9 | S699P | | |
| 9_002_−_31951541_spCas9 | 3-9_002 | ABE | | | |
| GuideDesign14_ABE_NNNRRT_21nt_5- | ABE_NNNRRT_21 | saCas9 | V177A | | |
| 14_014_−_31947370_saCas9 | nt_5-14_014 | KKH ABE | | | |
| GuideDesign14_ABE_NNNRRT_21nt_5- | ABE_NNNRRT_21 | saCas9 | V177A | | |
| 14_014_−_31947376_saCas9 | nt_5-14_014 | KKH ABE | | | |
| GuideDesign14_ABE_NNNRRT_21nt_5- | ABE_NNNRRT_21 | saCas9 | S278P | | |
| 14_014_−_31947998_saCas9 | nt_5-14_014 | KKH ABE | | | |
| GuideDesign14_ABE_NNNRRT_21nt_5- | ABE_NNNRRT_21 | saCas9 | H526R | | |
| 14_014_+_31950345_saCas9 | nt_5-14_014 | KKH ABE | | | |
| GuideDesign14_CBE_NGA_20nt_4- | CBE_NGA_20nt_ | spCas9 | A525V, H526Y | | |
| 9_006_+_31950348_spCas9 | 4-9_006 | VRQR | | | |
| | | CBE | | | |
| GuideDesign14_CBE_NGA_20nt_4- | CBE_NGA_20nt_ | spCas9 | H526Y | | |
| 9_006_+_31950351_spCas9 | 4-9_006 | VRQR | | | |
| | | CBE | | | |
| GuideDesign14_CBE_NGC_20nt_4- | CBE_NGC_20nt_ | spCas9 | R202Q, R203Q | | |
| 9_009_−_31947453_spCas9 | 4-9_009 | NGC CBE | | | |
| GuideDesign14_CBE_NGC_20nt_4- | CBE_NGC_20nt_ | spCas9 | S280N | | |
| 9_009_−_31948005_spCas9 | 4-9_009 | NGC CBE | | | |
| GuideDesign14_CBE_NGC_20nt_4- | CBE_NGC_20nt_ | spCas9 | S278L | | |
| 9_009_+_31948009_spCas9 | 4-9_009 | NGC CBE | | | |
| GuideDesign14_CBE_NGC_20nt_4- | CBE_NGC_20nt_ | spCas9 | S278L | | |
| 9_009_+_31948013_spCas9 | 4-9_009 | NGC CBE | | | |
| GuideDesign14_CBE_NGC_20nt_4- | CBE_NGC_20nt_ | spCas9 | T353I | | |
| 9_009_+_31948843_spCas9 | 4-9_009 | NGC CBE | | | |
| GuideDesign14_CBE_NGG_20nt_4- | CBE_NGG_20nt_ | spCas9 | R175K, V177M | | |
| 9_003_−_31947372_spCas9 | 4-9_003 | CBE | | | |
| GuideDesign14_CBE_NGG_20nt_4- | CBE_NGG_20nt_ | spCas9 | V177M | | |
| 9_003_−_31947373_spCas9 | 4-9_003 | CBE | | | |
| GuideDesign14_CBE_NGG_20nt_4- | CBE_NGG_20nt_ | spCas9 | V177I | | |
| 9_003_−_31947374_spCas9 | 4-9_003 | CBE | | | |
| GuideDesign14_CBE_NGG_20nt_4- | CBE_NGG_20nt_ | spCas9 | T353I | | |
| 9_003_+_31948842_spCas9 | 4-9_003 | CBE | | | |
| GuideDesign14_CBE_NGG_20nt_4- | CBE_NGG_20nt_ | spCas9 | A525V, H526Y | | |
| 9_003_+_31950347_spCas9 | 4-9_003 | CBE | | | |
| GuideDesign14_CBE_NNGRRT_21nt_3- | CBE_NNGRRT_21 | saCas9 | R175K, V177I, G1 | | |
| 12_012_−_31947370_saCas9 | nt_3-12_012 | CBE | 78S | | |
| GuideDesign14_CBE_NNGRRT_21nt_3- | CBE_NNGRRT_21 | saCas9 | A524V, A525V, H5 | | |
| 12_012_+_31950345_saCas9 | nt_3-12_012 | CBE | 26Y | | |
| GuideDesign14_CBE_NNNRRT_21nt_3- | CBE_NNNRRT_21 | saCas9 | R175K, V1771, G1 | | |
| 12_015_−_31947370_saCas9 | nt_3-12_015 | KKH CBE | 78S | | |
| GuideDesign14_CBE_NNNRRT_21nt_3- | CBE_NNNRRT_21 | saCas9 | V177I, G178N, S17 | | |
| 12_015_−_31947376_saCas9 | nt_3-12_015 | KKH CBE | 9N | | |
| GuideDesign14_CBE_NNNRRT_21nt_3- | CBE_NNNRRT_21 | saCas9 | R202Q, R203Q | | |
| 12_015_−_31947448_saCas9 | nt_3-12_015 | KKH CBE | | | |
| GuideDesign14_CBE_NNNRRT_21nt_3- | CBE_NNNRRT_21 | saCas9 | R259Q | | |
| 12_015_−_31947938_saCas9 | nt_3-12_015 | KKH CBE | | | |
| GuideDesign14_CBE_NNNRRT_21nt_3- | CBE_NNNRRT_21 | saCas9 | G277E, D279N, S2 | | |
| 12_015_−_31947998_saCas9 | nt_3-12_015 | KKH CBE | 80N | | |
| GuideDesign14_CBE_NNNRRT_21nt_3- | CBE_NNNRRT_21 | saCas9 | D574N, D576N, V5 | | |
| 12_015_−_31950698_saCas9 | nt_3-12_015 | KKH CBE | 77I | | |
| GuideDesign14_CBE_NNNRRT_21nt_3- | CBE_NNNRRT_21 | saCas9 | A524V, A525V, H5 | | |
| 12_015_+_31950345_saCas9 | nt_3-12_015 | KKH CBE | 26Y | | |
| GuideDesign14_CBE_NNNRRT_21nt_3- | CBE_NNNRRT_21 | saCas9 | S699F | | |
| 12_015_+_31951549_saCas9 | nt_3-12_015 | KKH CBE | | | |
| GuideDesign15_ABE_NGA_20nt_3- | ABE_NGA_20nt_ | spCas9 | E255G | | |
| 9_005_+_31947939_spCas9 | 3-9_005 | VRQR | | | |
| | | ABE | | | |
| GuideDesign15_ABE_NGA_20nt_3- | ABE_NGA_20nt_ | spCas9 | E255G, Q256R | | |
| 9_005_+_31947942_spCas9 | 3-9_005 | VRQR | | | |
| | | ABE | | | |
| GuideDesign15_ABE_NGA_20nt_3- | ABE_NGA_20nt_ | spCas9 | K258G | | |
| 9_005_+_31947952_spCas9 | 3-9_005 | VRQR | | | |
| | | ABE | | | |
| GuideDesign15_ABE_NGA_20nt_3- | ABE_NGA_20nt_ | spCas9 | D470G, E471G | | |
| 9_005_+_31950044_spCas9 | 3-9_005 | VRQR | | | |
| | | ABE | | | |
| GuideDesign15_ABE_NGG_20nt_3- | ABE_NGG_20nt_ | spCas9 | Q257R, K258G | | |
| 9_002_+_31947951_spCas9 | 3-9_002 | ABE | | | |
| GuideDesign15_ABE_NGG_20nt_3- | ABE_NGG_20nt_ | spCas9 | D276G | | |
| 9_002_+_31948008_spCas9 | 3-9_002 | ABE | | | |
| GuideDesign15_ABE_NNGRRT_21nt_5- | ABE_NNGRRT_21 | saCas9 | D470G, E471G, S4 | | |
| 14_011_+_31950042_saCas9 | nt_5-14_011 | ABE | 72G | | |
| GuideDesign15_ABE_NNNRRT_21nt_5- | ABE_NNNRRT_21 | saCas9 | E255G, Q256R | | |
| 14_014_+_31947939_saCas9 | nt_5-14_014 | KKH ABE | | | |
| GuideDesign15_ABE_NNNRRT_21nt_5- | ABE_NNNRRT_21 | saCas9 | D470G, E471G, S4 | | |
| 14_014_+_31950042_saCas9 | nt_5-14_014 | KKH ABE | 72G | | |
| GuideDesign15_CBE_NGA_20nt_4- | CBE_NGA_20nt_ | spCas9 | G254E, E255K | | |
| 9_006_−_31947929_spCas9 | 4-9_006 | VRQR | | | |
| | | CBE | | | |
| GuideDesign15_CBE_NGA_20nt_4- | CBE_NGA_20nt_ | spCas9 | D276N, G277K | | |
| 9_006_−_31947995_spCas9 | 4-9_006 | VRQR | | | |
| | | CBE | | | |
| GuideDesign15_CBE_NGA_20nt_4- | CBE_NGA_20nt_ | spCas9 | D389N | | |
| 9_006_−_31948940_spCas9 | 4-9_006 | VRQR | | | |
| | | CBE | | | |
| GuideDesign15_CBE_NGA_20nt_4- | CBE_NGA_20nt_ | spCas9 | D389N, G390R | | |
| 9_006_−_31948943_spCas9 | 4-9_006 | VRQR | | | |
| | | CBE | | | |
| GuideDesign15_CBE_NGA_20nt_4- | CBE_NGA_20nt_ | spCas9 | E471K | | |
| 9_006_−_31950033_spCas9 | 4-9_006 | VRQR | | | |
| | | CBE | | | |
| GuideDesign15_CBE_NGA_20nt_4- | CBE_NGA_20nt_ | spCas9 | G697D, D698N | | |
| 9_006_−_31951538_spCas9 | 4-9_006 | VRQR | | | |
| | | CBE | | | |
| GuideDesign15_CBE_NGA_20nt_4- | CBE_NGA_20nt_ | spCas9 | G697D, D698N | | |
| 9_006_−_31951540_spCas9 | 4-9_006 | VRQR | | | |
| | | CBE | | | |
| GuideDesign15_CBE_NGC_20nt_4- | CBE_NGC_20nt_ | spCas9 | G697D | | |
| 9_009_−_31951535_spCas9 | 4-9_009 | NGC CBE | | | |
| GuideDesign15_CBE_NGG_20nt_4- | CBE_NGG_20nt_ | spCas9 | R696K, G697S | | |
| 9_003_−_31951456_spCas9 | 4-9_003 | CBE | | | |
| GuideDesign15_CBE_NGG_20nt_4- | CBE_NGG_20nt_ | spCas9 | G697S | | |
| 9_003_−_31951457_spCas9 | 4-9_003 | CBE | | | |
| GuideDesign15_CBE_NGG_20nt_4- | CBE_NGG_20nt_ | spCas9 | G697S | | |
| 9_003_−_31951458_spCas9 | 4-9_003 | CBE | | | |
| GuideDesign15_CBE_NNGRRT_21nt_3- | CBE_NNGRRT_21 | saCas9 | G254E, E255K | | |
| 12_012_−_31947927_saCas9 | nt_3-12_012 | CBE | | | |
| GuideDesign15_CBE_NNGRRT_21nt_3- | CBE_NNGRRT_21 | saCas9 | E471K, S472N | | |
| 12_012_−_31950031_saCas9 | nt_3-12_012 | CBE | | | |
| GuideDesign15_CBE_NNGRRT_21nt_3- | CBE_NNGRRT_21 | saCas9 | R696K, G697S | | |
| 12_012_−_31951455_saCas9 | nt_3-12_012 | CBE | | | |
| GuideDesign15_CBE_NNNRRT_21nt_3- | CBE_NNNRRT_21 | saCas9 | A231T, E232K, A2 | | |
| 12_015_−_31947758_saCas9 | nt_3-12_015 | KKH CBE | 33T | | |
| GuideDesign15_CBE_NNNRRT_21nt_3- | CBE_NNNRRT_21 | saCas9 | G254E, E255K | | |
| 12_015_−_31947927_saCas9 | nt_3-12_015 | KKH CBE | | | |
| GuideDesign15_CBE_NNNRRT_21nt_3- | CBE_NNNRRT_21 | saCas9 | D276N, G277K, D2 | | |
| 12_015_−_31947994_saCas9 | nt_3-12_015 | KKH CBE | 79N | | |
| GuideDesign15_CBE_NNNRRT_21nt_3- | CBE_NNNRRT_21 | saCas9 | D389N, G390R | | |
| 12_015_−_31948942_saCas9 | nt_3-12_015 | KKH CBE | | | |
| GuideDesign15_CBE_NNNRRT_21nt_3- | CBE_NNNRRT_21 | saCas9 | E471K, S472N | | |
| 12_015_−_31950031_saCas9 | nt_3-12_015 | KKH CBE | | | |
| GuideDesign15_CBE_NNNRRT_21nt_3- | CBE_NNNRRT_21 | saCas9 | R696K, G697S | | |
| 12_015_−_31951455_saCas9 | nt_3-12_015 | KKH CBE | | | |
| | | SEQ | | SEQ | |
| Guide polynucleotide | | ID | | ID | PAM |
| name | Target sequence | NO | Spacer sequence | NO | sequence |
| GuideDesign14_ABE_N | CAGAATCACCTGCAAGG | 1543 | CAGAAUCACCUG | 1600 | GGA |
| GA_20nt_3-9_005_−_ | AGAGGA | | CAAGGAGA | | |
| 31951540_spCas9 | | | | | |
| GuideDesign14_ABE_N | CCAGAATCACCTGCAAG | 1544 | CCAGAAUCACCU | 1601 | AGG |
| GG_20nt_3-9_002_−_ | GAGAGG | | GCAAGGAG | | |
| 31951541_spCas9 | | | | | |
| GuideDesign14_ABE_N | GCCCACCTTCCTTGTGC | 1545 | GCCCACCUUCCU | 1602 | GGGG |
| NNRRT_21nt_5- | CAATGGGGAT | | UGUGCCAAU | | AT |
| 14_014_−_ | | | | | |
| 31947370_saCas9 | | | | | |
| GuideDesign14_ABE_N | CTGGCTGCCCACCTTCC | 1546 | CUGGCUGCCCAC | 1603 | GCCA |
| NNRRT_21nt_5- | TTGTGCCAAT | | CUUCCUUGU | | AT |
| 14_014_−_ | | | | | |
| 31947376_saCas9 | | | | | |
| GuideDesign14_ABE_N | TGCTGTCTGATCCATCT | 1547 | UGCUGUCUGAUC | 1604 | CCAG |
| NNRRT_21nt_5- | AGCACCAGGT | | CAUCUAGCA | | GT |
| 14_014_−_ | | | | | |
| 31947998_saCas9 | | | | | |
| GuideDesign14_ABE_N | ACAGCAGCACATTGTTT | 1548 | ACAGCAGCACAU | 1605 | GTGG |
| NNRRT_21nt_5- | CACTGTGGAT | | UGUUUCACU | | AT |
| 14_014_+_31950345_sa | | | | | |
| Cas9 | | | | | |
| GuideDesign14_CBE_N | GCAGCACATTGTTTCAC | 1549 | GCAGCACAUUGU | 1606 | GGA |
| GA_20nt_4- | TGTGGA | | UUCACUGU | | |
| 9_006_+_31950348_spC | | | | | |
| as9 | | | | | |
| GuideDesign14_CBE_N | GCACATTGTTTCACTGT | 1550 | GCACAUUGUUUC | 1607 | TGA |
| GA_20nt_4- | GGATGA | | ACUGUGGA | | |
| 9_006_+_31950351_spC | | | | | |
| as9 | | | | | |
| GuideDesign14_CBE_N | ACGTTCGCCGCTGGGAG | 1551 | ACGUUCGCCGCU | 1608 | CGC |
| GC_20nt_4-9_009_- | CCACGC | | GGGAGCCA | | |
| 31947453_spCas9 | | | | | |
| GuideDesign14_CBE_N | CAATGCTGTCTGATCCA | 1552 | CAAUGCUGUCUG | 1609 | AGC |
| GC_20nt_4-9_009_−_ | TCTAGC | | AUCCAUCU | | |
| 31948005_spCas9 | | | | | |
| GuideDesign14_CBE_N | GATGGATCAGACAGCAT | 1553 | GAUGGAUCAGAC | 1610 | GGC |
| GC_20nt_4- | TGGGGC | | AGCAUUGG | | |
| 9_009_+_31948009_spC | | | | | |
| as9 | | | | | |
| GuideDesign14_CBE_N | GATCAGACAGCATTGGG | 1554 | GAUCAGACAGCA | 1611 | AGC |
| GC_20nt_4- | GCCAGC | | UUGGGGCC | | |
| 9_009_+_31948013_spC | | | | | |
| as9 | | | | | |
| GuideDesign14_CBE_N | TCAGGGACTAACACCAA | 1555 | UCAGGGACUAAC | 1612 | GGC |
| GC_20nt_4- | GAAGGC | | ACCAAGAA | | |
| 9_009_+_31948843_spC | | | | | |
| as9 | | | | | |
| GuideDesign14_CBE_N | CCACCTTCCTTGTGCCA | 1556 | CCACCUUCCUUG | 1613 | GGG |
| GG_20nt_4-9_003_−_ | ATGGGG | | UGCCAAUG | | |
| 31947372_spCas9 | | | | | |
| GuideDesign14_CBE_N | CCCACCTTCCTTGTGCC | 1557 | CCCACCUUCCUU | 1614 | GGG |
| GG_20nt_4-9_003_−_ | AATGGG | | GUGCCAAU | | |
| 31947373_spCas9 | | | | | |
| GuideDesign14_CBE_N | GCCCACCTTCCTTGTGC | 1558 | GCCCACCUUCCU | 1615 | TGG |
| GG_20nt_4-9_003_−_ | CAATGG | | UGUGCCAA | | |
| 31947374_spCas9 | | | | | |
| GuideDesign14_CBE_N | GTCAGGGACTAACACCA | 1559 | GUCAGGGACUAA | 1616 | AGG |
| GG_20nt_4- | AGAAGG | | CACCAAGA | | |
| 9_003_+_31948842_spC | | | | | |
| as9 | | | | | |
| GuideDesign14_CBE_N | AGCAGCACATTGTTTCA | 1560 | AGCAGCACAUUG | 1617 | TGG |
| GG_20nt_4- | CTGTGG | | UUUCACUG | | |
| 9_003_+_31950347_spC | | | | | |
| as9 | | | | | |
| GuideDesign14_CBE_N | GCCCACCTTCCTTGTGC | 1561 | GCCCACCUUCCU | 1618 | GGGG |
| NGRRT_21nt_3- | CAATGGGGAT | | UGUGCCAAU | | AT |
| 12_012_−_ | | | | | |
| 31947370_saCas9 | | | | | |
| GuideDesign14_CBE_N | ACAGCAGCACATTGTTT | 1562 | ACAGCAGCACAU | 1619 | GTGG |
| NGRRT_21nt_3- | CACTGTGGAT | | UGUUUCACU | | AT |
| 12_012_+_31950345_sa | | | | | |
| Cas9 | | | | | |
| GuideDesign14_CBE_N | GCCCACCTTCCTTGTGC | 1563 | GCCCACCUUCCU | 1620 | GGGG |
| NNRRT_21nt_3- | CAATGGGGAT | | UGUGCCAAU | | AT |
| 12_015_−_ | | | | | |
| 31947370_saCas9 | | | | | |
| GuideDesign14_CBE_N | CTGGCTGCCCACCTTCC | 1564 | CUGGCUGCCCAC | 1621 | GCCA |
| NNRRT_21nt_3- | TTGTGCCAAT | | CUUCCUUGU | | AT |
| 12_015_−_ | | | | | |
| 31947376_saCas9 | | | | | |
| GuideDesign14_CBE_N | CGTTCGCCGCTGGGAGC | 1565 | CGUUCGCCGCUG | 1622 | CAGG |
| NNRRT_21nt_3- | CACGCAGGGT | | GGAGCCACG | | GT |
| 12_015_−_ | | | | | |
| 31947448_saCas9 | | | | | |
| GuideDesign14_CBE_N | TCTTCCGCTTCTGTTGT | 1566 | UCUUCCGCUUCU | 1623 | CTGG |
| NNRRT_21nt_3- | TCCCCTGGGT | | GUUGUUCCC | | GT |
| 12_015_−_ | | | | | |
| 31947938_saCas9 | | | | | |
| GuideDesign14_CBE_N | TGCTGTCTGATCCATCT | 1567 | UGCUGUCUGAUC | 1624 | CCAG |
| NNRRT_21nt_3- | AGCACCAGGT | | CAUCUAGCA | | GT |
| 12_015_−_ | | | | | |
| 31947998_saCas9 | | | | | |
| GuideDesign14_CBE_N | AACGTCATAGTCATAAA | 1568 | AACGUCAUAGUC | 1625 | AGGA |
| NNRRT_21nt_3- | ATTCAGGAAT | | AUAAAAUUC | | AT |
| 12_015_−_ | | | | | |
| 31950698_saCas9 | | | | | |
| GuideDesign14_CBE_N | ACAGCAGCACATTGTTT | 1569 | ACAGCAGCACAU | 1626 | GTGG |
| NNRRT_21nt_3- | CACTGTGGAT | | UGUUUCACU | | AT |
| 12_015_+_31950345_sa | | | | | |
| Cas9 | | | | | |
| GuideDesign14_CBE_N | TGCAGGTGATTCTGGCG | 1570 | UGCAGGUGAUUC | 1627 | CTTG |
| NNRRT_21nt_3- | GCCCCTTGAT | | UGGCGGCCC | | AT |
| 12_015_+_31951549_sa | | | | | |
| Cas9 | | | | | |
| GuideDesign15_ABE_N | CCCAGGGGAACAACAGA | 1571 | CCCAGGGGAACA | 1628 | GGA |
| GA_20nt_3- | AGCGGA | | ACAGAAGC | | |
| 9_005_+_31947939_spC | | | | | |
| as9 | | | | | |
| GuideDesign15_ABE_N | AGGGGAACAACAGAAGC | 1572 | AGGGGAACAACA | 1629 | AGA |
| GA_20nt_3- | GGAAGA | | GAAGCGGA | | |
| 9_005_+_31947942_spC | | | | | |
| as9 | | | | | |
| GuideDesign15_ABE_N | CAGAAGCGGAAGATCGT | 1573 | CAGAAGCGGAAG | 1630 | GGA |
| GA_20nt_3- | CCTGGA | | AUCGUCCU | | |
| 9_005_+_31947952_spC | | | | | |
| as9 | | | | | |
| GuideDesign15_ABE_N | GCCAGATGAAAGCCAGT | 1574 | GCCAGAUGAAAG | 1631 | TGA |
| GA_20nt_3- | CTCTGA | | CCAGUCUC | | |
| 9_005_+_31950044_spC | | | | | |
| as9 | | | | | |
| GuideDesign15_ABE_N | ACAGAAGCGGAAGATCG | 1575 | ACAGAAGCGGAA | 1632 | TGG |
| GG_20nt_3- | TCCTGG | | GAUCGUCC | | |
| 9_002_+_31947951_spC | | | | | |
| as9 | | | | | |
| GuideDesign15_ABE_N | AGATGGATCAGACAGCA | 1576 | AGAUGGAUCAGA | 1633 | GGG |
| GG_20nt_3- | TTGGGG | | CAGCAUUG | | |
| 9_002_+_31948008_spC | | | | | |
| as9 | | | | | |
| GuideDesign15_ABE_N | CTGCCAGATGAAAGCCA | 1577 | CUGCCAGAUGAA | 1634 | CTGA |
| NGRRT_21nt_5- | GTCTCTGAGT | | AGCCAGUCU | | GT |
| 14_011_+_31950042_sa | | | | | |
| Cas9 | | | | | |
| GuideDesign15_ABE_N | CCCAGGGGAACAACAGA | 1578 | CCCAGGGGAACA | 1635 | GAAG |
| NNRRT_21nt_5- | AGCGGAAGAT | | ACAGAAGCG | | AT |
| 14_014_+_31947939_sa | | | | | |
| Cas9 | | | | | |
| GuideDesign15_ABE_N | CTGCCAGATGAAAGCCA | 1579 | CUGCCAGAUGAA | 1636 | CTGA |
| NNRRT_21nt_5- | GTCTCTGAGT | | AGCCAGUCU | | GT |
| 14_014_+_31950042_sa | | | | | |
| Cas9 | | | | | |
| GuideDesign15_CBE_N | TTGTTCCCCTGGGTGCC | 1580 | UUGUUCCCCUGG | 1637 | AGA |
| GA_20nt_4-9_006_−_ | AGGAGA | | GUGCCAGG | | |
| 31947929_spCas9 | | | | | |
| GuideDesign15_CBE_N | TGATCCATCTAGCACCA | 1581 | UGAUCCAUCUAG | 1638 | AGA |
| GA_20nt_4-9_006_−_ | GGTAGA | | CACCAGGU | | |
| 31947995_spCas9 | | | | | |
| GuideDesign15_CBE_N | ACCATCAGTCATGAGGA | 1582 | ACCAUCAGUCAU | 1639 | TGA |
| GA_20nt_4-9_006_−_ | TGATGA | | GAGGAUGA | | |
| 31948940_spCas9 | | | | | |
| GuideDesign15_CBE_N | CTGACCATCAGTCATGA | 1583 | CUGACCAUCAGU | 1640 | TGA |
| GA_20nt_4-9_006_−_ | GGATGA | | CAUGAGGA | | |
| 31948943_spCas9 | | | | | |
| GuideDesign15_CBE_N | CTTTCATCTGGCAGAGA | 1584 | CUUUCAUCUGGC | 1641 | AGA |
| GA_20nt_4-9_006_−_ | AGGAGA | | AGAGAAGG | | |
| 31950033_spCas9 | | | | | |
| GuideDesign15_CBE_N | GAATCACCTGCAAGGAG | 1585 | GAAUCACCUGCA | 1642 | AGA |
| GA_20nt_4-9_006_−_ | AGGAGA | | AGGAGAGG | | |
| 31951538_spCas9 | | | | | |
| GuideDesign15_CBE_N | CAGAATCACCTGCAAGG | 1586 | CAGAAUCACCUG | 1643 | GGA |
| GA_20nt_4-9_006_−_ | AGAGGA | | CAAGGAGA | | |
| 31951540_spCas9 | | | | | |
| GuideDesign15_CBE_N | TCACCTGCAAGGAGAGG | 1587 | UCACCUGCAAGG | 1644 | AGC |
| GC_20nt_4-9_009_−_ | AGAAGC | | AGAGGAGA | | |
| 31951535_spCas9 | | | | | |
| GuideDesign15_CBE_N | TCTCACCTCTGCAAGTA | 1588 | UCUCACCUCUGC | 1645 | GGG |
| GG_20nt_4-9_003_−_ | TTGGGG | | AAGUAUUG | | |
| 31951456_spCas9 | | | | | |
| GuideDesign15_CBE_N | CTCTCACCTCTGCAAGT | 1589 | CUCUCACCUCUG | 1646 | GGG |
| GG_20nt_4-9_003_−_ | ATTGGG | | CAAGUAUU | | |
| 31951457_spCas9 | | | | | |
| GuideDesign15_CBE_N | TCTCTCACCTCTGCAAG | 1590 | UCUCUCACCUCU | 1647 | TGG |
| GG_20nt_4-9_003_−_ | TATTGG | | GCAAGUAU | | |
| 31951458_spCas9 | | | | | |
| GuideDesign15_CBE_N | TGTTGTTCCCCTGGGTG | 1591 | UGUUGUUCCCCU | 1648 | GAGA |
| NGRRT_21nt_3- | CCAGGAGAGT | | GGGUGCCAG | | GT |
| 12_012_−_ | | | | | |
| 31947927_saCas9 | | | | | |
| GuideDesign15_CBE_N | GGCTTTCATCTGGCAGA | 1592 | GGCUUUCAUCUG | 1649 | GAGA |
| NGRRT_21nt_3- | GAAGGAGAAT | | GCAGAGAAG | | AT |
| 12_012_−_ | | | | | |
| 31950031_saCas9 | | | | | |
| GuideDesign15_CBE_N | TTCTCTCACCTCTGCAA | 1593 | UUCUCUCACCUC | 1650 | TGGG |
| NGRRT_21nt_3- | GTATTGGGGT | | UGCAAGUAU | | GT |
| 12_012_−_ | | | | | |
| 31951455_saCas9 | | | | | |
| GuideDesign15_CBE_N | GAAAGCTTCGGCCACCT | 1594 | GAAAGCUUCGGC | 1651 | AGGG |
| NNRRT_21nt_3- | CTTGAGGGGT | | CACCUCUUG | | GT |
| 12_015_−_ | | | | | |
| 31947758_saCas9 | | | | | |
| GuideDesign15_CBE_N | TGTTGTTCCCCTGGGTG | 1595 | UGUUGUUCCCCU | 1652 | GAGA |
| NNRRT_21nt_3- | CCAGGAGAGT | | GGGUGCCAG | | GT |
| 12_015_−_ | | | | | |
| 31947927_saCas9 | | | | | |
| GuideDesign15_CBE_N | GTCTGATCCATCTAGCA | 1596 | GUCUGAUCCAUC | 1653 | GTAG |
| NNRRT_21nt_3- | CCAGGTAGAT | | UAGCACCAG | | AT |
| 12_015_−_ | | | | | |
| 31947994_saCas9 | | | | | |
| GuideDesign15_CBE_N | CTTCTGACCATCAGTCA | 1597 | CUUCUGACCAUC | 1654 | GATG |
| NNRRT_21nt_3- | TGAGGATGAT | | AGUCAUGAG | | AT |
| 12_015_−_ | | | | | |
| 31948942_saCas9 | | | | | |
| GuideDesign15_CBE_N | GGCTTTCATCTGGCAGA | 1598 | GGCUUUCAUCUG | 1655 | GAGA |
| NNRRT_21nt_3- | GAAGGAGAAT | | GCAGAGAAG | | AT |
| 12_015_−_ | | | | | |
| 31950031_saCas9 | | | | | |
| GuideDesign15_CBE_N | TTCTCTCACCTCTGCAA | 1599 | UUCUCUCACCUC | 1656 | TGGG |
| NNRRT_21nt_3- | GTATTGGGGT | | UGCAAGUAU | | GT |
| 12_015_−_ | | | | | |
| 31951455_saCas9 | | | | | |

TABLE 2C
Representative spacer and target site sequences relating to introduction of a
missense mutation to a complement factor B polynucleotide using base editing.

| Guide | | | | | |
|---|---|---|---|---|---|
| polynucleotide | | | Associate missense | | |
| name | Editor | Cas variant | mutations | | |
| gRNA1517 | ABE_NGG_20nt_3-9_002 | spCas9 ABE | T353A | | |
| gRNA1518 | ABE_NGG_20nt_3-9_002 | spCas9 ABE | H526R | | |
| gRNA1519 | ABE_NGG_20nt_3-9_002 | spCas9 ABE | S278P | | |
| gRNA1520 | ABE_NGG_20nt_3-9_002 | spCas9 ABE | V177A | | |
| gRNA1521 | ABE_NGG_20nt_3-9_002 | spCas9 ABE | V177A | | |
| gRNA1522 | ABE_NGG_20nt_3-9_002 | spCas9 ABE | V177A | | |
| gRNA1523 | CBE_NGG_20nt_4-9_003 | spCas9 CBE | P171S | | |
| gRNA1524 | CBE_NGG_20nt_4-9_003 | spCas9 CBE | P171F | | |
| gRNA1525 | CBE_NGG_20nt_4-9_003 | spCas9 CBE | R259W | | |
| gRNA1526 | CBE_NGG_20nt_4-9_003 | spCas9 CBE | S278L | | |
| gRNA1527 | CBE_NGG_20nt_4-9_003 | spCas9 CBE | D576N | | |
| gRNA1528 | CBE_NGG_20nt_4-9_003 | spCas9 CBE | R259Q | | |
| gRNA1529 | ABE_NGA_20nt_3-9_005 | spCas9 | H526R | | |
| | | VRQR ABE | | | |
| gRNA1530 | ABE_NGA_20nt_3-9_005 | spCas9 | H526R | | |
| | | VRQR ABE | | | |
| gRNA1531 | ABE_NGA_20nt_3-9_005 | spCas9 | S699P | | |
| | | VRQR ABE | | | |
| gRNA1532 | ABE_NGA_20nt_3-9_005 | spCas9 | S699P | | |
| | | VRQR ABE | | | |
| gRNA1533 | ABE_NGA_20nt_3-9_005 | spCas9 | S278P | | |
| | | VRQR ABE | | | |
| gRNA1534 | CBE_NGA_20nt_4-9_006 | spCas9 | P171F | | |
| | | VRQR CBE | | | |
| gRNA1535 | CBE_NGA_20nt_4-9_006 | spCas9 | R259W | | |
| | | VRQR CBE | | | |
| gRNA1536 | CBE_NGA_20nt_4-9_006 | spCas9 | S699F | | |
| | | VRQR CBE | | | |
| gRNA1537 | CBE_NGA_20nt_4-9_006 | spCas9 | D576N | | |
| | | VRQR CBE | | | |
| gRNA1538 | ABE_NGC_20nt_3-9_008 | spCas9 NGC | D279G, S280G | | |
| | | ABE | | | |
| gRNA1539 | ABE_NGC_20nt_3-9_008 | spCas9 NGC | T353A | | |
| | | ABE | | | |
| gRNA1540 | ABE_NGC_20nt_3-9_008 | spCas9 NGC | Y575C, D576G | | |
| | | ABE | | | |
| gRNA1541 | ABE_NNGRRT_21nt_5- | saCas9 ABE | H526R | | |
| | 14_011 | | | | |
| gRNA1542 | ABE_NNGRRT_21nt_5- | saCas9 ABE | V177A | | |
| | 14_011 | | | | |
| gRNA1543 | CBE_NNGRRT_21nt_3- | saCas9 CBE | D574N, D576N, V577I | | |
| | 12_012 | | | | |
| gRNA1544 | CBE_NNGRRT_21nt_3- | saCas9 CBE | R259Q | | |
| | 12_012 | | | | |
| gRNA1545 | CBE_NNGRRT_21nt_3- | saCas9 CBE | R202Q, R203Q | | |
| | 12_012 | | | | |
| gRNA1546 | ABE_NNNRRT_21nt_5- | saCas9 KKH | L275P, S278P | | |
| | 14_014 | ABE | | | |
| gRNA1547 | CBE_NNNRRT_21nt_3- | saCas9 KKH | P171F | | |
| | 12_015 | CBE | | | |
| gRNA1548 | CBE_NNNRRT_21nt_3- | saCas9 KKH | S699F | | |
| | 12_015 | CBE | | | |
| gRNA1549 | ABE_NGG_20nt_3-9_002 | spCas9 ABE | D276G | | |
| gRNA1550 | ABE_NGG_20nt_3-9_002 | spCas9 ABE | D276G | | |
| gRNA1551 | CBE_NGG_20nt_4-9_003 | spCas9 CBE | E471K, S472N | | |
| gRNA1552 | CBE_NGG_20nt_4-9_003 | spCas9 CBE | E255K | | |
| gRNA1553 | CBE_NGG_20nt_4-9_003 | spCas9 CBE | E232K, A233T | | |
| gRNA1554 | CBE_NGG_20nt_4-9_003 | spCas9 CBE | E232K, A233T | | |
| gRNA1555 | CBE_NGG_20nt_4-9_003 | spCas9 CBE | A231T, E232K | | |
| gRNA1556 | ABE_NGA_20nt_3-9_005 | spCas9 | E232G | | |
| | | VRQR ABE | | | |
| gRNA1557 | CBE_NGA_20nt_4-9_006 | spCas9 | E471K | | |
| | | VRQR CBE | | | |
| gRNA1558 | CBE_NGA_20nt_4-9_006 | spCas9 | E255K | | |
| | | VRQR CBE | | | |
| gRNA1559 | CBE_NNGRRT_21nt_3- | saCas9 CBE | A231T, E232K, A233T | | |
| | 12_012 | | | | |
| Guide | Target sequence | SEQ | | SEQ | |
| polynucleotide | (bold text indicates | ID | | ID | PAM |
| name | a target nucleotide) | NO | Spacer sequence | NO | sequence |
| gRNA1517 | GTCAGGGACTAACACCAAGA | 1657 | GUCAGGGACUAACA | 1700 | AGG |
| | AGG | | CCAAGA | | |
| gRNA1518 | AGCAGCACATTGTTTCACTG | 1658 | AGCAGCACAUUGUU | 1701 | TGG |
| | TGG | | UCACUG | | |
| gRNA1519 | TGTCTGATCCATCTAGCACC | 1659 | UGUCUGAUCCAUCU | 1702 | AGG |
| | AGG | | AGCACC | | |
| gRNA1520 | GCCCACCTTCCTTGTGCCAA | 1660 | GCCCACCUUCCUUG | 1703 | TGG |
| | TGG | | UGCCAA | | |
| gRNA1521 | CCCACCTTCCTTGTGCCAAT | 1661 | CCCACCUUCCUUGU | 1704 | GGG |
| | GGG | | GCCAAU | | |
| gRNA1522 | CCACCTTCCTTGTGCCAATG | 1662 | CCACCUUCCUUGUG | 1705 | GGG |
| | GGG | | CCAAUG | | |
| gRNA1523 | CGGGCATCCCCATTGGCACA | 1663 | CGGGCAUCCCCAUU | 1706 | AGG |
| | AGG | | GGCACA | | |
| gRNA1524 | CATCCCCATTGGCACAAGGA | 1664 | CAUCCCCAUUGGCA | 1707 | AGG |
| | AGG | | CAAGGA | | |
| gRNA1525 | ACAGAAGCGGAAGATCGTCC | 1665 | ACAGAAGCGGAAGA | 1708 | TGG |
| | TGG | | UCGUCC | | |
| gRNA1526 | AGATGGATCAGACAGCATTG | 1666 | AGAUGGAUCAGACA | 1709 | GGG |
| | GGG | | GCAUUG | | |
| gRNA1527 | ACGTCATAGTCATAAAATTC | 1667 | ACGUCAUAGUCAUA | 1710 | AGG |
| | AGG | | AAAUUC | | |
| gRNA1528 | TTCCGCTTCTGTTGTTCCCC | 1668 | UUCCGCUUCUGUUG | 1711 | TGG |
| | TGG | | UUCCCC | | |
| gRNA1529 | GCAGCACATTGTTTCACTGT | 1669 | GCAGCACAUUGUUU | 1712 | GGA |
| | GGA | | CACUGU | | |
| gRNA1530 | GCACATTGTTTCACTGTGGA | 1670 | GCACAUUGUUUCAC | 1713 | TGA |
| | TGA | | UGUGGA | | |
| gRNA1531 | GCCGCCAGAATCACCTGCAA | 1671 | GCCGCCAGAAUCAC | 1714 | GGA |
| | GGA | | CUGCAA | | |
| gRNA1532 | CGCCAGAATCACCTGCAAGG | 1672 | CGCCAGAAUCACCU | 1715 | AGA |
| | AGA | | GCAAGG | | |
| gRNA1533 | TGATCCATCTAGCACCAGGT | 1673 | UGAUCCAUCUAGCA | 1716 | AGA |
| | AGA | | CCAGGU | | |
| gRNA1534 | GGGCATCCCCATTGGCACAA | 1674 | GGGCAUCCCCAUUG | 1717 | GGA |
| | GGA | | GCACAA | | |
| gRNA1535 | CAGAAGCGGAAGATCGTCCT | 1675 | CAGAAGCGGAAGAU | 1718 | GGA |
| | GGA | | CGUCCU | | |
| gRNA1536 | AGGTGATTCTGGCGGCCCCT | 1676 | AGGUGAUUCUGGCG | 1719 | TGA |
| | TGA | | GCCCCU | | |
| gRNA1537 | CGTCATAGTCATAAAATTCA | 1677 | CGUCAUAGUCAUAA | 1720 | GGA |
| | GGA | | AAUUCA | | |
| gRNA1538 | GATCAGACAGCATTGGGGCC | 1678 | GAUCAGACAGCAUU | 1721 | AGC |
| | AGC | | GGGGCC | | |
| gRNA1539 | TCAGGGACTAACACCAAGAA | 1679 | UCAGGGACUAACAC | 1722 | GGC |
| | GGC | | CAAGAA | | |
| gRNA1540 | CTATGACGTTGCCCTGATCA | 1680 | CUAUGACGUUGCCC | 1723 | AGC |
| | AGC | | UGAUCA | | |
| gRNA1541 | ACAGCAGCACATTGTTTCAC | 1681 | ACAGCAGCACAUUG | 1724 | GTGGAT |
| | TGTGGAT | | UUUCACU | | |
| gRNA1542 | GCCCACCTTCCTTGTGCCAA | 1682 | GCCCACCUUCCUUG | 1725 | GGGGAT |
| | TGGGGAT | | UGCCAAU | | |
| gRNA1543 | AACGTCATAGTCATAAAATT | 1683 | AACGUCAUAGUCAU | 1726 | AGGAAT |
| | CAGGAAT | | AAAAUUC | | |
| gRNA1544 | TCTTCCGCTTCTGTTGTTCC | 1684 | UCUUCCGCUUCUGU | 1727 | CTGGGT |
| | CCTGGGT | | UGUUCCC | | |
| gRNA1545 | CGTTCGCCGCTGGGAGCCAC | 1685 | CGUUCGCCGCUGGG | 1728 | CAGGGT |
| | GCAGGGT | | AGCCACG | | |
| gRNA1546 | GTCTGATCCATCTAGCACCA | 1686 | GUCUGAUCCAUCUA | 1729 | GTAGAT |
| | GGTAGAT | | GCACCAG | | |
| gRNA1547 | GGGCATCCCCATTGGCACAA | 1687 | GGGCAUCCCCAUUG | 1730 | GAAGGT |
| | GGAAGGT | | GCACAAG | | |
| gRNA1548 | AGGTGATTCTGGCGGCCCCT | 1688 | AGGUGAUUCUGGCG | 1731 | GATAGT |
| | TGATAGT | | GCCCCUU | | |
| gRNA1549 | CTAGATGGATCAGACAGCAT | 1689 | CUAGAUGGAUCAGA | 1732 | TGG |
| | TGG | | CAGCAU | | |
| gRNA1550 | TAGATGGATCAGACAGCATT | 1690 | UAGAUGGAUCAGAC | 1733 | GGG |
| | GGG | | AGCAUU | | |
| gRNA1551 | TGGCTTTCATCTGGCAGAGA | 1691 | UGGCUUUCAUCUGG | 1734 | AGG |
| | AGG | | CAGAGA | | |
| gRNA1552 | CTGTTGTTCCCCTGGGTGCC | 1692 | CUGUUGUUCCCCUG | 1735 | AGG |
| | AGG | | GGUGCC | | |
| gRNA1553 | AAAGCTTCGGCCACCTCTTG | 1693 | AAAGCUUCGGCCAC | 1736 | AGG |
| | AGG | | CUCUUG | | |
| gRNA1554 | AAGCTTCGGCCACCTCTTGA | 1694 | AAGCUUCGGCCACC | 1737 | GGG |
| | GGG | | UCUUGA | | |
| gRNA1555 | AGCTTCGGCCACCTCTTGAG | 1695 | AGCUUCGGCCACCU | 1738 | GGG |
| | GGG | | CUUGAG | | |
| gRNA1556 | CGAAGCTTTCCTGTCTTCCC | 1696 | CGAAGCUUUCCUGU | 1739 | TGA |
| | TGA | | CUUCCC | | |
| gRNA1557 | GGCTTTCATCTGGCAGAGAA | 1697 | GGCUUUCAUCUGGC | 1740 | GGA |
| | GGA | | AGAGAA | | |
| gRNA1558 | TGTTGTTCCCCTGGGTGCCA | 1698 | UGUUGUUCCCCUGG | 1741 | GGA |
| | GGA | | GUGCCA | | |
| gRNA1559 | GAAAGCTTCGGCCACCTCTT | 1699 | GAAAGCUUCGGCCA | 1742 | AGGGGT |
| | GAGGGGT | | CCUCUUG | | |

TABLE 2D
Representative spacer and target site sequences relating to disrupting of a
complement factor B splice site or introducing a new stop codon into a complement
factor B polynucleotide using base editing.

| Guide | Target | | SEQ | | SEQ | |
|---|---|---|---|---|---|---|
| polynucleotide | site | | ID | | ID | |
| name | name | Target site | NO | Spacer sequence | NO | Editor |
| gRNA1181 | TSBTx | CTGCAGGATTGCAC | 1743 | CUGCAGGAUUGCA | 1899 | ABE_NGC_20 |
| | 3814 | AACATG | | CAACAUG | | nt_3-9_008 |
| gRNA1182 | TSBTx | ATAGGCCCATTTGT | 1744 | AUAGGCCCAUUUG | 1900 | ABE_NGC_20 |
| | 3815 | CTCCCC | | UCUCCCC | | nt_3-9_008 |
| gRNA1183 | TSBTx | CTCTGCAGGTTGGT | 1745 | CUCUGCAGGUUGG | 1901 | ABE_NGC_20 |
| | 3816 | GTAATC | | UGUAAUC | | nt_3-9_008 |
| gRNA1184 | TSBTx | ACGCTCTCACCTGA | 1746 | ACGCUCUCACCUG | 1902 | ABE_NGC_20 |
| | 3817 | TAGTCT | | AUAGUCU | | nt_3-9_008 |
| gRNA1185 | TSBTx | CTTACAATGACTGA | 1747 | CUUACAAUGACUG | 1903 | ABE_NGC_20 |
| | 3818 | GATCTT | | AGAUCUU | | nt_3-9_008 |
| gRNA1186 | TSBTx | TCACCTCCGTTGTC | 1748 | UCACCUCCGUUGU | 1904 | ABE_NGC_20 |
| | 3819 | ACAGAT | | CACAGAU | | nt_3-9_008 |
| gRNA1187 | TSBTx | TCGCTTACCTCCAG | 1749 | UCGCUUACCUCCA | 1905 | ABE_NGC_20 |
| | 3820 | ACAAGA | | GACAAGA | | nt_3-9_008 |
| gRNA1188 | TSBTx | CACCCACAGGTGGC | 1750 | CACCCACAGGUGG | 1906 | ABE_NGG_20 |
| | 3821 | AAGTTA | | CAAGUUA | | nt_3-9_002 |
| gRNA1189 | TSBTx | ACAGACCACAAGTT | 1751 | ACAGACCACAAGU | 1907 | ABE_NGG_20 |
| | 3822 | GAAGTC | | UGAAGUC | | nt_3-9_002 |
| gRNA1190 | TSBTx | TCTGCAGGATTGCA | 1752 | UCUGCAGGAUUGC | 1908 | ABE_NGG_20 |
| | 3823 | CAACAT | | ACAACAU | | nt_3-9_002 |
| gRNA1191 | TSBTx | GCAGGATTGCACAA | 1753 | GCAGGAUUGCACA | 1909 | ABE_NGG_20 |
| | 3824 | CATGGG | | ACAUGGG | | nt_3-9_002 |
| gRNA1192 | TSBTx | CCCTCAGATGTCTA | 1754 | CCCUCAGAUGUCU | 1910 | ABE_NGG_20 |
| | 3825 | TGTGTT | | AUGUGUU | | nt_3-9_002 |
| gRNA1193 | TSBTx | CCTCAGATGTCTAT | 1755 | CCUCAGAUGUCUA | 1911 | ABE_NGG_20 |
| | 3826 | GTGTTT | | UGUGUUU | | nt_3-9_002 |
| gRNA1194 | TSBTx | CTCAGATGTCTATG | 1756 | CUCAGAUGUCUAU | 1912 | ABE_NGG_20 |
| | 3827 | TGTTTG | | GUGUUUG | | nt_3-9_002 |
| gRNA1195 | TSBTx | GTGTCCCCAGCGCC | 1757 | GUGUCCCCAGCGC | 1913 | ABE_NGG_20 |
| | 3828 | CTTCAA | | CCUUCAA | | nt_3-9_002 |
| gRNA1196 | TSBTx | TGTCCCCAGCGCCC | 1758 | UGUCCCCAGCGCC | 1914 | ABE_NGG_20 |
| | 3829 | TTCAAA | | CUUCAAA | | nt_3-9_002 |
| gRNA1197 | TSBTx | CTACTTCAGGAGGG | 1759 | CUACUUCAGGAGG | 1915 | ABE_NGG_20 |
| | 3830 | GAGAAG | | GGAGAAG | | nt_3-9_002 |
| gRNA1198 | TSBTx | TACTTCAGGAGGGG | 1760 | UACUUCAGGAGGG | 1916 | ABE_NGG_20 |
| | 3831 | AGAAGC | | GAGAAGC | | nt_3-9_002 |
| gRNA1199 | TSBTx | CTCCTTGCAGGTGA | 1761 | CUCCUUGCAGGUG | 1917 | ABE_NGG_20 |
| | 3832 | TTCTGG | | AUUCUGG | | nt_3-9_002 |
| gRNA1200 | TSBTx | TGCAGGTTGGTGTA | 1762 | UGCAGGUUGGUGU | 1918 | ABE_NGG_20 |
| | 3833 | ATCAGC | | AAUCAGC | | nt_3-9_002 |
| gRNA1201 | TSBTx | GCAGGTTGGTGTAA | 1763 | GCAGGUUGGUGUA | 1919 | ABE_NGG_20 |
| | 3834 | TCAGCT | | AUCAGCU | | nt_3-9_002 |
| gRNA1202 | TSBTx | TCTCACCTCTGCAA | 1764 | UCUCACCUCUGCA | 1920 | ABE_NGG_20 |
| | 3835 | GTATTG | | AGUAUUG | | nt_3-9_002 |
| gRNA1203 | TSBTx | CTTACTTTGTTGCT | 1765 | CUUACUUUGUUGC | 1921 | ABE_NGG_20 |
| | 3836 | GGCAAG | | UGGCAAG | | nt_3-9_002 |
| gRNA1204 | TSBTx | GCTTACAATGACTG | 1766 | GCUUACAAUGACU | 1922 | ABE_NGG_20 |
| | 3837 | AGATCT | | GAGAUCU | | nt_3-9_002 |
| gRNA1205 | TSBTx | TCACCCAGATAATC | 1767 | UCACCCAGAUAAU | 1923 | ABE_NGG_20 |
| | 3838 | CTCCCT | | CCUCCCU | | nt_3-9_002 |
| gRNA1206 | TSBTx | CCTTCTGACCATCA | 1768 | CCUUCUGACCAUC | 1924 | ABE_NGG_20 |
| | 3839 | GTCATG | | AGUCAUG | | nt_3-9_002 |
| gRNA1207 | TSBTx | CAAAGGTCACCTTG | 1769 | CAAAGGUCACCUU | 1925 | ABE_NGG_20 |
| | 3840 | GCAGGA | | GGCAGGA | | nt_3-9_002 |
| gRNA1208 | TSBTx | CTCGCTTACCTCCA | 1770 | CUCGCUUACCUCC | 1926 | ABE_NGG_20 |
| | 3841 | GACAAG | | AGACAAG | | nt_3-9_002 |
| gRNA1209 | TSBTx | TCAAAGCAATCCAC | 1771 | UCAAAGCAAUCCA | 1927 | ABE_NGA_20 |
| | 3842 | TGTCCA | | CUGUCCA | | nt_3-9_005 |
| gRNA1210 | TSBTx | CTCCCAGACTCCTT | 1772 | CUCCCAGACUCCU | 1928 | ABE_NGA_20 |
| | 3843 | CATGTA | | UCAUGUA | | nt_3-9_005 |
| gRNA1211 | TSBTx | CCCAGGGGAACAAC | 1773 | CCCAGGGGAACAA | 1929 | ABE_NGA_20 |
| | 3844 | AGAAGC | | CAGAAGC | | nt_3-9_005 |
| gRNA1212 | TSBTx | ACAGGTGGCAAGTT | 1774 | ACAGGUGGCAAGU | 1930 | ABE_NGA_20 |
| | 3845 | ATGGTG | | UAUGGUG | | nt_3-9_005 |
| gRNA1213 | TSBTx | GCCAGATGAAAGCC | 1775 | GCCAGAUGAAAGC | 1931 | ABE_NGA_20 |
| | 3846 | AGTCTC | | CAGUCUC | | nt_3-9_005 |
| gRNA1214 | TSBTx | GTCCCCAGCGCCCT | 1776 | GUCCCCAGCGCCC | 1932 | ABE_NGA_20 |
| | 3847 | TCAAAG | | UUCAAAG | | nt_3-9_005 |
| gRNA1215 | TSBTx | TACAGAAAGGCAGC | 1777 | UACAGAAAGGCAG | 1933 | ABE_NGA_20 |
| | 3848 | TGTGAG | | CUGUGAG | | nt_3-9_005 |
| gRNA1216 | TSBTx | GAGGACTCACTTGA | 1778 | GAGGACUCACUUG | 1934 | ABE_NGA_20 |
| | 3849 | ATGAAA | | AAUGAAA | | nt_3-9_005 |
| gRNA1217 | TSBTx | TCTCACCTTATCCC | 1779 | UCUCACCUUAUCC | 1935 | ABE_NGA_20 |
| | 3850 | CATTCT | | CCAUUCU | | nt_3-9_005 |
| gRNA1218 | TSBTx | ATCCTTACCTACGC | 1780 | AUCCUUACCUACG | 1936 | ABE_NGA_20 |
| | 3851 | TGACCT | | CUGACCU | | nt_3-9_005 |
| gRNA1219 | TSBTx | TTACCTACGCTGAC | 1781 | UUACCUACGCUGA | 1937 | ABE_NGA_20 |
| | 3852 | CTTGAT | | CCUUGAU | | nt_3-9_005 |
| gRNA1220 | TSBTx | CTCCCTACCGATCA | 1782 | CUCCCUACCGAUC | 1938 | ABE_NGA_20 |
| | 3853 | TTTGGT | | AUUUGGU | | nt_3-9_005 |
| gRNA1221 | TSBTx | CTGACCATCAGTCA | 1783 | CUGACCAUCAGUC | 1939 | ABE_NGA_20 |
| | 3854 | TGAGGA | | AUGAGGA | | nt_3-9_005 |
| gRNA1222 | TSBTx | CTTCTCACCTCCGT | 1784 | CUUCUCACCUCCG | 1940 | ABE_NGA_20 |
| | 3855 | TGTCAC | | UUGUCAC | | nt_3-9_005 |
| gRNA1223 | TSBTx | TCCCTCAGATGTCT | 1785 | UCCCUCAGAUGUC | 1941 | ABE_NNGRR |
| | 3856 | ATGTGTT | | UAUGUGUU | | T_21nt_5- |
| | | | | | | 14_011 |
| gRNA1224 | TSBTx | CTGCCAGATGAAAG | 1786 | CUGCCAGAUGAAA | 1942 | ABE_NNGRR |
| | 3857 | CCAGTCT | | GCCAGUCU | | T_21nt_5- |
| | | | | | | 14_011 |
| gRNA1225 | TSBTx | TTCTCTCACCTCTG | 1787 | UUCUCUCACCUCU | 1943 | ABE_NNGRR |
| | 3858 | CAAGTAT | | GCAAGUAU | | T_21nt_5- |
| | | | | | | 14_011 |
| gRNA1226 | TSBTx | CCTTACCTACGCTG | 1788 | CCUUACCUACGCU | 1944 | ABE_NNGRR |
| | 3859 | ACCTTGA | | GACCUUGA | | T_21nt_5- |
| | | | | | | 14_011 |
| gRNA1227 | TSBTx | ACTCACCCAGATAA | 1789 | ACUCACCCAGAUA | 1945 | ABE_NNGRR |
| | 3860 | TCCTCCC | | AUCCUCCC | | T_21nt_5- |
| | | | | | | 14_011 |
| gRNA1228 | TSBTx | TCCCTTCTGACCAT | 1790 | UCCCUUCUGACCA | 1946 | ABE_NNGRR |
| | 3861 | CAGTCAT | | UCAGUCAU | | T_21nt_5- |
| | | | | | | 14_011 |
| gRNA1229 | TSBTx | CTTCAGGTGTGACC | 1791 | CUUCAGGUGUGAC | 1947 | ABE_NNNRR |
| | 3862 | ACCACTC | | CACCACUC | | T_21nt_5- |
| | | | | | | 14_014 |
| gRNA1230 | TSBTx | CCCTCTCCACAGAC | 1792 | CCCUCUCCACAGA | 1948 | ABE_NNNRR |
| | 3863 | CACAAGT | | CCACAAGU | | T_21nt_5- |
| | | | | | | 14_014 |
| gRNA1231 | TSBTx | CTACAGAAAGGCAG | 1793 | CUACAGAAAGGCA | 1949 | ABE_NNNRR |
| | 3864 | CTGTGAG | | GCUGUGAG | | T_21nt_5- |
| | | | | | | 14_014 |
| gRNA1232 | TSBTx | GTATGTCTTACTTT | 1794 | GUAUGUCUUACUU | 1950 | ABE_NNNRR |
| | 3865 | GTTGCTG | | UGUUGCUG | | T_21nt_5- |
| | | | | | | 14_014 |
| gRNA1233 | TSBTx | TGTCTTACTTTGTT | 1795 | UGUCUUACUUUGU | 1951 | ABE_NNNRR |
| | 3866 | GCTGGCA | | UGCUGGCA | | T_21nt_5- |
| | | | | | | 14_014 |
| gRNA1234 | TSBTx | TGCATCCTTACCTA | 1796 | UGCAUCCUUACCU | 1952 | ABE_NNNRR |
| | 3867 | CGCTGAC | | ACGCUGAC | | T_21nt_5- |
| | | | | | | 14_014 |
| gRNA1235 | TSBTx | GGATTCTGTGCTTA | 1797 | GGAUUCUGUGCUU | 1953 | ABE_NNNRR |
| | 3868 | CAATGAC | | ACAAUGAC | | T_21nt_5- |
| | | | | | | 14_014 |
| gRNA1236 | TSBTx | CTTGTATCTCCCTA | 1798 | CUUGUAUCUCCCU | 1954 | ABE_NNNRR |
| | 3869 | CCGATCA | | ACCGAUCA | | T_21nt_5- |
| | | | | | | 14_014 |
| gRNA1237 | TSBTx | CTTCTGACCATCAG | 1799 | CUUCUGACCAUCA | 1955 | ABE_NNNRR |
| | 3870 | TCATGAG | | GUCAUGAG | | T_21nt_5- |
| | | | | | | 14_014 |
| gRNA1238 | TSBTx | CCCTAACCTCTGAC | 1800 | CCCUAACCUCUGA | 1956 | ABE_NNNRR |
| | 3871 | CTTCATA | | CCUUCAUA | | T_21nt_5- |
| | | | | | | 14_014 |
| gRNA1239 | TSBTx | AGGAGGATTCCACC | 1801 | AGGAGGAUUCCAC | 1957 | ABE_NNNRR |
| | 3872 | TTCTCAA | | CUUCUCAA | | T_21nt_5- |
| | | | | | | 14_014 |
| gRNA1240 | TSBTx | ATGCTTCTCACCTC | 1802 | AUGCUUCUCACCU | 1958 | ABE_NNNRR |
| | 3873 | CGTTGTC | | CCGUUGUC | | T_21nt_5- |
| | | | | | | 14_014 |
| gRNA1241 | TSBTx | TTCTCCAAGAGGGC | 1803 | UUCUCCAAGAGGG | 1959 | CBE_NNGRRT |
| | 3874 | CAGGCAC | | CCAGGCAC | | 21nt_3- |
| | | | | | | 12_012 |
| gRNA1242 | TSBTx | ACCAAAAGACTGTC | 1804 | ACCAAAAGACUGU | 1960 | CBE_NNGRRT |
| | 3875 | AGGAAGG | | CAGGAAGG | | 21nt_3- |
| | | | | | | 12_012 |
| gRNA1243 | TSBTx | ACCTGCCAAGTGAA | 1805 | ACCUGCCAAGUGA | 1961 | CBE_NNGRRT |
| | 3876 | TGGCCGA | | AUGGCCGA | | 21nt_3- |
| | | | | | | 12_012 |
| gRNA1244 | TSBTx | GAGCAACATGTGTT | 1806 | GAGCAACAUGUGU | 1962 | CBE_NNGRRT |
| | 3877 | CAAAGTC | | UCAAAGUC | | 21nt_3- |
| | | | | | | 12_012 |
| gRNA1245 | TSBTx | TACACCAACCTGCA | 1807 | UACACCAACCUGC | 1963 | CBE_NNGRRT |
| | 3878 | GAGGCAG | | AGAGGCAG | | 21nt_3- |
| | | | | | | 12_012 |
| gRNA1246 | TSBTx | GGCTTTCATCTGGC | 1808 | GGCUUUCAUCUGG | 1964 | CBE_NNGRRT |
| | 3879 | AGAGAAG | | CAGAGAAG | | 21nt_3- |
| | | | | | | 12_012 |
| gRNA1247 | TSBTx | GACATCTGAGGGAT | 1809 | GACAUCUGAGGGA | 1965 | CBE_NNGRRT |
| | 3880 | AAAAAGG | | UAAAAAGG | | 21nt_3- |
| | | | | | | 12_012 |
| gRNA1248 | TSBTx | TGTTGTTCCCCTGG | 1810 | UGUUGUUCCCCUG | 1966 | CBE_NNGRRT |
| | 3881 | GTGCCAG | | GGUGCCAG | | 21nt_3- |
| | | | | | | 12_012 |
| gRNA1249 | TSBTx | ATCGCACCTGCCAA | 1811 | AUCGCACCUGCCA | 1967 | CBE_NNNRRT |
| | 3882 | GTGAATG | | AGUGAAUG | | 21nt_3- |
| | | | | | | 12_015 |
| gRNA1250 | TSBTx | TGGCCGATGGAGTG | 1812 | UGGCCGAUGGAGU | 1968 | CBE_NNNRRT |
| | 3883 | GGCAGAC | | GGGCAGAC | | 21nt_3- |
| | | | | | | 12_015 |
| gRNA1251 | TSBTx | TCCCAGCGGCGAAC | 1813 | UCCCAGCGGCGAA | 1969 | CBE_NNNRRT |
| | 3884 | GTGTCAG | | CGUGUCAG | | 21nt_3- |
| | | | | | | 12_015 |
| gRNA1252 | TSBTx | CCCAGGGGAACAAC | 1814 | CCCAGGGGAACAA | 1970 | CBE_NNNRRT |
| | 3885 | AGAAGCG | | CAGAAGCG | | 21nt_3- |
| | | | | | | 12_015 |
| gRNA1253 | TSBTx | GTCACGAAGCAGCT | 1815 | GUCACGAAGCAGC | 1971 | CBE_NNNRRT |
| | 3886 | CAATGAA | | UCAAUGAA | | 21nt_3- |
| | | | | | | 12_015 |
| gRNA1254 | TSBTx | GGCCCTCCAGGCAG | 1816 | GGCCCUCCAGGCA | 1972 | CBE_NNNRRT |
| | 3887 | TGTACAG | | GUGUACAG | | 21nt_3- |
| | | | | | | 12_015 |
| gRNA1255 | TSBTx | TTTCTACCAAATGA | 1817 | UUUCUACCAAAUG | 1973 | CBE_NNNRRT |
| | 3888 | TCGGTAG | | AUCGGUAG | | 21nt_3- |
| | | | | | | 12_015 |
| gRNA1256 | TSBTx | GCAACCATGGCAGG | 1818 | GCAACCAUGGCAG | 1974 | CBE_NNNRRT |
| | 3889 | CCAAGAT | | GCCAAGAU | | 21nt_3- |
| | | | | | | 12_015 |
| gRNA1257 | TSBTx | TCAGCCAGGGCAGC | 1819 | UCAGCCAGGGCAG | 1975 | CBE_NNNRRT |
| | 3890 | ACTTGAA | | CACUUGAA | | 21nt_3- |
| | | | | | | 12_015 |
| gRNA1258 | TSBTx | CTTACTTTGTTGCT | 1820 | CUUACUUUGUUGC | 1976 | CBE_NNNRRT |
| | 3891 | GGCAAGT | | UGGCAAGU | | 21nt_3- |
| | | | | | | 12_015 |
| gRNA1259 | TSBTx | CTCCCCTCCTGAAG | 1821 | CUCCCCUCCUGAA | 1977 | CBE_NNNRRT |
| | 3892 | TAGGAGA | | GUAGGAGA | | 21nt_3- |
| | | | | | | 12_015 |
| gRNA1260 | TSBTx | TTTGAAGGGCGCTG | 1822 | UUUGAAGGGCGCU | 1978 | CBE_NNNRRT |
| | 3893 | GGGACAC | | GGGGACAC | | 21nt_3- |
| | | | | | | 12_015 |
| gRNA1261 | TSBTx | TCTTGGCCTGCCAT | 1823 | UCUUGGCCUGCCA | 1979 | CBE_NNNRRT |
| | 3894 | GGTTGCT | | UGGUUGCU | | 21nt_3- |
| | | | | | | 12_015 |
| gRNA1262 | TSBTx | TGGCCTGCCATGGT | 1824 | UGGCCUGCCAUGG | 1980 | CBE_NNNRRT |
| | 3895 | TGCTTGT | | UUGCUUGU | | 21nt_3- |
| | | | | | | 12_015 |
| gRNA1263 | TSBTx | ATGAAGGAGTCTGG | 1825 | AUGAAGGAGUCUG | 1981 | CBE_NNNRRT |
| | 3896 | GAGAGTC | | GGAGAGUC | | 21nt_3- |
| | | | | | | 12_015 |
| gRNA1264 | TSBTx | GGAGTCTGGGAGAG | 1826 | GGAGUCUGGGAGA | 1982 | CBE_NNNRRT |
| | 3897 | TCAGAAA | | GUCAGAAA | | 21nt_3- |
| | | | | | | 12_015 |
| gRNA1265 | TSBTx | CGCTGCAGAGGTAT | 1827 | CGCUGCAGAGGUA | 1983 | CBE_NNNRRT |
| | 3898 | GAGACAT | | UGAGACAU | | 21nt_3- |
| | | | | | | 12_015 |
| gRNA1266 | TSBTx | ACTCCATCGGCCAT | 1828 | ACUCCAUCGGCCA | 1984 | CBE_NNNRRT |
| | 3899 | TCACTTG | | UUCACUUG | | 21nt_3- |
| | | | | | | 12_015 |
| gRNA1267 | TSBTx | GGGGCCAGTATTCC | 1829 | GGGGCCAGUAUUC | 1985 | CBE_NNNRRT |
| | 3900 | CCGTTCT | | CCCGUUCU | | 21nt_3- |
| | | | | | | 12_015 |
| gRNA1268 | TSBTx | GGATTGCTTTGAGA | 1830 | GGAUUGCUUUGAG | 1986 | CBE_NNNRRT |
| | 3901 | AGGGGGG | | AAGGGGGG | | 21nt_3- |
| | | | | | | 12_015 |
| gRNA1269 | TSBTx | TTCAGGGTGCTCCA | 1831 | UUCAGGGUGCUCC | 1987 | CBE_NNNRRT |
| | 3902 | GGACCCC | | AGGACCCC | | 21nt_3- |
| | | | | | | 12_015 |
| gRNA1270 | TSBTx | GCTCCAGGACCCCG | 1832 | GCUCCAGGACCCC | 1988 | CBE_NNNRRT |
| | 3903 | TAGATCT | | GUAGAUCU | | 21nt_3- |
| | | | | | | 12_015 |
| gRNA1271 | TSBTx | TGGTCACACCTGAA | 1833 | UGGUCACACCUGA | 1989 | CBE_NNNRRT |
| | 3943 | GAGAAAG | | AGAGAAAG | | 21nt_3- |
| | | | | | | 12_015 |
| gRNA1272 | TSBTx | CTTACCTCCAGACA | 1834 | CUUACCUCCAGAC | 1990 | CBE_NNNRRT |
| | 3944 | AGAGGCC | | AAGAGGCC | | 21nt_3- |
| | | | | | | 12_015 |
| gRNA1356 | TSBTx | CTCCTTCCGACTTC | 1835 | CUCCUUCCGACUU | 1991 | CBE_NGG_20 |
| | 3997 | TCCAAG | | CUCCAAG | | nt_4-9_003 |
| gRNA1357 | TSBTx | TCCTTCCGACTTCT | 1836 | UCCUUCCGACUUC | 1992 | CBE_NGG_20 |
| | 3998 | CCAAGA | | UCCAAGA | | nt_4-9_003 |
| gRNA1358 | TSBTx | TCTCCAAGAGGGCC | 1837 | UCUCCAAGAGGGC | 1993 | CBE_NGG_20 |
| | 3999 | AGGCAC | | CAGGCAC | | nt_4-9_003 |
| gRNA1359 | TSBTx | CTCAAGACCAAAAG | 1838 | CUCAAGACCAAAA | 1994 | CBE_NGG_20 |
| | 4000 | ACTGTC | | GACUGUC | | nt_4-9_003 |
| gRNA1360 | TSBTx | AGACCAAAAGACTG | 1839 | AGACCAAAAGACU | 1995 | CBE_NGG_20 |
| | 4001 | TCAGGA | | GUCAGGA | | nt_4-9_003 |
| gRNA1361 | TSBTx | CCTGCCAAGTGAAT | 1840 | CCUGCCAAGUGAA | 1996 | CBE_NGG_20 |
| | 4002 | GGCCGA | | UGGCCGA | | nt_4-9_003 |
| gRNA1362 | TSBTx | CTCCCAGCGGCGAA | 1841 | CUCCCAGCGGCGA | 1997 | CBE_NGG_20 |
| | 4003 | CGTGTC | | ACGUGUC | | nt_4-9_003 |
| gRNA1363 | TSBTx | CAGCGGCGAACGTG | 1842 | CAGCGGCGAACGU | 1998 | CBE_NGG_20 |
| | 4004 | TCAGGA | | GUCAGGA | | nt_4-9_003 |
| gRNA1364 | TSBTx | CGGCGAACGTGTCA | 1843 | CGGCGAACGUGUC | 1999 | CBE_NGG_20 |
| | 4005 | GGAAGG | | AGGAAGG | | nt_4-9_003 |
| gRNA1365 | TSBTx | CGTGTCAGGAAGGT | 1844 | CGUGUCAGGAAGG | 2000 | CBE_NGG_20 |
| | 4006 | GGCTCT | | UGGCUCU | | nt_4-9_003 |
| gRNA1366 | TSBTx | TTTTCTACCAAATG | 1845 | UUUUCUACCAAAU | 2001 | CBE_NGG_20 |
| | 4007 | ATCGGT | | GAUCGGU | | nt_4-9_003 |
| gRNA1367 | TSBTx | TTTCTACCAAATGA | 1846 | UUUCUACCAAAUG | 2002 | CBE_NGG_20 |
| | 4008 | TCGGTA | | AUCGGUA | | nt_4-9_003 |
| gRNA1368 | TSBTx | GCAACAAAGTAAGA | 1847 | GCAACAAAGUAAG | 2003 | CBE_NGG_20 |
| | 4009 | CATACT | | ACAUACU | | nt_4-9_003 |
| gRNA1369 | TSBTx | ACCTCTTTCAAGTG | 1848 | ACCUCUUUCAAGU | 2004 | CBE_NGG_20 |
| | 4010 | CTGCCC | | GCUGCCC | | nt_4-9_003 |
| gRNA1370 | TSBTx | GAAACTCCAAGATG | 1849 | GAAACUCCAAGAU | 2005 | CBE_NGG_20 |
| | 4011 | AGGATT | | GAGGAUU | | nt_4-9_003 |
| gRNA1371 | TSBTx | AAACTCCAAGATGA | 1850 | AAACUCCAAGAUG | 2006 | CBE_NGG_20 |
| | 4012 | GGATTT | | AGGAUUU | | nt_4-9_003 |
| gRNA1372 | TSBTx | CACCAACCTGCAGA | 1851 | CACCAACCUGCAG | 2007 | CBE_NGG_20 |
| | 4013 | GGCAGT | | AGGCAGU | | nt_4-9_003 |
| gRNA1373 | TSBTx | ACCAACCTGCAGAG | 1852 | ACCAACCUGCAGA | 2008 | CBE_NGG_20 |
| | 4014 | GCAGTG | | GGCAGUG | | nt_4-9_003 |
| gRNA1374 | TSBTx | TGCCTTTCTGTAGG | 1853 | UGCCUUUCUGUAG | 2009 | CBE_NGG_20 |
| | 4015 | GGAGGT | | GGGAGGU | | nt_4-9_003 |
| gRNA1375 | TSBTx | AAATGGGCCTATAA | 1854 | AAAUGGGCCUAUA | 2010 | CBE_NGG_20 |
| | 4016 | AGGACA | | AAGGACA | | nt_4-9_003 |
| gRNA1376 | TSBTx | CCCTCCTGAAGTAG | 1855 | CCCUCCUGAAGUA | 2011 | CBE_NGG_20 |
| | 4017 | GAGAGT | | GGAGAGU | | nt_4-9_003 |
| gRNA1377 | TSBTx | TGGCCTGCCATGGT | 1856 | UGGCCUGCCAUGG | 2012 | CBE_NGG_20 |
| | 4018 | TGCTTG | | UUGCUUG | | nt_4-9_003 |
| gRNA1378 | TSBTx | TAGACATCTGAGGG | 1857 | UAGACAUCUGAGG | 2013 | CBE_NGG_20 |
| | 4019 | ATAAAA | | GAUAAAA | | nt_4-9_003 |
| gRNA1379 | TSBTx | CATCTGAGGGATAA | 1858 | CAUCUGAGGGAUA | 2014 | CBE_NGG_20 |
| | 4020 | AAAGGA | | AAAAGGA | | nt_4-9_003 |
| gRNA1380 | TSBTx | TGCAATCCTGCAGA | 1859 | UGCAAUCCUGCAG | 2015 | CBE_NGG_20 |
| | 4021 | AGAGAC | | AAGAGAC | | nt_4-9_003 |
| gRNA1381 | TSBTx | GTGCGGTTCCAGCC | 1860 | GUGCGGUUCCAGC | 2016 | CBE_NGG_20 |
| | 4022 | TTCAGG | | CUUCAGG | | nt_4-9_003 |
| gRNA1382 | TSBTx | TGCGGTTCCAGCCT | 1861 | UGCGGUUCCAGCC | 2017 | CBE_NGG_20 |
| | 4023 | TCAGGA | | UUCAGGA | | nt_4-9_003 |
| gRNA1383 | TSBTx | GACCCAAATTTTGG | 1862 | GACCCAAAUUUUG | 2018 | CBE_NGG_20 |
| | 4024 | GGTATG | | GGGUAUG | | nt_4-9_003 |
| gRNA1384 | TSBTx | CACTCCATCGGCCA | 1863 | CACUCCAUCGGCC | 2019 | CBE_NGG_20 |
| | 4025 | TTCACT | | AUUCACU | | nt_4-9_003 |
| gRNA1385 | TSBTx | GTGGATTGCTTTGA | 1864 | GUGGAUUGCUUUG | 2020 | CBE_NGG_20 |
| | 4026 | GAAGGG | | AGAAGGG | | nt_4-9_003 |
| gRNA1386 | TSBTx | CCCCAGGGATCCTG | 1865 | CCCCAGGGAUCCU | 2021 | CBE_NGA_20 |
| | 4027 | CTCTCT | | GCUCUCU | | nt_4-9_006 |
| gRNA1387 | TSBTx | TCAAGACCAAAAGA | 1866 | UCAAGACCAAAAG | 2022 | CBE_NGA_20 |
| | 4028 | CTGTCA | | ACUGUCA | | nt_4-9_006 |
| gRNA1388 | TSBTx | GCACCTGCCAAGTG | 1867 | GCACCUGCCAAGU | 2023 | CBE_NGA_20 |
| | 4029 | AATGGC | | GAAUGGC | | nt_4-9_006 |
| gRNA1389 | TSBTx | CTGCCAAGTGAATG | 1868 | CUGCCAAGUGAAU | 2024 | CBE_NGA_20 |
| | 4030 | GCCGAT | | GGCCGAU | | nt_4-9_006 |
| gRNA1390 | TSBTx | GAATGGCCGATGGA | 1869 | GAAUGGCCGAUGG | 2025 | CBE_NGA_20 |
| | 4031 | GTGGGC | | AGUGGGC | | nt_4-9_006 |
| gRNA1391 | TSBTx | AGTGGGCAGACAGC | 1870 | AGUGGGCAGACAG | 2026 | CBE_NGA_20 |
| | 4032 | GATCTG | | CGAUCUG | | nt_4-9_006 |
| gRNA1392 | TSBTx | GGCAGCCAGTACCG | 1871 | GGCAGCCAGUACC | 2027 | CBE_NGA_20 |
| | 4033 | CCTTGA | | GCCUUGA | | nt_4-9_006 |
| gRNA1393 | TSBTx | TCCCAGCGGCGAAC | 1872 | UCCCAGCGGCGAA | 2028 | CBE_NGA_20 |
| | 4034 | GTGTCA | | CGUGUCA | | nt_4-9_006 |
| gRNA1394 | TSBTx | GTGTCAGGAAGGTG | 1873 | GUGUCAGGAAGGU | 2029 | CBE_NGA_20 |
| | 4035 | GCTCTT | | GGCUCUU | | nt_4-9_006 |
| gRNA1395 | TSBTx | AGGGGAACAACAGA | 1874 | AGGGGAACAACAG | 2030 | CBE_NGA_20 |
| | 4036 | AGCGGA | | AAGCGGA | | nt_4-9_006 |
| gRNA1396 | TSBTx | CCTCCAGGCAGTGT | 1875 | CCUCCAGGCAGUG | 2031 | CBE_NGA_20 |
| | 4037 | ACAGCA | | UACAGCA | | nt_4-9_006 |
| gRNA1397 | TSBTx | TTCTACCAAATGAT | 1876 | UUCUACCAAAUGA | 2032 | CBE_NGA_20 |
| | 4038 | CGGTAG | | UCGGUAG | | nt_4-9_006 |
| gRNA1398 | TSBTx | CTACCAAATGATCG | 1877 | CUACCAAAUGAUC | 2033 | CBE_NGA_20 |
| | 4039 | GTAGGG | | GGUAGGG | | nt_4-9_006 |
| gRNA1399 | TSBTx | CAAGCAACCATGGC | 1878 | CAAGCAACCAUGG | 2034 | CBE_NGA_20 |
| | 4040 | AGGCCA | | CAGGCCA | | nt_4-9_006 |
| gRNA1400 | TSBTx | TATGGCCAGACTAT | 1879 | UAUGGCCAGACUA | 2035 | CBE_NGA_20 |
| | 4041 | CAGGTG | | UCAGGUG | | nt_4-9_006 |
| gRNA1401 | TSBTx | CACTTGCCAGCAAC | 1880 | CACUUGCCAGCAA | 2036 | CBE_NGA_20 |
| | 4042 | AAAGTA | | CAAAGUA | | nt_4-9_006 |
| gRNA1402 | TSBTx | GCTCAATATGCCCC | 1881 | GCUCAAUAUGCCC | 2037 | CBE_NGA_20 |
| | 4043 | AGGCTA | | CAGGCUA | | nt_4-9_006 |
| gRNA1403 | TSBTx | CTTTCAAGTGCTGC | 1882 | CUUUCAAGUGCUG | 2038 | CBE_NGA_20 |
| | 4044 | CCTGGC | | CCCUGGC | | nt_4-9_006 |
| gRNA1404 | TSBTx | CCTTCAGCCAGGGC | 1883 | CCUUCAGCCAGGG | 2039 | CBE_NGA_20 |
| | 4045 | AGCACT | | CAGCACU | | nt_4-9_006 |
| gRNA1405 | TSBTx | CAGCCAGGGCAGCA | 1884 | CAGCCAGGGCAGC | 2040 | CBE_NGA_20 |
| | 4046 | CTTGAA | | ACUUGAA | | nt_4-9_006 |
| gRNA1406 | TSBTx | GCCTTTCTGTAGGG | 1885 | GCCUUUCUGUAGG | 2041 | CBE_NGA_20 |
| | 4047 | GAGGTG | | GGAGGUG | | nt_4-9_006 |
| gRNA1407 | TSBTx | AATGGGCCTATAAA | 1886 | AAUGGGCCUAUAA | 2042 | CBE_NGA_20 |
| | 4048 | GGACAA | | AGGACAA | | nt_4-9_006 |
| gRNA1408 | TSBTx | TGGGCCTATAAAGG | 1887 | UGGGCCUAUAAAG | 2043 | CBE_NGA_20 |
| | 4049 | ACAAGG | | GACAAGG | | nt_4-9_006 |
| gRNA1409 | TSBTx | GTGTTCCCAAACCA | 1888 | GUGUUCCCAAACC | 2044 | CBE_NGA_20 |
| | 4050 | TGCCAC | | AUGCCAC | | nt_4-9_006 |
| gRNA1410 | TSBTx | GTTCCCAAACCATG | 1889 | GUUCCCAAACCAU | 2045 | CBE_NGA_20 |
| | 4051 | CCACAG | | GCCACAG | | nt_4-9_006 |
| gRNA1411 | TSBTx | GCGGTTCCAGCCTT | 1890 | GCGGUUCCAGCCU | 2046 | CBE_NGA_20 |
| | 4052 | CAGGAG | | UCAGGAG | | nt_4-9_006 |
| gRNA1412 | TSBTx | TTGTGGTCTGTGGA | 1891 | UUGUGGUCUGUGG | 2047 | CBE_NGA_20 |
| | 4053 | GAGGGA | | AGAGGGA | | nt_4-9_006 |
| gRNA1413 | TSBTx | GAGTCTGGGAGAGT | 1892 | GAGUCUGGGAGAG | 2048 | CBE_NGA_20 |
| | 4054 | CAGAAA | | UCAGAAA | | nt_4-9_006 |
| gRNA1414 | TSBTx | CGCTCCAAGAGCCA | 1893 | CGCUCCAAGAGCC | 2049 | CBE_NGA_20 |
| | 4055 | CCTTCC | | ACCUUCC | | nt_4-9_006 |
| gRNA1415 | TSBTx | TACCCCGCTGCAGA | 1894 | UACCCCGCUGCAG | 2050 | CBE_NGA_20 |
| | 4056 | GGTATG | | AGGUAUG | | nt_4-9_006 |
| gRNA1416 | TSBTx | GGGCCAGTATTCCC | 1895 | GGGCCAGUAUUCC | 2051 | CBE_NGA_20 |
| | 4057 | CGTTCT | | CCGUUCU | | nt_4-9_006 |
| gRNA1417 | TSBTx | TGGATTGCTTTGAG | 1896 | UGGAUUGCUUUGA | 2052 | CBE_NGA_20 |
| | 4058 | AAGGGG | | GAAGGGG | | nt_4-9_006 |
| gRNA1418 | TSBTx | AGGGTGCTCCAGGA | 1897 | AGGGUGCUCCAGG | 2053 | CBE_NGA_20 |
| | 4059 | CCCCGT | | ACCCCGU | | nt_4-9_006 |
| gRNA1419 | TSBTx | CACCTGAAGAGAAA | 1898 | CACCUGAAGAGAA | 2054 | CBE_NGA_20 |
| | 4060 | GGCTGA | | AGGCUGA | | nt_4-9_006 |

TABLE 2E
Representative spacer and target site sequences relating to disrupting of a
complement_factor B splice site or introducing a new stop codon into a complement
factor B polynucleotide using base editing.

| | | Cas9 | alteration |
|---|---|---|---|
| Guide polynucleotide name | Editor | variant | description |
| GuideDesign10_CBE_NGA_20nt_4- | CBE_NGA_20nt_4- | spCas9 | Splice Site |
| 9_006_−_31947177_spCas9 | 9_006 | VRQR | |
| | | CBE | |
| GuideDesign10_CBE_NGA_20nt_4- | CBE_NGA_20nt_4- | spCas9 | Splice Site |
| 9_006_−_31948943_spCas9 | 9_006 | VRQR | |
| | | CBE | |
| GuideDesign10_CBE_NGA_20nt_4- | CBE_NGA_20nt_4- | spCas9 | Splice Site |
| 9_006_−_31949542_spCas9 | 9_006 | VRQR | |
| | | CBE | |
| GuideDesign10_CBE_NGA_20nt_4- | CBE_NGA_20nt_4- | spCas9 | Splice Site |
| 9_006_−_31950384_spCas9 | 9_006 | VRQR | |
| | | CBE | |
| GuideDesign10_CBE_NGA_20nt_4- | CBE_NGA_20nt_4- | spCas9 | Splice Site |
| 9_006_−_31950388_spCas9 | 9_006 | VRQR | |
| | | CBE | |
| GuideDesign10_CBE_NGA_20nt_4- | CBE_NGA_20nt_4- | spCas9 | Splice Site |
| 9_006_−_31951227_spCas9 | 9_006 | VRQR | |
| | | CBE | |
| GuideDesign10_CBE_NGC_20nt_4- | CBE_NGC_20nt_4- | spCas9 | Splice Site |
| 9_009_−_31946270_spCas9 | 9_009 | NGC | |
| | | CBE | |
| GuideDesign10_CBE_NGC_20nt_4- | CBE_NGC_20nt_4- | spCas9 | Splice Site |
| 9_009_−_31947173_spCas9 | 9_009 | NGC | |
| | | CBE | |
| GuideDesign10_CBE_NGC_20nt_4- | CBE_NGC_20nt_4- | spCas9 | Splice Site |
| 9_009_−_31950129_spCas9 | 9_009 | NGC | |
| | | CBE | |
| GuideDesign10_CBE_NGC_20nt_4- | CBE_NGC_20nt_4- | spCas9 | Splice Site |
| 9_009_−_31951854_spCas9 | 9_009 | NGC | |
| | | CBE | |
| GuideDesign10_CBE_NGG_20nt_4- | CBE_NGG_20nt_4- | spCas9 | Splice Site |
| 9_003_−_31946271_spCas9 | 9_003 | CBE | |
| GuideDesign10_CBE_NGG_20nt_4- | CBE_NGG_20nt_4- | spCas9 | Splice Site |
| 9_003_−_31948947_spCas9 | 9_003 | CBE | |
| GuideDesign10_CBE_NGG_20nt_4- | CBE_NGG_20nt_4- | spCas9 | Splice Site |
| 9_003_−_31949325_spCas9 | 9_003 | CBE | |
| GuideDesign10_CBE_NGG_20nt_4- | CBE_NGG_20nt_4- | spCas9 | Splice Site |
| 9_003_−_31950130_spCas9 | 9_003 | CBE | |
| GuideDesign10_CBE_NGG_20nt_4- | CBE_NGG_20nt_4- | spCas9 | Splice Site |
| 9_003_−_31950926_spCas9 | 9_003 | CBE | |
| GuideDesign10_CBE_NGG_20nt_4- | CBE_NGG_20nt_4- | spCas9 | Splice Site |
| 9_003_−_31951456_spCas9 | 9_003 | CBE | |
| GuideDesign3_ABE_NGA_20nt_3- | ABE_NGA_20nt_3- | [′spCas9 | Splice Site |
| 12_019_−_31946273_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_−_31947177_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_−_31947511_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_−_31948064_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_−_31948500_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_−_31948943_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_−_31948946_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_−_31948949_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_−_31949542_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_−_31950136_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_−_31950384_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_−_31950388_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_−_31951227_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_−_31951591_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_+30_31947000_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_+_31947734_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_+_31947933_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_+_31947939_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_+_31948369_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_+30_31948818_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_+_31950044_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_+_31950277_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_+_31950611_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_+_31951331_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_+_31951333_spCas9 | 12_019 | VRQR | |
| | | IBE′] | |
| GuideDesign3_ABE_NGA_20nt_3- | ABE_NGA_20nt_3- | [′spCas9 | Splice Site |
| 12_019_+_31951335_spCas9 | 12_019 | VRQR | |
| | | IBE′] | |
| GuideDesign3_ABE_NGA_20nt_3- | ABE_NGA_20nt_3- | [′spCas9 | Splice Site |
| 9_005_−_31948064_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_−_31948946_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_+_31950611_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_+_31951331_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_+_31951333_spCas9 | 9_005 | VRQR | |
| | | ABE′] | |
| GuideDesign3_ABE_NGC_20nt_3- | ABE_NGC_20nt_3- | [′spCas9 | Splice Site |
| 12_020_−_31946270_spCas9 | 12_020 | NGC | |
| | | IBE′] | |
| GuideDesign3_ABE_NGC_20nt_3- | ABE_NGC_20nt_3- | [′spCas9 | Splice Site |
| 12_020_−_31946593_spCas9 | 12_020 | NGC | |
| | | IBE′] | |
| GuideDesign3_ABE_NGC_20nt_3- | ABE_NGC_20nt_3- | [′spCas9 | Splice Site |
| 12_020_−_31947173_spCas9 | 12_020 | NGC | |
| | | IBE′] | |
| GuideDesign3_ABE_NGC_20nt_3- | ABE_NGC_20nt_3- | [′spCas9 | Splice Site |
| 12_020_−_31947507_spCas9 | 12_020 | NGC | |
| | | IBE′] | |
| GuideDesign3_ABE_NGC_20nt_3- | ABE_NGC_20nt_3- | [′spCas9 | Splice Site |
| 12_020_−_31947832_spCas9 | 12_020 | NGC | |
| | | IBE′] | |
| GuideDesign3_ABE_NGC_20nt_3- | ABE_NGC_20nt_3- | [′spCas9 | Splice Site |
| 12_020_−_31950129_spCas9 | 12_020 | NGC | |
| | | IBE′] | |
| GuideDesign3_ABE_NGC_20nt_3- | ABE_NGC_20nt_3- | [′spCas9 | Splice Site |
| 12_020_−_31950759_spCas9 | 12_020 | NGC | |
| | | IBE′] | |
| GuideDesign3_ABE_NGC_20nt_3- | ABE_NGC_20nt_3- | [′spCas9 | Splice Site |
| 12_020_−_31950932_spCas9 | 12_020 | NGC | |
| | | IBE′] | |
| GuideDesign3_ABE_NGC_20nt_3- | ABE_NGC_20nt_3- | [′spCas9 | Splice Site |
| 12_020_+_31947335_spCas9 | 12_020 | NGC | |
| | | IBE′] | |
| GuideDesign3_ABE_NGC_20nt_3- | ABE_NGC_20nt_3- | [′spCas9 | Splice Site |
| 12_020_+_31947936_spCas9 | 12_020 | NGC | |
| | | IBE′] | |
| GuideDesign3_ABE_NGC_20nt_3- | ABE_NGC_20nt_3- | [′spCas9 | Splice Site |
| 12_020_+_31949236_spCas9 | 12_020 | NGC | |
| | | IBE′] | |
| GuideDesign3_ABE_NGC_20nt_3- | ABE_NGC_20nt_3- | [′spCas9 | Splice Site |
| 12_020_+_31950607_spCas9 | 12_020 | NGC | |
| | | IBE′] | |
| GuideDesign3_ABE_NGC_20nt_3- | ABE_NGC_20nt_3- | [′spCas9 | Splice Site |
| 12_020_+_31950863_spCas9 | 12_020 | NGC | |
| | | IBE′] | |
| GuideDesign3_ABE_NGC_20nt_3- | ABE_NGC_20nt_3- | [′spCas9 | Splice Site |
| 12_020_+30_31951132_spCas9 | 12_020 | NGC | |
| | | IBE′] | |
| GuideDesign3_ABE_NGC_20nt_3- | ABE_NGC_20nt_3- | [′spCas9 | Splice Site |
| 12_020_+_31951139_spCas9 | 12_020 | NGC | |
| | | IBE′] | |
| GuideDesign3_ABE_NGC_20nt_3- | ABE_NGC_20nt_3- | [′spCas9 | Splice Site |
| 12_020_+_31951542_spCas9 | 12_020 | NGC | |
| | | IBE′] | |
| GuideDesign3_ABE_NGC_20nt_3- | ABE_NGC_20nt_3- | [′spCas9 | Splice Site |
| 12_020_+_31951545_spCas9 | 12_020 | NGC | |
| | | IBE′] | |
| GuideDesign3_ABE_NGC_20nt_3- | ABE_NGC_20nt_3- | [′spCas9 | Splice Site |
| 12_020_+_31951866_spCas9 | 12_020 | NGC | |
| | | IBE′] | |
| GuideDesign3_ABE_NGC_20nt_3- | ABE_NGC_20nt_3- | [′spCas9 | Splice Site |
| 9_008_−_31946593_spCas9 | 9_008 | NGC | |
| | | ABE′] | |
| GuideDesign3_ABE_NGC_20nt_3- | ABE_NGC_20nt_3- | [′spCas9 | Splice Site |
| 9_008_−_31947507_spCas9 | 9_008 | NGC | |
| | | ABE′] | |
| GuideDesign3_ABE_NGC_20nt_3- | ABE_NGC_20nt_3- | [′spCas9 | Splice Site |
| 9_008_+30_31947936_spCas9 | 9_008 | NGC | |
| | | ABE′] | |
| GuideDesign3_ABE_NGC_20nt_3- | ABE_NGC_20nt_3- | [′spCas9 | Splice Site |
| 9_008_+_31951139_spCas9 | 9_008 | NGC | |
| | | ABE′] | |
| GuideDesign3_ABE_NGC_20nt_3- | ABE_NGC_20nt_3- | [′spCas9 | Splice Site |
| 9_008_+30_31951545_spCas9 | 9_008 | NGC | |
| | | ABE′] | |
| GuideDesign3_ABE_NGG_20nt_3- | ABE_NGG_20nt_3- | [′spCas9 | Splice Site |
| 12_018_−_31946271_spCas9 | 12_018 | IBE′] | |
| GuideDesign3_ABE_NGG_20nt_3- | ABE_NGG_20nt_3- | [′spCas9 | Splice Site |
| 12_018_−_31947508_spCas9 | 12_018 | IBE′] | |
| GuideDesign3_ABE_NGG_20nt_3- | ABE_NGG_20nt_3- | [′spCas9 | Splice Site |
| 12_018_−_31948947_spCas9 | 12_018 | IBE′] | |
| GuideDesign3_ABE_NGG_20nt_3- | ABE_NGG_20nt_3- | [′spCas9 | Splice Site |
| 12_018_−_31949325_spCas9 | 12_018 | IBE′] | |
| GuideDesign3_ABE_NGG_20nt_3- | ABE_NGG_20nt_3- | [′spCas9 | Splice Site |
| 12_018_−_31949546_spCas9 | 12_018 | IBE′] | |
| GuideDesign3_ABE_NGG_20nt_3- | ABE_NGG_20nt_3- | [′spCas9 | Splice Site |
| 12_018_−_31950130_spCas9 | 12_018 | IBE′] | |
| GuideDesign3_ABE_NGG_20nt_3- | ABE_NGG_20nt_3- | [′spCas9 | Splice Site |
| 12_018_−_31950760_spCas9 | 12_018 | IBE′] | |
| GuideDesign3_ABE_NGG_20nt_3- | ABE_NGG_20nt_3- | [′spCas9 | Splice Site |
| 12_018_−_31950926_spCas9 | 12_018 | IBE′] | |
| GuideDesign3_ABE_NGG_20nt_3- | ABE_NGG_20nt_3- | [′spCas9 | Splice Site |
| 12_018_−_31950933_spCas9 | 12_018 | IBE′] | |
| GuideDesign3_ABE_NGG_20nt_3- | ABE_NGG_20nt_3- | [′spCas9 | Splice Site |
| 12_018_−_31951456_spCas9 | 12_018 | IBE′] | |
| GuideDesign3_ABE_NGG_20nt_3- | ABE_NGG_20nt_3- | [′spCas9 | Splice Site |
| 12_018_−_31951457_spCas9 | 12_018 | IBE′] | |
| GuideDesign3_ABE_NGG_20nt_3- | ABE_NGG_20nt_3- | [′spCas9 | Splice Site |
| 12_018_−_31951458_spCas9 | 12_018 | IBE′] | |
| GuideDesign3_ABE_NGG_20nt_3- | ABE_NGG_20nt_3- | [′spCas9 | Splice Site |
| 12_018_+_31947938_spCas9 | 12_018 | IBE′] | |
| GuideDesign3_ABE_NGG_20nt_3- | ABE_NGG_20nt_3- | [′spCas9 | Splice Site |
| 12_018_+_31948364_spCas9 | 12_018 | IBE′] | |
| GuideDesign3_ABE_NGG_20nt_3- | ABE_NGG_20nt_3- | [′spCas9 | Splice Site |
| 12_018_+_31948825_spCas9 | 12_018 | IBE′] | |
| GuideDesign3_ABE_NGG_20nt_3- | ABE_NGG_20nt_3- | [′spCas9 | Splice Site |
| 12_018_+_31949234_spCas9 | 12_018 | IBE′] | |
| GuideDesign3_ABE_NGG_20nt_3- | ABE_NGG_20nt_3- | [′spCas9 | Splice Site |
| 12_018_+_31949235_spCas9 | 12_018 | IBE′] | |
| GuideDesign3_ABE_NGG_20nt_3- | ABE_NGG_20nt_3- | [′spCas9 | Splice Site |
| 12_018_+_31949238_spCas9 | 12_018 | IBE′] | |
| GuideDesign3_ABE_NGG_20nt_3- | ABE_NGG_20nt_3- | [′spCas9 | Splice Site |
| 12_018_+_31949412_spCas9 | 12_018 | IBE′] | |
| GuideDesign3_ABE_NGG_20nt_3- | ABE_NGG_20nt_3- | [′spCas9 | Splice Site |
| 12_018_+_31949413_spCas9 | 12_018 | IBE′] | |
| GuideDesign3_ABE_NGG_20nt_3- | ABE_NGG_20nt_3- | [′spCas9 | Splice Site |
| 12_018_+_31949414_spCas9 | 12_018 | IBE′] | |
| GuideDesign3_ABE_NGG_20nt_3- | ABE_NGG_20nt_3- | [′spCas9 | Splice Site |
| 12_018_+_31950275_spCas9 | 12_018 | IBE′] | |
| GuideDesign3_ABE_NGG_20nt_3- | ABE_NGG_20nt_3- | [′spCas9 | Splice Site |
| 12_018_+_31950276_spCas9 | 12_018 | IBE′] | |
| GuideDesign3_ABE_NGG_20nt_3- | ABE_NGG_20nt_3- | [′spCas9 | Splice Site |
| 12_018_+_31950609_spCas9 | 12_018 | IBE′] | |
| GuideDesign3_ABE_NGG_20nt_3- | ABE_NGG_20nt_3- | [′spCas9 | Splice Site |
| 12_018_+_31950610_spCas9 | 12_018 | IBE′] | |
| GuideDesign3_ABE_NGG_20nt_3- | ABE_NGG_20nt_3- | [′spCas9 | Splice Site |
| 12_018_+_31951541_spCas9 | 12_018 | IBE′] | |
| GuideDesign3_ABE_NGG_20nt_3- | ABE_NGG_20nt_3- | [′spCas9 | Splice Site |
| 12_018_+_31951544_spCas9 | 12_018 | IBE′] | |
| GuideDesign3_ABE_NGG_20nt_3- | ABE_NGG_20nt_3- | [′spCas9 | Splice Site |
| 12_018_+_31951869_spCas9 | 12_018 | IBE′] | |
| GuideDesign3_ABE_NGG_20nt_3- | ABE_NGG_20nt_3- | [′spCas9 | Splice Site |
| 12_018_+_31951870_spCas9 | 12_018 | IBE′] | |
| GuideDesign3_ABE_NGG_20nt_3- | ABE_NGG_20nt_3- | [′spCas9 | Splice Site |
| 9_002_−_31951457_spCas9 | 9_002 | ABE′] | |
| GuideDesign3_ABE_NGG_20nt_3- | ABE_NGG_20nt_3- | [′spCas9 | Splice Site |
| 9_002_−_31951458_spCas9 | 9_002 | ABE′] | |
| GuideDesign3_ABE_NGG_20nt_3- | ABE_NGG_20nt_3- | [′spCas9 | Splice Site |
| 9_002_+_31947938_spCas9 | 9_002 | ABE′] | |
| GuideDesign3_ABE_NGG_20nt_3- | ABE_NGG_20nt_3- | [′spCas9 | Splice Site |
| 9_002_+_31949234_spCas9 | 9_002 | ABE′] | |
| GuideDesign3_ABE_NNNRRT_21nt_5- | ABE_NNNRRT_21nt_5- | [′saCas9 | Splice Site |
| 14_014_−_31948945_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31949323_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31950382_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31951226_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31951455_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_+_31948361_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_+_31949411_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_+_31950034_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_+_31950042_saCas9 | 14_014 | KKH | |
| | | ABE′] | |
| GuideDesign3_ABE_VTTN_22nt_5- | ABE_VTTN_22nt_5- | [′cas12b | Splice Site |
| 9_017_−_31946593_cas12b | 9_017 | ABE′] | |
| GuideDesign3_ABE_VTTN_22nt_5- | ABE_VTTN_22nt_5- | [′cas12b | Splice Site |
| 9_017_−_31950134_cas12b | 9_017 | ABE′] | |
| GuideDesign3_ABE_VTTN_22nt_5- | ABE_VTTN_22nt_5- | [′cas12b | Splice Site |
| 9_017_−_31950390_cas12b | 9_017 | ABE′] | |
| GuideDesign3_ABE_VTTN_22nt_5- | ABE_VTTN_22nt_5- | [′cas12b | Splice Site |
| 9_017_−_31951457_cas12b | 9_017 | ABE′] | |
| GuideDesign3_ABE_VTTN_22nt_5- | ABE_VTTN_22nt_5- | [′cas12b | Splice Site |
| 9_017_+_31946360_cas12b | 9_017 | ABE′] | |
| GuideDesign3_ABE_VTTN_22nt_5- | ABE_VTTN_22nt_5- | [′cas12b | Splice Site |
| 9_017_+_31946996_cas12b | 9_017 | ABE′] | |
| GuideDesign3_ABE_VTTN_22nt_5- | ABE_VTTN_22nt_5- | [′cas12b | Splice Site |
| 9_017_+_31950037_cas12b | 9_017 | ABE′] | |
| GuideDesign3_ABE_VTTN_22nt_5- | ABE_VTTN_22nt_5- | [′cas12b | Splice Site |
| 9_017_+_31950272_cas12b | 9_017 | ABE′] | |
| GuideDesign3_ABE_VTTN_22nt_5- | ABE_VTTN_22nt_5- | [′cas12b | Splice Site |
| 9_017_+_31950853_cas12b | 9_017 | ABE′] | |
| GuideDesign3_ABE_VTTN_22nt_5- | ABE_VTTN_22nt_5- | [′cas12b | Splice Site |
| 9_017_+_31951133_cas12b | 9_017 | ABE′] | |
| GuideDesign3_CBE_NNGRRT_21nt_3- | CBE_NNGRRT_21nt_3- | [′saCas9 | Splice Site |
| 12_012_−_31948945_saCas9 | 12_012 | CBE′] | |
| GuideDesign3_CBE_NNGRRT_21nt_3- | CBE_NNGRRT_21nt_3- | [′saCas9 | Splice Site |
| 12_012_−_31949323_saCas9 | 12_012 | CBE′] | |
| GuideDesign3_CBE_NNGRRT_21nt_3- | CBE_NNGRRT_21nt_3- | [′saCas9 | Splice Site |
| 12_012_−_31950382_saCas9 | 12_012 | CBE′] | |
| GuideDesign3_CBE_NNGRRT_21nt_3- | CBE_NNGRRT_21nt_3- | [′saCas9 | Splice Site |
| 12_012_−_31951455_saCas9 | 12_012 | CBE′] | |
| GuideDesign3_CBE_NNNRRT_21nt_3- | CBE_NNNRRT_21nt_3- | [′saCas9 | Splice Site |
| 12_015_−_31947176_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31947927_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31948942_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31948945_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31949323_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31949397_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31950031_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31950382_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31950387_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31950925_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31950928_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31951226_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31951455_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31951856_saCas9 | 12_015 | KKH | |
| | | CBE′] | |
| GuideDesign10_CBE_NGA_20nt_4- | CBE_NGA_20nt_4- | spCas9 | Splice Site |
| 9_006_−_31946352_spCas9 | 9_006 | VRQR | |
| | | CBE | |
| GuideDesign10_CBE_NGA_20nt_4- | CBE_NGA_20nt_4- | spCas9 | Splice Site |
| 9_006_−_31946990_spCas9 | 9_006 | VRQR | |
| | | CBE | |
| GuideDesign10_CBE_NGA_20nt_4- | CBE_NGA_20nt_4- | spCas9 | Splice Site |
| 9_006_−_31947331_spCas9 | 9_006 | VRQR | |
| | | CBE | |
| GuideDesign10_CBE_NGA_20nt_4- | CBE_NGA_20nt_4- | spCas9 | Splice Site |
| 9_006_−_31947722_spCas9 | 9_006 | VRQR | |
| | | CBE | |
| GuideDesign10_CBE_NGA_20nt_4- | CBE_NGA_20nt_4- | spCas9 | Splice Site |
| 9_006_−_31950849_spCas9 | 9_006 | VRQR | |
| | | CBE | |
| GuideDesign10_CBE_NGA_20nt_4- | CBE_NGA_20nt_4- | spCas9 | Splice Site |
| 9_006_−_31950851_spCas9 | 9_006 | VRQR | |
| | | CBE | |
| GuideDesign10_CBE_NGA_20nt_4- | CBE_NGA_20nt_4- | spCas9 | Splice Site |
| 9_006_−_31951323_spCas9 | 9_006 | VRQR | |
| | | CBE | |
| GuideDesign10_CBE_NGG_20nt_4- | CBE_NGG_20nt_4- | spCas9 | Splice Site |
| 9_003_−_31946991_spCas9 | 9_003 | CBE | |
| GuideDesign10_CBE_NGG_20nt_4- | CBE_NGG_20nt_4- | spCas9 | Splice Site |
| 9_003_−_31949226_spCas9 | 9_003 | CBE | |
| GuideDesign10_CBE_NGG_20nt_4- | CBE_NGG_20nt_4- | spCas9 | Splice Site |
| 9_003_−_31949399_spCas9 | 9_003 | CBE | |
| GuideDesign10_CBE_NGG_20nt_4- | CBE_NGG_20nt_4- | spCas9 | Splice Site |
| 9_003_−_31949403_spCas9 | 9_003 | CBE | |
| GuideDesign10_CBE_NGG_20nt_4- | CBE_NGG_20nt_4- | spCas9 | Splice Site |
| 9_003_−_31950600_spCas9 | 9_003 | CBE | |
| GuideDesign10_CBE_NGG_20nt_4- | CBE_NGG_20nt_4- | spCas9 | Splice Site |
| 9_003_−_31950852_spCas9 | 9_003 | CBE | |
| GuideDesign10_CBE_NGG_20nt_4- | CBE_NGG_20nt_4- | spCas9 | Splice Site |
| 9_003_−_31951324_spCas9 | 9_003 | CBE | |
| GuideDesign10_CBE_NGG_20nt_4- | CBE_NGG_20nt_4- | spCas9 | Splice Site |
| 9_003_−_31951857_spCas9 | 9_003 | CBE | |
| GuideDesign3_ABE_NGA_20nt_3- | ABE_NGA_20nt_3- | [′spCas9 | Splice Site |
| 9_005_−_31947177_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_−_31947177_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_−_31948943_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_−_31948943_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_−_31949542_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_−_31949542_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_−_31950384_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_−_31950384_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_−_31950388_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_−_31950388_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_−_31951227_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_−_31951227_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_−_31951591_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_−_31951591_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_+_31947000_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_+_31947000_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_+_31947734_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_+_31947734_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_+_31947939_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_+_31947939_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_+_31948369_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_+_31948369_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_+_31950044_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_+_31950044_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_+_31950277_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_+_31950277_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_+_31951335_spCas9 | 9_005 | VRQR | |
| | | ABE′] | |
| GuideDesign3_ABE_NGA_20nt_3- | ABE_NGA_20nt_3- | [′spCas9 | Splice Site |
| 9_005_+_31951335_spCas9 | 9_005 | VRQR | |
| | | ABE′] | |
| GuideDesign3_ABE_NGC_20nt_3- | ABE_NGC_20nt_3- | [′spCas9 | Splice Site |
| 9_008_−_31946270_spCas9 | 9_008 | NGC | |
| | | ABE′] | |
| GuideDesign3_ABE_NGC_20nt_3- | ABE_NGC_20nt_3- | [′spCas9 | Splice Site |
| 9_008_−_31946270_spCas9 | 9_008 | NGC | |
| | | ABE′] | |
| GuideDesign3_ABE_NGC_20nt_3- | ABE_NGC_20nt_3- | [′spCas9 | Splice Site |
| 9_008_−_31947173_spCas9 | 9_008 | NGC | |
| | | ABE′] | |
| GuideDesign3_ABE_NGC_20nt_3- | ABE_NGC_20nt_3- | [′spCas9 | Splice Site |
| 9_008_−_31947173_spCas9 | 9_008 | NGC | |
| | | ABE′] | |
| GuideDesign3_ABE_NGC_20nt_3- | ABE_NGC_20nt_3- | [′spCas9 | Splice Site |
| 9_008_−_31950129_spCas9 | 9_008 | NGC | |
| | | ABE′] | |
| GuideDesign3_ABE_NGC_20nt_3- | ABE_NGC_20nt_3- | [′spCas9 | Splice Site |
| 9_008_−_31950129_spCas9 | 9_008 | NGC | |
| | | ABE′] | |
| GuideDesign3_ABE_NGC_20nt_3- | ABE_NGC_20nt_3- | [′spCas9 | Splice Site |
| 9_008_−_31950759_spCas9 | 9_008 | NGC | |
| | | ABE′] | |
| GuideDesign3_ABE_NGC_20nt_3- | ABE_NGC_20nt_3- | [′spCas9 | Splice Site |
| 9_008_−_31950759_spCas9 | 9_008 | NGC | |
| | | ABE′] | |
| GuideDesign3_ABE_NGC_20nt_3- | ABE_NGC_20nt_3- | [′spCas9 | Splice Site |
| 9_008_+_31949236_spCas9 | 9_008 | NGC | |
| | | ABE′] | |
| GuideDesign3_ABE_NGC_20nt_3- | ABE_NGC_20nt_3- | [′spCas9 | Splice Site |
| 9_008_+_31949236_spCas9 | 9_008 | NGC | |
| | | ABE′] | |
| GuideDesign3_ABE_NGC_20nt_3- | ABE_NGC_20nt_3- | [′spCas9 | Splice Site |
| 9_008_+_31950863_spCas9 | 9_008 | NGC | |
| | | ABE′] | |
| GuideDesign3_ABE_NGC_20nt_3- | ABE_NGC_20nt_3- | [′spCas9 | Splice Site |
| 9_008_+_31950863_spCas9 | 9_008 | NGC | |
| | | ABE′] | |
| GuideDesign3_ABE_NGC_20nt_3- | ABE_NGC_20nt_3- | [′spCas9 | Splice Site |
| 9_008_+_31951866_spCas9 | 9_008 | NGC | |
| | | ABE′] | |
| GuideDesign3_ABE_NGC_20nt_3- | ABE_NGC_20nt_3- | [′spCas9 | Splice Site |
| 9_008_+_31951866_spCas9 | 9_008 | NGC | |
| | | ABE′] | |
| GuideDesign3_ABE_NGG_20nt_3- | ABE_NGG_20nt_3- | [′spCas9 | Splice Site |
| 9_002_−_31946271_spCas9 | 9_002 | ABE′] | |
| GuideDesign3_ABE_NGG_20nt_3- | ABE_NGG_20nt_3- | [′spCas9 | Splice Site |
| 9_002_−_31946271_spCas9 | 9_002 | ABE′] | |
| GuideDesign3_ABE_NGG_20nt_3- | ABE_NGG_20nt_3- | [′spCas9 | Splice Site |
| 9_002_−_31947508_spCas9 | 9_002 | ABE′] | |
| GuideDesign3_ABE_NGG_20nt_3- | ABE_NGG_20nt_3- | [′spCas9 | Splice Site |
| 9_002_−_31947508_spCas9 | 9_002 | ABE′] | |
| GuideDesign3_ABE_NGG_20nt_3- | ABE_NGG_20nt_3- | [′spCas9 | Splice Site |
| 9_002_−_31948947_spCas9 | 9_002 | ABE′] | |
| GuideDesign3_ABE_NGG_20nt_3- | ABE_NGG_20nt_3- | [′spCas9 | Splice Site |
| 9_002_−_31948947_spCas9 | 9_002 | ABE′] | |
| GuideDesign3_ABE_NGG_20nt_3- | ABE_NGG_20nt_3- | [′spCas9 | Splice Site |
| 9_002_−_31949325_spCas9 | 9_002 | ABE′] | |
| GuideDesign3_ABE_NGG_20nt_3- | ABE_NGG_20nt_3- | [′spCas9 | Splice Site |
| 9_002_−_31949325_spCas9 | 9_002 | ABE′] | |
| GuideDesign3_ABE_NGG_20nt_3- | ABE_NGG_20nt_3- | [′spCas9 | Splice Site |
| 9_002_−_31950130_spCas9 | 9_002 | ABE′] | |
| GuideDesign3_ABE_NGG_20nt_3- | ABE_NGG_20nt_3- | [′spCas9 | Splice Site |
| 9_002_−_31950130_spCas9 | 9_002 | ABE′] | |
| GuideDesign3_ABE_NGG_20nt_3- | ABE_NGG_20nt_3- | [′spCas9 | Splice Site |
| 9_002_−_31950926_spCas9 | 9_002 | ABE′] | |
| GuideDesign3_ABE_NGG_20nt_3- | ABE_NGG_20nt_3- | [′spCas9 | Splice Site |
| 9_002_−_31950926_spCas9 | 9_002 | ABE′] | |
| GuideDesign3_ABE_NGG_20nt_3- | ABE_NGG_20nt_3- | [′spCas9 | Splice Site |
| 9_002_−_31951456_spCas9 | 9_002 | ABE′] | |
| GuideDesign3_ABE_NGG_20nt_3- | ABE_NGG_20nt_3- | [′spCas9 | Splice Site |
| 9_002_−_31951456_spCas9 | 9_002 | ABE′] | |
| GuideDesign3_ABE_NGG_20nt_3- | ABE_NGG_20nt_3- | [′spCas9 | Splice Site |
| 9_002_+_31948364_spCas9 | 9_002 | ABE′] | |
| GuideDesign3_ABE_NGG_20nt_3- | ABE_NGG_20nt_3- | [′spCas9 | Splice Site |
| 9_002_+_31948364_spCas9 | 9_002 | ABE′] | |
| GuideDesign3_ABE_NGG_20nt_3- | ABE_NGG_20nt_3- | [′spCas9 | Splice Site |
| 9_002_+_31948825_spCas9 | 9_002 | ABE′] | |
| GuideDesign3_ABE_NGG_20nt_3- | ABE_NGG_20nt_3- | [′spCas9 | Splice Site |
| 9_002_+_31948825_spCas9 | 9_002 | ABE′] | |
| GuideDesign3_ABE_NGG_20nt_3- | ABE_NGG_20nt_3- | [′spCas9 | Splice Site |
| 9_002_+_31949235_spCas9 | 9_002 | ABE′] | |
| GuideDesign3_ABE_NGG_20nt_3- | ABE_NGG_20nt_3- | [′spCas9 | Splice Site |
| 9_002_+_31949235_spCas9 | 9_002 | ABE′] | |
| GuideDesign3_ABE_NGG_20nt_3- | ABE_NGG_20nt_3- | [′spCas9 | Splice Site |
| 9_002_+_31949238_spCas9 | 9_002 | ABE′]T | |
| GuideDesign3_ABE_NGG_20nt_3- | ABE_NGG_20nt_3- | [′spCas9 | Splice Site |
| 9_002_+_31949238_spCas9 | 9_002 | ABE′] | |
| GuideDesign3_ABE_NGG_20nt_3- | ABE_NGG_20nt_3- | [′spCas9 | Splice Site |
| 9_002_+_31949412_spCas9 | 9_002 | ABE′] | |
| GuideDesign3_ABE_NGG_20nt_3- | ABE_NGG_20nt_3- | [′spCas9 | Splice Site |
| 9_002_+_31949412_spCas9 | 9_002 | ABE′] | |
| GuideDesign3_ABE_NGG_20nt_3- | ABE_NGG_20nt_3- | [′spCas9 | Splice Site |
| 9_002_+_31949413_spCas9 | 9_002 | ABE′] | |
| GuideDesign3_ABE_NGG_20nt_3- | ABE_NGG_20nt_3- | [′spCas9 | Splice Site |
| 9_002_+_31949413_spCas9 | 9_002 | ABE′] | |
| GuideDesign3_ABE_NGG_20nt_3- | ABE_NGG_20nt_3- | [′spCas9 | Splice Site |
| 9_002_+_31949414_spCas9 | 9_002 | ABE′] | |
| GuideDesign3_ABE_NGG_20nt_3- | ABE_NGG_20nt_3- | [′spCas9 | Splice Site |
| 9_002_+_31949414_spCas9 | 9_002 | ABE′] | |
| GuideDesign3_ABE_NGG_20nt_3- | ABE_NGG_20nt_3- | [′spCas9 | Splice Site |
| 9_002_+_31950275_spCas9 | 9_002 | ABE′] | |
| GuideDesign3_ABE_NGG_20nt_3- | ABE_NGG_20nt_3- | [′spCas9 | Splice Site |
| 9_002_+_31950275_spCas9 | 9_002 | ABE′] | |
| GuideDesign3_ABE_NGG_20nt_3- | ABE_NGG_20nt_3- | [′spCas9 | Splice Site |
| 9_002_+_31950276_spCas9 | 9_002 | ABE′] | |
| GuideDesign3_ABE_NGG_20nt_3- | ABE_NGG_20nt_3- | [′spCas9 | Splice Site |
| 9_002_+_31950276_spCas9 | 9_002 | ABE′] | |
| GuideDesign3_ABE_NGG_20nt_3- | ABE_NGG_20nt_3- | [′spCas9 | Splice Site |
| 9_002_+_31950609_spCas9 | 9_002 | ABE′] | |
| GuideDesign3_ABE_NGG_20nt_3- | ABE_NGG_20nt_3- | [′spCas9 | Splice Site |
| 9_002_+_31950609_spCas9 | 9_002 | ABE′] | |
| GuideDesign3_ABE_NGG_20nt_3- | ABE_NGG_20nt_3- | [′spCas9 | Splice Site |
| 9_002_+_31950610_spCas9 | 9_002 | ABE′] | |
| GuideDesign3_ABE_NGG_20nt_3- | ABE_NGG_20nt_3- | [′spCas9 | Splice Site |
| 9_002_+_31950610_spCas9 | 9_002 | ABE′] | |
| GuideDesign3_ABE_NGG_20nt_3- | ABE_NGG_20nt_3- | [′spCas9 | Splice Site |
| 9_002_+_31951544_spCas9 | 9_002 | ABE′] | |
| GuideDesign3_ABE_NGG_20nt_3- | ABE_NGG_20nt_3- | [′spCas9 | Splice Site |
| 9_002_+_31951544_spCas9 | 9_002 | ABE′] | |
| GuideDesign3_ABE_NGG_20nt_3- | ABE_NGG_20nt_3- | [′spCas9 | Splice Site |
| 9_002_+_31951869_spCas9 | 9_002 | ABE′] | |
| GuideDesign3_ABE_NGG_20nt_3- | ABE_NGG_20nt_3- | [′spCas9 | Splice Site |
| 9_002_+_31951869_spCas9 | 9_002 | ABE′] | |
| GuideDesign3_ABE_NGG_20nt_3- | ABE_NGG_20nt_3- | [′spCas9 | Splice Site |
| 9_002_+_31951870_spCas9 | 9_002 | ABE′] | |
| GuideDesign3_ABE_NGG_20nt_3- | ABE_NGG_20nt_3- | [′spCas9 | Splice Site |
| 9_002_+_31951870_spCas9 | 9_002 | ABE′] | |
| GuideDesign3_ABE_NNGRRT_21nt_5- | ABE_NNGRRT_21nt_5- | [′saCas9 | Splice Site |
| 14_011_−_31948945_saCas9 | 14_011 | ABE′] | |
| GuideDesign3_ABE_NNGRRT_21nt_5- | ABE_NNGRRT_21nt_5- | [′saCas9 | Splice Site |
| 14_011_−_31948945_saCas9 | 14_011 | ABE′] | |
| GuideDesign3_ABE_NNGRRT_21nt_5- | ABE_NNGRRT_21nt_5- | [′saCas9 | Splice Site |
| 14_011_−_31949323_saCas9 | 14_011 | ABE′] | |
| GuideDesign3_ABE_NNGRRT_21nt_5- | ABE_NNGRRT_21nt_5- | [′saCas9 | Splice Site |
| 14_011_−_31949323_saCas9 | 14_011 | ABE′] | |
| GuideDesign3_ABE_NNGRRT_21nt_5- | ABE_NNGRRT_21nt_5- | [′saCas9 | Splice Site |
| 14_011_−_31950382_saCas9 | 14_011 | ABE′] | |
| GuideDesign3_ABE_NNGRRT_21nt_5- | ABE_NNGRRT_21nt_5- | [′saCas9 | Splice Site |
| 14_011_−_31950382_saCas9 | 14_011 | ABE′] | |
| GuideDesign3_ABE_NNGRRT_21nt_5- | ABE_NNGRRT_21nt_5- | [′saCas9 | Splice Site |
| 14_011_−_31951455_saCas9 | 14_011 | ABE′] | |
| GuideDesign3_ABE_NNGRRT_21nt_5- | ABE_NNGRRT_21nt_5- | [′saCas9 | Splice Site |
| 14_011_−_31951455_saCas9 | 14_011 | ABE′] | |
| GuideDesign3_ABE_NNGRRT_21nt_5- | ABE_NNGRRT_21nt_5- | [′saCas9 | Splice Site |
| 14_011_+_31949411_saCas9 | 14_011 | ABE′] | |
| GuideDesign3_ABE_NNGRRT_21nt_5- | ABE_NNGRRT_21nt_5- | [′saCas9 | Splice Site |
| 14_011_+_31949411_saCas9 | 14_011 | ABE′] | |
| GuideDesign3_ABE_NNGRRT_21nt_5- | ABE_NNGRRT_21nt_5- | [′saCas9 | Splice Site |
| 14_011_+_31950042_saCas9 | 14_011 | ABE′] | |
| GuideDesign3_ABE_NNGRRT_21nt_5- | ABE_NNGRRT_21nt_5- | [′saCas9 | Splice Site |
| 14_011_+_31950042_saCas9 | 14_011 | ABE′] | |
| GuideDesign3_ABE_NNNRRT_21nt_5- | ABE_NNNRRT_21nt_5- | [′saCas9 | Splice Site |
| 14_014_−_31947176_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31947176_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31948067_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31948067_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31948499_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31948499_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31948942_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31948942_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31949545_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31949545_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31950135_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31950135_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31950387_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31950387_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31950925_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31950925_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31950928_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_−_31950928_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_+_31946366_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_+_31946366_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_+_31948817_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_+_31948817_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_+_31951334_saCas9 | 14_014 | KKH | |
| | | ABE′] | |
| GuideDesign3_ABE_NNNRRT_21nt_5- | ABE_NNNRRT_21nt_5- | [′saCas9 | Splice Site |
| 14_014_+_31951334_saCas9 | 14_014 | KKH | |
| | | ABE′] | |
| GuideDesign3_CBE_NNGRRT_21nt_3- | CBE_NNGRRT_21nt_3- | [′saCas9 | Splice Site |
| 12_012_−_31947927_saCas9 | 12_012 | CBE′] | |
| GuideDesign3_CBE_NNGRRT_21nt_3- | CBE_NNGRRT_21nt_3- | [′saCas9 | Splice Site |
| 12_012_−_31947927_saCas9 | 12_012 | CBE′] | |
| GuideDesign3_CBE_NNGRRT_21nt_3- | CBE_NNGRRT_21nt_3- | [′saCas9 | Splice Site |
| 12_012_−_31949397_saCas9 | 12_012 | CBE′] | |
| GuideDesign3_CBE_NNGRRT_21nt_3- | CBE_NNGRRT_21nt_3- | [′saCas9 | Splice Site |
| 12_012_−_31949397_saCas9 | 12_012 | CBE′] | |
| GuideDesign3_CBE_NNGRRT_21nt_3- | CBE_NNGRRT_21nt_3- | [′saCas9 | Splice Site |
| 12_012_−_31950031_saCas9 | 12_012 | CBE′] | |
| GuideDesign3_CBE_NNGRRT_21nt_3- | CBE_NNGRRT_21nt_3- | [′saCas9 | Splice Site |
| 12_012_−_31950031_saCas9 | 12_012 | CBE′] | |
| GuideDesign3_CBE_NNGRRT_21nt_3- | CBE_NNGRRT_21nt_3- | [′saCas9 | Splice Site |
| 12_012_−_31951856_saCas9 | 12_012 | CBE′] | |
| GuideDesign3_CBE_NNGRRT_21nt_3- | CBE_NNGRRT_21nt_3- | [′saCas9 | Splice Site |
| 12_012_−_31951856_saCas9 | 12_012 | CBE′] | |
| GuideDesign3_CBE_NNNRRT_21nt_3- | CBE_NNNRRT_21nt_3- | [′saCas9 | Splice Site |
| 12_015_−_31946263_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31946263_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31946354_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31946354_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31946985_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31946985_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31947322_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31947322_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31947719_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31947719_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31947724_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31947724_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31950269_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31950269_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31950599_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31950599_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31950922_saCas9 | 12_015 | KKH | |
| | | CBE′] | |
| GuideDesign3_CBE_NNNRRT_21nt_3- | CBE_NNNRRT_21nt_3- | [′saCas9 | Splice Site |
| 12_015_−_31950922_saCas9 | 12_015 | KKH | |
| | | CBE′] | |
| GuideDesign10_CBE_NGC_20nt_4- | CBE_NGC_20nt_4- | spCas9 | Stop Codon |
| 9_009_−_31947141_spCas9 | 9_009 | NGC | |
| | | CBE | |
| GuideDesign10_CBE_NGC_20nt_4- | CBE_NGC_20nt_4- | spCas9 | Stop Codon |
| 9_009_−_31948418_spCas9 | 9_009 | NGC | |
| | | CBE | |
| GuideDesign10_CBE_NGC_20nt_4- | CBE_NGC_20nt_4- | spCas9 | Stop Codon |
| 9_009_−_31948461_spCas9 | 9_009 | NGC | |
| | | CBE | |
| GuideDesign10_CBE_NGC_20nt_4- | CBE_NGC_20nt_4- | spCas9 | Stop Codon |
| 9_009_+_31946572_spCas9 | 9_009 | NGC | |
| | | CBE | |
| GuideDesign10_CBE_NGC_20nt_4- | CBE_NGC_20nt_4- | spCas9 | Stop Codon |
| 9_009_+_31947152_spCas9 | 9_009 | NGC | |
| | | CBE | |
| GuideDesign10_CBE_NGC_20nt_4- | CBE_NGC_20nt_4- | spCas9 | Stop Codon |
| 9_009_+_31947758_spCas9 | 9_009 | NGC | |
| | | CBE | |
| GuideDesign10_CBE_NGC_20nt_4- | CBE_NGC_20nt_4- | spCas9 | Stop Codon |
| 9_009_+_31948862_spCas9 | 9_009 | NGC | |
| | | CBE | |
| GuideDesign10_CBE_NGC_20nt_4- | CBE_NGC_20nt_4- | spCas9 | Stop Codon |
| 9_009_+_31950757_spCas9 | 9_009 | NGC | |
| | | CBE | |
| GuideDesign10_CBE_NGC_20nt_4- | CBE_NGC_20nt_4- | spCas9 | Stop Codon |
| 9_009_+_31950937_spCas9 | 9_009 | NGC | |
| | | CBE | |
| GuideDesign10_CBE_NGC_20nt_4- | CBE_NGC_20nt_4- | spCas9 | Stop Codon |
| 9_009_+_31951925_spCas9 | 9_009 | NGC | |
| | | CBE | |
| GuideDesign10_CBE_NGC_20nt_4- | CBE_NGC_20nt_4- | spCas9 | Stop Codon |
| 9_009_+_31951966_spCas9 | 9_009 | NGC | |
| | | CBE | |
| GuideDesign3_CBE_NNGRRT_21nt_3- | CBE_NNGRRT_21nt_3- | [′saCas9 | Stop Codon |
| 12_012_+_31951994_saCas9 | 12_012 | CBE′] | |
| GuideDesign3_CBE_NNNRRT_21nt_3- | CBE_NNNRRT_21nt_3- | [′saCas9 | Stop Codon |
| 12_015_−_31950102_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31946459_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31946513_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31946573_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31947137_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31949486_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31949493_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31950110_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31951909_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31951994_saCas9 | 12_015 | KKH | |
| | | CBE′] | |
| GuideDesign10_CBE_NGA_20nt_4- | CBE_NGA_20nt_4- | spCas9 | Stop Codon |
| 9_006_−_31946539_spCas9 | 9_006 | VRQR | |
| | | CBE | |
| GuideDesign10_CBE_NGA_20nt_4- | CBE_NGA_20nt_4- | spCas9 | Stop Codon |
| 9_006_−_31947033_spCas9 | 9_006 | VRQR | |
| | | CBE | |
| GuideDesign10_CBE_NGA_20nt_4- | CBE_NGA_20nt_4- | spCas9 | Stop Codon |
| 9_006_−_31947477_spCas9 | 9_006 | VRQR | |
| | | CBE | |
| GuideDesign10_CBE_NGA_20nt_4- | CBE_NGA_20nt_4- | spCas9 | Stop Codon |
| 9_006_−_31948907_spCas9 | 9_006 | VRQR | |
| | | CBE | |
| GuideDesign10_CBE_NGA_20nt_4- | CBE_NGA_20nt_4- | spCas9 | Stop Codon |
| 9_006_−_31950068_spCas9 | 9_006 | VRQR | |
| | | CBE | |
| GuideDesign10_CBE_NGA_20nt_4- | CBE_NGA_20nt_4- | spCas9 | Stop Codon |
| 9_006_−_31950070_spCas9 | 9_006 | VRQR | |
| | | CBE | |
| GuideDesign10_CBE_NGA_20nt_4- | CBE_NGA_20nt_4- | spCas9 | Stop Codon |
| 9_006_−_31951968_spCas9 | 9_006 | VRQR | |
| | | CBE | |
| GuideDesign10_CBE_NGA_20nt_4- | CBE_NGA_20nt_4- | spCas9 | Stop Codon |
| 9_006_−_31951972_spCas9 | 9_006 | VRQR | |
| | | CBE | |
| GuideDesign10_CBE_NGA_20nt_4- | CBE_NGA_20nt_4- | spCas9 | Stop Codon |
| 9_006_+_31946404_spCas9 | 9_006 | VRQR | |
| | | CBE | |
| GuideDesign10_CBE_NGA_20nt_4- | CBE_NGA_20nt_4- | spCas9 | Stop Codon |
| 9_006_+_31946568_spCas9 | 9_006 | VRQR | |
| | | CBE | |
| GuideDesign10_CBE_NGA_20nt_4- | CBE_NGA_20nt_4- | spCas9 | Stop Codon |
| 9_006_+_31947135_spCas9 | 9_006 | VRQR | |
| | | CBE | |
| GuideDesign10_CBE_NGA_20nt_4- | CBE_NGA_20nt_4- | spCas9 | Stop Codon |
| 9_006_+_31947139_spCas9 | 9_006 | VRQR | |
| | | CBE | |
| GuideDesign10_CBE_NGA_20nt_4- | CBE_NGA_20nt_4- | spCas9 | Stop Codon |
| 9_006_+_31947148_spCas9 | 9_006 | VRQR | |
| | | CBE | |
| GuideDesign10_CBE_NGA_20nt_4- | CBE_NGA_20nt_4- | spCas9 | Stop Codon |
| 9_006_+_31947161_spCas9 | 9_006 | VRQR | |
| | | CBE | |
| GuideDesign10_CBE_NGA_20nt_4- | CBE_NGA_20nt_4- | spCas9 | Stop Codon |
| 9_006_+_31947394_spCas9 | 9_006 | VRQR | |
| | | CBE | |
| GuideDesign10_CBE_NGA_20nt_4- | CBE_NGA_20nt_4- | spCas9 | Stop Codon |
| 9_006_+_31947460_spCas9 | 9_006 | VRQR | |
| | | CBE | |
| GuideDesign10_CBE_NGA_20nt_4- | CBE_NGA_20nt_4- | spCas9 | Stop Codon |
| 9_006_+_31947474_spCas9 | 9_006 | VRQR | |
| | | CBE | |
| GuideDesign10_CBE_NGA_20nt_4- | CBE_NGA_20nt_4- | spCas9 | Stop Codon |
| 9_006_+_31947942_spCas9 | 9_006 | VRQR | |
| | | CBE | |
| GuideDesign10_CBE_NGA_20nt_4- | CBE_NGA_20nt_4- | spCas9 | Stop Codon |
| 9_006_+_31948866_spCas9 | 9_006 | VRQR | |
| | | CBE | |
| GuideDesign10_CBE_NGA_20nt_4- | CBE_NGA_20nt_4- | spCas9 | Stop Codon |
| 9_006_+_31949541_spCas9 | 9_006 | VRQR | |
| | | CBE | |
| GuideDesign10_CBE_NGA_20nt_4- | CBE_NGA_20nt_4- | spCas9 | Stop Codon |
| 9_006_+_31949543_spCas9 | 9_006 | VRQR | |
| | | CBE | |
| GuideDesign10_CBE_NGA_20nt_4- | CBE_NGA_20nt_4- | spCas9 | Stop Codon |
| 9_006_+_31950113_spCas9 | 9_006 | VRQR | |
| | | CBE | |
| GuideDesign10_CBE_NGA_20nt_4- | CBE_NGA_20nt_4- | spCas9 | Stop Codon |
| 9_006_+_31950755_spCas9 | 9_006 | VRQR | |
| | | CBE | |
| GuideDesign10_CBE_NGA_20nt_4- | CBE_NGA_20nt_4- | spCas9 | Stop Codon |
| 9_006_+_31950927_spCas9 | 9_006 | VRQR | |
| | | CBE | |
| GuideDesign10_CBE_NGA_20nt_4- | CBE_NGA_20nt_4- | spCas9 | Stop Codon |
| 9_006_+_31951361_spCas9 | 9_006 | VRQR | |
| | | CBE | |
| GuideDesign10_CBE_NGA_20nt_4- | CBE_NGA_20nt_4- | spCas9 | Stop Codon |
| 9_006_+_31951969_spCas9 | 9_006 | VRQR | |
| | | CBE | |
| GuideDesign10_CBE_NGG_20nt_4- | CBE_NGG_20nt_4- | spCas9 | Stop Codon |
| 9_003_−_31947142_spCas9 | 9_003 | CBE | |
| GuideDesign10_CBE_NGG_20nt_4- | CBE_NGG_20nt_4- | spCas9 | Stop Codon |
| 9_003_−_31948419_spCas9 | 9_003 | CBE | |
| GuideDesign10_CBE_NGG_20nt_4- | CBE_NGG_20nt_4- | spCas9 | Stop Codon |
| 9_003_−_31948908_spCas9 | 9_003 | CBE | |
| GuideDesign10_CBE_NGG_20nt_4- | CBE_NGG_20nt_4- | spCas9 | Stop Codon |
| 9_003_−_31948909_spCas9 | 9_003 | CBE | |
| GuideDesign10_CBE_NGG_20nt_4- | CBE_NGG_20nt_4- | spCas9 | Stop Codon |
| 9_003_−_31950110_spCas9 | 9_003 | CBE | |
| GuideDesign10_CBE_NGG_20nt_4- | CBE_NGG_20nt_4- | spCas9 | Stop Codon |
| 9_003_+_31946448_spCas9 | 9_003 | CBE | |
| GuideDesign10_CBE_NGG_20nt_4- | CBE_NGG_20nt_4- | spCas9 | Stop Codon |
| 9_003_+_31946449_spCas9 | 9_003 | CBE | |
| GuideDesign10_CBE_NGG_20nt_4- | CBE_NGG_20nt_4- | spCas9 | Stop Codon |
| 9_003_+_31946460_spCas9 | 9_003 | CBE | |
| GuideDesign10_CBE_NGG_20nt_4- | CBE_NGG_20nt_4- | spCas9 | Stop Codon |
| 9_003_+_31946567_spCas9 | 9_003 | CBE | |
| GuideDesign10_CBE_NGG_20nt_4- | CBE_NGG_20nt_4- | spCas9 | Stop Codon |
| 9_003_+_31946571_spCas9 | 9_003 | CBE | |
| GuideDesign10_CBE_NGG_20nt_4- | CBE_NGG_20nt_4- | spCas9 | Stop Codon |
| 9_003_+_31947138_spCas9 | 9_003 | CBE | |
| GuideDesign10_CBE_NGG_20nt_4- | CBE_NGG_20nt_4- | spCas9 | Stop Codon |
| 9_003_+_31947459_spCas9 | 9_003 | CBE | |
| GuideDesign10_CBE_NGG_20nt_4- | CBE_NGG_20nt_4- | spCas9 | Stop Codon |
| 9_003_+_31947463_spCas9 | 9_003 | CBE | |
| GuideDesign10_CBE_NGG_20nt_4- | CBE_NGG_20nt_4- | spCas9 | Stop Codon |
| 9_003_+_31947466_spCas9 | 9_003 | CBE | |
| GuideDesign10_CBE_NGG_20nt_4- | CBE_NGG_20nt_4- | spCas9 | Stop Codon |
| 9_003_+_31947473_spCas9 | 9_003 | CBE | |
| GuideDesign10_CBE_NGG_20nt_4- | CBE_NGG_20nt_4- | spCas9 | Stop Codon |
| 9_003_+_31949539_spCas9 | 9_003 | CBE | |
| GuideDesign10_CBE_NGG_20nt_4- | CBE_NGG_20nt_4- | spCas9 | Stop Codon |
| 9_003_+_31949540_spCas9 | 9_003 | CBE | |
| GuideDesign10_CBE_NGG_20nt_4- | CBE_NGG_20nt_4- | spCas9 | Stop Codon |
| 9_003_+_31950936_spCas9 | 9_003 | CBE | |
| GuideDesign10_CBE_NGG_20nt_4- | CBE_NGG_20nt_4- | spCas9 | Stop Codon |
| 9_003_+_31951965_spCas9 | 9_003 | CBE | |
| GuideDesign10_CBE_NGG_20nt_4- | CBE_NGG_20nt_4- | spCas9 | Stop Codon |
| 9_003_+_31951996_spCas9 | 9_003 | CBE | |
| GuideDesign10_CBE_NGG_20nt_4- | CBE_NGG_20nt_4- | spCas9 | Stop Codon |
| 9_003_+_31951997_spCas9 | 9_003 | CBE | |
| GuideDesign3_CBE_NNGRRT_21nt_3- | CBE_NNGRRT_21nt_3- | [′saCas9 | Stop Codon |
| 12_012_+_31946459_saCas9 | 12_012 | CBE′] | |
| GuideDesign3_CBE_NNGRRT_21nt_3- | CBE_NNGRRT_21nt_3- | [′saCas9 | Stop Codon |
| 12_012_+_31946459_saCas9 | 12_012 | CBE′] | |
| GuideDesign3_CBE_NNGRRT_21nt_3- | CBE_NNGRRT_21nt_3- | [′saCas9 | Stop Codon |
| 12_012_+_31946573_saCas9 | 12_012 | CBE′] | |
| GuideDesign3_CBE_NNGRRT_21nt_3- | CBE_NNGRRT_21nt_3- | [′saCas9 | Stop Codon |
| 12_012_+_31946573_saCas9 | 12_012 | CBE′] | |
| GuideDesign3_CBE_NNGRRT_21nt_3- | CBE_NNGRRT_21nt_3- | [′saCas9 | Stop Codon |
| 12_012_+_31947137_saCas9 | 12_012 | CBE′] | |
| GuideDesign3_CBE_NNGRRT_21nt_3- | CBE_NNGRRT_21nt_3- | [′saCas9 | Stop Codon |
| 12_012_+_31947137_saCas9 | 12_012 | CBE′] | |
| GuideDesign3_CBE_NNGRRT_21nt_3- | CBE_NNGRRT_21nt_3- | [′saCas9 | Stop Codon |
| 12_012_+_31949493_saCas9 | 12_012 | CBE′] | |
| GuideDesign3_CBE_NNGRRT_21nt_3- | CBE_NNGRRT_21nt_3- | [′saCas9 | Stop Codon |
| 12_012_+_31949493_saCas9 | 12_012 | CBE′] | |
| GuideDesign3_CBE_NNNRRT_21nt_3- | CBE_NNNRRT_21nt_3- | [′saCas9 | Stop Codon |
| 12_015_−_31946530_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_−_31946530_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_−_31946538_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_−_31946538_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_−_31947030_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_−_31947030_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_−_31947137_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_−_31947137_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_−_31950106_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_−_31950106_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_−_31950109_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_−_31950109_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_−_31951965_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_−_31951965_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31947132_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31947132_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31947151_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31947151_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31947460_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31947460_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31947939_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31947939_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31948478_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31948478_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31948863_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31948863_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31949540_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31949540_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31950116_saCas9 | 12_015 | KKH | |
| | | CBE′] | |
| GuideDesign3_CBE_NNNRRT_21nt_3- | CBE_NNNRRT_21nt_3- | [′saCas9 | Stop Codon |
| 12_015_+_31950116_saCas9 | 12_015 | KKH | |
| | | CBE′] | |
| Guide_polynucleotide | Target_sequence | SEQ | |
| name | | ID | seque |
| | | NO | nce |
| GuideDesign10_CBE_N | CTTCTCACCTCCGTTGT | 2055 | AGA |
| GA_20nt_4-9_006_- | CACAGA | | |
| 31947177_spCas9 | | | |
| GuideDesign10_CBE_N | CTGACCATCAGTCATGA | 2056 | TGA |
| GA_20nt_4-9_006_- | GGATGA | | |
| 31948943_spCas9 | | | |
| GuideDesign10_CBE_N | CTCCCTACCGATCATTT | 2057 | AGA |
| GA_20nt_4-9_006_- | GGTAGA | | |
| 31949542_spCas9 | | | |
| GuideDesign10_CBE_N | TTACCTACGCTGACCTT | 2058 | TGA |
| GA_20nt_4-9_006_- | GATTGA | | |
| 31950384_spCas9 | | | |
| GuideDesign10_CBE_N | ATCCTTACCTACGCTGA | 2059 | TGA |
| GA_20nt_4-9_006_- | CCTTGA | | |
| 31950388_spCas9 | | | |
| GuideDesign10_CBE_N | TCTCACCTTATCCCCAT | 2060 | TGA |
| GA_20nt_4-9_006_- | TCTTGA | | |
| 31951227_spCas9 | | | |
| GuideDesign10_CBE_N | TCGCTTACCTCCAGACA | 2061 | GGC |
| GC_20nt_4-9_009_- | AGAGGC | | |
| 31946270_spCas9 | | | |
| GuideDesign10_CBE_N | TCACCTCCGTTGTCACA | 2062 | CGC |
| GC_20nt_4-9_009_- | GATCGC | | |
| 31947173_spCas9 | | | |
| GuideDesign10_CBE_N | CTTACAATGACTGAGAT | 2063 | GGC |
| GC_20nt_4-9_009_- | CTTGGC | | |
| 31950129_spCas9 | | | |
| GuideDesign10_CBE_N | AACCTGCAGAGGCAGTG | 2064 | TGC |
| GC_20nt_4-9_009_- | GGGTGC | | |
| 31951854_spCas9 | | | |
| GuideDesign10_CBE_N | CTCGCTTACCTCCAGAC | 2065 | AGG |
| GG_20nt_4-9_003_- | AAGAGG | | |
| 31946271_spCas9 | | | |
| GuideDesign10_CBE_N | CCTTCTGACCATCAGTC | 2066 | AGG |
| GG_20nt_4-9_003_- | ATGAGG | | |
| 31948947_spCas9 | | | |
| GuideDesign10_CBE_N | TCACCCAGATAATCCTC | 2067 | TGG |
| GG_20nt_4-9_003_- | CCTTGG | | |
| 31949325_spCas9 | | | |
| GuideDesign10_CBE_N | GCTTACAATGACTGAGA | 2068 | TGG |
| GG_20nt_4-9_003_- | TCTTGG | | |
| 31950130_spCas9 | | | |
| GuideDesign10_CBE_N | CTTACTTTGTTGCTGGC | 2069 | TGG |
| GG_20nt_4-9_003_- | AAGTGG | | |
| 31950926_spCas9 | | | |
| GuideDesign10_CBE_N | TCTCACCTCTGCAAGTA | 2070 | GGG |
| GG_20nt_4-9_003_- | TTGGGG | | |
| 31951456_spCas9 | | | |
| GuideDesign3_ABE_N | CCCTCGCTTACCTCCAG | 2071 | AGA |
| GA_20nt_3-12_019_- | ACAAGA | | |
| 31946273_spCas9 | | | |
| GuideDesign3_ABE_N | CTTCTCACCTCCGTTGT | 2072 | AGA |
| GA_20nt_3-12_019_- | CACAGA | | |
| 31947177_spCas9 | | | |
| GuideDesign3_ABE_N | GGTCAAAGGTCACCTTG | 2073 | GGA |
| GA_20nt_3-12_019_- | GCAGGA | | |
| 31947511_spCas9 | | | |
| GuideDesign3_ABE_N | TTCCACCTTCTCAATTA | 2074 | TGA |
| GA_20nt_3-12_019_- | AGTTGA | | |
| 31948064_spCas9 | | | |
| GuideDesign3_ABE_N | TAACCTCTGACCTTCAT | 2075 | TGA |
| GA_20nt_3-12_019_- | AATTGA | | |
| 31948500_spCas9 | | | |
| GuideDesign3_ABE_N | CTGACCATCAGTCATGA | 2076 | TGA |
| GA_20nt_3-12_019_- | GGATGA | | |
| 31948943_spCas9 | | | |
| GuideDesign3_ABE_N | CTTCTGACCATCAGTCA | 2077 | GGA |
| GA_20nt_3-12_019_- | TGAGGA | | |
| 31948946_spCas9 | | | |
| GuideDesign3_ABE_N | TCCCTTCTGACCATCAG | 2078 | TGA |
| GA_20nt_3-12_019_- | TCATGA | | |
| 31948949_spCas9 | | | |
| GuideDesign3_ABE_N | CTCCCTACCGATCATTT | 2079 | AGA |
| GA_20nt_3-12_019_- | GGTAGA | | |
| 31949542_spCas9 | | | |
| GuideDesign3_ABE_N | TTCTGTGCTTACAATGA | 2080 | AGA |
| GA_20nt_3-12_019_- | CTGAGA | | |
| 31950136_spCas9 | | | |
| GuideDesign3_ABE_N | TTACCTACGCTGACCTT | 2081 | TGA |
| GA_20nt_3-12_019_- | GATTGA | | |
| 31950384_spCas9 | | | |
| GuideDesign3_ABE_N | ATCCTTACCTACGCTGA | 2082 | TGA |
| GA_20nt_3-12_019_- | CCTTGA | | |
| 31950388_spCas9 | | | |
| GuideDesign3_ABE_N | TCTCACCTTATCCCCAT | 2083 | TGA |
| GA_20nt_3-12_019_- | TCTTGA | | |
| 31951227_spCas9 | | | |
| GuideDesign3_ABE_N | GAGGACTCACTTGAATG | 2084 | CGA |
| GA_20nt_3-12_019_- | AAACGA | | |
| 31951591_spCas9 | | | |
| GuideDesign3_ABE_N | TCAAAGCAATCCACTGT | 2085 | AGA |
| GA_20nt_3- | CCAAGA | | |
| 12_019_+_31947000_sp | | | |
| Cas9 | | | |
| GuideDesign3_ABE_N | CTCCCAGACTCCTTCAT | 2086 | CGA |
| GA_20nt_3- | GTACGA | | |
| 12_019_+_31947734_sp | | | |
| Cas9 | | | |
| GuideDesign3_ABE_N | CTGGCACCCAGGGGAAC | 2087 | AGA |
| GA_20nt_3- | AACAGA | | |
| 12_019_+_31947933_sp | | | |
| Cas9 | | | |
| GuideDesign3_ABE_N | CCCAGGGGAACAACAGA | 2088 | GGA |
| GA_20nt_3- | AGCGGA | | |
| 12_019_+_31947939_sp | | | |
| Cas9 | | | |
| GuideDesign3_ABE_N | ACAGGTGGCAAGTTATG | 2089 | TGA |
| GA_20nt_3- | GTGTGA | | |
| 12_019_+_31948369_sp | | | |
| Cas9 | | | |
| GuideDesign3_ABE_N | CCTCTCCACAGACCACA | 2090 | TGA |
| GA_20nt_3- | AGTTGA | | |
| 12_019_+_31948818_sp | | | |
| Cas9 | | | |
| GuideDesign3_ABE_N | GCCAGATGAAAGCCAGT | 2091 | TGA |
| GA_20nt_3- | CTCTGA | | |
| 12_019_+_31950044_sp | | | |
| Cas9 | | | |
| GuideDesign3_ABE_N | GTCCCCAGCGCCCTTCA | 2092 | GGA |
| GA_20nt_3- | AAGGGA | | |
| 12_019_+_31950277_sp | | | |
| Cas9 | | | |
| GuideDesign3_ABE_N | ACTTCAGGAGGGGAGAA | 2093 | GGA |
| GA_20nt_3- | GCGGGA | | |
| 12_019_+_31950611_sp | | | |
| Cas9 | | | |
| GuideDesign3_ABE_N | CCCCTACAGAAAGGCAG | 2094 | TGA |
| GA_20nt_3- | CTGTGA | | |
| 12_019_+_31951331_sp | | | |
| Cas9 | | | |
| GuideDesign3_ABE_N | CCTACAGAAAGGCAGCT | 2095 | AGA |
| GA_20nt_3- | GTGAGA | | |
| 12_019_+_31951333_sp | | | |
| Cas9 | | | |
| GuideDesign3_ABE_N | TACAGAAAGGCAGCTGT | 2096 | AGA |
| GA_20nt_3- | GAGAGA | | |
| 12_019_+_31951335_sp | | | |
| Cas9 | | | |
| GuideDesign3_ABE_N | TTCCACCTTCTCAATTA | 2097 | TGA |
| GA_20nt_3-9_005_- | AGTTGA | | |
| 31948064_spCas9 | | | |
| GuideDesign3_ABE_N | CTTCTGACCATCAGTCA | 2098 | GGA |
| GA_20nt_3-9_005_- | TGAGGA | | |
| 31948946_spCas9 | | | |
| GuideDesign3_ABE_N | ACTTCAGGAGGGGAGAA | 2099 | GGA |
| GA_20nt_3- | GCGGGA | | |
| 9_005_+_31950611_spC | | | |
| as9 | | | |
| GuideDesign3_ABE_N | CCCCTACAGAAAGGCAG | 2100 | TGA |
| GA_20nt_3- | CTGTGA | | |
| 9_005_+_31951331_spC | | | |
| as9 | | | |
| GuideDesign3_ABE_N | CCTACAGAAAGGCAGCT | 2101 | AGA |
| GA_20nt_3- | GTGAGA | | |
| 9_005_+_31951333_spC | | | |
| as9 | | | |
| GuideDesign3_ABE_N | TCGCTTACCTCCAGACA | 2102 | GGC |
| GC_20nt_3-12_020_- | AGAGGC | | |
| 31946270_spCas9 | | | |
| GuideDesign3_ABE_N | GCCCTCAAACCTCTGCA | 2103 | TGC |
| GC_20nt_3-12_020_- | CTCTGC | | |
| 31946593_spCas9 | | | |
| GuideDesign3_ABE_N | TCACCTCCGTTGTCACA | 2104 | CGC |
| GC_20nt_3-12_020_- | GATCGC | | |
| 31947173_spCas9 | | | |
| GuideDesign3_ABE_N | AAAGGTCACCTTGGCAG | 2105 | GGC |
| GC_20nt_3-12_020_- | GAAGGC | | |
| 31947507_spCas9 | | | |
| GuideDesign3_ABE_N | CTGTCTTCAAACCTGGG | 2106 | TGC |
| GC_20nt_3-12_020_- | CCGTGC | | |
| 31947832_spCas9 | | | |
| GuideDesign3_ABE_N | CTTACAATGACTGAGAT | 2107 | GGC |
| GC_20nt_3-12_020_- | CTTGGC | | |
| 31950129_spCas9 | | | |
| GuideDesign3_ABE_N | ACGCTCTCACCTGATAG | 2108 | GGC |
| GC_20nt_3-12_020_- | TCTGGC | | |
| 31950759_spCas9 | | | |
| GuideDesign3_ABE_N | GTATGTCTTACTTTGTT | 2109 | GGC |
| GC_20nt_3-12_020_- | GCTGGC | | |
| 31950932_spCas9 | | | |
| GuideDesign3_ABE_N | ATACCTCTGCAGCGGGG | 2110 | TGC |
| GC_20nt_3- | TACTGC | | |
| 12_020_+_31947335_sp | | | |
| Cas9 | | | |
| GuideDesign3_ABE_N | GCACCCAGGGGAACAAC | 2111 | AGC |
| GC_20nt_3- | AGAAGC | | |
| 12_020_+_31947936_sp | | | |
| Cas9 | | | |
| GuideDesign3_ABE_N | CTGCAGGATTGCACAAC | 2112 | GGC |
| GC_20nt_3- | ATGGGC | | |
| 12_020_+_31949236_sp | | | |
| Cas9 | | | |
| GuideDesign3_ABE_N | TCCTACTTCAGGAGGGG | 2113 | AGC |
| GC_20nt_3- | AGAAGC | | |
| 12_020_+_31950607_sp | | | |
| Cas9 | | | |
| GuideDesign3_ABE_N | ATAGGCCCATTTGTCTC | 2114 | TGC |
| GC_20nt_3- | CCCTGC | | |
| 12_020_+_31950863_sp | | | |
| Cas9 | | | |
| GuideDesign3_ABE_N | TATTCGTCCAGAGGAAG | 2115 | TGC |
| GC_20nt_3- | AGCTGC | | |
| 12_020_+_31951132_sp | | | |
| Cas9 | | | |
| GuideDesign3_ABE_N | CCAGAGGAAGAGCTGCT | 2116 | TGC |
| GC_20nt_3- | CCCTGC | | |
| 12_020_+_31951139_sp | | | |
| Cas9 | | | |
| GuideDesign3_ABE_N | CTCTCCTTGCAGGTGAT | 2117 | GGC |
| GC_20nt_3- | TCTGGC | | |
| 12_020_+_31951542_sp | | | |
| Cas9 | | | |
| GuideDesign3_ABE_N | TCCTTGCAGGTGATTCT | 2118 | GGC |
| GC_20nt_3- | GGCGGC | | |
| 12_020_+_31951545_sp | | | |
| Cas9 | | | |
| GuideDesign3_ABE_N | CTCTGCAGGTTGGTGTA | 2119 | AGC |
| GC_20nt_3- | ATCAGC | | |
| 12_020_+_31951866_sp | | | |
| Cas9 | | | |
| GuideDesign3_ABE_N | GCCCTCAAACCTCTGCA | 2120 | TGC |
| GC_20nt_3-9_008_- | CTCTGC | | |
| 31946593_spCas9 | | | |
| GuideDesign3_ABE_N | AAAGGTCACCTTGGCAG | 2121 | GGC |
| GC_20nt_3-9_008_- | GAAGGC | | |
| 31947507_spCas9 | | | |
| GuideDesign3_ABE_N | GCACCCAGGGGAACAAC | 2122 | AGC |
| GC_20nt_3- | AGAAGC | | |
| 9_008_+_31947936_spC | | | |
| as9 | | | |
| GuideDesign3_ABE_N | CCAGAGGAAGAGCTGCT | 2123 | TGC |
| GC_20nt_3- | CCCTGC | | |
| 9_008_+_31951139_spC | | | |
| as9 | | | |
| GuideDesign3_ABE_N | TCCTTGCAGGTGATTCT | 2124 | GGC |
| GC_20nt_3- | GGCGGC | | |
| 9_008_+_31951545_spC | | | |
| as9 | | | |
| GuideDesign3_ABE_N | CTCGCTTACCTCCAGAC | 2125 | AGG |
| GG_20nt_3-12_018_- | AAGAGG | | |
| 31946271_spCas9 | | | |
| GuideDesign3_ABE_N | CAAAGGTCACCTTGGCA | 2126 | AGG |
| GG_20nt_3-12_018_- | GGAAGG | | |
| 31947508_spCas9 | | | |
| GuideDesign3_ABE_N | CCTTCTGACCATCAGTC | 2127 | AGG |
| GG_20nt_3-12_018_- | ATGAGG | | |
| 31948947_spCas9 | | | |
| GuideDesign3_ABE_N | TCACCCAGATAATCCTC | 2128 | TGG |
| GG_20nt_3-12_018_- | CCTTGG | | |
| 31949325_spCas9 | | | |
| GuideDesign3_ABE_N | GTATCTCCCTACCGATC | 2129 | TGG |
| GG_20nt_3-12_018_- | ATTTGG | | |
| 31949546_spCas9 | | | |
| GuideDesign3_ABE_N | GCTTACAATGACTGAGA | 2130 | TGG |
| GG_20nt_3-12_018_- | TCTTGG | | |
| 31950130_spCas9 | | | |
| GuideDesign3_ABE_N | GACGCTCTCACCTGATA | 2131 | TGG |
| GG_20nt_3-12_018_- | GTCTGG | | |
| 31950760_spCas9 | | | |
| GuideDesign3_ABE_N | CTTACTTTGTTGCTGGC | 2132 | TGG |
| GG_20nt_3-12_018_- | AAGTGG | | |
| 31950926_spCas9 | | | |
| GuideDesign3_ABE_N | AGTATGTCTTACTTTGT | 2133 | TGG |
| GG_20nt_3-12_018_- | TGCTGG | | |
| 31950933_spCas9 | | | |
| GuideDesign3_ABE_N | TCTCACCTCTGCAAGTA | 2134 | GGG |
| GG_20nt_3-12_018_- | TTGGGG | | |
| 31951456_spCas9 | | | |
| GuideDesign3_ABE_N | CTCTCACCTCTGCAAGT | 2135 | GGG |
| GG_20nt_3-12_018_- | ATTGGG | | |
| 31951457_spCas9 | | | |
| GuideDesign3_ABE_N | TCTCTCACCTCTGCAAG | 2136 | TGG |
| GG_20nt_3-12_018_- | TATTGG | | |
| 31951458_spCas9 | | | |
| GuideDesign3_ABE_N | ACCCAGGGGAACAACAG | 2137 | CGG |
| GG_20nt_3- | AAGCGG | | |
| 12_018_+_31947938_sp | | | |
| Cas9 | | | |
| GuideDesign3_ABE_N | CACCCACAGGTGGCAAG | 2138 | TGG |
| GG_20nt_3- | TTATGG | | |
| 12_018_+_31948364_sp | | | |
| Cas9 | | | |
| GuideDesign3_ABE_N | ACAGACCACAAGTTGAA | 2139 | AGG |
| GG_20nt_3- | GTCAGG | | |
| 12_018_+_31948825_sp | | | |
| Cas9 | | | |
| GuideDesign3_ABE_N | TTCTGCAGGATTGCACA | 2140 | TGG |
| GG_20nt_3- | ACATGG | | |
| 12_018_+_31949234_sp | | | |
| Cas9 | | | |
| GuideDesign3_ABE_N | TCTGCAGGATTGCACAA | 2141 | GGG |
| GG_20nt_3- | CATGGG | | |
| 12_018_+_31949235_sp | | | |
| Cas9 | | | |
| GuideDesign3_ABE_N | GCAGGATTGCACAACAT | 2142 | CGG |
| GG_20nt_3- | GGGCGG | | |
| 12_018_+_31949238_sp | | | |
| Cas9 | | | |
| GuideDesign3_ABE_N | CCCTCAGATGTCTATGT | 2143 | TGG |
| GG_20nt_3- | GTTTGG | | |
| 12_018_+_31949412_sp | | | |
| Cas9 | | | |
| GuideDesign3_ABE_N | CCTCAGATGTCTATGTG | 2144 | GGG |
| GG_20nt_3- | TTTGGG | | |
| 12_018_+_31949413_sp | | | |
| Cas9 | | | |
| GuideDesign3_ABE_N | CTCAGATGTCTATGTGT | 2145 | GGG |
| GG_20nt_3- | TTGGGG | | |
| 12_018_+_31949414_sp | | | |
| Cas9 | | | |
| GuideDesign3_ABE_N | GTGTCCCCAGCGCCCTT | 2146 | AGG |
| GG_20nt_3- | CAAAGG | | |
| 12_018_+_31950275_sp | | | |
| Cas9 | | | |
| GuideDesign3_ABE_N | TGTCCCCAGCGCCCTTC | 2147 | GGG |
| GG_20nt_3- | AAAGGG | | |
| 12_018_+_31950276_sp | | | |
| Cas9 | | | |
| GuideDesign3_ABE_N | CTACTTCAGGAGGGGAG | 2148 | CGG |
| GG_20nt_3- | AAGCGG | | |
| 12_018_+_31950609_sp | | | |
| Cas9 | | | |
| GuideDesign3_ABE_N | TACTTCAGGAGGGGAGA | 2149 | GGG |
| GG_20nt_3- | AGCGGG | | |
| 12_018_+_31950610_sp | | | |
| Cas9 | | | |
| GuideDesign3_ABE_N | CCTCTCCTTGCAGGTGA | 2150 | TGG |
| GG_20nt_3- | TTCTGG | | |
| 12_018_+_31951541_sp | | | |
| Cas9 | | | |
| GuideDesign3_ABE_N | CTCCTTGCAGGTGATTC | 2151 | CGG |
| GG_20nt_3- | TGGCGG | | |
| 12_018_+_31951544_sp | | | |
| Cas9 | | | |
| GuideDesign3_ABE_N | TGCAGGTTGGTGTAATC | 2152 | TGG |
| GG_20nt_3- | AGCTGG | | |
| 12_018_+_31951869_sp | | | |
| Cas9 | | | |
| GuideDesign3_ABE_N | GCAGGTTGGTGTAATCA | 2153 | GGG |
| GG_20nt_3- | GCTGGG | | |
| 12_018_+_31951870_sp | | | |
| Cas9 | | | |
| GuideDesign3_ABE_N | CTCTCACCTCTGCAAGT | 2154 | GGG |
| GG_20nt_3-9_002_- | ATTGGG | | |
| 31951457_spCas9 | | | |
| GuideDesign3_ABE_N | TCTCTCACCTCTGCAAG | 2155 | TGG |
| GG_20nt_3-9_002_- | TATTGG | | |
| 31951458_spCas9 | | | |
| GuideDesign3_ABE_N | ACCCAGGGGAACAACAG | 2156 | CGG |
| GG_20nt_3- | AAGCGG | | |
| 9_002_+_31947938_spC | | | |
| as9 | | | |
| GuideDesign3_ABE_N | TTCTGCAGGATTGCACA | 2157 | TGG |
| GG_20nt_3- | ACATGG | | |
| 9_002_+_31949234_spC | | | |
| as9 | | | |
| GuideDesign3_ABE_N | TCCCTTCTGACCATCAG | 2158 | GAGG |
| NNRRT_21nt_5- | TCATGAGGAT | | AT |
| 14_014_- | | | |
| 31948945_saCas9 | | | |
| GuideDesign3_ABE_N | ACTCACCCAGATAATCC | 2159 | TTGG |
| NNRRT_21nt_5- | TCCCTTGGGT | | GT |
| 14_014_- | | | |
| 31949323_saCas9 | | | |
| GuideDesign3_ABE_N | CCTTACCTACGCTGACC | 2160 | TTGA |
| NNRRT_21nt_5- | TTGATTGAGT | | GT |
| 14_014_- | | | |
| 31950382_saCas9 | | | |
| GuideDesign3_ABE_N | GTTTCTCACCTTATCCC | 2161 | CTTG |
| NNRRT_21nt_5- | CATTCTTGAT | | AT |
| 14_014_- | | | |
| 31951226_saCas9 | | | |
| GuideDesign3_ABE_N | TTCTCTCACCTCTGCAA | 2162 | TGGG |
| NNRRT_21nt_5- | GTATTGGGGT | | GT |
| 14_014_- | | | |
| 31951455_saCas9 | | | |
| GuideDesign3_ABE_N | CCTCACCCACAGGTGGC | 2163 | TATG |
| NNRRT_21nt_5- | AAGTTATGGT | | GT |
| 14_014_+_31948361_sa | | | |
| Cas9 | | | |
| GuideDesign3_ABE_N | TCCCTCAGATGTCTATG | 2164 | TGGG |
| NNRRT_21nt_5- | TGTTTGGGGT | | GT |
| 14_014_+_31949411_sa | | | |
| Cas9 | | | |
| GuideDesign3_ABE_N | CTCCTTCTCTGCCAGAT | 2165 | GCCA |
| NNRRT_21nt_5- | GAAAGCCAGT | | GT |
| 14_014_+_31950034_sa | | | |
| Cas9 | | | |
| GuideDesign3_ABE_N | CTGCCAGATGAAAGCCA | 2166 | CTGA |
| NNRRT_21nt_5- | GTCTCTGAGT | | GT |
| 14_014_+_31950042_sa | | | |
| Cas9 | | | |
| GuideDesign3_ABE_VT | ATTGCCCTCAAACCTCT | 2167 | ATTG |
| TN_22nt_5-9_017_- | GCACTCTGC | | |
| 31946593_cas12b | | | |
| GuideDesign3_ABE_VT | ATTCTGTGCTTACAATG | 2168 | ATTC |
| TN_22nt_5-9_017_- | ACTGAGATC | | |
| 31950134_cas12b | | | |
| GuideDesign3_ABE_VT | GTTGCATCCTTACCTAC | 2169 | GTTG |
| TN_22nt_5-9_017_- | GCTGACCTT | | |
| 31950390_cas12b | | | |
| GuideDesign3_ABE_VT | ATTCTCTCACCTCTGCA | 2170 | ATTC |
| TN_22nt_5-9_017_- | AGTATTGGG | | |
| 31951457_cas12b | | | |
| GuideDesign3_ABE_VT | CTTTCTCTTCAGGTGTG | 2171 | CTTT |
| TN_22nt_5- | ACCACCACT | | |
| 9_017_+_31946360_cas | | | |
| 12b | | | |
| GuideDesign3_ABE_VT | CTTCTCAAAGCAATCCA | 2172 | CTTC |
| TN_22nt_5- | CTGTCCAAG | | |
| 9_017_+_31946996_cas | | | |
| 12b | | | |
| GuideDesign3_ABE_VT | CTTCTCTGCCAGATGAA | 2173 | CTTC |
| TN_22nt_5- | AGCCAGTCT | | |
| 9_017_+_31950037_cas | | | |
| 12b | | | |
| GuideDesign3_ABE_VT | GTTGTGTCCCCAGCGCC | 2174 | GTTG |
| TN_22nt_5- | CTTCAAAGG | | |
| 9_017_+_31950272_cas | | | |
| 12b | | | |
| GuideDesign3_ABE_VT | CTTGTCCTTTATAGGCC | 2175 | CTTG |
| TN_22nt_5- | CATTTGTCT | | |
| 9_017_+_31950853_cas | | | |
| 12b | | | |
| GuideDesign3_ABE_VT | ATTCGTCCAGAGGAAGA | 2176 | ATTC |
| TN_22nt_5- | GCTGCTCCC | | |
| 9_017_+_31951133_cas | | | |
| 12b | | | |
| GuideDesign3_CBE_N | TCCCTTCTGACCATCAG | 2177 | GAGG |
| NGRRT_21nt_3- | TCATGAGGAT | | AT |
| 12_012_- | | | |
| 31948945_saCas9 | | | |
| GuideDesign3_CBE_N | ACTCACCCAGATAATCC | 2178 | TTGG |
| NGRRT_21nt_3- | TCCCTTGGGT | | GT |
| 12_012_- | | | |
| 31949323_saCas9 | | | |
| GuideDesign3_CBE_N | CCTTACCTACGCTGACC | 2179 | TTGA |
| NGRRT_21nt_3- | TTGATTGAGT | | GT |
| 12_012_- | | | |
| 31950382_saCas9 | | | |
| GuideDesign3_CBE_N | TTCTCTCACCTCTGCAA | 2180 | TGGG |
| NGRRT_21nt_3- | GTATTGGGGT | | GT |
| 12_012_- | | | |
| 31951455_saCas9 | | | |
| GuideDesign3_CBE_N | ATGCTTCTCACCTCCGT | 2181 | ACAG |
| NNRRT_21nt_3- | TGTCACAGAT | | AT |
| 12_015_- | | | |
| 31947176_saCas9 | | | |
| GuideDesign3_CBE_N | TGTTGTTCCCCTGGGTG | 2182 | GAGA |
| NNRRT_21nt_3- | CCAGGAGAGT | | GT |
| 12_015_- | | | |
| 31947927_saCas9 | | | |
| GuideDesign3_CBE_N | CTTCTGACCATCAGTCA | 2183 | GATG |
| NNRRT_21nt_3- | TGAGGATGAT | | AT |
| 12_015_- | | | |
| 31948942_saCas9 | | | |
| GuideDesign3_CBE_N | TCCCTTCTGACCATCAG | 2184 | GAGG |
| NNRRT_21nt_3- | TCATGAGGAT | | AT |
| 12_015_- | | | |
| 31948945_saCas9 | | | |
| GuideDesign3_CBE_N | ACTCACCCAGATAATCC | 2185 | TTGG |
| NNRRT_21nt_3- | TCCCTTGGGT | | GT |
| 12_015_- | | | |
| 31949323_saCas9 | | | |
| GuideDesign3_CBE_N | GACATCTGAGGGATAAA | 2186 | AAGG |
| NNRRT_21nt_3- | AAGGAAGGAT | | AT |
| 12_015_- | | | |
| 31949397_saCas9 | | | |
| GuideDesign3_CBE_N | GGCTTTCATCTGGCAGA | 2187 | GAGA |
| NNRRT_21nt_3- | GAAGGAGAAT | | AT |
| 12_015_- | | | |
| 31950031_saCas9 | | | |
| GuideDesign3_CBE_N | CCTTACCTACGCTGACC | 2188 | TTGA |
| NNRRT_21nt_3- | TTGATTGAGT | | GT |
| 12_015_- | | | |
| 31950382_saCas9 | | | |
| GuideDesign3_CBE_N | TGCATCCTTACCTACGC | 2189 | CTTG |
| NNRRT_21nt_3- | TGACCTTGAT | | AT |
| 12_015_- | | | |
| 31950387_saCas9 | | | |
| GuideDesign3_CBE_N | TGTCTTACTTTGTTGCT | 2190 | AGTG |
| NNRRT_21nt_3- | GGCAAGTGGT | | GT |
| 12_015_- | | | |
| 31950925_saCas9 | | | |
| GuideDesign3_CBE_N | GTATGTCTTACTTTGTT | 2191 | GCAA |
| NNRRT_21nt_3- | GCTGGCAAGT | | GT |
| 12_015_- | | | |
| 31950928_saCas9 | | | |
| GuideDesign3_CBE_N | GTTTCTCACCTTATCCC | 2192 | CTTG |
| NNRRT_21nt_3- | CATTCTTGAT | | AT |
| 12_015_- | | | |
| 31951226_saCas9 | | | |
| GuideDesign3_CBE_N | TTCTCTCACCTCTGCAA | 2193 | TGGG |
| NNRRT_21nt_3- | GTATTGGGGT | | GT |
| 12_015_- | | | |
| 31951455_saCas9 | | | |
| GuideDesign3_CBE_N | TACACCAACCTGCAGAG | 2194 | TGGG |
| NNRRT_21nt_3- | GCAGTGGGGT | | GT |
| 12_015_- | | | |
| 31951856_saCas9 | | | |
| GuideDesign10_CBE_N | CACCTGAAGAGAAAGGC | 2195 | TGA |
| GA_20nt_4-9_006_- | TGATGA | | |
| 31946352_spCas9 | | | |
| GuideDesign10_CBE_N | TGGATTGCTTTGAGAAG | 2196 | GGA |
| GA_20nt_4-9_006_- | GGGGGA | | |
| 31946990_spCas9 | | | |
| GuideDesign10_CBE_N | TACCCCGCTGCAGAGGT | 2197 | AGA |
| GA_20nt_4-9_006_- | ATGAGA | | |
| 31947331_spCas9 | | | |
| GuideDesign10_CBE_N | GAGTCTGGGAGAGTCAG | 2198 | TGA |
| GA_20nt_4-9_006_- | AAATGA | | |
| 31947722_spCas9 | | | |
| GuideDesign10_CBE_N | TGGGCCTATAAAGGACA | 2199 | AGA |
| GA_20nt_4-9_006_- | AGGAGA | | |
| 31950849_spCas9 | | | |
| GuideDesign10_CBE_N | AATGGGCCTATAAAGGA | 2200 | GGA |
| GA_20nt_4-9_006_- | CAAGGA | | |
| 31950851_spCas9 | | | |
| GuideDesign10_CBE_N | GCCTTTCTGTAGGGGAG | 2201 | GGA |
| GA_20nt_4-9_006_- | GTGGGA | | |
| 31951323_spCas9 | | | |
| GuideDesign10_CBE_N | GTGGATTGCTTTGAGAA | 2202 | GGG |
| GG_20nt_4-9_003_- | GGGGGG | | |
| 31946991_spCas9 | | | |
| GuideDesign10_CBE_N | TGCAATCCTGCAGAAGA | 2203 | AGG |
| GG_20nt_4-9_003_- | GACAGG | | |
| 31949226_spCas9 | | | |
| GuideDesign10_CBE_N | CATCTGAGGGATAAAAA | 2204 | AGG |
| GG_20nt_4-9_003_- | GGAAGG | | |
| 31949399_spCas9 | | | |
| GuideDesign10_CBE_N | TAGACATCTGAGGGATA | 2205 | AGG |
| GG_20nt_4-9_003_- | AAAAGG | | |
| 31949403_spCas9 | | | |
| GuideDesign10_CBE_N | CCCTCCTGAAGTAGGAG | 2206 | AGG |
| GG_20nt_4-9_003_- | AGTAGG | | |
| 31950600_spCas9 | | | |
| GuideDesign10_CBE_N | AAATGGGCCTATAAAGG | 2207 | AGG |
| GG_20nt_4-9_003_- | ACAAGG | | |
| 31950852_spCas9 | | | |
| GuideDesign10_CBE_N | TGCCTTTCTGTAGGGGA | 2208 | GGG |
| GG_20nt_4-9_003_- | GGTGGG | | |
| 31951324_spCas9 | | | |
| GuideDesign10_CBE_N | ACCAACCTGCAGAGGCA | 2209 | GGG |
| GG_20nt_4-9_003_- | GTGGGG | | |
| 31951857_spCas9 | | | |
| GuideDesign3_ABE_N | CTTCTCACCTCCGTTGT | 2210 | AGA |
| GA_20nt_3-9_005_- | CACAGA | | |
| 31947177_spCas9 | | | |
| GuideDesign3_ABE_N | CTTCTCACCTCCGTTGT | 2211 | AGA |
| GA_20nt_3-9_005_- | CACAGA | | |
| 31947177_spCas9 | | | |
| GuideDesign3_ABE_N | CTGACCATCAGTCATGA | 2212 | TGA |
| GA_20nt_3-9_005_- | GGATGA | | |
| 31948943_spCas9 | | | |
| GuideDesign3_ABE_N | CTGACCATCAGTCATGA | 2213 | TGA |
| GA_20nt_3-9_005_- | GGATGA | | |
| 31948943_spCas9 | | | |
| GuideDesign3_ABE_N | CTCCCTACCGATCATTT | 2214 | AGA |
| GA_20nt_3-9_005_- | GGTAGA | | |
| 31949542_spCas9 | | | |
| GuideDesign3_ABE_N | CTCCCTACCGATCATTT | 2215 | AGA |
| GA_20nt_3-9_005_- | GGTAGA | | |
| 31949542_spCas9 | | | |
| GuideDesign3_ABE_N | TTACCTACGCTGACCTT | 2216 | TGA |
| GA_20nt_3-9_005_- | GATTGA | | |
| 31950384_spCas9 | | | |
| GuideDesign3_ABE_N | TTACCTACGCTGACCTT | 2217 | TGA |
| GA_20nt_3-9_005_- | GATTGA | | |
| 31950384_spCas9 | | | |
| GuideDesign3_ABE_N | ATCCTTACCTACGCTGA | 2218 | TGA |
| GA_20nt_3-9_005_- | CCTTGA | | |
| 31950388_spCas9 | | | |
| GuideDesign3_ABE_N | ATCCTTACCTACGCTGA | 2219 | TGA |
| GA_20nt_3-9_005_- | CCTTGA | | |
| 31950388_spCas9 | | | |
| GuideDesign3_ABE_N | TCTCACCTTATCCCCAT | 2220 | TGA |
| GA_20nt_3-9_005_- | TCTTGA | | |
| 31951227_spCas9 | | | |
| GuideDesign3_ABE_N | TCTCACCTTATCCCCAT | 2221 | TGA |
| GA_20nt_3-9_005_- | TCTTGA | | |
| 31951227_spCas9 | | | |
| GuideDesign3_ABE_N | GAGGACTCACTTGAATG | 2222 | CGA |
| GA_20nt_3-9_005_- | AAACGA | | |
| 31951591_spCas9 | | | |
| GuideDesign3_ABE_N | GAGGACTCACTTGAATG | 2223 | CGA |
| GA_20nt_3-9_005_- | AAACGA | | |
| 31951591_spCas9 | | | |
| GuideDesign3_ABE_N | TCAAAGCAATCCACTGT | 2224 | AGA |
| GA_20nt_3- | CCAAGA | | |
| 9_005_+_31947000_spC | | | |
| as9 | | | |
| GuideDesign3_ABE_N | TCAAAGCAATCCACTGT | 2225 | AGA |
| GA_20nt_3- | CCAAGA | | |
| 9_005_+_31947000_spC | | | |
| as9 | | | |
| GuideDesign3_ABE_N | CTCCCAGACTCCTTCAT | 2226 | CGA |
| GA_20nt_3- | GTACGA | | |
| 9_005_+_31947734_spC | | | |
| as9 | | | |
| GuideDesign3_ABE_N | CTCCCAGACTCCTTCAT | 2227 | CGA |
| GA_20nt_3- | GTACGA | | |
| 9_005_+_31947734_spC | | | |
| as9 | | | |
| GuideDesign3_ABE_N | CCCAGGGGAACAACAGA | 2228 | GGA |
| GA_20nt_3- | AGCGGA | | |
| 9_005_+_31947939_spC | | | |
| as9 | | | |
| GuideDesign3_ABE_N | CCCAGGGGAACAACAGA | 2229 | GGA |
| GA_20nt_3- | AGCGGA | | |
| 9_005_+_31947939_spC | | | |
| as9 | | | |
| GuideDesign3_ABE_N | ACAGGTGGCAAGTTATG | 2230 | TGA |
| GA_20nt_3- | GTGTGA | | |
| 9_005_+_31948369_spC | | | |
| as9 | | | |
| GuideDesign3_ABE_N | ACAGGTGGCAAGTTATG | 2231 | TGA |
| GA_20nt_3- | GTGTGA | | |
| 9_005_+_31948369_spC | | | |
| as9 | | | |
| GuideDesign3_ABE_N | GCCAGATGAAAGCCAGT | 2232 | TGA |
| GA_20nt_3- | CTCTGA | | |
| 9_005_+_31950044_spC | | | |
| as9 | | | |
| GuideDesign3_ABE_N | GCCAGATGAAAGCCAGT | 2233 | TGA |
| GA_20nt_3- | CTCTGA | | |
| 9_005_+_31950044_spC | | | |
| as9 | | | |
| GuideDesign3_ABE_N | GTCCCCAGCGCCCTTCA | 2234 | GGA |
| GA_20nt_3- | AAGGGA | | |
| 9_005_+_31950277_spC | | | |
| as9 | | | |
| GuideDesign3_ABE_N | GTCCCCAGCGCCCTTCA | 2235 | GGA |
| GA_20nt_3- | AAGGGA | | |
| 9_005_+_31950277_spC | | | |
| as9 | | | |
| GuideDesign3_ABE_N | TACAGAAAGGCAGCTGT | 2236 | AGA |
| GA_20nt_3- | GAGAGA | | |
| 9_005_+_31951335_spC | | | |
| as9 | | | |
| GuideDesign3_ABE_N | TACAGAAAGGCAGCTGT | 2237 | AGA |
| GA_20nt_3- | GAGAGA | | |
| 9_005_+_31951335_spC | | | |
| as9 | | | |
| GuideDesign3_ABE_N | TCGCTTACCTCCAGACA | 2238 | GGC |
| GC_20nt_3-9_008_- | AGAGGC | | |
| 31946270_spCas9 | | | |
| GuideDesign3_ABE_N | TCGCTTACCTCCAGACA | 2239 | GGC |
| GC_20nt_3-9_008_- | AGAGGC | | |
| 31946270_spCas9 | | | |
| GuideDesign3_ABE_N | TCACCTCCGTTGTCACA | 2240 | CGC |
| GC_20nt_3-9_008_- | GATCGC | | |
| 31947173_spCas9 | | | |
| GuideDesign3_ABE_N | TCACCTCCGTTGTCACA | 2241 | CGC |
| GC_20nt_3-9_008_- | GATCGC | | |
| 31947173_spCas9 | | | |
| GuideDesign3_ABE_N | CTTACAATGACTGAGAT | 2242 | GGC |
| GC_20nt_3-9_008_- | CTTGGC | | |
| 31950129_spCas9 | | | |
| GuideDesign3_ABE_N | CTTACAATGACTGAGAT | 2243 | GGC |
| GC_20nt_3-9_008_- | CTTGGC | | |
| 31950129_spCas9 | | | |
| GuideDesign3_ABE_N | ACGCTCTCACCTGATAG | 2244 | GGC |
| GC_20nt_3-9_008_- | TCTGGC | | |
| 31950759_spCas9 | | | |
| GuideDesign3_ABE_N | ACGCTCTCACCTGATAG | 2245 | GGC |
| GC_20nt_3-9_008_- | TCTGGC | | |
| 31950759_spCas9 | | | |
| GuideDesign3_ABE_N | CTGCAGGATTGCACAAC | 2246 | GGC |
| GC_20nt_3- | ATGGGC | | |
| 9_008_+_31949236_spC | | | |
| as9 | | | |
| GuideDesign3_ABE_N | CTGCAGGATTGCACAAC | 2247 | GGC |
| GC_20nt_3- | ATGGGC | | |
| 9_008_+_31949236_spC | | | |
| as9 | | | |
| GuideDesign3_ABE_N | ATAGGCCCATTTGTCTC | 2248 | TGC |
| GC_20nt_3- | CCCTGC | | |
| 9_008_+_31950863_spC | | | |
| as9 | | | |
| GuideDesign3_ABE_N | ATAGGCCCATTTGTCTC | 2249 | TGC |
| GC_20nt_3- | CCCTGC | | |
| 9_008_+_31950863_spC | | | |
| as9 | | | |
| GuideDesign3_ABE_N | CTCTGCAGGTTGGTGTA | 2250 | AGC |
| GC_20nt_3- | ATCAGC | | |
| 9_008_+_31951866_spC | | | |
| as9 | | | |
| GuideDesign3_ABE_N | CTCTGCAGGTTGGTGTA | 2251 | AGC |
| GC_20nt_3- | ATCAGC | | |
| 9_008_+_31951866_spC | | | |
| as9 | | | |
| GuideDesign3_ABE_N | CTCGCTTACCTCCAGAC | 2252 | AGG |
| GG_20nt_3-9_002_- | AAGAGG | | |
| 31946271_spCas9 | | | |
| GuideDesign3_ABE_N | CTCGCTTACCTCCAGAC | 2253 | AGG |
| GG_20nt_3-9_002_- | AAGAGG | | |
| 31946271_spCas9 | | | |
| GuideDesign3_ABE_N | CAAAGGTCACCTTGGCA | 2254 | AGG |
| GG_20nt_3-9_002_- | GGAAGG | | |
| 31947508_spCas9 | | | |
| GuideDesign3_ABE_N | CAAAGGTCACCTTGGCA | 2255 | AGG |
| GG_20nt_3-9_002_- | GGAAGG | | |
| 31947508_spCas9 | | | |
| GuideDesign3_ABE_N | CCTTCTGACCATCAGTC | 2256 | AGG |
| GG_20nt_3-9_002_- | ATGAGG | | |
| 31948947_spCas9 | | | |
| GuideDesign3_ABE_N | CCTTCTGACCATCAGTC | 2257 | AGG |
| GG_20nt_3-9_002_- | ATGAGG | | |
| 31948947_spCas9 | | | |
| GuideDesign3_ABE_N | TCACCCAGATAATCCTC | 2258 | TGG |
| GG_20nt_3-9_002_- | CCTTGG | | |
| 31949325_spCas9 | | | |
| GuideDesign3_ABE_N | TCACCCAGATAATCCTC | 2259 | TGG |
| GG_20nt_3-9_002_- | CCTTGG | | |
| 31949325_spCas9 | | | |
| GuideDesign3_ABE_N | GCTTACAATGACTGAGA | 2260 | TGG |
| GG_20nt_3-9_002_- | TCTTGG | | |
| 31950130_spCas9 | | | |
| GuideDesign3_ABE_N | GCTTACAATGACTGAGA | 2261 | TGG |
| GG_20nt_3-9_002_- | TCTTGG | | |
| 31950130_spCas9 | | | |
| GuideDesign3_ABE_N | CTTACTTTGTTGCTGGC | 2262 | TGG |
| GG_20nt_3-9_002_- | AAGTGG | | |
| 31950926_spCas9 | | | |
| GuideDesign3_ABE_N | CTTACTTTGTTGCTGGC | 2263 | TGG |
| GG_20nt_3-9_002_- | AAGTGG | | |
| 31950926_spCas9 | | | |
| GuideDesign3_ABE_N | TCTCACCTCTGCAAGTA | 2264 | GGG |
| GG_20nt_3-9_002_- | TTGGGG | | |
| 31951456_spCas9 | | | |
| GuideDesign3_ABE_N | TCTCACCTCTGCAAGTA | 2265 | GGG |
| GG_20nt_3-9_002_- | TTGGGG | | |
| 31951456_spCas9 | | | |
| GuideDesign3_ABE_N | CACCCACAGGTGGCAAG | 2266 | TGG |
| GG_20nt_3- | TTATGG | | |
| 9_002_+_31948364_spC | | | |
| as9 | | | |
| GuideDesign3_ABE_N | CACCCACAGGTGGCAAG | 2267 | TGG |
| GG_20nt_3- | TTATGG | | |
| 9_002_+_31948364_spC | | | |
| as9 | | | |
| GuideDesign3_ABE_N | ACAGACCACAAGTTGAA | 2268 | AGG |
| GG_20nt_3- | GTCAGG | | |
| 9_002_+_31948825_spC | | | |
| as9 | | | |
| GuideDesign3_ABE_N | ACAGACCACAAGTTGAA | 2269 | AGG |
| GG_20nt_3- | GTCAGG | | |
| 9_002_+_31948825_spC | | | |
| as9 | | | |
| GuideDesign3_ABE_N | TCTGCAGGATTGCACAA | 2270 | GGG |
| GG_20nt_3- | CATGGG | | |
| 9_002_+_31949235_spC | | | |
| as9 | | | |
| GuideDesign3_ABE_N | TCTGCAGGATTGCACAA | 2271 | GGG |
| GG_20nt_3- | CATGGG | | |
| 9_002_+_31949235_spC | | | |
| as9 | | | |
| GuideDesign3_ABE_N | GCAGGATTGCACAACAT | 2272 | CGG |
| GG_20nt_3- | GGGCGG | | |
| 9_002_+_31949238_spC | | | |
| as9 | | | |
| GuideDesign3_ABE_N | GCAGGATTGCACAACAT | 2273 | CGG |
| GG_20nt_3- | GGGCGG | | |
| 9_002_+_31949238_spC | | | |
| as9 | | | |
| GuideDesign3_ABE_N | CCCTCAGATGTCTATGT | 2274 | TGG |
| GG_20nt_3- | GTTTGG | | |
| 9_002_+_31949412_spC | | | |
| as9 | | | |
| GuideDesign3_ABE_N | CCCTCAGATGTCTATGT | 2275 | TGG |
| GG_20nt_3- | GTTTGG | | |
| 9_002_+_31949412_spC | | | |
| as9 | | | |
| GuideDesign3_ABE_N | CCTCAGATGTCTATGTG | 2276 | GGG |
| GG_20nt_3- | TTTGGG | | |
| 9_002_+_31949413_spC | | | |
| as9 | | | |
| GuideDesign3_ABE_N | CCTCAGATGTCTATGTG | 2277 | GGG |
| GG_20nt_3- | TTTGGG | | |
| 9_002_+_31949413_spC | | | |
| as9 | | | |
| GuideDesign3_ABE_N | CTCAGATGTCTATGTGT | 2278 | GGG |
| GG_20nt_3- | TTGGGG | | |
| 9_002_+_31949414_spC | | | |
| as9 | | | |
| GuideDesign3_ABE_N | CTCAGATGTCTATGTGT | 2279 | GGG |
| GG_20nt_3- | TTGGGG | | |
| 9_002_+_31949414_spC | | | |
| as9 | | | |
| GuideDesign3_ABE_N | GTGTCCCCAGCGCCCTT | 2280 | AGG |
| GG_20nt_3- | CAAAGG | | |
| 9_002_+_31950275_spC | | | |
| as9 | | | |
| GuideDesign3_ABE_N | GTGTCCCCAGCGCCCTT | 2281 | AGG |
| GG_20nt_3- | CAAAGG | | |
| 9_002_+_31950275_spC | | | |
| as9 | | | |
| GuideDesign3_ABE_N | TGTCCCCAGCGCCCTTC | 2282 | GGG |
| GG_20nt_3- | AAAGGG | | |
| 9_002_+_31950276_spC | | | |
| as9 | | | |
| GuideDesign3_ABE_N | TGTCCCCAGCGCCCTTC | 2283 | GGG |
| GG_20nt_3- | AAAGGG | | |
| 9_002_+_31950276_spC | | | |
| as9 | | | |
| GuideDesign3_ABE_N | CTACTTCAGGAGGGGAG | 2284 | CGG |
| GG_20nt_3- | AAGCGG | | |
| 9_002_+_31950609_spC | | | |
| as9 | | | |
| GuideDesign3_ABE_N | CTACTTCAGGAGGGGAG | 2285 | CGG |
| GG_20nt_3- | AAGCGG | | |
| 9_002_+_31950609_spC | | | |
| as9 | | | |
| GuideDesign3_ABE_N | TACTTCAGGAGGGGAGA | 2286 | GGG |
| GG_20nt_3- | AGCGGG | | |
| 9_002_+_31950610_spC | | | |
| as9 | | | |
| GuideDesign3_ABE_N | TACTTCAGGAGGGGAGA | 2287 | GGG |
| GG_20nt_3- | AGCGGG | | |
| 9_002_+_31950610_spC | | | |
| as9 | | | |
| GuideDesign3_ABE_N | CTCCTTGCAGGTGATTC | 2288 | CGG |
| GG_20nt_3- | TGGCGG | | |
| 9_002_+_31951544_spC | | | |
| as9 | | | |
| GuideDesign3_ABE_N | CTCCTTGCAGGTGATTC | 2289 | CGG |
| GG_20nt_3- | TGGCGG | | |
| 9_002_+_31951544_spC | | | |
| as9 | | | |
| GuideDesign3_ABE_N | TGCAGGTTGGTGTAATC | 2290 | TGG |
| GG_20nt_3- | AGCTGG | | |
| 9_002_+_31951869_spC | | | |
| as9 | | | |
| GuideDesign3_ABE_N | TGCAGGTTGGTGTAATC | 2291 | TGG |
| GG_20nt_3- | AGCTGG | | |
| 9_002_+_31951869_spC | | | |
| as9 | | | |
| GuideDesign3_ABE_N | GCAGGTTGGTGTAATCA | 2292 | GGG |
| GG_20nt_3- | GCTGGG | | |
| 9_002_+_31951870_spC | | | |
| as9 | | | |
| GuideDesign3_ABE_N | GCAGGTTGGTGTAATCA | 2293 | GGG |
| GG_20nt_3- | GCTGGG | | |
| 9_002_+_31951870_spC | | | |
| as9 | | | |
| GuideDesign3_ABE_N | TCCCTTCTGACCATCAG | 2294 | GAGG |
| NGRRT_21nt_5- | TCATGAGGAT | | AT |
| 14_011_- | | | |
| 31948945_saCas9 | | | |
| GuideDesign3_ABE_N | TCCCTTCTGACCATCAG | 2295 | GAGG |
| NGRRT_21nt_5- | TCATGAGGAT | | AT |
| 14_011_- | | | |
| 31948945_saCas9 | | | |
| GuideDesign3_ABE_N | ACTCACCCAGATAATCC | 2296 | TTGG |
| NGRRT_21nt_5- | TCCCTTGGGT | | GT |
| 14_011_- | | | |
| 31949323_saCas9 | | | |
| GuideDesign3_ABE_N | ACTCACCCAGATAATCC | 2297 | TTGG |
| NGRRT_21nt_5- | TCCCTTGGGT | | GT |
| 14_011_- | | | |
| 31949323_saCas9 | | | |
| GuideDesign3_ABE_N | CCTTACCTACGCTGACC | 2298 | TTGA |
| NGRRT_21nt_5- | TTGATTGAGT | | GT |
| 14_011_- | | | |
| 31950382_saCas9 | | | |
| GuideDesign3_ABE_N | CCTTACCTACGCTGACC | 2299 | TTGA |
| NGRRT_21nt_5- | TTGATTGAGT | | GT |
| 14_011_- | | | |
| 31950382_saCas9 | | | |
| GuideDesign3_ABE_N | TTCTCTCACCTCTGCAA | 2300 | TGGG |
| NGRRT_21nt_5- | GTATTGGGGT | | GT |
| 14_011_- | | | |
| 31951455_saCas9 | | | |
| GuideDesign3_ABE_N | TTCTCTCACCTCTGCAA | 2301 | TGGG |
| NGRRT_21nt_5- | GTATTGGGGT | | GT |
| 14_011_- | | | |
| 31951455_saCas9 | | | |
| GuideDesign3_ABE_N | TCCCTCAGATGTCTATG | 2302 | TGGG |
| NGRRT_21nt_5- | TGTTTGGGGT | | GT |
| 14_011_+_31949411_sa | | | |
| Cas9 | | | |
| GuideDesign3_ABE_N | TCCCTCAGATGTCTATG | 2303 | TGGG |
| NGRRT_21nt_5- | TGTTTGGGGT | | GT |
| 14_011_+_31949411_sa | | | |
| Cas9 | | | |
| GuideDesign3_ABE_N | CTGCCAGATGAAAGCCA | 2304 | CTGA |
| NGRRT_21nt_5- | GTCTCTGAGT | | GT |
| 14_011_+_31950042_sa | | | |
| Cas9 | | | |
| GuideDesign3_ABE_N | CTGCCAGATGAAAGCCA | 2305 | CTGA |
| NGRRT_21nt_5- | GTCTCTGAGT | | GT |
| 14_011_+_31950042_sa | | | |
| Cas9 | | | |
| GuideDesign3_ABE_N | ATGCTTCTCACCTCCGT | 2306 | ACAG |
| NNRRT_21nt_5- | TGTCACAGAT | | AT |
| 14_014_- | | | |
| 31947176_saCas9 | | | |
| GuideDesign3_ABE_N | ATGCTTCTCACCTCCGT | 2307 | ACAG |
| NNRRT_21nt_5- | TGTCACAGAT | | AT |
| 14_014_- | | | |
| 31947176_saCas9 | | | |
| GuideDesign3_ABE_N | AGGAGGATTCCACCTTC | 2308 | TTAA |
| NNRRT_21nt_5- | TCAATTAAGT | | GT |
| 14_014_- | | | |
| 31948067_saCas9 | | | |
| GuideDesign3_ABE_N | AGGAGGATTCCACCTTC | 2309 | TTAA |
| NNRRT_21nt_5- | TCAATTAAGT | | GT |
| 14_014_- | | | |
| 31948067_saCas9 | | | |
| GuideDesign3_ABE_N | CCCTAACCTCTGACCTT | 2310 | ATTG |
| NNRRT_21nt_5- | CATAATTGAT | | AT |
| 14_014_- | | | |
| 31948499_saCas9 | | | |
| GuideDesign3_ABE_N | CCCTAACCTCTGACCTT | 2311 | ATTG |
| NNRRT_21nt_5- | CATAATTGAT | | AT |
| 14_014_- | | | |
| 31948499_saCas9 | | | |
| GuideDesign3_ABE_N | CTTCTGACCATCAGTCA | 2312 | GATG |
| NNRRT_21nt_5- | TGAGGATGAT | | AT |
| 14_014_- | | | |
| 31948942_saCas9 | | | |
| GuideDesign3_ABE_N | CTTCTGACCATCAGTCA | 2313 | GATG |
| NNRRT_21nt_5- | TGAGGATGAT | | AT |
| 14_014_- | | | |
| 31948942_saCas9 | | | |
| GuideDesign3_ABE_N | CTTGTATCTCCCTACCG | 2314 | TTTG |
| NNRRT_21nt_5- | ATCATTTGGT | | GT |
| 14_014_- | | | |
| 31949545_saCas9 | | | |
| GuideDesign3_ABE_N | CTTGTATCTCCCTACCG | 2315 | TTTG |
| NNRRT_21nt_5- | ATCATTTGGT | | GT |
| 14_014_- | | | |
| 31949545_saCas9 | | | |
| GuideDesign3_ABE_N | GGATTCTGTGCTTACAA | 2316 | TGAG |
| NNRRT_21nt_5- | TGACTGAGAT | | AT |
| 14_014_- | | | |
| 31950135_saCas9 | | | |
| GuideDesign3_ABE_N | GGATTCTGTGCTTACAA | 2317 | TGAG |
| NNRRT_21nt_5- | TGACTGAGAT | | AT |
| 14_014_- | | | |
| 31950135_saCas9 | | | |
| GuideDesign3_ABE_N | TGCATCCTTACCTACGC | 2318 | CTTG |
| NNRRT_21nt_5- | TGACCTTGAT | | AT |
| 14_014_- | | | |
| 31950387_saCas9 | | | |
| GuideDesign3_ABE_N | TGCATCCTTACCTACGC | 2319 | CTTG |
| NNRRT_21nt_5- | TGACCTTGAT | | AT |
| 14_014_- | | | |
| 31950387_saCas9 | | | |
| GuideDesign3_ABE_N | TGTCTTACTTTGTTGCT | 2320 | AGTG |
| NNRRT_21nt_5- | GGCAAGTGGT | | GT |
| 14_014_- | | | |
| 31950925_saCas9 | | | |
| GuideDesign3_ABE_N | TGTCTTACTTTGTTGCT | 2321 | AGTG |
| NNRRT_21nt_5- | GGCAAGTGGT | | GT |
| 14_014_- | | | |
| 31950925_saCas9 | | | |
| GuideDesign3_ABE_N | GTATGTCTTACTTTGTT | 2322 | GCAA |
| NNRRT_21nt_5- | GCTGGCAAGT | | GT |
| 14_014_- | | | |
| 31950928_saCas9 | | | |
| GuideDesign3_ABE_N | GTATGTCTTACTTTGTT | 2323 | GCAA |
| NNRRT_21nt_5- | GCTGGCAAGT | | GT |
| 14_014_- | | | |
| 31950928_saCas9 | | | |
| GuideDesign3_ABE_N | CTTCAGGTGTGACCACC | 2324 | CATG |
| NNRRT_21nt_5- | ACTCCATGGT | | GT |
| 14_014_+_31946366_sa | | | |
| Cas9 | | | |
| GuideDesign3_ABE_N | CTTCAGGTGTGACCACC | 2325 | CATG |
| NNRRT_21nt_5- | ACTCCATGGT | | GT |
| 14_014_+_31946366_sa | | | |
| Cas9 | | | |
| GuideDesign3_ABE_N | CCCTCTCCACAGACCAC | 2326 | TGAA |
| NNRRT_21nt_5- | AAGTTGAAGT | | GT |
| 14_014_+_31948817_sa | | | |
| Cas9 | | | |
| GuideDesign3_ABE_N | CCCTCTCCACAGACCAC | 2327 | TGAA |
| NNRRT_21nt_5- | AAGTTGAAGT | | GT |
| 14_014_+_31948817_sa | | | |
| Cas9 | | | |
| GuideDesign3_ABE_N | CTACAGAAAGGCAGCTG | 2328 | AGAG |
| NNRRT_21nt_5- | TGAGAGAGAT | | AT |
| 14_014_+_31951334_sa | | | |
| Cas9 | | | |
| GuideDesign3_ABE_N | CTACAGAAAGGCAGCTG | 2329 | AGAG |
| NNRRT_21nt_5- | TGAGAGAGAT | | AT |
| 14_014_+_31951334_sa | | | |
| Cas9 | | | |
| GuideDesign3_CBE_N | TGTTGTTCCCCTGGGTG | 2330 | GAGA |
| NGRRT_21nt_3- | CCAGGAGAGT | | GT |
| 12_012_- | | | |
| 31947927_saCas9 | | | |
| GuideDesign3_CBE_N | TGTTGTTCCCCTGGGTG | 2331 | GAGA |
| NGRRT_21nt_3- | CCAGGAGAGT | | GT |
| 12_012_- | | | |
| 31947927_saCas9 | | | |
| GuideDesign3_CBE_N | GACATCTGAGGGATAAA | 2332 | AAGG |
| NGRRT_21nt_3- | AAGGAAGGAT | | AT |
| 12_012_- | | | |
| 31949397_saCas9 | | | |
| GuideDesign3_CBE_N | GACATCTGAGGGATAAA | 2333 | AAGG |
| NGRRT_21nt_3- | AAGGAAGGAT | | AT |
| 12_012_- | | | |
| 31949397_saCas9 | | | |
| GuideDesign3_CBE_N | GGCTTTCATCTGGCAGA | 2334 | GAGA |
| NGRRT_21nt_3- | GAAGGAGAAT | | AT |
| 12_012_- | | | |
| 31950031_saCas9 | | | |
| GuideDesign3_CBE_N | GGCTTTCATCTGGCAGA | 2335 | GAGA |
| NGRRT_21nt_3- | GAAGGAGAAT | | AT |
| 12_012_- | | | |
| 31950031_saCas9 | | | |
| GuideDesign3_CBE_N | TACACCAACCTGCAGAG | 2336 | TGGG |
| NGRRT_21nt_3- | GCAGTGGGGT | | GT |
| 12_012_- | | | |
| 31951856_saCas9 | | | |
| GuideDesign3_CBE_N | TACACCAACCTGCAGAG | 2337 | TGGG |
| NGRRT_21nt_3- | GCAGTGGGGT | | GT |
| 12_012_- | | | |
| 31951856_saCas9 | | | |
| GuideDesign3_CBE_N | CTTACCTCCAGACAAGA | 2338 | CAAG |
| NNRRT_21nt_3- | GGCCCAAGAT | | AT |
| 12_015_- | | | |
| 31946263_saCas9 | | | |
| GuideDesign3_CBE_N | CTTACCTCCAGACAAGA | 2339 | CAAG |
| NNRRT_21nt_3- | GGCCCAAGAT | | AT |
| 12_015_- | | | |
| 31946263_saCas9 | | | |
| GuideDesign3_CBE_N | TGGTCACACCTGAAGAG | 2340 | GCTG |
| NNRRT_21nt_3- | AAAGGCTGAT | | AT |
| 12_015_- | | | |
| 31946354_saCas9 | | | |
| GuideDesign3_CBE_N | TGGTCACACCTGAAGAG | 2341 | GCTG |
| NNRRT_21nt_3- | AAAGGCTGAT | | AT |
| 12_015_- | | | |
| 31946354_saCas9 | | | |
| GuideDesign3_CBE_N | GGATTGCTTTGAGAAGG | 2342 | ACAA |
| NNRRT_21nt_3- | GGGGACAAGT | | GT |
| 12_015_- | | | |
| 31946985_saCas9 | | | |
| GuideDesign3_CBE_N | GGATTGCTTTGAGAAGG | 2343 | ACAA |
| NNRRT_21nt_3- | GGGGACAAGT | | GT |
| 12_015_- | | | |
| 31946985_saCas9 | | | |
| GuideDesign3_CBE_N | CGCTGCAGAGGTATGAG | 2344 | CGAG |
| NNRRT_21nt_3- | ACATCGAGGT | | GT |
| 12_015_- | | | |
| 31947322_saCas9 | | | |
| GuideDesign3_CBE_N | CGCTGCAGAGGTATGAG | 2345 | CGAG |
| NNRRT_21nt_3- | ACATCGAGGT | | GT |
| 12_015_- | | | |
| 31947322_saCas9 | | | |
| GuideDesign3_CBE_N | GGAGTCTGGGAGAGTCA | 2346 | TGAG |
| NNRRT_21nt_3- | GAAATGAGGT | | GT |
| 12_015_- | | | |
| 31947719_saCas9 | | | |
| GuideDesign3_CBE_N | GGAGTCTGGGAGAGTCA | 2347 | TGAG |
| NNRRT_21nt_3- | GAAATGAGGT | | GT |
| 12_015_- | | | |
| 31947719_saCas9 | | | |
| GuideDesign3_CBE_N | ATGAAGGAGTCTGGGAG | 2348 | AGAA |
| NNRRT_21nt_3- | AGTCAGAAAT | | AT |
| 12_015_- | | | |
| 31947724_saCas9 | | | |
| GuideDesign3_CBE_N | ATGAAGGAGTCTGGGAG | 2349 | AGAA |
| NNRRT_21nt_3- | AGTCAGAAAT | | AT |
| 12_015_- | | | |
| 31947724_saCas9 | | | |
| GuideDesign3_CBE_N | TTTGAAGGGCGCTGGGG | 2350 | AACA |
| NNRRT_21nt_3- | ACACAACAGT | | GT |
| 12_015_- | | | |
| 31950269_saCas9 | | | |
| GuideDesign3_CBE_N | TTTGAAGGGCGCTGGGG | 2351 | AACA |
| NNRRT_21nt_3- | ACACAACAGT | | GT |
| 12_015_- | | | |
| 31950269_saCas9 | | | |
| GuideDesign3_CBE_N | CTCCCCTCCTGAAGTAG | 2352 | GTAG |
| NNRRT_21nt_3- | GAGAGTAGGT | | GT |
| 12_015_- | | | |
| 31950599_saCas9 | | | |
| GuideDesign3_CBE_N | CTCCCCTCCTGAAGTAG | 2353 | GTAG |
| NNRRT_21nt_3- | GAGAGTAGGT | | GT |
| 12_015_- | | | |
| 31950599_saCas9 | | | |
| GuideDesign3_CBE_N | CTTACTTTGTTGCTGGC | 2354 | GGTA |
| NNRRT_21nt_3- | AAGTGGTAGT | | GT |
| 12_015_- | | | |
| 31950922_saCas9 | | | |
| GuideDesign3_CBE_N | CTTACTTTGTTGCTGGC | 2355 | GGTA |
| NNRRT_21nt_3- | AAGTGGTAGT | | GT |
| 12_015_- | | | |
| 31950922_saCas9 | | | |
| GuideDesign10_CBE_N | ACTCCATCGGCCATTCA | 2356 | GGC |
| GC_20nt_4-9_009_- | CTTGGC | | |
| 31947141_spCas9 | | | |
| GuideDesign10_CBE_N | ACCCAAATTTTGGGGTA | 2357 | GGC |
| GC_20nt_4-9_009_- | TGTGGC | | |
| 31948418_spCas9 | | | |
| GuideDesign10_CBE_N | CGTGACCCAGTCTGCAT | 2358 | TGC |
| GC_20nt_4-9_009_- | TACTGC | | |
| 31948461_spCas9 | | | |
| GuideDesign10_CBE_N | GACCAAAAGACTGTCAG | 2359 | GGC |
| GC_20nt_4- | GAAGGC | | |
| 9_009_+_31946572_spC | | | |
| as9 | | | |
| GuideDesign10_CBE_N | GGCCGATGGAGTGGGCA | 2360 | AGC |
| GC_20nt_4- | GACAGC | | |
| 9_009_+_31947152_spC | | | |
| as9 | | | |
| GuideDesign10_CBE_N | ACCCCTCAAGAGGTGGC | 2361 | AGC |
| GC_20nt_4- | CGAAGC | | |
| 9_009_+_31947758_spC | | | |
| as9 | | | |
| GuideDesign10_CBE_N | AGGCCCTCCAGGCAGTG | 2362 | AGC |
| GC_20nt_4- | TACAGC | | |
| 9_009_+_31948862_spC | | | |
| as9 | | | |
| GuideDesign10_CBE_N | TGGCCAGACTATCAGGT | 2363 | AGC |
| GC_20nt_4- | GAGAGC | | |
| 9_009_+_31950757_spC | | | |
| as9 | | | |
| GuideDesign10_CBE_N | CAACAAAGTAAGACATA | 2364 | GGC |
| GC_20nt_4- | CTTGGC | | |
| 9_009_+_31950937_spC | | | |
| as9 | | | |
| GuideDesign10_CBE_N | CAAAAGCAGGTACCTGC | 2365 | CGC |
| GC_20nt_4- | TCACGC | | |
| 9_009_+_31951925_spC | | | |
| as9 | | | |
| GuideDesign10_CBE_N | CCTCTTTCAAGTGCTGC | 2366 | GGC |
| GC_20nt_4- | CCTGGC | | |
| 9_009_+_31951966_spC | | | |
| as9 | | | |
| GuideDesign3_CBE_N | GAGAAACTCCAAGATGA | 2367 | TTGG |
| NGRRT_21nt_3- | GGATTTGGGT | | GT |
| 12_012_+_31951994_sa | | | |
| Cas9 | | | |
| GuideDesign3_CBE_N | CTGCCATGGTTGCTTGT | 2368 | ATCG |
| NNRRT_21nt_3- | GGTAATCGGT | | GT |
| 12_015_- | | | |
| 31950102_saCas9 | | | |
| GuideDesign3_CBE_N | TTCTCCAAGAGGGCCAG | 2369 | TGGA |
| NNRRT_21nt_3- | GCACTGGAGT | | GT |
| 12_015_+_31946459_sa | | | |
| Cas9 | | | |
| GuideDesign3_CBE_N | ACCCTGTGCAGACACGT | 2370 | GCAG |
| NNRRT_21nt_3- | ACCTGCAGAT | | AT |
| 12_015_+_31946513_sa | | | |
| Cas9 | | | |
| GuideDesign3_CBE_N | ACCAAAAGACTGTCAGG | 2371 | CAGA |
| NNRRT_21nt_3- | AAGGCAGAGT | | GT |
| 12_015_+_31946573_sa | | | |
| Cas9 | | | |
| GuideDesign3_CBE_N | ACCTGCCAAGTGAATGG | 2372 | TGGA |
| NNRRT_21nt_3- | CCGATGGAGT | | GT |
| 12_015_+_31947137_sa | | | |
| Cas9 | | | |
| GuideDesign3_CBE_N | AGACAATGAGCAACATG | 2373 | CAAA |
| NNRRT_21nt_3- | TGTTCAAAGT | | GT |
| 12_015_+_31949486_sa | | | |
| Cas9 | | | |
| GuideDesign3_CBE_N | GAGCAACATGTGTTCAA | 2374 | AAGG |
| NNRRT_21nt_3- | AGTCAAGGAT | | AT |
| 12_015_+_31949493_sa | | | |
| Cas9 | | | |
| GuideDesign3_CBE_N | CCACAAGCAACCATGGC | 2375 | CAAG |
| NNRRT_21nt_3- | AGGCCAAGAT | | AT |
| 12_015_+_31950110_sa | | | |
| Cas9 | | | |
| GuideDesign3_CBE_N | CAAAAACCAGAAGCGGC | 2376 | GCAG |
| NNRRT_21nt_3- | AAAAGCAGGT | | GT |
| 12_015_+_31951909_sa | | | |
| Cas9 | | | |
| GuideDesign3_CBE_N | GAGAAACTCCAAGATGA | 2377 | TTGG |
| NNRRT_21nt_3- | GGATTTGGGT | | GT |
| 12_015_+_31951994_sa | | | |
| Cas9 | | | |
| GuideDesign10_CBE_N | AGGGTGCTCCAGGACCC | 2378 | AGA |
| GA_20nt_4-9_006_- | CGTAGA | | |
| 31946539_spCas9 | | | |
| GuideDesign10_CBE_N | GGGCCAGTATTCCCCGT | 2379 | CGA |
| GA_20nt_4-9_006_- | TCTCGA | | |
| 31947033_spCas9 | | | |
| GuideDesign10_CBE_N | CGCTCCAAGAGCCACCT | 2380 | TGA |
| GA_20nt_4-9_006_- | TCCTGA | | |
| 31947477_spCas9 | | | |
| GuideDesign10_CBE_N | GCGGTTCCAGCCTTCAG | 2381 | GGA |
| GA_20nt_4-9_006_- | GAGGGA | | |
| 31948907_spCas9 | | | |
| GuideDesign10_CBE_N | GTTCCCAAACCATGCCA | 2382 | AGA |
| GA_20nt_4-9_006_- | CAGAGA | | |
| 31950068_spCas9 | | | |
| GuideDesign10_CBE_N | GTGTTCCCAAACCATGC | 2383 | AGA |
| GA_20nt_4-9_006_- | CACAGA | | |
| 31950070_spCas9 | | | |
| GuideDesign10_CBE_N | CAGCCAGGGCAGCACTT | 2384 | AGA |
| GA_20nt_4-9_006_- | GAAAGA | | |
| 31951968_spCas9 | | | |
| GuideDesign10_CBE_N | CCTTCAGCCAGGGCAGC | 2385 | TGA |
| GA_20nt_4-9_006_- | ACTTGA | | |
| 31951972_spCas9 | | | |
| GuideDesign10_CBE_N | CCCCAGGGATCCTGCTC | 2386 | GGA |
| GA_20nt_4- | TCTGGA | | |
| 9_006_+_31946404_spC | | | |
| as9 | | | |
| GuideDesign10_CBE_N | TCAAGACCAAAAGACTG | 2387 | GGA |
| GA_20nt_4- | TCAGGA | | |
| 9_006_+_31946568_spC | | | |
| as9 | | | |
| GuideDesign10_CBE_N | GCACCTGCCAAGTGAAT | 2388 | CGA |
| GA_20nt_4- | GGCCGA | | |
| 9_006_+_31947135_spC | | | |
| as9 | | | |
| GuideDesign10_CBE_N | CTGCCAAGTGAATGGCC | 2389 | GGA |
| GA_20nt_4- | GATGGA | | |
| 9_006_+_31947139_spC | | | |
| as9 | | | |
| GuideDesign10_CBE_N | GAATGGCCGATGGAGTG | 2390 | AGA |
| GA_20nt_4- | GGCAGA | | |
| 9_006_+_31947148_spC | | | |
| as9 | | | |
| GuideDesign10_CBE_N | AGTGGGCAGACAGCGAT | 2391 | TGA |
| GA_20nt_4- | CTGTGA | | |
| 9_006_+_31947161_spC | | | |
| as9 | | | |
| GuideDesign10_CBE_N | GGCAGCCAGTACCGCCT | 2392 | AGA |
| GA_20nt_4- | TGAAGA | | |
| 9_006_+_31947394_spC | | | |
| as9 | | | |
| GuideDesign10_CBE_N | TCCCAGCGGCGAACGTG | 2393 | GGA |
| GA_20nt_4- | TCAGGA | | |
| 9_006_+_31947460_spC | | | |
| as9 | | | |
| GuideDesign10_CBE_N | GTGTCAGGAAGGTGGCT | 2394 | GGA |
| GA_20nt_4- | CTTGGA | | |
| 9_006_+_31947474_spC | | | |
| as9 | | | |
| GuideDesign10_CBE_N | AGGGGAACAACAGAAGC | 2395 | AGA |
| GA_20nt_4- | GGAAGA | | |
| 9_006_+_31947942_spC | | | |
| as9 | | | |
| GuideDesign10_CBE_N | CCTCCAGGCAGTGTACA | 2396 | TGA |
| GA_20nt_4- | GCATGA | | |
| 9_006_+_31948866_spC | | | |
| as9 | | | |
| GuideDesign10_CBE_N | TTCTACCAAATGATCGG | 2397 | GGA |
| GA_20nt_4- | TAGGGA | | |
| 9_006_+_31949541_spC | | | |
| as9 | | | |
| GuideDesign10_CBE_N | CTACCAAATGATCGGTA | 2398 | AGA |
| GA_20nt_4- | GGGAGA | | |
| 9_006_+_31949543_spC | | | |
| as9 | | | |
| GuideDesign10_CBE_N | CAAGCAACCATGGCAGG | 2399 | AGA |
| GA_20nt_4- | CCAAGA | | |
| 9_006_+_31950113_spC | | | |
| as9 | | | |
| GuideDesign10_CBE_N | TATGGCCAGACTATCAG | 2400 | AGA |
| GA_20nt_4- | GTGAGA | | |
| 9_006_+_31950755_spC | | | |
| as9 | | | |
| GuideDesign10_CBE_N | CACTTGCCAGCAACAAA | 2401 | AGA |
| GA_20nt_4- | GTAAGA | | |
| 9_006_+_31950927_spC | | | |
| as9 | | | |
| GuideDesign10_CBE_N | GCTCAATATGCCCCAGG | 2402 | TGA |
| GA_20nt_4- | CTATGA | | |
| 9_006_+_31951361_spC | | | |
| as9 | | | |
| GuideDesign10_CBE_N | CTTTCAAGTGCTGCCCT | 2403 | TGA |
| GA_20nt_4- | GGCTGA | | |
| 9_006_+_31951969_spC | | | |
| as9 | | | |
| GuideDesign10_CBE_N | CACTCCATCGGCCATTC | 2404 | TGG |
| GG_20nt_4-9_003_- | ACTTGG | | |
| 31947142_spCas9 | | | |
| GuideDesign10_CBE_N | GACCCAAATTTTGGGGT | 2405 | TGG |
| GG_20nt_4-9_003_- | ATGTGG | | |
| 31948419_spCas9 | | | |
| GuideDesign10_CBE_N | TGCGGTTCCAGCCTTCA | 2406 | GGG |
| GG_20nt_4-9_003_- | GGAGGG | | |
| 31948908_spCas9 | | | |
| GuideDesign10_CBE_N | GTGCGGTTCCAGCCTTC | 2407 | AGG |
| GG_20nt_4-9_003_- | AGGAGG | | |
| 31948909_spCas9 | | | |
| GuideDesign10_CBE_N | TGGCCTGCCATGGTTGC | 2408 | TGG |
| GG_20nt_4-9_003_- | TTGTGG | | |
| 31950110_spCas9 | | | |
| GuideDesign10_CBE_N | CTCCTTCCGACTTCTCC | 2409 | AGG |
| GG_20nt_4- | AAGAGG | | |
| 9_003_+_31946448_spC | | | |
| as9 | | | |
| GuideDesign10_CBE_N | TCCTTCCGACTTCTCCA | 2410 | GGG |
| GG_20nt_4- | AGAGGG | | |
| 9_003_+_31946449_spC | | | |
| as9 | | | |
| GuideDesign10_CBE_N | TCTCCAAGAGGGCCAGG | 2411 | TGG |
| GG_20nt_4- | CACTGG | | |
| 9_003_+_31946460_spC | | | |
| as9 | | | |
| GuideDesign10_CBE_N | CTCAAGACCAAAAGACT | 2412 | AGG |
| GG_20nt_4- | GTCAGG | | |
| 9_003_+_31946567_spC | | | |
| as9 | | | |
| GuideDesign10_CBE_N | AGACCAAAAGACTGTCA | 2413 | AGG |
| GG_20nt_4- | GGAAGG | | |
| 9_003_+_31946571_spC | | | |
| as9 | | | |
| GuideDesign10_CBE_N | CCTGCCAAGTGAATGGC | 2414 | TGG |
| GG_20nt_4- | CGATGG | | |
| 9_003_+_31947138_spC | | | |
| as9 | | | |
| GuideDesign10_CBE_N | CTCCCAGCGGCGAACGT | 2415 | AGG |
| GG_20nt_4- | GTCAGG | | |
| 9_003_+_31947459_spC | | | |
| as9 | | | |
| GuideDesign10_CBE_N | CAGCGGCGAACGTGTCA | 2416 | AGG |
| GG_20nt_4- | GGAAGG | | |
| 9_003_+_31947463_spC | | | |
| as9 | | | |
| GuideDesign10_CBE_N | CGGCGAACGTGTCAGGA | 2417 | TGG |
| GG_20nt_4- | AGGTGG | | |
| 9_003_+_31947466_spC | | | |
| as9 | | | |
| GuideDesign10_CBE_N | CGTGTCAGGAAGGTGGC | 2418 | TGG |
| GG_20nt_4- | TCTTGG | | |
| 9_003_+_31947473_spC | | | |
| as9 | | | |
| GuideDesign10_CBE_N | TTTTCTACCAAATGATC | 2419 | AGG |
| GG_20nt_4- | GGTAGG | | |
| 9_003_+_31949539_spC | | | |
| as9 | | | |
| GuideDesign10_CBE_N | TTTCTACCAAATGATCG | 2420 | GGG |
| GG_20nt_4- | GTAGGG | | |
| 9_003_+_31949540_spC | | | |
| as9 | | | |
| GuideDesign10_CBE_N | GCAACAAAGTAAGACAT | 2421 | TGG |
| GG_20nt_4- | ACTTGG | | |
| 9_003_+_31950936_spC | | | |
| as9 | | | |
| GuideDesign10_CBE_N | ACCTCTTTCAAGTGCTG | 2422 | TGG |
| GG_20nt_4- | CCCTGG | | |
| 9_003_+_31951965_spC | | | |
| as9 | | | |
| GuideDesign10_CBE_N | GAAACTCCAAGATGAGG | 2423 | TGG |
| GG_20nt_4- | ATTTGG | | |
| 9_003_+_31951996_spC | | | |
| as9 | | | |
| GuideDesign10_CBE_N | AAACTCCAAGATGAGGA | 2424 | GGG |
| GG_20nt_4- | TTTGGG | | |
| 9_003_+_31951997_spC | | | |
| as9 | | | |
| GuideDesign3_CBE_N | TTCTCCAAGAGGGCCAG | 2425 | TGGA |
| NGRRT_21nt_3- | GCACTGGAGT | | GT |
| 12_012_+_31946459_sa | | | |
| Cas9 | | | |
| GuideDesign3_CBE_N | TTCTCCAAGAGGGCCAG | 2426 | TGGA |
| NGRRT_21nt_3- | GCACTGGAGT | | GT |
| 12_012_+_31946459_sa | | | |
| Cas9 | | | |
| GuideDesign3_CBE_N | ACCAAAAGACTGTCAGG | 2427 | CAGA |
| NGRRT_21nt_3- | AAGGCAGAGT | | GT |
| 12_012_+_31946573_sa | | | |
| Cas9 | | | |
| GuideDesign3_CBE_N | ACCAAAAGACTGTCAGG | 2428 | CAGA |
| NGRRT_21nt_3- | AAGGCAGAGT | | GT |
| 12_012_+_31946573_sa | | | |
| Cas9 | | | |
| GuideDesign3_CBE_N | ACCTGCCAAGTGAATGG | 2429 | TGGA |
| NGRRT_21nt_3- | CCGATGGAGT | | GT |
| 12_012_+_31947137_sa | | | |
| Cas9 | | | |
| GuideDesign3_CBE_N | ACCTGCCAAGTGAATGG | 2430 | TGGA |
| NGRRT_21nt_3- | CCGATGGAGT | | GT |
| 12_012_+_31947137_sa | | | |
| Cas9 | | | |
| GuideDesign3_CBE_N | GAGCAACATGTGTTCAA | 2431 | AAGG |
| NGRRT_21nt_3- | AGTCAAGGAT | | AT |
| 12_012_+_31949493_sa | | | |
| Cas9 | | | |
| GuideDesign3_CBE_N | GAGCAACATGTGTTCAA | 2432 | AAGG |
| NGRRT_21nt_3- | AGTCAAGGAT | | AT |
| 12_012_+_31949493_sa | | | |
| Cas9 | | | |
| GuideDesign3_CBE_N | GCTCCAGGACCCCGTAG | 2433 | GCAG |
| NNRRT_21nt_3- | ATCTGCAGGT | | GT |
| 12_015_- | | | |
| 31946530_saCas9 | | | |
| GuideDesign3_CBE_N | GCTCCAGGACCCCGTAG | 2434 | GCAG |
| NNRRT_21nt_3- | ATCTGCAGGT | | GT |
| 12_015_- | | | |
| 31946530_saCas9 | | | |
| GuideDesign3_CBE_N | TTCAGGGTGCTCCAGGA | 2435 | GTAG |
| NNRRT_21nt_3- | CCCCGTAGAT | | AT |
| 12_015_- | | | |
| 31946538_saCas9 | | | |
| GuideDesign3_CBE_N | TTCAGGGTGCTCCAGGA | 2436 | GTAG |
| NNRRT_21nt_3- | CCCCGTAGAT | | AT |
| 12_015_- | | | |
| 31946538_saCas9 | | | |
| GuideDesign3_CBE_N | GGGGCCAGTATTCCCCG | 2437 | CGAA |
| NNRRT_21nt_3- | TTCTCGAAGT | | GT |
| 12_015_- | | | |
| 31947030_saCas9 | | | |
| GuideDesign3_CBE_N | GGGGCCAGTATTCCCCG | 2438 | CGAA |
| NNRRT_21nt_3- | TTCTCGAAGT | | GT |
| 12_015_- | | | |
| 31947030_saCas9 | | | |
| GuideDesign3_CBE_N | ACTCCATCGGCCATTCA | 2439 | GCAG |
| NNRRT_21nt_3- | CTTGGCAGGT | | GT |
| 12_015_- | | | |
| 31947137_saCas9 | | | |
| GuideDesign3_CBE_N | ACTCCATCGGCCATTCA | 2440 | GCAG |
| NNRRT_21nt_3- | CTTGGCAGGT | | GT |
| 12_015_- | | | |
| 31947137_saCas9 | | | |
| GuideDesign3_CBE_N | TGGCCTGCCATGGTTGC | 2441 | GGTA |
| NNRRT_21nt_3- | TTGTGGTAAT | | AT |
| 12_015_- | | | |
| 31950106_saCas9 | | | |
| GuideDesign3_CBE_N | TGGCCTGCCATGGTTGC | 2442 | GGTA |
| NNRRT_21nt_3- | TTGTGGTAAT | | AT |
| 12_015_- | | | |
| 31950106_saCas9 | | | |
| GuideDesign3_CBE_N | TCTTGGCCTGCCATGGT | 2443 | TGTG |
| NNRRT_21nt_3- | TGCTTGTGGT | | GT |
| 12_015_- | | | |
| 31950109_saCas9 | | | |
| GuideDesign3_CBE_N | TCTTGGCCTGCCATGGT | 2444 | TGTG |
| NNRRT_21nt_3- | TGCTTGTGGT | | GT |
| 12_015_- | | | |
| 31950109_saCas9 | | | |
| GuideDesign3_CBE_N | TCAGCCAGGGCAGCACT | 2445 | AGAG |
| NNRRT_21nt_3- | TGAAAGAGGT | | GT |
| 12_015_- | | | |
| 31951965_saCas9 | | | |
| GuideDesign3_CBE_N | TCAGCCAGGGCAGCACT | 2446 | AGAG |
| NNRRT_21nt_3- | TGAAAGAGGT | | GT |
| 12_015_- | | | |
| 31951965_saCas9 | | | |
| GuideDesign3_CBE_N | ATCGCACCTGCCAAGTG | 2447 | GCCG |
| NNRRT_21nt_3- | AATGGCCGAT | | AT |
| 12_015_+_31947132_sa | | | |
| Cas9 | | | |
| GuideDesign3_CBE_N | ATCGCACCTGCCAAGTG | 2448 | GCCG |
| NNRRT_21nt_3- | AATGGCCGAT | | AT |
| 12_015_+_31947132_sa | | | |
| Cas9 | | | |
| GuideDesign3_CBE_N | TGGCCGATGGAGTGGGC | 2449 | AGCG |
| NNRRT_21nt_3- | AGACAGCGAT | | AT |
| 12_015_+_31947151_sa | | | |
| Cas9 | | | |
| GuideDesign3_CBE_N | TGGCCGATGGAGTGGGC | 2450 | AGCG |
| NNRRT_21nt_3- | AGACAGCGAT | | AT |
| 12_015_+_31947151_sa | | | |
| Cas9 | | | |
| GuideDesign3_CBE_N | TCCCAGCGGCGAACGTG | 2451 | GAAG |
| NNRRT_21nt_3- | TCAGGAAGGT | | GT |
| 12_015_+_31947460_sa | | | |
| Cas9 | | | |
| GuideDesign3_CBE_N | TCCCAGCGGCGAACGTG | 2452 | GAAG |
| NNRRT_21nt_3- | TCAGGAAGGT | | GT |
| 12_015_+_31947460_sa | | | |
| Cas9 | | | |
| GuideDesign3_CBE_N | CCCAGGGGAACAACAGA | 2453 | GAAG |
| NNRRT_21nt_3- | AGCGGAAGAT | | AT |
| 12_015_+_31947939_sa | | | |
| Cas9 | | | |
| GuideDesign3_CBE_N | CCCAGGGGAACAACAGA | 2454 | GAAG |
| NNRRT_21nt_3- | AGCGGAAGAT | | AT |
| 12_015_+_31947939_sa | | | |
| Cas9 | | | |
| GuideDesign3_CBE_N | GTCACGAAGCAGCTCAA | 2455 | ATCA |
| NNRRT_21nt_3- | TGAAATCAAT | | AT |
| 12_015_+_31948478_sa | | | |
| Cas9 | | | |
| GuideDesign3_CBE_N | GTCACGAAGCAGCTCAA | 2456 | ATCA |
| NNRRT_21nt_3- | TGAAATCAAT | | AT |
| 12_015_+_31948478_sa | | | |
| Cas9 | | | |
| GuideDesign3_CBE_N | GGCCCTCCAGGCAGTGT | 2457 | CATG |
| NNRRT_21nt_3- | ACAGCATGAT | | AT |
| 12_015_+_31948863_sa | | | |
| Cas9 | | | |
| GuideDesign3_CBE_N | GGCCCTCCAGGCAGTGT | 2458 | CATG |
| NNRRT_21nt_3- | ACAGCATGAT | | AT |
| 12_015_+_31948863_sa | | | |
| Cas9 | | | |
| GuideDesign3_CBE_N | TTTCTACCAAATGATCG | 2459 | GGAG |
| NNRRT_21nt_3- | GTAGGGAGAT | | AT |
| 12_015_+_31949540_sa | | | |
| Cas9 | | | |
| GuideDesign3_CBE_N | TTTCTACCAAATGATCG | 2460 | GGAG |
| NNRRT_21nt_3- | GTAGGGAGAT | | AT |
| 12_015_+_31949540_sa | | | |
| Cas9 | | | |
| GuideDesign3_CBE_N | GCAACCATGGCAGGCCA | 2461 | CTCA |
| NNRRT_21nt_3- | AGATCTCAGT | | GT |
| 12_015_+_31950116_sa | | | |
| Cas9 | | | |
| GuideDesign3_CBE_N | GCAACCATGGCAGGCCA | 2462 | CTCA |
| NNRRT_21nt_3- | AGATCTCAGT | | GT |
| 12_015_+_31950116_sa | | | |
| Cas9 | | | |

TABLE 2F
Representative spacer and target site sequences relating to altering a TATA
box or start codon of a complement factor B polynucleotide using base editing.

| Guide | Target | | | | SEQ | | | |
|---|---|---|---|---|---|---|---|---|
| poly- | site | | Cas | | ID | | SEQ | |
| nucleotide | des- | | des- | Target | NO | Spacer | ID | PAM |
| name | cription | Editor | cription | Site | | sequence | NO | sequence |
| GuideDesign90_ | TATA | ABE_NGA_ | spCas9 | AAAAGGC | 2871 | AAAAGGCU | 3177 | GGA |
| ABE_NGA_ | box | 20 nt_3- | VRQR | TGGGGGC | | GGGGGCUG | | |
| 20 nt 3- | | 12_019 | IBE | TGGTGGG | | GUGG | | |
| 12_019_+_ | | | | GA | | | | |
| 31946067_spCas9 | | | | | | | | |
| GuideDesign90_ | TATA | CBE_NGA_ | spCas9 | AAAAGGC | 2872 | AAAAGGCU | 3178 | GGA |
| CBE_NGA_ | box | 20 nt_4- | VRQR | TGGGGGC | | GGGGGCUG | | |
| 20 nt_4- | | 9_006 | CBE | TGGTGGG | | GUGG | | |
| 9_006_+_ | | | | GA | | | | |
| 31946067_spCas9 | | | | | | | | |
| GuideDesign90_ | TATA | CBE_NGA_ | spCas9 | AAAAGGC | 2873 | AAAAGGCU | 3179 | GGA |
| CBE_NGA_ | box | 20 nt_3- | IBE | TGGGGGC | | GGGGGCUG | | |
| 20 nt_3- | | 16_035 | extended | TGGTGGG | | GUGG | | |
| 16_035_+_ | | | CBE | GA | | | | |
| 31946067_spCas9 | | | | | | | | |
| GuideDesign90_ | TATA | ABE_NG_ | spCas9 | AAAAGGC | 2874 | AAAAGGCU | 3180 | GG |
| ABE_NG_ | box | 20 nt 3- | NG | TGGGGGC | | GGGGGCUG | | |
| 20 nt_3- | | 9_027 | ABE | TGGTGGG | | GUGG | | |
| 9_027_+_ | | | | G | | | | |
| 31946067_spCas9 | | | | | | | | |
| GuideDesign90_ | TATA | CBE_NG_ | spCas9 | AAAAGGC | 2875 | AAAAGGCU | 3181 | GG |
| CBE_NG_ | box | 20 nt_4- | NG | TGGGGGC | | GGGGGCUG | | |
| 20 nt_4- | | 9_028 | CBE | TGGTGGG | | GUGG | | |
| 9_028_+_ | | | | G | | | | |
| 31946067_spCas9 | | | | | | | | |
| GuideDesign | TATA | ABE_NRN_ | spCas9 | AAAAGGC | 2876 | AAAAGGCU | 3182 | GGA |
| 90_ABE_NRN_ | box | 20 nt_3- | NRN | TGGGGGC | | GGGGGCUG | | |
| 20 nt_3- | | 9_021 | ABE | TGGTGGG | | GUGG | | |
| 9_021_+_ | | | | GA | | | | |
| 31946067_spCas9 | | | | | | | | |
| GuideDesign90_ | TATA | CBE_NRN_ | spCas9 | AAAAGGC | 2877 | AAAAGGCU | 3183 | GGA |
| CBE_NRN_ | box | 20 nt_3- | NRN | TGGGGGC | | GGGGGCUG | | |
| 20 nt_3- | | 9_022 | CBE | TGGTGGG | | GUGG | | |
| 9_022_+_ | | | | GA | | | | |
| 31946067_spCas9 | | | | | | | | |
| GuideDesign90_ | TATA | MBE_NRN_ | spCas9 | AAAAGGC | 2878 | AAAAGGCU | 3184 | GGA |
| MBE_NRN_ | box | 20 nt_3- | NRN | TGGGGGC | | GGGGGCUG | | |
| 20 nt_3- | | 9_023 | MBE | TGGTGGG | | GUGG | | |
| 9_023_+_ | | | | GA | | | | |
| 31946067_spCas9 | | | | | | | | |
| GuideDesign90_ | TATA | ABE_NRN_ | spCas9 | AAAGGCT | 2879 | AAAGGCUG | 3185 | GAG |
| ABE_NRN_ | box | 20 nt_3- | NRN | GGGGGCT | | GGGGCUGG | | |
| 20 nt_3- | | 9_021 | ABE | GGTGGGG | | UGGG | | |
| 9_021_+_ | | | | AG | | | | |
| 31946068_spCas9 | | | | | | | | |
| GuideDesign90_ | TATA | CBE_NRN_ | spCas9 | AAAGGCT | 2880 | AAAGGCUG | 3186 | GAG |
| CBE_NRN_ | box | 20 nt_3- | NRN | GGGGGCT | | GGGGCUGG | | |
| 20 nt_3- | | 9_022 | CBE | GGTGGGG | | UGGG | | |
| 9_022_+_ | | | | AG | | | | |
| 31946068_spCas9 | | | | | | | | |
| GuideDesign90_ | TATA | MBE_NRN_ | spCas9 | AAAGGCT | 2881 | AAAGGCUG | 3187 | GAG |
| MBE_NRN_ | box | 20 nt 3- | NRN | GGGGGCT | | GGGGCUGG | | |
| 20 nt_3- | | 9_023 | MBE | GGTGGGG | | UGGG | | |
| 9_023_+_ | | | | AG | | | | |
| 31946068_spCas9 | | | | | | | | |
| GuideDesign90_ | TATA | CBE_NRRH_ | spCas9 | AAAGGCT | 2882 | AAAGGCUG | 3188 | GAGC |
| CBE_NRRH_ | box | 20 nt 3- | NRRH | GGGGGCT | | GGGGCUGG | | |
| 20 nt_3- | | 9_031 | CBE | GGTGGGG | | UGGG | | |
| 9_031_+_ | | | | AGC | | | | |
| 31946068_spCas9 | | | | | | | | |
| GuideDesign9 | TATA | ABE_NGC_ | spCas9 | AAGGCTG | 2883 | AAGGCUGG | 3189 | AGC |
| ABE_NGC0_ | box | 20 nt 3- | NGC | GGGGCTG | | GGGCUGGU | | |
| 20 nt_3- | | 9_008 | ABE | GTGGGGA | | GGGG | | |
| 9_008_+_ | | | | GC | | | | |
| 31946069_spCas9 | | | | | | | | |
| GuideDesign90_ | TATA | ABE_NGC_ | spCas9 | AAGGCTG | 2884 | AAGGCUGG | 3190 | AGC |
| ABE_NGC_ | box | 20 nt_3- | NGC | GGGGCTG | | GGGCUGGU | | |
| 20 nt_3- | | 12_020 | IBE | GTGGGGA | | GGGG | | |
| 12_020_+_ | | | | GC | | | | |
| 31946069_spCas9 | | | | | | | | |
| GuideDesign90_ | TATA | CBE_NGC_ | spCas9 | AAGGCTG | 2885 | AAGGCUGG | 3191 | AGC |
| CBE_NGC_ | box | 20 nt_4- | NGC | GGGGCTG | | GGGCUGGU | | |
| 20 nt_4- | | 9_009 | CBE | GTGGGGA | | GGGG | | |
| 9_009_+_ | | | | GC | | | | |
| 31946069_spCas9 | | | | | | | | |
| GuideDesign90_ | TATA | ABE NG_ | spCas9 | AAGGCTG | 2886 | AAGGCUGG | 3192 | AG |
| ABE_NG_ | box | 20 nt_3- | NG | GGGGCTG | | GGGCUGGU | | |
| 20 nt_3- | | 9_027 | ABE | GTGGGGA | | GGGG | | |
| 9_027_+_ | | | | G | | | | |
| 31946069_spCas9 | | | | | | | | |
| GuideDesign90_ | TATA | CBE_NG_ | spCas9 | AAGGCTG | 2887 | AAGGCUGG | 3193 | AG |
| CBE_NG_ | box | 20 nt_4- | NG | GGGGCTG | | GGGCUGGU | | |
| 20 nt_4- | | 9_028 | CBE | GTGGGGA | | GGGG | | |
| 9_028_+_ | | | | G | | | | |
| 31946069_spCas9 | | | | | | | | |
| GuideDesign90_ | TATA | ABE_NRN_ | spCas9 | AAGGCTG | 2888 | AAGGCUGG | 3194 | AGC |
| ABE_NRN_ | box | 20 nt_3- | NRN | GGGGCTG | | GGGCUGGU | | |
| 20 nt_3- | | 9_021 | ABE | GTGGGGA | | GGGG | | |
| 9_021_+_ | | | | GC | | | | |
| 31946069_spCas9 | | | | | | | | |
| GuideDesign90_ | TATA | CBE_NRN_ | spCas9 | AAGGCTG | 2889 | AAGGCUGG | 3195 | AGC |
| CBE_NRN_ | box | 20 nt_3- | NRN | GGGGCTG | | GGGCUGGU | | |
| 20 nt_3- | | 9_022 | CBE | GTGGGGA | | GGGG | | |
| 9_022_+_ | | | | GC | | | | |
| 31946069_spCas9 | | | | | | | | |
| GuideDesign90_ | TATA | MBE_NRN_ | spCas9 | AAGGCTG | 2890 | AAGGCUGG | 3196 | AGC |
| MBE_NRN_ | box | 20 nt_3- | NRN | GGGGCTG | | GGGCUGGU | | |
| 20 nt_3- | | 9_023 | MBE | GTGGGGA | | GGGG | | |
| 9_023_+_ | | | | GC | | | | |
| 31946069_spCas9 | | | | | | | | |
| Guide_Design90_ | TATA | ABE_NRCH_ | spCas9 | AAGGCTG | 2891 | AAGGCUGG | 3197 | AGCA |
| ABE_NRCH_ | box | 20 nt_3- | NRCH | GGGGCTG | | GGGCUGGU | | |
| 20 nt_3- | | 9_024 | ABE | GTGGGGA | | GGGG | | |
| 9_024_+_ | | | | GCA | | | | |
| 31946069_spCas9 | | | | | | | | |
| GuideDesign90_ | TATA | CBE_NRCH_ | spCas9 | AAGGCTG | 2892 | AAGGCUGG | 3198 | AGCA |
| CBE_NRCH_ | box | 20 nt_3- | NRCH | GGGGCTG | | GGGCUGGU | | |
| 20 nt_3- | | 9_030 | CBE | GTGGGGA | | GGGG | | |
| 9_030_+_ | | | | GCA | | | | |
| 31946069_spCas9 | | | | | | | | |
| GuideDesign90_ | TATA | ABE_NGG_ | spCas9 | AATCTGT | 2893 | AAUCUGUG | 3199 | CGG |
| ABE_NGG_ | box | 20 nt_3- | IBE | GTTCTGG | | UUCUGGCA | | |
| 20 nt_3- | | 12_018 | | CACCTGC | | CCUG | | |
| 12_018_-_ | | | | GG | | | | |
| 31946040_spCas9 | | | | | | | | |
| GuideDesign9 | TATA | CBE_NGG_ | spCas9 | AATCTGT | 2894 | AAUCUGUG | 3200 | CGG |
| 0_CBE_NGG | box | 20 nt_4- | CBE | GTTCTGG | | UUCUGGCA | | |
| 20 nt_4- | | 9_003 | | CACCTGC | | CCUG | | |
| 9_003_-_ | | | | GG | | | | |
| 31946040_spCas9 | | | | | | | | |
| GuideDesign90_ | TATA | CBE_NGG_ | spCas9 | AATCTGT | 2895 | AAUCUGUG | 3201 | CGG |
| CBE_NGG_ | box | 20 nt_3- | NGG | GTTCTGG | | UUCUGGCA | | |
| 20 nt_3- | | 9_029 | CBE | CACCTGC | | CCUG | | |
| 9_029_-_ | | | | GG | | | | |
| 31946040_spCas9 | | | | | | | | |
| GuideDesign90_ | TATA | CBE_NGG_ | SpCas9 | AATCTGT | 2896 | AAUCUGUG | 3202 | CGG |
| CBE_NGG_ | box | 20 nt_3- | IBE | GTTCTGG | | UUCUGGCA | | |
| 20 nt_3- | | 16_033 | CBE | CACCTGC | | CCUG | | |
| 16_033_-_ | | | | GG | | | | |
| 31946040_SpCas9 | | | | | | | | |
| GuideDesign90_ | TATA | ABE_NG_ | spCas9 | AATCTGT | 2897 | AAUCUGUG | 3203 | CG |
| ABE_NG_20 nt_ | box | 20 nt 3- | NG | GTTCTGG | | UUCUGGCA | | |
| 3-9_027_-_ | | 9_027 | ABE | CACCTGC | | CCUG | | |
| 31946041_spCas9 | | | | G | | | | |
| GuideDesign90_ | TATA | CBE_NG_ | spCas9 | AATCTGT | 2898 | AAUCUGUG | 3204 | CG |
| CBE_NG_20 nt_ | box | 20 nt_4- | NG | GTTCTGG | | UUCUGGCA | | |
| 4-9_028_-_ | | 9_028 | CBE | CACCTGC | | CCUG | | |
| 31946041_spCas9 | | | | G | | | | |
| GuideDesign90_ | TATA | ABE_NRN_ | spCas9 | AATCTGT | 2899 | AAUCUGUG | 3205 | CGG |
| ABE_NRN_ | box | 20 nt_3- | NRN | GTTCTGG | | UUCUGGCA | | |
| 20 nt_3- | | 9_021 | ABE | CACCTGC | | CCUG | | |
| 9_021_-_ | | | | GG | | | | |
| 31946040_ | | | | | | | | |
| spCas9 | | | | | | | | |
| GuideDesign90_ | TATA | CBE_NRN_ | spCas9 | AATCTGT | 2900 | AAUCUGUG | 3206 | CGG |
| CBE_NRN_ | box | 20 nt_3- | NRN | GTTCTGG | | UUCUGGCA | | |
| 20 nt_3- | | 9_022 | CBE | CACCTGC | | CCUG | | |
| 9_022_-_ | | | | GG | | | | |
| 31946040_ | | | | | | | | |
| spCas9 | | | | | | | | |
| GuideDesign | TATA | MBE_NRN_ | spCas9 | AATCTGT | 2901 | AAUCUGUG | 3207 | CGG |
| 90_MBE_NRN_ | box | 20 nt_3- | NRN | GTTCTGG | | UUCUGGCA | | |
| 20 nt_3- | | 9_023 | MBE | CACCTGC | | CCUG | | |
| 9_023_-_ | | | | GG | | | | |
| 31946040_ | | | | | | | | |
| spCas9 | | | | | | | | |
| GuideDesign90_ | TATA | CBE_NRRH_ | spCas9 | AATCTGT | 2902 | AAUCUGUG | 3208 | CGGC |
| CBE_NRRH_ | box | 20 nt 3- | NRRH | GTTCTGG | | UUCUGGCA | | |
| 20 nt_3- | | 9_031 | CBE | CACCTGC | | CCUG | | |
| 9_031_-_ | | | | GGC | | | | |
| 31946039_ | | | | | | | | |
| spCas9 | | | | | | | | |
| GuideDesign90_ | TATA | ABE_NRRH_ | spCas9 | ACAATCT | 2903 | ACAAUCUG | 3209 | TGC |
| ABE_NGC_ | box | 20 nt_3- | NGC | GTGTTCT | | UGUUCUGG | | |
| 20 nt_3- | | 9_008 | ABE | GGCACCT | | CACC | | |
| 9_008_-_ | | | | GC | | | | |
| 31946042_ | | | | | | | | |
| spCas9 | | | | | | | | |
| GuideDesign90_ | TATA | ABE_NGC_ | spCas9 | ACAATCT | 2904 | ACAAUCUG | 3210 | TGC |
| ABE_NGC_ | box | 20 nt_3- | NGC | GTGTTCT | | UGUUCUGG | | |
| 20 nt_3- | | 12_020 | IBE | GGCACCT | | CACC | | |
| 12_020_-_ | | | | GC | | | | |
| 31946042_ | | | | | | | | |
| spCas9 | | | | | | | | |
| GuideDesign90_ | TATA | CBE_NGC_ | spCas9 | ACAATCT | 2905 | ACAAUCUG | 3211 | TGC |
| CBE_NGC_ | box | 20 nt_4- | NGC | GTGTTCT | | UGUUCUGG | | |
| 20 nt_4- | | 9_09 | CBE | GGCACCT | | CACC | | |
| 9_009_-_ | | | | GC | | | | |
| 31946042_ | | | | | | | | |
| spCas9 | | | | | | | | |
| GuideDesign90_ | TATA | ABE_NG_ | spCas9 | ACAATCT | 2906 | ACAAUCUG | 3212 | TG |
| ABE_NG_20 nt_ | box | 20 nt_3- | NG | GTGTTCT | | UGUUCUGG | | |
| 3-9_027_-_ | | 9_027 | ABE | GGCACCT | | CACC | | |
| 31946043_ | | | | G | | | | |
| spCas9 | | | | | | | | |
| Guide_Design90_ | TATA | CBE_NG_ | spCas9 | ACAATCT | 2907 | ACAAUCUG | 3213 | TG |
| CBE_NG_20 nt_ | box | 20 nt_4- | NG | GTGTTCT | | UGUUCUGG | | |
| 4-9_028_-_ | | 9_028 | CBE | GGCACCT | | CACC | | |
| 31946043_ | | | | G | | | | |
| spCas9 | | | | | | | | |
| Guide_Design90_ | TATA | ABE_NRN_ | spCas9 | ACAATCT | 2908 | ACAAUCUG | 3214 | TGC |
| ABE_NRN_ | box | 20 nt_3- | NRN | GTGTTCT | | UGUUCUGG | | |
| 20 nt_3- | | 9_021 | ABE | GGCACCT | | CACC | | |
| 9_021_-_ | | | | GC | | | | |
| 31946042_ | | | | | | | | |
| spCas9 | | | | | | | | |
| GuideDesign90_ | TATA | CBE_NRN_ | spCas9 | ACAATCT | 2909 | ACAAUCUG | 3215 | TGC |
| CBE_NRN_ | box | 20 nt_3- | NRN | GTGTTCT | | UGUUCUGG | | |
| 20 nt_3-_ | | 9_022 | CBE | GGCACCT | | CACC | | |
| 9_022_-_ | | | | GC | | | | |
| 31946042_ | | | | | | | | |
| spCas9 | | | | | | | | |
| GuideDesign90_ | TATA | MBE_NRN_ | spCas9 | ACAATCT | 2910 | ACAAUCUG | 3216 | TGC |
| MBE_NRN_ | box | 20 nt_3- | NRN | GTGTTCT | | UGUUCUGG | | |
| 20 nt_3- | | 9_023 | MBE | GGCACCT | | CACC | | |
| 9_023_-_ | | | | GC | | | | |
| 31946042_ | | | | | | | | |
| spCas9 | | | | | | | | |
| GuideDesign90_ | TATA | ABE_NGG_ | spCas9 | ACACAGA | 2911 | ACACAGAU | 3217 | TGG |
| ABE_NGG_ | box | 20 nt_3- | IBE | TTGTATA | | UGUAUAAA | | |
| 20 nt_3- | | 12_018 | | AAAGGCT | | AGGC | | |
| 12_018_+_ | | | | GG | | | | |
| 31946054_spCas9 | | | | | | | | |
| GuideDesign90_ | TATA | ABE_NGG_ | SpCas9 | ACACAGA | 2912 | ACACAGAU | 3218 | TGG |
| ABE_NGG_ | box | 20 nt_13- | IBE ex | TTGTATA | | UGUAUAAA | | |
| 20 nt_13- | | 16_032 | panded | AAAGGCT | | AGGC | | |
| 16_032_+_ | | | ABE | GG | | | | |
| 31946054_SpCas9 | | | | | | | | |
| GuideDesign90_ | TATA | CBE_NGG_ | spCas9 | ACACAGA | 2913 | ACACAGAU | 3219 | TGG |
| CBE_NGG_ | box | 20 nt_4- | CBE | TTGTATA | | UGUAUAAA | | |
| 20 nt_4- | | 9_003 | | AAAGGCT | | AGGC | | |
| 9_003_+_ | | | | GG | | | | |
| 31946054_spCas9 | | | | | | | | |
| GuideDesign90_ | TATA | CBE_NGG_ | spCas9 | ACACAGA | 2914 | ACACAGAU | 3220 | TGG |
| CBE_NGG_ | box | 20 nt_3- | NGG | TTGTATA | | UGUAUAAA | | |
| 20 nt_3- | | 9_029 | CBE | AAAGGCT | | AGGC | | |
| 9_029_+_ | | | | GG | | | | |
| 31946054_spCas9 | | | | | | | | |
| GuideDesign90_ | TATA | CBE_NGG_ | SpCas9 | ACACAGA | 2915 | ACACAGAU | 3221 | TGG |
| CBE_NGG_ | box | 20 nt_3- | IBE | TTGTATA | | UGUAUAAA | | |
| 20 nt_3- | | 16_033 | CBE | AAAGGCT | | AGGC | | |
| 16_033_+_ | | | | GG | | | | |
| 31946054_SpCas9 | | | | | | | | |
| GuideDesign90_ | TATA | ABE_NG_ | spCas9 | ACACAGA | 2916 | ACACAGAU | 3222 | TG |
| ABE_NG_ | box | 20 nt_3- | NG | TTGTATA | | UGUAUAAA | | |
| 20 nt_3- | | 9_027 | ABE | AAAGGCT | | AGGC | | |
| 9_027_+_ | | | | G | | | | |
| 31946054_spCas9 | | | | | | | | |
| GuideDesign90_ | TATA | CBE_NG_ | spCas9 | ACACAGA | 2917 | ACACAGAU | 3223 | TG |
| CBE_NG_ | box | 20 nt_4- | NG | TTGTATA | | UGUAUAAA | | |
| 20 nt_4- | | 9_028 | CBE | AAAGGCT | | AGGC | | |
| 9_028_+_ | | | | G | | | | |
| 31946054_spCas9 | | | | | | | | |
| GuideDesign90_ | TATA | ABE_NRN_ | spCas9 | ACACAGA | 2918 | ACACAGAU | 3224 | TGG |
| ABE_NRN_ | box | 20 nt_3- | NRN | TTGTATA | | UGUAUAAA | | |
| 20 nt_3- | | 9_021 | ABE | AAAGGCT | | AGGC | | |
| 9_021_+_ | | | | GG | | | | |
| 31946054_spCas9 | | | | | | | | |
| GuideDesign90_ | TATA | CBE_NRN_ | spCas9 | ACACAGA | 2919 | ACACAGAU | 3225 | TGG |
| CBE_NRN_ | box | 20 nt_3- | NRN | TTGTATA | | UGUAUAAA | | |
| 20 nt_3- | | 9_022 | CBE | AAAGGCT | | AGGC | | |
| 9_022_+_ | | | | GG | | | | |
| 31946054_spCas9 | | | | | | | | |
| GuideDesign90_ | TATA | MBE_NRN_ | spCas9 | ACACAGA | 2920 | ACACAGAU | 3226 | TGG |
| MBE_NRN_ | box | 20 nt_3- | NRN | TTGTATA | | UGUAUAAA | | |
| 20 nt_3- | | 9_023 | MBE | AAAGGCT | | AGGC | | |
| 9_023_+_ | | | | GG | | | | |
| 31946054_spCas9 | | | | | | | | |
| GuideDesign90_ | TATA | ABE_NGG_ | spCas9 | ACAGATT | 2921 | ACAGAUUG | 3227 | GGG |
| ABE_NGG_ | box | 20 nt_3- | IBE | GTATAAA | | UAUAAAAG | | |
| 20nt_3- | | 12_018 | | AGGCTGG | | GCUG | | |
| 12_018_+_ | | | | GG | | | | |
| 31946056 spCas9 | | | | | | | | |
| GuideDesign90_ | TATA | ABE_NGG_ | SpCas9 | ACAGATT | 2922 | ACAGAUUG | 3228 | GGG |
| ABE_NGG_ | box | 20 nt_13- | IBE_ | GTATAAA | | UAUAAAAG | | |
| 20 nt_13- | | 16_032 | expanded | AGGCTGG | | GCUG | | |
| 16_032_+_ | | | ABE | GG | | | | |
| 31946056_SpCas9 | | | | | | | | |
| GuideDesign90_ | TATA | CBE_NGG_ | spCas9 | ACAGATT | 2923 | ACAGAUUG | 3229 | GGG |
| CBE_NGG_ | box | 20 nt_4- | CBE | GTATAAA | | UAUAAAAG | | |
| 20 nt_4- | | 9_003 | | AGGCTGG | | GCUG | | |
| 9_003_+_ | | | | GG | | | | |
| 31946056_spCas9 | | | | | | | | |
| GuideDesign90_ | TATA | CBE_NGG_ | spCas9 | ACAGATT | 2924 | ACAGAUUG | 3230 | GGG |
| CBE_NGG | box | 20 nt_3- | NGG | GTATAAA | | UAUAAAAG | | |
| 20 nt_3- | | 9_029 | CBE | AGGCTGG | | GCUG | | |
| 9_029_+_ | | | | GG | | | | |
| 31946056_spCas9 | | | | | | | | |
| GuideDesign90_ | TATA | CBE_NGG_ | SpCas9 | ACAGATT | 2925 | ACAGAUUG | 3231 | GGG |
| CBE_NGG_ | box | 20 nt_3- | IBE | GTATAAA | | UAUAAAAG | | |
| 20 nt_3- | | 16_033 | CBE | AGGCTGG | | GCUG | | |
| 16_033_+_ | | | | GG | | | | |
| 31946056_SpCas9 | | | | | | | | |
| GuideDesign90_ | TATA | ABE_NG_ | spCas9 | ACAGATT | 2926 | ACAGAUUG | 3232 | GG |
| ABE_NG_ | box | 20 nt_3- | NG | GTATAAA | | UAUAAAAG | | |
| 20 nt_3- | | 9_027 | ABE | AGGCTGG | | GCUG | | |
| 9_027_+_ | | | | G | | | | |
| 31946056_spCas9 | | | | | | | | |
| GuideDesign90_ | TATA | CBE_NG_ | spCas9 | ACAGATT | 2927 | ACAGAUUG | 3233 | GG |
| CBE_NG_ | box | 20 nt_4- | NG | GTATAAA | | UAUAAAAG | | |
| 20 nt_4- | | 9_028 | CBE | AGGCTGG | | GCUG | | |
| 9_028_+_ | | | | G | | | | |
| 31946056_spCas9 | | | | | | | | |
| GuideDesign90_ | TATA | ABE_NRN_ | spCas9 | ACAGATT | 2928 | ACAGAUUG | 3234 | GGG |
| ABE_NRN_ | box | 20 nt_3- | NRN | GTATAAA | | UAUAAAAG | | |
| 20 nt_3- | | 9_021 | ABE | AGGCTGG | | GCUG | | |
| 9_021_+_ | | | | GG | | | | |
| 31946056_spCas9 | | | | | | | | |
| GuideDesign90_ | TATA | CBE_NRN_ | spCas9 | ACAGATT | 2929 | ACAGAUUG | 3235 | GGG |
| CBE_NRN_ | box | 20 nt_3- | NRN | GTATAAA | | UAUAAAAG | | |
| 20 nt_3- | | 9_022 | CBE | AGGCTGG | | GCUG | | |
| 9_022_+_ | | | | GG | | | | |
| 31946056_spCas9 | | | | | | | | |
| GuideDesign90_ | TATA | MBE_NRN_ | spCas9 | ACAGATT | 2930 | ACAGAUUG | 3236 | GGG |
| MBE_NRN_ | box | 20 nt_3- | NRN | GTATAAA | | UAUAAAAG | | |
| 20 nt_3- | | 9_023 | MBE | AGGCTGG | | GCUG | | |
| 9_023_+_ | | | | GG | | | | |
| 31946056_spCas9 | | | | | | | | |
| GuideDesign90_ | TATA | ABE_NGC_ | spCas9 | AGAACAC | 2931 | AGAACACA | 3237 | GGC |
| ABE_NGC_ | box | 20 nt_3- | NGC | AGATTGT | | GAUUGUAU | | |
| 20 nt_3- | | 12_020 | IBE | ATAAAAG | | AAAA | | |
| 12_020_+_ | | | | GC | | | | |
| 31946051_spCas9 | | | | | | | | |
| GuideDesign90_ | TATA | CBE_NGC_ | spCas9 | AGAACAC | 2932 | AGAACACA | 3238 | GGC |
| CBE_NGC_ | box | 20 nt_4- | NGC | AGATTGT | | GAUUGUAU | | |
| 20 nt_4- | | 9_009 | CBE | ATAAAAG | | AAAA | | |
| 9_009_+_ | | | | GC | | | | |
| 31946051_spCas9 | | | | | | | | |
| GuideDesign90_ | TATA | ABE_NG_ | spCas9 | AGAACAC | 2933 | AGAACACA | 3239 | GG |
| ABE_NG_ | box | 20 nt_3- | NG | AGATTGT | | GAUUGUAU | | |
| 20 nt_3- | | 9_027 | ABE | ATAAAAG | | AAAA | | |
| 9_027_+_ | | | | G | | | | |
| 31946051_spCas9 | | | | | | | | |
| GuideDesign90_ | TATA | CBE_NG_ | spCas9 | AGAACAC | 2934 | AGAACACA | 3240 | GG |
| CBE_NG_ | box | 20 nt_4- | NG | AGATTGT | | GAUUGUAU | | |
| 20 nt_4- | | 9_028 | CBE | ATAAAAG | | AAAA | | |
| 9_028_+_ | | | | G | | | | |
| 31946051_spCas9 | | | | | | | | |
| GuideDesign90_ | TATA | ABE_NRN_ | spCas9 | AGAACAC | 2935 | AGAACACA | 3241 | GGC |
| ABE_NRN_ | box | 20 nt_3- | NRN | AGATTGT | | GAUUGUAU | | |
| 20 nt_3- | | 9_021 | ABE | ATAAAAG | | AAAA | | |
| 9_021_+_ | | | | GC | | | | |
| 31946051_spCas9 | | | | | | | | |
| GuideDesign90_ | TATA | CBE_NRN_ | spCas9 | AGAACAC | 2936 | AGAACACA | 3242 | GGC |
| CBE_NRN_ | box | 20 nt_3- | NRN | AGATTGT | | GAUUGUAU | | |
| 20 nt_3- | | 9_022 | CBE | ATAAAAG | | AAAA | | |
| 9_022_+_ | | | | GC | | | | |
| 31946051_spCas9 | | | | | | | | |
| GuideDesign90_ | TATA | MBE_NRN_ | spCas9 | AGAACAC | 2937 | AGAACACA | 3243 | GGC |
| MBE_NRN_ | box | 20 nt_3- | NRN | AGATTGT | | GAUUGUAU | | |
| 20 nt_3- | | 9_023 | MBE | ATAAAAG | | AAAA | | |
| 9_023_+_ | | | | GC | | | | |
| 31946051_spCas9 | | | | | | | | |
| GuideDesign90_ | TATA | ABE_NRCH_ | spCas9 | AGAACAC | 2938 | AGAACACA | 3244 | GGCT |
| ABE_NRC_ | box | 20 nt_3- | NRCH | AGATTGT | | GAUUGUAU | | |
| H_20 nt_3- | | 9_024 | ABE | ATAAAAG | | AAAA | | |
| 9_024_+_ | | | | GCT | | | | |
| 31946051_spCas9 | | | | | | | | |
| GuideDesign90_ | TATA | CBE_NRCH_ | spCas9 | AGAACAC | 2939 | AGAACACA | 3245 | GGCT |
| CBE_NRC_ | box | 20 nt_3- | NRCH | AGATTGT | | GAUUGUAU | | |
| H_20 nt_3- | | 9_030 | CBE | ATAAAAG | | AAAA | | |
| 9_030_+_ | | | | GCT | | | | |
| 31946051_spCas9 | | | | | | | | |
| GuideDesign90_ | TATA | ABE_NGC_ | spCas9 | AGATTGT | 2940 | AGAUUGUA | 3246 | GGC |
| ABE_NGC_ | box | 20 nt_3- | NGC | ATAAAAG | | UAAAAGGC | | |
| 20 nt_3- | | 9_008 | ABE | GCTGGGG | | UGGG | | |
| 9_008_+_ | | | | GC | | | | |
| 31946058_spCas9 | | | | | | | | |
| GuideDesign90_ | TATA | ABE_NGC_ | spCas9 | AGATTGT | 2941 | AGAUUGUA | 3247 | GGC |
| ABE_NGC_ | box | 20 nt_3- | NGC | ATAAAAG | | UAAAAGGC | | |
| 20 nt_3- | | 12_020 | IBE | | | UGGG | | |
| 12_020_+_ | | | | GCTGGGG | | | | |
| 31946058_spCas9 | | | | GC | | | | |
| GuideDesign90_ | TATA | CBE_NGC_ | spCas9 | AGATTGT | 2942 | AGAUUGUA | 3248 | GGC |
| CBE_NGC_ | box | 20 nt_4- | NGC | ATAAAAG | | UAAAAGGC | | |
| 20 nt_4- | | 9_009 | CBE | GCTGGGG | | UGGG | | |
| 9_009_+_ | | | | GC | | | | |
| 31946058_spCas9 | | | | | | | | |
| GuideDesign90_ | TATA | ABE_NG_ | spCas9 | AGATTGT | 2943 | AGAUUGUA | 3249 | GG |
| ABE_NG_ | box | 20 nt_3- | NG | ATAAAAG | | UAAAAGGC | | |
| 20 nt_3- | | 9_027 | ABE | GCTGGGG | | UGGG | | |
| 9_027_+_ | | | | G | | | | |
| 31946058_spCas9 | | | | | | | | |
| GuideDesign90_ | TATA | CBE_NG_ | spCas9 | AGATTGT | 2944 | AGAUUGUA | 3250 | GG |
| CBE_NG_ | box | 20 nt_4- | NG | ATAAAAG | | UAAAAGGC | | |
| 20 nt_4- | | 9_028 | CBE | GCTGGGG | | UGGG | | |
| 9_028_+_ | | | | G | | | | |
| 31946058_spCas9 | | | | | | | | |
| GuideDesign90_ | TATA | ABE_NRN_ | spCas9 | AGATTGT | 2945 | AGAUUGUA | 3251 | GGC |
| ABE_NRN_ | box | 20 nt_3- | NRN | ATAAAAG | | UAAAAGGC | | |
| 20 nt_3- | | 9_021 | ABE | GCTGGGG | | UGGG | | |
| 9_021_+_ | | | | GC | | | | |
| 31946058_spCas9 | | | | | | | | |
| GuideDesign90_ | TATA | CBE_NRN_ | spCas9 | AGATTGT | 2946 | AGAUUGUA | 3252 | GGC |
| CBE_NRN_ | box | 20 nt_3- | NRN | ATAAAAG | | UAAAAGGC | | |
| 20 nt_3- | | 9_022 | CBE | GCTGGGG | | UGGG | | |
| 9_022_+_ | | | | GC | | | | |
| 31946058_spCas9 | | | | | | | | |
| GuideDesign90_ | TATA | MBE_NRN_ | spCas9 | AGATTGT | 2947 | AGAUUGUA | 3253 | GGC |
| MBE_NRN_ | box | 20 nt_3- | NRN | ATAAAAG | | UAAAAGGC | | |
| 20 nt_3- | | 9_023 | MBE | GCTGGGG | | UGGG | | |
| 9_023_+_ | | | | GC | | | | |
| 31946058_spCas9 | | | | | | | | |
| GuideDesign90_ | TATA | ABE_NRCH_ | spCas9 | AGATTGT | 2948 | AGAUUGUA | 3254 | GGCT |
| ABE_NRC_ | box | 20 nt_3- | NRCH | ATAAAAG | | UAAAAGGC | | |
| H_20 nt_3- | | 9_024 | ABE | GCTGGGG | | UGGG | | |
| 9_024_+_ | | | | GCT | | | | |
| 31946058_spCas9 | | | | | | | | |
| GuideDesign90_ | TATA | CBE_NRCH_ | spCas9 | AGATTGT | 2949 | AGAUUGUA | 3255 | GGCT |
| CBE_NRC_ | box | 20 nt_3- | NRCH | ATAAAAG | | UAAAAGGC | | |
| H_20 nt_3- | | 9_030 | CBE | GCTGGGG | | UGGG | | |
| 9_030_+_ | | | | GCT | | | | |
| 31946058_spCas9 | | | | | | | | |
| GuideDesign90_ | TATA | CBE_ | saCas9 | AGATTGT | 2950 | AGAUUGUA | 3256 | GCTGG |
| CBE_NNNRRT_ | box | NNNRRT_ | KKH | ATAAAAG | | UAAAAGGC | | T |
| 21 nt_3- | | 21 nt_3- | CBE | GCTGGGG | | UGGGG | | |
| 12_015_+_ | | 12_015 | | GCTGGT | | | | |
| 31946058_saCas9 | | | | | | | | |
| GuideDesign90_ | TATA | ABE_NGG_ | spCas9 | ATAAAAG | 2951 | AUAAAAGG | 3257 | GGG |
| ABE_NGG_ | box | 20 nt_3- | IBE | GCTGGGG | | CUGGGGGC | | |
| 20 nt_3- | | 12_018 | | GCTGGTG | | UGGU | | |
| 12_018_+_ | | | | GG | | | | |
| 31946065_spCas9 | | | | | | | | |
| GuideDesign90_ | TATA | CBE_NGG_ | spCas9 | ATAAAAG | 2952 | AUAAAAGG | 3258 | GGG |
| CBE_NGG_ | box | 20 nt_4- | CBE | GCTGGGG | | CUGGGGGC | | |
| 20 nt_4- | | 9_003 | | GCTGGTG | | UGGU | | |
| 9_003_+_ | | | | GG | | | | |
| 31946065_spCas9 | | | | | | | | |
| GuideDesign90_ | TATA | CBE_NGG_ | spCas9 | ATAAAAG | 2953 | AUAAAAGG | 3259 | GGG |
| CBE_NGG_ | box | 20 nt_3- | NGG | GCTGGGG | | CUGGGGGC | | |
| 20 nt_3- | | 9_029 | CBE | GCTGGTG | | UGGU | | |
| 9_029_+_ | | | | GG | | | | |
| 31946065_spCas9 | | | | | | | | |
| GuideDesign90_ | TATA | CBE_NGG_ | SpCas9 | ATAAAAG | 2954 | AUAAAAGG | 3260 | GGG |
| CBE_NGG_ | box | 20 nt_3- | IBE | GCTGGGG | | CUGGGGGC | | |
| 20 nt_3- | | 16_033 | CBE | GCTGGTG | | UGGU | | |
| 16_033_+_ | | | | GG | | | | |
| 31946065_SpCas9 | | | | | | | | |
| GuideDesign90_ | TATA | ABE_NG_ | spCas9 | ATAAAAG | 2955 | AUAAAAGG | 3261 | GG |
| ABE_NG_ | box | 20 nt_3- | NG | GCTGGGG | | CUGGGGGC | | |
| 20 nt_3- | | 9_027 | ABE | GCTGGTG | | UGGU | | |
| 9_027_+_ | | | | G | | | | |
| 31946065_spCas9 | | | | | | | | |
| GuideDesign90_ | TATA | CBE_NG_ | spCas9 | ATAAAAG | 2956 | AUAAAAGG | 3262 | GG |
| CBE_NG_ | box | 20 nt_4- | NG | GCTGGGG | | CUGGGGGC | | |
| 20 nt_4- | | 9_028 | CBE | GCTGGTG | | UGGU | | |
| 9_028_+_ | | | | G | | | | |
| 31946065_spCas9 | | | | | | | | |
| GuideDesign90_ | TATA | ABE_NRN_ | spCas9 | ATAAAAG | 2957 | AUAAAAGG | 3263 | GGG |
| ABE_NRN_ | box | 20 nt_3- | NRN | GCTGGGG | | CUGGGGGC | | |
| 20 nt_3- | | 9_021 | ABE | GCTGGTG | | UGGU | | |
| 9_021_+_ | | | | GG | | | | |
| 31946065_spCas9 | | | | | | | | |
| GuideDesign90_ | TATA | CBE_NRN_ | spCas9 | ATAAAAG | 2958 | AUAAAAGG | 3264 | GGG |
| CBE_NRN_ | box | 20 nt_3- | NRN | GCTGGGG | | CUGGGGGC | | |
| 20 nt_3- | | 9_022 | CBE | GCTGGTG | | UGGU | | |
| 9_022_+_ | | | | GG | | | | |
| 31946065_spCas9 | | | | | | | | |
| GuideDesign90_ | TATA | MBE_NRN_ | spCas9 | ATAAAAG | 2959 | AUAAAAGG | 3265 | GGG |
| MBE_NRN_ | box | 20 nt_3- | NRN | GCTGGGG | | CUGGGGGC | | |
| 20 nt_3- | | 9_023 | MBE | GCTGGTG | | UGGU | | |
| 9_023_+_ | | | | GG | | | | |
| 31946065_spCas9 | | | | | | | | |
| GuideDesign90_ | TATA | ABE_NGC_ | spCas9 | ATCTGTG | 2960 | AUCUGUGU | 3266 | GGC |
| ABE_NGC | box | 20 nt_3- | NGC | TTCTGGC | | UCUGGCAC | | |
| 20 nt_3- | | 12_020 | IBE | ACCTGCG | | CUGC | | |
| 12_020_-_ | | | | GC | | | | |
| 31946039_ | | | | | | | | |
| spCas9 | | | | | | | | |
| GuideDesign90_ | TATA | CBE_NGC_ | spCas9 | ATCTGTG | 2961 | AUCUGUGU | 3267 | GGC |
| CBE_NGC_ | box | 20 nt_4- | NGC | TTCTGGC | | UCUGGCAC | | |
| 20 nt_4- | | 9_009 | CBE | ACCTGCG | | CUGC | | |
| 9_009_-_ | | | | GC | | | | |
| 31946039_ | | | | | | | | |
| spCas9 | | | | | | | | |
| GuideDesign90_ | TATA | ABE_NG_ | spCas9 | ATCTGTG | 2962 | AUCUGUGU | 3268 | GG |
| ABE_NG_20 nt_ | box | 20 nt_3- | NG | TTCTGGC | | UCUGGCAC | | |
| 3-9_027_-_ | | 9_027 | ABE | ACCTGCG | | CUGC | | |
| 31946040_sp | | | | G | | | | |
| Cas9 | | | | | | | | |
| GuideDesign90_ | TATA | CBE_NG_ | spCas9 | ATCTGTG | 2963 | AUCUGUGU | 3269 | GG |
| CBE_NG_20 nt_ | box | 20 nt_4- | NG | TTCTGGC | | UCUGGCAC | | |
| 4-9_028_-_ | | 9_028 | CBE | ACCTGCG | | CUGC | | |
| 31946040_ | | | | G | | | | |
| spCas9 | | | | | | | | |
| GuideDesign90_ | TATA | ABE_NRN_ | spCas9 | ATCTGTG | 2964 | AUCUGUGU | 3270 | GGC |
| ABE_NRN_ | box | 20 nt_3- | NRN | TTCTGGC | | UCUGGCAC | | |
| 20 nt_3- | | 9_021 | ABE | ACCTGCG | | CUGC | | |
| 9_021_-_ | | | | GC | | | | |
| 31946039_ | | | | | | | | |
| spCas9 | | | | | | | | |
| GuideDesign90_ | TATA | CBE_NRN_ | spCas9 | ATCTGTG | 2965 | AUCUGUGU | 3271 | GGC |
| CBE_NRN_ | box | 20 nt_3- | NRN | TTCTGGC | | UCUGGCAC | | |
| 20 nt_3- | | 9_022 | CBE | ACCTGCG | | CUGC | | |
| 9_022_-_ | | | | GC | | | | |
| 31946039_ | | | | | | | | |
| spCas9 | | | | | | | | |
| GuideDesign90_ | TATA | MBE_NRN_ | spCas9 | ATCTGTG | 2966 | AUCUGUGU | 3272 | GGC |
| MBE_NRN_ | box | 20 nt_3- | NRN | TTCTGGC | | UCUGGCAC | | |
| 20 nt_3- | | 9_023 | MBE | ACCTGCG | | CUGC | | |
| 9_023_-_ | | | | GC | | | | |
| 31946039_ | | | | | | | | |
| spCas9 | | | | | | | | |
| GuideDesign90_ | TATA | ABE_NRCH_ | spCas9 | ATCTGTG | 2967 | AUCUGUGU | 3273 | GGCT |
| ABE_NRCH_ | box | 20 nt_3- | NRCH | TTCTGGC | | UCUGGCAC | | |
| 20 nt_3- | | 9_024 | ABE | ACCTGCG | | CUGC | | |
| 9_024_-_ | | | | GCT | | | | |
| 31946038_ | | | | | | | | |
| spCas9 | | | | | | | | |
| GuideDesign90_ | TATA | CBE_NRCH_ | spCas9 | ATCTGTG | 2968 | AUCUGUGU | 3274 | GGCT |
| CBE_NRCH_ | box | 20 nt_3- | NRCH | TTCTGGC | | UCUGGCAC | | |
| 20 nt_3- | | 9_030 | CBE | ACCTGCG | | CUGC | | |
| 9_030_-_ | | | | GCT | | | | |
| 31946038_ | | | | | | | | |
| spCas9 | | | | | | | | |
| GuideDesign90_ | TATA | ABE_NGG_ | spCas9 | CACAGAT | 2969 | CACAGAUU | 3275 | GGG |
| ABE_NGG_ | box | 20 nt_3- | IBE | TGTATAA | | GUAUAAAA | | |
| 20 nt_3- | | 12_018 | | AAGGCTG | | GGCU | | |
| 12_018_+_ | | | | GG | | | | |
| 31946055_spCas9 | | | | | | | | |
| Guide_Design90_ | TATA | ABE_NGG_ | SpCas9 | CACAGAT | 2970 | CACAGAUU | 3276 | GGG |
| ABE_NGG_ | box | 20 nt_13- | IBE_ | TGTATAA | | GUAUAAAA | | |
| 20 nt_13- | | 16_032 | expanded | AAGGCTG | | GGCU | | |
| 16_032_+_ | | | ABE | GG | | | | |
| 31946055_SpCas9 | | | | | | | | |
| GuideDesign90_ | TATA | CBE_NGG_ | spCas9 | CACAGAT | 2971 | CACAGAUU | 3277 | GGG |
| CBE_NGG_ | box | 20 nt_4- | CBE | TGTATAA | | GUAUAAAA | | |
| 20 nt_4- | | 9_003 | | AAGGCTG | | GGCU | | |
| 9_003_+_ | | | | GG | | | | |
| 31946055_spCas9 | | | | | | | | |
| GuideDesign90_ | TATA | CBE_NGG_ | spCas9 | CACAGAT | 2972 | CACAGAUU | 3278 | GGG |
| CBE_NGG_ | box | 20 nt_3- | NGG | TGTATAA | | GUAUAAAA | | |
| 20 nt_3- | | 9_029 | CBE | AAGGCTG | | GGCU | | |
| 9_029_+_ | | | | GG | | | | |
| 31946055_spCas9 | | | | | | | | |
| GuideDesign90_ | TATA | CBE_NGG_ | SpCas9 | CACAGAT | 2973 | CACAGAUU | 3279 | GGG |
| CBE_NGG_ | box | 20 nt_3- | IBE | TGTATAA | | GUAUAAAA | | |
| 20 nt_3- | | 16_033 | CBE | AAGGCTG | | GGCU | | |
| 16_033_+_ | | | | GG | | | | |
| 31946055_SpCas9 | | | | | | | | |
| GuideDesign90_ | TATA | ABE_NG_ | spCas9 | CACAGAT | 2974 | CACAGAUU | 3280 | GG |
| ABE_NG_ | box | 20 nt_3- | NG | TGTATAA | | GUAUAAAA | | |
| 20 nt_3- | | 9_027 | ABE | AAGGCTG | | GGCU | | |
| 9_027_+_ | | | | G | | | | |
| 31946055_spCas9 | | | | | | | | |
| GuideDesign90_ | TATA | CBE_NG_ | spCas9 | CACAGAT | 2975 | CACAGAUU | 3281 | GG |
| CBE_NG_ | box | 20 nt_4- | NG | TGTATAA | | GUAUAAAA | | |
| 20 nt_4- | | 9_028 | CBE | AAGGCTG | | GGCU | | |
| 9_028_+_ | | | | G | | | | |
| 31946055_spCas9 | | | | | | | | |
| GuideDesign90_ | TATA | ABE_NRN_ | spCas9 | CACAGAT | 2976 | CACAGAUU | 3282 | GGG |
| ABE_NRN_ | box | 20 nt_3- | NRN | TGTATAA | | GUAUAAAA | | |
| 20 nt_3- | | 9_021 | ABE | AAGGCTG | | GGCU | | |
| 9_021_+_ | | | | GG | | | | |
| 31946055_spCas9 | | | | | | | | |
| GuideDesign90_ | TATA | CBE_NRN_ | spCas9 | CACAGAT | 2977 | CACAGAUU | 3283 | GGG |
| CBE_NRN_ | box | 20 nt_3- | NRN | TGTATAA | | GUAUAAAA | | |
| 20 nt_3- | | 9_022 | CBE | AAGGCTG | | GGCU | | |
| 9_022_+_ | | | | GG | | | | |
| 31946055_spCas9 | | | | | | | | |
| GuideDesign90_ | TATA | MBE_NRN_ | spCas9 | CACAGAT | 2978 | CACAGAUU | 3284 | GGG |
| MBE_NRN_ | box | 20 nt_3- | NRN | TGTATAA | | GUAUAAAA | | |
| 20 nt_3- | | 9_023 | MBE | AAGGCTG | | GGCU | | |
| 9_023_+_ | | | | GG | | | | |
| 31946055_spCas9 | | | | | | | | |
| GuideDesign90_ | TATA | ABE_NGG_ | spCas9 | CAGAACA | 2979 | CAGAACAC | 3285 | AGG |
| ABE_NGG_ | box | 20 nt_3- | IBE | CAGATTG | | AGAUUGUA | | |
| 20 nt_3- | | 12_018 | | TATAAAA | | UAAA | | |
| 12_018_+_ | | | | GG | | | | |
| 31946050_spCas9 | | | | | | | | |
| GuideDesign90_ | TATA | ABE_NGG_ | SpCas9 | CAGAACA | 2980 | CAGAACAC | 3286 | AGG |
| ABE_NGG_ | box | 20 nt_13- | IBE_ | CAGATTG | | AGAUUGUA | | |
| 20 nt_13- | | 16_032 | expanded | TATAAAA | | UAAA | | |
| 16_032_+_ | | | ABE | GG | | | | |
| 31946050_SpCas9 | | | | | | | | |
| GuideDesign90_ | TATA | CBE_NGG_ | spCas9 | CAGAACA | 2981 | CAGAACAC | 3287 | AGG |
| CBE_NGG_ | box | 20 nt_4- | CBE | CAGATTG | | AGAUUGUA | | |
| 20 nt_4- | | 9_003 | | TATAAAA | | UAAA | | |
| 9_003_+_ | | | | GG | | | | |
| 31946050_spCas9 | | | | | | | | |
| GuideDesign90_ | TATA | CBE_NGG_ | spCas9 | CAGAACA | 2982 | CAGAACAC | 3288 | AGG |
| CBE_NGG_ | box | 20 nt_3- | NGG | CAGATTG | | AGAUUGUA | | |
| 20 nt_3- | | 9_029 | CBE | TATAAAA | | UAAA | | |
| 9_029_+_ | | | | GG | | | | |
| 31946050_spCas9 | | | | | | | | |
| GuideDesign90_ | TATA | CBE_NGG_ | SpCas9 | CAGAACA | 2983 | CAGAACAC | 3289 | AGG |
| CBE_NGG_ | box | 20 nt_3- | IBE | CAGATTG | | AGAUUGUA | | |
| 20 nt_3- | | 16_033 | CBE | TATAAAA | | UAAA | | |
| 16_033_+_ | | | | GG | | | | |
| 31946050_SpCas9 | | | | | | | | |
| GuideDesign90_ | TATA | ABE_NG_ | spCas9 | CAGAACA | 2984 | CAGAACAC | 3290 | AG |
| ABE_NG_ | box | 20 nt_3- | NG | CAGATTG | | AGAUUGUA | | |
| 20 nt_3- | | 9_027 | ABE | TATAAAA | | UAAA | | |
| 9_027_+_ | | | | G | | | | |
| 31946050_spCas9 | | | | | | | | |
| GuideDesign90_ | TATA | CBE_NG_ | spCas9 | CAGAACA | 2985 | CAGAACAC | 3291 | AG |
| CBE_NG_ | box | 20 nt_4- | NG | CAGATTG | | AGAUUGUA | | |
| 20 nt_4- | | 9_028 | CBE | TATAAAA | | UAAA | | |
| 9_028_+_ | | | | G | | | | |
| 31946050_spCas9 | | | | | | | | |
| GuideDesign90_ | TATA | ABE_NRN_ | spCas9 | CAGAACA | 2986 | CAGAACAC | 3292 | AGG |
| ABE_NRN_ | box | 20 nt_3- | NRN | CAGATTG | | AGAUUGUA | | |
| 20 nt_3- | | 9_021 | ABE | TATAAAA | | UAAA | | |
| 9_021_+_ | | | | GG | | | | |
| 31946050_spCas9 | | | | | | | | |
| GuideDesign90_ | TATA | CBE_NRN_ | spCas9 | CAGAACA | 2987 | CAGAACAC | 3293 | AGG |
| CBE_NRN_ | box | 20 nt_3- | NRN | CAGATTG | | AGAUUGUA | | |
| 20 nt_3- | | 9_022 | CBE | TATAAAA | | UAAA | | |
| 9_022_+_ | | | | GG | | | | |
| 31946050_spCas9 | | | | | | | | |
| GuideDesign90_ | TATA | MBE_NRN_ | spCas9 | CAGAACA | 2988 | CAGAACAC | 3294 | AGG |
| MBE_NRN_ | box | 20 nt_3- | NRN | CAGATTG | | AGAUUGUA | | |
| 20 nt_3- | | 9_023 | MBE | TATAAAA | | UAAA | | |
| 9_023_+_ | | | | GG | | | | |
| 31946050_spCas9 | | | | | | | | |
| GuideDesign90_ | TATA | CBE_NRRH_ | spCas9 | CAGAACA | 2989 | CAGAACAC | 3295 | AGGC |
| CBE_NRR_ | box | 20 nt_3- | NRRH | CAGATTG | | AGAUUGUA | | |
| H_20 nt_3- | | 9_031 | CBE | TATAAAA | | UAAA | | |
| 9_031_+_ | | | | GGC | | | | |
| 31946050_spCas9 | | | | | | | | |
| GuideDesign90_ | TATA | ABE_NGG_ | spCas9 | CAGATTG | 2990 | CAGAUUGU | 3296 | GGG |
| ABE_NGG_ | box | 20 nt_3- | IBE | TATAAAA | | AUAAAAGG | | |
| 20 nt_3- | | 12_018 | | GGCTGGG | | CUGG | | |
| 12_018_+_ | | | | GG | | | | |
| 31946057_spCas9 | | | | | | | | |
| GuideDesign90_ | TATA | ABE_NGG_ | SpCas9 | CAGATTG | 2991 | CAGAUUGU | 3297 | GGG |
| ABE_NGG_ | box | 20 nt_13- | IBE_ | TATAAAA | | AUAAAAGG | | |
| 20 nt_13- | | 16_032 | expanded | GGCTGGG | | CUGG | | |
| 16_032_+_ | | | ABE | GG | | | | |
| 31946057_SpCas9 | | | | | | | | |
| GuideDesign90_ | TATA | CBE_NGG_ | spCas9 | CAGATTG | 2992 | CAGAUUGU | 3298 | GGG |
| CBE_NGG_ | box | 20 nt_4- | CBE | TATAAAA | | AUAAAAGG | | |
| 20 nt_4- | | 9_003 | | GGCTGGG | | CUGG | | |
| 9_003_+_ | | | | GG | | | | |
| 31946057_spCas9 | | | | | | | | |
| GuideDesign90_ | TATA | CBE_NGG_ | spCas9 | CAGATTG | 2993 | CAGAUUGU | 3299 | GGG |
| CBE_NGG | box | 20 nt_3- | NGG | TATAAAA | | AUAAAAGG | | |
| 20 nt_3- | | 9_029 | CBE | GGCTGGG | | CUGG | | |
| 9_029_+_ | | | | GG | | | | |
| 31946057_spCas9 | | | | | | | | |
| GuideDesign90_ | TATA | CBE_NGG_ | SpCas9 | CAGATTG | 2994 | CAGAUUGU | 3300 | GGG |
| CBE_NGG_ | box | 20 nt_3- | IBE | TATAAAA | | AUAAAAGG | | |
| 20 nt_3- | | 16_033 | CBE | GGCTGGG | | CUGG | | |
| 16_033_+_ | | | | GG | | | | |
| 31946057_SpCas9 | | | | | | | | |
| GuideDesign90_ | TATA | ABE_NG_ | spCas9 | CAGATTG | 2995 | CAGAUUGU | 3301 | GG |
| ABE_NG_ | box | 20 nt_3- | NG | TATAAAA | | AUAAAAGG | | |
| 20 nt_3- | | 9_027 | ABE | GGCTGGG | | CUGG | | |
| 9_027_+_ | | | | G | | | | |
| 31946057_spCas9 | | | | | | | | |
| GuideDesign90_ | TATA | CBE_NG_ | spCas9 | CAGATTG | 2996 | CAGAUUGU | 3302 | GG |
| CBE_NG_ | box | 20 nt_4- | NG | TATAAAA | | AUAAAAGG | | |
| 20 nt_4- | | 9_028 | CBE | GGCTGGG | | CUGG | | |
| 9_028_+_ | | | | G | | | | |
| 31946057_spCas9 | | | | | | | | |
| GuideDesign90_ | TATA | ABE_NRN_ | spCas9 | CAGATTG | 2997 | CAGAUUGU | 3303 | GGG |
| ABE_NRN_ | box | 20 nt_3- | NRN | TATAAAA | | AUAAAAGG | | |
| 20 nt_3- | | 9_021 | ABE | GGCTGGG | | CUGG | | |
| 9_021_+_ | | | | GG | | | | |
| 31946057_spCas9 | | | | | | | | |
| GuideDesign90_ | TATA | CBE_NRN_ | spCas9 | CAGATTG | 2998 | CAGAUUGU | 3304 | GGG |
| CBE_NRN_ | box | 20 nt_3- | NRN | TATAAAA | | AUAAAAGG | | |
| 20 nt_3- | | 9_022 | CBE | GGCTGGG | | CUGG | | |
| 9_022_+_ | | | | GG | | | | |
| 31946057_spCas9 | | | | | | | | |
| GuideDesign90_ | TATA | MBE_NRN_ | spCas9 | CAGATTG | 2999 | CAGAUUGU | 3305 | GGG |
| MBE_NRN_ | box | 20 nt_3- | NRN | TATAAAA | | AUAAAAGG | | |
| 20 nt_3- | | 9_023 | MBE | | | CUGG | | |
| 9_023_+_ | | | | GGCTGGG | | | | |
| 31946057_spCas9 | | | | GG | | | | |
| GuideDesign90_ | TATA | CBE_NRRH_ | spCas9 | CAGATTG | 3000 | CAGAUUGU | 3306 | GGGC |
| CBE_NRRH_ | box | 20 nt_3- | NRRH | TATAAAA | | AUAAAAGG | | |
| 20 nt_3- | | 9_031 | CBE | GGCTGGG | | CUGG | | |
| 9_031_+_ | | | | GGC | | | | |
| 31946057_spCas9 | | | | | | | | |
| GuideDesign90_ | TATA | ABE_NRN_ | spCas9 | CAGCCCC | 3001 | CAGCCCCC | 3307 | AAT |
| ABE_NRN_ | box | 20 nt_3- | NRN | CAGCCTT | | AGCCUUUU | | |
| 20 nt_3- | | 9_021 | ABE | TTATACA | | AUAC | | |
| 9_021_-_ | | | | AT | | | | |
| 31946060_ | | | | | | | | |
| spCas9 | | | | | | | | |
| GuideDesign90_ | TATA | CBE_NRN_ | spCas9 | CAGCCCC | 3002 | CAGCCCCC | 3308 | AAT |
| CBE_NRN_ | box | 20 nt_3- | NRN | CAGCCTT | | AGCCUUUU | | |
| 20 nt_3- | | 9_022 | CBE | TTATACA | | AUAC | | |
| 9_022_-_ | | | | AT | | | | |
| 31946060_ | | | | | | | | |
| spCas9 | | | | | | | | |
| GuideDesign90_ | TATA | MBE_NRN_ | spCas9 | CAGCCCC | 3003 | CAGCCCCC | 3309 | AAT |
| MBE_NRN_ | box | 20 nt_3- | NRN | CAGCCTT | | AGCCUUUU | | |
| 20 nt_3- | | 9_023 | MBE | TTATACA | | AUAC | | |
| 9_023_-_ | | | | AT | | | | |
| 31946060_ | | | | | | | | |
| spCas9 | | | | | | | | |
| GuideDesign90_ | TATA | ABE_NRTH_ | spCas9 | CAGCCCC | 3004 | CAGCCCCC | 3310 | AATC |
| ABE_NRT_ | box | 20 nt_3- | NRTH | CAGCCTT | | AGCCUUUU | | |
| H_20 nt_3- | | 9_025 | ABE | TTATACA | | AUAC | | |
| 9_025_-_ | | | | ATC | | | | |
| 31946059_ | | | | | | | | |
| spCas9 | | | | | | | | |
| GuideDesign90_ | TATA | CBE_NRTH_ | spCas9 | CAGCCCC | 3005 | CAGCCCCC | 3311 | AATC |
| CBE_NRTH_ | box | 20 nt_4- | NRTH | CAGCCTT | | AGCCUUUU | | |
| 20 nt_4- | | 9_026 | CBE | TTATACA | | AUAC | | |
| 9_026_-_ | | | | ATC | | | | |
| 31946059_ | | | | | | | | |
| spCas9 | | | | | | | | |
| GuideDesign90_ | TATA | CBE_ | saCas9 | CCACCAG | 3006 | CCACCAGC | 3312 | TACAA |
| CBE_NNNRRT_ | box | NNNRRT_ | KKH | CCCCCAG | | CCCCAGCC | | T |
| 21 nt_3- | | 21 nt_3- | CBE | CCTTTTA | | UUUUA | | |
| 12_015_-_ | | 12_015 | | TACAAT | | | | |
| 31946060_ | | | | | | | | |
| saCas9 | | | | | | | | |
| GuideDesign90_ | TATA | ABE_NRN_ | spCas9 | CCAGCCC | 3007 | CCAGCCCC | 3313 | CAA |
| ABE_NRN_ | box | 20 nt_3- | NRN | CCAGCCT | | CAGCCUUU | | |
| 20 nt_3- | | 9_021 | ABE | TTTATAC | | UAUA | | |
| 9_021_-_ | | | | AA | | | | |
| 31946061_ | | | | | | | | |
| spCas9 | | | | | | | | |
| GuideDesign90_ | TATA | CBE_NRN_ | spCas9 | CCAGCCC | 3008 | CCAGCCCC | 3314 | CAA |
| CBE_NRN_ | box | 20 nt_3- | NRN | CCAGCCT | | CAGCCUUU | | |
| 20 nt_3- | | 9_022 | CBE | TTTATAC | | UAUA | | |
| 9_022_-_ | | | | AA | | | | |
| 31946061_ | | | | | | | | |
| spCas9 | | | | | | | | |
| GuideDesign90_ | TATA | MBE_NRN_ | spCas9 | CCAGCCC | 3009 | CCAGCCCC | 3315 | CAA |
| MBE_NRN_ | box | 20 nt_3- | NRN | CCAGCCT | | CAGCCUUU | | |
| 20 nt_3- | | 9_023 | MBE | TTTATAC | | UAUA | | |
| 9_023_-_ | | | | AA | | | | |
| 31946061_ | | | | | | | | |
| spCas9 | | | | | | | | |
| GuideDesign90_ | TATA | CBE_NRRH_ | spCas9 | CCAGCCC | 3010 | CCAGCCCC | 3316 | CAAT |
| CBE_NRR_ | box | 20 nt_3- | NRRH | CCAGCCT | | CAGCCUUU | | |
| H_20 nt_3- | | 9_031 | CBE | TTTATAC | | UAUA | | |
| 9_031_- | | | | AAT | | | | |
| 31946060_ | | | | | | | | |
| spCas9 | | | | | | | | |
| GuideDesign90_ | TATA | ABE_NG_ | spCas9 | CCAGCCT | 3011 | CCAGCCUU | 3317 | TG |
| ABE_NG_20 nt_ | box | 20 nt_3- | NG | TTTATAC | | UUAUACAA | | |
| 3-9_027_-_ | | 9_027 | ABE | AATCTGT | | UCUG | | |
| 31946055_ | | | | G | | | | |
| spCas9 | | | | | | | | |
| GuideDesign90_ | TATA | CBE_NG_ | spCas9 | CCAGCCT | 3012 | CCAGCCUU | 3318 | TG |
| CBE_NG_20 nt_ | box | 20 nt_4- | NG | TTTATAC | | UUAUACAA | | |
| 4-9_028_-_ | | 9_028 | CBE | AATCTGT | | UCUG | | |
| 31946055_ | | | | G | | | | |
| spCas9 | | | | | | | | |
| GuideDesign90_ | TATA | ABE_NRN_ | spCas9 | CCAGCCT | 3013 | CCAGCCUU | 3319 | TGT |
| ABE_NRN_ | box | 20 nt_3- | NRN | TTTATAC | | UUAUACAA | | |
| 20 nt_3- | | 9_021 | ABE | AATCTGT | | UCUG | | |
| 9_021_- | | | | GT | | | | |
| 31946054_ | | | | | | | | |
| spCas9 | | | | | | | | |
| GuideDesign90_ | TATA | CBE_NRN_ | spCas9 | CCAGCCT | 3014 | CCAGCCUU | 3320 | TGT |
| CBE_NRN_ | box | 20 nt_3- | NRN | TTTATAC | | UUAUACAA | | |
| 20 nt_3- | | 9_022 | CBE | AATCTGT | | UCUG | | |
| 9_022_-_ | | | | GT | | | | |
| 31946054_ | | | | | | | | |
| spCas9 | | | | | | | | |
| GuideDesign90_ | TATA | MBE_NRN_ | spCas9 | CCAGCCT | 3015 | CCAGCCUU | 3321 | TGT |
| MBE_NRN_ | box | 20 nt_3- | NRN | TTTATAC | | UUAUACAA | | |
| 20 nt_3- | | 9_023 | MBE | AATCTGT | | UCUG | | |
| 9_023_- | | | | GT | | | | |
| 31946054_ | | | | | | | | |
| spCas9 | | | | | | | | |
| GuideDesign90_ | TATA | ABE_NRTH_ | spCas9 | CCAGCCT | 3016 | CCAGCCUU | 3322 | TGTT |
| ABE_NRTH_ | box | 20 nt_3- | NRTH | TTTATAC | | UUAUACAA | | |
| 20 nt_3- | | 9_025 | ABE | AATCTGT | | UCUG | | |
| 9_025_-_ | | | | GTT | | | | |
| 31946053_ | | | | | | | | |
| spCas9 | | | | | | | | |
| GuideDesign90_ | TATA | CBE_NRTH_ | spCas9 | CCAGCCT | 3017 | CCAGCCUU | 3323 | TGTT |
| CBE_NRTH_ | box | 20 nt_4- | NRTH | TTTATAC | | UUAUACAA | | |
| 20 nt_4- | | 9_026 | CBE | AATCTGT | | UCUG | | |
| 9_026_-_ | | | | GTT | | | | |
| 31946053_ | | | | | | | | |
| spCas9 | | | | | | | | |
| GuideDesign90_ | TATA | ABE_NG_ | spCas9 | CCCCAGC | 3018 | CCCCAGCC | 3324 | TG |
| ABE_NG_20 nt_ | box | 20 nt_3- | NG | CTTTTAT | | UUUUAUAC | | |
| 3-9_027_-_ | | 9_027 | ABE | ACAATCT | | AAUC | | |
| 31946057_ | | | | G | | | | |
| spCas9 | | | | | | | | |
| GuideDesign90_ | TATA | CBE_NG_ | spCas9 | CCCCAGC | 3019 | CCCCAGCC | 3325 | TG |
| CBE_NG_20 nt_ | box | 20 nt_4- | NG | CTTTTAT | | UUUUAUAC | | |
| 4-9_028_-_ | | 9_028 | CBE | ACAATCT | | AAUC | | |
| 31946057_ | | | | G | | | | |
| spCas9 | | | | | | | | |
| GuideDesign90_ | TATA | ABE_NRN_ | spCas9 | CCCCAGC | 3020 | CCCCAGCC | 3326 | TGT |
| ABE_NRN_ | box | 20 nt_3- | NRN | CTTTTAT | | UUUUAUAC | | |
| 20 nt_3- | | 9_021 | ABE | ACAATCT | | AAUC | | |
| 9_021_-_ | | | | GT | | | | |
| 31946056_ | | | | | | | | |
| spCas9 | | | | | | | | |
| GuideDesign90_ | TATA | CBE_NRN_ | spCas9 | CCCCAGC | 3021 | CCCCAGCC | 3327 | TGT |
| CBE_NRN_ | box | 20 nt_3- | NRN | CTTTTAT | | UUUUAUAC | | |
| 20 nt_3- | | 9_022 | CBE | ACAATCT | | AAUC | | |
| 9_022_-_ | | | | GT | | | | |
| 31946056_ | | | | | | | | |
| spCas9 | | | | | | | | |
| GuideDesign90_ | TATA | MBE_NRN_ | spCas9 | CCCCAGC | 3022 | CCCCAGCC | 3328 | TGT |
| MBE_NRN_ | box | 20 nt_3- | NRN | CTTTTAT | | UUUUAUAC | | |
| 20 nt_3- | | 9_023 | MBE | ACAATCT | | AAUC | | |
| 9_023_-_ | | | | GT | | | | |
| 31946056_ | | | | | | | | |
| spCas9 | | | | | | | | |
| GuideDesign90_ | TATA | ABE_NGG_ | spCas9 | CTGGGGG | 3023 | CUGGGGGC | 3329 | GGG |
| ABE_NGG_ | box | 20 nt_3- | ABE | CTGGTGG | | UGGUGGGG | | |
| 20 nt_3- | | 9_002 | | GGAGCAG | | AGCA | | |
| 9_002_+_ | | | | GG | | | | |
| 31946073_spCas9 | | | | | | | | |
| GuideDesign90_ | TATA | ABE_NGG_ | spCas9 | CTGGGGG | 3024 | CUGGGGGC | 3330 | GGG |
| ABE_NGG_ | box | 20 nt_3- | IBE | CTGGTGG | | UGGUGGGG | | |
| 20 nt_3- | | 12_018 | | GGAGCAG | | AGCA | | |
| 12_018_+_ | | | | GG | | | | |
| 31946073_spCas9 | | | | | | | | |
| GuideDesign90_ | TATA | CBE_NGG_ | spCas9 | CTGGGGG | 3025 | CUGGGGGC | 3331 | GGG |
| CBE_NGG_ | box | 20 nt_4- | CBE | CTGGTGG | | UGGUGGGG | | |
| 20 nt_4- | | 9_003 | | GGAGCAG | | AGCA | | |
| 9_003_+_ | | | | GG | | | | |
| 31946073_spCas9 | | | | | | | | |
| GuideDesign90_ | TATA | CBE_NGG_ | spCas9 | CTGGGGG | 3026 | CUGGGGGC | 3332 | GGG |
| CBE_NGG_ | box | 20 nt_3- | NGG | CTGGTGG | | UGGUGGGG | | |
| 20 nt_3- | | 9_029 | CBE | GGAGCAG | | AGCA | | |
| 9_029_+_ | | | | GG | | | | |
| 31946073_spCas9 | | | | | | | | |
| GuideDesign90_ | TATA | CBE_NGG_ | SpCas9 | CTGGGGG | 3027 | CUGGGGGC | 3333 | GGG |
| CBE_NGG_ | box | 20 nt_3- | IBE | CTGGTGG | | UGGUGGGG | | |
| 20 nt_3- | | 16_033 | CBE | GGAGCAG | | AGCA | | |
| 16_033_+_ | | | | GG | | | | |
| 31946073_SpCas9 | | | | | | | | |
| GuideDesign90_ | TATA | ABE_NG_ | spCas9 | CTGGGGG | 3028 | CUGGGGGC | 3334 | GG |
| ABE_NG_ | box | 20 nt_3- | NG | CTGGTGG | | UGGUGGGG | | |
| 20 nt_3- | | 9_027 | ABE | GGAGCAG | | AGCA | | |
| 9_027_+_ | | | | G | | | | |
| 31946073_spCas9 | | | | | | | | |
| GuideDesign90_ | TATA | CBE_NG_ | spCas9 | CTGGGGG | 3029 | CUGGGGGC | 3335 | GG |
| CBE_NG_ | box | 20 nt_4- | NG | CTGGTGG | | UGGUGGGG | | |
| 20 nt_4- | | 9_028 | CBE | GGAGCAG | | AGCA | | |
| 9_028_+_ | | | | G | | | | |
| 31946073_spCas9 | | | | | | | | |
| GuideDesign90_ | TATA | ABE_NRN_ | spCas9 | CTGGGGG | 3030 | CUGGGGGC | 3336 | GGG |
| ABE_NRN_ | box | 20 nt_3- | NRN | CTGGTGG | | UGGUGGGG | | |
| 20 nt_3- | | 9_021 | ABE | GGAGCAG | | AGCA | | |
| 9_021_+_ | | | | GG | | | | |
| 31946073_spCas9 | | | | | | | | |
| GuideDesign90_ | TATA | CBE_NRN_ | spCas9 | CTGGGGG | 3031 | CUGGGGGC | 3337 | GGG |
| CBE_NRN_ | box | 20 nt_3- | NRN | CTGGTGG | | UGGUGGGG | | |
| 20 nt_3- | | 9_022 | CBE | GGAGCAG | | AGCA | | |
| 9_022_+_ | | | | GG | | | | |
| 31946073_spCas9 | | | | | | | | |
| GuideDesign90_ | TATA | MBE_NRN_ | spCas9 | CTGGGGG | 3032 | CUGGGGGC | 3338 | GGG |
| MBE_NRN_ | box | 20 nt_3- | NRN | CTGGTGG | | UGGUGGGG | | |
| 20 nt_3- | | 9_023 | MBE | GGAGCAG | | AGCA | | |
| 9_023_+_ | | | | GG | | | | |
| 31946073_spCas9 | | | | | | | | |
| GuideDesign90_ | TATA | ABE_NGG_ | spCas9 | CTTTTAT | 3033 | CUUUUAUA | 3339 | TGG |
| ABE_NGG_ | box | 20 nt_3- | IBE | ACAATCT | | CAAUCUGU | | |
| 20 nt_3- | | 12_018 | | GTGTTCT | | GUUC | | |
| 12_018_-_ | | | | GG | | | | |
| 31946049_ | | | | | | | | |
| spCas9 | | | | | | | | |
| GuideDesign90_ | TATA | ABE_NGG_ | SpCas9 | CTTTTAT | 3034 | CUUUUAUA | 3340 | TGG |
| ABE_NGG_ | box | 20 nt_13- | IBE ex | ACAATCT | | CAAUCUGU | | |
| 20 nt_13- | | 16_032 | panded | GTGTTCT | | GUUC | | |
| 16_032_-_ | | | ABE | GG | | | | |
| 31946049_ | | | | | | | | |
| SpCas9 | | | | | | | | |
| GuideDesign90_ | TATA | CBE_NGG_ | spCas9 | CTTTTAT | 3035 | CUUUUAUA | 3341 | TGG |
| CBE_NGG_ | box | 20 nt_4- | CBE | ACAATCT | | CAAUCUGU | | |
| 20 nt_4- | | 9_003 | | GTGTTCT | | GUUC | | |
| 9_003_-_ | | | | GG | | | | |
| 31946049_ | | | | | | | | |
| spCas9 | | | | | | | | |
| GuideDesign90_ | TATA | CBE_NGG_ | spCas9 | CTTTTAT | 3036 | CUUUUAUA | 3342 | TGG |
| CBE_NGG_ | box | 20 nt_3- | NGG | ACAATCT | | CAAUCUGU | | |
| 20 nt_3- | | 9_029 | CBE | GTGTTCT | | GUUC | | |
| 9_029_-_ | | | | GG | | | | |
| 31946049_ | | | | | | | | |
| spCas9 | | | | | | | | |
| GuideDesign90_ | TATA | CBE_NGG_ | SpCas9 | CTTTTAT | 3037 | CUUUUAUA | 3343 | TGG |
| CBE_NGG_ | box | 20 nt_3- | IBE | ACAATCT | | CAAUCUGU | | |
| 20 nt_3- | | 16_033 | CBE | GTGTTCT | | GUUC | | |
| 16_033_-_ | | | | GG | | | | |
| 31946049_ | | | | | | | | |
| SpCas9 | | | | | | | | |
| GuideDesign90_ | TATA | ABE_NG_ | spCas9 | CTTTTAT | 3038 | CUUUUAUA | 3344 | TG |
| ABE_NG_20 nt_ | box | 20 nt_3- | NG | ACAATCT | | CAAUCUGU | | |
| 3-9_027_-_ | | 9_027 | ABE | GTGTTCT | | GUUC | | |
| 31946050_ | | | | G | | | | |
| spCas9 | | | | | | | | |
| GuideDesign90_ | TATA | CBE_NG_ | spCas9 | CTTTTAT | 3039 | CUUUUAUA | 3345 | TG |
| CBE_NG_20 nt_ | box | 20 nt_4- | NG | ACAATCT | | CAAUCUGU | | |
| 4-9_028_-_ | | 9_028 | CBE | GTGTTCT | | GUUC | | |
| 31946050_ | | | | G | | | | |
| spCas9 | | | | | | | | |
| GuideDesign90_ | TATA | ABE_NRN_ | spCas9 | CTTTTAT | 3040 | CUUUUAUA | 3346 | TGG |
| ABE_NRN_ | box | 20 nt_3- | NRN | ACAATCT | | CAAUCUGU | | |
| 20 nt_3- | | 9_021 | ABE | GTGTTCT | | GUUC | | |
| 9_021_-_ | | | | GG | | | | |
| 31946049_ | | | | | | | | |
| spCas9 | | | | | | | | |
| GuideDesign90_ | TATA | CBE_NRN_ | spCas9 | CTTTTAT | 3041 | CUUUUAUA | 3347 | TGG |
| CBE_NRN_ | box | 20 nt_3- | NRN | ACAATCT | | CAAUCUGU | | |
| 20 nt_3- | | 9_022 | CBE | GTGTTCT | | GUUC | | |
| 9_022_-_ | | | | GG | | | | |
| 31946049_ | | | | | | | | |
| spCas9 | | | | | | | | |
| GuideDesign90_ | TATA | MBE_NRN_ | spCas9 | CTTTTAT | 3042 | CUUUUAUA | 3348 | TGG |
| MBE_NRN_ | box | 20 nt_3- | NRN | ACAATCT | | CAAUCUGU | | |
| 20 nt_3- | | 9_023 | MBE | GTGTTCT | | GUUC | | |
| 9_023_-_ | | | | GG | | | | |
| 31946049_ | | | | | | | | |
| spCas9 | | | | | | | | |
| GuideDesign90_ | TATA | CBE_NRRH_ | spCas9 | CTTTTAT | 3043 | CUUUUAUA | 3349 | TGGC |
| CBE_NRRH_ | box | 20 nt_3- | NRRH | ACAATCT | | CAAUCUGU | | |
| 20 nt_3- | | 9_031 | CBE | GTGTTCT | | GUUC | | |
| 9_031_-_ | | | | GGC | | | | |
| 31946048_ | | | | | | | | |
| spCas9 | | | | | | | | |
| GuideDesign90_ | TATA | ABE_NGG_ | spCas9 | GCTGGGG | 3044 | GCUGGGGG | 3350 | AGG |
| ABE_NGG | box | 20 nt_3- | ABE | GCTGGTG | | CUGGUGGG | | |
| 20 nt_3- | | 9_002 | | GGGAGCA | | GAGC | | |
| 9_002_+_ | | | | GG | | | | |
| 31946072_spCas9 | | | | | | | | |
| GuideDesign90_ | TATA | ABE_NGG_ | spCas9 | GCTGGGG | 3045 | GCUGGGGG | 3351 | AGG |
| ABE_NGG_ | box | 20 nt_3- | IBE | GCTGGTG | | CUGGUGGG | | |
| 20 nt_3- | | 12_018 | | GGGAGCA | | GAGC | | |
| 12_018_+_ | | | | GG | | | | |
| 31946072_spCas9 | | | | | | | | |
| GuideDesign90_ | TATA | CBE_NGG_ | spCas9 | GCTGGGG | 3046 | GCUGGGGG | 3352 | AGG |
| CBE_NGG_ | box | 20 nt_4- | CBE | GCTGGTG | | CUGGUGGG | | |
| 20 nt_4- | | 9_003 | | GGGAGCA | | GAGC | | |
| 9_003_+_ | | | | GG | | | | |
| 31946072_spCas9 | | | | | | | | |
| GuideDesign90_ | TATA | CBE_NGG_ | spCas9 | GCTGGGG | 3047 | GCUGGGGG | 3353 | AGG |
| CBE_NGG_ | box | 20 nt_3- | NGG | GCTGGTG | | CUGGUGGG | | |
| 20 nt_3- | | 9_029 | CBE | GGGAGCA | | GAGC | | |
| 9_029_+_ | | | | GG | | | | |
| 31946072_spCas9 | | | | | | | | |
| GuideDesign90_ | TATA | CBE_NGG_ | SpCas9 | GCTGGGG | 3048 | GCUGGGGG | 3354 | AGG |
| CBE_NGG_ | box | 20 nt_3- | IBE | GCTGGTG | | CUGGUGGG | | |
| 20 nt_3- | | 16_033 | CBE | GGGAGCA | | GAGC | | |
| 16_033_+_ | | | | GG | | | | |
| 31946072_SpCas9 | | | | | | | | |
| GuideDesign90_ | TATA | ABE_NG_ | spCas9 | GCTGGGG | 3049 | GCUGGGGG | 3355 | AG |
| ABE_NG_ | box | 20 nt_3- | NG | GCTGGTG | | CUGGUGGG | | |
| 20 nt_3- | | 9_027 | ABE | GGGAGCA | | GAGC | | |
| 9_027_+_ | | | | G | | | | |
| 31946072_spCas9 | | | | | | | | |
| GuideDesign90_ | TATA | CBE_NG_ | spCas9 | GCTGGGG | 3050 | GCUGGGGG | 3356 | AG |
| CBE_NG_ | box | 20 nt_4- | NG | GCTGGTG | | | | |
| 20 nt_4- | | 9_028 | CBE | GGGAGCA | | CUGGUGGG | | |
| 9_028_+_ | | | | G | | GAGC | | |
| 31946072_spCas9 | | | | | | | | |
| GuideDesign90_ | TATA | ABE_NRN_ | spCas9 | GCTGGGG | 3051 | GCUGGGGG | 3357 | AGG |
| ABE_NRN_ | box | 20 nt_3- | NRN | GCTGGTG | | CUGGUGGG | | |
| 20 nt_3- | | 9_021 | ABE | GGGAGCA | | GAGC | | |
| 9_021_+_ | | | | GG | | | | |
| 31946072_spCas9 | | | | | | | | |
| GuideDesign90_ | TATA | CBE_NRN_ | spCas9 | GCTGGGG | 3052 | GCUGGGGG | 3358 | AGG |
| CBE_NRN_ | box | 20 nt_3- | NRN | GCTGGTG | | CUGGUGGG | | |
| 20 nt_3- | | 9_022 | CBE | GGGAGCA | | GAGC | | |
| 9_022_+_ | | | | GG | | | | |
| 31946072_spCas9 | | | | | | | | |
| GuideDesign90_ | TATA | MBE_NRN_ | spCas9 | GCTGGGG | 3053 | GCUGGGGG | 3359 | AGG |
| MBE_NRN_ | box | 20 nt_3- | NRN | GCTGGTG | | CUGGUGGG | | |
| 20 nt_3- | | 9_023 | MBE | GGGAGCA | | GAGC | | |
| 9_023_+_ | | | | GG | | | | |
| 31946072_spCas9 | | | | | | | | |
| GuideDesign90_ | TATA | ABE_NRN_ | spCas9 | GGCTGGG | 3054 | GGCUGGGG | 3360 | CAG |
| ABE_NRN_ | box | 20 nt_3- | NRN | GGCTGGT | | GCUGGUGG | | |
| 20 nt_3- | | 9_021 | ABE | GGGGAGC | | GGAG | | |
| 9_021_+_ | | | | AG | | | | |
| 31946071_spCas9 | | | | | | | | |
| GuideDesign90_ | TATA | CBE_NRN_ | spCas9 | GGCTGGG | 3055 | GGCUGGGG | 3361 | CAG |
| CBE_NRN_ | box | 20 nt_3- | NRN | GGCTGGT | | GCUGGUGG | | |
| 20 nt_3- | | 9_022 | CBE | GGGGAGC | | GGAG | | |
| 9_022_+_ | | | | AG | | | | |
| 31946071_spCas9 | | | | | | | | |
| GuideDesign90_ | TATA | MBE_NRN_ | spCas9 | GGCTGGG | 3056 | GGCUGGGG | 3362 | CAG |
| MBE_NRN_ | box | 20 nt_3- | NRN | GGCTGGT | | GCUGGUGG | | |
| 20 nt_3- | | 9_023 | MBE | GGGGAGC | | GGAG | | |
| 9_023_+_ | | | | AG | | | | |
| 31946071_spCas9 | | | | | | | | |
| GuideDesign90_ | TATA | ABE_NGG_ | spCas9 | TAAAAGG | 3057 | UAAAAGGC | 3363 | GGG |
| ABE_NGG_ | box | 20 nt_3- | IBE | CTGGGGG | | UGGGGGCU | | |
| 20 nt_3- | | 12_018 | | CTGGTGG | | GGUG | | |
| 12_018_+_ | | | | GG | | | | |
| 31946066_spCas9 | | | | | | | | |
| GuideDesign90_ | TATA | CBE_NGG_ | spCas9 | TAAAAGG | 3058 | UAAAAGGC | 3364 | GGG |
| CBE_NGG_ | box | 20 nt_4- | CBE | CTGGGGG | | UGGGGGCU | | |
| 20 nt_4- | | 9_003 | | CTGGTGG | | GGUG | | |
| 9_003_+_ | | | | GG | | | | |
| 31946066_spCas9 | | | | | | | | |
| GuideDesign90_ | TATA | CBE_NGG_ | spCas9 | TAAAAGG | 3059 | UAAAAGGC | 3365 | GGG |
| CBE_NGG_ | box | 20 nt_3- | NGG | CTGGGGG | | UGGGGGCU | | |
| 20 nt_3- | | 9_029 | CBE | CTGGTGG | | GGUG | | |
| 9_029_+_ | | | | GG | | | | |
| 31946066_spCas9 | | | | | | | | |
| GuideDesign90_ | TATA | CBE_NGG_ | SpCas9 | TAAAAGG | 3060 | UAAAAGGC | 3366 | GGG |
| CBE_NGG_ | box | 20 nt_3- | IBE | CTGGGGG | | UGGGGGCU | | |
| 20 nt_3- | | 16_033 | CBE | CTGGTGG | | GGUG | | |
| 16_033_+_ | | | | GG | | | | |
| 31946066_SpCas9 | | | | | | | | |
| GuideDesign90_ | TATA | ABE_NG_ | spCas9 | TAAAAGG | 3061 | UAAAAGGC | 3367 | GG |
| ABE_NG_ | box | 20 nt_3- | NG | CTGGGGG | | UGGGGGCU | | |
| 20 nt_3- | | 9_027 | ABE | CTGGTGG | | GGUG | | |
| 9_027_+_ | | | | G | | | | |
| 31946066_spCas9 | | | | | | | | |
| GuideDesign90_ | TATA | CBE_NG_ | spCas9 | TAAAAGG | 3062 | UAAAAGGC | 3368 | GG |
| CBE_NG_ | box | 20 nt_4- | NG | CTGGGGG | | UGGGGGCU | | |
| 20 nt_4- | | 9_028 | CBE | CTGGTGG | | GGUG | | |
| 9_028_+_ | | | | G | | | | |
| 31946066_spCas9 | | | | | | | | |
| GuideDesign90_ | TATA | ABE_NRN_ | spCas9 | TAAAAGG | 3063 | UAAAAGGC | 3369 | GGG |
| ABE_NRN_ | box | 20 nt_3- | NRN | CTGGGGG | | UGGGGGCU | | |
| 20 nt_3- | | 9_021 | ABE | | | GGUG | | |
| 9_021_+_ | | | | CTGGTGG | | | | |
| 31946066_spCas9 | | | | GG | | | | |
| GuideDesign90_ | TATA | CBE_NRN_ | spCas9 | TAAAAGG | 3064 | UAAAAGGC | 3370 | GGG |
| CBE_NRN_ | box | 20 nt_3- | NRN | CTGGGGG | | UGGGGGCU | | |
| 20 nt_3- | | 9_022 | CBE | CTGGTGG | | GGUG | | |
| 9_022_+_ | | | | GG | | | | |
| 31946066_spCas9 | | | | | | | | |
| GuideDesign90_ | TATA | MBE_NRN_ | spCas9 | TAAAAGG | 3065 | UAAAAGGC | 3371 | GGG |
| MBE_NRN_ | box | 20 nt_3- | NRN | CTGGGGG | | UGGGGGCU | | |
| 20 nt_3- | | 9_023 | MBE | CTGGTGG | | GGUG | | |
| 9_023_+_ | | | | GG | | | | |
| 31946066_spCas9 | | | | | | | | |
| GuideDesign90_ | TATA | CBE_NRRH_ | spCas9 | TAAAAGG | 3066 | UAAAAGGC | 3372 | GGGA |
| CBE_NRR_ | box | 20 nt_3- | NRRH | CTGGGGG | | UGGGGGCU | | |
| H_20 nt_3- | | 9_031 | CBE | CTGGTGG | | GGUG | | |
| 9_031_+_ | | | | GGA | | | | |
| 31946066_spCas9 | | | | | | | | |
| GuideDesign90_ | TATA | ABE_NGG_ | spCas9 | TATAAAA | 3067 | UAUAAAAG | 3373 | TGG |
| ABE_NGG_ | box | 20 nt_3- | IBE | GGCTGGG | | GCUGGGGG | | |
| 20 nt_3- | | 12_018 | | GGCTGGT | | CUGG | | |
| 12_018_+_ | | | | GG | | | | |
| 31946064_spCas9 | | | | | | | | |
| GuideDesign90_ | TATA | ABE_NGG_ | SpCas9 | TATAAAA | 3068 | UAUAAAAG | 3374 | TGG |
| ABE_NGG_ | box | 20 nt_13- | IBE_ | GGCTGGG | | GCUGGGGG | | |
| 20 nt_13- | | 16_032 | expanded | GGCTGGT | | CUGG | | |
| 16_032_+_ | | | ABE | GG | | | | |
| 31946064_SpCas9 | | | | | | | | |
| GuideDesign90_ | TATA | CBE_NGG_ | spCas9 | TATAAAA | 3069 | UAUAAAAG | 3375 | TGG |
| CBE_NGG_ | box | 20 nt_4- | CBE | GGCTGGG | | GCUGGGGG | | |
| 20 nt_4- | | 9_003 | | GGCTGGT | | CUGG | | |
| 9_003_+_ | | | | GG | | | | |
| 31946064_spCas9 | | | | | | | | |
| GuideDesign90_ | TATA | CBE_NGG_ | spCas9 | TATAAAA | 3070 | UAUAAAAG | 3376 | TGG |
| CBE_NGG_ | box | 20 nt_3- | NGG | GGCTGGG | | GCUGGGGG | | |
| 20 nt_3- | | 9_029 | CBE | GGCTGGT | | CUGG | | |
| 9_029_+_ | | | | GG | | | | |
| 31946064_spCas9 | | | | | | | | |
| GuideDesign90_ | TATA | CBE_NGG_ | SpCas9 | TATAAAA | 3071 | UAUAAAAG | 3377 | TGG |
| CBE_NGG_ | box | 20 nt_3- | IBE | GGCTGGG | | GCUGGGGG | | |
| 20 nt_3- | | 16_033 | CBE | GGCTGGT | | CUGG | | |
| 16_033_+_ | | | | GG | | | | |
| 31946064_SpCas9 | | | | | | | | |
| Guide_Design90_ | TATA | ABE_NG_ | spCas9 | TATAAAA | 3072 | UAUAAAAG | 3378 | TG |
| ABE_NG_ | box | 20 nt_3- | NG | GGCTGGG | | GCUGGGGG | | |
| 20 nt_3- | | 9_027 | ABE | GGCTGGT | | CUGG | | |
| 9_027_+_ | | | | G | | | | |
| 31946064_spCas9 | | | | | | | | |
| GuideDesign90_ | TATA | CBE_NG_ | spCas9 | TATAAAA | 3073 | UAUAAAAG | 3379 | TG |
| CBE_NG_ | box | 20 nt_4- | NG | GGCTGGG | | GCUGGGGG | | |
| 20 nt_4- | | 9_028 | CBE | | | CUGG | | |
| 9_028_+_ | | | | GGCTGGT | | | | |
| 31946064_spCas9 | | | | G | | | | |
| GuideDesign90_ | TATA | ABE_NRN_ | spCas9 | TATAAAA | 3074 | UAUAAAAG | 3380 | TGG |
| ABE_NRN_ | box | 20 nt_3- | NRN | GGCTGGG | | GCUGGGGG | | |
| 20 nt_3- | | 9_021 | ABE | GGCTGGT | | CUGG | | |
| 9_021_+_ | | | | GG | | | | |
| 31946064_spCas9 | | | | | | | | |
| GuideDesign90_ | TATA | CBE_NRN_ | spCas9 | TATAAAA | 3075 | UAUAAAAG | 3381 | TGG |
| CBE_NRN_ | box | 20 nt_3- | NRN | GGCTGGG | | GCUGGGGG | | |
| 20 nt_3- | | 9_022 | CBE | GGCTGGT | | CUGG | | |
| 9_022_+_ | | | | GG | | | | |
| 31946064_spCas9 | | | | | | | | |
| GuideDesign90_ | TATA | MBE_NRN_ | spCas9 | TATAAAA | 3076 | UAUAAAAG | 3382 | TGG |
| MBE_NRN_ | box | 20 nt_3- | NRN | GGCTGGG | | GCUGGGGG | | |
| 20 nt_3- | | 9_023 | MBE | GGCTGGT | | CUGG | | |
| 9_023_+_ | | | | GG | | | | |
| 31946064_spCas9 | | | | | | | | |
| GuideDesign90_ | TATA | ABE_VTTN_ | cas12b | ATTGTAT | 3077 | UAUAAAAG | 3383 | ATTG |
| ABE_VTTN_ | box | 22 nt_5- | ABE | AAAAGGC | | GCUGGGGG | | |
| 22 nt_5- | | 9_017 | | TGGGGGC | | CUGGUG | | |
| 9_017_+_ | | | | TGGTG | | | | |
| 31946060_cas12b | | | | | | | | |
| GuideDesign90_ | TATA | ABE_VTTN_ | cas12b | CTTTTAT | 3078 | UAUACAAU | 3384 | CTTT |
| ABE_VTTN_ | box | 22 nt_5- | ABE | ACAATCT | | CUGUGUUC | | |
| 22 nt_5- | | 9_017 | | GTGTTCT | | UGGCAC | | |
| 9_017_-_ | | | | GGCAC | | | | |
| 31946046_ | | | | | | | | |
| cas12b | | | | | | | | |
| GuideDesign90_ | TATA | ABE_NGG_ | spCas9 | TGGGGGC | 3079 | UGGGGGCU | 3385 | GGG |
| ABE_NGG_ | box | 20 nt_3- | ABE | TGGTGGG | | GGUGGGGA | | |
| 20 nt_3- | | 9_002 | | GAGCAGG | | GCAG | | |
| 9_002_+_ | | | | GG | | | | |
| 31946074_spCas9 | | | | | | | | |
| GuideDesign90_ | TATA | ABE_NGG_ | spCas9 | TGGGGGC | 3080 | UGGGGGCU | 3386 | GGG |
| ABE_NGG_ | box | 20 nt_3- | IBE | TGGTGGG | | GGUGGGGA | | |
| 20 nt_3- | | 12_018 | | GAGCAGG | | GCAG | | |
| 12_018_+_ | | | | GG | | | | |
| 31946074_spCas9 | | | | | | | | |
| GuideDesign90_ | TATA | CBE_NGG_ | spCas9 | TGGGGGC | 3081 | UGGGGGCU | 3387 | GGG |
| CBE_NGG_ | box | 20 nt_3- | NGG | TGGTGGG | | GGUGGGGA | | |
| 20 nt_3- | | 9_029 | CBE | GAGCAGG | | GCAG | | |
| 9_029_+_ | | | | GG | | | | |
| 31946074_spCas9 | | | | | | | | |
| GuideDesign90_ | TATA | CBE_NGG_ | SpCas9 | TGGGGGC | 3082 | UGGGGGCU | 3388 | GGG |
| CBE_NGG_ | box | 20 nt_3- | IBE | TGGTGGG | | GGUGGGGA | | |
| 20 nt_3- | | 16_033 | CBE | GAGCAGG | | GCAG | | |
| 16_033_+_ | | | | GG | | | | |
| 31946074_SpCas9 | | | | | | | | |
| GuideDesign90_ | TATA | ABE_NG_ | spCas9 | TGGGGGC | 3083 | UGGGGGCU | 3389 | GG |
| ABE_NG_ | box | 20 nt_3- | NG | TGGTGGG | | GGUGGGGA | | |
| 20 nt_3- | | 9_027 | ABE | GAGCAGG | | GCAG | | |
| 9_027_+_ | | | | G | | | | |
| 31946074_spCas9 | | | | | | | | |
| GuideDesign90_ | TATA | ABE_NRN_ | spCas9 | TGGGGGC | 3084 | UGGGGGCU | 3390 | GGG |
| ABE_NRN_ | box | 20 nt_3- | NRN | TGGTGGG | | GGUGGGGA | | |
| 20 nt_3- | | 9_021 | ABE | GAGCAGG | | GCAG | | |
| 9_021_+_ | | | | GG | | | | |
| 31946074_spCas9 | | | | | | | | |
| GuideDesign90_ | TATA | CBE_NRN_ | spCas9 | TGGGGGC | 3085 | UGGGGGCU | 3391 | GGG |
| CBE_NRN_ | box | 20 nt_3- | NRN | TGGTGGG | | GGUGGGGA | | |
| 20 nt_3- | | 9_022 | CBE | GAGCAGG | | GCAG | | |
| 9_022_+_ | | | | GG | | | | |
| 31946074_spCas9 | | | | | | | | |
| GuideDesign90_ | TATA | MBE_NRN_ | spCas9 | TGGGGGC | 3086 | UGGGGGCU | 3392 | GGG |
| MBE_NRN_ | box | 20 nt_3- | NRN | TGGTGGG | | GGUGGGGA | | |
| 20 nt_3- | | 9_023 | MBE | GAGCAGG | | GCAG | | |
| 9_023_+_ | | | | GG | | | | |
| 31946074_spCas9 | | | | | | | | |
| GuideDesign90_ | TATA | ABE_NG_ | spCas9 | TGTATAA | 3087 | UGUAUAAA | 3393 | GG |
| ABE_NG_ | box | 20 nt_3- | NG | AAGGCTG | | AGGCUGGG | | |
| 20 nt_3- | | 9_027 | ABE | GGGGCTG | | GGCU | | |
| 9_027_+_ | | | | G | | | | |
| 31946062_spCas9 | | | | | | | | |
| GuideDesign90_ | TATA | CBE_NG_ | spCas9 | TGTATAA | 3088 | UGUAUAAA | 3394 | GG |
| CBE_NG_ | box | 20 nt_4- | NG | AAGGCTG | | AGGCUGGG | | |
| 20 nt_4- | | 9_028 | CBE | GGGGCTG | | GGCU | | |
| 9_028_+_ | | | | G | | | | |
| 31946062_spCas9 | | | | | | | | |
| GuideDesign90_ | TATA | ABE_NRN_ | spCas9 | TGTATAA | 3089 | UGUAUAAA | 3395 | GGT |
| ABE_NRN_ | box | 20 nt_3- | NRN | AAGGCTG | | AGGCUGGG | | |
| 20 nt_3- | | 9_021 | ABE | GGGGCTG | | GGCU | | |
| 9_021_+_ | | | | GT | | | | |
| 31946062_spCas9 | | | | | | | | |
| GuideDesign90_ | TATA | CBE_NRN_ | spCas9 | TGTATAA | 3090 | UGUAUAAA | 3396 | GGT |
| CBE_NRN_ | box | 20 nt_3- | NRN | AAGGCTG | | AGGCUGGG | | |
| 20 nt_3- | | 9_022 | CBE | GGGGCTG | | GGCU | | |
| 9_022_+_ | | | | GT | | | | |
| 31946062_spCas9 | | | | | | | | |
| GuideDesign90_ | TATA | MBE_NRN_ | spCas9 | TGTATAA | 3091 | UGUAUAAA | 3397 | GGT |
| MBE_NRN_ | box | 20 nt_3- | NRN | AAGGCTG | | AGGCUGGG | | |
| 20 nt_3- | | 9_023 | MBE | GGGGCTG | | GGCU | | |
| 9_023_+_ | | | | GT | | | | |
| 31946062_spCas9 | | | | | | | | |
| GuideDesign90_ | TATA | ABE_NRN_ | spCas9 | TTATACA | 3092 | UUAUACAA | 3398 | CAC |
| ABE_NRN_ | box | 20 nt_3- | NRN | ATCTGTG | | UCUGUGUU | | |
| 20 nt_3- | | 9_021 | ABE | TTCTGGC | | CUGG | | |
| 9_021_-_ | | | | AC | | | | |
| 31946046_ | | | | | | | | |
| spCas9 | | | | | | | | |
| GuideDesign90_ | TATA | CBE_NRN_ | spCas9 | TTATACA | 3093 | UUAUACAA | 3399 | CAC |
| CBE_NRN_ | box | 20 nt_3- | NRN | ATCTGTG | | UCUGUGUU | | |
| 20 nt_3- | | 9_022 | CBE | TTCTGGC | | CUGG | | |
| 9_022_-_ | | | | AC | | | | |
| 31946046_ | | | | | | | | |
| spCas9 | | | | | | | | |
| GuideDesign90_ | TATA | MBE_NRN_ | spCas9 | TTATACA | 3094 | UUAUACAA | 3400 | CAC |
| MBE_NRN_ | box | 20 nt_3- | NRN | ATCTGTG | | UCUGUGUU | | |
| 20 nt_3- | | 9_023 | MBE | TTCTGGC | | CUGG | | |
| 9_023_-_ | | | | AC | | | | |
| 31946046_ | | | | | | | | |
| spCas9 | | | | | | | | |
| GuideDesign90_ | TATA | ABE_NRCH_ | spCas9 | TTATACA | 3095 | UUAUACAA | 3401 | CACC |
| ABE_NRC_ | box | 20 nt_3- | NRCH | ATCTGTG | | UCUGUGUU | | |
| H_20 nt_3- | | 9_024 | ABE | TTCTGGC | | CUGG | | |
| 9_024_-_ | | | | ACC | | | | |
| 31946045_ | | | | | | | | |
| spCas9 | | | | | | | | |
| GuideDesign90_ | TATA | CBE_NRCH_ | spCas9 | TTATACA | 3096 | UUAUACAA | 3402 | CACC |
| CBE_NRCH_ | box | 20 nt_3- | NRCH | ATCTGTG | | UCUGUGUU | | |
| 20 nt_3- | | 9_030 | CBE | TTCTGGC | | CUGG | | |
| 9_030_-_ | | | | ACC | | | | |
| 31946045_ | | | | | | | | |
| spCas9 | | | | | | | | |
| GuideDesign90_ | TATA | ABE_NGG_ | spCas9 | TTGTATA | 3097 | UUGUAUAA | 3403 | TGG |
| ABE_NGG_ | box | 20 nt_3- | ABE | AAAGGCT | | AAGGCUGG | | |
| 20 nt_3- | | 9_002 | | GGGGGCT | | GGGC | | |
| 9_002_+_ | | | | GG | | | | |
| 31946061_spCas9 | | | | | | | | |
| GuideDesign90_ | TATA | ABE_NGG_ | spCas9 | TTGTATA | 3098 | UUGUAUAA | 3404 | TGG |
| ABE_NGG_ | box | 20 nt_3- | IBE | AAAGGCT | | AAGGCUGG | | |
| 20 nt_3- | | 12_018 | | GGGGGCT | | GGGC | | |
| 12_018_+_ | | | | GG | | | | |
| 31946061_spCas9 | | | | | | | | |
| GuideDesign90_ | TATA | ABE_NGG_ | SpCas9 | TTGTATA | 3099 | UUGUAUAA | 3405 | TGG |
| ABE_NGG_ | box | 20 nt_13- | IBE_ | AAAGGCT | | AAGGCUGG | | |
| 20 nt_13- | | 16_032 | expanded | GGGGGCT | | GGGC | | |
| 16_032_+_ | | | ABE | GG | | | | |
| 31946061_SpCas9 | | | | | | | | |
| Guide_Design90_ | TATA | CBE_NGG_ | spCas9 | TTGTATA | 3100 | UUGUAUAA | 3406 | TGG |
| CBE_NGG_ | box | 20 nt_4- | CBE | AAAGGCT | | AAGGCUGG | | |
| 20 nt_4- | | 9_003 | | GGGGGCT | | GGGC | | |
| 9_003_+_ | | | | GG | | | | |
| 31946061_spCas9 | | | | | | | | |
| GuideDesign90_ | TATA | CBE_NGG_ | spCas9 | TTGTATA | 3101 | UUGUAUAA | 3407 | TGG |
| CBE_NGG_ | box | 20 nt_3- | NGG | AAAGGCT | | AAGGCUGG | | |
| 20 nt_3- | | 9_029 | CBE | GGGGGCT | | GGGC | | |
| 9_029_+_ | | | | GG | | | | |
| 31946061_spCas9 | | | | | | | | |
| GuideDesign90_ | TATA | CBE_NGG_ | SpCas9 | TTGTATA | 3102 | UUGUAUAA | 3408 | TGG |
| CBE_NGG_ | box | 20 nt_3- | IBE | AAAGGCT | | AAGGCUGG | | |
| 20 nt_3- | | 16_033 | CBE | GGGGGCT | | GGGC | | |
| 16_033_+_ | | | | GG | | | | |
| 31946061_SpCas9 | | | | | | | | |
| GuideDesign90_ | TATA | ABE_NG_ | spCas9 | TTGTATA | 3103 | UUGUAUAA | 3409 | TG |
| ABE_NG_ | box | 20 nt_3- | NG | AAAGGCT | | AAGGCUGG | | |
| 20 nt_3- | | 9_027 | ABE | GGGGGCT | | GGGC | | |
| 9_027_+_ | | | | G | | | | |
| 31946061_spCas9 | | | | | | | | |
| GuideDesign90_ | TATA | CBE_NG_ | spCas9 | TTGTATA | 3104 | UUGUAUAA | 3410 | TG |
| CBE_NG_ | box | 20 nt_4- | NG | AAAGGCT | | AAGGCUGG | | |
| 20 nt_4- | | 9_028 | CBE | GGGGGCT | | GGGC | | |
| 9_028_+_ | | | | G | | | | |
| 31946061_spCas9 | | | | | | | | |
| GuideDesign90_ | TATA | ABE_NRN_ | spCas9 | TTGTATA | 3105 | UUGUAUAA | 3411 | TGG |
| ABE_NRN_ | box | 20 nt_3- | NRN | AAAGGCT | | AAGGCUGG | | |
| 20 nt_3- | | 9_021 | ABE | GGGGGCT | | GGGC | | |
| 9_021_+_ | | | | GG | | | | |
| 31946061_spCas9 | | | | | | | | |
| GuideDesign90_ | TATA | CBE_NRN_ | spCas9 | TTGTATA | 3106 | UUGUAUAA | 3412 | TGG |
| CBE_NRN_ | box | 20 nt_3- | NRN | AAAGGCT | | AAGGCUGG | | |
| 20 nt_3- | | 9_022 | CBE | GGGGGCT | | GGGC | | |
| 9_022_+_ | | | | GG | | | | |
| 31946061_spCas9 | | | | | | | | |
| GuideDesign90_ | TATA | MBE_NRN_ | spCas9 | TTGTATA | 3107 | UUGUAUAA | 3413 | TGG |
| MBE_NRN_ | box | 20 nt_3- | NRN | AAAGGCT | | AAGGCUGG | | |
| 20 nt_3- | | 9_023 | MBE | GGGGGCT | | GGGC | | |
| 9_023_+_ | | | | GG | | | | |
| 31946061_spCas9 | | | | | | | | |
| GuideDesign90_ | TATA | CBE_NRRH_ | spCas9 | TTGTATA | 3108 | UUGUAUAA | 3414 | TGGT |
| CBE_NRRH_ | box | 20 nt_3- | NRRH | AAAGGCT | | AAGGCUGG | | |
| 20 nt_3- | | 9_031 | CBE | GGGGGCT | | GGGC | | |
| 9_031_+_ | | | | GGT | | | | |
| 31946061_spCas9 | | | | | | | | |
| GuideDesign90_ | TATA | ABE_NGC_ | spCas9 | TTTTATA | 3109 | UUUUAUAC | 3415 | GGC |
| ABE_NGC_ | box | 20 nt_3- | NGC | CAATCTG | | AAUCUGUG | | |
| 20 nt_3- | | 9_008 | ABE | TGTTCTG | | UUCU | | |
| 9_008_-_ | | | | GC | | | | |
| 31946048_ | | | | | | | | |
| spCas9 | | | | | | | | |
| GuideDesign90_ | TATA | ABE_NGC_ | spCas9 | TTTTATA | 3110 | UUUUAUAC | 3416 | GGC |
| ABE_NGC_ | box | 20 nt_3- | NGC | CAATCTG | | AAUCUGUG | | |
| 20 nt_3- | | 12_020 | IBE | TGTTCTG | | UUCU | | |
| 12_020_-_ | | | | GC | | | | |
| 31946048_ | | | | | | | | |
| spCas9 | | | | | | | | |
| GuideDesign90_ | TATA | CBE_NGC_ | spCas9 | TTTTATA | 3111 | UUUUAUAC | 3417 | GGC |
| CBE_NGC_ | box | 20 nt_4- | NGC | CAATCTG | | AAUCUGUG | | |
| 20 nt_4- | | 9_009 | CBE | TGTTCTG | | UUCU | | |
| 9_009_-_ | | | | GC | | | | |
| 31946048_ | | | | | | | | |
| spCas9 | | | | | | | | |
| GuideDesign90_ | TATA | ABE_NG_ | spCas9 | TTTTATA | 3112 | UUUUAUAC | 3418 | GG |
| ABE_NG_20 nt_ | box | 20 nt_3- | NG | CAATCTG | | AAUCUGUG | | |
| 3-9_027_-_ | | 9_027 | ABE | TGTTCTG | | UUCU | | |
| 31946049_ | | | | G | | | | |
| spCas9 | | | | | | | | |
| GuideDesign90_ | TATA | CBE_NG_ | spCas9 | TTTTATA | 3113 | UUUUAUAC | 3419 | GG |
| CBE_NG_20 nt_ | box | 20 nt_4- | NG | CAATCTG | | AAUCUGUG | | |
| 4-9_028_-_ | | 9_028 | CBE | TGTTCTG | | UUCU | | |
| 31946049_ | | | | G | | | | |
| spCas9 | | | | | | | | |
| GuideDesign90_ | TATA | ABE_NRN_ | spCas9 | TTTTATA | 3114 | UUUUAUAC | 3420 | GGC |
| ABE_NRN_ | box | 20 nt_3- | NRN | CAATCTG | | AAUCUGUG | | |
| 20 nt_3- | | 9_021 | ABE | TGTTCTG | | UUCU | | |
| 9_021_-_ | | | | GC | | | | |
| 31946048_ | | | | | | | | |
| spCas9 | | | | | | | | |
| GuideDesign90_ | TATA | CBE_NRN_ | spCas9 | TTTTATA | 3115 | UUUUAUAC | 3421 | GGC |
| CBE_NRN_ | box | 20 nt_3- | NRN | CAATCTG | | AAUCUGUG | | |
| 20 nt_3- | | 9_022 | CBE | TGTTCTG | | UUCU | | |
| 9_022_-_ | | | | GC | | | | |
| 31946048_ | | | | | | | | |
| spCas9 | | | | | | | | |
| GuideDesign90_ | TATA | MBE_NRN_ | spCas9 | TTTTATA | 3116 | UUUUAUAC | 3422 | GGC |
| MBE_NRN_ | box | 20 nt_3- | NRN | CAATCTG | | AAUCUGUG | | |
| 20 nt_3- | | 9_023 | MBE | TGTTCTG | | UUCU | | |
| 9_023_-_ | | | | GC | | | | |
| 31946048_ | | | | | | | | |
| spCas9 | | | | | | | | |
| GuideDesign90_ | TATA | ABE_NRCH_ | spCas9 | TTTTATA | 3117 | UUUUAUAC | 3423 | GGCA |
| ABE_NRC_ | box | 20 nt_3- | NRCH | CAATCTG | | AAUCUGUG | | |
| H_20 nt_3- | | 9_024 | ABE | TGTTCTG | | UUCU | | |
| 9_024_-_ | | | | GCA | | | | |
| 31946047_ | | | | | | | | |
| spCas9 | | | | | | | | |
| GuideDesign90_ | TATA | CBE_NRCH_ | spCas9 | TTTTATA | 3118 | UUUUAUAC | 3424 | GGCA |
| CBE_NRCH_ | box | 20 nt_3- | NRCH | CAATCTG | | AAUCUGUG | | |
| 20 nt_3- | | 9_030 | CBE | TGTTCTG | | UUCU | | |
| 9_030_-_ | | | | GCA | | | | |
| 31946047_ | | | | | | | | |
| spCas9 | | | | | | | | |
| GuideDesign90_ | Start | ABE_NRN_ | spCas9 | AACGCCA | 3119 | AACGCCAU | 3425 | CAG |
| ABE_NRN_ | codon | 20 nt_3- | NRN | TGGGGAG | | GGGGAGCA | | |
| 20 nt_3- | | 9_021 | ABE | CAATCTC | | AUCU | | |
| 9_021_+_ | | | | AG | | | | |
| 31946215_spCas9 | | | | | | | | |
| GuideDesign90_ | Start | MBE_NRN_ | spCas9 | AACGCCA | 3120 | AACGCCAU | 3426 | CAG |
| MBE_NRN_ | codon | 20 nt_3- | NRN | TGGGGAG | | GGGGAGCA | | |
| 20 nt_3- | | 9_023 | MBE | CAATCTC | | AUCU | | |
| 9_023_+_ | | | | AG | | | | |
| 31946215_spCas9 | | | | | | | | |
| GuideDesign90_ | Start | ABE_NGC_ | spCas9 | ACGCCAT | 3121 | ACGCCAUG | 3427 | AGC |
| ABE_NGC_ | codon | 20 nt_3- | NGC | GGGGAGC | | GGGAGCAA | | |
| 20 nt_3- | | 9_008 | ABE | AATCTCA | | UCUC | | |
| 9_008_+_ | | | | GC | | | | |
| 31946216_spCas9 | | | | | | | | |
| GuideDesign90_ | Start | ABE_NGC_ | spCas9 | ACGCCAT | 3122 | ACGCCAUG | 3428 | AGC |
| ABE_NGC_ | codon | 20 nt_3- | NGC | GGGGAGC | | GGGAGCAA | | |
| 20 nt_3- | | 12_020 | IBE | AATCTCA | | UCUC | | |
| 12_020_+_ | | | | GC | | | | |
| 31946216_spCas9 | | | | | | | | |
| GuideDesign90_ | Start | ABE_NG_ | spCas9 | ACGCCAT | 3123 | ACGCCAUG | 3429 | AG |
| ABE_NG_ | codon | 20 nt_3- | NG | GGGGAGC | | GGGAGCAA | | |
| 20 nt_3- | | 9_027 | ABE | AATCTCA | | UCUC | | |
| 9_027_+_ | | | | G | | | | |
| 31946216_spCas9 | | | | | | | | |
| GuideDesign90_ | Start | ABE_NRN_ | spCas9 | ACGCCAT | 3124 | ACGCCAUG | 3430 | AGC |
| ABE_NRN_ | codon | 20 nt_3- | NRN | GGGGAGC | | GGGAGCAA | | |
| 20 nt_3- | | 9_021 | ABE | AATCTCA | | UCUC | | |
| 9_021_+_ | | | | GC | | | | |
| 31946216_spCas9 | | | | | | | | |
| GuideDesign90_ | Start | MBE_NRN_ | spCas9 | ACGCCAT | 3125 | ACGCCAUG | 3431 | AGC |
| MBE_NRN_ | codon | 20 nt_3- | NRN | GGGGAGC | | GGGAGCAA | | |
| 20 nt_3- | | 9_023 | MBE | AATCTCA | | UCUC | | |
| 9_023_+_ | | | | GC | | | | |
| 31946216_spCas9 | | | | | | | | |
| GuideDesign90_ | Start | ABE_NRCH_ | spCas9 | ACGCCAT | 3126 | ACGCCAUG | 3432 | AGCC |
| ABE_NRC_ | codon | 20 nt_3- | NRCH | GGGGAGC | | GGGAGCAA | | |
| H_20 nt_3- | | 9_024 | ABE | AATCTCA | | UCUC | | |
| 9_024_+_ | | | | GCC | | | | |
| 31946216_spCas9 | | | | | | | | |
| GuideDesign90_ | Start | CBE_NGA_ | spCas9 | AGATTGC | 3127 | AGAUUGCU | 3433 | GGA |
| CBE_NGA_ | codon | 20 nt_3- | IBE_ | TCCCCAT | | CCCCAUGG | | |
| 20 nt_3- | | 16_035 | extended | GGCGTTG | | CGUU | | |
| 16_035_- | | | CBE | GA | | | | |
| 31946212_ | | | | | | | | |
| spCas9 | | | | | | | | |
| GuideDesign90_ | Start | ABE_VTTN_ | cas12b | CTTCCAA | 3128 | CAACGCCA | 3434 | CTTC |
| ABE_VTTN_ | codon | 22 nt_5- | ABE | CGCCATG | | UGGGGAGC | | |
| 22 nt_5- | | 9_017 | | GGGAGCA | | AAUCUC | | |
| 9_017_+_ | | | | ATCTC | | | | |
| 31946210_cas12b | | | | | | | | |
| GuideDesign90_ | Start | ABE_NG_ | spCas9 | CCATGGC | 3129 | CCAUGGCG | 3435 | AG |
| ABE_NG_20 nt_ | codon | 20 nt_3- | NG | GTTGGAA | | UUGGAAGG | | |
| 3-9_027_-_ | | 9_027 | ABE | GGCAGGA | | CAGG | | |
| 31946203_ | | | | G | | | | |
| spCas9 | | | | | | | | |
| GuideDesign90_ | Start | ABE_NRN_ | spCas9 | CCATGGC | 3130 | CCAUGGCG | 3436 | AGA |
| ABE_NRN_ | codon | 20 nt_3- | NRN | GTTGGAA | | UUGGAAGG | | |
| 20 nt_3- | | 9_021 | ABE | GGCAGGA | | CAGG | | |
| 9_021_-_ | | | | GA | | | | |
| 31946202_ | | | | | | | | |
| spCas9 | | | | | | | | |
| GuideDesign90_ | Start | MBE_NRN_ | spCas9 | CCATGGC | 3131 | CCAUGGCG | 3437 | AGA |
| MBE_NRN_ | codon | 20 nt_3- | NRN | GTTGGAA | | UUGGAAGG | | |
| 20 nt_3- | | 9_023 | MBE | GGCAGGA | | CAGG | | |
| 9_023_-_ | | | | GA | | | | |
| 31946202_ | | | | | | | | |
| spCas9 | | | | | | | | |
| GuideDesign90_ | Start | ABE_NGA_ | spCas9 | CCATGGC | 3132 | CCAUGGCG | 3438 | AGA |
| ABE_NGA_ | codon | 20 nt_3- | VRQR | GTTGGAA | | UUGGAAGG | | |
| 20 nt_3- | | 12_019 | IBE | GGCAGGA | | CAGG | | |
| 12_019_-_ | | | | GA | | | | |
| 31946202_ | | | | | | | | |
| spCas9 | | | | | | | | |
| GuideDesign90_ | Start | ABE_NRN_ | spCas9 | CCCATGG | 3133 | CCCAUGGC | 3439 | GAG |
| ABE_NRN_ | codon | 20 nt_3- | NRN | CGTTGGA | | GUUGGAAG | | |
| 20 nt_3- | | 9_021 | ABE | AGGCAGG | | GCAG | | |
| 9_021_-_ | | | | AG | | | | |
| 31946203_ | | | | | | | | |
| spCas9 | | | | | | | | |
| GuideDesign90_ | Start | CBE_NRN_ | spCas9 | CCCATGG | 3134 | CCCAUGGC | 3440 | GAG |
| CBE_NRN_ | codon | 20 nt_3- | NRN | CGTTGGA | | GUUGGAAG | | |
| 20 nt_3- | | 9_022 | CBE | AGGCAGG | | GCAG | | |
| 9_022_-_ | | | | AG | | | | |
| 31946203_ | | | | | | | | |
| spCas9 | | | | | | | | |
| GuideDesign90_ | Start | MBE_NRN_ | spCas9 | CCCATGG | 3135 | CCCAUGGC | 3441 | GAG |
| MBE_NRN_ | codon | 20 nt_3- | NRN | CGTTGGA | | GUUGGAAG | | |
| 20 nt_3- | | 9_023 | MBE | AGGCAGG | | GCAG | | |
| 9_023_-_ | | | | AG | | | | |
| 31946203_ | | | | | | | | |
| spCas9 | | | | | | | | |
| GuideDesign90_ | Start | CBE_NRRH_ | spCas9 | CCCATGG | 3136 | CCCAUGGC | 3442 | GAGA |
| CBE_NRR_ | codon | 20 nt_3- | NRRH | CGTTGGA | | GUUGGAAG | | |
| H_20 nt_3- | | 9_031 | CBE | AGGCAGG | | GCAG | | |
| 9_031_-_ | | | | AGA | | | | |
| 31946202_ | | | | | | | | |
| spCas9 | | | | | | | | |
| GuideDesign90_ | Start | CBE_NGA_ | spCas9 | CCCCATG | 3137 | CCCCAUGG | 3443 | GGA |
| CBE_NGA_ | codon | 20 nt_3- | IBE_ | GCGTTGG | | CGUUGGAA | | |
| 20 nt_3- | | 16_035 | extended | AAGGCAG | | GGCA | | |
| 16_035_-_ | | | CBE | GA | | | | |
| 31946204_ | | | | | | | | |
| spCas9 | | | | | | | | |
| GuideDesign90_ | Start | ABE_NG_ | spCas9 | CCCCATG | 3138 | CCCCAUGG | 3444 | GG |
| ABE_NG_20 nt_ | codon | 20 nt_3- | NG | GCGTTGG | | CGUUGGAA | | |
| 3-9_027_-_ | | 9_027 | ABE | AAGGCAG | | GGCA | | |
| 31946205_ | | | | G | | | | |
| spCas9 | | | | | | | | |
| GuideDesign90_ | Start | CBE_NG_ | spCas9 | CCCCATG | 3139 | CCCCAUGG | 3445 | GG |
| CBE_NG_20 nt_ | codon | 20 nt_4- | NG | GCGTTGG | | CGUUGGAA | | |
| 4-9_028_-_ | | 9_028 | CBE | AAGGCAG | | GGCA | | |
| 31946205_ | | | | G | | | | |
| spCas9 | | | | | | | | |
| GuideDesign90_ | Start | ABE_NRN_ | spCas9 | CCCCATG | 3140 | CCCCAUGG | 3446 | GGA |
| ABE_NRN_ | codon | 20 nt_3- | NRN | GCGTTGG | | CGUUGGAA | | |
| 20 nt_3- | | 9_021 | ABE | AAGGCAG | | GGCA | | |
| 9_021_-_ | | | | GA | | | | |
| 31946204_ | | | | | | | | |
| spCas9 | | | | | | | | |
| GuideDesign90_ | Start | CBE_NRN_ | spCas9 | CCCCATG | 3141 | CCCCAUGG | 3447 | GGA |
| CBE_NRN_ | codon | 20 nt_3- | NRN | GCGTTGG | | CGUUGGAA | | |
| 20 nt_3- | | 9_022 | CBE | AAGGCAG | | GGCA | | |
| 9_022_-_ | | | | GA | | | | |
| 31946204_ | | | | | | | | |
| spCas9 | | | | | | | | |
| GuideDesign90_ | Start | MBE_NRN_ | spCas9 | CCCCATG | 3142 | CCCCAUGG | 3448 | GGA |
| MBE_NRN_ | codon | 20 nt_3- | NRN | GCGTTGG | | CGUUGGAA | | |
| 20 nt_3- | | 9_023 | MBE | AAGGCAG | | GGCA | | |
| 9_023_-_ | | | | GA | | | | |
| 31946204_ | | | | | | | | |
| spCas9 | | | | | | | | |
| GuideDesign90_ | Start | ABE_NGA_ | spCas9 | CCCCATG | 3143 | CCCCAUGG | 3449 | GGA |
| ABE_NGA_ | codon | 20 nt_3- | VRQR | GCGTTGG | | CGUUGGAA | | |
| 20 nt_3- | | 12_019 | IBE | AAGGCAG | | GGCA | | |
| 12_019_-_ | | | | GA | | | | |
| 31946204_ | | | | | | | | |
| spCas9 | | | | | | | | |
| GuideDesign90_ | Start | CBE_NGA_ | spCas9 | CCCCATG | 3144 | CCCCAUGG | 3450 | GGA |
| CBE_NGA_ | codon | 20 nt_4- | VRQR | GCGTTGG | | CGUUGGAA | | |
| 20 nt_4- | | 9_006 | CBE | AAGGCAG | | GGCA | | |
| 9_006_-_ | | | | GA | | | | |
| 31946204_ | | | | | | | | |
| spCas9 | | | | | | | | |
| GuideDesign90_ | Start | ABE_NRN_ | spCas9 | CTCCCCA | 3145 | CUCCCCAU | 3451 | CAG |
| ABE_NRN_ | codon | 20 nt_3- | NRN | TGGCGTT | | GGCGUUGG | | |
| 20 nt_3- | | 9_021 | ABE | GGAAGGC | | AAGG | | |
| 9_021_-_ | | | | AG | | | | |
| 31946206_ | | | | | | | | |
| spCas9 | | | | | | | | |
| GuideDesign90_ | Start | CBE_NRN_ | spCas9 | CTCCCCA | 3146 | CUCCCCAU | 3452 | CAG |
| CBE_NRN_ | codon | 20 nt_3- | NRN | TGGCGTT | | GGCGUUGG | | |
| 20 nt_3- | | 9_022 | CBE | GGAAGGC | | AAGG | | |
| 9_022_-_ | | | | AG | | | | |
| 31946206_ | | | | | | | | |
| spCas9 | | | | | | | | |
| GuideDesign90_ | Start | MBE_NRN_ | spCas9 | CTCCCCA | 3147 | CUCCCCAU | 3453 | CAG |
| MBE_NRN_ | codon | 20 nt_3- | NRN | TGGCGTT | | GGCGUUGG | | |
| 20 nt_3- | | 9_023 | MBE | GGAAGGC | | AAGG | | |
| 9_023_-_ | | | | AG | | | | |
| 31946206_ | | | | | | | | |
| spCas9 | | | | | | | | |
| GuideDesign90_ | Start | ABE_VTTN_ | cas12b | ATTGCTC | 3148 | CUCCCCAU | 3454 | ATTG |
| ABE_VTTN_ | codon | 22 nt_5- | ABE | CCCATGG | | GGCGUUGG | | |
| 22 nt_5- | | 9_017 | | CGTTGGA | | AAGGCA | | |
| 9_017_-_ | | | | AGGCA | | | | |
| 31946207_ | | | | | | | | |
| cas12b | | | | | | | | |
| GuideDesign90_ | Start | CBE_NGG_ | SpCas9 | GAGATTG | 3149 | GAGAUUGC | 3455 | TGG |
| CBE_NGG_ | codon | 20 nt_3- | IBE | CTCCCCA | | UCCCCAUG | | |
| 20 nt_3- | | 16_033 | CBE | TGGCGTT | | GCGU | | |
| 16_033_-_ | | | | GG | | | | |
| 31946213_ | | | | | | | | |
| SpCas9 | | | | | | | | |
| GuideDesign90_ | Start | ABE_NGG_ | spCas9 | TCCCCAT | 3150 | UCCCCAUG | 3456 | AGG |
| ABE_NGG_ | codon | 20 nt_3- | ABE | GGCGTTG | | GCGUUGGA | | |
| 20 nt_3- | | 9_002 | | GAAGGCA | | AGGC | | |
| 9_002_-_ | | | | GG | | | | |
| 31946205_ | | | | | | | | |
| spCas9 | | | | | | | | |
| GuideDesign90_ | Start | ABE_NG_ | spCas9 | TCCCCAT | 3151 | UCCCCAUG | 3457 | AG |
| ABE_NG_20 nt_ | codon | 20 nt_3- | NG | GGCGTTG | | GCGUUGGA | | |
| 3-9_027_-_ | | 9_027 | ABE | GAAGGCA | | AGGC | | |
| 31946206_ | | | | G | | | | |
| spCas9 | | | | | | | | |
| GuideDesign90_ | Start | CBE_NG_ | spCas9 | TCCCCAT | 3152 | UCCCCAUG | 3458 | AG |
| CBE_NG_20 nt_ | codon | 20 nt_4- | NG | GGCGTTG | | GCGUUGGA | | |
| 4-9_028_-_ | | 9_028 | CBE | GAAGGCA | | AGGC | | |
| 31946206_ | | | | G | | | | |
| spCas9 | | | | | | | | |
| GuideDesign90_ | Start | ABE_NGG_ | spCas9 | TCCCCAT | 3153 | UCCCCAUG | 3459 | AGG |
| ABE_NGG_ | codon | 20 nt_3- | IBE | GGCGTTG | | GCGUUGGA | | |
| 20 nt_3- | | 12_018 | | GAAGGCA | | AGGC | | |
| 12_018_-_ | | | | GG | | | | |
| 31946205_ | | | | | | | | |
| spCas9 | | | | | | | | |
| GuideDesign90_ | Start | ABE_NRN_ | spCas9 | TCCCCAT | 3154 | UCCCCAUG | 3460 | AGG |
| ABE_NRN_ | codon | 20 nt_3- | NRN | GGCGTTG | | GCGUUGGA | | |
| 20 nt_3- | | 9_021 | ABE | GAAGGCA | | AGGC | | |
| 9_021_-_ | | | | GG | | | | |
| 31946205_ | | | | | | | | |
| spCas9 | | | | | | | | |
| GuideDesign90_ | Start | CBE_NRN_ | spCas9 | TCCCCAT | 3155 | UCCCCAUG | 3461 | AGG |
| CBE_NRN_ | codon | 20 nt_3- | NRN | GGCGTTG | | GCGUUGGA | | |
| 20 nt_3- | | 9_022 | CBE | GAAGGCA | | AGGC | | |
| 9_022_-_ | | | | GG | | | | |
| 31946205_ | | | | | | | | |
| spCas9 | | | | | | | | |
| GuideDesign90_ | Start | MBE_NRN_ | spCas9 | TCCCCAT | 3156 | UCCCCAUG | 3462 | AGG |
| MBE_NRN_ | codon | 20 nt_3- | NRN | GGCGTTG | | GCGUUGGA | | |
| 20 nt_3- | | 9_023 | MBE | GAAGGCA | | AGGC | | |
| 9_023_-_ | | | | GG | | | | |
| 31946205_ | | | | | | | | |
| spCas9 | | | | | | | | |
| GuideDesign90_ | Start | CBE_NRRH_ | spCas9 | TCCCCAT | 3157 | UCCCCAUG | 3463 | AGGA |
| CBE_NRR_ | codon | 20 nt_3- | NRRH | GGCGTTG | | GCGUUGGA | | |
| H_20 nt_3- | | 9_031 | CBE | GAAGGCA | | AGGC | | |
| 9_031_-_ | | | | GGA | | | | |
| 31946204_ | | | | | | | | |
| spCas9 | | | | | | | | |
| GuideDesign90_ | Start | CBE_NGG_ | spCas9 | TCCCCAT | 3158 | UCCCCAUG | 3464 | AGG |
| CBE_NGG_ | codon | 20 nt_4- | CBE | GGCGTTG | | GCGUUGGA | | |
| 20 nt_4- | | 9_003 | | GAAGGCA | | AGGC | | |
| 9_003_-_ | | | | GG | | | | |
| 31946205_ | | | | | | | | |
| spCas9 | | | | | | | | |
| GuideDesign90_ | Start | CBE_NGG_ | spCas9 | TCCCCAT | 3159 | UCCCCAUG | 3465 | AGG |
| CBE_NGG_ | codon | 20 nt_3- | NGG | GGCGTTG | | GCGUUGGA | | |
| 20 nt_3- | | 9_029 | CBE | GAAGGCA | | AGGC | | |
| 9_029_-_ | | | | GG | | | | |
| 31946205_ | | | | | | | | |
| spCas9 | | | | | | | | |
| GuideDesign90_ | Start | CBE_NGG_ | SpCas9 | TCCCCAT | 3160 | UCCCCAUG | 3466 | AGG |
| CBE_NGG_ | codon | 20 nt_3- | IBE | GGCGTTG | | GCGUUGGA | | |
| 20 nt_3- | | 16_033 | CBE | GAAGGCA | | AGGC | | |
| 16_033_-_ | | | | GG | | | | |
| 31946205_ | | | | | | | | |
| SpCas9 | | | | | | | | |
| GuideDesign90_ | Start | ABE_NGC_ | spCas9 | TGCTCCC | 3161 | UGCUCCCC | 3467 | GGC |
| ABE_NGC_ | codon | 20 nt_3- | NGC | CATGGCG | | AUGGCGUU | | |
| 20 nt_3- | | 12_020 | IBE | TTGGAAG | | GGAA | | |
| 12_020_-_ | | | | GC | | | | |
| 31946208_ | | | | | | | | |
| spCas9 | | | | | | | | |
| GuideDesign90_ | Start | CBE_NGC_ | spCas9 | TGCTCCC | 3162 | UGCUCCCC | 3468 | GGC |
| CBE_NGC_ | codon | 20 nt_4- | NGC | CATGGCG | | AUGGCGUU | | |
| 20 nt_4- | | 9_009 | CBE | TTGGAAG | | GGAA | | |
| 9_009_-_ | | | | GC | | | | |
| 31946208_ | | | | | | | | |
| spCas9 | | | | | | | | |
| GuideDesign90_ | Start | ABE_NG_ | spCas9 | TGCTCCC | 3163 | UGCUCCCC | 3469 | GG |
| ABE_NG_20 nt_ | codon | 20 nt_3- | NG | CATGGCG | | AUGGCGUU | | |
| 3-9_027_-_ | | 9_027 | ABE | TTGGAAG | | GGAA | | |
| 31946209_ | | | | G | | | | |
| spCas9 | | | | | | | | |
| GuideDesign90_ | Start | CBE_NG_ | spCas9 | TGCTCCC | 3164 | UGCUCCCC | 3470 | GG |
| CBE_NG_20 nt_ | codon | 20 nt_4- | NG | CATGGCG | | AUGGCGUU | | |
| 4-9_028_-_ | | 9_028 | CBE | TTGGAAG | | GGAA | | |
| 31946209_ | | | | G | | | | |
| spCas9 | | | | | | | | |
| GuideDesign90_ | Start | ABE_NRN_ | spCas9 | TGCTCCC | 3165 | UGCUCCCC | 3471 | GGC |
| ABE_NRN_ | codon | 20 nt_3- | NRN | CATGGCG | | AUGGCGUU | | |
| 20 nt_3- | | 9_021 | ABE | TTGGAAG | | GGAA | | |
| 9_021_-_ | | | | GC | | | | |
| 31946208_ | | | | | | | | |
| spCas9 | | | | | | | | |
| GuideDesign90_ | Start | CBE_NRN_ | spCas9 | TGCTCCC | 3166 | UGCUCCCC | 3472 | GGC |
| CBE_NRN_ | codon | 20 nt_3- | NRN | CATGGCG | | AUGGCGUU | | |
| 20 nt_3- | | 9_022 | CBE | TTGGAAG | | GGAA | | |
| 9_022_-_ | | | | GC | | | | |
| 31946208_ | | | | | | | | |
| spCas9 | | | | | | | | |
| GuideDesign90_ | Start | MBE_NRN_ | spCas9 | TGCTCCC | 3167 | UGCUCCCC | 3473 | GGC |
| MBE_NRN_ | codon | 20 nt_3- | NRN | CATGGCG | | AUGGCGUU | | |
| 20 nt_3- | | 9_023 | MBE | TTGGAAG | | GGAA | | |
| 9_023_-_ | | | | GC | | | | |
| 31946208_ | | | | | | | | |
| spCas9 | | | | | | | | |
| GuideDesign90_ | Start | ABE_NRCH_ | spCas9 | TGCTCCC | 3168 | UGCUCCCC | 3474 | GGCA |
| ABE_NRCH_ | codon | 20 nt_3- | NRCH | CATGGCG | | AUGGCGUU | | |
| 20 nt_3- | | 9_024 | ABE | TTGGAAG | | GGAA | | |
| 9_024_-_ | | | | GCA | | | | |
| 31946207_ | | | | | | | | |
| spCas9 | | | | | | | | |
| GuideDesign90_ | Start | CBE_NRCH_ | spCas9 | TGCTCCC | 3169 | UGCUCCCC | 3475 | GGCA |
| CBE_NRCH_ | codon | 20 nt_3- | NRCH | CATGGCG | | AUGGCGUU | | |
| 20 nt_3- | | 9_030 | CBE | TTGGAAG | | GGAA | | |
| 9_030_-_ | | | | GCA | | | | |
| 31946207_ | | | | | | | | |
| spCas9 | | | | | | | | |
| GuideDesign90_ | Start | CBE_NG_ | spCas9 | TTGCTCC | 3170 | UUGCUCCC | 3476 | AG |
| CBE_NG_20 nt_ | codon | 20 nt_4- | NG | CCATGGC | | CAUGGCGU | | |
| 4-9_028_-_ | | 9_028 | CBE | GTTGGAA | | UGGA | | |
| 31946210_ | | | | G | | | | |
| spCas9 | | | | | | | | |
| GuideDesign90_ | Start | CBE_NRN_ | spCas9 | TTGCTCC | 3171 | UUGCUCCC | 3477 | AGG |
| CBE_NRN_ | codon | 20 nt_3- | NRN | CCATGGC | | CAUGGCGU | | |
| 20 nt_3- | | 9_022 | CBE | GTTGGAA | | UGGA | | |
| 9_022_-_ | | | | GG | | | | |
| 31946209_ | | | | | | | | |
| spCas9 | | | | | | | | |
| GuideDesign90_ | Start | MBE_NRN_ | spCas9 | TTGCTCC | 3172 | UUGCUCCC | 3478 | AGG |
| MBE_NRN_ | codon | 20 nt_3- | NRN | CCATGGC | | CAUGGCGU | | |
| 20 nt_3- | | 9_023 | MBE | GTTGGAA | | UGGA | | |
| 9_023_-_ | | | | GG | | | | |
| 31946209_ | | | | | | | | |
| spCas9 | | | | | | | | |
| GuideDesign90_ | Start | CBE_NRRH_ | spCas9 | TTGCTCC | 3173 | UUGCUCCC | 3479 | AGGC |
| CBE_NRRH_ | codon | 20 nt_3- | NRRH | CCATGGC | | CAUGGCGU | | |
| 20 nt_3- | | 9_031 | CBE | GTTGGAA | | UGGA | | |
| 9_031_-_ | | | | GGC | | | | |
| 31946208_ | | | | | | | | |
| spCas9 | | | | | | | | |
| GuideDesign90_ | Start | CBE_NGG_ | spCas9 | TTGCTCC | 3174 | UUGCUCCC | 3480 | AGG |
| CBE_NGG_ | codon | 20 nt_4- | CBE | CCATGGC | | CAUGGCGU | | |
| 20 nt_4- | | 9_003 | | GTTGGAA | | UGGA | | |
| 9_003_-_ | | | | GG | | | | |
| 31946209_ | | | | | | | | |
| spCas9 | | | | | | | | |
| GuideDesign90_ | Start | CBE_NGG_ | spCas9 | TTGCTCC | 3175 | UUGCUCCC | 3481 | AGG |
| CBE_NGG_ | codon | 20 nt_3- | NGG | CCATGGC | | CAUGGCGU | | |
| 20 nt_3- | | 9_029 | CBE | GTTGGAA | | UGGA | | |
| 9_029_-_ | | | | GG | | | | |
| 31946209_ | | | | | | | | |
| spCas9 | | | | | | | | |
| GuideDesign90_ | Start | CBE_NGG_ | SpCas9 | TTGCTCC | 3176 | UUGCUCCC | 3482 | AGG |
| CBE_NGG_ | codon | 20 nt_3- | IBE | CCATGGC | | CAUGGCGU | | |
| 20 nt_3- | | 16_033 | CBE | GTTGGAA | | UGGA | | |
| 16_033_-_ | | | | GG | | | | |
| 31946209_ | | | | | | | | |
| SpCas9 | | | | | | | | |

TABLE 2G
Representative spacer and target site sequences relating to altering a TATA
box or start codon of a complement factor B polynucleotide using base editing.

| Guide | | | | |
|---|---|---|---|---|
| poly- | | | | |
| nucleotide | Target site | Target site | | |
| name | name | description | Editor | Cas9 variant |
| gRNA3642 | TSBTx6496 | CFB_TATA_locus | ABE_NGG_20 nt_3- | spCas9 ABE |
| | | | 9_002 | |
| gRNA3643 | TSBTx6497 | CFB_TATA_locus | ABE_NGG_20 nt_3- | spCas9 ABE |
| | | | 9_002 | |
| gRNA3644 | TSBTx6498 | CFB_TATA_locus | ABE_NGG_20 nt_3- | spCas9 ABE |
| | | | 9_002 | |
| gRNA3645 | TSBTx6499 | CFB_TATA_locus | ABE_NGG_20 nt_3- | spCas9 ABE |
| | | | 9_002 | |
| gRNA3646 | TSBTx6500 | CFB_TATA_locus | ABE_NGG_20 nt_3- | spCas9 ABE |
| | | | 9_002 | |
| gRNA3647 | TSBTx6501 | CFB_TATA_locus | ABE_NGG_20 nt_3- | spCas9 ABE |
| | | | 9_002 | |
| gRNA3648 | TSBTx6502 | CFB_TATA_locus | ABE_NGG_20 nt_3- | spCas9 ABE |
| | | | 9_002 | |
| gRNA3649 | TSBTx6503 | CFB_TATA_locus | ABE_NGG_20 nt_3- | spCas9 ABE |
| | | | 9_002 | |
| gRNA3650 | TSBTx6504 | CFB_TATA_locus | ABE_NGG_20 nt_3- | spCas9 ABE |
| | | | 9_002 | |
| gRNA3651 | TSBTx6505 | CFB_TATA_locus | ABE_NGG_20 nt_3- | spCas9 ABE |
| | | | 9_002 | |
| gRNA3652 | TSBTx6506 | CFB_TATA_locus | ABE_NGA_20 nt_3- | spCas9 VRQR |
| | | | 9_005 | ABE |
| gRNA3653 | TSBTx6507 | CFB_TATA_locus | ABE_NGC_20 nt_3- | spCas9 NGC |
| | | | 9_008 | ABE |
| gRNA3654 | TSBTx6508 | CFB_TATA_locus | ABE_NGC_20 nt_3- | spCas9 NGC |
| | | | 9_008 | ABE |
| gRNA3655 | TSBTx6509 | CFB_TATA_locus | ABE_NNNRRT_21 nt_5- | saCas9 KKH |
| | | | 14_014 | ABE |
| gRNA3656 | TSBTx6510 | CFB_TATA_locus | ABE_NNNRRT_21 nt_5- | saCas9 KKH |
| | | | 14_014 | ABE |
| gRNA3657 | TSBTx6511 | Start codon | ABE_NGC_20 nt_3- | spCas9 NGC |
| | | | 9_008 | ABE |
| gRNA3658 | TSBTx6512 | Start codon | ABE_NGG_20 nt_3- | spCas9 IBE |
| | | | 12_018 | |
| gRNA3659 | TSBTx6513 | Start codon | ABE_NGG_20 nt_13- | SpCas9 |
| | | | 16_032 | IBE_expanded |
| | | | | ABE |
| gRNA3660 | TSBTx6514 | Start codon | ABE_NGA_20 nt_3- | spCas9 VRQR |
| | | | 9_005 | ABE |
| gRNA3661 | TSBTx6515 | Start codon | ABE_NGA_20 nt_3- | spCas9 VRQR |
| | | | 9_005 | ABE |
| gRNA3662 | TSBTx6516 | Start codon | ABE_NGA_20 nt_3- | SpCas9 IBE |
| | | | 12_034 | expanded ABE |

| Guide | | | | | |
|---|---|---|---|---|---|
| poly- | | | | | |
| nucleotide | | SEQ ID | PAM | | SEQ ID |
| name | Target sequence | NO | sequence | Spacer sequence | NO |
| gRNA3642 | CAGAACACAGATTGTATAAAAG | 3483 | NGG | CAGAACACAGAUUGUAU | 3504 |
| | G | | | AAA | |
| gRNA3643 | ACACAGATTGTATAAAAGGCTG | 3484 | NGG | ACACAGAUUGUAUAAAA | 3505 |
| | G | | | GGC | |
| gRNA3644 | CACAGATTGTATAAAAGGCTGG | 3485 | NGG | CACAGAUUGUAUAAAAG | 3506 |
| | G | | | GCU | |
| gRNA3645 | ACAGATTGTATAAAAGGCTGGG | 3486 | NGG | ACAGAUUGUAUAAAAGG | 3507 |
| | G | | | CUG | |
| gRNA3646 | CAGATTGTATAAAAGGCTGGGG | 3487 | NGG | CAGAUUGUAUAAAAGGC | 3508 |
| | G | | | UGG | |
| gRNA3647 | TATAAAAGGCTGGGGGCTGGTG | 3488 | NGG | UAUAAAAGGCUGGGGGC | 3509 |
| | G | | | UGG | |
| gRNA3648 | ATAAAAGGCTGGGGGCTGGTGG | 3489 | NGG | AUAAAAGGCUGGGGGCU | 3510 |
| | G | | | GGU | |
| gRNA3649 | TAAAAGGCTGGGGGCTGGTGGG | 3490 | NGG | UAAAAGGCUGGGGGCUG | 3511 |
| | G | | | GUG | |
| gRNA3650 | CTTTTATACAATCTGTGTTCTG | 3491 | NGG | CUUUUAUACAAUCUGUG | 3512 |
| | G | | | UUC | |
| gRNA3651 | AATCTGTGTTCTGGCACCTGCG | 3492 | NGG | AAUCUGUGUUCUGGCAC | 3513 |
| | G | | | CUG | |
| gRNA3652 | AAAAGGCTGGGGGCTGGTGGGG | 3493 | NGA | AAAAGGCUGGGGGCUGG | 3514 |
| | A | | | UGG | |
| gRNA3653 | AGAACACAGATTGTATAAAAGG | 3494 | NGC | AGAACACAGAUUGUAUA | 3515 |
| | C | | | AAA | |
| gRNA3654 | ATCTGTGTTCTGGCACCTGCGG | 3495 | NGC | AUCUGUGUUCUGGCACC | 3516 |
| | C | | | UGC | |
| gRNA3655 | AGATTGTATAAAAGGCTGGGGG | 3496 | NNNRR | AGAUUGUAUAAAAGGCU | 3517 |
| | CTGGT | | T | GGGG | |
| gRNA3656 | CCACCAGCCCCCAGCCTTTTAT | 3497 | NNNRR | CCACCAGCCCCCAGCCU | 3518 |
| | ACAAT | | T | UUUA | |
| gRNA3657 | TGCTCCCCATGGCGTTGGAAGG | 3498 | NGC | UGCUCCCCAUGGCGUUG | 3519 |
| | C | | | GAA | |
| gRNA3658 | TTGCTCCCCATGGCGTTGGAAG | 3499 | NGG | UUGCUCCCCAUGGCGUU | 3520 |
| | G | | | GGA | |
| gRNA3659 | GAGATTGCTCCCCATGGCGTTG | 3500 | NGG | GAGAUUGCUCCCCAUGG | 3521 |
| | G | | | CGU | |
| gRNA3660 | CCCCATGGCGTTGGAAGGCAGG | 3501 | NGA | CCCCAUGGCGUUGGAAG | 3522 |
| | A | | | GCA | |
| gRNA3661 | CCATGGCGTTGGAAGGCAGGAG | 3502 | NGA | CCAUGGCGUUGGAAGGC | 3523 |
| | A | | | AGG | |
| gRNA3662 | AGATTGCTCCCCATGGCGTTGG | 3503 | NGA | AGAUUGCUCCCCAUGGC | 3524 |
| | A | | | GUU | |

TABLE 2H
Representative spacer and target site sequences relating to disruption of a
complement factor B splice site using base editing.

| | | | | Target Sequence | | | |
|---|---|---|---|---|---|---|---|
| Guide | | | | (PAM is | | | Target |
| Poly- | Target | | SEQ | underlineed and | SEQ | Cyno | Site |
| nucleotide | Site | Spacer | ID | target nucleotide | ID | Cross (X) | Location |
| Name | Name | Sequence | NO | is in bold text) | NO | Reactivity | Description |
| gRNA1193 | TSBTx3826 | CCUCAGAU | 3525 | CCTCAGATGTCT | 3529 | Y | Exon 10-5′ |
| | | GUCUAUGU | | ATGTGTTTGGG | | | |
| | | GUUU | | | | | |
| gRNA1202 | TSBTx3835 | UCUCACCU | 3526 | TCTCACCTCTGC | 3530 | Y | Exon 16-3′ |
| | | CUGCAAGU | | AAGTATTGGGG | | | |
| | | AUUG | | | | | |
| gRNA1204 | TSBTx3837 | GCUUACAA | 3527 | GCTTACAATGAC | 3792 | N | Exon 11-3′ |
| | | UGACUGAG | | TGAGATCTTGG | | | |
| | | AUCU | | | | | |
| gRNA2067 | TSBTx3837 | UGCUUACA | 3528 | TGCTTACAATGA | 3793 | N | Exon 11-3′ |
| | | AUGACUGA | | CTGAGATCTTGG | | | |
| | | GAUCU | | | | | |

sg23/sgRNA_088 spacer sequence: CAGGAUCCGCACAGACUCCA (SEQ ID NO: 3794) (target gene: ALAS1).
The target sequence corresponding to sg23 is as follows, where the PAM sequence is in bold: CAGGATCCGCACAGACTCCAGGG (SEQ ID NO: 3795).
crRNA2/gRNA2002 spacer sequence: UCCCCGUUCUCGAAGUCGUG (SEQ ID NO: 3796).
Target site corresponding to crRNA2 (TSBTx4946): TCCCCGTTCTCGAAGTCGTGTGG (SEQ ID NO: 3797), where the PAM sequence is in bold.

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase, cytidine deaminase, or a dual deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain comprises an endonuclease or an exonuclease.

Disclosed herein are base editors comprising a polynucleotide programmable nucleotide binding domain comprising all or a portion (e.g., a functional portion) of a CRISPR protein (i.e., a base editor comprising as a domain all or a portion (e.g., a functional portion) of a CRISPR protein (e.g., a Cas protein), also referred to as a “CRISPR protein-derived domain” of the base editor). A CRISPR protein-derived domain incorporated into a base editor can be modified compared to a wild-type or natural version of the CRISPR protein. A CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2c1 (e.g., SEQ ID NO: 232), Cas12c/C2c3, Cas12d/CasY, Cas12e/CasX, Cas12g, Cas12h, Cas12i, and Cas12j/CasΦ, CARF, DinG, Turbo Cas9 (i.e., an SpCas9 with the amino acid alterations Q844R, V842L, F846Y, L847M, and I852F), homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof.

In some embodiments, a CRISPR protein-derived domain of a base editor can include all or a portion (e.g., a functional portion) of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 233.

In some embodiments, any of the Cas9 fusion proteins or complexes provided herein comprise one or more of a D10A, N497X, a R661X, a Q695X, and/or a Q926X mutation, or a corresponding mutation in any of the amino acid sequences provided herein, wherein X is any amino acid.

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. In some embodiments, any of the fusion proteins or complexes provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the napDNAbp is a circular permutant (e.g., SEQ ID NO: 238).

In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). For example, where a polynucleotide programmable nucleotide binding domain comprises a nickase domain derived from Cas9, the Cas9-derived nickase domain can include a D10A mutation and a histidine at position 840. In another example, a Cas9-derived nickase domain comprises an H840A mutation, while the amino acid residue at position 10 remains a D.

In some embodiments, a Cas9 nuclease has an inactive (e.g., an inactivated) DNA cleavage domain, that is, the Cas9 is a nickase, referred to as an “nCas9” protein (for “nickase” Cas9; SEQ ID No: 201). The Cas9 nickase may be a Cas9 protein that is capable of cleaving only one strand of a duplexed nucleic acid molecule (e.g., a duplexed DNA molecule). In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field and are within the scope of this disclosure.

Also provided herein are base editors comprising a polynucleotide programmable nucleotide binding domain which is catalytically dead (i.e., incapable of cleaving a target polynucleotide sequence). For example, in the case of a base editor comprising a Cas9 domain, the Cas9 can comprise both a D10A mutation and an H840A mutation. In further embodiments, a catalytically dead polynucleotide programmable nucleotide binding domain comprises a point mutation (e.g., D10A or H840A) as well as a deletion of all or a portion (e.g., a functional portion) of a nuclease domain. dCas9 domains are known in the art and described, for example, in Qi et al., “Repurposing CRISPR as an RNA-guided platform for sequence-specific control of gene expression.” Cell. 2013; 152 (5): 1173-83, the entire contents of which are incorporated herein by reference.

The term “protospacer adjacent motif (PAM)” or PAM-like motif refers to a 2-6 base pair DNA sequence immediately following the DNA sequence targeted by a nucleic acid programmable DNA binding protein. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR (N), TTTV, TYCV, TYCV, TATV, NNNNGATT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R. T. Walton et al., 2020, Science, 10.1126/science.aba8853 (2020), the entire contents of which are incorporated herein by reference.

Several PAM variants are described in Table 3 below.

TABLE 3
Cas9 proteins and corresponding PAM sequences.
N is A, C, T, or G; and Vis A, C, or G.

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-LRKIQK | NGTN |
| spCas9-LRVSQK | NGTN |
| spCas9-LRVSQL | NGTN |
| Cpf1 | 5′ (TTTV) |
| SpyMac | 5′-NAA-3′ |

In some embodiments, the PAM is NGC. In some embodiments, the NGC PAM is recognized by a Cas9 variant. In some embodiments, the Cas9 variant contains one or more amino acid substitutions selected from D1135V, G1218R, R1335Q, and T1337R (collectively termed VRQR) of spCas9 (SEQ ID No: 197), or a corresponding mutation in another Cas9. In some embodiments, the Cas9 variant contains one or more amino acid substitutions selected from D1135V, G1218R, R1335E, and T1337R (collectively termed VRER) of spCas9 (SEQ ID No: 197), or a corresponding mutation in another Cas9. In some embodiments, the Cas9 variant contains one or more amino acid substitutions selected from E782K, N968K, and R1015H (collectively termed KHH) of saCas9 (SEQ ID NO: 218).

In some cases, a Cas9 variant has specificity for the PAM 5′-NGC-3′. In some embodiments, a Cas9 variant includes one or more amino acid substitutions selected from D1135M, S1136Q, G1218K, E1219F, A1322R, D1332A, R1335E, and T1337K of spCas9 (SEQ ID No: 197), or a corresponding mutation in another Cas9. In some embodiments, a Cas9 variant includes one or more amino acid substitutions selected from D1135M, S1136Y, G1218K, E1219F, A1322R, D1332A, R1335E, and T1337K of spCas9 (SEQ ID No: 197), or a corresponding mutation in another Cas9. In some embodiments, the a Cas9 variant includes one or more amino acid substitutions selected from D1135L, S1136Y, G1218K, E1219F, A1322R, D1332A, R1335E, and T1337R of spCas9 (SEQ ID No: 197), or a corresponding mutation in another Cas9. In some embodiments, a Cas9 variant includes one or more amino acid substitutions selected from D1135M, S1136Y, G1218K, E1219F, A1322R, D1332A, R1335E, and T1337K of spCas9 (SEQ ID No: 197), or a corresponding mutation in another Cas9. In some embodiments, a Cas9 variant includes one or more amino acid substitutions selected from D1135L, S1136Y, G1218K, E1219F, A1283D, A1322R, D1332A, R1335E, and T1337K of spCas9 (SEQ ID No: 197), or a corresponding mutation in another Cas9. In some embodiments, a Cas9 variant includes one or more amino acid substitutions selected from A61R, L1111R, D1135L, S1136W, G1218K, E1219Q, N1317R, A1322R, R1333P, R1335Q, and T1337R of spCas9 (SEQ ID No: 197) (SpRY), or a corresponding mutation in another Cas9. In some embodiments, a Cas9 variant includes one or more amino acid substitutions selected from D1135L, S1136Q, G1218K, E1219F, E1250K, A1283D, A1322R, D1332A, R1335E, and T1337K of spCas9 (SEQ ID No: 197), or a corresponding mutation in another Cas9. In some embodiments, a Cas9 variant includes one or more amino acid substitutions selected from D1135M, S1136Y, G1218K, E1219F, E1250K, A1283D, A1322R, D1332A, R1335E, and T1337R of spCas9 (SEQ ID No: 197), or a corresponding mutation in another Cas9. In some embodiments, a Cas9 variant includes one or more amino acid substitutions selected from R765A, Q768A, D1135L, S1136Y, G1218K, A1283D, E1219F, A1322R, D1332A, R1335E, and T1337K of spCas9 (SEQ ID No: 197), or a corresponding mutation in another Cas9. In some embodiments, any of the Cas9 proteins provided herein, including an SpCas9 comprises any one, two, three, four, five, six, seven, eight, nine, or ten of the following amino acid substitutions in a corresponding residue: R765A, Q768A, W1126R, R1359W, E1250K, A1239T, A1239V, A1283D, R1335D, D1135L, D1135M, D1135R, D1135W, S1136H, S1136Q, S1136Y, G1218D, G1218K, G1218R, G1218E, G1218L, E1219F, E1219K, E1219N, A1322A, A1322R, A1322K, D1332A, R1335V, T1337K, T1337T, D1332A, D1135V and T1337R.

In some embodiments, a CRISPR protein-derived domain of a base editor comprises all or a portion (e.g., a functional portion) of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556 (7699), 57-63; Miller et al., “Continuous evolution of SpCas9 variants compatible with non-G PAMs” Nat. Biotechnol., 2020 April; 38 (4): 471-481; the entire contents of each are hereby incorporated by reference.

Fusion Proteins or Complexes Comprising a NapDNAbp and a Cytidine Deaminase and/or Adenosine Deaminase

Some aspects of the disclosure provide fusion proteins or complexes comprising a Cas9 domain or other nucleic acid programmable DNA binding protein (e.g., Cas12) and one or more cytidine deaminase, adenosine deaminase, or cytidine adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion proteins or complexes comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins or complexes of the present disclosure may comprise one or more additional features. For example, in some embodiments, the fusion protein or complex may comprise inhibitors, cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FLASH tags, V5 tags, and SBP-tags. Additional suitable sequences will be apparent to those of skill in the art. In some embodiments, the fusion protein or complex comprises one or more His tags.

Exemplary, yet nonlimiting, fusion proteins are described in International PCT Application Nos. PCT/US2017/045381, PCT/US2019/044935, and PCT/US2020/016288, each of which is incorporated herein by reference for its entirety.

Fusion Proteins or Complexes with Internal Insertions

Provided herein are fusion proteins or complexes comprising a heterologous polypeptide fused to a nucleic acid programmable nucleic acid binding protein, for example, a napDNAbp. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., cytidine or adenosine deaminase) or a functional fragment thereof. For example, a fusion protein can comprise a deaminase flanked by an N-terminal fragment and a C-terminal fragment of a Cas9 or Cas12 (e.g., Cas12b/C2c1), polypeptide.

The deaminase can be a circular permutant deaminase. In some embodiments, the deaminase is a circular permutant TadA, circularly permutated at amino acid residue 116, 136, or 65 as numbered in a TadA reference sequence.

The fusion protein or complexes can comprise more than one deaminase. The fusion protein or complex can comprise, for example, 1, 2, 3, 4, 5 or more deaminases. The deaminases in a fusion protein or complex can be adenosine deaminases, cytidine deaminases, or a combination thereof.

In some embodiments, the napDNAbp in the fusion protein or complex contains a Cas9 polypeptide or a fragment thereof. The Cas9 polypeptide can be a variant Cas9 polypeptide. The Cas9 polypeptide can be a circularly permuted Cas9 protein.

The heterologous polypeptide (e.g., deaminase) can be inserted in the napDNAbp (e.g., Cas9 or Cas12 (e.g., Cas12b/C2c1)) at a suitable location, for example, such that the napDNAbp retains its ability to bind the target polynucleotide and a guide nucleic acid. A deaminase (e.g., adenosine deaminase, cytidine deaminase, or adenosine deaminase and cytidine deaminase (dual deaminase)) can be inserted into a napDNAbp without compromising function of the deaminase (e.g., base editing activity) or the napDNAbp (e.g., ability to bind to target nucleic acid and guide nucleic acid).

In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in SEQ ID NO: 197. Cas9 polypeptide regions comprising a higher than average B-factor can include, for example, residues 792-872, 792-906, and 2-791 as numbered in SEQ ID NO: 197.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in SEQ ID NO: 197, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in SEQ ID NO: 197, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in SEQ ID NO: 197, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. Exemplary internal fusions base editors are provided in Table 4A below:

TABLE 4A
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant116 ins1067 | ISLAY29 |
| IBE031 | TadA- Circular Permutant 136 ins1248 | ISLAY31 |
| IBE032 | TadA- Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

A fusion protein can comprise a linker between the deaminase and the napDNAbp polypeptide. The linker can be a peptide or a non-peptide linker. For example, the linker can be an XTEN, (GGGS)ₙ (SEQ ID NO: 246), SGGSSGGS (SEQ ID NO: 330), (GGGGS)ₙ (SEQ ID NO: 247), (G)ₙ, (EAAAK)n (SEQ ID NO: 248), (GGS)ₙ, SGSETPGTSESATPES (SEQ ID NO: 249). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase but does not comprise a linker between the C-terminal Cas9 fragment and the deaminase. In some embodiments, the fusion protein comprises a linker between the C-terminal Cas9 fragment and the deaminase but does not comprise a linker between the N-terminal Cas9 fragment and the deaminase.

In some embodiments, the napDNAbp in the fusion protein or complex is a Cas12 polypeptide, e.g., Cas12b/C2c1, or a functional fragment thereof capable of associating with a nucleic acid (e.g., a gRNA) that guides the Cas12 to a specific nucleic acid sequence. The Cas12 polypeptide can be a variant Cas 12 polypeptide. In other embodiments, the N- or C-terminal fragments of the Cas12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS (SEQ ID NO: 250) or GSSGSETPGTSESATPESSG (SEQ ID NO: 251). In other embodiments, the linker is a rigid linker. In other embodiments of the above aspects, the linker is encoded by GGAGGCTCTGGAGGAAGC (SEQ ID NO: 252) or GGCTCTTCTGGATCTGAAACACCTGGCACAAGCGAGAGCGCCACCCCTGAGAGCTCTGGC (SEQ ID NO: 253).

In other embodiments, the fusion protein or complex contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence:

ATGGCCCCAAAGAAGAAGCGGAAGGTCGGTATCCACGGAGTCCCAGCAGCC (SEQ ID NO: 262). In other embodiments, the Cas12b polypeptide contains a mutation that silences the catalytic activity of a RuvC domain. In other embodiments, the Cas12b polypeptide contains D574A, D829A and/or D952A mutations.

In some embodiments, the fusion protein or complex comprises a napDNAbp domain (e.g., Cas12-derived domain) with an internally fused nucleobase editing domain (e.g., all or a portion (e.g., a functional portion) of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 4B below.

TABLE 4B
Insertion loci in Cas12b proteins

| | Insertion site | Inserted between aa |
|---|---|---|
| BhCas12b | | |
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | | |
| position 1 | 147 | PD |
| position 2 | 248 | GG |
| position 3 | 299 | PE |
| position 4 | 991 | GE |
| position 5 | 1031 | KM |
| AaCas12b | | |
| position 1 | 157 | PG |
| position 2 | 258 | VG |
| position 3 | 310 | DP |
| position 4 | 1008 | GE |
| position 5 | 1044 | GK |

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 263-308.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In an embodiment an adenosine deaminase domain of a base editor comprises all or a portion (e.g., a functional portion) of an ADAT comprising one or more mutations which permit the ADAT to deaminate a target A in DNA. For example, the base editor can comprise all or a portion (e.g., a functional portion) of an ADAT from Escherichia coli (EcTadA) comprising one or more of the following mutations: D108N, A106V, D147Y, E155V, L84F, H123Y, 1156F, or a corresponding mutation in another adenosine deaminase. Exemplary ADAT homolog polypeptide sequences are provided in the Sequence Listing as SEQ ID NOs: 1 and 309-315.

The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein.

It should be appreciated that any of the mutations provided herein (e.g., based on a TadA reference sequence, such as TadA*7.10 (SEQ ID NO: 1)) can be introduced into other adenosine deaminases, such as E. coli TadA (ecTadA), S. aureus TadA (saTadA), or other adenosine deaminases (e.g., bacterial adenosine deaminases). In some embodiments, the TadA reference sequence is TadA*7.10 (SEQ ID NO: 1). It would be apparent to the skilled artisan that additional deaminases may similarly be aligned to identify homologous amino acid residues that can be mutated as provided herein. Thus, any of the mutations identified in a TadA reference sequence can be made in other adenosine deaminases (e.g., ecTada) that have homologous amino acid residues. It should also be appreciated that any of the mutations provided herein can be made individually or in any combination in a TadA reference sequence or another adenosine deaminase.

In some embodiments, the adenosine deaminase comprises an alteration or set of alterations selected from those listed in Tables 5A-5G below:

TABLE 5A
Adenosine Deaminase Variants. Residue positions in the E. coli TadA variant (TadA*) are indicated.

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| TadA*0.1 | M | R | H | N | P | | R | N | L | S | A | D | H | G | A | S | D | R | E | I | K | K |
| TadA*0.2 | M | R | H | N | P | | R | N | L | S | A | D | H | G | A | S | D | R | E | I | K | K |
| TadA*1.1 | W | R | H | N | P | | R | IN | L | S | A | N | H | G | A | S | D | R | E | I | K | K |
| TadA*1.2 | M | R | H | N | P | | R | N | L | S | V | N | H | G | A | S | D | R | E | I | K | K |
| TadA*2.1 | M | R | H | N | P | | R | N | L | S | V | N | H | G | \|A | S | Y | R | V | I | K | K |
| TadA*2.2 | M | R | H | N | P | | R | N | L | S | V | IN | H | G | A | S | Y | R | V | I | K | K |
| TadA*2.3 | M | R | H | N | P | | R | N | L | S | V | N | H | G | JA | S | Y | R | V | I | K | K |
| TadA*2.4 | M | R | H | N | P | | R | N | L | S | V | N | H | G | A | S | Y | R | V | I | K | K |
| TadA*2.5 | M | R | H | N | P | | R | N | L | S | V | N | H | G | A | S | Y | R | V | I | K | K |
| TadA*2.6 | M | R | H | N | P | | R | N | L | S | V | N | H | G | A | S | Y | R | V | I | K | K |
| TadA*2.7 | W | R | H | N | IP | | R | N | L | S | V | N | H | G | A | S | Y | R | V | I | K | K |
| TadA*2.8 | M | R | H | N | P | | R | N | L | S | V | N | H | G | A | S | Y | R | V | I | K | K |
| TadA*2.9 | M | R | H | N | P | | R | N | L | S | V | N | H | G | A | S | Y | R | V | I | K | K |
| TadA*2.10 | M | R | H | N | P | | R | N | L | S | V | N | H | G | A | S | Y | R | V | I | K | K |
| TadA*2.11 | M | R | H | N | P | | R | N | L | S | V | N | H | G | A | S | Y | R | V | I | K | K |
| TadA*2.12 | M | R | H | N | P | | R | N | L | S | V | N | H | G | A | S | Y | R | V | I | K | K |
| TadA*3.1 | W | R | H | N | P | | R | N | F | S | V | N | Y | G | A | S | Y | R | V | F | K | K |
| TadA*3.2 | M | R | H | N | P | | R | N | F | S | V | N | Y | G | A | S | Y | R | V | F | K | K |
| TadA*3.3 | M | R | H | N | P | | R | N | F | S | V | N | Y | G | A | S | Y | R | V | F | K | K |
| TadA*3.4 | W | R | H | N | P | | R | N | F | S | V | N | Y | G | A | S | Y | R | V | F | K | K |
| TadA*3.5 | M | R | H | N | P | | R | IN | F | S | V | N | Y | G | A | S | Y | R | V | F | K | K |
| TadA*3.6 | M | R | H | N | P | | R | N | F | S | V | N | Y | G | A | S | Y | R | V | F | K | K |
| TadA*3.7 | M | R | H | N | P | | R | N | F | S | V | N | Y | G | A | S | Y | R | V | F | K | K |
| TadA*3.8 | M | R | H | N | P | | R | N | F | S | V | N | Y | G | A | S | Y | R | V | F | K | K |
| TadA*4.1 | W | R | H | N | P | | R | N | L | S | V | N | H | G | N | S | Y | R | V | I | K | K |
| TadA*4.2 | M | G | H | N | P | | R | N | L | S | V | N | H | G | N | S | Y | R | V | I | K | K |
| TadA*4.3 | M | R | H | N | P | | R | N | F | S | V | N | Y | G | N | S | Y | R | V | F | K | K |
| TadA*5.1 | M | R | L | N | P | | L | N | F | S | V | N | Y | G | A | C | Y | R | V | F | N | K |
| TadA*5.2 | M | R | H | S | P | | R | N | F | S | V | N | Y | G | A | S | Y | R | V | F | K | T |
| TadA*5.3 | M | R | L | N | P | | L | N | I | S | V | N | Y | G | A | C | Y | R | V | F | N | K |
| TadA*5.4 | W | R | H | S | P | | R | N | F | S | V | N | Y | G | A | S | Y | R | V | F | K | T |
| TadA*5.5 | M | R | L | N | P | | L | N | F | S | V | N | Y | G | A | C | Y | R | V | F | N | K |
| TadA*5.6 | M | R | L | N | P | | L | N | F | S | V | N | Y | G | A | C | Y | R | V | F | N | K |
| TadA*5.7 | W | R | L | N | P | | L | N | F | S | V | N | Y | G | A | C | Y | R | V | F | N | K |
| TadA*5.8 | M | R | L | N | P | | L | N | F | S | V | N | Y | G | A | C | Y | R | V | F | N | K |
| TadA*5.9 | M | R | L | N | P | | L | N | F | S | V | N | Y | G | A | C | Y | R | V | F | N | K |
| TadA*5.10 | M | R | L | N | P | | L | IN | F | S | V | N | Y | G | A | C | Y | R | V | F | N | K |
| TadA*5.11 | M | R | L | N | P | | L | N | F | S | V | N | Y | G | A | C | Y | R | V | F | N | K |
| TadA*5.12 | W | R | L | N | P | | L | IN | F | S | V | N | Y | G | A | C | Y | R | V | F | N | K |
| TadA*5.13 | W | R | H | N | P | | L | D | \|F | S | V | N | Y | A | A | S | Y | R | V | F | K | K |
| TadA*5.14 | W | R | H | N | S | | L | IN | F | C | V | N | Y | G | A | S | Y | R | V | F | K | K |
| TadA*6.1 | W | R | H | N | S | | L | N | F | S | V | N | Y | G | N | S | Y | R | V | F | K | K |
| TadA*6.2 | M | R | H | N | T | V | L | N | F | S | V | N | Y | G | N | S | Y | R | V | F | N | K |
| TadA*6.3 | M | R | L | N | S | | L | N | F | S | V | N | Y | G | A | C | Y | R | V | F | N | K |
| TadA*6.4 | M | R | L | N | S | | L | N | F | S | V | N | Y | G | N | C | Y | R | V | F | N | K |
| TadA*6.5 | W | R | T | N | T | V | L | N | F | S | V | N | Y | G | A | C | Y | R | V | F | N | K |
| TadA*6.6 | M | R | L | N | T | V | L | N | F | S | V | N | Y | G | N | C | Y | R | V | F | N | K |
| TadA*7.1 | W | R | L | N | A | | L | N | F | S | V | N | Y | G | A | C | Y | R | V | F | N | K |
| TadA*7.2 | W | R | L | N | A | | L | N | F | S | V | N | Y | G | N | C | Y | R | V | F | N | K |
| TadA*7.3 | T | R | L | N | A | | L | N | F | S | V | N | Y | G | A | C | Y | R | V | F | N | K |
| TadA*7.4 | R | R | L | N | A | | L | N | F | S | V | N | Y | G | A | C | Y | R | V | F | N | K |
| TadA*7.5 | W | R | L | N | A | | L | N | F | S | V | N | Y | G | A | C | Y | H | V | F | N | K |
| TadA*7.6 | W | R | L | N | A | | L | N | I | S | V | N | Y | G | A | C | Y | P | V | F | N | K |
| TadA*7.7 | | R | L | N | A | | L | N | F | S | V | N | Y | G | A | C | Y | P | V | F | N | K |
| TadA*7.8 | L | R | L | N | A | | L | N | F | S | V | N | Y | G | N | C | Y | R | V | IF | N | K |
| TadA*7.9 | T | R | L | N | A | | L | N | F | S | V | N | Y | G | N | C | Y | P | V | \|F | N | K |
| TadA*7.10 | R | R | L | N | A | | L | N | \|F | S | V | N | Y | G | A | C | Y | P | V | F | N | K |

TABLE 5B
TadA*8 Adenosine Deaminase Variants. Residue positions in the E. coli TadA variant (TadA*) are indicated. Alterations are referenced to TadA*7.10 (first row).

| | 2 | 3 | 4 | 5 | 7 | 8 | 8 | 10 | 10 | 12 | 14 | 14 | 15 | 15 | 15 | 15 | 15 | 166 |
|---|---|---|---|---|---|---|---|---|---|---|---|---|---|---|---|---|---|---|
| | 3 | 6 | 8 | 1 | 6 | 2 | 4 | 6 | 8 | 3 | 6 | 7 | 2 | 4 | 5 | 6 | 7 | |
| TadA*7.10 | R | L | A | L | I | V | F | V | N | Y | C | Y | P | Q | V | F | N | T |
| TadA*8.1 | | | | | | | | | | | | T | | | | | | |
| TadA*8.2 | | | | | | | | | | | | R | | | | | | |
| TadA*8.3 | | | | | | | | | | | | | | S | | | | |
| TadA*8.4 | | | | | | | | | | H | | | | | | | | |
| TadA*8.5 | | | | | | S | | | | | | | | | | | | |
| TadA*8.6 | | | | | | | | | | | | | | | | | | R |
| TadA*8.7 | | | | | | | | | | | | | | R | | | | |
| TadA*8.8 | | | | | | | | | | H | | R | | R | | | | |
| TadA*8.9 | | | | | Y | | | | | | | R | | R | | | | |
| TadA*8.10 | | | | | | | | | | | | R | | R | | | | R |
| TadA*8.11 | | | | | | | | | | | | T | | R | | | | |
| TadA*8.12 | | | | | | | | | | | | T | | S | | | | |
| TadA*8.13 | | | | | Y | | | | | H | | R | | R | | | | |
| TadA*8.14 | | | | | Y | S | | | | | | | | | | | | |
| TadA*8.15 | | | | | | S | | | | | | R | | | | | | |
| TadA*8.16 | | | | | | S | | | | H | | R | | | | | | |
| TadA*8.17 | | | | | | S | | | | | | | | R | | | | |
| TadA*8.18 | | | | | | S | | | | H | | | | R | | | | |
| TadA*8.19 | | | | | | S | | | | H | | R | | R | | | | |
| TadA*8.20 | | | | | Y | S | | | | H | | R | | R | | | | |
| TadA*8.21 | | | | | | | | | | | | R | | S | | | | |
| TadA*8.22 | | | | | | S | | | | | | | | S | | | | |
| TadA*8.23 | | | | | | S | | | | H | | | | | | | | |
| TadA*8.24 | | | | | | S | | | | H | | T | | | | | | |

TABLE 5C
TadA*9 Adenosine Deaminase Variants. Alterations are referenced
to TadA*7.10. Additional details of TadA*9 adenosine
deaminases are described in International PCT Application
No. PCT/US2020/049975, which is incorporated herein by
reference in its entirety for all purposes.

| TadA*9 Description | Alterations |
|---|---|
| TadA*9.1 | E25F, V82S, Y123H, T133K, Y147R, Q154R |
| TadA*9.2 | E25F, V82S, Y123H, Y147R, Q154R |
| TadA*9.3 | V82S, Y123H, P124W, Y147R, Q154R |
| TadA*9.4 | L51W, V82S, Y123H, C146R, Y147R, Q154R |
| TadA*9.5 | P54C, V82S, Y123H, Y147R, Q154R |
| TadA*9.6 | Y73S, V82S, Y123H, Y147R, Q154R |
| TadA*9.7 | N38G, V82T, Y123H, Y147R, Q154R |
| TadA*9.8 | R23H, V82S, Y123H, Y147R, Q154R |
| TadA*9.9 | R21N, V82S, Y123H, Y147R, Q154R |
| TadA*9.10 | V82S, Y123H, Y147R, Q154R, A158K |
| TadA*9.11 | N72K, V82S, Y123H, D139L, Y147R, Q154R, |
| TadA*9.12 | E25F, V82S, Y123H, D139M, Y147R, Q154R |
| TadA*9.13 | M70V, V82S, M94V, Y123H, Y147R, Q154R |
| TadA*9.14 | Q71M, V82S, Y123H, Y147R, Q154R |
| TadA*9.15 | E25F, V82S, Y123H, T133K, Y147R, Q154R |
| TadA*9.16 | E25F, V82S, Y123H, Y147R, Q154R |
| TadA*9.17 | V82S, Y123H, P124W, Y147R, Q154R |
| TadA*9.18 | L51W, V82S, Y123H, C146R, Y147R, Q154R |
| TadA*9.19 | P54C, V82S, Y123H, Y147R, Q154R |
| TadA*9.2 | Y73S, V82S, Y123H, Y147R, Q154R |
| TadA*9.21 | N38G, V82T, Y123H, Y147R, Q154R |
| TadA*9.22 | R23H, V82S, Y123H, Y147R, Q154R |
| TadA*9.23 | R21N, V82S, Y123H, Y147R, Q154R |
| TadA*9.24 | V82S, Y123H, Y147R, Q154R, A158K |
| TadA*9.25 | N72K, V82S, Y123H, D139L, Y147R, Q154R, |
| TadA*9.26 | E25F, V82S, Y123H, D139M, Y147R, Q154R |
| TadA*9.27 | M70V, V82S, M94V, Y123H, Y147R, Q154R |
| TadA*9.28 | Q71M, V82S, Y123H, Y147R, Q154R |
| TadA*9.29 | E25F, I76Y, V82S, Y123H, Y147R, Q154R |
| TadA*9.30 | I76Y, V82T, Y123H, Y147R, Q154R |
| TadA*9.31 | N38G, I76Y, V82S, Y123H, Y147R, Q154R |
| TadA*9.32 | N38G, I76Y, V82T, Y123H, Y147R, Q154R |
| TadA*9.33 | R23H, I76Y, V82S, Y123H, Y147R, Q154R |
| TadA*9.34 | P54C, I76Y, V82S, Y123H, Y147R, Q154R |
| TadA*9.35 | R21N, I76Y, V82S, Y123H, Y147R, Q154R |
| TadA*9.36 | I76Y, V82S, Y123H, D138M, Y147R, Q154R |
| TadA*9.37 | Y72S, I76Y, V82S, Y123H, Y147R, Q154R |
| TadA*9.38 | E25F, I76Y, V82S, Y123H, Y147R, Q154R |
| TadA*9.39 | I76Y, V82T, Y123H, Y147R, Q154R |
| TadA*9.40 | N38G, I76Y, V82S, Y123H, Y147R, Q154R |
| TadA*9.41 | N38G, I76Y, V82T, Y123H, Y147R, Q154R |
| TadA*9.42 | R23H, I76Y, V82S, Y123H, Y147R, Q154R |
| TadA*9.43 | P54C, I76Y, V82S, Y123H, Y147R, Q154R |
| TadA*9.44 | R21N, I76Y, V82S, Y123H, Y147R, Q154R |
| TadA*9.45 | I76Y, V82S, Y123H, D138M, Y147R, Q154R |
| TadA*9.46 | Y72S, I76Y, V82S, Y123H, Y147R, Q154R |
| TadA*9.47 | N72K, V82S, Y123H, Y147R, Q154R |
| TadA*9.48 | Q71M, V82S, Y123H, Y147R, Q154R |
| TadA*9.49 | M70V, V82S, M94V, Y123H, Y147R, Q154R |
| TadA*9.50 | V82S, Y123H, T133K, Y147R, Q154R |
| TadA*9.51 | V82S, Y123H, T133K, Y147R, Q154R, A158K |
| TadA*9.52 | M70V, Q71M, N72K, V82S, Y123H, Y147R, |
| | Q154R |
| TadA*9.53 | N72K, V82S, Y123H, Y147R, Q154R |
| TadA*9.54 | Q71M, V82S, Y123H, Y147R, Q154R |
| TadA*9.55 | M70V, V82S, M94V, Y123H, Y147R, Q154R |
| TadA*9.56 | V82S, Y123H, T133K, Y147R, Q154R |
| TadA*9.57 | V82S, Y123H, T133K, Y147R, Q154R, A158K |
| TadA*9.58 | M70V, Q71M, N72K, V82S, Y123H, Y147R, |
| | Q154R |

In some embodiments, the adenosine deaminase comprises a TadA*8.20 adenosine deaminase variant further comprising an F149Y amino acid alteration. In some embodiments, the adenosine deaminase comprises a TadA*8.20 adenosine deaminase variant further comprising the amino acid alterations R147D, F149Y, T166I, and D167N (TadA*8.10+). In some embodiments, the adenosine deaminase comprises a TadA*8.20 adenosine deaminase variant further comprising the amino acid alterations S82T and F149Y (TadA*9v1). In some embodiments, the adenosine deaminase comprises a TadA*8.20 adenosine deaminase variant further comprising the amino acid alterations Y147D, F149Y, T166I, D167N and S82T (TadA*9v2).

In some embodiments, the adenosine deaminase comprises one or more of M1I, M1S, S2A, S2E, S2H, S2R, S2L, E3L, V4D, V4E, V4M, V4K, V4S, V4T, V4A, E5K, F6S, F6G, F6H, F6Y, F6I, F6E, S7K, H8E, H8Y, H8H, H8Q, H8E, H8G, H8S, E9Y, E9K, E9V, E9E, Y10F, Y10W, Y10Y, M12S, M12L, M12R, M12W, R13H, R13I, R13Y, R13R, R13G, R13S, H14N, A15D, A15V, A15L, A15H, T17T, T17A, T17W, T17L, T17F, T17R, T17S, L18A, L18E, L18N, L18L, L18S, A19N, A19H, A19K, A19A, A19D, A19G, A19M, R21N, K20K, K20A, K20R, K20E, K20G, K20C, K20Q R21A, R21R, R21N, R21Y, R21C G22P, A22W, A22R, W23D, R23H, W23G, W23Q, W23L, W23R, W23H W23D W23M, W23W, W23I, D24E, D24G, D24W, D24D, D24R, E25F, E25M, E25D, E25A, E25G, E25R, E25E, E25H E25V, E25S, E25Y, R26D, R26E, R26G, R26N, R26Q, R26C, R26L, R26K, R26W, R26C, R26P, R26R, R26A, R26H, E27E, E27Q, E27H, E27C, E27G, E27K, E27S, E27P, E27R, E27L, E27V, E27D, V28V, V28A, V28C, V28G, V28P, V28S, V28T, P29V, P29P, P29A, P29G, P29K, P29L, V30V, V30I, V30L, V30F, V30G, V30A, V30M, L34S, L34V, L34L, L34M, L34W, L34G, H36E, H36V, L36H, H36L, H36N, N37N, N37H, N37R, N37T, N37S, N38G, N38R, N38N, N38E, V40I, W45A, W45W, W45R, W45L, W45N, N46N, N46M, N46P, N46G, N46L, N46R, N46V, R46W, R46F, R46Q, R46M, R47A, R47Q, R47F, R47K, R47P, R47W, R47M, R47R, R47G, R47S, R47V, R47H, P48T, P48L, P48A, P48I, P48S, P48R, P48K, P48D, P48E, P48H, P48G, P48P, P48N, 149G, 149H, 149V, 149F, 149H, I491, 149M, 149N, 149K, 149Q, I49T, G50L, G50S, G50R, G50G, R51H, R51L, R51N, L51W, R51Y, R51G, R51V, R51R, H52D, H52Y, H52I, H52H, D53D, D53E, D53G, D53P, P54C, P54T, P54P, P54E, A55H, T55A, T55I, T55V, T55G, T55T, A56A, A56H, A56W, A56E, A56S, H57P, H57A, H57H, H57N, A58G, A58E, A58A, A58R, E59A, E59G, E59I, E59Q, E59W, E59E, E59T, E59H, E59P, M61A, M61I, M61L, M61V, M61P, M61G, M61I, L63S, L63V, L63T, L63R, L63H, L63A, R64A, R64Q, R64R, R64D, Q65V, Q65H, Q65G, Q65P, Q65F, Q65Q, Q65R, G66V, G66E, G66T, G66G, G66C, G67G, G67W, G67I, G67A, G67D, G67L, G67V, L68Q, L68M, L68V, L68H, L68L, L68G, V69A, V69M, V69V, M70V, M70L, E70A, M70A, M70M, M70E, M70T, M70v, Q71M, Q71N, Q71L, Q71R, Q71Q, Q71I, N72A, N72K, N72S, N72D, N72Y, N72N, N72H, N72G, N72M, Y73G, Y73I, Y73K, Y73R, Y73S, Y73Y, Y73H, Y73A, R74A, R74Q, R74G, R74K, R74L, R74N, R74G, R74K, R74R, 176H, I76R, 176W, 176Y, 176V, 176Q, 176L, 176D, 176F, 176I, 176N, I76T, I76Y, D77G, D77D, D77A, D77Q, A78Y, A78T, A78G, A78A, A781, T79M, T79R, T79L, T79T, L80M, L80Y, L80I, L80V, L80L, Y81D, Y81V, Y81Y, Y81M, V82A, V82S, V82G, V82T, V82V, V82Q, V82Y, T83L, T83F, T83T, T83N, L84E, L84F, L84Y, L84I, L84L, L84M, L84A, L84T, L84S, E85K, E85G, E85P, E85S, E85E, E85F, E85V, E85R, P86T, P86C, P86P, P86L, P86N, P86K, P86H, C87M, C87I, C87S, C87N, C87P, S87C, S87L, S87V, V88A, V88M, V88V, V88T, V88E, V88D, V88S, C90S, C90P, C90A, C90T, C90M, A91A, A91G, A91S, A91V, A91T, A91C, A91L, G92T, G92M, G92A, G92Y, G92G, A93I, A93C, A93M, A93V, A93A, M94M, M94T, M94A, M94V, M94L, M94I, M94H, 195S, 195G, 195L, 195H, 195V, H96A, H96L, H96R, H96S, H96H, H96N, H96E, S97C, S97G, S97I, S97M, S97R, S97S, S97P, R98K, R981, R98N, R98Q, R98G, R98H, R98C, R98L, R98R, G100R, G100V, G100K, G100A, G100S, G100M, G100I, R101V, R101R, R101S, R101C, V102A, V102F, V102I, V102V, D103A, V103A, V103G, V103F, V103V, F104G, D104N, F104V, F104I, F104L, F104A, F104F, F104R, G105V, G105W, G105G, G105M, G105A, A106T, V106Q, V106F, V106W, V106M, A106A, A106Q, A106F, A106G, A106W, A106M, A106V, A106R, A106L, A106S, A106B, A106I, R107C, R107G, R107P, R107K, R107A, R107N, R107W, R107H, R107S, R107R, R107F, D108N, D108F, D108G, D108V, D108A, D108Y, D108H, D108I, D108K, D108L, D108M, D108Q, N108Q, N108F, N108W, N108M, N108K, D108K, D108F, D108M, D108Q, D108R, D108W, D108S, D108E, D108T, D108R, D108D, A109H, A109K, A109R, A109S, A109T, A109V, A109A, A109D, K110G, K110H, K110I, K110R, K110T, K110K, K110A, K1101, T111A, T111G, T111H, T111R, T111T, T111K, G112A, G112G, G112H, G112T, G112R, A113N, A114G, A114H, A114V, A114C, A114S, A114A, G115S, G115G, G115M, G115L, G115A, G115F, L117M, L117L, L117W, L117A, L117S, L117N, L117V, M118D, M118G, M118K, M118N, M118V, M118M, M118L, M118R, D119L, D119N, D119S, D119V, D119D, V120H, V120L, V120V, V120T, V120A, V120E, V120G, V120D, L121D, L121M, L121N, L121K, L121L, H122H, H122N, H122P, H122R, H122S, H122Y, H122G, H122T, H122L, H123C, H123G, H123P, H123V, H123Y, Y123H, H123Y, H123H, P124P, P124H, P124A, P124Y, P124D, P124G, P124I, P124L, P124W, G125H, G125I, G125A, G125M, G125K, G125G, G125P, M126D, M126H, M126K, M126I, M126N, M1260, M126S, M126Y, M126M, M126G, N127H, N127S, N127D, N127K, N127R, N127N, N127I, N127P, N127M, H128R, H128N, H128L, H128H, R129H, R129Q, R129V, R129I, R129E, R129V, R129R, R129M, R129P, V130R, V130V, V130E, V130D, E131E, E131I, E131V, E131K, I1321, 1132F, 1132T, 1132L, 1132V, 1132E, T133V, T133E, T133G, T133K, T133T, T133A, T133H, T133F, T133I, E134A, E134E, E134G, E134I, E134H, E134K, E134T, G135G, G135V, G135I, G135P, G135E, 1136G, I136L, 1136T, 11361, 1137A, 1137D, 1137E, L137M, 1137S, L137L, L1371, A138D, A138E, A138G, S138A, A138N, A138S, A138T, A138V, A138Y, A138A, A138M, A138L, D139E, D1391, D139C, D139L, D139M, D139D, D139G, D139H, D139A, E140A, E140C, E140L, E140R, E140K, E140E, E140D, C141S, C141A, C141C, C141V, C141E, A142N, A142D, A142G, A142A, A142L, A142S, A142T, A142N, A142S, A142V, A142E, A142C, A143D, A143E, A143G, A143D, A143G, A143E, A143L, A143W, A143M, A143S, A143Q, A143R, A143A, A143I, L144S, L144L, L144T, L144A, L145A, L145F, L145G, L145D, L145L, L145C, L145E, L145s, C146R, S146A, S146C, S146D, S146F, S146R, S146T, S146D, S146G, S146S, S146L, D147D, D147L, D147F, D147G, D147Y, Y147T, Y147R, Y147D, D147R, D147Y, D147A, D147T, D147H, D147F, D147U, D147V, D147I, D147C, F148L, F148F, F148R, F148Y, F148A, F148T, F149C, F149M, F149R, F149Y, F149N, F149F, F149A, F149T, F149V R150R, R150M, R150D, R150F, M151F, M151P, M151R, M151V, M151M, M151E, R152C, R152F, R152H, R152P, R152R, R152P, R152Q, R152M, R1520, R153C, R153Q, R153R, R153V, R153E, R153A, R153P, Q154E, Q154H, Q154M, Q154R, Q154L, Q154S, Q154V, Q154Q, Q154F, Q154I, Q154A, Q154K, E155F, E155G, E155I, E155K, E155P, E155V, E155D, E155E, E155L, E155Q, I156V, 1156A, 1156I, 1156L, 1156F, 1156D, 1156K, 1156N, 1156R, 1156Y, E157A, E157F, E1571, E157P, E157T, E157V, N157K, K157N, K157V, K157P, K157I, K157F, K157F, K157T, K157A, K157S, K157R, A158Q, A158K, A158V, A158A, A158D, A158S, A158T, A158N, Q159S, Q159Q, Q159A, Q159F, Q159K, Q159L, Q159N, K160A, K160S, K160E, K160K, K160N, K160F, K160Q, K161T, K161K, K161R, K161I, K161A, K161N, K161Q, K161S, K161T, A162D, A162Q, R162H, R162P, A162S, A162A, A162N, A162M, A162K, Q163G, Q163S, Q163Q, Q163A, Q163H, Q163N, Q163R, S164F, S164S, S164Q, S164I, S164R, S164Y, S165S, S165P, S165Q, S165A, S165D, S165I, S165T, S165Y, T166T, T166Q, T166E, T166S, T166D, T166K, T166I, T166N, T166P, T166R, D167S D167D, D1671, D167G, D167T, D167A and/or D167N mutation in a TadA reference sequence (e.g., TadA*7.10, ecTadA, or TadA8e), and any alternative mutation at the corresponding position, or one or more corresponding mutations in another adenosine deaminase. Additional mutations are described in U.S. Patent Application Publication No. 2022/0307003 A1 U.S. Pat. No. 11,155,803, and International Patent Application Publications No. WO 2023/288304 A2, PCT/CN2022/143408, WO 2018/027078 A1, WO 2021/158921 A1 and WO 2023/034959 A2, the disclosures of which are incorporated herein by reference in their entirety for all purposes.

In various embodiments, an adenosine deaminase of the disclosure lacks an N-terminal methionine.

In some embodiments, the disclosure provides TadA variants comprising an alteration at an amino acid selected from one or more of L36, 176, V82, Y147, Q154, and N157 comapred to TadA*7.10. In some embodiments, the disclosure provides TadA variants comprising one or more of the following alterations relative to TadA*7.10: L36H, 176Y, V82T, Y147T, Q154S, and N157K. In some embodiments, the disclosure provides TadA variants comprising the following alterations relative to TadA*7.10: L36H, 176Y, V82T, Y147T, Q154S, and N157K. In some embodiments, the disclosure provides TadA variants comprising the following alterations relative to TadA*7.10: F84Y, A109L, A109V, A109I, A109F, A109S, A109T, A109N, V155S, V155T, V155N, F156Y, F156W, F156R, F156N, and F156Q. In some embodiments, the disclosure provides TadA variants comprising the following alterations relative to TadA*7.10: E3N, E3K, E3G, F6A, H14D, L18A, W23I, W23R, P29T, P29Y, P29Q, V35Q, L36S, N38D, G42M, N46Y, P48A, G50A, H52L, A62V, L63R, L63F, Q65R, G67N, L68V, M701, N72Y, T79H, Y81V, V82S, M94R, G100V, V102E, V102S, R107A, A114C, G115E, M118L, D119L, H122T, P124H, P124K, P124Q, H128R, V130F, 1132K, I132T, E140L, A142N, A142S, L144Q, L145R, L145N, Y147A, F149A, R152P, F156N, and K160E.

In some embodiments, the disclosure provides TadA variants comprising a V82T, Y147T, and/or a Q154S mutation. In some embodiments, the disclosure provides TadA variants comprising a V82T, Y147T, and/or a Q154S mutation. In some embodiments, the disclosure provides TadA*8.8 further comprising a V82T mutation. In some embodiments, the disclosure provides TadA*8.8 further comprising a V82T, a Y147T, and a Q154S mutation. In some embodiments, the disclosure provides TadA*8.17 further comprising a V82T mutation. In some embodiments, the disclosure provides TadA*8.17 further comprising a V82T, a Y147T, and a Q154S mutation. In some embodiments, the disclosure provides TadA*8.20 further comprising a V82T mutation. In some embodiments, the disclosure provides TadA*8.20 further comprising a V82T, a Y147T, and a Q154S mutation.

In embodiments, a variant of TadA*7.10 comprises one or more alterations selected from any of those alterations provided herein.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, the TadA*8 is a variant as shown in Table 5D. Table 5D shows certain amino acid position numbers in the TadA amino acid sequence and the amino acids present in those positions in the TadA-7.10 adenosine deaminase. Table 5D also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e. In one embodiment, an adenosine deaminase is a TadA*8 that comprises or consists essentially of SEQ ID NO: 316 or a fragment thereof having adenosine deaminase activity.

TABLE 5D
TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA- | R | V | A | T | D | H | Y | F | T | D |
| | 7.10 | | | | | | | | | | |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 5E. Table 5E shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 5E
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

TABLE 5F
TadA Variants

| Description | | | | | | | | | | |
|---|---|---|---|---|---|---|---|---|---|---|
| Amino Acid No. | 23 | 36 | 48 | 51 | 76 | 82 | 84 | 106 | 108 | 123 |
| TadA (wt) | W | H | P | R | I | V | L | A | D | H |
| TadA*7.10 | R | L | A | L | I | V | F | V | N | Y |
| TadA*8.8 | | | | | | | | | | H |
| TadA*8.13 | | | | | Y | | | | | H |
| TadA*8.17 | | | | | | S | | | | |
| TadA*8.20 | | | | | Y | S | | | | H |
| TadA*8.8 + V82T | | | | | | T | | | | H |
| TadA*8.8 + | | | | | | T | | | | H |
| V82T + Y147T + | | | | | | | | | | |
| Q154S | | | | | | | | | | |
| TadA*8.17 + V82T | | | | | | T | | | | |
| TadA*8.17 + | | | | | | T | | | | |
| V82T + Y147T + | | | | | | | | | | |
| Q154S | | | | | | | | | | |
| TadA*8.20 + V82T | | | | | Y | T | | | | H |
| TadA*8.20 + | | | | | Y | T | | | | H |
| V82T + Y147T + | | | | | | | | | | |
| Q154S | | | | | | | | | | |

TadA Variants

| Official Name | | | | | | | | |
|---|---|---|---|---|---|---|---|---|
| Amino Acid No. | 146 | 147 | 152 | 154 | 155 | 156 | 157 | 166 |
| TadA (wt) | S | D | R | Q | E | I | K | T |
| TadA*7.10 | C | Y | P | O | V | F | N | T |
| TadA*8.8 | | R | | R | | | | |
| TadA*8.13 | | R | | R | | | | |
| TadA*8.17 | | | | R | | | | |
| TadA*8.20 | | R | | R | | | | |
| TadA*8.8 + V82T | | R | | R | | | | |
| TadA*8.8 + | | T | | S | | | | |
| V82T + Y147T + | | | | | | | | |
| Q154S | | | | | | | | |
| TadA*8.17 + V82T | | | | R | | | | |
| TadA*8.17 + | | T | | S | | | | |
| V82T + Y147T + | | | | | | | | |
| Q154S | | | | | | | | |
| TadA*8.20 + V82T | | R | | R | | | | |
| TadA*8.20 + | | T | | S | | | | |
| V82T + Y147T + | | | | | | | | |
| Q154S | | | | | | | | |

TABLE 5G
TadA Variants

| Official | Alternative | | | | | | | | | | | | | | | | |
|---|---|---|---|---|---|---|---|---|---|---|---|---|---|---|---|---|---|
| Name | Name | 76 | 82 | 84 | 109 | 111 | 119 | 122 | 123 | 146 | 147 | 149 | 154 | 155 | 156 | 166 | 167 |
| TadA | | I | V | L | A | T | D | H | H | S | D | F | Q | E | I | T | D |
| (wt) | | | | | | | | | | | | | | | | | |
| ABE7.10 | | I | V | F | A | T | D | H | Y | C | Y | F | Q | V | F | T | D |
| 8.8 + | | | T | | | | | | H | | R | | R | | | | |
| V82T | | | | | | | | | | | | | | | | | |
| 8.8 + | | | T | | | | | | H | | T | | S | | | | |
| V82T + | | | | | | | | | | | | | | | | | |
| Y147T + | | | | | | | | | | | | | | | | | |
| Q154S | | | | | | | | | | | | | | | | | |
| 8.17 + | | | T | | | | | | | | | | R | | | | |
| V82T | | | | | | | | | | | | | | | | | |
| 8.17 + | | | T | | | | | | | | T | | S | | | | |
| V82T + | | | | | | | | | | | | | | | | | |
| Y147T + | | | | | | | | | | | | | | | | | |
| Q154S | | | | | | | | | | | | | | | | | |
| 8.20 + | | Y | T | | | | | | H | | R | | R | | | | |
| V82T | | | | | | | | | | | | | | | | | |
| 8.20 + | | Y | T | | | | | | H | | T | | S | | | | |
| V82T + | | | | | | | | | | | | | | | | | |
| Y147T + | | | | | | | | | | | | | | | | | |
| Q154S | | | | | | | | | | | | | | | | | |
| ABE8e | | | | | S | R | N | N | | | D | Y | Q | | | I | N |
| ABE9.1e | 8.20 + | Y | T | | | | | | H | | R | Y | R | | | | |
| | V82T + | | | | | | | | | | | | | | | | |
| | F149Y | | | | | | | | | | | | | | | | |
| ABE9.2e | 8.20 + | Y | T | | | | | | H | | D | Y | R | | | I | N |
| | V82T + | | | | | | | | | | | | | | | | |
| | Y147D + | | | | | | | | | | | | | | | | |
| | F149Y + | | | | | | | | | | | | | | | | |
| | T166I + | | | | | | | | | | | | | | | | |
| | D167N | | | | | | | | | | | | | | | | |
| ABE- | | Y | G | | | | | | | | D | Y | S | | | | N |
| 301 | | | | | | | | | | | | | | | | | |
| TTR | 8.20 + | Y | S | | L | | | | H | | R | | R | S | | | |
| | A109L + | | | | | | | | | | | | | | | | |
| | V155S | | | | | | | | | | | | | | | | |
| TTR | F84Y + | Y | S | Y | | | | | H | | R | | R | | N | | |
| | F156N | | | | | | | | | | | | | | | | |

In particular embodiments, the fusion proteins or complexes comprise a single (e.g., provided as a monomer) TadA* (e.g., TadA*8 or TadA*9). Throughout the present disclosure, an adenosine deaminase base editor that comprises a single TadA* domain is indicates using the terminology ABEm or ABE #m, where “#” is an identifying number (e.g., ABE8.20m), where “m” indicates “monomer.” In some embodiments, the TadA* is linked to a Cas9 nickase. In some embodiments, the fusion proteins or complexes of the disclosure comprise as a heterodimer of a wild-type TadA (TadA(wt)) linked to a TadA*. Throughout the present disclosure, an adenosine deaminase base editor that comprises a single TadA* domain and a TadA(wt) domain is indicates using the terminology ABEd or ABE #d, where “#” is an identifying number (e.g., ABE8.20d), where “d” indicates “dimer.” In other embodiments, the fusion proteins or complexes of the disclosure comprise as a heterodimer of a TadA*7.10 linked to a TadA*. In some embodiments, the base editor is ABE8 comprising a TadA* variant monomer. In some embodiments, the base editor is ABE comprising a heterodimer of a TadA* and a TadA(wt). In some embodiments, the base editor is ABE comprising a heterodimer of a TadA* and TadA*7.10. In some embodiments, the base editor is ABE comprising a heterodimer of a TadA*. In some embodiments, the TadA* is selected from Tables 5A-5E.

In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation. Any of the mutations provided herein and any additional mutations (e.g., based on the ecTadA amino acid sequence) can be introduced into any other adenosine deaminases. Any of the mutations provided herein can be made individually or in any combination in a TadA reference sequence or another adenosine deaminase (e.g., ecTadA).

Details of A to G nucleobase editing proteins are described in International PCT Application No. PCT/US2017/045381 (WO2018/027078) and Gaudelli, N. M., et al., “Programmable base editing of A·T to G·C in genomic DNA without DNA cleavage” Nature, 551, 464-471 (2017), the entire contents of which are hereby incorporated by reference.

### C to T Editing

In some embodiments, a base editor disclosed herein comprises a fusion protein or complex comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C:G to a T: A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor can comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, the base editor can comprise a uracil stabilizing protein as described herein. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids.

In some embodiments, a cytidine deaminase of a base editor comprises all or a portion (e.g., a functional portion) of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase.

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Some aspects of the present disclosure are based on the recognition that modulating the deaminase domain catalytic activity of any of the fusion proteins or complexes described herein, for example by making point mutations in the deaminase domain, affect the processivity of the fusion proteins (e.g., base editors) or complexes. For example, mutations that reduce, but do not eliminate, the catalytic activity of a deaminase domain within a base editing fusion protein or complexes can make it less likely that the deaminase domain will catalyze the deamination of a residue adjacent to a target residue, thereby narrowing the deamination window. The ability to narrow the deamination window can prevent unwanted deamination of residues adjacent to specific target residues, which can reduce or prevent off-target effects.

In some embodiments, an APOBEC deaminase incorporated into a base editor can comprise one or more mutations selected from the group consisting of R33A, K34A, E63A, H102P, D104N, H121R, H122R, H122L, D124N; R126A, R126E, R118A, W90A, W90Y, and R132E of rAPOBEC1; D316R, D317R, R320A, R320E, R313A, W285A, W285Y, and R326E of hAPOBEC3G; and any alternative mutation at the corresponding position, or one or more corresponding mutations in another APOBEC deaminase. In some embodiments, an APOBEC deaminase incorporated into a base editor can comprise one or more combinations of mutations selected from K34A, H122L, and D124N (AALN); H102P and D104N (evoFERNY derived from FERNY); W90Y and R126E (YE1); W90Y and R132E (YE2); R126E and R132E (EE); W90Y, R126E, and R132E (YEE), or rAPOBEC1; and any alternative mutation at the corresponding positions, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion (e.g., a functional portion) of an APOBEC1 deaminase.

In some embodiments, the fusion proteins or complexes of the disclosure comprise one or more cytidine deaminase domains. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

In embodiments, a fusion protein of the disclosure comprises two or more nucleic acid editing domains.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference. Further non-limiting examples of C to T nucleobase editing proteins are described in PCT Applications No. PCT/US2020/062428 and PCT/US2019/033848, the entire contents of which are hereby incorporated by reference.

### Cytidine Adenosine Base Editors (CABEs)

In some embodiments, a base editor described herein comprises an adenosine deaminase variant that has increased cytidine deaminase activity. Such base editors may be referred to as “cytidine adenosine base editors (CABEs)” or “cytosine base editors derived from TadA* (CBE-Ts),” and their corresponding deaminase domains may be referred to as “TadA* acting on DNA cytosine (TADC)” domains or TadA-derived cytidine deaminases (TadA-CD). Base editors containing adenosine deaminase variants having both cytidine deaminase and adenosine deaminase activity (i.e., TadA-Dual deaminases) may be referred to as TadA-based dual editors (TadDE). In some instances, an adenosine deaminase variant has both adenine and cytosine deaminase activity (i.e., is a dual deaminase). In some embodiments, the adenosine deaminase variants deaminate adenine and cytosine in DNA. In some embodiments, the adenosine deaminase variants deaminate adenine and cytosine in single-stranded DNA. In some embodiments, the adenosine deaminase variants deaminate adenine and cytosine in RNA. In some embodiments, the adenosine deaminase variant predominantly deaminates cytosine in DNA and/or RNA (e.g., greater than 30%, 40%, 50%, 60%, 70%, 80%, 90%, 95%, or 99% of all deaminations catalyzed by the adenosine deaminase variant, or the number of cytosine deaminations catalyzed by the variant is about or at least about 2-fold, 3-fold, 4-fold, 5-fold, 6-fold, 7-fold, 8-fold, 9-fold, 10-fold, 25-fold, 50-fold, 75-fold, 100-fold, 500-fold, or 1,000-fold greater than the number adenine deaminations catalyzed by the variant). In some embodiments, the adenosine deaminase variant has approximately equal cytosine and adenosine deaminase activity (e.g., the two activities are within about 10% or 20% of each other). In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity. In some embodiments, the target polynucleotide is present in a cell in vitro or in vivo. In some embodiments, the cell is a bacteria, yeast, fungi, insect, plant, or mammalian cell. Examples of adenosine deaminase variants having increased cytidine deaminase activity include those described in International Patent Application Publications No. WO 2024/040083 and WO 2022/204574, the disclosures of which are hereby incorporated by reference in their entireties for all purposes.

In some embodiments, the CABE comprises a bacterial TadA deaminase variant (e.g., ecTadA). In some embodiments, the CABE comprises a truncated TadA deaminase variant. In some embodiments, the CABE comprises a fragment of a TadA deaminase variant. In some embodiments, the CABE comprises a TadA*8.20 variant.

In some embodiments, an adenosine deaminase variant of the disclosure is a TadA adenosine deaminase comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity (e.g., at least about 30%, 40%, 50% or more of the activity of a reference adenosine deaminase (e.g., TadA*8.20 or TadA*8.19)). In some instances, the adenosine deaminase variant comprises one or more alterations that increase cytosine deaminase activity (e.g., at least about 10-fold, 20-fold, 30-fold, 40-fold, 50-fold, 60-fold, 70-fold or more increase) relative to the activity of a reference adenosine deaminase and comprise undetectable adenosine deaminase activity or adenosine deaminase activity that is less than 30%, 20%, 10%, or 5% of that of a reference adenosine deaminase. In some embodiments, the reference adenosine deaminase is TadA*8.20 or TadA*8.19.

In some embodiments, the adenosine deaminase variant is an adenosine deaminase comprising two or more alterations at an amino acid position selected from the group consisting of 2, 4, 6, 8, 13, 17, 23, 27, 29, 30, 47, 48, 49, 67, 76, 77, 82, 84, 96, 100, 107, 112, 114, 115, 118, 119, 122, 127, 142, 143, 147, 149, 158, 159, 162 165, 166, and 167, of an amino acid sequence having at least about 70%, 75%, 80%, 85%, 90%, 95%, 98%, 99% or greater identity to SEQ ID NO: 1, or a corresponding alteration in another deaminase. I

In some embodiments, the adenosine deaminase variant is an adenosine deaminase comprising one or more alterations selected from the group consisting of S2H, V4K, V4S, V4T, V4Y, F6G, F6H, F6Y, H8Q, R13G, T17A, T17W, R23Q, E27C, E27G, E27H, E27K, E27Q, E27S, E27G, P29A, P29G, P29K, V30F, V30I, R47G, R47S, A48G, 149K, I49M, I49N, 149Q, 149T, G67W, I76H, I76R, I76W, Y76H, Y76R, Y76W, F84A, F84M, H96N, G100A, G100K, T111H, G112H, A114C, G115M, M118L, H122G, H122R, H122T, N127I, N127K, N127P, A142E, R147H, A158V, Q159S, A162C, A162N, A162Q, and S165P of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising an amino acid alteration or combination of amino acid alterations selected from those listed in any of Tables 6A-6F.

The residue identity of exemplary adenosine deaminase variants that are capable of deaminating adenine and/or cytidine in a target polynucleotide (e.g., DNA) is provided in Tables 6A-6F below. Further examples of adenosine deaminase variants include the following variants of 1.17 (see Table 6A): 1.17+E27H; 1.17+E27K; 1.17+E27S; 1.17+E27S+I49K; 1.17+E27G; 1.17+49N; 1.17+E27G+I49N; and 1.17+E27Q. In some embodiments, any of the amino acid alterations provided herein are substituted with a conservative amino acid. Additional mutations known in the art can be further added to any of the adenosine deaminase variants provided herein.

In some embodiments, the base editor systems comprising a CABE provided herein have at least about a 30%, 40%, 50%, 60%, 70% or more C to T editing activity in a target polynucleotide (e.g., DNA). In some embodiments, a base editor system comprising a CABE as provided herein has an increased C to T base editing activity (e.g., increased at least about 30-fold, 40-fold, 50-fold, 60-fold, 70-fold or more) relative to a reference base editor system comprising a reference adenosine deaminase (e.g., TadA*8.20 or TadA*8.19).

TABLE 6A
Adenosine Deaminase Variants. Mutations are indicated with reference to
TadA*8.20. “S” indicates “Surface,” and “NAS” indicates “Near Active Site.”

| | location in structure | | | | | | | | | | |
|---|---|---|---|---|---|---|---|---|---|---|---|
| | N/A | S h1 | S h1 | S h1 | NAS | NAS | NAS | NAS | | S | |
| | Amino Acid No. (*START Met is AA#1) | | | | | | | | | | |
| | 2 | 8 | 13 | 17 | 27 | 47 | 48 | 49 | 67 | 76 | 77 |
| TadA*8.20 | S | H | R | T | E | R | A | I | G | Y | D |
| TadA*8.19 | | | | | | | | | | I | |
| 1.1 | | | | | H | | | | | I | |
| 1.2 | | | | | H | | | K | | I | |
| 1.3 | | | | | S | | | K | | I | |
| 1.4 | | | | | S | | | K | | I | |
| 1.5 | | | | | K | | | | | | |
| 1.6 | | | | | K | | | | | | |
| 1.7 | | | | | H | | | | | I | |
| 1.8 | | | | | S | | | K | M | | |
| 1.9 | | | | | | | | T | W | | |
| 1.10 | | | | | C | | | | | I | |
| 1.11 | | | G | | O | | | | | | |
| 1.12 | | | | A | H | | | M | | I | |
| 1.13 | | | | | | | | Q | | I | |
| 1.14 | H | | | | | | | K | | I | |
| 1.15 | | | | | | S | | | | | |
| 1.16 | | Q | | | | | | Q | | I | |
| 1.17 | | | | A | | | G | | | | |
| 1.18 | | | | | G | | | | | | |
| 1.19 | | | | | G | | | N | | | |
| 1.20 | | | | | G | | | | | | G |

Adenosine Deaminase Variants. Mutations are indicated with
reference to TadA*8.20. “I” indicates “Internal,” “S” indicates “Surface,” and “NAS” indicates
“Near Active Site.”

| | location in structure | | | | | | | | | | | |
|---|---|---|---|---|---|---|---|---|---|---|---|---|
| | I | NAS | | NAS | S | | S | S | S | | S | |
| | Amino Acid No. (*START Met is AA#1) | | | | | | | | | | | |
| | 82 | 84 | 96 | 107 | 112 | 115 | 118 | 119 | 127 | 142 | 162 | 165 |
| TadA*8.20 | S | F | H | R | G | G | M | D | N | A | A | S |
| TadA*8.19 | | | | | | | | | | | | |
| 1.1 | | M | | | | | | | | | | |
| 1.2 | | | | | | | | | | | | |
| 1.3 | | | | | | | | | | | | |
| 1.4 | | | | | | | | | | | N | |
| 1.5 | | | | | | | | | | | | |
| 1.6 | | | | | | | | N | | | | |
| 1.7 | | | | | | | | | | | | |
| 1.8 | | | | | | | | | | | | |
| 1.9 | | | N | | | | | | | | | |
| 1.10 | | | | | | | | N | | | | |
| 1.11 | | | | | | | | | K | | | |
| 1.12 | | | | | | | L | | | | | |
| 1.13 | | | | | | M | | | | | | |
| 1.14 | | | | | H | | | | | | | |
| 1.15 | | | | C | | | | | | | | |
| 1.16 | | | | | | | | | | | | |
| 1.17 | T | | | | | | | | | E | | |
| 1.18 | | | | | | | | | | | | |
| 1.19 | | | | | | | | | | | | |
| 1.20 | | | | | | | | | | | | P |

TABLE 6B
Adenosine deaminase variants. Mutations are indicated
with reference to TadA*8.20.

| | Position No. | | | | | | | | | |
|---|---|---|---|---|---|---|---|---|---|---|
| | 27 | 29 | 30 | 49 | 82 | 84 | 107 | 112 | 115 | 142 |
| | TadA*8.20 | | | | | | | | | |
| | E | P | V | I | S | F | R | G | G | A |
| | Alterations Evaluated | | | | | | | | | |
| | G/S/H | G/A/K | I/L/F | K | T | L/A | C | H | M | E |
| S1.1 | S | | | K | T | | | | | |
| S1.2 | S | | | K | T | | C | | | |
| S1.3 | S | | | K | T | | | H | | |
| S1.4 | S | | | K | T | | | | M | |
| S1.5 | S | | | K | T | | | | | E |
| S1.6 | S | | | K | T | | C | H | | |
| S1.7 | S | | | K | T | | C | | M | |
| S1.8 | S | | | K | T | | C | | | |
| S1.9 | S | | | K | T | | | H | | E |
| S1.10 | S | | | K | T | | | | M | E |
| S1.11 | S | | | K | T | | C | H | M | E |
| S1.12 | S | | I | K | T | | | | | |
| S1.13 | S | | I | K | T | | C | | | |
| S1.14 | S | | I | K | T | | | H | | |
| S1.15 | S | | I | K | T | | | | M | |
| S1.16 | S | | I | K | T | | | | | E |
| S1.17 | S | | I | K | T | | C | H | | |
| S1.18 | S | | I | K | T | | C | | M | |
| S1.19 | S | | I | K | T | | C | | | E |
| S1.20 | S | | I | K | T | | | H | | E |
| S1.21 | S | | I | K | T | | | | M | E |
| S1.22 | S | | I | K | T | | C | H | M | E |
| S1.23 | S | | L | K | T | | | | | |
| S1.24 | S | | L | K | T | | C | | | |
| S1.25 | S | | L | K | T | | | H | | |
| S1.26 | S | | L | K | T | | | | M | |
| S1.27 | S | | L | K | T | | | | | E |
| S1.28 | S | | L | K | T | | C | H | | |
| S1.29 | S | | L | K | T | | | | M | |
| S1.30 | S | | L | K | T | | C | | | E |
| S1.31 | S | | L | K | T | | | H | | E |
| S1.32 | S | | L | K | T | | | | M | E |
| S1.33 | S | | L | K | T | | C | H | M | E |
| S1.34 | S | | F | K | T | A | | | | |
| S1.35 | S | | F | K | T | A | C | | | |
| S1.36 | S | | F | K | T | A | | H | | |
| S1.37 | S | | F | K | T | A | | | M | |
| S1.38 | S | | F | K | T | A | | | | E |
| S1.39 | S | | F | K | T | A | C | H | | |
| S1.40 | S | | F | K | T | A | C | | M | |
| S1.41 | S | | F | K | T | A | C | | | E |
| S1.42 | S | | F | K | T | A | | H | | E |
| S1.43 | S | | F | K | T | A | | | M | E |
| S1.44 | S | | F | K | T | A | C | H | M | E |
| S1.45 | S | | | K | T | L | | | | |
| S1.46 | S | | | K | T | L | C | | | |
| S1.47 | S | | | K | T | L | | H | | |
| S1.48 | S | | | K | T | L | | | M | |
| S1.49 | S | | | K | T | L | | | | E |
| S1.50 | S | | | K | T | L | C | H | | |
| S1.51 | S | | | K | T | L | C | | M | |
| S1.52 | S | | | K | T | L | C | | | E |
| S1.53 | S | | | K | T | L | | H | | |
| S1.54 | S | | | K | T | L | | | M | E |
| S1.55 | S | | | K | T | L | C | H | M | E |
| S1.56 | S | | I | K | T | L | | | | |
| S1.57 | S | | I | K | T | L | C | | | |
| S1.58 | S | | I | K | T | L | | H | | |
| S1.59 | S | | I | K | T | L | | | M | |
| S1.60 | S | | I | K | T | L | | | | E |
| S1.61 | S | | I | K | T | L | C | H | | |
| S1.62 | S | | I | K | T | L | C | | M | |
| S1.63 | S | | I | K | T | L | C | | | E |
| S1.64 | S | | I | K | T | L | | H | | |
| S1.65 | S | | I | K | T | L | | | M | E |
| S1.66 | S | | I | K | T | L | C | H | M | E |
| S1.67 | S | G | | K | T | | | | | |
| S1.68 | S | G | | K | T | | C | | | |
| S1.69 | S | G | | K | T | | | H | | |
| S1.70 | S | G | | K | T | | | | M | |
| S1.71 | S | G | | K | T | | | | | E |
| S1.72 | S | G | | K | T | | C | H | | |
| S1.73 | S | G | | K | T | | C | | M | |
| S1.74 | S | G | | K | T | | C | | | E |
| S1.75 | S | G | | K | T | | | H | | E |
| S1.76 | S | G | | K | T | | | | M | E |
| S1.77 | S | G | | K | T | | C | H | M | E |
| S1.78 | | G | | K | T | | | | | |
| S1.79 | | G | | K | T | | C | | | |
| S1.80 | | G | | K | T | | | H | | |
| S1.81 | | G | | K | T | | | | M | |
| S1.82 | | G | | K | T | | | | | E |
| S1.83 | | G | | K | T | | C | H | | |
| S1.84 | | G | | K | T | | C | | M | |
| S1.85 | | G | | K | T | | C | | | E |
| S1.86 | | G | | K | T | | | H | | |
| S1.87 | | G | | K | T | | | | M | E |
| S1.88 | | G | | K | T | | C | H | M | E |
| S1.89 | | K | | K | T | | | | | |
| S1.90 | | K | | K | T | | C | | | |
| S1.91 | | K | | K | T | | | H | | |
| S1.92 | | K | | K | T | | | | M | |
| S1.93 | | K | | K | T | | | | | E |
| S1.94 | | K | | K | T | | C | H | | |
| S1.95 | | K | | K | T | | C | | M | |
| S1.96 | | K | | K | T | | C | | | E |
| S1.97 | | K | | K | T | | | H | | E |
| S1.98 | | K | | K | T | | | | M | E |
| S1.99 | | K | | K | T | | C | H | M | E |
| S1.100 | | K | I | K | T | | | | | |
| S1.101 | | K | I | K | T | | C | | | |
| S1.102 | | K | I | K | T | | | H | | |
| S1.103 | | K | I | K | T | | | | M | |
| S1.104 | | K | I | K | T | | | | | E |
| S1.105 | | K | I | K | T | | C | H | | |
| S1.106 | | K | I | K | T | | C | | M | |
| S1.107 | | K | I | K | T | | C | | | E |
| S1.108 | | K | I | K | T | | | H | | |
| S1.109 | | K | I | K | T | | | | M | E |
| S1.110 | | K | I | K | T | | C | H | M | E |
| S1.111 | | K | | K | T | L | | | | |
| S1.112 | | K | | K | T | L | C | | | |
| S1.113 | | K | | K | T | L | | H | | |
| S1.114 | | K | | K | T | L | | | M | |
| S1.115 | | K | | K | T | L | | | | E |
| S1.116 | | K | | K | T | L | C | H | | |
| S1.117 | | K | | K | T | L | C | | M | |
| S1.118 | | K | | K | T | L | C | | | E |
| S1.119 | | K | | K | T | L | | H | | E |
| S1.120 | | K | | K | T | L | | | M | E |
| S1.121 | | K | | K | T | L | C | H | M | E |
| S1.122 | | K | I | K | T | L | | | | |
| S1.123 | | K | I | K | T | L | C | | | |
| S1.124 | | K | I | K | T | L | | H | | |
| S1.125 | | K | I | K | T | L | | | M | |
| S1.126 | | K | I | K | T | L | | | | E |
| S1.127 | | K | I | K | T | L | C | H | | |
| S1.128 | | K | I | K | T | L | C | | M | |
| S1.129 | | K | I | K | T | L | C | | | E |
| S1.130 | | K | I | K | T | L | | H | | E |
| S1.131 | | K | I | K | T | L | | | M | E |
| S1.132 | | K | I | K | T | L | C | H | M | E |
| S1.133 | G | | | K | T | | | | | |
| S1.134 | G | | | K | T | | C | | | |
| S1.135 | G | | | K | T | | | H | | |
| S1.136 | G | | | K | T | | | | M | |
| S1.137 | G | | | K | T | | | | | E |
| S1.138 | G | | | K | T | | C | H | | |
| S1.139 | G | | | K | T | | C | | M | |
| S1.140 | G | | | K | T | | C | | | E |
| S1.141 | G | | | K | T | | | H | | E |
| S1.142 | G | | | K | T | | | | M | E |
| S1.143 | G | | | K | T | | C | H | M | E |
| S1.144 | H | | | K | T | | | | | |
| S1.145 | H | | | K | T | | C | | | |
| S1.146 | H | | | K | T | | | H | | |
| S1.147 | H | | | K | T | | | | M | |
| S1.148 | H | | | K | T | | | | | E |
| S1.149 | H | | | K | T | | C | H | | |
| S1.150 | H | | | K | T | | C | | M | |
| S1.151 | H | | | K | T | | | | | E |
| S1.152 | H | | | K | T | | | H | | E |
| S1.153 | H | | | K | T | | | | M | E |
| S1.154 | H | | | K | T | | C | H | M | E |
| S1.155 | S | | | | T | | | | | |
| S1.156 | S | | | | T | | C | | | |
| S1.157 | S | | | | T | | | H | | |
| S1.158 | S | | | | T | | | | M | |
| S1.159 | S | | | | T | | | | | E |
| S1.160 | S | | | | T | | C | H | | |
| S1.161 | S | | | | T | | C | | M | |
| S1.162 | S | | | | T | | C | | | E |
| S1.163 | S | | | | T | | | H | | E |
| S1.164 | S | | | | T | | | | M | E |
| S1.165 | S | | | | T | | C | H | M | E |
| S1.166 | | A | | | T | | | | | |
| S1.167 | | A | | | T | | C | | | |
| S1.168 | | A | | | T | | | H | | |
| S1.169 | | A | | | T | | | | M | |
| S1.170 | | A | | | T | | | | | E |
| S1.171 | | A | | | T | | C | H | | |
| S1.172 | | A | | | T | | C | | M | |
| S1.173 | | A | | | T | | C | | | E |
| S1.174 | | A | | | T | | | H | | E |
| S1.175 | | A | | | T | | | | M | E |
| S1.176 | | A | | | T | | C | H | M | E |
| S1.177 | S | | I | | T | | | | | |
| S1.178 | S | | I | | T | | C | | | |
| S1.179 | S | | I | | T | | | H | | |
| S1.180 | S | | I | | T | | | | M | |
| S1.181 | S | | I | | T | | | | | E |
| S1.182 | S | | I | | T | | C | H | | |
| S1.183 | S | | I | | T | | C | | M | |
| S1.184 | S | | I | | T | | C | | | E |
| S1.185 | S | | I | | T | | | H | | E |
| S1.186 | S | | I | | T | | | | M | E |
| S1.187 | S | | I | | T | | C | H | M | E |
| S1.188 | | A | I | | T | L | | | | |
| S1.189 | | A | I | | T | L | C | | | |
| S1.190 | | A | I | | T | L | | H | | |
| S1.191 | | A | I | | T | L | | | M | |
| S1.192 | | A | I | | T | L | | | | E |
| S1.193 | | A | I | | T | L | C | H | | |
| S1.194 | | A | I | | T | L | C | | M | |
| S1.195 | | A | I | | T | L | C | | | E |
| S1.196 | | A | I | | T | L | | H | | E |
| S1.197 | | A | I | | T | L | | | M | E |
| S1.198 | | A | I | | T | L | C | H | M | E |
| S1.199 | S | A | L | K | T | L | C | H | M | E |

TABLE 6C
Adenosine deaminase variants. Mutations are indicated with reference to
variant 1.2 (Table 6A).

| | | Residue identity (START Met is amino | | | | | | | | |
|---|---|---|---|---|---|---|---|---|---|---|
| | | acid #1) | | | | | | | | |
| Variant Name | Alternative Variant Names | 4 | 6 | 17 | 23 | 76 | 77 | 100 | 111 | 114 |
| Reference | 1.2 (see Table 6A) | V | F | T | R | I | D | G | T | A |
| TadAC2.1 | pDKL-135; 2.1 | K | | | | | | | | C |
| TadAC2.2 | pDKL-136; 2.2 | K | | | | | G | | | |
| TadAC2.3 | pDKL-137; 2.3 | | Y | | | | | A | | |
| TadAC2.4 | pDKL-138; 2.4 | T | | | | R | | | | |
| TadAC2.5 | pDKL-139; 2.5 | | Y | | | W | | | | |
| TadAC2.6 | pDKL-140; 2.6 | | Y | | | | | | | |
| TadAC2.7 | pDKL-141; 2.7 | | Y | | | | | | | C |
| TadAC2.8 | pDKL-142; 2.8 | | Y | | | | | | | |
| TadAC2.9 | pDKL-143; 2.9 | K | | | | W | | | | |
| TadAC2.10 | pDKL-144; 2.10 | | G | | | R | | K | | |
| TadAC2.11 | pDKL-145; 2.11 | | H | | | | | | | |
| TadAC2.12 | pDKL-146; 2.12 | | | | | | | | | C |
| TadAC2.13 | pDKL-147; 2.13 | | Y | | | H | | | | |
| TadAC2.14 | pDKL-148; 2.14 | | | | | | | | | |
| TadAC2.15 | pDKL-149; 2.15 | | | | Q | R | | | | |
| TadAC2.16 | pDKL-150; 2.16 | | | | | H | | | | |
| TadAC2.17 | pDKL-151; 2.17 | | Y | | | | | | H | |
| TadAC2.18 | pDKL-152; 2.18 | | | | | W | | | | |
| TadAC2.19 | pDKL-153; 2.19 | | | | | | | | H | |
| TadAC2.20 | pDKL-154; 2.20 | | | | | | | | | |
| TadAC2.21 | pDKL-155; 2.21 | | Y | | | R | | | | |
| TadAC2.22 | pDKL-156; 2.22 | | | W | | H | | | | |
| TadAC2.23 | pDKL-157; 2.23 | S | | | | Y | | | | |
| TadAC2.24 | pDKL-158; 2.24 | | | | | | | | | |
| | | Residue identity (START Met is amino acid #1) | | | | | | | | |
| Variant Name | Alternative Variant Names | 119 | 122 | 127 | 143 | 147 | 158 | 159 | 162 | 166 |
| Reference | 1.2 (see Table 6A) | D | H | N | A | R | A | Q | A | T |
| TadAC2.1 | pDKL-135; 2.1 | | | | | | | | | |
| TadAC2.2 | pDKL-136; 2.2 | | | | | | | | | |
| TadAC2.3 | pDKL-137; 2.3 | | R | | | | | | | |
| TadAC2.4 | pDKL-138; 2.4 | | G | | | | | | | |
| TadAC2.5 | pDKL-139; 2.5 | | | | | | | | | |
| TadAC2.6 | pDKL-140; 2.6 | N | | | | | | | | |
| TadAC2.7 | pDKL-141; 2.7 | | | | | | | | | |
| TadAC2.8 | pDKL-142; 2.8 | | | | | | | | | |
| TadAC2.9 | pDKL-143; 2.9 | | T | | | | | | | |
| TadAC2.10 | pDKL-144; 2.10 | | | | | | | | | |
| TadAC2.11 | pDKL-145; 2.11 | | N | | | | | | | |
| TadAC2.12 | pDKL-146; 2.12 | | | | | | | | | |
| TadAC2.13 | pDKL-147; 2.13 | | R | | | | | | | I |
| TadAC2.14 | pDKL-148; 2.14 | | | P | | | | | | |
| TadAC2.15 | pDKL-149; 2.15 | | | | | | | | | |
| TadAC2.16 | pDKL-150; 2.16 | | R | | | | V | | | |
| TadAC2.17 | pDKL-151; 2.17 | | | | | | | | | |
| TadAC2.18 | pDKL-152; 2.18 | | | | | | | | | |
| TadAC2.19 | pDKL-153; 2.19 | | G | | | | | | C | |
| TadAC2.20 | pDKL-154; 2.20 | | | | E | | | | | |
| TadAC2.21 | pDKL-155; 2.21 | | | | | | | | | |
| TadAC2.22 | pDKL-156; 2.22 | | G | | | | V | | | |
| TadAC2.23 | pDKL-157; 2.23 | | | | E | | | S | | |
| TadAC2.24 | pDKL-158; 2.24 | | | I | | | | | Q | |

TABLE 6D
Adenosine deaminase variants. Mutations are indicated with reference to TadA*8.20.

| | AA Positions | | | | | | | | | | | | | | |
|---|---|---|---|---|---|---|---|---|---|---|---|---|---|---|---|
| | 6 | 27 | 49 | 76 | 77 | 82 | 107 | 112 | 114 | 115 | 119 | 122 | 127 | 142 | 143 |
| | TadA*8.20 | | | | | | | | | | | | | | |
| | F | E | I | Y | D | S | R | G | A | G | D | H | N | A | A |
| S1.154 | F | H | K | Y | D | T | C | H | | M | | | | E | |
| Alterations from Table 6C | Y | | | M | G | | | | C | | N | G | | | E |
| S2.1 | Y | H | K | W | | T | C | H | | M | | | | E | |
| S2.2 | Y | H | K | | G | T | C | H | | M | | | | E | |
| S2.3 | Y | H | K | | | T | C | H | C | M | | | | E | |
| S2.4 | Y | H | K | | | T | C | H | | M | N | | | E | |
| S2.5 | Y | H | K | | | T | C | H | | M | | G | | E | |
| S2.6 | Y | H | K | | | T | C | H | | M | | | P | E | |
| S2.7 | Y | H | K | | | T | C | H | | M | | | | E | E |
| S2.8 | Y | H | K | | | T | C | H | | M | | | | A | E |
| S2.9 | Y | H | K | W | G | T | C | H | | M | | | | E | |
| S2.10 | Y | H | K | W | | T | C | H | C | M | | | | E | |
| S2.11 | Y | H | K | W | | T | C | H | | M | N | | | | |
| S2.12 | Y | H | K | W | | T | C | H | | M | | G | | E | |
| S2.13 | Y | H | K | W | | T | C | H | | M | | | P | E | |
| S2.14 | Y | H | K | W | | T | C | H | | M | | | | E | E |
| S2.15 | Y | H | K | W | | T | C | H | | M | | | | A | E |
| S2.16 | Y | H | K | | G | T | C | H | C | M | | | | E | |
| S2.17 | Y | H | K | | G | T | C | H | | M | N | | | E | |
| S2.18 | Y | H | K | | G | T | C | H | | M | | G | | E | |
| S2.19 | Y | H | K | | G | T | C | H | | M | | | P | E | |
| S2.20 | Y | H | K | | G | T | C | H | | M | | | | E | E |
| S2.21 | Y | H | K | | G | T | C | H | | M | | | | A | E |
| S2.22 | Y | H | K | | | T | C | H | C | M | N | | | E | |
| S2.23 | Y | H | K | | | T | C | H | C | M | | G | | E | |
| S2.24 | Y | H | K | | | T | C | H | C | M | | | P | E | |
| S2.25 | Y | H | K | | | T | C | H | | M | N | G | | E | |
| S2.26 | Y | H | K | | | T | C | H | | M | N | | P | E | |
| S2.27 | Y | H | K | | | T | C | H | | M | | G | P | E | |
| S2.28 | Y | H | K | W | G | T | C | H | C | M | | | | E | |
| S2.29 | Y | H | K | W | G | T | C | H | | M | N | | | E | |
| S2.30 | Y | H | K | M | G | T | C | H | | M | | G | | E | |
| S2.31 | Y | H | K | M | G | T | C | H | | M | | | P | E | |
| S2.32 | Y | H | K | W | G | T | C | H | | M | | | | E | E |
| S2.33 | Y | H | K | W | G | T | C | H | | M | | | | A | E |
| S2.34 | Y | H | K | W | | T | C | H | C | M | N | | | E | |
| S2.35 | Y | H | K | W | | T | C | H | C | M | | G | | E | |
| S2.36 | Y | H | K | W | | T | C | H | C | M | | | P | E | |
| S2.37 | Y | H | K | M | | T | C | H | C | M | | | | E | E |
| S2.38 | Y | H | K | W | | T | C | H | C | M | | | | A | E |
| S2.39 | Y | H | K | W | | T | C | H | | M | N | G | | E | |
| S2.40 | Y | H | K | W | | T | C | H | | M | N | | P | E | |
| S2.41 | Y | H | K | W | | T | C | H | | M | | G | P | E | |
| S2.42 | Y | H | K | M | | T | C | H | C | M | N | G | | E | |
| S2.43 | Y | H | K | W | | T | C | H | C | M | N | | P | E | |
| S2.44 | T | H | K | W | | T | C | H | C | M | | G | P | E | |
| S2.45 | Y | H | K | W | G | T | C | H | C | M | N | | | E | |
| S2.46 | Y | H | K | W | G | T | C | H | C | M | | G | | E | |
| S2.47 | Y | H | K | W | G | T | C | H | C | M | | | P | E | |
| S2.48 | Y | H | K | W | G | T | C | H | C | M | | | | E | E |
| S2.49 | Y | H | K | W | G | T | C | H | C | M | | | | A | E |
| S2.50 | Y | H | K | W | G | T | C | H | C | M | N | G | | E | |
| S2.51 | Y | H | K | M | G | T | C | H | C | M | N | | P | E | |
| S2.52 | Y | H | K | W | G | T | C | H | C | M | | G | P | E | |
| S2.53 | Y | H | K | W | | T | C | H | C | M | N | G | P | E | E |
| S2.54 | Y | H | K | M | | T | C | H | C | M | N | G | P | A | E |
| S2.55 | Y | H | K | W | G | T | C | H | C | M | N | G | P | E | E |
| S2.56 | Y | H | K | W | G | T | C | H | C | M | N | G | P | A | E |

TABLE 6E
Hybrid constructs. Mutations are indicated with reference to TadA*7.10.

| | TadA amino acid subsitutions | | | | | | | | | | | |
|---|---|---|---|---|---|---|---|---|---|---|---|---|
| | 76 | 82 | 109 | 111 | 119 | 122 | 123 | 147 | 149 | 154 | 166 | 167 |
| TadA*7.10 | I | V | A | T | D | H | Y | Y | F | Q | T | D |
| TadA*8e | | | S | R | N | N | | D | Y | | I | N |
| TadA*8.20 | Y | S | | | | | H | R | | R | | |
| TadA*8.17 | | S | | | | | | | | R | | |
| pNMG-B878 | Y | S | | | | | H | D | | R | | |
| pNMG-B879 | Y | S | | | | | H | R | Y | R | | |
| pNMG-B880 | Y | S | | | | | H | R | | R | I | |
| pNMG-B881 | Y | S | | | | | H | R | | R | | N |
| pNMG-B882 | Y | S | | | | | H | D | Y | R | I | N |
| pNMG-B883 | Y | S | | R | N | | H | R | | R | | |
| pNMG-B884 | Y | S | S | R | N | N | H | R | | R | | |
| pNMG-B885 | Y | S | S | | | | H | R | | R | | |
| pNMG-B886 | Y | S | | R | | | H | R | | R | | |
| pNMG-B887 | Y | S | | | N | | H | R | | R | | |
| pNMG-B888 | Y | S | | | | N | H | R | | R | | |
| pNMG-B889 | Y | S | S | R | | | H | R | | R | | |
| pNMG-B890 | Y | S | | | N | N | H | R | | R | | |
| pNMG-B891 | Y | S | S | R | N | N | H | D | Y | R | I | N |

TABLE 6F
Base editor variants. Mutations are indicated with reference to TadA*8.19/8.20.

| AA positions: | 17 | 27 | 48 | 49 | 76 | 82 | 84 | 118 | 142 | 147 | 149 | 166 | 167 |
|---|---|---|---|---|---|---|---|---|---|---|---|---|---|
| ABE8.19m/8.20m | T | E | A | I | Y/I | S | F | M | A | Y | F | T | D |
| 1.1 + 8e(B879) | | H | | | I | | M | | | | Y | | |
| 1.2 + 8e(B879) | | H | | K | I | | | | | | Y | | |
| 1.12 + 8e(B879) | A | H | | M | I | | | L | | | Y | | |
| 1.17 + 8e(B879) | A | | G | | | T | | | E | | Y | | |
| 1.18 + 8e(B879) | | G | | | | | | | | | Y | | |
| 1.19 + 8e(B879) | | G | | N | | | | | | | Y | | |
| 1.1 + 8e(B882) | | H | | | I | | M | | | D | Y | I | N |
| 1.2 + 8e(B882) | | H | | K | I | | | | | D | Y | I | N |
| 1.12 + 8e(B882) | A | H | | M | I | | | L | | D | Y | I | N |
| 1.17 + 8e(B882) | A | | G | | | T | | | E | D | Y | I | N |
| 1.18 + 8e(B882) | | G | | | | | | | | D | Y | I | N |
| 1.19 + 8e(B882) | | G | | N | | | | | | D | Y | I | N |

A TadA-derived cytidine deaminase (e.g., TadA-CD), according to certain embodiments, comprises an amino acid sequence that is at least 80% identical, at least 85% identical, at least 90% identical, at least 95% identical, at least 98% identical, at least 99% identical, and at least 99.5% identical to the amino acid sequence of SEQ ID NO: 3594, wherein residue 27 of SEQ ID NO: 3594 is any amino acid expect for E (glutamic acid). TadA-CDs with other sequence homologies are also possible. For example, in certain embodiments, the TadA-derived cytidine deaminase (e.g., TadA-CD) comprises an amino acid sequence that is at least 80% identical, at least 85% identical, at least 90% identical, at least 95% identical, at least 98% identical, at least 99% identical, and at least 99.5% identical to the amino acid sequence of SEQ ID NO: 3594, wherein residue 28 of SEQ ID NO: 3594 is any amino acid expect for V (valine). In another exemplary embodiment, the TadA-derived cytidine deaminase is at least 80% identical, at least 85% identical, at least 90% identical, at least 95% identical, at least 98% identical, at least 99% identical, and at least 99.5% identical to the amino acid sequence of SEQ ID NO: 3594, wherein residue 96 of SEQ ID NO: 3594 is any amino acid expect for H (histidine). In another exemplary embodiment, the TadA-derived cytidine deaminase is at least 80% identical, at least 85% identical, at least 90% identical, at least 95% identical, at least 98% identical, at least 99% identical, and at least 99.5% identical to the amino acid sequence of SEQ ID NO: 3594, wherein residue 26 of SEQ ID NO: 3594 is any amino acid expect for R (arginine). In various embodiments, the TadA-derived cytidine deaminase comprises an alteration at one or more of positions 26, 27, 28, 48, 73, or 96 compared to SEQ ID NO: 3594.

As will be appreciated by those of skill in the art, TadA-derived cytidine deaminases (e.g., TadA-CD) may comprise a plurality of mutations relative to the parent adenosine deaminase (e.g., TadA-8e). In some embodiments, the deaminase of the instant application (e.g., TadA-CD) comprises mutations at residues E27, V28, and H96. In some embodiments, the disclosed deaminase further comprises at least one mutation at a residue selected from R26, M61, Y73, 176, M151, Q154, and A158, in the amino acid sequence of SEQ ID NO: 3594, or corresponding mutations in a homologous adenosine deaminase.

In some embodiments, the deaminase comprises at least one mutation selected from E27A, E27K, V28G, V28A, and H96N, and further comprises at least one mutation at a residue selected from R26G, M61I, Y73H, Y73S, Y73C, I76F, MISII, Q154R, Q154H, and A158S, in the amino acid sequence of SEQ ID NO: 3594, or a corresponding mutation in a homologous adenosine deaminase. Other mutations are also possible. For example, in certain embodiments, the TadA-CD enzyme comprises mutations selected from E27A, V28G, and H96N, and further comprises at least one mutation selected from R26G, M61I, Y73H, Y73S, Y73C, 176F, M151I, Q154R, Q154H, and A158S, in the amino acid sequence of SEQ ID NO: 3594, or corresponding mutations in a homologous adenosine deaminase.

Other exemplary embodiments may include (1) deaminases comprising mutations E27K, V28G, and H96N, and further comprising at least one mutation selected from R26G, M611, Y73H, Y73S, Y73C, 176F, M151I, Q154R, Q154H, and A158S, in the amino acid sequence of SEQ ID NO: 3594 or corresponding mutations in a homologous adenosine deaminase; (2) deaminases comprising mutations E27A, V28A, and H96N, and further comprising at least one mutation selected from R26G, M61I, Y73H, Y73S, Y73C, 176F, M151I, Q154R, Q154H, and A158S, in the amino acid sequence of SEQ ID NO: 3594, or corresponding mutations in a homologous adenosine deaminase; (3) deaminases comprising mutations E27K, V28A, and H96N, and further comprising at least one mutation selected from R26G, M611, Y73H, Y73S, Y73C, 176F, M151I, Q154R, Q154H, and A158S, in the amino acid sequence of SEQ ID NO: 3594, or corresponding mutations in a homologous adenosine deaminase.

In some embodiments, the TadA-derived cytidine deaminases (TadA-CD) comprise at least two mutations at residues selected from R26, M61, Y73, 176, M151, Q154, and A158 (relative to a reference adenosine deaminase). In other embodiments, the TadA-CD comprises at least two mutations at residues selected from R26G, M611, Y73H, 176F, M151I, Q154H, Q154R, and A158S.

In some embodiments, the addition of a V106W mutation improves the selectivity by suppressing A deamination to a greater extent than C deamination.

In some embodiments, a TadA-based dual editor comprises an adenosine deaminase variant comprising one, two, three, four, or five mutations selected from R26G, V28A, A48R, Y73S, and H96N (e.g., SEQ ID NO: 3600).

As such, in some embodiments, provided herein are deaminases that comprise mutations at residues R26, V28, A48, and Y73 in the amino acid sequence of SEQ ID NO: 3594, or corresponding mutations in a homologous adenosine deaminase. Further provided herein are deaminases that comprise mutations at residues R26, E27, V28, A48, and Y73 (e.g., further comprise a mutation at E27) in the amino acid sequence of SEQ ID NO: 3594. In particular embodiments, these deaminases comprise the mutations R26G, V28A, A48R, Y73S, and H96N. In some embodiments, these deaminases comprise the mutations R26G, V28G, A48R, and Y73C.

TadA-CD variants may comprise at least one mutation selected from R26G, E27A, V28G, I76F, H96N, and M151I (e.g, TadA-CDa, SEQ ID NO: 3595); R26G, E27A, V28G, I76F, H96N, and A158S (e.g, TadA-CDb, SEQ ID NO: 3596); R26G, E27A, V28G, 176F, H96N, Q154R, and A158S (e.g, TadA-CDc, SEQ ID NO: 3597); E27A, V28G, Y73H, H96N, Q154H, and A158S (e.g., TadA-CDd, SEQ ID NO: 3598); R26G, V28A, A48R, Y73S, and H96N (e.g., TadA-CDe, SEQ ID NO: 3599); V28A, A48R, and Y73S (e.g, TadA-CDf, SEQ ID NO: 3600), and R26G, V28G, A48R, and Y73C (e.g, TadA-CDg, SEQ ID NO: 3601).

In some preferred embodiments, the deaminase comprises the mutations R26G, E27A, V28G, 176F, H96N, and A158S (e.g., TadA-CDa, SEQ ID NO: 3595), R26G, E27A, V28G, I76F, H96N, Q154R, and A158S (e.g., TadA-CDb, SEQ ID NO: 3596), R26G, E27A, V28G, 176F, H96N, and M151I (e.g., TadA-CDc, SEQ ID NO: 3597), E27K, V28A, M61I, and H96N (e.g., TadA-CDd, SEQ ID NO: 3598), E27A, V28G, Y73H, H96N, Q154H, and A158S (e.g., TadA-CDe, SEQ ID NO: 3599), R26G, V28A, A48R, Y73S, and H96N (e.g., TadA-CDf, SEQ ID NO: 3600), and R26G, V28G, A48R, and Y73C (e.g., TadA-CDg, SEQ ID NO: 3601).

In some embodiments, the TadA-CD variants described above and herein may also comprises a V106W mutation.

In some embodiments, the TadA-CD variants comprise at least 80%, at least 85%, at least 90%, at least 95%, at least 98%, at least 99%, or at least 99.5% to any of the amino acid sequences of SEQ ID NOs: 3594-3601.

In some embodiments, the evolved TadA-Dual deaminase comprises the mutations R26G, V28A, N46I, A48R, Y73P, and H96N (TadA-CD-1, SEQ ID NO: 3602) relative to the amino acid sequence of SEQ ID NO: 3594. In some embodiments, the evolved TadA-Dual deaminase comprises the mutations R26G, V28A, N46T, A48R, Y73P, and H96N (TadA-CD-2, SEQ ID NO: 3603) relative to the amino acid sequence of SEQ ID NO: 3594. In some embodiments, the evolved TadA-Dual deaminase comprises the mutations R26G, V28A, N46T, A48R, Y73S, and H96N (TadA-CD-3, SEQ ID NO: 3604) relative to the amino acid sequence of SEQ ID NO: 3594. In some embodiments, the evolved TadA-Dual deaminase comprises the mutations R26G, V28A, N46V, A48R, Y73S, and H96N (TadA-CD-4, SEQ ID NO: 3605) relative to the amino acid sequence of SEQ ID NO: 3594. In some embodiments, the evolved TadA-Dual deaminase comprises the mutations R26G, V28A, N46V, A48R, Y73P, and H96N (TadA-CD-5, SEQ ID NO: 3606) relative to the amino acid sequence of SEQ ID NO: 3594. In some embodiments, the evolved TadA-Dual deaminase comprises the mutations R26G, V28A, N46L, A48R, Y73P, and H96N (TadA-CD-6, SEQ ID NO: 3607) relative to the amino acid sequence of SEQ ID NO: 3594. In some embodiments, the evolved TadA-Dual deaminase comprises the mutations V28A, N46L, A48P, and Y73P (TadA-CD-7, SEQ ID NO: 3608) relative to the amino acid sequence of SEQ ID NO: 3594. In some embodiments, the evolved TadA-Dual deaminase comprises the mutations V28A, N46C, A48P, and Y73P (TadA-CD-8, SEQ ID NO: 3609) relative to the amino acid sequence of SEQ ID NO: 3594. In some embodiments, the evolved TadA-Dual deaminase comprises the mutations R26G, V28A, N46V, A48R, Y73P, and H96N (TadA-CD-9, SEQ ID NO: 3610) relative to the amino acid sequence of SEQ ID NO: 3594. In some embodiments, the evolved TadA-Dual deaminase comprises the mutations R26G, V28A, N46V, A48R, Q71H, Y73P, and H96N (TadA-CD-10, SEQ ID NO: 3611) relative to the amino acid sequence of SEQ ID NO: 3594. In some embodiments, the evolved TadA-Dual deaminase comprises the mutations R26G, V28A, N46L, A48R, Y73P, and H96N (TadA-CD-11, SEQ ID NO: 3612) relative to the amino acid sequence of SEQ ID NO: 3594. In some embodiments, the evolved TadA-Dual deaminase comprises the mutations R26G, V28A, N46C, A48R, Y73P, and H96N (TadA-CD-12, SEQ ID NO: 3613) relative to the amino acid sequence of SEQ ID NO: 3594. In some embodiments, the evolved TadA-Dual deaminase comprises the mutations R26G, V28A, N46C, A48R, Y73P, H96N, and A162V (TadA-CD-13, SEQ ID NO: 3614) relative to the amino acid sequence of SEQ ID NO: 3594.

In some embodiments, the evolved TadA-Dual deaminase comprises the mutations R26G, V28A, N461, A48R, Y73S, and H96N (TadA-CD-14, SEQ ID NO: 3615) relative to the amino acid sequence of SEQ ID NO: 3594. In some embodiments, the evolved TadA-Dual deaminase comprises the mutations R26G, V28A, A48R, Q71S, Y73S, and H96N (TadA-CD-15, SEQ ID NO: 3616) relative to the amino acid sequence of SEQ ID NO: 3594. In some embodiments, the evolved TadA-Dual deaminase comprises the mutations R26G, V28A, N46L, A48R, and Y73P (TadA-CD-16, SEQ ID NO: 3617) relative to the amino acid sequence of SEQ ID NO: 3594. In some embodiments, the evolved TadA-Dual deaminase comprises the mutations R26G, V28A, N46L, A48R, Y73P, and H96N (TadA-CD-17, SEQ ID NO: 3618) relative to the amino acid sequence of SEQ ID NO: 3594. In some embodiments, the evolved TadA-Dual deaminase comprises the mutations R26G, V28A, Y73P, and H96N (TadA-CD-18, SEQ ID NO: 3619) relative to the amino acid sequence of SEQ ID NO: 3594. In some embodiments, the evolved TadA-Dual deaminase comprises the mutations R26G, V28A, N46V, A48R, Y73S, and H96N (TadA-CD-19, SEQ ID NO: 3620) relative to the amino acid sequence of SEQ ID NO: 3594. In some embodiments, the evolved TadA-Dual deaminase comprises the mutations R26G, V28A, N46V, A48R, Y73P, and H96N (TadA-CD-20, SEQ ID NO: 3621) relative to the amino acid sequence of SEQ ID NO: 3594. In some embodiments, the evolved TadA-Dual deaminase comprises the mutations R26G and N46L (TadA-CD-21, SEQ ID NO: 3622) relative to the amino acid sequence of SEQ ID NO: 3594. In some embodiments, the evolved TadA-Dual deaminase comprises the mutations R26G, V28A, N46I, A48R, Y73P, and H96N (TadA-CD-22, SEQ ID NO: 3623) relative to the amino acid sequence of SEQ ID NO: 3594. In some embodiments, the evolved TadA-Dual deaminase comprises the mutations R26G, V28A, N46V, A48R, Y73P, and H96N (TadA-CD-23, SEQ ID NO: 3624) relative to the amino acid sequence of SEQ ID NO: 3594. In some embodiments, the evolved TadA-Dual deaminase comprises the mutations R26G, V28A, A48P, Y73H, T79P, and H96N (TadA-CD-24, SEQ ID NO: 3625) relative to the amino acid sequence of SEQ ID NO: 3594. In some embodiments, the evolved TadA-Dual deaminase comprises the mutations R26G, N46I, and H96N (TadA-CD-25, SEQ ID NO: 3626) relative to the amino acid sequence of SEQ ID NO: 3594.

In some embodiments, the evolved TadA-Dual deaminase comprises the mutations R26G, V28A, N46V, A48R, Y73P, and H96N (TadA-CD-26, SEQ ID NO: 3627) relative to the amino acid sequence of SEQ ID NO: 3594. In some embodiments, the evolved TadA-Dual deaminase comprises the mutations R26G, V28A, N46L, A48R, Y73S, and H96N (TadA-CD-27, SEQ ID NO: 3628) relative to the amino acid sequence of SEQ ID NO: 3594. In some embodiments, the evolved TadA-Dual deaminase comprises the mutations R26G, V28A, N46C, A48R, H96N, and A162V (TadA-CD-28, SEQ ID NO: 3629) relative to the amino acid sequence of SEQ ID NO: 3594. In some embodiments, the evolved TadA-Dual deaminase comprises the mutations R26G, V28A, N46V, A48R, Q71H, Y73P, and H96N (TadA-CD-29, SEQ ID NO: 3630) relative to the amino acid sequence of SEQ ID NO: 3594. In some embodiments, the evolved TadA-Dual deaminase comprises the mutations R26G, V28A, N46C, A48R, Y73P, and H96N (TadA-CD-30, SEQ ID NO: 3631) relative to the amino acid sequence of SEQ ID NO: 3594. In some embodiments, the evolved TadA-Dual deaminase comprises the mutations R26G, V28A, N46C, A48R, Y73P, H96N, and A162V (TadA-CD-31, SEQ ID NO: 3632) relative to the amino acid sequence of SEQ ID NO: 3594.

In some embodiments, the evolved TadA-Dual deaminase comprises the mutations R26G, V28A, N46V, A48R, Y73P, and H96N (TadA-CD-32, SEQ ID NO: 3633) relative to the amino acid sequence of SEQ ID NO: 3594. In some embodiments, the evolved TadA-Dual deaminase comprises the mutations R26G, V28A, N46V, A48R, Y73S, and H96N (TadA-CD-33, SEQ ID NO: 3634) relative to the amino acid sequence of SEQ ID NO: 3594. In some embodiments, the evolved TadA-Dual deaminase comprises the mutations R26G, V28A, N46V, A48P, Y73S, and H96N (TadA-CD-34, SEQ ID NO: 3635) relative to the amino acid sequence of SEQ ID NO: 3594. In some embodiments, the evolved TadA-Dual deaminase comprises the mutations R26G, V28A, N46C, A48R, Y73P, and H96N (TadA-CD-35, SEQ ID NO: 3636) relative to the amino acid sequence of SEQ ID NO: 3594. In some embodiments, the evolved TadA-Dual deaminase comprises the mutations R26G, V28A, L34M, N46L, A48R, Y73P, and H96N (TadA-CD-36, SEQ ID NO: 3637) relative to the amino acid sequence of SEQ ID NO: 3594. In some embodiments, the evolved TadA-Dual deaminase comprises the mutations R26G, V28A, N46L, A48R, Y73P, and H96N (TadA-CD-37, SEQ ID NO: 3638) relative to the amino acid sequence of SEQ ID NO: 3594. In some embodiments, the evolved TadA-Dual deaminase comprises the mutations R26G, V28A, N46L, A48P, R64K, Y73P, and H96N (TadA-CD-38, SEQ ID NO: 3639) relative to the amino acid sequence of SEQ ID NO: 3594.

In some embodiments, the evolved TadA-Dual deaminase comprises the mutations N46I, S73P, and H154Q (TadA-CD-1, SEQ ID NO: 3602) relative to the amino acid sequence of SEQ ID NO: 3600. In some embodiments, the evolved TadA-Dual deaminase comprises the mutations N46T (TadA-CD-2, SEQ ID NO: 3603) relative to the amino acid sequence of SEQ ID NO: 3600. In some embodiments, the evolved TadA-Dual deaminase comprises the mutations N46T and H154Q (TadA-CD-3, SEQ ID NO: 3604) relative to the amino acid sequence of SEQ ID NO: 3600. In some embodiments, the evolved TadA-Dual deaminase comprises the mutations N46V and H154Q (TadA-CD-4, SEQ ID NO: 3605) relative to the amino acid sequence of SEQ ID NO: 3600. In some embodiments, the evolved TadA-Dual deaminase comprises the mutations N46V, S73P, G105S, and H154Q (TadA-CD-5, SEQ ID NO: 3606) relative to the amino acid sequence of SEQ ID NO: 3600. In some embodiments, the evolved TadA-Dual deaminase comprises the mutations N46L, S73P, and H154Q (TadA-CD-6, SEQ ID NO: 3607) relative to the amino acid sequence of SEQ ID NO: 3600. In some embodiments, the evolved TadA-Dual deaminase comprises the mutations G26R N46L, R48P, S73P, N96H, and H154Q (TadA-CD-7, SEQ ID NO: 3608) relative to the amino acid sequence of SEQ ID NO: 3600. In some embodiments, the evolved TadA-Dual deaminase comprises the mutations N46C, N96H, and H154Q (TadA-CD-8, SEQ ID NO: 3609) relative to the amino acid sequence of SEQ ID NO: 3600. In some embodiments, the evolved TadA-Dual deaminase comprises the mutations N46V, S73P, and H154Q (TadA-CD-9, SEQ ID NO: 3610) relative to the amino acid sequence of SEQ ID NO: 3600. In some embodiments, the evolved TadA-Dual deaminase comprises the mutations N46V, Q71H, S73P, and H154Q (TadA-CD-10, SEQ ID NO: 3611) relative to the amino acid sequence of SEQ ID NO: 3600. In some embodiments, the evolved TadA-Dual deaminase comprises the mutations N46L and H154Q (TadA-CD-11, SEQ ID NO: 3612) relative to the amino acid sequence of SEQ ID NO: 3600. In some embodiments, the evolved TadA-Dual deaminase comprises the mutations N46C, S73P, and H154Q (TadA-CD-12, SEQ ID NO: 3613) relative to the amino acid sequence of SEQ ID NO: 3600. In some embodiments, the evolved TadA-Dual deaminase comprises the mutations N46C, S73P, H154Q, and A162V (TadA-CD-13, SEQ ID NO: 3614) relative to the amino acid sequence of SEQ ID NO: 3600.

In some embodiments, the evolved TadA-Dual deaminase comprises the mutations N461 and H154Q (TadA-CD-14, SEQ ID NO: 3615) relative to the amino acid sequence of SEQ ID NO: 3600. In some embodiments, the evolved TadA-Dual deaminase comprises the mutations Q71S and H154Q (TadA-CD-15, SEQ ID NO: 3616) relative to the amino acid sequence of SEQ ID NO: 3594. In some embodiments, the evolved TadA-Dual deaminase comprises the mutations N46L, S73P, N79T, and N96H (TadA-CD-16, SEQ ID NO: 3617) relative to the amino acid sequence of SEQ ID NO: 3600. In some embodiments, the evolved TadA-Dual deaminase comprises the mutations N46L, S73P, N79T (TadA-CD-17, SEQ ID NO: 3618) relative to the amino acid sequence of SEQ ID NO: 3600. In some embodiments, the evolved TadA-Dual deaminase comprises the mutations R48A, S73P, and N79T (TadA-CD-18, SEQ ID NO: 3619) relative to the amino acid sequence of SEQ ID NO: 3600. In some embodiments, the evolved TadA-Dual deaminase comprises the mutations N46V and N79T (TadA-CD-19, SEQ ID NO: 3620) relative to the amino acid sequence of SEQ ID NO: 3600. In some embodiments, the evolved TadA-Dual deaminase comprises the mutations N46V, S73P, and N79T (TadA-CD-20, SEQ ID NO: 3621) relative to the amino acid sequence of SEQ ID NO: 3600. In some embodiments, the evolved TadA-Dual deaminase comprises the mutations A28V, N46L, R48A, S73Y, N79T, and N96H (TadA-CD-21, SEQ ID NO: 3622) relative to the amino acid sequence of SEQ ID NO: 3600. In some embodiments, the evolved TadA-Dual deaminase comprises the mutations N461, S73P, and N79T (TadA-CD-22, SEQ ID NO: 3623) relative to the amino acid sequence of SEQ ID NO: 3600. In some embodiments, the evolved TadA-Dual deaminase comprises the mutations N46V, S73P, N79T, and G106S (TadA-CD-23, SEQ ID NO: 3624) relative to the amino acid sequence of SEQ ID NO: 3600. In some embodiments, the evolved TadA-Dual deaminase comprises the mutations R48P, S73H, and N79P (TadA-CD-24, SEQ ID NO: 3625) relative to the amino acid sequence of SEQ ID NO: 3600. In some embodiments, the evolved TadA-Dual deaminase comprises the mutations A28V, N46I, R48A, S73Y, and N79T (TadA-CD-25, SEQ ID NO: 3626) relative to the amino acid sequence of SEQ ID NO: 3600.

In some embodiments, the evolved TadA-Dual deaminase comprises the mutations N46V and S73P (TadA-CD-26, SEQ ID NO: 3627) relative to the amino acid sequence of SEQ ID NO: 3600. In some embodiments, the evolved TadA-Dual deaminase comprises the mutation N46L (TadA-CD-27, SEQ ID NO: 3628) relative to the amino acid sequence of SEQ ID NO: 3600. In some embodiments, the evolved TadA-Dual deaminase comprises the mutations N46C, S73Y, and A162V (TadA-CD-28, SEQ ID NO: 3629) relative to the amino acid sequence of SEQ ID NO: 3600. In some embodiments, the evolved TadA-Dual deaminase comprises the mutations N46V, Q71H, and S73P (TadA-CD-29, SEQ ID NO: 3630) relative to the amino acid sequence of SEQ ID NO: 3600. In some embodiments, the evolved TadA-Dual deaminase comprises the mutations N46C and S73P (TadA-CD-30, SEQ ID NO: 3631) relative to the amino acid sequence of SEQ ID NO: 3600. In some embodiments, the evolved TadA-Dual deaminase comprises the mutations N46C, S73P, and A162V (TadA-CD-31, SEQ ID NO: 3632) relative to the amino acid sequence of SEQ ID NO: 3600. In some embodiments, the evolved TadA-Dual deaminase comprises the mutations N46V and S73P (TadA-CD-32, SEQ ID NO: 3633) relative to the amino acid sequence of SEQ ID NO: 3600. In some embodiments, the evolved TadA-Dual deaminase comprises the mutation N46V (TadA-CD-33, SEQ ID NO: 3634) relative to the amino acid sequence of SEQ ID NO: 3600. In some embodiments, the evolved TadA-Dual deaminase comprises the mutations N46V and R48P (TadA-CD-34, SEQ ID NO: 3635) relative to the amino acid sequence of SEQ ID NO: 3600. In some embodiments, the evolved TadA-Dual deaminase comprises the mutations N46CV and S73P (TadA-CD-35, SEQ ID NO: 3636) relative to the amino acid sequence of SEQ ID NO: 3600. In some embodiments, the evolved TadA-Dual deaminase comprises the mutations L34M, N46L and S73P (TadA-CD-36, SEQ ID NO: 3637) relative to the amino acid sequence of SEQ ID NO: 3600. In some embodiments, the evolved TadA-Dual deaminase comprises the mutations N46L and S73P (TadA-CD-37, SEQ ID NO: 3638) relative to the amino acid sequence of SEQ ID NO: 3600. In some embodiments, the evolved TadA-Dual deaminase comprises the mutations N46L, r48P, R64K and S73P (TadA-CD-38, SEQ ID NO: 3639) relative to the amino acid sequence of SEQ ID NO: 3600.

In some embodiments, the TadA-CDs evolved from TadA-dual comprise at least 80%, 85%, 90%, 95%, 98%, 99%, or 99.5% identical to any of the amino acid sequences of SEQ ID NOs: 39, 41-54, and 359-383.

Exemplary TadA-derived cytosine base editor amino acid sequences include: TadA-CDa base editor (SpCas9n napDNAbp domain) (TadCBEa) (SEQ ID NO: 3640), TadA-CDb base editor (SpCas9n napDNAbp domain) (TadCBEb) (SEQ ID NO: 3641), TadA-CDc base editor (SpCas9n napDNAbp domain) (TadCBEc) (SEQ ID NO: 3642), TadA-CDd base editor (SpCas9n napDNAbp domain) (TadCBEd) (SEQ ID NO: 3643), TadA-CDe base editor (SpCas9n napDNAbp domain) (TadCBEe) (SEQ ID NO: 3644), TadA-CDa (V106W) base editor (SpCas9n napDNAbp domain) (TadCBEa (V106W)) (SEQ ID NO: 3645), TadA-CDd (V106W) base editor (SpCas9n napDNAbp domain) (TadCBEd (V106W)) (SEQ ID NO: 3646), TadA-CDf base editor (SpCas9n napDNAbp domain) (TadCBEf) (SEQ ID NO: 3647), TadA-CDg base editor (SpCas9n napDNAbp domain) (TadCBEg) (SEQ ID NO: 3648), TadA-CDa: eNme2Cas9 base editor (SEQ ID NO: 3649), TadA-CDa: SaCas9 base editor (SEQ ID NO: 3650), TadA-CDa: SpCas9-NG base editor (SEQ ID NO: 3651), TadA-CDa: enCjCas9 base editor (SEQ ID NO: 3652).

Exemplary polynucleotides encoding TadA-derived cytosine base editors of the disclosure include: TadCBEa-eNme2-C-BE4max vector (SEQ ID NO: 3653), TadCBEa-enCjCas9-BE4max vector (SEQ ID NO: 3654), TadCBEa-SpCas9-BE4max vector (SEQ ID NO: 3655), TadCBEa-SaCas9-BE4max vector (SEQ ID NO: 3656), TadCBEa-SpCas9-NG-BE4max vector (SEQ ID NO: 3657).

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

In an embodiment, a guide polynucleotide described herein can be RNA or DNA. In one embodiment, the guide polynucleotide is a gRNA.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide can comprise a CRISPR RNA (crRNA) and a trans-activating CRISPR RNA (tracrRNA) or can comprise one or more trans-activating CRISPR RNA (tracrRNA).

A guide polynucleotide may include natural or non-natural (or unnatural) nucleotides (e.g., peptide nucleic acid or nucleotide analogs). In some cases, the targeting region of a guide nucleic acid sequence (e.g., a spacer) can be at least 15, 16, 17, 18, 19, 20, 21, 22, 23, 24, 25, 26, 27, 28, 29, or 30 nucleotides in length.

In some embodiments, the methods described herein can utilize an engineered Cas protein. A guide RNA (gRNA) is a short synthetic RNA composed of a scaffold sequence necessary for Cas-binding and a user-defined ~20 nucleotide spacer that defines the genomic target to be modified. Exemplary gRNA scaffold sequences are provided in the sequence listing as SEQ ID NOs: 317-327 and 425. Thus, a skilled artisan can change the genomic target of the Cas protein specificity is partially determined by how specific the gRNA targeting sequence is for the genomic target compared to the rest of the genome. In embodiments, the spacer is about 10, 11, 12, 13, 14, 15, 16, 17, 18, 19, 20, 22, 23, 24, 25, or more nucleotides in length. The spacer of a gRNA can be or can be about 19, 20, or 21 nucleotides in length.

A gRNA or a guide polynucleotide can target any exon or intron of a gene target. In some embodiments, a composition comprises multiple gRNAs that all target the same exon or multiple gRNAs that target different exons. An exon and/or an intron of a gene can be targeted. A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

The guide polynucleotides can comprise standard ribonucleotides, modified ribonucleotides (e.g., pseudouridine), ribonucleotide isomers, and/or ribonucleotide analogs.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and may be separated by a direct repeat.

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., N1-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length spacer. In some embodiments, the guide comprises a 20-40 nucleotide spacer. In some embodiments, the guide comprises a spacer comprising at least about 20-25 nucleotides or at least about 30-35 nucleotides. In some embodiments, the spacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length spacer; and
a spacer comprising modified nucleotides.

In embodiments, the gRNA contains numerous modified nucleotides and/or chemical modifications. Such modifications can increase base editing ~2 fold in vivo or in vitro. In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold.

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation. In some cases, phosphorothioate bonds can be added throughout an entire gRNA to reduce attack by endonucleases.

### Fusion Proteins or Complexes Comprising a Nuclear Localization Sequence (NLS)

In some embodiments, the fusion proteins or complexes provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas 12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences.

In some embodiments, the NLS is present in a linker or the NLS is flanked by linkers, for example described herein. A bipartite NLS comprises two basic amino acid clusters, which are separated by a relatively short spacer sequence (hence bipartite-2 parts, while monopartite NLSs are not). The NLS of nucleoplasmin, KR [PAATKKAGQA]KKKK (SEQ ID NO: 191), is the prototype of the ubiquitous bipartite signal: two clusters of basic amino acids, separated by a spacer of about 10 amino acids. The sequence of an exemplary bipartite NLS follows:

(SEQ ID NO: 328)
PKKKRKVEGADKRTADGSEFESPKKKRKV.

In some embodiments, any of the fusion proteins or complexes provided herein comprise an NLS comprising the amino acid sequence EGADKRTADGSEFESPKKKRKV (amino acids 8 to 29 of SEQ ID NO 328). In some embodiments, any of the adenosine base editors provided herein comprise an NLS comprising the amino acid sequence EGADKRTADGSEFESPKKKRKV (amino acids 8 to 29 of SEQ ID NO: 328). In some embodiments, the NLS is at a C-terminal portion of the adenosine base editor. In some embodiments, the NLS is at the C-terminus of the adenosine base editor.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor comprises a nuclease, a nickase, a recombinase, a deaminase, a methyltransferase, a methylase, an acetylase, an acetyltransferase, a transcriptional activator, or a transcriptional repressor domain.

In some embodiments, a base editor comprises an uracil glycosylase inhibitor (UGI) domain. In some cases, a base editor is expressed in a cell in trans with a UGI polypeptide. In some embodiments, cellular DNA repair response to the presence of U:G heteroduplex DNA can be responsible for a reduction in nucleobase editing efficiency in cells. In such embodiments, uracil DNA glycosylase (UDG) can catalyze removal of U from DNA in cells, which can initiate base excision repair (BER), mostly resulting in reversion of the U:G pair to a C:G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein or complex comprising a UGI domain and/or a uracil stabilizing protein (USP) domain.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is a cytidine base editor (CBE) or an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be a cytidine deaminase or an cytosine deaminase. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleotide (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

The components of a base editor system (e.g., a deaminase domain, a guide RNA, and/or a polynucleotide programmable nucleotide binding domain) may be associated with each other covalently or non-covalently. For example, in some embodiments, the deaminase domain can be targeted to a target nucleotide sequence by a polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component (e.g., the deaminase component) comprises an additional heterologous portion or domain that is capable of interacting with, associating with, or capable of forming a complex with a corresponding heterologous portion, antigen, or domain that is part of a polynucleotide programmable nucleotide binding domain and/or a guide polynucleotide (e.g., a guide RNA) complexed therewith. In some embodiments, the polynucleotide programmable nucleotide binding domain, and/or a guide polynucleotide (e.g., a guide RNA) complexed therewith, comprises an additional heterologous portion or domain that is capable of interacting with, associating with, or capable of forming a complex with a corresponding heterologous portion, antigen, or domain that is part of a nucleobase editing domain (e.g., the deaminase component). In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion is capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH₂) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fc domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g., an MS2, an MS2 C-5 mutant, or an MS2 F-5 mutant), a non-natural RNA motif, a PP7 operator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some instances, components of the base editing system are associated with one another through the interaction of leucine zipper domains (e.g., SEQ ID NOs: 387 and 388). In some cases, components of the base editing system are associated with one another through polypeptide domains (e.g., FokI domains) that associate to form protein complexes containing about, at least about, or no more than about 1, 2 (i.e., dimerize), 3, 4, 5, 6, 7, 8, 9, 10 polypeptide domain units, optionally the polypeptide domains may include alterations that reduce or eliminate an activity thereof.

In some instances, components of the base editing system are associated with one another through the interaction of multimeric antibodies or fragments thereof (e.g., IgG, IgD, IgA, IgM, IgE, a heavy chain domain 2 (CH₂) of IgM (MHD2) or IgE (EHD2), an immunoglobulin Fc region, a heavy chain domain 3 (CH3) of IgG or IgA, a heavy chain domain 4 (CH4) of IgM or IgE, an Fab, and an Fab2). In some instances, the antibodies are dimeric, trimeric, or tetrameric. In embodiments, the dimeric antibodies bind a polypeptide or polynucleotide component of the base editing system.

In some cases, components of the base editing system are associated with one another through the interaction of a polynucleotide-binding protein domain(s) with a polynucleotide(s). In some instances, components of the base editing system are associated with one another through the interaction of one or more polynucleotide-binding protein domains with polynucleotides that are self-complementary and/or complementary to one another so that complementary binding of the polynucleotides to one another brings into association their respective bound polynucleotide-binding protein domain(s).

In some instances, components of the base editing system are associated with one another through the interaction of a polypeptide domain(s) with a small molecule(s) (e.g., chemical inducers of dimerization (CIDs), also known as “dimerizers”). Non-limiting examples of CIDs include those disclosed in Amara, et al., “A versatile synthetic dimerizer for the regulation of protein-protein interactions,” PNAS, 94:10618-10623 (1997); and Voß, et al. “Chemically induced dimerization: reversible and spatiotemporal control of protein function in cells,” Current Opinion in Chemical Biology, 28:194-201 (2015), the disclosures of each of which are incorporated herein by reference in their entireties for all purposes. In some embodiments, the base editor inhibits base excision repair (BER) of the edited strand. In some embodiments, the base editor protects or binds the non-edited strand. In some embodiments, the base editor comprises UGI activity or USP activity. In some embodiments, the base editor comprises a catalytically inactive inosine-specific nuclease.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Protein domains included in the fusion protein can be a heterologous functional domain. Non-limiting examples of protein domains which can be included in the fusion protein include a deaminase domain (e.g., cytidine deaminase and/or adenosine deaminase), a uracil glycosylase inhibitor (UGI) domain, epitope tags, and reporter gene sequences. In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises an evolved TadA variant. In some embodiments, the base editor is ABE8.1, which comprises or consists essentially of the following sequence or a fragment thereof having adenosine deaminase activity: SEQ ID NO: 331. Other ABE8 sequences are provided in the attached sequence listing (SEQ ID NOs: 332-354).

In some embodiments, the base editor includes an adenosine deaminase variant comprising an amino acid sequence, which contains alterations relative to an ABE 7*10 reference sequence, as described herein. The term “monomer” as used in Table 7 refers to a monomeric form of TadA*7.10 comprising the alterations described. The term “heterodimer” as used in Table 7 refers to the specified wild-type E. coli TadA adenosine deaminase fused to a TadA*7.10 comprising the alterations as described.

TABLE 7
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + Q154S |
| ABE-680m | MSP680 | monomer_TadA*7.10 + I76Y + V82G + Y147T + Q154S |
| ABE-823m | MSP823 | monomer_TadA*7.10 + L36H + V82G + Y147T + Q154S + |
| | | N157K |
| ABE-824m | MSP824 | monomer_TadA*7.10 + V82G + Y147D + F149Y + Q154S + |
| | | D167N |
| ABE-825m | MSP825 | monomer_TadA*7.10 + L36H + V82G + Y147D + F149Y + |
| | | Q154S + N157K + D167N |
| ABE-827m | MSP827 | monomer_TadA*7.10 + L36H + I76Y + V82G + Y147T + Q154S + |
| | | N157K |
| ABE-828m | MSP828 | monomer_TadA*7.10 + I76Y + V82G + Y147D + F149Y + Q154S + |
| | | D167N |
| ABE-829m | MSP829 | monomer_TadA*7.10 + L36H + I76Y + V82G + Y147D + F149Y + |
| | | Q154S + N157K + D167N |
| ABE-605d | MSP605 | heterodimer_(WT) + (TadA*7.10 + V82G + Y147T + Q154S) |
| ABE-680d | MSP680 | heterodimer_(WT) + (TadA*7.10 + I76Y + V82G + Y147T + |
| | | Q154S) |
| ABE-823d | MSP823 | heterodimer_(WT) + (TadA*7.10 + L36H + V82G + Y147T + |
| | | Q154S + N157K) |
| ABE-824d | MSP824 | heterodimer_(WT) + (TadA*7.10 + V82G + Y147D + F149Y + |
| | | Q154S + D167N) |
| ABE-825d | MSP825 | heterodimer_(WT) + (TadA*7.10+ L36H + V82G + Y147D + |
| | | F149Y + Q154S + N157K + D167N) |
| ABE-827d | MSP827 | heterodimer_(WT) + (TadA*7.10 + L36H + I76Y + V82G + Y147T + |
| | | Q154S + N157K) |
| ABE-828d | MSP828 | heterodimer_(WT) + (TadA*7.10 + I76Y + V82G + Y147D + |
| | | F149Y + Q154S + D167N) |
| ABE-829d | MSP829 | heterodimer_(WT) + (TadA*7.10+ L36H + I76Y + V82G + Y147D + |
| | | F149Y + Q154S + N157K + D167N) |

In some embodiments, the base editor comprises a domain comprising all or a portion (e.g., a functional portion) of a uracil glycosylase inhibitor (UGI) or a uracil stabilizing protein (USP) domain.

### Linkers

In certain embodiments, linkers may be used to link any of the peptides or peptide domains of the disclosure. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.).

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS)ₙ (SEQ ID NO: 246), (GGGGS)ₙ (SEQ ID NO: 247), and (G)n to more rigid linkers of the form (EAAAK)ₙ (SEQ ID NO: 248), (SGGS)ₙ (SEQ ID NO: 355), SGSETPGTSESATPES (SEQ ID NO: 249) (see, e.g., Guilinger J P, et al. Fusion of catalytically inactive Cas9 to FokI nuclease improves the specificity of genome modification. Nat. Biotechnol. 2014; 32 (6): 577-82; the entire contents are incorporated herein by reference) and (XP) n) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS) n motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES (SEQ ID NO: 249), which can also be referred to as the XTEN linker.

In some embodiments, the domains of the base editor are fused via a linker that comprises the amino acid sequence of:

(SEQ ID NO: 356)
SGGSSGSETPGTSESATPESSGGS,
(SEQ ID NO: 357)
SGGSSGGSSGSETPGTSESATPESSGGSSGGS,
(SEQ ID NO: 358)
GGSGGSPGSPAGSPTSTEEGTSESATPESGPGTSTEPSEGSAPGSPAGS
PTSTEEGTSTEPSEGSAPGTSTEPSEGSAPGTSESATPESGPGSEPATS
GGSGGS,
(SEQ ID NO: 3573)
EGGSEEEEESGS,
or
(SEQ ID NO: 3574)
KGPKPKKEESEK.

In some embodiments, domains of the base editor are fused via a linker comprising the amino acid sequence SGSETPGTSESATPES (SEQ ID NO: 249), which may also be referred to as the XTEN linker. In some embodiments, a linker comprises the amino acid sequence SGGS (SEQ ID NO: 355). In some embodiments, the linker is 24 amino acids in length. In some embodiments, the linker comprises the amino acid sequence SGGSSGGSSGSETPGTSESATPES (SEQ ID NO: 359). In some embodiments, the linker is 40 amino acids in length. In some embodiments, the linker comprises the amino acid sequence: SGGSSGGSSGSETPGTSESATPESSGGSSGGSSGGSSGGS (SEQ ID NO: 360). In some embodiments, the linker is 64 amino acids in length. In some embodiments, the linker comprises the amino acid sequence: SGGSSGGSSGSETPGTSESATPESSGGSSGGSSGGSSGGSSGSETPGTSESATPESSGGSSG GS (SEQ ID NO: 361). In some embodiments, the linker is 92 amino acids in length. In some embodiments, the linker comprises the amino acid sequence:

(SEQ ID NO: 362)
PGSPAGSPTSTEEGTSESATPESGPGTSTEPSEGSAPGSPAGSPTSTEE
GTSTEPSEGSAPGTSTEPSEGSAPGTSESATPESGPGSEPATS.

In some embodiments, a linker comprises a plurality of proline residues and is 5-21, 5-14, 5-9, 5-7 amino acids in length, e.g., PAPAP (SEQ ID NO: 363), PAPAPA (SEQ ID NO: 364), PAPAPAP (SEQ ID NO: 365), PAPAPAPA (SEQ ID NO: 366), P (AP) 4 (SEQ ID NO: 367), P (AP) 7 (SEQ ID NO: 368), P (AP) 10 (SEQ ID NO: 369) (see, e.g., Tan J, Zhang F, Karcher D, Bock R. Engineering of high-precision base editors for site-specific single nucleotide replacement. Nat Commun. 2019 Jan. 25; 10 (1): 439; the entire contents are incorporated herein by reference). Such proline-rich linkers are also termed “rigid” linkers.

In various embodiments, a linker of the disclosure comprises 8, 9, 10, 11, 12, 13, 14, or 15 amino acids. In some cases, the linker comprises a sequence selected from one or more of EGSSSKEEEEPG (SEQ ID NO: 647), NSISSSNGOK (SEQ ID NO: 648), GEEGEGSGGGEK (SEQ ID NO: 649), EGEGGKESGSSE (SEQ ID NO: 650), GGGGSSKSPGSE (SEQ ID NO: 651), PIGSDODD (SEQ ID NO: 652), TEKGQVPHGS (SEQ ID NO: 653), ESGEGGGGSEKK (SEQ ID NO: 654), EEGKPKEGEGSG (SEQ ID NO: 655), ASREPKDSS (SEQ ID NO: 656), KOGSEHDE (SEQ ID NO: 657), SESKSEKGSSEK (SEQ ID NO: 658), QYDSGERSDQ (SEQ ID NO: 659), PGANEEIPGQ (SEQ ID NO: 660), NSPTDEK (SEQ ID NO: 661), EGANEEIPGQ (SEQ ID NO: 662), EGEKEKKKSGES (SEQ ID NO: 663), PGRHEEVPGQ (SEQ ID NO: 664), SKHQTEQDDS (SEQ ID NO: 665), ESEDDSSGRK (SEQ ID NO: 666), KESEKKESESKS (SEQ ID NO: 667), KGEGKSSIKD (SEQ ID NO: 668), DRSQKQDQQD (SEQ ID NO: 669), GPSSTSSS (SEQ ID NO: 670), GSSGEKEEGEPS (SEQ ID NO: 671), GEPKSKKSGSGS (SEQ ID NO: 672), SSGEGGKSESGP (SEQ ID NO: 673), SPQPTSSD (SEQ ID NO: 674), EGGSEEEEESGS (SEQ ID NO: 675), KGPKPKKEESEK (SEQ ID NO: 676), SKSQQFVTYE (SEQ ID NO: 677), TGNSKYQTGK (SEQ ID NO: 678), PQPIPHTNPT (SEQ ID NO: 679), ANAHSDISTG (SEQ ID NO: 680), KSQQTEDQSK (SEQ ID NO: 681), QSQDQKQKEH (SEQ ID NO: 682), NQQRPSSD (SEQ ID NO: 683), TTKDTSPKPQ (SEQ ID NO: 684), EGKDNOQTGE (SEQ ID NO: 685), or EPQPDSSE (SEQ ID NO: 686).

Nucleic Acid Programmable DNA Binding Proteins with Guide RNAs

Provided herein are compositions and methods for base editing in cells. Further provided herein are compositions comprising a guide polynucleotide sequence, e.g., a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g., a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in a cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide systems comprising any of the fusion proteins or complexes provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein or complex. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 3 or 5′-NAA-3′). In some embodiments, the guide nucleic acid (e.g., guide RNA) is complementary to a sequence in a gene of interest (e.g., a gene associated with a disease or disorder).

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA, wherein the guide RNA is about 15-100 nucleotides long and comprises a sequence of at least 10 contiguous nucleotides that is complementary to a target sequence.

The domains of the base editor disclosed herein can be arranged in any order.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence.

### Methods of Using Fusion Proteins or Complexes Comprising a Cytidine or Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA described herein.

In some embodiments, a fusion protein or complex of the disclosure is used for editing a target gene of interest. In particular, a cytidine deaminase or adenosine deaminase nucleobase editor described herein is capable of making multiple mutations within a target sequence. These mutations may affect the function of the target. For example, when a cytidine deaminase or adenosine deaminase nucleobase editor is used to target a regulatory region the function of the regulatory region is altered and the expression of the downstream protein is reduced or eliminated.

### Base Editor Efficiency

In some embodiments, the purpose of the methods provided herein is to alter a gene and/or gene product via gene editing. The nucleobase editing proteins provided herein can be used for gene editing-based human therapeutics in vitro or in vivo. It will be understood by the skilled artisan that the nucleobase editing proteins provided herein, e.g., the fusion proteins or complexes comprising a polynucleotide programmable nucleotide binding domain (e.g., Cas9) and a nucleobase editing domain (e.g., an adenosine deaminase domain or a cytidine deaminase domain) can be used to edit a nucleotide from A to G or C to T.

Advantageously, base editing systems as provided herein provide genome editing without generating double-strand DNA breaks, without requiring a donor DNA template, and without inducing an excess of stochastic insertions and deletions as CRISPR may do. In some embodiments, the present disclosure provides base editors that efficiently generate an intended mutation, such as a STOP codon, in a nucleic acid (e.g., a nucleic acid within a genome of a subject) without generating a significant number of unintended mutations, such as unintended point mutations.

The base editors of the disclosure advantageously modify a specific nucleotide base encoding a protein without generating a significant proportion of indels (i.e., insertions or deletions). Such indels can lead to frame shift mutations within a coding region of a gene.

In some embodiments, the base editors provided herein are capable of generating a ratio of intended mutations to indels (i.e., intended point mutations: unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%.

Base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence and may affect the gene product.

In some embodiments, the modification, e.g., single base edit results in about or at least about a 10%, 15%, 20%, 25%, 30%, 35%, 40%, 45%, 50%, 55%, 60%, 65%, 70%, 75%, 80%, 85%, 90%, 95%, 99%, or 100% reduction, or reduction to an undetectable level, of the gene targeted expression.

The disclosure provides adenosine deaminase variants (e.g., ABE8 variants) that have increased efficiency and specificity. In particular, the adenosine deaminase variants described herein are more likely to edit a desired base within a polynucleotide and are less likely to edit bases that are not intended to be altered (e.g., “bystanders”).

In some embodiments, any of the base editing system comprising one of the ABE8 base editor variants described herein has reduced bystander editing or mutations by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 99% compared to a base editor system comprising an ABE7 base editor, e.g., ABE7.10.

In some embodiments, any of the ABE8 base editor variants described herein has higher base editing efficiency compared to the ABE7 base editors. In some embodiments, any of the ABE8 base editor variants described herein have at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, 100%, 105%, 110%, 115%, 120%, 125%, 130%, 135%, 140%, 145%, 150%, 155%, 160%, 165%, 170%, 175%, 180%, 185%, 190%, 195%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, or 500% higher base editing efficiency compared to an ABE7 base editor, e.g., ABE7.10.

The ABE8 base editor variants described herein may be delivered to a host cell via a plasmid, a vector, a LNP complex, or an mRNA. In some embodiments, any of the ABE8 base editor variants described herein is delivered to a host cell as an mRNA.

In some embodiments, the method described herein, for example, the base editing methods has minimum to no off-target effects. In some embodiments, the method described herein, for example, the base editing methods, has minimal to no chromosomal translocations.

In some embodiments, the base editing method described herein results in about 91%, 92%, 93%, 94%, 95%, 96%, 97%, 98%, 99% or 100% of a cell population that have been successfully edited.

In some embodiments, the percent of viable cells in a cell population following a base editing intervention is greater than at least 60%, 70%, 80%, or 90% of the starting cell population at the time of the base editing event. In some embodiments, the percent of viable cells in a cell population following editing is about 70%. In some embodiments, the percent of viable cells in a cell population following editing is about 75%. In some embodiments, the percent of viable cells in a cell population following editing is about 80%. In some embodiments, the percent of viable cells in a cell population as described above is about 85%. In some embodiments, the percent of viable cells in a cell population as described above is about 90%, or about 91%, 92%, 93%, 94% 95%, 96%, 97%, 98%, 99%, or 100% of the cells in the population at the time of the base editing event.

In embodiments, the cell population is a population of cells contacted with a base editor, complex, or base editor system of the present disclosure.

The number of intended mutations and indels can be determined using any suitable method, for example, as described in International PCT Application Nos. PCT/US2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T: A base editors with higher efficiency and product purity” Science Advances 3: eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

### Multiplex Editing

In some embodiments, the base editor system provided herein is capable of multiplex editing of a plurality of nucleobase pairs in one or more genes or polynucleotide sequences. In some embodiments, the plurality of nucleobase pairs is located in the same gene or in one or more genes, wherein at least one gene is located in a different locus. In some embodiments, the multiplex editing comprises one or more guide polynucleotides. In some embodiments, the multiplex editing comprises one or more base editor systems. In some embodiments, the multiplex editing comprises one or more base editor systems with a single guide polynucleotide or a plurality of guide polynucleotides. In some embodiments, the multiplex editing comprises one or more guide polynucleotides with a single base editor system. It should be appreciated that the characteristics of the multiplex editing using any of the base editors as described herein can be applied to any combination of methods using any base editor provided herein. It should also be appreciated that the multiplex editing using any of the base editors as described herein can comprise a sequential editing of a plurality of nucleobase pairs.

In some embodiments, the base editor system capable of multiplex editing of a plurality of nucleobase pairs in one or more genes comprises one of ABE7, ABE8, and/or ABE9 base editors.

### Expression of Fusion Proteins or Complexes in a Host Cell

Fusion proteins or complexes of the disclosure comprising a deaminase may be expressed in virtually any host cell of interest, including but not limited to bacteria, yeast, fungi, insects, plants, and animal cells using routine methods known to the skilled artisan. For example, a DNA encoding an adenosine deaminase of the disclosure can be cloned by designing suitable primers for the upstream and downstream of CDS based on the cDNA sequence. The cloned DNA may be directly, or after digestion with a restriction enzyme when desired, or after addition of a suitable linker and/or a nuclear localization signal, ligated with a DNA encoding one or more additional components of a base editing system. The base editing system is translated in a host cell to form a complex.

A polynucleotide encoding a polypeptide described herein can be obtained by chemically synthesizing the polynucleotide, or by connecting synthesized partly overlapping oligo short chains by utilizing the PCR method and the Gibson Assembly method to construct a polynucleotide (e.g., DNA) encoding the full length thereof. The advantage of constructing a full-length polynucleotide by chemical synthesis or a combination of PCR method or Gibson Assembly method is that the codons to be used can be selected in according to the host into which the polynucleotide is to be introduced. In the expression from a heterologous DNA molecule, the protein expression level is expected to increase by converting the DNA sequence thereof to a codon highly frequently used in the host organism. Codon use data for a host cell (e.g., codon use data available at kazusa.or.jp/codon/index.html) can be used to guide codon optimization for a polynucleotide sequence encoding a polypeptide. Codons having low use frequency in the host may be converted to a codon coding the same amino acid and having high use frequency.

An expression vector containing a polynucleotide encoding a nucleic acid sequence-recognizing module and/or a nucleic acid base converting enzyme can be produced, for example, by linking the DNA to the downstream of a promoter in a suitable expression vector.

As the expression vector, Escherichia coli-derived plasmids (e.g., pBR322, pBR325, pUC12, pUC13); Bacillus subtilis-derived plasmids (e.g., pUB110, pTP5, pC194); yeast-derived plasmids (e.g., pSH19, pSH15); insect cell expression plasmids (e.g., pFast-Bac); animal cell expression plasmids (e.g., pA1-11, pXT1, pRc/CMV, pRc/RSV, pcDNAI/Neo); bacteriophages such as .lambda phage and the like; insect virus vectors such as baculovirus and the like (e.g., BmNPV, AcNPV); animal virus vectors such as retrovirus, vaccinia virus, adenovirus and the like, and the like are used.

Regarding the promoter to be used, any promoter appropriate for a host to be used for gene expression can be used. In a method using double-stranded breaks, since the survival rate of the host cell sometimes reduces markedly due to the toxicity, it is desirable to increase the number of cells by the start of the induction by using an inductive promoter. However, since sufficient cell proliferation can also be afforded by expressing the nucleic acid-modifying enzyme complex of the present disclosure, a constitutive promoter can be used without limitation.

For example, when the host is an animal cell, an SR.alpha. promoter, SV40 promoter, LTR promoter, cytomegalovirus (CMV) promoter, Rous sarcoma virus (RSV) promoter, Moloney mouse leukemia virus (MoMuLV), LTR, herpes simplex virus thymidine kinase (HSV-TK) promoter, and the like can be used. Of these, CMV promoter, SR.alpha. promoter and the like may be used.

Expression vectors for use in the present disclosure, besides those mentioned above, can comprise an enhancer, a splicing signal, a terminator, a poly A addition signal, a selection marker such as drug resistance gene, an auxotrophic complementary gene and the like, a replication origin, and the like can be used.

An RNA encoding a protein domain described herein can be prepared by, for example, in vitro transcription of a nucleic acid sequence encoding any of the fusion proteins or complexes disclosed herein.

A fusion protein or complex of the disclosure can be intracellularly expressed by introducing into the cell an expression vector comprising a nucleic acid sequence encoding the fusion protein or complex.

An expression vector can be introduced by a known method (e.g., the lysozyme method, the competent method, the PEG method, the CaCl₂) coprecipitation method, electroporation, microinjection, particle gun method, lipofection, Agrobacterium-mediated delivery, etc.) according to the kind of the host.

A vector can be introduced into an animal cell according to the methods described in, for example, Cell Engineering additional volume 8, New Cell Engineering Experiment Protocol, 263-267 (1995) (published by Shujunsha), and Virology, 52, 456 (1973).

A cell comprising a vector can be cultured according to a known method according to the kind of the host.

As a medium for culturing an animal cell, for example, minimum essential medium (MEM) containing about 5 to about 20% of fetal bovine serum [Science, 122, 501 (1952)], Dulbecco's modified Eagle medium (DMEM) [Virology, 8, 396 (1959)], RPMI 1640 medium [The Journal of the American Medical Association, 199, 519 (1967)], 199 medium [Proceeding of the Society for the Biological Medicine, 73, 1 (1950)] and the like are used. The pH of the medium may be between about 6 to about 8. The culture is performed at generally about 30° C. to about 40° C. Where necessary, aeration and stirring may be performed.

When a higher eukaryotic cell, such as animal cell, insect cell, plant cell and the like is used as a host cell, a polynucleotide encoding a base editing system of the present disclosure (e.g., comprising an adenosine deaminase variant) is introduced into a host cell under the regulation of an inducible promoter (e.g., metallothionein promoter (induced by heavy metal ion), heat shock protein promoter (induced by heat shock), Tet-ON/Tet-OFF system promoter (induced by addition or removal of tetracycline or a derivative thereof), steroid-responsive promoter (induced by steroid hormone or a derivative thereof) etc.), the induction substance is added to the medium (or removed from the medium) at an appropriate stage to induce expression of the nucleic acid-modifying enzyme complex, culture is performed for a given period to carry out a base editing and, introduction of a mutation into a target gene, transient expression of the base editing system can be realized.

Alternatively, an inductive promoter can also be utilized as a vector removal mechanism when higher eukaryotic cells, such as animal cell, insect cell, plant cell and the like are used as a host cell. That is, a vector is mounted with a replication origin that functions in a host cell, and a nucleic acid encoding a protein necessary for replication (e.g., SV40 on and large T antigen, oriP and EBNA-1 etc. for animal cells), of the expression of the nucleic acid encoding the protein is regulated by the above-mentioned inducible promoter. As a result, while the vector is autonomously replicable in the presence of an induction substance, when the induction substance is removed, autonomous replication is not available, and the vector naturally falls off along with cell division (autonomous replication is not possible by the addition of tetracycline and doxycycline in Tet-OFF system vector).

### Reducing Expression of Target Genes in Cells

In some embodiments, provided herein is a cell (e.g., cell from the liver, eye, and/or a central nervous system or component thereof) with at least one modification in an endogenous gene or one or more regulatory elements thereof. Provided herein are also methods, base editors, base editor systems, guide RNAs, and compositions for modifying the cell. In some embodiments, the cell may comprise a further modification in at least one, two, three, four, five, six, seven, eight, nine, ten, eleven, twelve, thirteen, fourteen, fifteen, sixteen, seventeen, eighteen, nineteen, twenty or more endogenous genes or regulatory elements thereof. In some embodiments, the at least one modification is a single nucleobase modification. In some embodiments, the at least one modification is generated by base editing. The base editing may be positioned at any suitable position of the gene, or in a regulatory element of the gene. Thus, it may be appreciated that a single base editing at a start codon, for example, can completely abolish the expression of the gene. In some embodiments, the base editing may be performed at a site within an exon. In some embodiments, the base editing may be performed at a site on more than one exons. In some embodiments, the base editing may be performed at any exon of the multiple exons in a gene. In some embodiments, base editing may introduce a premature STOP codon into an exon, resulting in either lack of a translated product or in a truncated that may be misfolded and thereby eliminated by degradation, or may produce an unstable mRNA that is readily degraded. In some embodiments, the cell is a hepatocyte, and/or a cell from the liver, eye, and/or a central nervous system or component thereof.

In some embodiments, the gene is a factor B polynucleotide.

In some embodiments, the editing of the endogenous gene reduces expression of the gene. In some embodiments, the editing of the endogenous gene reduces expression of the gene by at least 50% as compared to a control cell without the modification. In some embodiments, the editing of the endogenous gene reduces expression of the gene by at least 60% as compared to a control cell without the modification. In some embodiments, the editing of the endogenous gene reduces expression of the gene by at least 70% as compared to a control cell without the modification. In some embodiments, the editing of the endogenous gene reduces expression of the gene by at least 80% as compared to a control cell without the modification. In some embodiments, the editing of the endogenous gene reduces expression of the gene by at least 90% as compared to a control cell without the modification. In some embodiments, the editing of the endogenous gene reduces expression of the gene by at least 100% as compared to a control cell without the modification. In some embodiments, the editing of the endogenous gene eliminates gene expression.

In some embodiments, base editing may be performed on an intron. For example, base editing may be performed on an intron. In some embodiments, the base editing may be performed at a site within an intron. In some embodiments, the base editing may be performed at sites in one or more introns. In some embodiments, the base editing may be performed at any intron of the multiple introns in a gene. In some embodiments, one or more base edits may be performed on an exon, an intron, or any combination of exons and introns.

In some embodiments, the modification or base edit may be within a promoter site. In some embodiments, the base edit may be introduced within an alternative promoter site. In some embodiments, the base edit may be in a 5′ regulatory element, such as an enhancer. In some embodiment, base editing may be introduced to disrupt the binding site of a nucleic acid binding protein. Exemplary nucleic acid binding proteins may be a polymerase, nuclease, gyrase, topoisomerase, methylase or methyl transferase, transcription factors, enhancer, PABP, zinc finger proteins, among many others.

In some embodiments, base editing may be used for splice disruption to silence target protein expression. In some embodiments, base editing may generate a splice acceptor-splice donor (SA-SD) site. Targeted base editing generating a SA-SD, or at a SA-SD site can result in reduced expression of a gene. In some embodiments, base editors (e.g., ABE, CBE, or CABE) are used to target dinucleotide motifs that constitute splice acceptor and splice donor sites, which are the first and last two nucleotides of each intron. In some embodiments, splice disruption is achieved with an adenosine base editor (ABE). In some embodiments, splice disruption is achieved with a cytidine base editor (CBE). In some embodiments, base editors (e.g., ABE, CBE, or CABE) are used to edit exons by creating STOP codons.

In some embodiments, the modification generates a premature stop codon in the endogenous genes. In some embodiments, the STOP codon silences target protein expression. In some embodiments, the modification is a single base modification. In some embodiments, the modification is generated by base editing. The premature stop codon may be generated in an exon, an intron, or an untranslated region. In some embodiments, base editing may be used to introduce more than one STOP codon, in one or more alternative reading frames. In some embodiments, the stop codon is generated by a adenosine base editor (ABE). In some embodiments, the stop codon is generated by a cytidine base editor (CBE). In some embodiments, the CBE generates any one of the following edits (shown in underlined font) to generate a STOP codon: CAG→TAG; CAA→TAA; CGA→TGA; TGG→TGA; TGG→TAG; or TGG→TAA.

In some embodiments, modification/base edits may be introduced at a 3′-UTR, for example, in a poly adenylation (poly-A) site. In some embodiments, base editing may be performed on a 5′-UTR region.

### Delivery Systems

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., cytidine or adenine deaminase) can be delivered by vectors (e.g., viral or non-viral vectors), or by naked DNA, DNA complexes, lipid nanoparticles, or a combination of the aforementioned compositions. A base editor system may be delivered to a cell using any methods available in the art including, but not limited to, physical methods (e.g., electroporation, particle gun, calcium phosphate transfection), viral methods, non-viral methods (e.g., liposomes, cationic methods, lipid nanoparticles, polymeric nanoparticles), or biological non-viral methods (e.g., attenuated bacterial, engineered bacteriophages, mammalian virus-like particles, biological liposomes, erythrocyte ghosts, exosomes).

Nanoparticles, which can be organic or inorganic, are useful for delivering a base editor system or component thereof. Nanoparticles are well known in the art and any suitable nanoparticle can be used to deliver a base editor system or component thereof, or a nucleic acid molecule encoding such components. In one example, organic (e.g., lipid and/or polymer) nanoparticles are suitable for use as delivery vehicles in certain embodiments of this disclosure. Non-limiting examples of lipid nanoparticles suitable for use in the methods of the present disclosure include those described in International Patent Application Publications No. WO2022140239, WO2022140252, WO2022140238, WO2022159421, WO2022159472, WO2022159475, WO2022159463, WO2021113365, WO2024019936, and WO2021141969, the disclosures of each of which is incorporated herein by reference in its entirety for all purposes.

### Viral Vectors

A base editor described herein can be delivered with a viral vector. In some embodiments, a base editor disclosed herein can be encoded on a nucleic acid that is contained in a viral vector. In some embodiments, one or more components of the base editor system can be encoded on one or more viral vectors.

Viral vectors can include lentivirus (e.g., HIV and FIV-based vectors), Adenovirus (e.g., AD100), Retrovirus (e.g., Maloney murine leukemia virus, MML-V), herpesvirus vectors (e.g., HSV-2), rabies virus (see, e.g., U.S. Patent Application Publication No. US 2022/0290164 A1, the disclosure of which is incorporated herein by reference in its entirety for all purposes), and Adeno-associated viruses (AAVs), or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

Viral vectors can be selected based on the application. For example, for in vivo gene delivery, AAV can be advantageous over other viral vectors. In some embodiments, AAV allows low toxicity, which can be due to the purification method not requiring ultra-centrifugation of cell particles that can activate the immune response. In some embodiments, AAV allows low probability of causing insertional mutagenesis because it doesn't integrate into the host genome. Adenoviruses are commonly used as vaccines because of the strong immunogenic response they induce. Packaging capacity of the viral vectors can limit the size of the base editor that can be packaged into the vector.

AAV has a packaging capacity of about 4.5 Kb or 4.75 Kb including two 145 base inverted terminal repeats (ITRs). This means disclosed base editor as well as a promoter and transcription terminator can fit into a single viral vector. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed base editor which is shorter in length than conventional base editors. In some examples, the base editors are less than 4 kb. Disclosed base editors can be less than 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb. In some embodiments, the disclosed base editors are 4.5 kb or less in length.

An AAV can be AAV1, AAV2, AAV5, AAV6, AAV9, PHP.EB, PHP.B, AAV.CAP-B10, AAV, CAP-B22, AAV-rh10, a PAL family AAV, or any combination thereof. In embodiments, the AAV is capable of crossing the blood-brain barrier (see, e.g., those AAV vectors disclosed in Liu, et al. “Crossing the blood-brain barrier with AAV vectors,” Metabolic Brain Disease, 36:45-52 (2021), the disclosure of which is incorporated herein by reference in its entirety for all purposes). One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82:5887-5911 (2008)).

In some embodiments, the AAV vector contains a PAL family AAV capsid (see, Stanton, A., et al. Med 4:31-50 (2023) (doi: doi.org/10.1016/j.medj.2022.11.002), the disclosure of which is incorporated herein by reference in its entirety for all purposes). In some cases, the AAV PAL family AAV capsid contains the below AAV9 VP1 capsid amino acid sequence (UniProt Accession No. Q6JC40) with one of the 7-mers listed in Table 8 below inserted between amino acid positions Q588 and A589, which are shown in bold in the below sequence. In some embodiments, the AAV PAL family AAV capsid contains the below AAV9 VP1 capsid amino acid sequence with the amino acid alterations A587D and Q588G and one of the 7-mers listed in Table 8 inserted between amino acid positions G588 and A589.

>AAV9 VP1 capsid amino acid sequence: tr|Q6JC40|Q6JC40_9VIRU Capsid protein VP1
OS = Adeno-associated virus 9 OX = 235455 GN = cap PE = 1 SV = 1
(SEQ ID NO: 3559)
MAADGYLPDWLEDNLSEGIREWWALKPGAPQPKANQQHQDNARGLVLPGYKYLGPGNGLDKG
EPVNAADAAALEHDKAYDQQLKAGDNPYLKYNHADAEFQERLKEDTSFGGNLGRAVFQAKKR
LLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWA
LPTYNNHLYKQISNSTSGGSSNDNAYFGYSTPWGYFDFNRFHCHFSPRDWQRLINNNWGFRP
KRLNFKLFNIQVKEVTDNNGVKTIANNLTSTVQVFTDSDYQLPYVLGSAHEGCLPPFPADVF
MIPQYGYLTLNDGSQAVGRSSFYCLEYFPSQMLRTGNNFQFSYEFENVPFHSSYAHSQSLDR
LMNPLIDQYLYYLSKTINGSGQNQQTLKFSVAGPSNMAVQGRNYIPGPSYRQQRVSTTVTON
NNSEFAWPGASSWALNGRNSLMNPGPAMASHKEGEDRFFPLSGSLIFGKQGTGRDNVDADKV
MITNEEEIKTTNPVATESYGQVATNHQSAQAQAQTGWVQNQGILPGMVWQDRDVYLQGPIWA
KIPHTDGNFHPSPLMGGFGMKHPPPQILIKNTPVPADPPTAFNKDKLNSFITQYSTGQVSVE
IEWELQKENSKRWNPEIQYTSNYYKSNNVEFAVNTEGVYSEPRPIGTRYLTRNL

TABLE 8
PAL family AAV vector inserts.

| 7-mer | SEQ ID NO |
|---|---|
| RSVGSVY | 3531 |
| KTVGTVY | 3532 |
| RYLGDAS | 3533 |
| WVLPSGG | 3534 |
| VTVGSIY | 3535 |
| VRGSSIL | 3536 |
| REQQKLW | 3537 |
| ASNPGRW | 3538 |
| SLDKPFK | 3539 |
| TLAVPFK | 3540 |
| WTLESGH | 3541 |
| REQKKLW | 3542 |
| PTQGTVR | 3543 |
| PTQGTFR | 3544 |
| PSQGTLR | 3545 |
| NLGAALS | 3546 |
| PKPSHGE | 3547 |
| PTPGTLR | 3548 |
| PTQGTLR | 3549 |
| QDGPAVK | 3550 |
| PNQGTLR | 3551 |
| ESLAGVR | 3552 |
| TDALTTK | 3553 |
| TDAGDGK | 3554 |
| MTGISIV | 3555 |
| NGYTEGR | 3556 |
| SLVTSST | 3557 |
| PTQGTIR | 3558 |

In some embodiments, lentiviral vectors are used to transduce a cell of interest with a polynucleotide encoding a base editor or base editor system as provided herein. Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors are contemplated.

Any RNA of the systems, for example a guide RNA or a base editor-encoding mRNA, can be delivered in the form of RNA. Base editor-encoding mRNA can be generated using in vitro transcription. For example, nuclease mRNA can be synthesized using a PCR cassette containing the following elements: T7 promoter, optional kozak sequence (GCCACC), nuclease sequence, and 3′ UTR such as a 3′ UTR from beta globin-poly A tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

### Non-Viral Platforms for Gene Transfer

Non-viral platforms for introducing a heterologous polynucleotide into a cell of interest are known in the art.

For example, the disclosure provides a method of inserting a heterologous polynucleotide into the genome of a cell using a Cas9 or Cas12 (e.g., Cas12b) ribonucleoprotein complex (RNP)-DNA template complex where an RNP including a Cas9 or Cas12 nuclease domain and a guide RNA, wherein the guide RNA specifically hybridizes to a target region of the genome of the cell, and wherein the Cas nuclease domain cleaves the target region to create an insertion site in the genome of the cell. A DNA template is then used to introduce a heterologous polynucleotide. In embodiments, the DNA template is a double-stranded or single-stranded DNA template, wherein the size of the DNA template is about 200 nucleotides or is greater than about 200 nucleotides, wherein the 5′ and 3′ ends of the DNA template comprise nucleotide sequences that are homologous to genomic sequences flanking the insertion site. In some embodiments, the DNA template is a single-stranded circular DNA template. In embodiments, the molar ratio of RNP to DNA template in the complex is from about 3:1 to about 100:1.

In some embodiments, the DNA template is a linear DNA template. In some examples, the DNA template is a single-stranded DNA template. In certain embodiments, the single-stranded DNA template is a pure single-stranded DNA template. In some embodiments, the single stranded DNA template is a single-stranded oligodeoxynucleotide (ssODN).

In other embodiments, a single-stranded DNA (ssDNA) can produce efficient homology-directed repair (HDR) with minimal off-target integration. In one embodiment, an ssDNA phage is used to efficiently and inexpensively produce long circular ssDNA (cssDNA) donors. These cssDNA donors serve as efficient HDR templates when used with Cas9 or Cas12 (e.g., Cas12a, Cas12b), with integration frequencies superior to linear ssDNA (IssDNA) donors.

In some embodiments, a heterologous polynucleotide may be inserted into the genome of a cell using a transposable element such as a transposon, as described, for example, in Tipanee, et al. Human Gene Therapy, November 2017, 1087-1104, DOI: 10.1089/hum.2017.128. Transposable elements are divided into two categories: retrotransposons and DNA transposons. Transposable elements can alter the genome of the host cells through insertions, duplications, deletions, and translocations. Retrotransposons are described as mobile elements that employ an RNA intermediate that is first reverse transcribed into a complementary single-stranded (c) DNA strand by a reverse transcriptase encoded by the retrotransposon. Subsequently, the single-stranded DNA is converted into a double-stranded DNA that then integrates into the host genome. This so-called “replicative mechanism” yields several new copies of retrotransposons expanding throughout the target genome over evolutionary time. Retrotransposons are categorized into many subtypes according to the DNA sequences of the long terminal repeats and its open reading frames. Retrotransposons were employed to enable transgene integration into the target cell DNA, in some cases relying on adenoviral delivery. Alternatively, DNA transposons translocate via a “non-replicative mechanism,” whereby two Terminal Inverted Repeats (TIRs) are recognized and cleaved by a transposase enzyme, releasing the cognate DNA transposons with free DNA ends. The excised DNA transposons then integrate into a new genomic region where target sites are recognized and cut by the same transposase. This cut-and-paste mechanism usually duplicates DNA target sites upon insertion, leaving target site duplications (TSDs). Non-limiting examples of transposons include the Sleeping Beauty (SB) transposon, the piggyBac (PB) transposon, and Tol2 transposable elements.

### Inteins

Inteins (intervening protein) are auto-processing domains found in a variety of diverse organisms, which carry out a process known as protein splicing.

Non-limiting examples of inteins include any intein or intein-pair known in the art, which include a synthetic intein based on the dnaE intein, the Cfa-N (e.g., split intein-N) and Cfa-C (e.g., split intein-C) intein pair, has been described (e.g., in Stevens et al., J Am Chem Soc. 2016 Feb. 24; 138 (7): 2162-5, incorporated herein by reference), and DnaE. Non-limiting examples of pairs of inteins that may be used in accordance with the present disclosure include: Cfa DnaE intein, Ssp GyrB intein, Ssp DnaX intein, Ter DnaE3 intein, Ter Thy X intein, Rma DnaB intein and Cne Prp8 intein (e.g., as described in U.S. Pat. No. 8,394,604, incorporated herein by reference). Exemplary nucleotide and amino acid sequences of inteins are provided in the Sequence Listing at SEQ ID NOs: 370-377 and 389-424. Inteins suitable for use in embodiments of the present disclosure and methods for use thereof are described in U.S. Pat. No. 10,526,401, International Patent Application Publication No. WO 2013/045632, WO 2024/073385, or WO 2020/051561, and in U.S. Patent Application Publication No. US 2020/0055900, the full disclosures of which are incorporated herein by reference in their entireties by reference for all purposes.

Intein-N and intein-C may be fused to the N-terminal portion of a split Cas9 and the C-terminal portion of the split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N-[N-terminal portion of the split Cas9]-[intein-N]-C. In some embodiments, an intein-C is fused to the N-terminus of the C-terminal portion of the split Cas9, i.e., to form a structure of N-[intein-C]-[C-terminal portion of the split Cas9]-C. In embodiments, a base editor is encoded by two polynucleotides, where one polynucleotide encodes a fragment of the base editor fused to an intein-N and another polynucleotide encodes a fragment of the base editor fused to an intein-C. Methods for designing and using inteins are known in the art and described, for example by WO2014004336, WO2017132580, WO2013045632A1, US20150344549, and US20180127780, each of which is incorporated herein by reference in their entirety.

In some embodiments, an ABE was split into N- and C-terminal fragments at Ala, Ser, Thr, or Cys residues within selected regions of SpCas9. These regions correspond to loop regions identified by Cas9 crystal structure analysis.

The N-terminal fragment is fused at the C-terminus to an intein-N and the C-terminal fragment is fused to an intein-C at an N-terminal amino acid selected from the group consisting of S303, T310, T313, S355, A456, S460, A463, T466, S469, T472, T474, C574, S577, A589, and S590, referenced to SEQ ID NO: 197. In various embodiments, the SpCas9 is split between amino acid positions 302 and 303, 309 and 310, 312 and 313, 354 and 355, 455 and 456, 459 and 460, 462 and 463, 465 and 466, 468 and 469, 471 and 472, 473 and 474, 573 and 574, 576 and 577, 588 and 589, or 589 and 590, referenced to SEQ ID NO: 197 to yield an N-terminal fragment and a C-terminal fragment, where the N-terminal fragment is fused at the C-terminus to a an intein-N and where the C-terminal fragment is fused at the N-terminus to an intein-C.

### Pharmaceutical Compositions

In some aspects, the present disclosure provides a pharmaceutical composition comprising any of the cells, polynucleotides, vectors, base editors, base editor systems, guide polynucleotides, fusion proteins, complexes, or the fusion protein-guide polynucleotide complexes described herein.

The pharmaceutical compositions of the present disclosure can be prepared in accordance with known techniques. See, e.g., Remington, The Science And Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

In some embodiments, the pharmaceutical composition is formulated for delivery to a subject. Suitable routes of administrating the pharmaceutical composition described herein include, without limitation: intravitreal, subretinal, suprachoroidal, topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site or source of a disease (e.g., an organ, such as a liver). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, any of the fusion proteins, gRNAs, and/or complexes described herein are provided as part of a pharmaceutical composition. In some embodiments, the pharmaceutical composition comprises any of the fusion proteins or complexes provided herein. In some embodiments pharmaceutical composition comprises a gRNA, a nucleic acid programmable DNA binding protein, a cationic lipid, and a pharmaceutically acceptable excipient. In embodiments, pharmaceutical compositions comprise a lipid nanoparticle and a pharmaceutically acceptable excipient. In embodiments, the lipid nanoparticle contains a gRNA, a base editor, a complex, a base editor system, or a component thereof of the present disclosure, and/or one or more polynucleotides encoding the same. Pharmaceutical compositions can optionally comprise one or more additional therapeutically active substances.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Methods of Treatment

Some aspects of the present disclosure provide methods of treating a subject in need, the method comprising administering to a subject in need an effective therapeutic amount of a pharmaceutical composition as described herein. More specifically, the methods of treatment include administering to a subject in need thereof one or more pharmaceutical compositions comprising one or more cells having at least one edited gene. In other embodiments, the methods of the disclosure comprise expressing or introducing into a cell a base editor polypeptide and one or more guide RNAs capable of targeting a nucleic acid molecule encoding at least one polypeptide.

One of ordinary skill in the art would recognize that multiple administrations of the pharmaceutical compositions contemplated in particular embodiments may be required to affect the desired therapy. For example, a composition may be administered to the subject 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

### Combination Therapy

In various embodiments, methods of the present disclosure involve administering an inhibitor of a component of the complement system (e.g., of component C3 or factor B). In embodiments, a pharmaceutical composition of the disclosure contains an inhibitor of complement component C3. In embodiments, a pharmaceutical composition of the disclosure contains an inhibitor of factor B. In some embodiments, the complement inhibitor is compstatin or a compstatin analog or mimetic.

Drugs that inhibit complement system function are available and may be used in methods of the present disclosure. In embodiments, the drug is a small molecule, a nucleic acid molecule, a peptide, or an antibody. Non-limiting examples of such drugs include, but are not limited to, EMPAVELI (Pegcetacoplan; APL-2), which targets C3, Danicopan (ACH-4471), which targets Factor D, Iptacopan (LPN 023), which targets factor B, siRNAs (Arrowhead ARO-C3), which targets C3, Amy 101, which targets C3, Eculizumab, which targets C5, Ravulizumab, which targets C5, Cemdisiran, which targets C5, OMS-721, which targets MASP2, IFX-1, which targets C5a, and Avacopan, which targets C5aR1.

Compstatin is a cyclic peptide that binds to C3 and inhibits complement activation. U.S. Pat. No. 6,319,897 describes a peptide having the sequence I [CVVQDWGHHRC]T (SEQ ID NO: 3105), with the disulfide bond between the two cysteines denoted by brackets. Morikis, et al., Protein Sci., 7 (3): 619-27, 1998) also describe a compstatin. In some instances, compstatin is amidated at the C-terminus. Compstatin analogs, mimetics, derivatives thereof, and/or compositions containing the same suitable for use in the methods and compositions of the present disclosure include those described in WO2021007111 (PCT/US2020/040741); WO2021011927 (PCT/US2020/042676); WO2004026328 (PCT/US2003/029653); Morikis, D., et al., Biochem Soc Trans. 32 (Pt 1): 28-32, 2004, Mallik, B., et al., J. Med. Chem., 274-286, 2005; Katragadda, M., et al. J. Med. Chem., 49:4616-4622, 2006; WO2007062249 (PCT/US2006/045539); WO2007044668 (PCT/US2006/039397); WO2009046198 (PCT/US2008/078593); WO2010127336 (PCT/US2010/033345); WO2012155107; WO 2014078731; WO2019166411; WO2009121065; WO2021163654; WO2021142171; WO2017062879; WO2014152391; WO2014028861; WO2018187813; WO2019089653; WO2016049385; WO2018075373; WO2019118938; WO2022061304; WO2012006599; WO2011163394; WO2012178083; U.S. Pat. Nos. 9,291,622; 10,407,466; 8,580,735; and Hillmen, et al. “Pegcetacoplan versus Eculizumab in Paroxysmal Nocturnal Hemoglobinuria,” N Engl J Med. 2021 Mar. 18; 384 (11): 1028-1037; the disclosures of all of which are incorporated herein by reference in their entireties for all purposes. In certain embodiments, a compstatin analog is pegcetacoplan (“APL-2”). Pegcetacoplan is also referred to as Poly (oxy-1,2-ethanediyl), α-hydro-ω-hydroxy-, 15,15′-diester with N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy) ethoxy]acetyl-N⁶-carboxy-L-lysinamide cyclic (2->12)-(disulfide); or O,O′-bis [(S²,S¹²-cyclo {N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy) ethoxy]acetyl-L-lysinamide})-N^{6.15}-carbonyl]polyethylene glycol (n=800-1100).

In some embodiments, a complement inhibitor is an antibody, e.g., an anti-C3 antibody, or a fragment thereof. In some embodiments, an antibody fragment may be used to inhibit C3 activation. The antibody fragment may be Fab′, Fab′ (2), Fv, or a single chain Fv. In some embodiments, the anti-C3 antibody is monoclonal. In some embodiments, the anti-antibody is polyclonal. In some embodiments, the anti-C3 antibody is de-immunized. In some embodiments the anti-C3 antibody is a fully human monoclonal antibody.

In some instances, a complement inhibitor is an inhibitory polynucleotide (e.g., an siRNA), such as those described in WO2021163654, the disclosure of which is incorporated herein in its entirety for all purposes.

In some embodiments, a complement inhibitor is a polypeptide inhibitor and/or a nucleic acid aptamer (see, e.g., U.S. Publ. No. 20030191084). Exemplary polypeptide inhibitors include an enzyme that degrades C3 or C3b (see, e.g., U.S. Pat. No. 6,676,943).

### Kits

The invention provides kits for use in treating a subject to reduce complement activation. In some embodiments, the kit further includes a base editor system or a polynucleotide encoding a base editor system, wherein the base editor polypeptide system a nucleic acid programmable DNA binding protein (napDNAbp), a deaminase, and a guide RNA. In some embodiments, the napDNAbp is Cas9 or Cas12. In some embodiments, the polynucleotide encoding the base editor is a mRNA sequence. In some embodiments, the deaminase is a cytidine deaminase or an adenosine deaminase. In some embodiments, the kit comprises a guide RNA and/or base editor system and instructions regarding the use of the guide RNA and/or base editor system.

The kits may further comprise written instructions for using a base editor, base editor system and/or edited cell as described herein. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit comprises instructions in the form of a label or separate insert (package insert) for suitable operational parameters. In yet another embodiment, the kit comprises one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of embodiments of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing embodiments of the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: Base Editing of a Complement Factor B (CFB) Polynucleotide to Disrupt a Splice Site

Experiments were undertaken to develop base editor systems for altering a splice site of a CFB polynucleotide to reduce levels of functional CFB polypeptides in a cell or subject. The base editor systems were evaluated both in vitro and in vivo. It can be advantageous to silence or knock-out CFB in a subject as part of a treatment for a disease or disorder associated with inappropriate activation of the complement system.

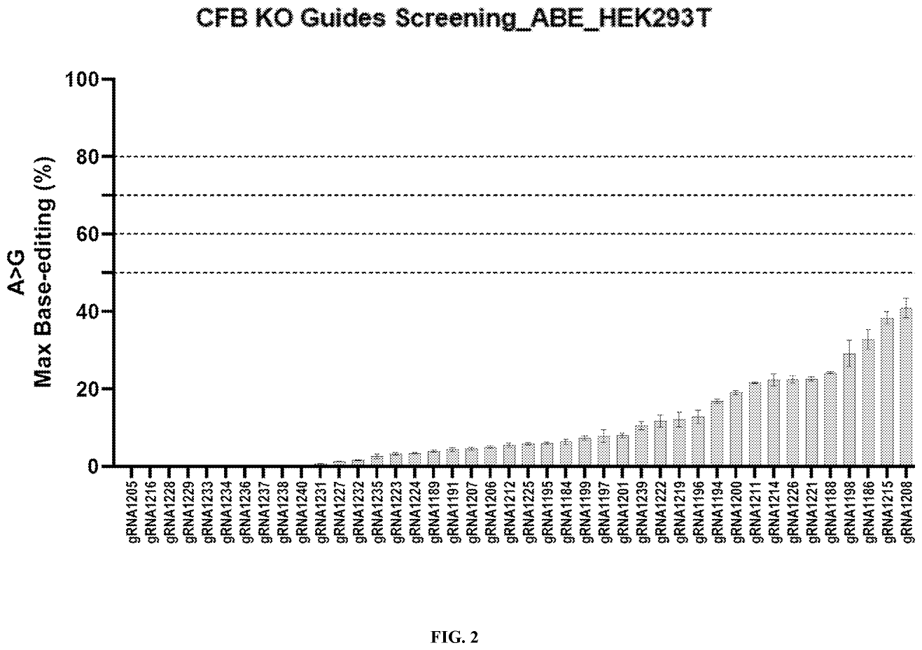

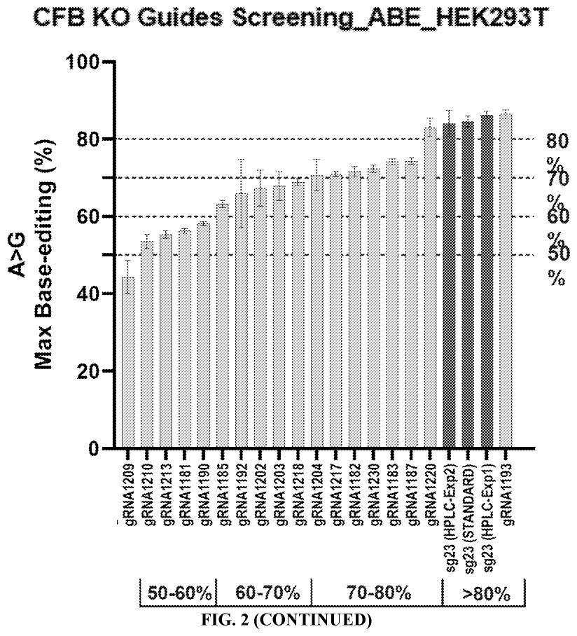

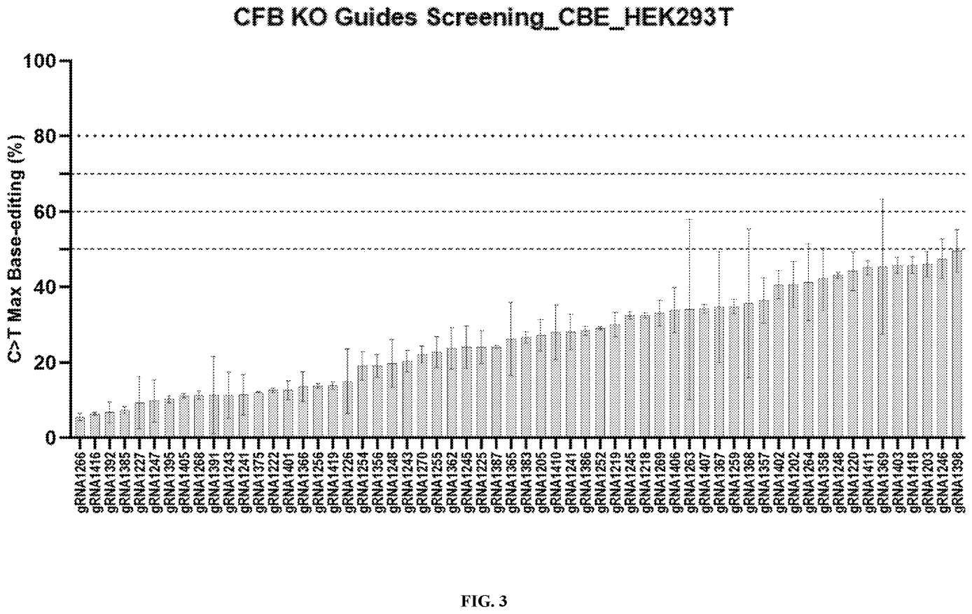

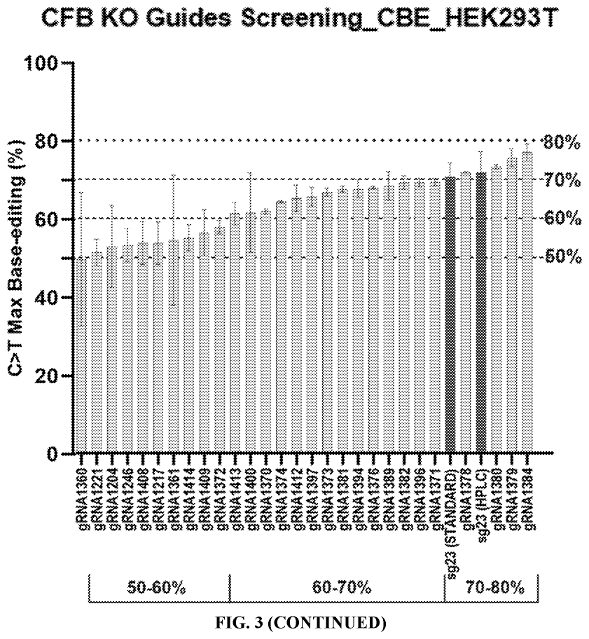

Base editor systems were developed for disrupting a splice site of a CFB polynucleotide or introduce a stop codon and evaluated using HEK293T cells. The base editor systems contained a cytidine deaminase base editor (CBE) or an adenosine deaminase base editor (ABE) (e.g., ABE_NGC_20 nt_3-9_008, ABE_NGG_20 nt_3-9_002, ABE_NGA_20 nt_3-9_005, ABE_NNGRRT_21 nt_5-14_011, ABE_NNNRRT 21 nt 5-14_014, CBE_NNGRRT_21 nt_3-12_012, CBE_NNNRRT_21 nt_3-12_015, CBE_NGG_20 nt_4-9_003, or CBE_NGA_20 nt_4-9_006, where, throughout the Examples, the terms “NGC,” “NGG,” “NGA,” “NNGRRT,” and “NNNRRT” indicate the PAM specificity of the napDNAbp of each base editor, “ABE” indicates an adenosine deaminase base editor, “CBE” indicates a cytidine deaminase base editor, and “20 nt” or “21 nt” indicates the length of the spacer of the guide polynucleotide used in combination with the base editor) and a guide polynucleotides targeting a splice site of a CFB polynucleotide for base editing. Some of the base editor systems containing a CBE included a guide polynucleotide targeting the base editor to introduce a nucleotide modification to the CFB polynucleotide that resulted in the introduction of a new stop codon. The HEK293T cells were administered 800 ng total of the guide polynucleotide (200 ng) and mRNA encoding the base editor (600 ng). Seventeen (17) of the base editor systems containing an adenosine deaminase base editor and a guide polynucleotide were associated with maximum percents A to G base editing of greater than 50% (FIG. 2; Table 8). Twenty-seven (27) of the base editor systems containing a cytidine deaminase base editor and a guide polynucleotide were associated with maximum percents C to T base editing of greater than 50% (FIG. 3). The following guide polynucleotides were associated with maximum percents A to G base editing of greater than 50%: gRNA1210, gRNA1213, gRNA1181, gRNA1190, gRNA1185, gRNA1192, gRNA1202, gRNA1203, gRNA1218, gRNA1204, gRNA1217, gRNA1182, gRNA1230, gRNA1183, gRNA1187, gRNA1220, gRNA1193, gRNA1221, gRNA1204, gRNA1246, gRNA1408, gRNA1217, gRNA1361, gRNA1414, gRNA1409, gRNA1372, gRNA1413, gRNA1400, gRNA1370, gRNA1374, gRNA1412, gRNA1397, gRNA1373, gRNA1381, gRNA1394, gRNA1376, gRNA1389, gRNA1382, gRNA1396, gRNA1371, gRNA1378, gRNA1380, gRNA1379, and gRNA1384.

TABLE 8
Base editing rates associated with guides associated with
maximum percents A to G base editing of greater than 50%.¹⁰

| | | | | Cyno X | |
|---|---|---|---|---|---|
| | | | Target | Reac- | |
| Guides* | Average | SD | Site | tivity | Location |
| gRNA1193 | 86.43 | 1.1 | TSBTx3826 | Y | Exon 10-5′ |
| sg23 (HPLC Exp1) | 86.27 | 0.92 | | | |
| sg23 (STANDARD) | 84.55 | 1.43 | | | |
| sg23 (HPLC) | 84.03 | 3.39 | | | |
| gRNA1220 | 83.01 | 2.41 | TSBTx3853 | N | Exon 10-3′ |
| gRNA1187 | 74.4 | 0.87 | TSBTx3820 | N | Exon 1-3′ |
| gRNA1183 | 74.18 | 0.68 | TSBTx3816 | N | Exon 18-5′ |
| gRNA1230 | 72.38 | 0.93 | TSBTx3863 | Y | Exon 8-5′ |
| gRNA1182 | 71.61 | 1.36 | TSBTx3815 | Y | Exon 14-5′ |
| gRNA1217 | 71.06 | 0.67 | TSBTx3850 | Y | Exon 15-3′ |
| gRNA1204 | 70.7 | 4.01 | TSBTx3837 | N | Exon 11-3′ |
| gRNA1218 | 68.93 | 0.85 | TSBTx3851 | N | Exon 12-3′ |
| gRNA1203 | 67.87 | 3.65 | TSBTx3836 | N | Exon 14-3′ |
| gRNA1202 | 67.37 | 4.61 | TSBTx3835 | Y | Exon 16-3′ |
| gRNA1192 | 65.96 | 8.8 | TSBTx3825 | Y | Exon 10-5′ |
| gRNA1185 | 63.26 | 0.9 | TSBTx3818 | N | Exon 11-3′ |
| gRNA1190 | 58.23 | 0.51 | TSBTx3823 | Y | Exon 9-5′ |
| gRNA1181 | 56.32 | 0.7 | TSBTx3814 | Y | Exon 9-5′ |
| gRNA1213 | 55.35 | 0.94 | TSBTx3846 | Y | Exon 11-5′ |
| gRNA1210 | 53.52 | 1.84 | TSBTx3843 | Y | Exon 5-5′ |

¹⁰The term “Cyno X Reactivity” refers to whether a guide targets both a human CFB and a cyno CFB for base editing (i.e., identified as “Y” and is cross-reactive) or whether a guide only targets a human
*The terms “HPLC” and “STANDARD” refer to the methods by which the guides were purified. The sg23 guides were synthesized by Agilent and subsequently purified by Agilent using “HPLC” or “STANDARD” purification methods.

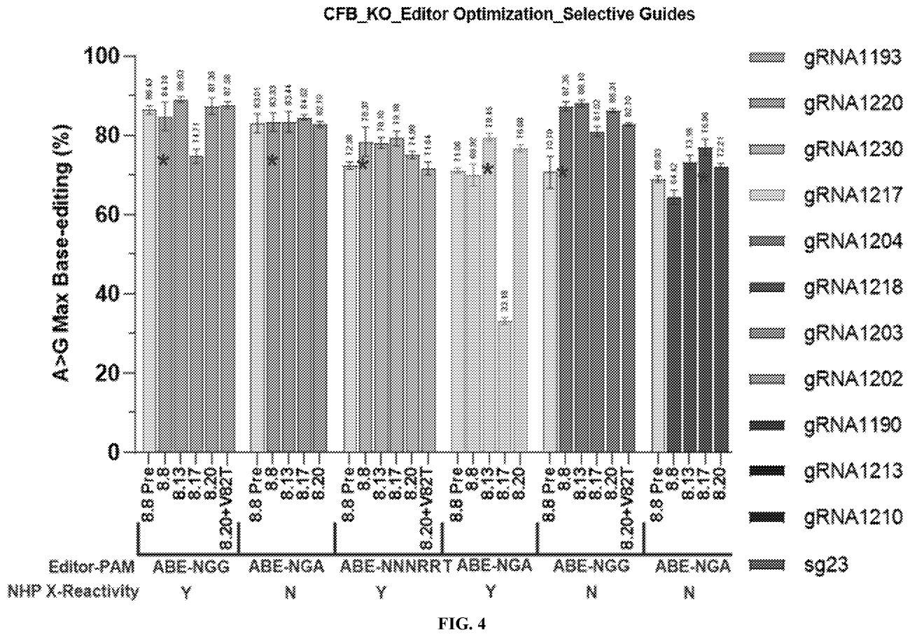

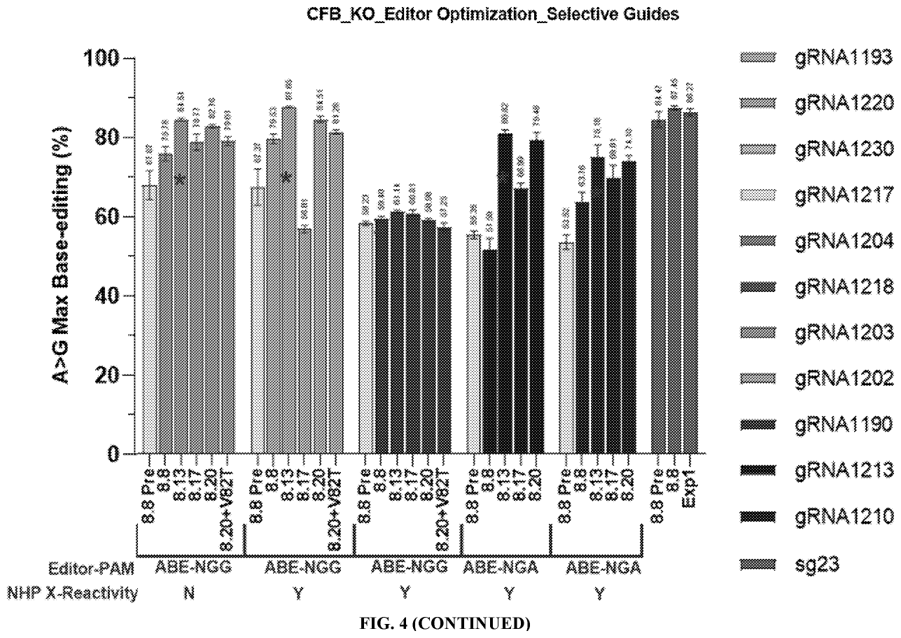

Having identified base editor systems associated with high base editing rates and containing one of the guides listed in Table 8, experiments were next undertaken to optimize the adenosine deaminase base editor used in the base editor systems. Maximum percent A to G base editing of a CFB polynucleotide was measured in HEK293T cells transfected with a guide polynucleotide and one of the adenosine deaminase base editors ABE8.8 Pre, ABE8.8, ABE8.13, ABE8.17, ABE8.20, and ABE8.20 with the adenosine deaminase amino acid 10 alteration V82T (ABE8.20+V82T) (see FIG. 4). The adenosine deaminase base editors each contained a Cas9 nickase domain that was capable of binding an NGG, NGA, or NNNRRT PAM sequence (see FIG. 4). A few of the base editor systems showed improved base editing rates when the adenosine deaminase base editor was changed to ABE8.13 or ABE8.17. The

CFB for base editing (i.e., identified as “N” and is not cross reactive). For each location listed in Table 8, the term “5” or “3” indicates the end of the exon closest to the splice site targeted for base editing. The term “SD” means standard deviation and the term “Average” indicates average maximum base editing measured in HEK293T cells.
following base editor systems showed good base editing rates, where, for each base editor system, the first term represents the guide polynucleotide, the letters following “ABE-” indicate the PAM specificity of the adenosine deaminase base editor, and the last numbers indicate the identity of the adenosine deaminase domain of the base editor, where 8.8 refers to TadA*8.8, 8.13 refers to TadA*8.13, and 8.17 refers to TadA*8.17: gRNA1193: ABE-NGG_8.8; gRNA1220: ABE-NGA_8.8; gRNA1230: ABE-NNNRRT_8.8; gRNA1217: ABE-NGA_8.13; gRNA1204: ABE-NGG_8.8; gRNA1218: ABE-NGA_8.17; gRNA1203: ABE-NGG_8.13; gRNA1202: ABE-NGG_8.13; gRNA1190: ABE-NGG_8.8; gRNA1213: ABE-NGA_8.13; and gRNA1210: ABE-NGA 8.13.

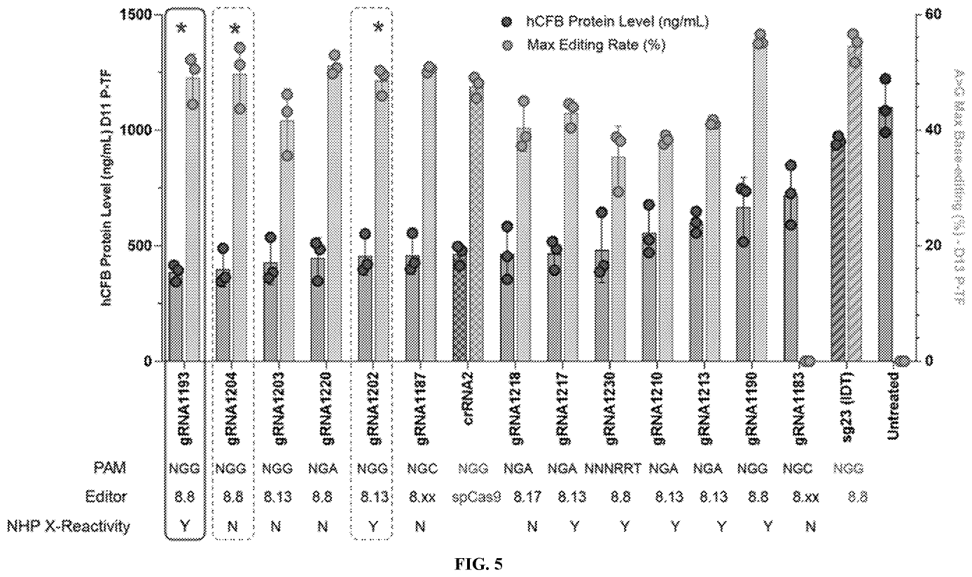

Optimized base editor systems showing good maximum percent A to G base editing of a CFB polynucleotide in HEK293T cells were further evaluated in primary human hepatocyte (PHH) co-cultures. PHH transfected with polynucleotides encoding the base editor systems (i.e., a guide polynucleotide and mRNA encoding the base editor) showed high maximum A to G base editing at day 13 post-transfection and reduced levels of human CFB (hCFB) at day 11 post-transfection (FIG. 5).

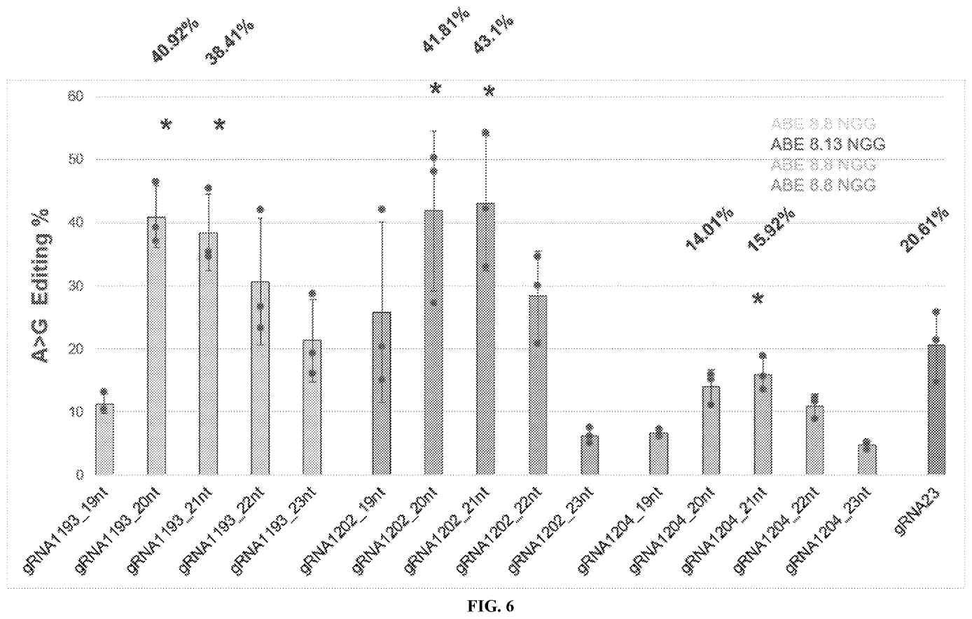

To further optimize the base editor systems, an experiment was undertaken to optimize the length of the spacers of the guide polynucleotides (FIG. 6) used in three of the base editor systems. Percent CFB polynucleotide A to G base editing was measured in HEK293T cells transfected with mRNA encoding an adenosine deaminase base editor with specificity for an NGG PAM and with a gRNA1193 (TSBTx3826), gRNA1202 (TSBTx3835), or gRNA1204 (TSBTx3837) guide polynucleotide containing a spacer with a length of 19 nt, 20 nt, 21 nt, 22 nt, or 23 nt (FIG. 6). The cells were transfected using 100 ng of the guide polynucleotide and 300 ng mRNA encoding the adenosine deaminase base editor. The highest percents CFB polynucleotide A to G base editing were associated with spacers that were 20 nt or 21 nt in length (FIG. 6).

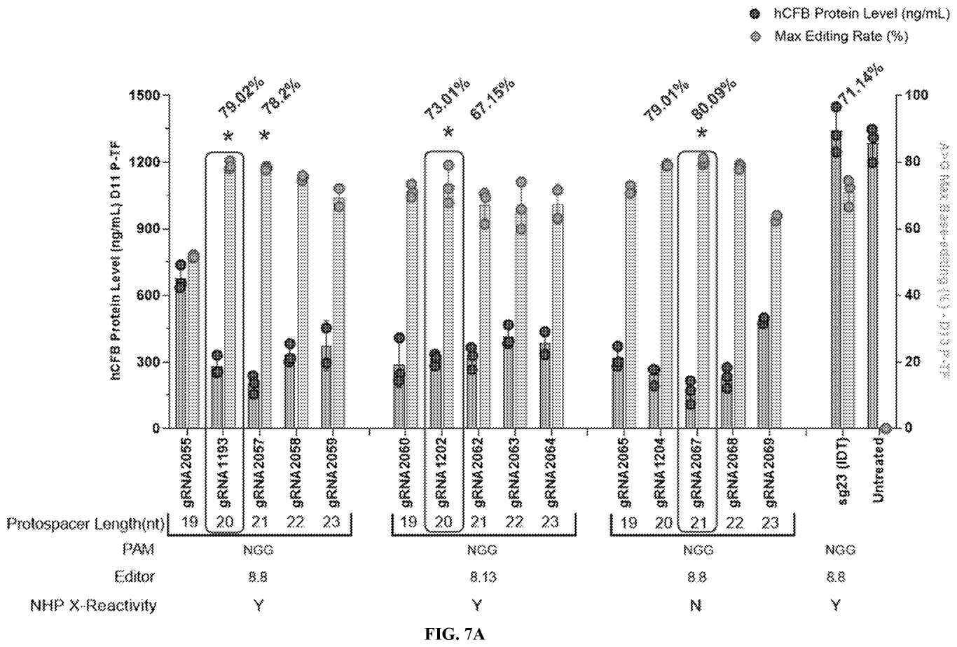

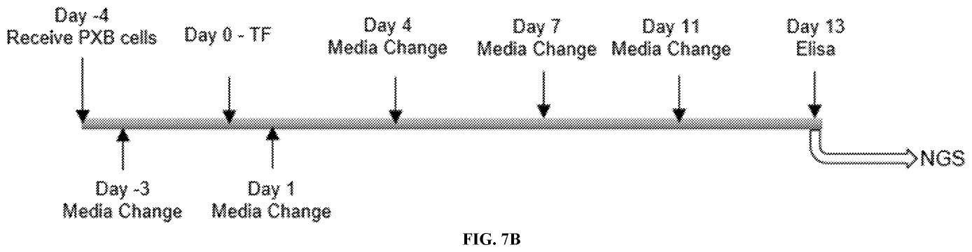

Optimal spacer length was further evaluated using human hepatocytes isolated from a PXB-mouse (PXB cells) (FIG. 7B). Percent CFB polynucleotide A to G base editing and human CFB (hCFB) protein levels were measured in PXB cells transfected with mRNA encoding an adenosine deaminase base editor (ABE8.8 or ABE8.13) with specificity for an NGG PAM and with a TSBTx3826 (e.g., gRNA1193), TSBTx3835 (e.g., gRNA1202), or TSBTx3837 (e.g., gRNA2067) guide polynucleotide containing a spacer with a length of 19 nt, 20 nt, 21 nt, 22 nt, or 23 nt (FIG. 7A). The cells were transfected using 150 ng of the guide polynucleotide and 450 ng mRNA encoding the adenosine deaminase base editor. The base editor systems were associated with high maximum percents A to G base editing and reductions in hCFB protein levels.

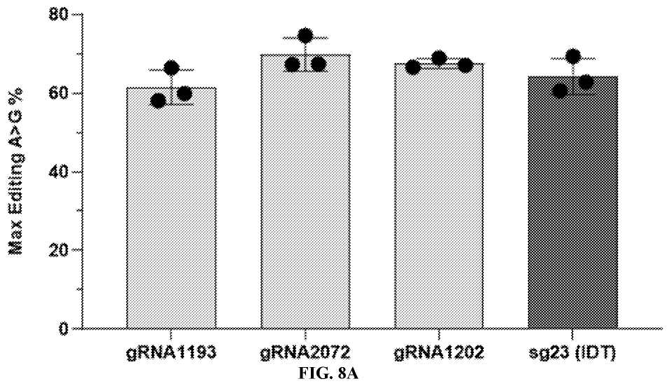

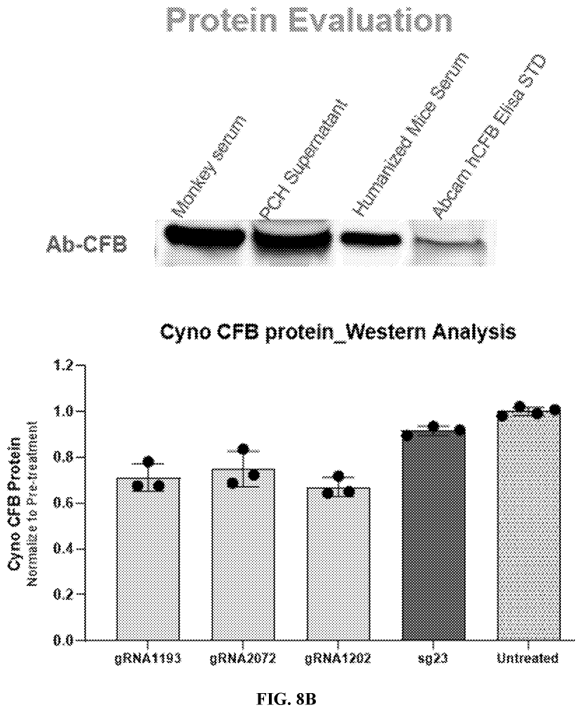

Editing efficiency for base editor systems containing guide polynucleotides cross-reactive (i.e., capable of targeting both a non-human primate and a human CFB polynucleotide for base editing) and cyno-surrogate (i.e., capable of targeting a non-human primate CFB polynucleotide for base editing) guide polynucleotides was evaluated using primary cyno hepatocyte (PCH) co-cultures (FIGS. 8A and 8B). The CFB co-cultures were transfected with mRNA encoding an adenosine deaminase base editor (ABE8.8 or ABE8.13) and the guide polynucleotide gRNA1193, gRNA2072, or gRNA1202. The base editor systems were associated with maximum percents CFB A to G editing of greater than about 60% (FIG. 8A) and with reductions in cyno CFB protein levels in the PCH (FIG. 8B).

Table 9 provides a description of the ABE-encoding mRNA molecules used in the examples and the ABEs encoded by the same.

TABLE 9
Description of mRNA molecules encoding base editors used in the Examples.
Throughout the disclosure, the term “MRNA” in each mRNA name may be replaced
with the term “m.”

| mRNA | | | |
|---|---|---|---|
| name | TadA* | Linker* | Cas9 |
| MRNA3171 | 8.8 | 32 aa (original) | WT |
| MRNA3534 | 8.8 | 32 aa (original) | WT |
| MRNA3535 | 8.13 | 32 aa (original) | WT |
| MRNA3536 | 8.17 | 32 aa (original) | WT |
| MRNA3537 | 8.20 | 32 aa (original) | WT |
| MRNA3538 | 8.8 + V82T | 32 aa (original) | WT |
| MRNA3539 | 8.8 + V82T + Y147T + | 32 aa (original) | WT |
| | Q154S | | |
| MRNA3540 | 8.17 + V82T | 32 aa (original) | WT |
| MRNA3541 | 8.17 + V82T + Y147T + | 32 aa (original) | WT |
| | Q154S | | |
| MRNA3542 | 8.20 + V82T | 32 aa (original) | WT |
| MRNA3543 | 8.20 + V82T + Y147T + | 32 aa (original) | WT |
| | Q154S | | |
| MRNA3544 | 8.13-m | 32 aa (original) | A1283D |
| MRNA3545 | 8.13-m | 32 aa (original) | A1283D + E1250K |
| MRNA3546 | 8.13-m | 32 aa (original) | Q844R, V842L, |
| | | | F846Y, L847M and |
| | | | 1852F (TurboCas9) |
| MRNA3547 | 8.13-m | KGPKPKKEESEK | A1283D |
| | | (SEQ ID NO: | |
| | | 3574) | |
| MRNA3548 | 8.13-m | KGPKPKKEESEK | A1283D + E1250K |
| | | (SEQ ID NO: | |
| | | 3574) | |
| MRNA3549 | 8.13-m | KGPKPKKEESEK | Q844R, V842L, |
| | | (SEQ ID NO: | F846Y, L847M and |
| | | 3574) | 1852F (TurboCas9) |

*The term “32 aa (original)” refers to a linker with the following amino acid sequence: SGGSSGGSSGSETPGTSESATPESSGGSSGGS (SEQ ID NO: 357).

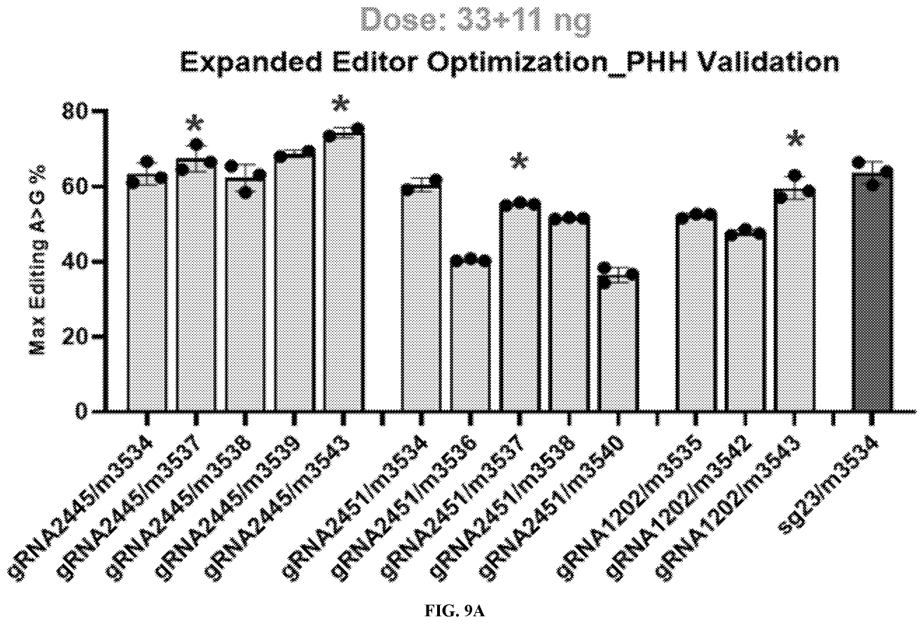

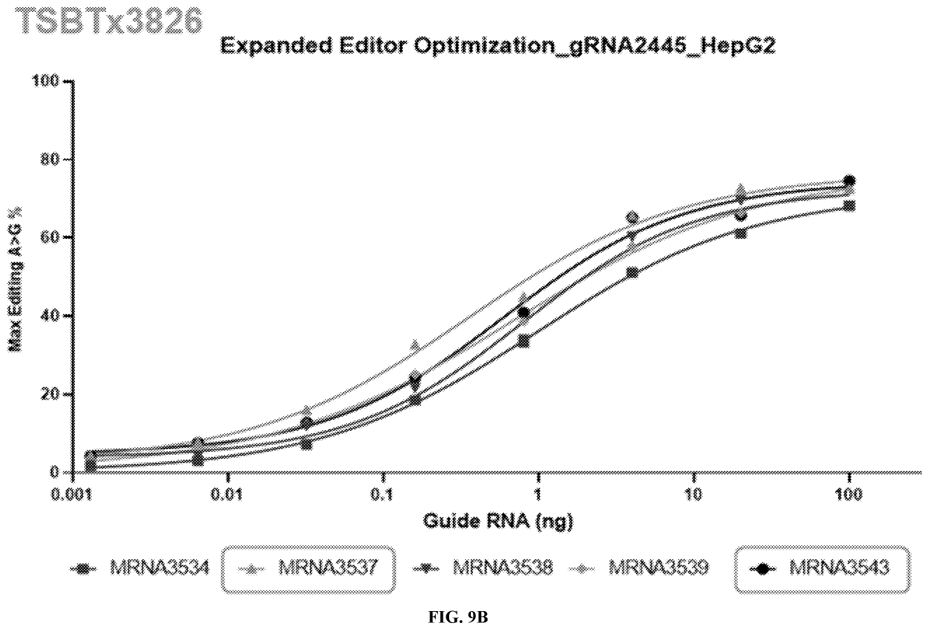

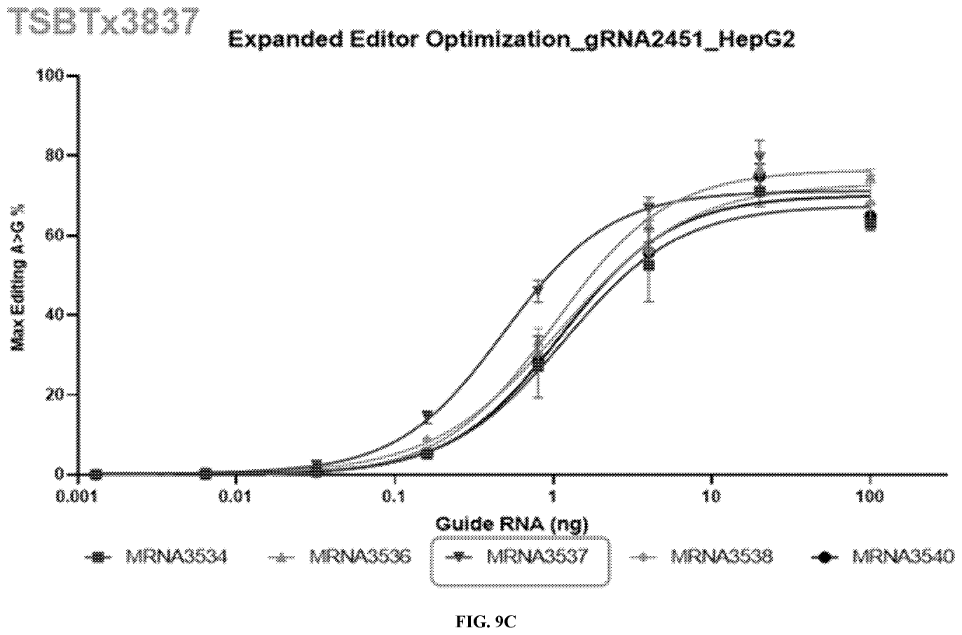

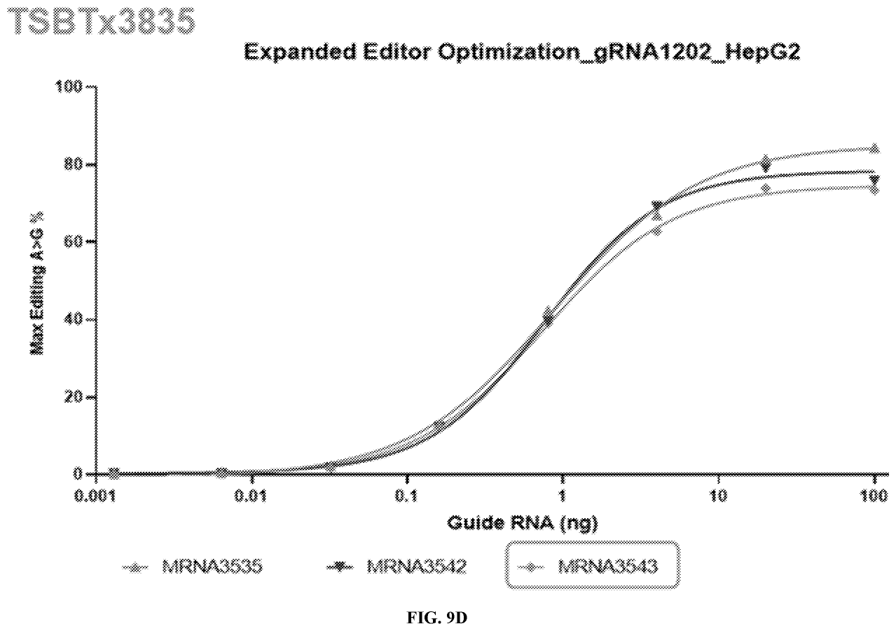

The base editor systems were further optimized by evaluating maximum percent CFB A to G base editing in HepG2 cells (FIGS. 9B-9D), which are human liver cancer cell line derived from the liver tissue of a 15-year-old Caucasian male with a hepatocellular carcinoma, and in primary human hepatocytes (FIG. 9A) transfected with different doses of a gRNA2445 (TSBTx3826; NLS nucleotide modification scheme), gRNA2451 (TSBTx3837; HM01 nucleotide modification scheme), or gRNA1202 (TSBTx3835; End-Mod nucleotide modification scheme) guide polynucleotide and a constant dose of one of the mRNA's of Table 9 encoding an adenosine deaminase base editor (FIGS. 9A-9D). The following base editor systems showed high maximum percents CFB A to G base editing, where the term following the underscores (“_”) and before the plus signs (“+”) indicate the guide polynucleotide nucleotide modification scheme: gRNA2445 (TSBTx3826)_NLS+ABE8.8; gRNA2445 (TSBTx3826)_NLS+ABE8.20; gRNA2445 (TSBTx3826)_NLS+ an ABE8.20 with a TadA*8.20 adenosine deaminase domain containing the alterations V82T, Y147T, and Q154S; gRNA2451 (TSBTx3837)_HM01+ABE8.8; gRNA2451 (TSBTx3837)_HM01+ABE8.20; gRNA1202 (TSBTx3835)_End-Mod+ABE8.13; gRNA1202 (TSBTx3835)_End-Mod+ an ABE8.20 with a TadA*8.20 adenosine deaminase domain containing the alterations V82T, Y147T, and Q154S.

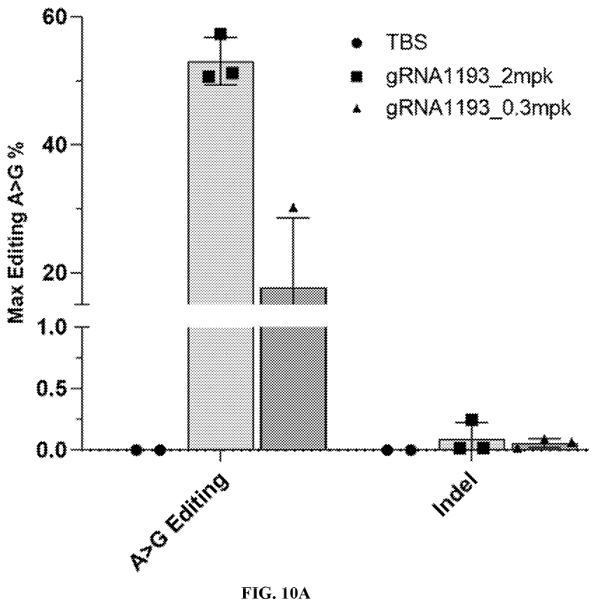

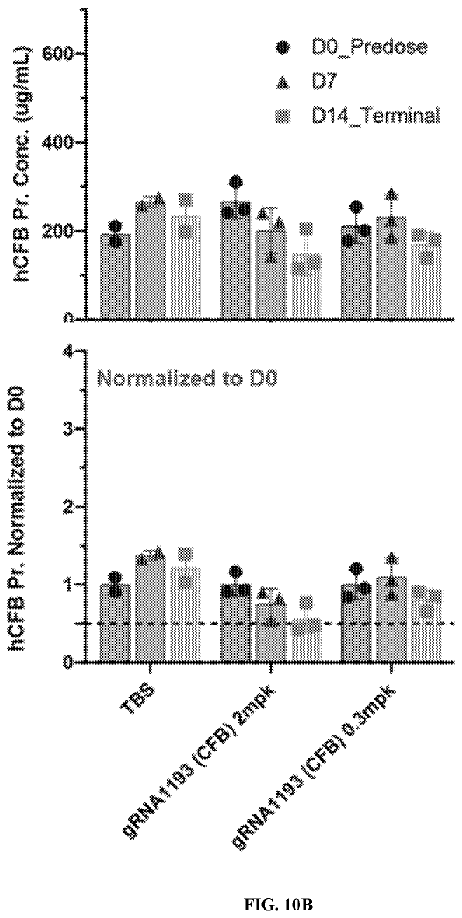

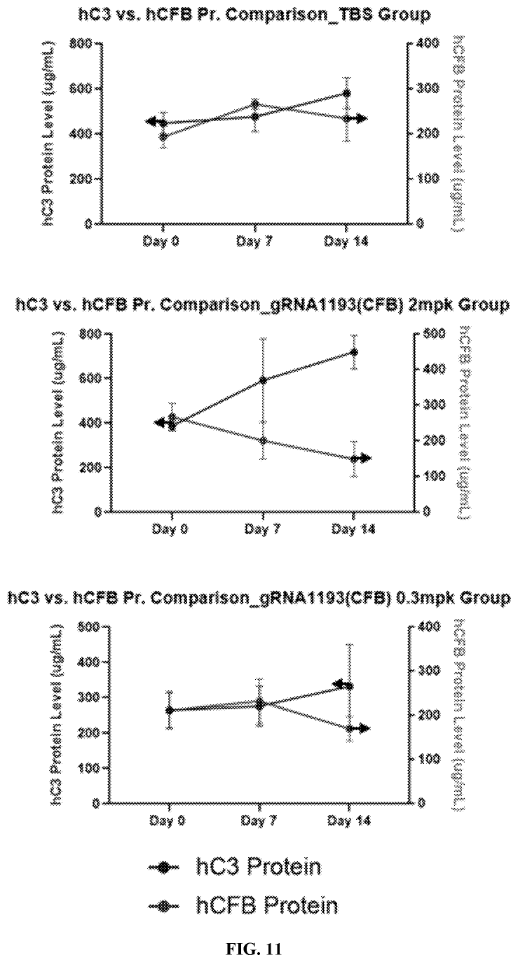

Experiments were next undertaken to evaluate the ability of the optimized base editor systems to mediate base editing of a complement factor B (CFB) polynucleotide in FRG™ liver-humanized mice. The FRG™ humanized mice were administered lipid nanoparticles containing mRNA encoding the adenosine deaminase base editor ABE8.8 and either 2 mg/kg (mpk) or 0.3 mpk of a gRNA1193 (TSBTx3826; end-mod nucleotide modification scheme) guide polynucleotide. Maximum percents A to G editing and percent indel formation (FIG. 10A) and human CFB (hCFB) levels (FIG. 10B) were measured in the mice prior to administration (i.e., day 0 pre-dose), at day 7 post-administration, and at day 14 post-administration (experiment termination). For a dose of 2.0 mpk of gRNA1193, a percent maximum A to G editing of about 53% was measured together with a reduction in CFB protein levels of about 45%. For a dose of 0.3 mpk of gRNA1193, a percent maximum A to G editing of about 18% was measured together with a reduction in CFB protein levels of about 20%. The base editor systems were associated with minimal indel formation. A time-dependent decrease in hCFB polypeptide levels was observed (FIG. 10B). Interestingly, it was observed that as factor B protein levels decreased in the mice, C3 protein levels increased but C3 mRNA levels remained unchanged at day 14 in the FRG™ humanized mice (FIG. 11). Without intending to be bound by theory, removing factor B may result in lower C3 convertase and, thus, slowed breakdown/consumption of C3 and an apparent increase in amount of protein level without impacting mRNA levels. Also, as C3 protein levels decreased, factor B mRNA and protein levels increased at day 14 in the FRG™ humanized mice (FIG. 11).

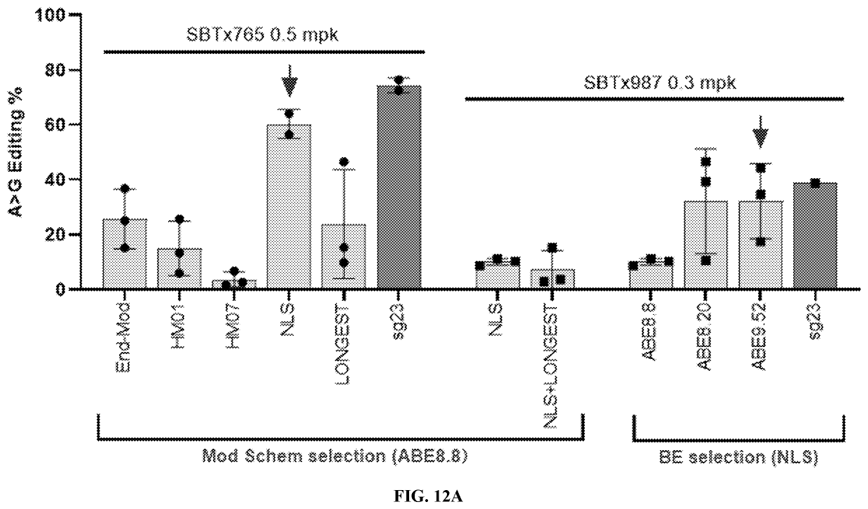

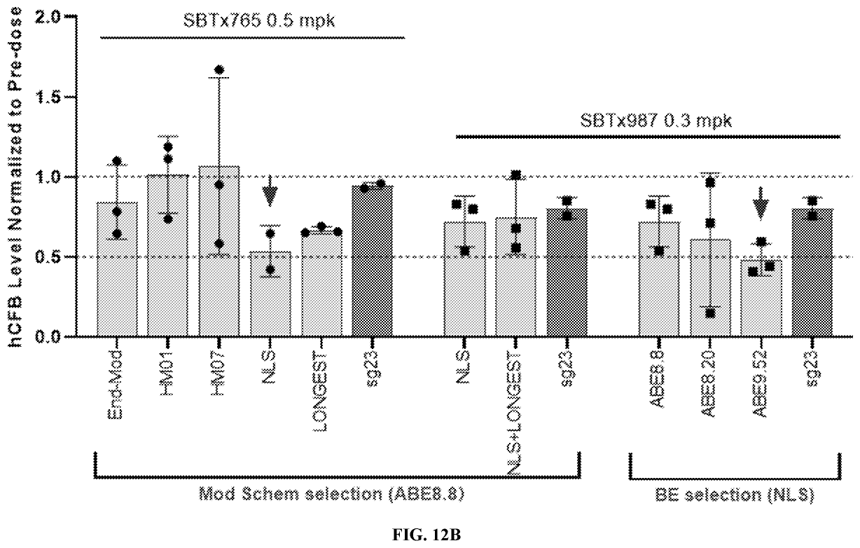

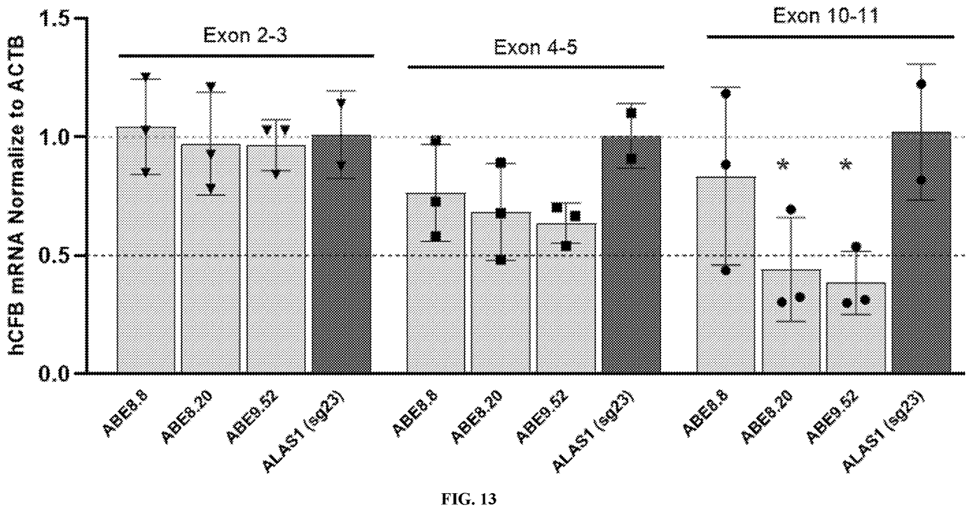

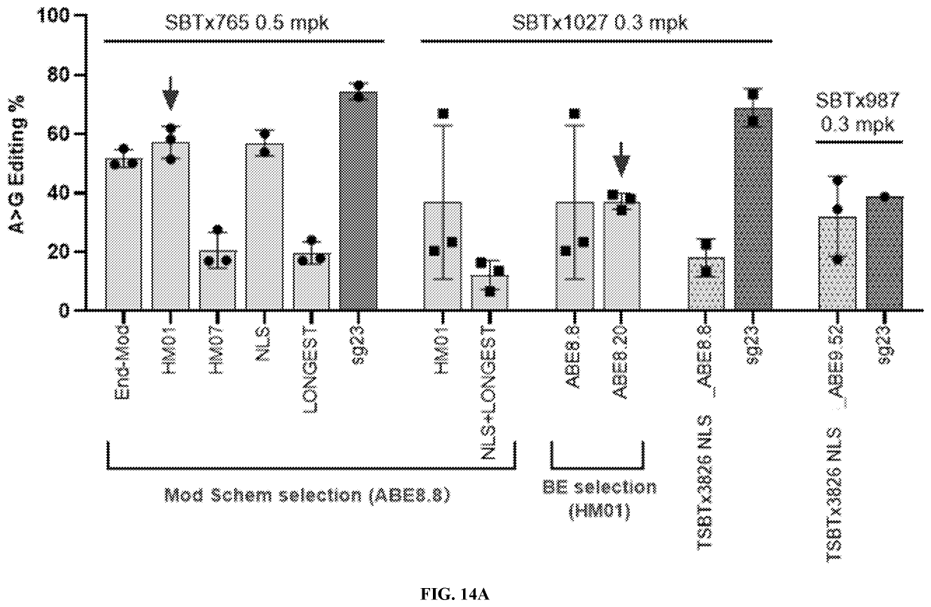

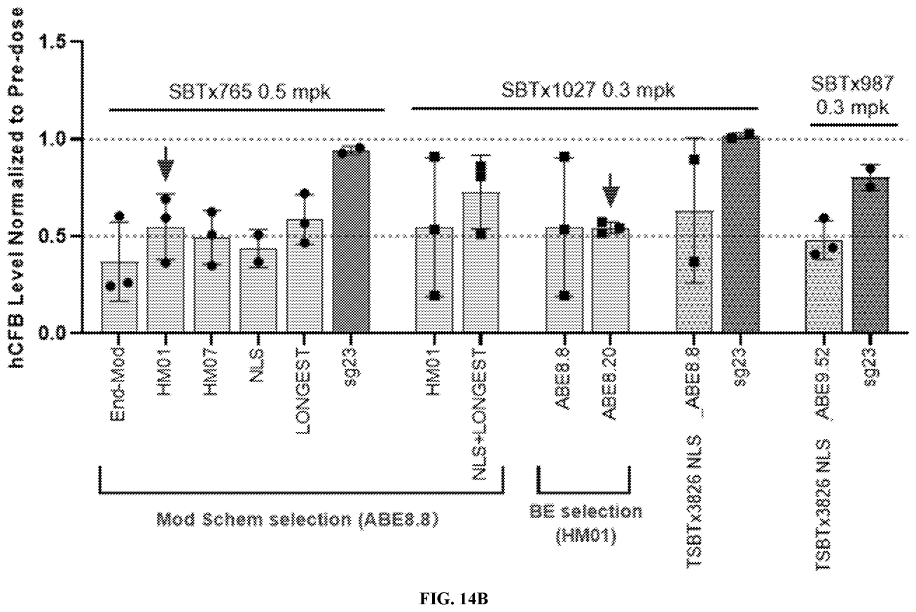

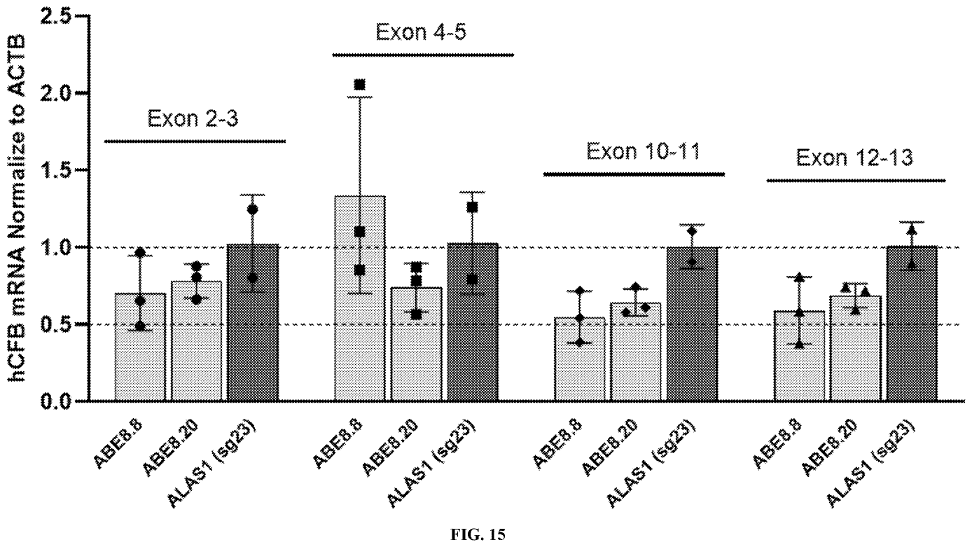

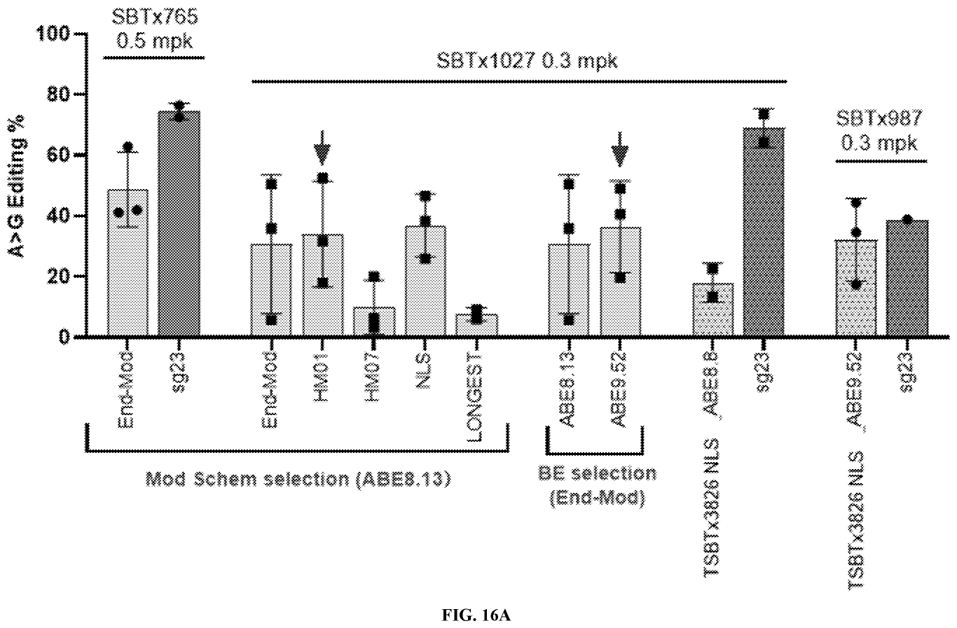

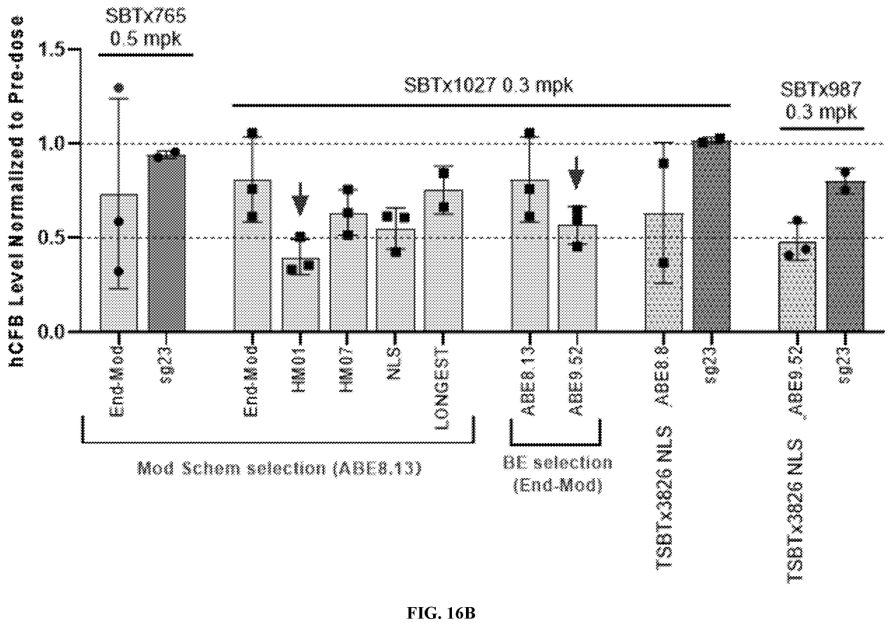

To assist in further optimizing the base editor systems, experiments were undertaken to determine the impact of guide polynucleotide nucleotide modification schemes and base editor selection on base editing in FRG™ liver-humanized mice (FIGS. 12A, 12B, 13, 14A, 14B, 15, 16A, 16B, and 17). FRG™ liver-humanized mice were administered lipid nanoparticles containing a base editor system containing a TSBTx3826 (FIGS. 12A, 12B, and 13), TSBTx3837 (FIGS. 14A, 14B, and 15), or TSBTx3835 (FIGS. 16A, 16B, and 17) guide polynucleotide with a nucleotide modification scheme selected from End-Mod, HM01, HM07, NLS, and Longest, and an mRNA encoding an adenosine base editor selected from ABE8.8, ABE8.20, and ABE8.20 with a TadA*8.20 adenosine deaminase domain containing the alterations V82T, Y147T, and Q154S. The mice were administered either 0.5 mpk or 0.3 mpk of the guide polynucleotides. Following administration of the base editor systems, percents CFB A to G editing, human CFB (hCFB) polypeptide levels, and hCFB mRNA levels were measured at 14-days post-administration. The following base editor systems were associated with high percents A to G editing and reduced hCFB protein levels in tissues of the mice: TSBTx3826 guide polynucleotide with an NLS nucleotide modification scheme+ABE8.20 with a TadA*8.20 adenosine deaminase domain containing the alterations V82T, Y147T, and Q154S; TSBTx3837 guide polynucleotide with an HM01 nucleotide modification scheme+ABE8.20; TSBTx3835 guide polynucleotide with an HM01 nucleotide modification scheme+ABE8.20 with a TadA*8.20 adenosine deaminase domain containing the alterations V82T, Y147T, and Q154S. Tissue hCFB mRNA levels measured in mice administered a base editor system containing a TSBTx3826 guide polynucleotide were consistent with disruption of mRNA splicing.

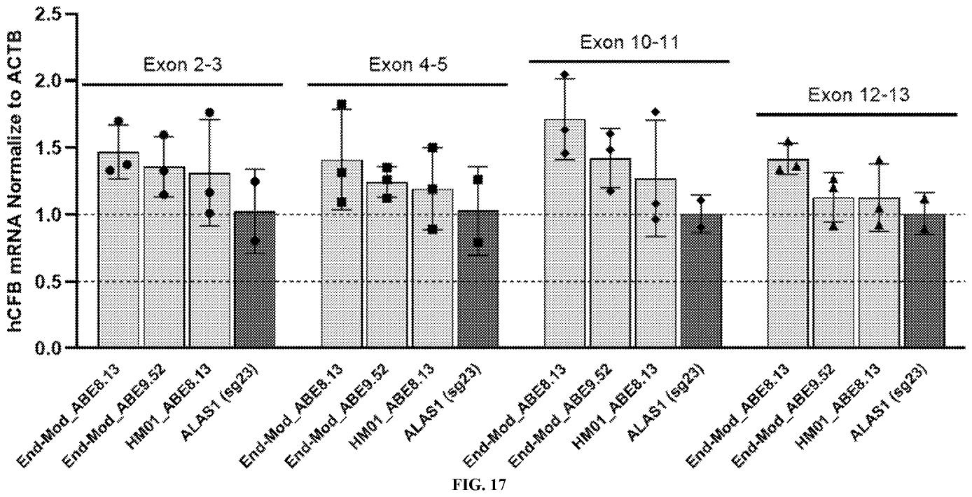

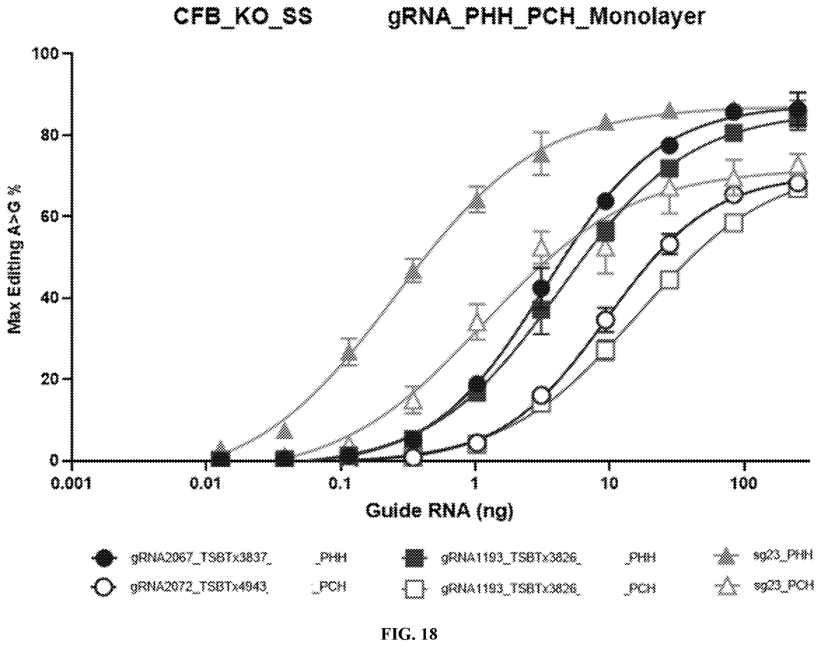

Experiments were undertaken to compare the potency (i.e., maximum percent A to G editing at a set dose of guide polynucleotide) in primary human hepatocytes (PHH) and primary cyno hepatocytes (PCH) of base editor systems containing varying doses of the guide nucleotide TSBTx3837, TSBTx4943, or TSBTx3826 (see FIG. 18 and Table 10). The PHH and PCH cells were transduced with the indicated guide polynucleotide and mRNA encoding an adenosine deaminase base editor (ABE8.8) at a mass ratio of 1-to-3 (1:3). The base editor systems were more potent in PHH than in PCH.

TABLE 10
Comparison of base editor system potency in primary human
hepatocytes (PHH) and primary cyno hepatocytes (PCH).¹¹

| | | | R- | EC-50 | |
|---|---|---|---|---|---|
| Target Site | Guide | EC-50 | square | fold change | |
| TSBTx3837 | gRNA2067 | 3.351 | 0.9967 | ~2.83x | PHH |
| | gRNA2072 | 9.477 | 0.9973 | | PCH |
| TSBTx3826 | gRNA1193 | 4.286 | 0.9956 | ~3.75x | PHH |
| | gRNA1193 | 16.07 | 0.9984 | | PCH |
| ALAS1 | sg23 | 0.2563 | 0.9929 | ~4.53x | PHH |
| | sg23 | 1.161 | 0.9722 | | PCH |

¹¹EC-50 was calculated based on guide polynucleotide (gRNA) dose. R-square indicates the R-square value for the curve fit to the data to calculate the EC-50 values.

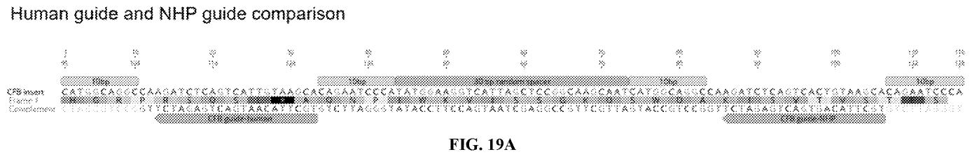

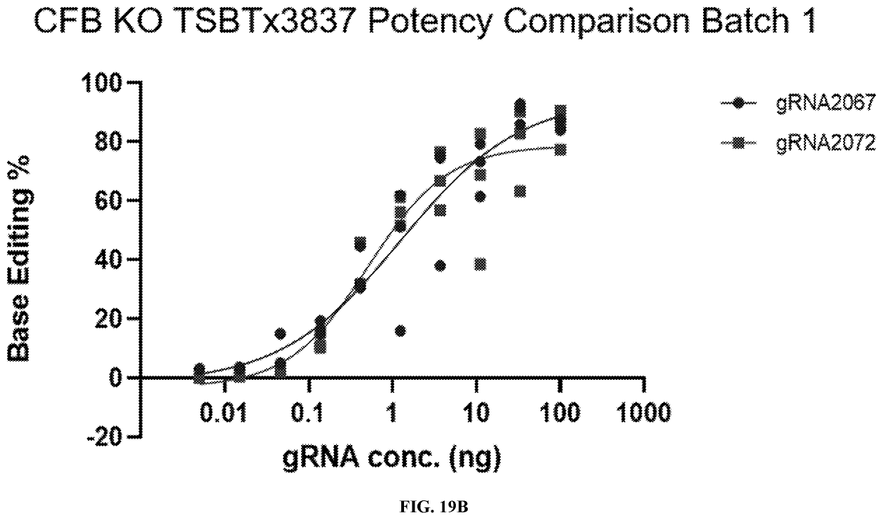

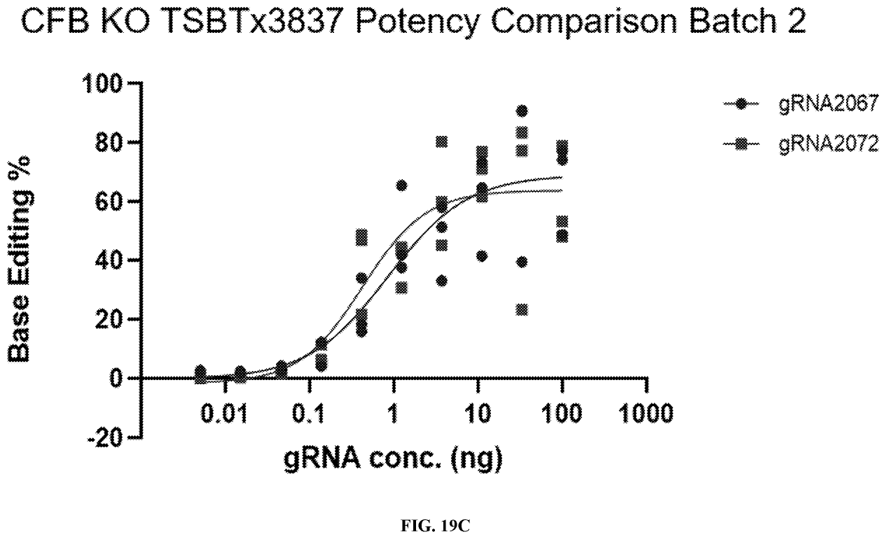

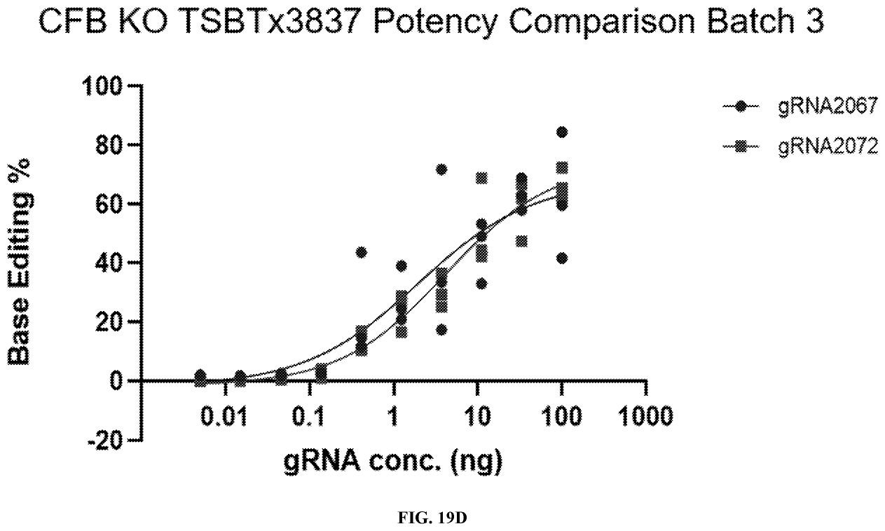

Experiments were undertaken to compare the potency (i.e., maximum percent A to G editing at a set dose of guide polynucleotide) in HEK293T cells containing the polynucleotide construct of FIG. 19A of a base editor systems containing the guide nucleotide TSBTx3837 (see FIGS. 19B-19D and Table 11). The HEK293T cells, which contained the polynucleotide construct of FIG. 19A within their genomes, were transduced with varying doses the TSBTx3837 guide polynucleotide and mRNA encoding an adenosine deaminase base editor (ABE8.8).

TABLE 11
Potency comparison of a base editor system containing a TSBTx3837 guide polynucleotide
in HEK293T cells for editing a human or non-human primate complement factor B polynucleotide.

| | | Batch 1 | | Batch 2 | | Batch 3 | |
|---|---|---|---|---|---|---|---|
| CFB | | | R- | | R- | | R- |
| (TSBTx3837) | Guide | EC50 | square | EC50 | square | EC50 | square |
| Human | gRNA2067 | 1.252 | 0.9121 | 0.8465 | 0.8331 | 1.982 | 0.8008 |
| | (TSBTx3837) | | | | | | |
| NHP | gRNA2072 | 0.5124 | 0.9242 | 0.4249 | 0.817 | 4.162 | 0.947 |
| | (TSBTx4943) | | | | | | |

Table 12 provides a summary of data gathered through the above-describe experiments relating to base editor systems containing one of three representative guide polynucleotides.

TABLE 12
Data relating to representative base editor systems.

| Guide polynucleotide | TSBTx3826 | TSBTx3837 | TSBTx3835 |
|---|---|---|---|
| Mod-Scheme | NLS | HM01 | HM01 |
| PAM sequence | NGG | NGG | NGG |
| Spacer length (nt) | 20 | 21 | 20 |
| Base Editor | 8.20 + V82T + | ABE8.20 | 8.20 + V82T + |
| | Y147T + Q154S | | Y147T + Q154S |
| Cross-reactive with a non- | Yes | No - 1nt difference | Yes |
| human primate CFB | | (no potency difference | |
| polynucleotide? | | when using NHP | |
| | | surrogate guide) | |
| Location (5′ or 3′ indicates | Exon 10-5′ | Exon 11-3′ | Exon 16-3′ |
| the position of the target site | | | |
| relative to the indicated | | | |
| Exon) | | | |
| Base Editing Percent (0.3 | 32.82% | 37.32% | 36.42% |
| mpk dose of guide | (sg23@38.8%) | (sg23@68.92%) | (sg23@68.92%) |
| polynucleotide) | | | |
| Protein Level Reduction | 48.2% (sg23 target | 54.5% (sg23 target | 56.8% (sg23 target |
| Post-Editing Relative to | polynucleotide-encoded | polynucleotide- | polynucleotide-encoded |
| Predose (0.3 mpk dose of | polypeptide @80% pre- | encoded polypeptide | polypeptide @102% |
| guide polynucleotide) | dose level) | @102% pre-dose level) | pre-dose level) |
| Ratio of terminal CFB | 38.40% | 64.30% | 113% |
| mRNA levels to sg23 target | | | |
| polynucleotide mRNA levels | | | |
| post-editing (0.3 mpk dose of | | | |
| guide polynucleotide) | | | |
| Off-Target (OT) editing (see | NLS & ABE8.8 | HM01 & ABE8.8 | End-Mod & ABE8.13 |
| Example 4) | very low editing | No OT editing risk | No OT editing risk |
| | frequency of OT gene | identified | identified |
| | HCK, Src family of | | |
| | tyrosine kinases | | |

### Example 2: Base Editing of a Complement Factor B (CFB) Polynucleotide to Disrupt a TATA Box or Alter a Translation Start Codon (AUG)

Experiments were undertaken to develop base editor systems for altering a start codon or TATA box of a CFB polynucleotide to reduce levels of functional CFB polypeptides in a cell. TATA Box, also called the Goldberg-Hogness box is a DNA sequence found in the core promoter region of a gene. It is considered a non-coding DNA sequence and important to regulating gene transcription. The base editor systems were evaluated in vitro.

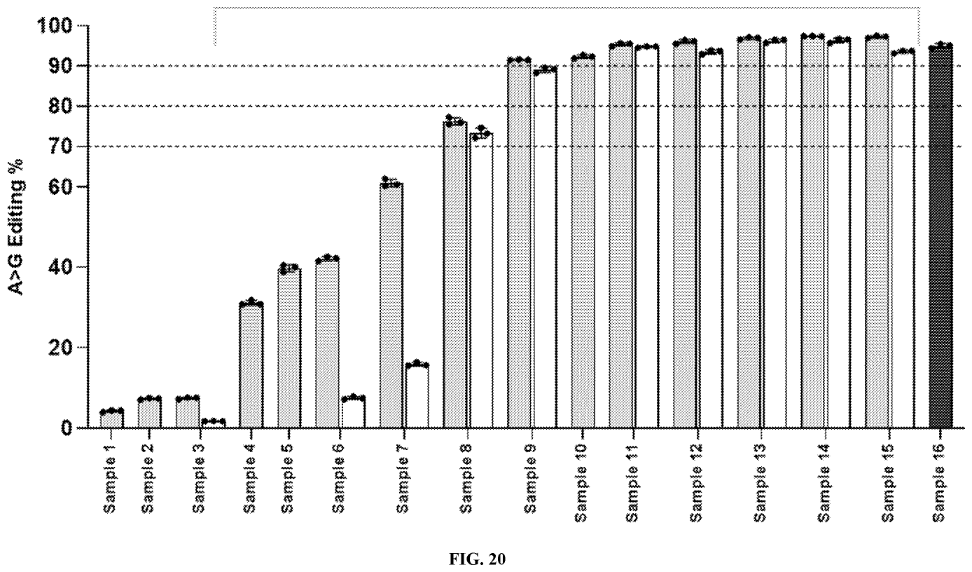

Base editor systems were developed for altering a TATA box of a CFB polynucleotide and evaluated using HepG2 cells. A predicted TATA Box corresponding to positions −157 to −151 of the CFB gene relative to start Codon was targeted for editing. The base editor systems contained mRNA encoding an adenosine deaminase base editor (ABE_NGG_20 nt_3-9_002 (abe8.8); NGA IBE16 ABE8.20 SpCas9; NGG IBE16 ABE8.20 SpCas9; NGC ABE_8.20_IBE16_SpCas9; ABE_NNNRRT_21 nt_5-14_014; ABE_NGA_20 nt_3-9_005; or ABE_8.20_SpCas9_NGC-002, where, throughout the Examples, the terms “NGG,” “NGA,” “NGC,” “NNNRRT,” and “NNGRRT” indicate the PAM binding specificity of the napDNAbp of each base editor, “IBE16” indicates a base editor with the adenosine deaminase domain inserted within the napDNAbp domain, “ABE8.8” indicates an ABE8.8 base editor, “21 nt” or “20 nt” indicates the length of the spacer of the guide polynucleotide used in combination with the base editor, “8.20” indicates a base editor containing a TadA*8.20 adenosine deaminase domain, and “SpCas9” indicates an SpCas9 napDNAbp domain) and a guide RNA targeting a TATA box of a CFB polynucleotide for base editing. Ten (10) of the base editor systems contained the same guide RNA but mRNA encoding a different base editor. The base editor systems corresponding to Sample 1 through Sample 16 of FIG. 20 are also listed in Table 12.1A.

TABLE 12.1A
Base editor systems used to alter a TATA box in HepG2 cells.

| Samples | gRNA # | mRNA # | Base Editor Name | Template Plasmid |
|---|---|---|---|---|
| Sample 1 | gRNA3654 | mRNA2743 | ABE8.20_NGC | pST-107 |
| Sample 2 | gRNA3656 | MRNA1021 | ABE_8.8_NNNRRT | pMSP985 |
| Sample 3 | gRNA3651 | mRNA3171 | ABE8.8_NGG | pMSP546 |
| | | mRNA3169 | ABE8.20_IBE16_NGG | pCR-007 |
| Sample 4 | gRNA3652 | mRNA1992 | VRQR spCas9-ABE8.8_NGA | pMSP981 |
| Sample 5 | gRNA3655 | MRNA1021 | ABE_8.8_NNNRRT | pMSP985 |
| Sample 6 | gRNA3649 | mRNA3169 | ABE8.20_IBE16_NGG | pCR-007 |
| | | mRNA3171 | ABE8.8_NGG | pMSP546 |
| Sample 7 | gRNA3648 | mRNA3169 | ABE8.20_IBE16_NGG | pCR-007 |
| | | mRNA3171 | ABE8.8_NGG | pMSP546 |
| Sample 8 | gRNA3647 | mRNA3171 | ABE8.8_NGG | pMSP546 |
| | | mRNA3169 | ABE8.20_IBE16_NGG | DCR-007 |
| Sample 9 | gRNA3642 | mRNA3171 | ABE8.8_NGG | pMSP546 |
| | | mRNA3169 | ABE8.20_IBE16_NGG | pCR-007 |
| Sample 10 | gRNA3653 | mRNA2743 | ABE8.20_NGC | PST-107 |
| Sample 11 | gRNA3650 | mRNA3171 | ABE8.8_NGG | pMSP546 |
| | | mRNA3169 | ABE8.20_IBE16_NGG | pCR-007 |
| Sample 12 | gRNA3643 | mRNA3169 | ABE8.20_IBE16_NGG | pCR-007 |
| | | mRNA3171 | ABE8.8_NGG | pMSP546 |
| Sample 13 | gRNA3645 | mRNA3171 | ABE8.8_NGG | pMSP546 |
| | | mRNA3169 | ABE8.20_IBE16_NGG | DCR-007 |
| Sample 14 | gRNA3644 | mRNA3169 | ABE8.20_IBE16_NGG | pCR-007 |
| | | mRNA3171 | ABE8.8_NGG | MSP546 |
| Sample 15 | gRNA3646 | mRNA3171 | ABE8.8_NGG | pMSP546 |
| | | mRNA3169 | ABE8.20_IBE16_NGG | pCR-007 |
| Sample 16 | sgRNA_088 | mRNA3171 | ABE8.8_NGG | pMSP546 |

The HepG2 cells were plated on Day 1 of the experiment. Media was changed on the 2^{nd} day followed by transfection with the base editor systems using LIPOFECTAMINE® MESSANGERMAX®. The HepG2 cells were administered 800 ng total of the guide polynucleotide (200 ng) and mRNA encoding the base editor (600 ng). Media was changed on Day 3 and the cells were taken down on Day 5. Quick Extract (QE) buffer was used for cell lysis and genomic DNA (gDNA) extraction. A library was generated and sequenced using next-generation sequencing (NGS) technology.

Twelve (12) of the base editor systems of Table 12.1A were associated with maximum percents A to G base editing of greater than 30% (FIG. 20). The CFB TATA box is located at positions −157 to −151 relative to the CFB polynucleotide start codon. The TATA box nucleotide positions relative to the CFB polynucleotide start codon targeted for base editing by the guide polynucleotides were as follows: gRNA3643: −156; −154; gRNA3644: −156, −154; gRNA3645: −156; −154; gRNA3646: −156; −154; gRNA3647: −154; −153; 152; gRNA3648: −154; −152; −151; gRNA3649: −154; −153; −152; −151; gRNA3650: −155; −157; gRNA3652: −154; −151; gRNA3655: −156; −154. The guide polynucleotides each targeted multiple TATA box sites for base editing. Out of 15 CFB TATA Box gRNAs assessed, 12 showed >30% editing and were further assessed for base editing in Primary Human Hepatocytes (PHH).

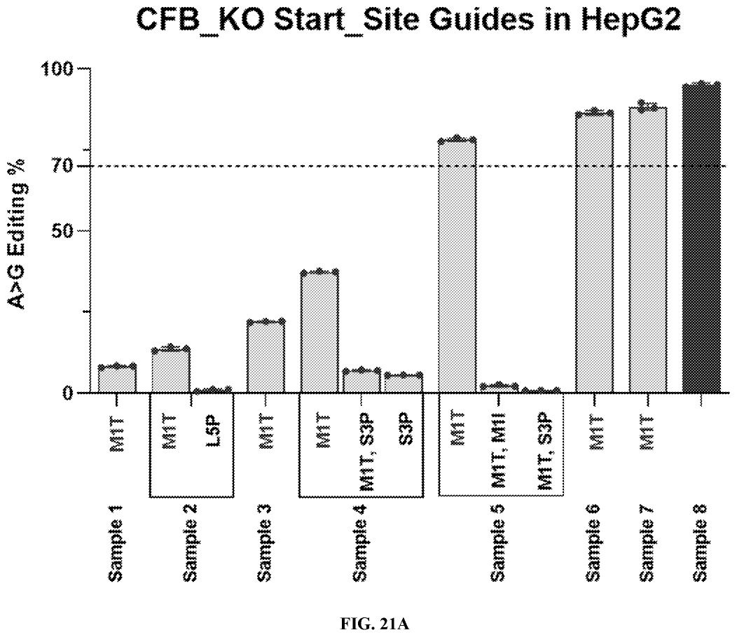

Base editor systems were developed for altering the start codon of a CFB polynucleotide and evaluated using HepG2 cells (FIG. 21A) and in a primary human hepatocyte (PHH) monolayer (FIG. 21B). The base editor systems contained a adenosine deaminase base editor (ABE_NGG_20 nt_3-9_002 (abe8.8); NGA IBE16 ABE8.20 SpCas9; NGG IBE16 ABE8.20 SpCas9; NGC ABE_8.20_IBE16_SpCas9; ABE_NNNRRT_21 nt_5-14_014; ABE_NGA_20 nt_3-9_005; or ABE_8.20_SpCas9_NGC-002) and a guide polynucleotides targeting the start codon of a CFB polynucleotide for base editing. The base editor systems corresponding to Sample 1 through Sample 8 of FIG. 21A and Sample 1 through Sample 3 of FIG. 21B are also listed in Table 12.1B.

The HepG2 cells were plated on Day 1 of the experiment. Media was changed on the 2^{nd} day followed by transfection with the base editor systems using LIPOFECTAMINE® MESSANGERMAX®. The cells were administered 800 ng total of the guide polynucleotide (200 ng) and mRNA encoding the base editor (600 ng). Media was changed on Day 3 and the cells were taken down on Day 5. Quick Extract (QE) buffer was used for cell lysis and genomic DNA (gDNA) extraction. A library was generated and sequenced using next-generation sequencing (NGS) technology. The guide polynucleotides gRNA3658 and gRNA3660 were both associated with percent A to G base editing rates of greater than 70% in the HepG2 cells (FIG. 21A).

TABLE 12.1B
Base editor systems used to alter a start codon box in HepG2 cells and PHH monoculture.

| | | | | | Template |
|---|---|---|---|---|---|
| | Samples | gRNA # | mRNA# | Base Editor Name | Plasmid |
| CFB_KO Start | Sample 1 | gRNA3661 | mRNA1992 | VRQR spCas9-ABE8.8_NGA | pMSP981 |
| Site Guides in | Sample 2 | gRNA3659 | MRNA3169 | ABE8.20_IBE16_NGG | pCR-007 |
| HepG2 | Sample 3 | gRNA3662 | mRNA3165 | ABE8.20_IBE16_NGA | pCR-001 |
| | Sample 4 | gRNA3661 | mRNA3165 | ABE8.20_IBE16_NGA | pCR-001 |
| | Sample 5 | gRNA3660 | mRNA3165 | ABE8.20_IBE16_NGA | pCR-001 |
| | Sample 6 | gRNA3658 | MRNA3169 | ABE8.20_IBE16_NGG | pCR-007 |
| | Sample 7 | gRNA3660 | mRNA1992 | VRQR spCas9-ABE8.8_NGA | pMSP981 |
| | Sample 8 | sgRNA_088 | mRNA3171 | ABE8.8_NGG | pMSP546 |
| CFB_KO Start | Sample 1 | gRNA3657 | mRNA3167 | ABE8.20_IBE16_NGC | pCR-004 |
| Site gRNA3657 in | Sample 2 | gRNA3657 | mRNA2743 | ABE8.20_NGC | pST-107 |
| PHH | Sample 3 | sgRNA_088 | mRNA3171 | ABE8.8_NGG | pMSP546 |

The primary human hepatocyte (PHH) cells were seeded in the morning and transfected with the base editor systems 4 hours after seeding. The cells were transfected with the base editor systems of Table 12.1B using LIPOFECTAMINE® MESSANGERMAX®. Media was changed on the next day. The cells were taken down on Day 4 post-seeding. Quick Extract (QE) buffer was used for cell lysis and genomic DNA (gDNA) extraction. A library was generated and sequenced using the NGS technology. The data was analyzed and graphed (FIG. 21B). The base editor system containing gRNA3657 and the base editor encoded by mRNA2743 yielded on-target editing and a low frequency of bystander edits in the PHH monolayer.

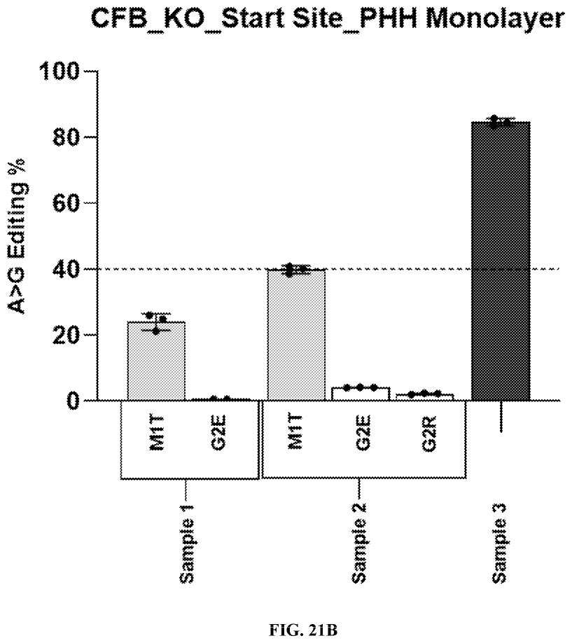

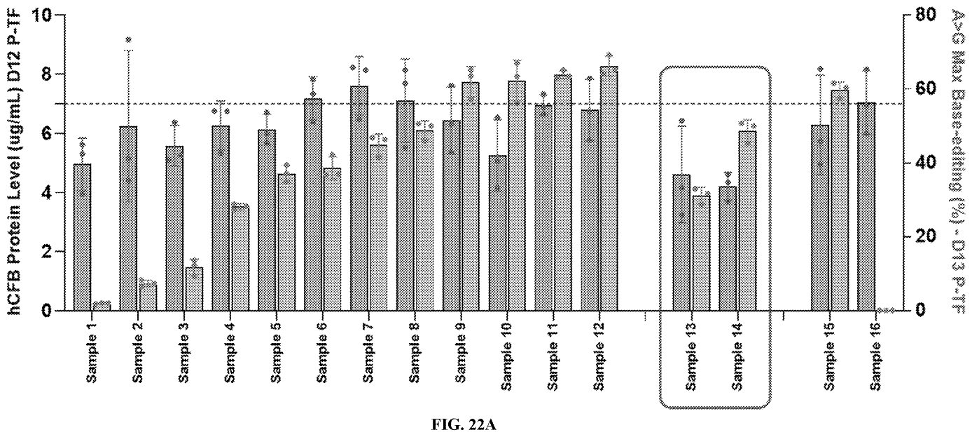

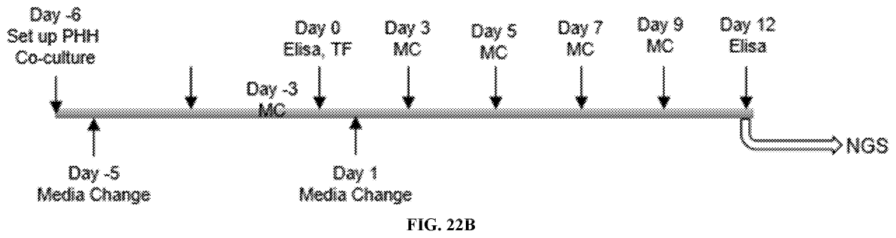

Base editor systems that performed well in the above-described experiments for editing the CFB polynucleotide TATA box or start codon were further evaluated using a primary human hepatocyte (PHH) co-culture system to assess base editing rates and the effect of base editing on secreted CFB protein levels in supernatant (FIGS. 22A and 22B). Maximum percents CFB polynucleotide A to G base editing and human CFB (hCFB) protein levels were measured in PHH transfected with base editor systems containing mRNA encoding an adenosine deaminase base editor (ABE_NGG_20 nt_3-9_002 (abe8.8); NGA IBE16 ABE8.20 SpCas9; NGG IBE16 ABE8.20 SpCas9; NGC ABE_8.20_IBE16_SpCas9; ABE_NNNRRT_21 nt_5-14_014; ABE_NGA_20 nt_3-9_005; or ABE_8.20_SpCas9_NGC-002) and a guide polynucleotide targeting the CFB polynucleotide TATA box or start codon for base editing. The base editor systems corresponding to Sample 1 through Sample 16 of FIG. 22A are also listed in Table 12.1C. Sample 16 was a negative control that did not contain any base editor system components. The PHH were co-cultured with 3T3-J2 mouse feeder cells.

TABLE 12.1C
Base editor systems used for function and editing validation in PHH co-culture.

| Samples | gRNA # | mRNA# | Base Editor Name | Template Plasmid |
|---|---|---|---|---|
| Sample 1 | gRNA3649 | mRNA3169 | ABE8.20_IBE16_NGG | pCR-007 |
| Sample 2 | gRNA3648 | mRNA3169 | ABE8.20_IBE16_NGG | pCR-007 |
| Sample 3 | gRNA3655 | MRNA1021 | ABE_8.8_NNNRRT | pMSP985 |
| Sample 4 | gRNA3647 | mRNA3171 | ABE8.8_NGG | pMSP546 |
| Sample 5 | gRNA3642 | mRNA3171 | ABE8.8_NGG | pMSP546 |
| Sample 6 | gRNA3653 | mRNA2743 | ABE8.20_NGC | pST-107 |
| Sample 7 | gRNA3650 | mRNA3171 | ABE8.8_NGG | pMSP546 |
| Sample 8 | gRNA3643 | mRNA3171 | ABE8.8_NGG | pMSP546 |
| Sample 9 | gRNA3645 | mRNA3171 | ABE8.8_NGG | pMSP546 |
| Sample 10 | gRNA3652 | mRNA1992 | VRQR spCas9-ABE8.8_NGA | pMSP981 |
| Sample 11 | gRNA3646 | mRNA3171 | ABE8.8_NGG | pMSP546 |
| Sample 12 | gRNA3644 | mRNA3171 | ABE8.8_NGG | pMSP546 |
| Sample 13 | gRNA3658 | mRNA3169 | ABE8.20_IBE16_NGG | pCR-007 |
| Sample 14 | gRNA3660 | mRNA1992 | VRQR spCas9-ABE8.8_NGA | pMSP981 |
| Sample 15 | sgRNA_088 | mRNA3171 | ABE8.8_NGG | pMSP546 |
| Sample 16 | N/A | N/A | N/A | N/A |

The PHH were seeded on Day −6 and the feeder cells were added on Day −5 to form the co-culture system. Media was changed on the next day. On Day-3 to Day 0 (72 hours) media was collected as a baseline sample for ELISA (enzyme-linked immunosorbent assay) and the PHH were transfected on Day 0. Transfection with phosphate buffered saline (PBS) was used as a negative control (Sample 16). A base editor system targeting ALAS1 (Sample 15) for editing was used as a positive editing control and negative protein reduction control. The cells were transfected with the base editor systems using LIPOFECTAMINE® MESSANGERMAX®. Media was changed on the next day and then every 2-3 days. Supernatant was collected from the last 72 hours of the culture for ELISA. The ELISA results (FIG. 22A) were normalized to the pre-transfection baseline. The following kit was used for the ELISA: ab137973 Human Factor B ELISA Kit. The PHH co-culture system was taken down on Day 12. The cells were in culture for a total of 18 days. Quick Extract (QE) buffer was used for cell lysis and genomic DNA (gDNA) extraction. A library was generated and sequenced using next-generation sequencing (NGS) technology.

A number of the base editor systems of Table 12.1C were associated with high maximum percents A to G base editing and reductions in hCFB protein levels. In particular, the guides gRNA3658 and gRNA3660, which targeted the CFB polynucleotide start codon for base editing, were associated with percents maximum A to G base editing at day 13 post-transfection of greater than around 30% and with reductions in hCFB protein levels at day 12 post-transfection (FIG. 22A).

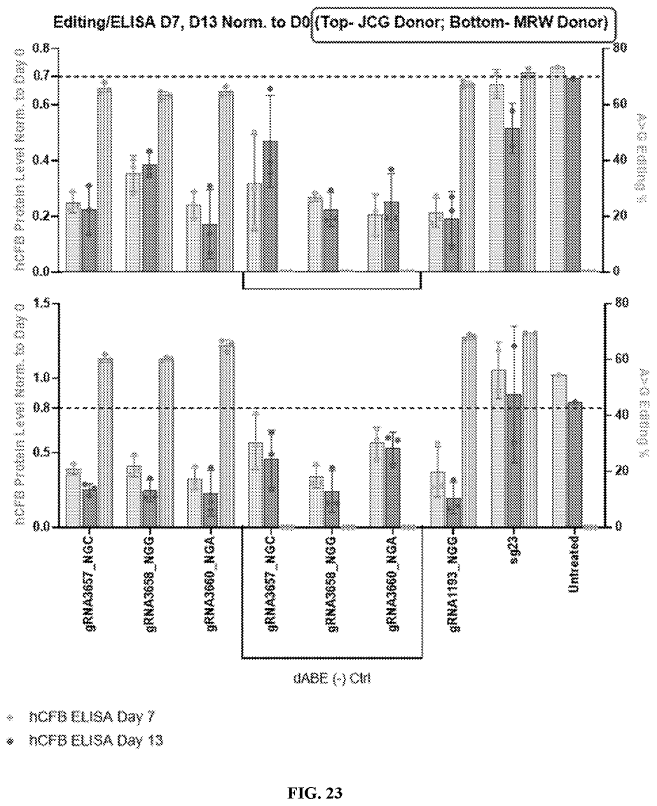

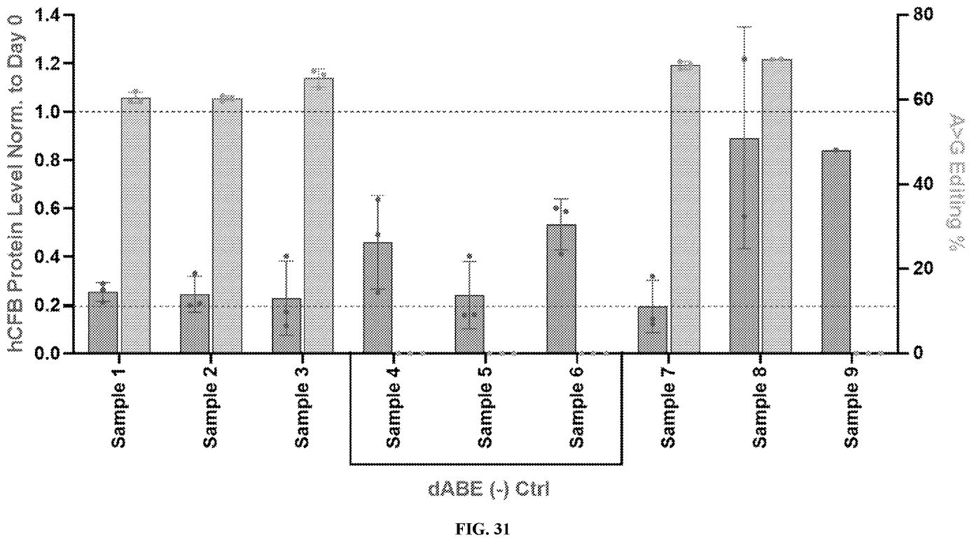

Base editor systems targeting the CFB polynucleotide start codon for base editing and containing one of the guide polynucleotides gRNA3657, gRNA3658, and gRNA3660 were further evaluated in primary human hepatocyte (PHH) co-cultures (FIGS. 23 and 31). Percents CFB polynucleotide A to G base editing and human CFB (hCFB) protein levels were measured in PHH co-cultures transfected with base editor systems containing mRNA encoding an adenosine deaminase base editor (ABE_NGG_20 nt_3-9_002 (abe8.8); NGG IBE16 ABE8.20 SpCas9; NGC ABE_8.20_IBE16_SpCas9; ABE_8.20_SpCas9_NGC-002; pHRB-445_From_pHRB-438 E59A, N108D-001; pHRB-446_From_pHRB-439-001; pHRB-449_From_pHRB-442-001) with specificity for an NGC, NGG, or GNA PAM sequence, and a guide polynucleotide targeting the CFB polynucleotide start codon for base editing. The base editor systems corresponding to Sample 1 through Sample 9 of FIG. 31 are also listed in Table 12.1D. The base editor systems of Samples 4 to 6 contained mRNA encoding catalytically inactive base editor polypeptides (dead TadA) and were used as negative controls. A base editor system containing a gRNA1193 targeting a splice site for editing was used as a positive control. Sample 9 was a negative control that did not contain any base editor system components.

TABLE 12.1D
Base editor systems used for function and editing validation in PHH
co-culture. Catalytically inactive editors are shown in bold text.

| Samples | gRNA # | mRNA# | Base Editor Name | Template Plasmid |
|---|---|---|---|---|
| Sample 1 | gRNA3657 | mRNA2743 | ABE8.20_NGC | pST-107 |
| Sample 2 | gRNA3658 | mRNA3169 | ABE8.20_IBE16_NGG | pCR-007 |
| Sample 3 | gRNA3660 | mRNA1992 | VRQR spCas9-ABE8.8_NGA | pMSP981 |
| Sample 4 | gRNA3657 | mRNA3410 | dABE8.20_{—}NGC | pHRB-449_{—}From_{—}pHRB-442 |
| Sample 5 | gRNA3658 | mRNA3406 | dABE8.20_{—}NGG | pHRB-445_{—}From_{—}pHRB-438 |
| | | | | E59A, N108D |
| Sample 6 | gRNA3660 | mRNA3407 | dABE8.20_{—}NGA | pHRB-446_{—}From_{—}pHRB-439 |
| Sample 7 | gRNA1193 | mRNA3171 | ABE8.8_NGG | pMSP546 |
| Sample 8 | sgRNA_088 | mRNA3171 | ABE8.8_NGG | pMSP546 |
| Sample 9 | N/A | N/A | N/A | N/A |

The PHH co-cultures were prepared by co-culturing PHH with 3T3-J2 mouse feeder cells. The PHH were seeded on Day-6 and the feeder cells were added on Day-5 to form a co-culture system. Media was changed on the next day. On Days-3 to 0 (72 hours), media was collected as a baseline sample for ELISA and the cells were transfected with the base editor systems on Day 0. The cells were transfected with the base editor systems using LIPOFECTAMINE® MESSANGERMAX®. Transfection with phosphate buffered saline (PBS) was used as a negative control (Sample 9). The cells were administered 800 ng total of the guide polynucleotide (200 ng) and mRNA encoding the base editor (600 ng). Media was changed the day after transfection and then every 3 days. Supernatant was collected for ELISA evaluation on Days 7, 10, and 13 post-transfection. The ELISA results were normalized to pre-transfection baseline (FIGS. 23 and 31). The following kit was used for the ELISA: ab137973 Human Factor B ELISA Kit.

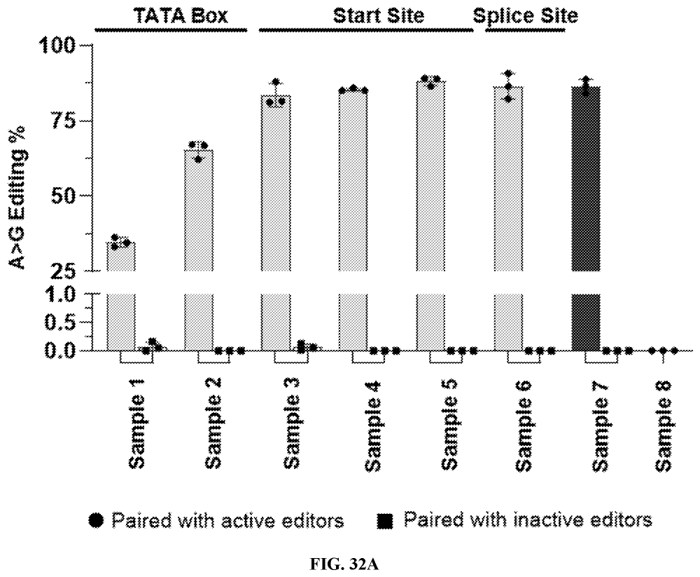

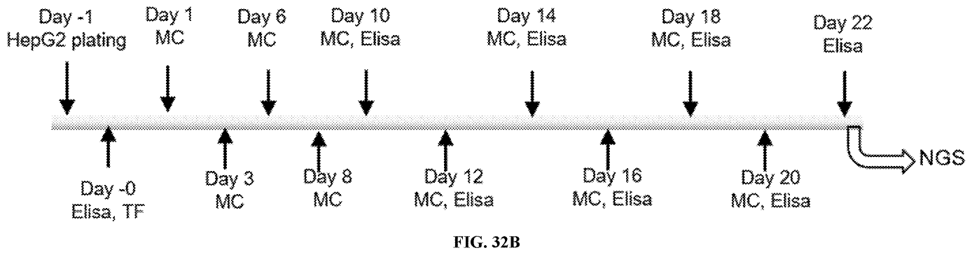

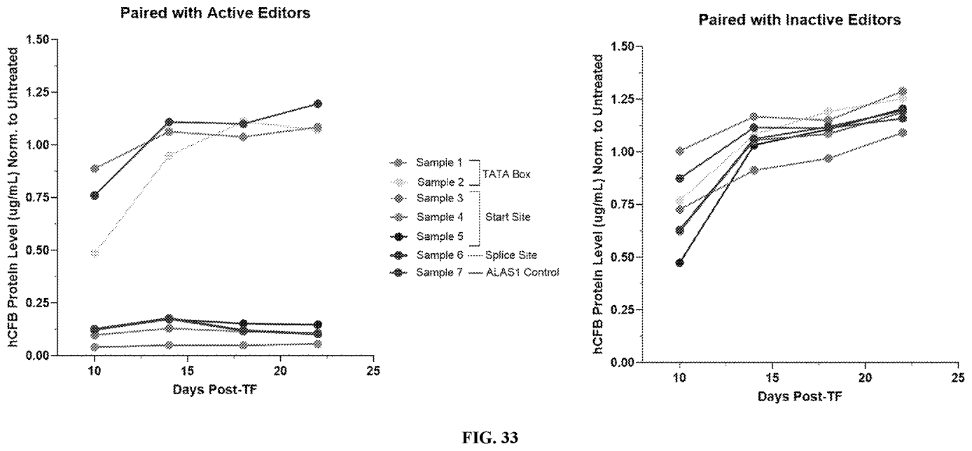

The base editor systems of Table 12.1D were associated with reductions in hCFB polypeptide levels at days 12 and 13 post-transfection. The base editor systems were also associated with base editing rates of greater than about 60%. An experiment was undertaken to demonstrate sustained reductions in hCFB protein levels following base editing of an hCFB polynucleotide start codon in a long-term (~22 days) HepG2 culture system (FIG. 32B) using the base editor systems listed in Table 12.1E and corresponding to Samples 1 to 8 (FIGS. 32A and 33). Sample 8 of Table 12.1E was a phosphate buffered saline (PBS) negative control that did not contain any component of a base editor system, and Sample 7 was a positive control base editor system targeting ALAS1 for editing. The base editor systems contained mRNA encoding a base editor together with a gRNA molecule. The cells were plated on Day-1 and transfected with the base editor systems the next day using LIPOFECTAMINE® MESSANGERMAX®. Transfection with phosphate buffered saline (PBS) was used as a negative control (Sample 8). As negative controls, the cells were also transfected with base editor systems containing mRNA encoding a catalytically inactive base editor. Media was changed every two days after the transfection. Media was collected on days 10, 14, 18, and 22 post-transfection for evaluation using ELISA (enzyme-linked immunosorbent assay). Cells were taken down 22 days post-transfection. Quick Extract (QE) buffer was used for cell lysis and genomic DNA (gDNA) extraction. A library was generated and sequenced using next-generation sequencing (NGS) technology. The following kit was used for the ELISA: ab137973 Human Factor B ELISA Kit.

TABLE 12.1E
Base editor systems used for functional assessment of start codon targeting guides
in a long-term HepG2 culture system. Catalytically inactive editors are shown in
bold text. “ABE9.52” refers to a base editor containing the following adenosine
deaminase domain: TadA*8.20 with the amino acid alterations V82T, Y147T, and Q154S.

| Guide | | | | | |
|---|---|---|---|---|---|
| Group | Samples | gRNA # | mRNA# | BE Name | Template Plasmid |
| CFB_KO | Sample | gRNA3649 | mRNA3171 | ABE8.8_NGG | pMSP546 |
| TATA Box | 1 | | mRNA3406 | dABE8.20_{—}NGG | pHRB- |
| | | | | | 445_{—}From_{—}pHRB- |
| | | | | | 438 E59A, N108D |
| | Sample | gRNA3655 | mRNA1021 | ABE_8.8_NNNRRT | pMSP985 |
| | 2 | | mRNA3409 | dABE8.20 NNNRRT | pHRB- |
| | | | | | 448_{—}From_{—}pHRB- |
| | | | | | 441 |
| CFB_KO | Sample | gRNA3657 | mRNA2743 | ABE8.20_NGC | pST-107 |
| Start Site | 3 | | mRNA3410 | dABE8.20_{—}NGC | pHRB- |
| | | | | | 449_{—}From_{—}pHRB- |
| | | | | | 442 |
| | Sample | gRNA3658 | mRNA3169 | ABE8.20_IBE16_NGG | pCR-007 |
| | 4 | | mRNA3406 | dABE8.20_{—}NGG | pHRB- |
| | | | | | 445_{—}From_{—}pHRB- |
| | | | | | 438 E59A, N108D |
| | Sample | gRNA3660 | mRNA1992 | VRQR spCas9-ABE8.8_NGA | pMSP981 |
| | 5 | | mRNA3407 | dABE8.20_{—}NGA | pHRB- |
| | | | | | 446_{—}From_{—}pHRB- |
| | | | | | 439 |
| CFB_KO | Sample | gRNA2445 | mRNA3969 | ABE9.52 | pMSP1197 |
| Splice Site | 6 | | mRNA3406 | dABE8.20_{—}NGG | pHRB- |
| | | | | | 445_{—}From_{—}pHRB- |
| | | | | | 438 E59A, N108D |
| ALAS1 | Sample | gRNA_088 | mRNA3171 | ABE8.8_NGG | pMSP546 |
| Control | 7 | | mRNA3406 | dABE8.20_{—}NGG | pHRB- |
| | | | | | 445_{—}From_{—}pHRB- |
| | | | | | 438 E59A, N108D |
| N/A | Sample | N/A | N/A | N/A | N/A |
| | 8 | | | | |

Many of the base editor systems of Table 12.1E were associated with greater than about 60% base editing rates (FIG. 32A), and all base editor systems targeting an hCFB start site for editing were associated with a lasting reduction in hCFB protein levels (FIG. 33)

### Example 3: Base Editing of a Complement Factor B (CFB) Polynucleotide to Reduce Activity of the Encoded CFB Polypeptide

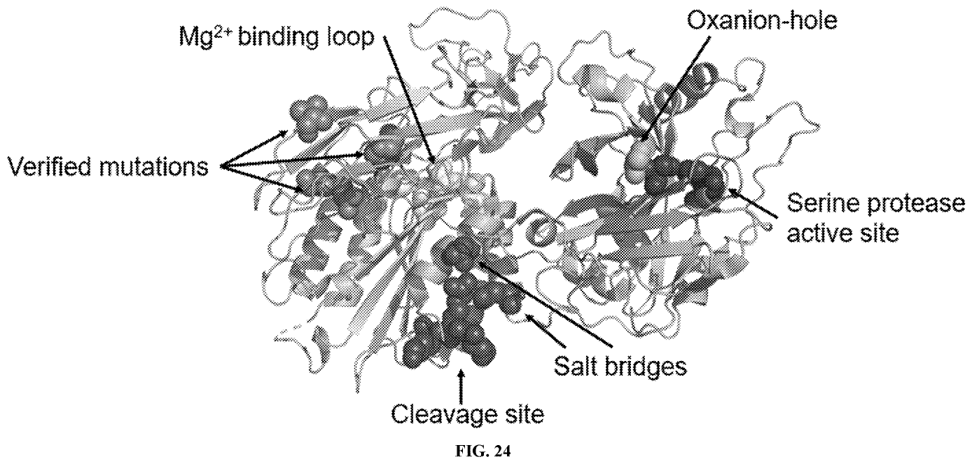

Experiments were undertaken to develop base editor systems for introducing missense mutations to the protein-coding region of a CFB polynucleotide to reduce or eliminate activity of the encoding CFB polypeptide in a cell. A structure of CFB depicting the amino acid residues of CFB targeted for alteration using base editing is provided at FIG. 24. Table 13 provides a description of the amino acid residues of CFB targeted to be altered (i.e., R259, H526, D576, S699, S278, S280, T353, P171, V177, R203, K258, K260, E471, E232, D276, D389, E255, and/or G697) together with the corresponding target nucleotide sequences. The base editor systems were evaluated in vitro.

TABLE 13
Amino acid residues of CFB targeted to be altered by base editing together
with the corresponding target sequences.
Residues

| | | |
|---|---|---|
| Tier 1 | Cleavage site, Salt bridges | R259 |
| | SP active site | H526 |
| | | D576 |
| | | S699 |
| | Mg²⁺ binding loop | S278 & S280 |
| | | T353 |
| | Verified* | P171 |
| | | V177 |
| | | R203 |
| Tier 2 | Cleavage site | K258 & K260 |
| | Salt bridges | E471 |
| | | E232 |
| | Mg²⁺ binding loop | D276 |
| | | D389 |
| | Verified* | E255 |
| | Oxyanion-hole | G697 |

*The term “verified” means an alteration known to reduce activity of CFB.

| | Target Site Sequence (target codon(s) | SEQ ID | |
|---|---|---|---|
| Residues | are shown in bold) | NO | by Base Editing |
| R259 | GGGGAACAACAGAAGCGGAAGATCGTCCTG | 3798 | R259W, R259Q |
| H526 | GTGCTGACAGCAGCACATTGTTTCACTGTG | 3799 | H526Y, H529R, |
| | | | H529Q |
| D576 | GAATTTTATGACTATGACGTTGCCCTGATC | 3800 | D576N, D576G |
| S699 | CTCCTTGCAGGTGATTCTGGCGGCCCCTTG | 3801 | S699P, S699F |
| S278 & | GTGCTAGATGGATCAGACAGCATTGGGGCCAGC | 3802 | S278T, S278L, |
| S280 | | | S280G, S280N |
| T353 | AAGTTGAAGTCAGGGACTAACACCAAGAAG | 3803 | T353A, T353I |
| P171 | TCCAACCCGGGCATCCCCATTGGCACAAGG | 3804 | P171S, P171L |
| V177 | ATTGGCACAAGGAAGGTGGGCAGCCAGTAC | 3805 | V177M (also start |
| | | | another fragment), |
| | | | V177A |
| R203 | CGTGGCTCCCAGCGGCGAACGTGTCAGGAA | 3806 | Early Stop Codon, |
| | | | R203Q* |
| K258 & | GGGGAACAACAGAAGCGGAAGATCGTCCTG | 3807 | K258E, K258R, |
| K260 | | | K260E, K260R |
| E471 | CCTTCTCTGCCAGATGAAAGCCAGTCTCTG | 3808 | E471K, E471A |
| E232 | CCTCAAGAGGTGGCCGAAGCTTTCCTGTCT | 3809 | E232K, E232A |
| D276 | ATCTACCTGGTGCTAGATGGATCAGACAGC | 3810 | D276N, D276G |
| D389 | ATCATCCTCATGACTGATGGTCAGAAGGGA | 3811 | D389N, D389G |
| E255 | TCCTGGCACCCAGGGGAACAACAGAAGCGG | 3812 | E255K, E255A |
| G697 | CCCAATACTTGCAGAGGTGATTCTGGCGGC | 3813 | G697S, G697D |

*Identified in patient and result in partially cleaved protein with no ligand binding or functional activity. Listed AA # contains 25 aa signaling peptide.

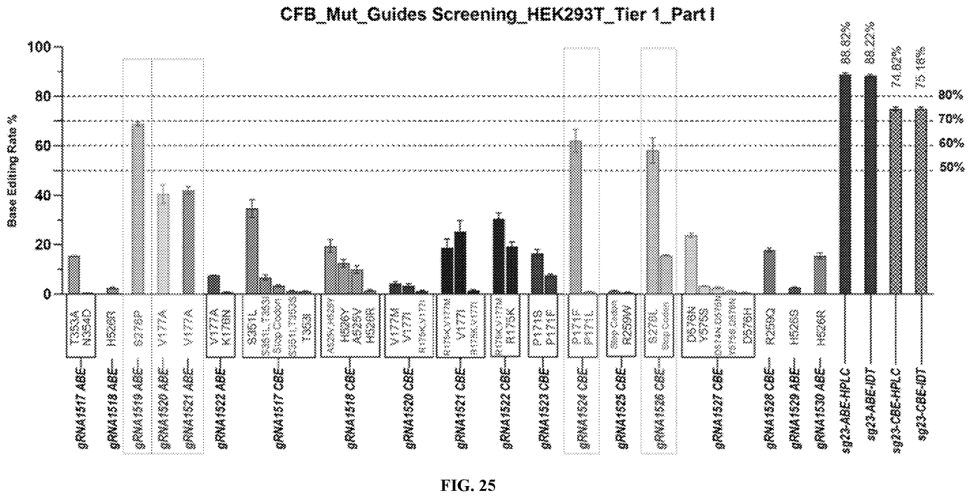

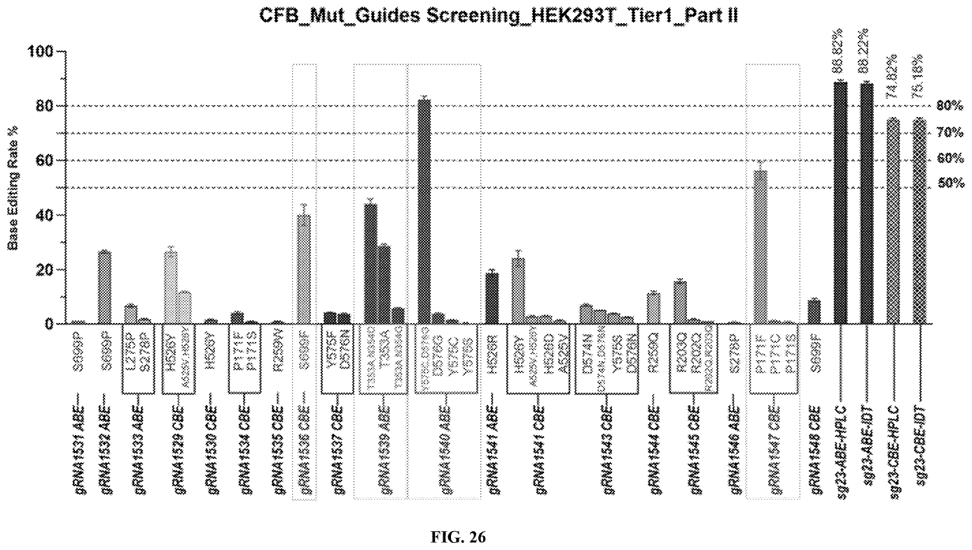

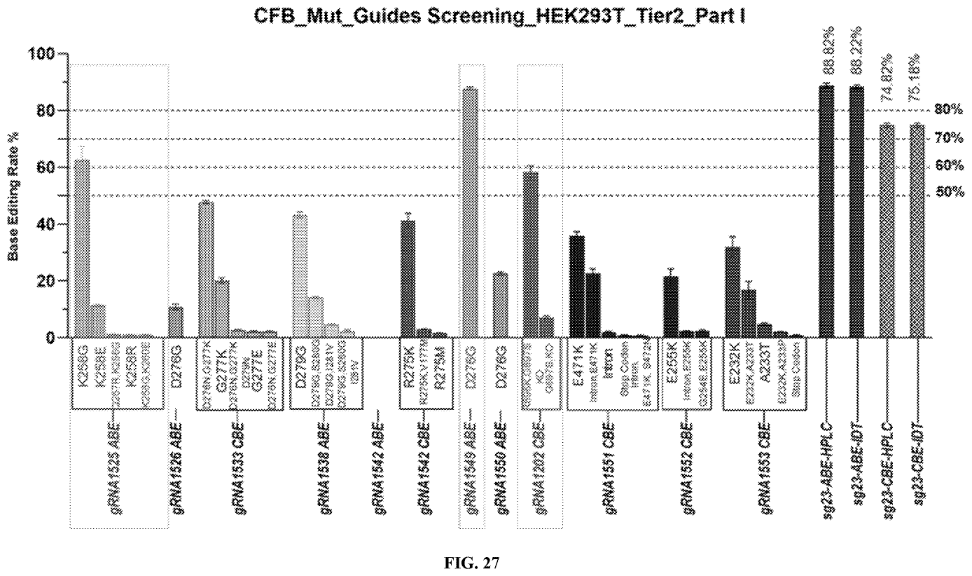

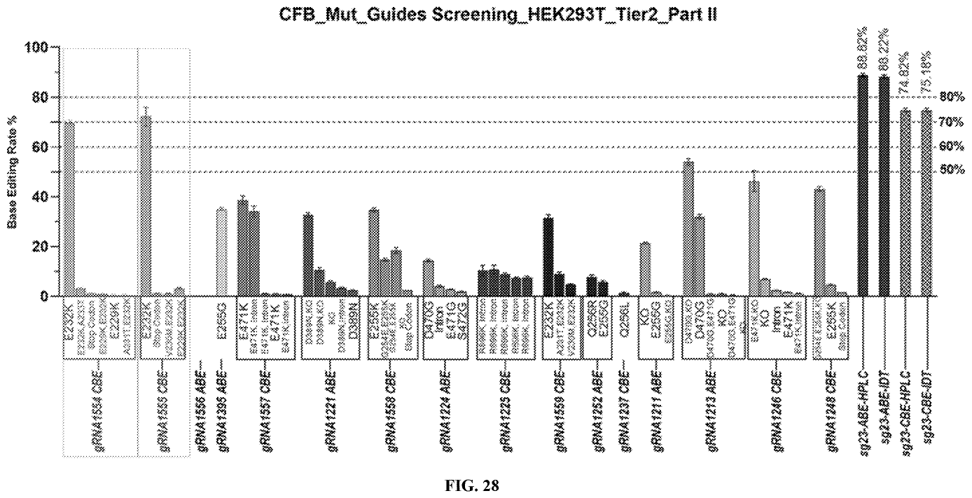

Base editing of CFB in HEK293T cells using base editor systems targeting the sites 5 listed in Table 13 was measured (FIGS. 25-28 and Table 14). The base editor systems contained a guide polynucleotide targeting a CFB polynucleotide for base editing and an adenosine deaminase base editor or a cytidine deaminase base editor (ABE_NGG 20 nt 3-9_002; CBE_NGG_20 nt_4-9_003; ABE_NGA_20 nt_3-9_005; ABE_NGC_20 nt_3-9_008; ABE_NNGRRT_21 nt_5-14_011; CBE_NNGRRT_21 nt_3-12_012; ABE_NNNRRT_21 nt_5-14_014; CBE_NNNRRT_21 nt_3-12_015; CBE_NGG_20 nt_4-9_003; ABE_NGA_20 nt_3-9_005; CBE_NGA_20 nt_4-9_006; or CBE_NNGRRT_21 nt 3-12_012), as indicated in FIGS. 25-28. Some of the base editor systems were associated with base editing rates of over 50%.

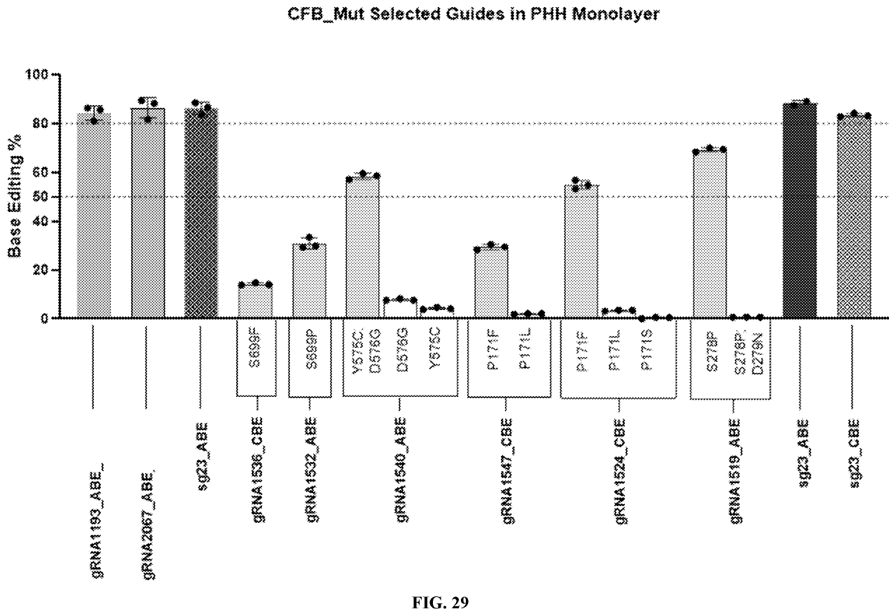

Base editor systems that performed well in the HEK293T cells were further evaluated in a primary human hepatocyte (PHH) monolayer (FIG. 29 and Table 14). The base editor systems contained a guide polynucleotide targeting a CFB polynucleotide for base editing and an adenosine deaminase base editor or a cytidine deaminase base editor, as indicated in FIG. 29, which presents a sub-portion of the data presented in FIGS. 25-28. The PHH monolayer cells were transfected with 200 ng of the guide polynucleotide and 600 ng mRNA encoding the base editor. The guide polynucleotide gRNA1540 was associated with generation of the CFB alterations Y575C and D576G with greater than 50% editing efficiency. The guide polynucleotide gRNA1524 was associated with generation of a P171F CFB alteration with greater than 50% editing efficiency. The guide polynucleotide gRNA1519 was associated with generation of a S278P CFB alteration with greater than 50% editing efficiency.

TABLE 14
CFB polynucleotide base editing rates HEK293T and PHH
monolayer cells for representative base editor systems.

| | | Editing in | Editing in | |
|---|---|---|---|---|
| | Generated | HEK293T | PHH | NHP X- |
| gRNA/editor | Mutations | (%) | Mono (%) | reactivity |
| gRNA1536_CBE | S699F | 39.98 | 14.34 | Y |
| gRNA1532_ABE | S699P | 26.28 | 30.78 | Y |
| gRNA1540_ABE | Y575C&D576G | 82.11 | 58.42 | Y |
| | D576G | | | |
| | Y575C | 3.72 | 7.8 | |
| | | 1.47 | 4.15 | |
| gRNA1547_CBE | P171F | 56.23 | 29.33 | N |
| | P171L | 0 | 1.98 | |
| gRNA1524_CBE | P171F | 62.06 | 54.9 | Y |
| | P171L | 1.05 | 3.32 | |
| | P171S | 0 | 0.6 | |
| gRNA1519_ABE | S278P | 68.78 | 69.23 | Y |
| | S278P&D279N | 0 | 0.63 | |
| Sg23_ABE | N/A | 88.22 | 88.23 | Y |
| Sg23_CBE | N/A | 75.18 | 83.38 | Y |

### Example 4: Assessment of Possible Off-Target Base Editing Mediated by Guide Polynucleotides of the Disclosure

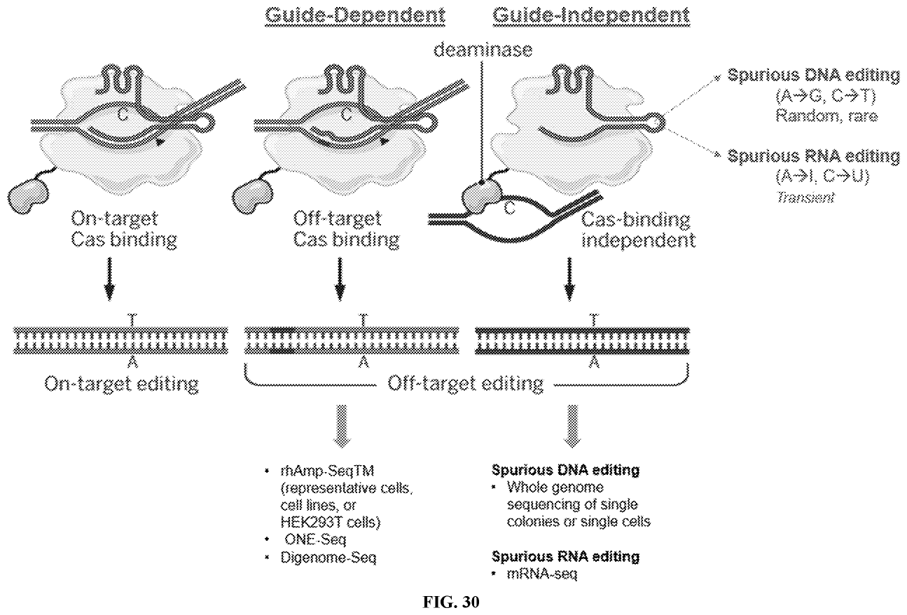

Some base editors and/or guide polynucleotides can be associated with potentially harmful guide-dependent or guide-independent off-target base editing of DNA or RNA (FIG. 30). Such off-target base editing can be detrimental to the health of a subject administered a base editor system. For example, some off-target editing could lead to a disease in a subject, such as a cancer. Accordingly, experiments were undertaken to assess off-target editing of guide polynucleotides used in the above Examples.

Off-target editing was assessed for TSBTx3826, TSBTx3835, and TXBTx3837 guide polynucleotides in silico using rhAmpSeq™ panels of less than 500 sites (Table 15). Biologically relevant guide-dependent off-target loci were nominated for evaluation for off-target editing using rhAmpSeq™ (Table 15). A comparative, non-exhaustive, guide-dependent off-target assessment of gRNA: mRNA combinations was undertaken. The following criteria were used for off-target site validation for the guide polynucleotides targeting CFB for base editing: 1) off-target site has an A>G edit in positions 4-9 of the target site that is significantly enriched in edited cells compared to untreated cells; 2) off-target edits is reproducible across replicates. Results of an assessment of off-target editing using these criteria for guide polynucleotides containing different nucleotide modification schemes is summarized in Table 16. No off-target edits were predicted to affect splicing, no off-target edits were known to be pathogenic in ClinVar, which includes cancer driver mutations, and no off-target edit was a known variant in the UK Biobank (UKBB), gnomAD, or TOPMed databases (Table 17). ClinVar is an NIH public archive of human genetic variation and associated phenotypes with supporting evidence. UKBB is a database of comprehensive medical record and genetic data from 500 k individuals in the United Kingdom. The database gnomAD contains a collection of exome and genome sequences (~140 k) to enable calculation of each gene's loss of function tolerance. The TOPMed database integrates-omics data with molecular, behavioral, imaging, environmental, and clinical data to improve the prevention and treatment of heart, lung, blood, and sleep disorders.

TABLE 15
CFB rhAmpSeq panels

| | Sites | Sites |
|---|---|---|
| Target site | Nominated | Assessed |
| TSBTx3826 | 335 | 312 (93%) |
| TSBTx3835 | 284 | 251 (88%) |
| TSBTx3837 | 268 | 240 (90%) |

TABLE 16
Off-target base editing assessment summary.

| | | Total | | | | |
|---|---|---|---|---|---|---|
| | | number | | | Associated | |
| | gRNA | of off- | | | Gene in | |
| | mod | target | Missense | | Cancer | Max edit |
| Target Site | sceme | sites | Variants | Pathogenic | Census | frequency |
| TSBTx3826 | End Mod | 1 | 0 | 0 | 0 | 9.51 |
| TSBTx3826 | HM01 | 1 | 0 | 0 | 0 | 7.2 |
| TSBTx3826 | HM07 | 2 | 0 | 0 | 0 | 32.99 |
| TSBTx3826 | NLS | 2 | 1 | 0 | 0 | 8.57 |
| TSBTx3826 | LONGEST | 1 | 0 | 0 | 0 | 10.77 |
| TSBTx3835 | End Mod | 0 | 0 | 0 | 0 | 0 |
| TSBTx3837 | End Mod | 0 | 0 | 0 | 0 | 0 |
| TSBTx3837 | HM01 | 0 | 0 | 0 | 0 | 0 |
| TSBTx3837 | HM07 | 1 | 0 | 0 | 0 | 0.36 |
| TSBTx3837 | NLS | 0 | 0 | 0 | 0 | 0 |
| TSBTx3837 | LONGEST | 0 | 0 | 0 | 0 | 0 |

TABLE 17
Off-target base editing assessment summary.

| | | | | % Off-target editing | | | | |
|---|---|---|---|---|---|---|---|---|
| | Genomic | Off-target | | (Lipofection) | | | | |
| Target Site | Coordinates | gene | Consequence | End Mod | HM01 | HM07 | NLS | LONGEST |
| TSBTx3826 | chr20:32088617- | HCK | Non- | . | . | . | 0.20 | . |
| | 32088640(−) | | synonymous | | | | | |
| | chrX:41238829- | . | Intergenic | 9.51 | 7.20 | 32.99 | 7.57 | 10.77 |
| | 41238852(+) | | | | | | | |
| | chr18:72730463- | . | Intergenic | . | . | 2.22 | . | . |
| | 72730485(−) | | | | | | | |
| TSBTx3837 | chr10:29828002- | . | Intergenic | HM07: 0.36 | | | | |
| | 29828024(−) | | | | | | | |

| | | | Primary | Gene | |
|---|---|---|---|---|---|
| | Gene expressed | Off-target | Disease | Tolerant to | |
| | in Liver/Spleen | in | Association of | Loss of | Gene |
| Target Site | (TPM ≥ 1) | Enhancer | Gene | Function | Description |
| TSBTx3826 | Liver, Spleen | n | Cancer | y | Src family of |
| | | | | | tyrosine |
| | | | | | kinases |
| | . | Liver | . | . | . |
| | . | n | . | . | . |
| TSBTx3837 | . | n | . | | . |

### Example 5: Base Editing of Complement Factor B (CFB) in Non-Human Primates

Experiments were undertaken to demonstrate knock-out of complement factor B (CFB) in non-human primates (cynomolgus monkeys) using base editing. The non-human primates were administered a single-dose intravenous infusion containing lipid nanoparticles containing the base editor systems of Table 18, which contained mRNA encoding a base editor together with a guide RNA molecule.

TABLE 18
Base editor systems used to edit CFB in non-human primates. “ABE9.52”
refers to a base editor containing the following adenosine deaminase domain:
TadA*8.20 with the amino acid alterations V82T, Y147T, and Q154S.

| Grp | | | | | | | Dose | | |
|---|---|---|---|---|---|---|---|---|---|
| 1 | Target | Editor | mRNA | gRNA | gRNA Lot | LNP | (mpk) | n | TD |
| 1 | ALAS | ABE8.8 | mRNABTx4582- | gRNA359 | gRNA359- | Yes | 1.5 | 3 | D15 |
| | 1 | | 001 | | 1241 | | | | Liver |
| 5 | CFB 1 | ABE9.52 | mRNABTx4712- | gRNA2445 | gRNA2445- | | 1.5 | 3 | Biopsy, |
| | | | 001 | | 6731 | | | | TakedowN |
| 6 | CFB 2 | ABE8.20 | mRNABT×4839- | gRNA5856 | gRNA5856- | | 1.5 | 3 | Day 60 |
| | | | 001 | | 6732 | | | | |
| 7 | CFB 1 | ABE9.52 | mRNABTx4712- | gRNA2445 | gRNA2445- | | 2.5 | 3 | |
| | | | 001 | | 6731 | | | | |

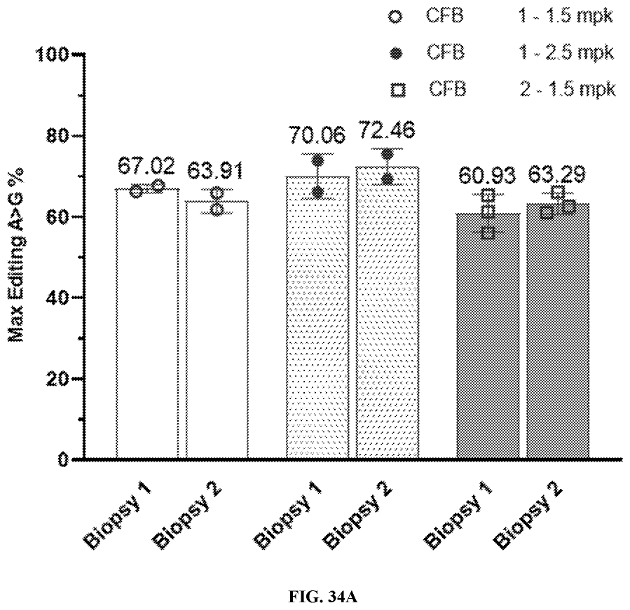

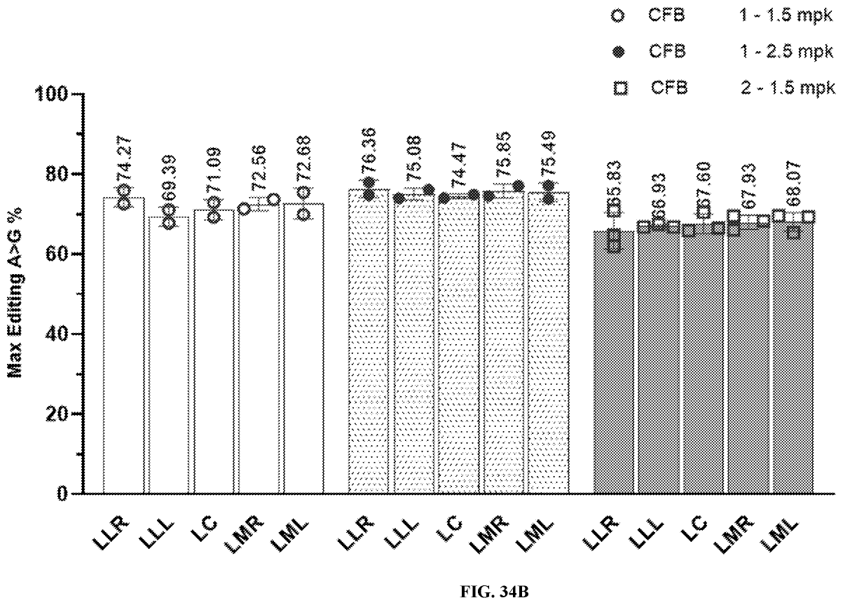

On Day 15 following the infusions, liver biopsies were collected and on-target editing rates of about 65.5% and 71.3% were detected in liver biopsies collected from non-human primates administered base editor systems corresponding to Groups 5 and 7 of Table 18, respectively (FIG. 34A). The doses administered to the non-human primates were measured in terms of total gRNA. On Day 60 liver necropsies showed base editing rates of about 72.0% and 75.5% in non-human primates administered base editor systems corresponding to Groups 5 and 7 of Table 18, respectively (FIG. 34B and Table 19). Non-human primates administered the base editor systems at the doses of 1.5 mg/kg or 2.5 mg/kg showed near-saturating or saturating on-target editing of the CFB gene in the liver (FIGS. 34A and 34B).

TABLE 19
Data relating to base editing of CFB in non-human
primates using the base editor systems of Table 18.

| | | % Reduction from Baseline- Avg Day 57 | | | |
|---|---|---|---|---|---|
| | Necropsy | Factor B | | | |
| | % Base | Protein Level | Bb | AH50 | CH50 |
| Treatment | Editing | (FBL) (Plasma) | (Plasma) | (Serum) | (Serum) |
| Control | 54.0% | −3 | −26 | −18 | −12.5 |
| (ALAS) | | | | | |
| CFB 1- | 72.0% | −93 | −83.5 | −84 | 6.5 |
| 1.5 mg/kg | | | | | |
| CFB 1- | 75.5% | −93 | −83.5 | −93 | −9 |
| 2.5 mg/kg | | | | | |
| CFB 2- | 67.3% | −94 | −86.3 | −83 | −6.5 |
| 1.5 mg/kg | | | | | |

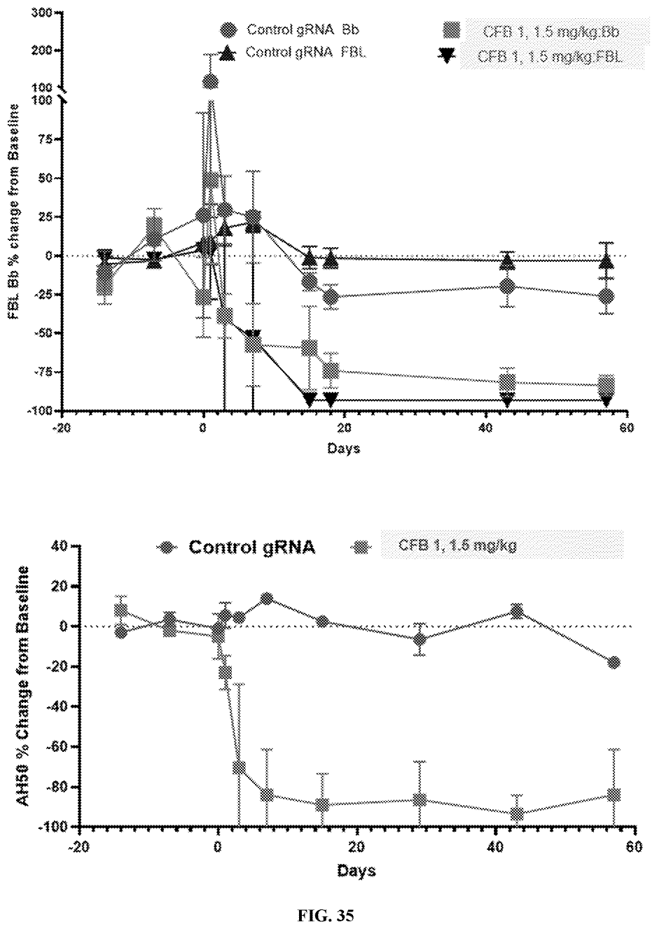

The non-human primates administered the base editor systems of Table 19 showed reductions total CFB protein levels (FBL) (FIG. 35 and Table 19). The non-human primates showed a reduction in hemolytic assay measuring alternative pathway (AH-50) of up to 93% (see Table 19). A reduction in total CFB protein of between about 93% and 94% was observed in the non-human primates administered the base editor systems together with a reduction in Bb protein between about 83% and 87% (see Table 19).

### Example 6: Base Editing of Human Complement Factor B (hCFB) in Transgenic Mice

Experiments were undertaken to demonstrate knock-out of complement factor B (CFB) in transgenic mice using base editing. The mice (Mus musculus) were B-hCFB mice (C57BL/6N-Cfb^{tm1(CFB)}/Bcgen) obtained from Jiangsu Biocytogen Co. Ltd., China, where exons 1~18 of the mouse Cfb gene encoding the full-length CFB protein were replaced by human CFB exons 1~18. The mice were either homozygous or heterozygous for the human CFB exons.

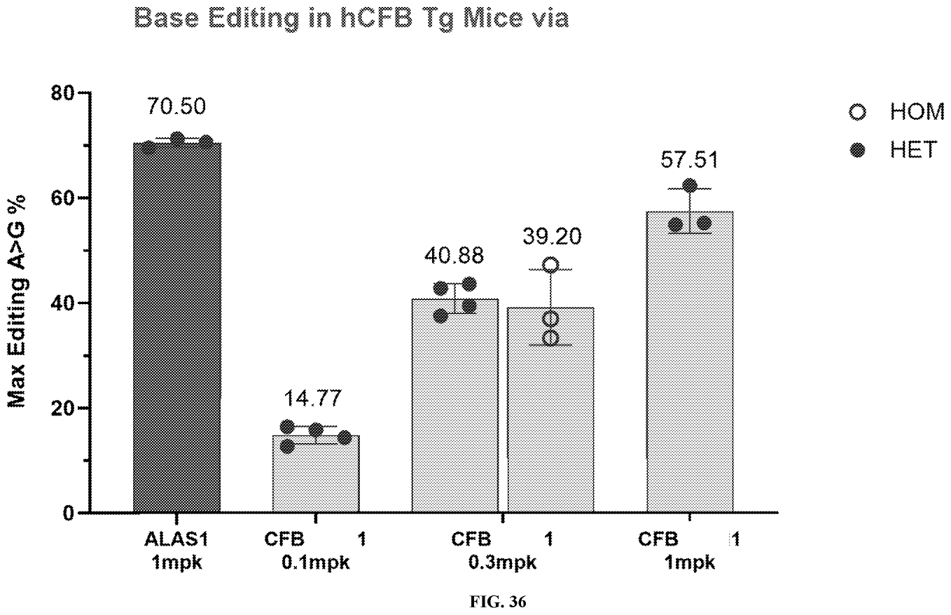

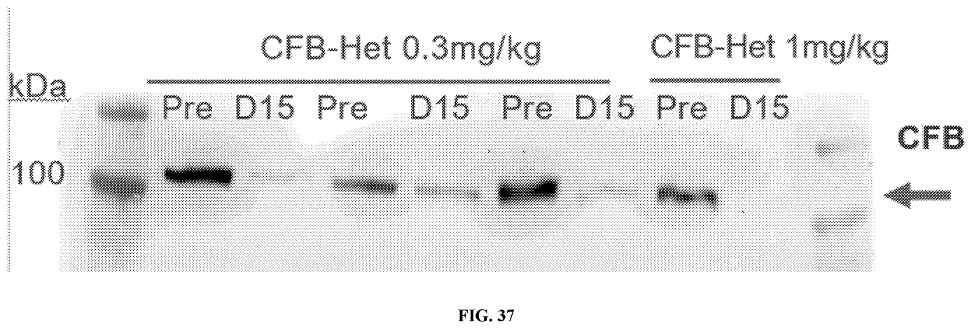

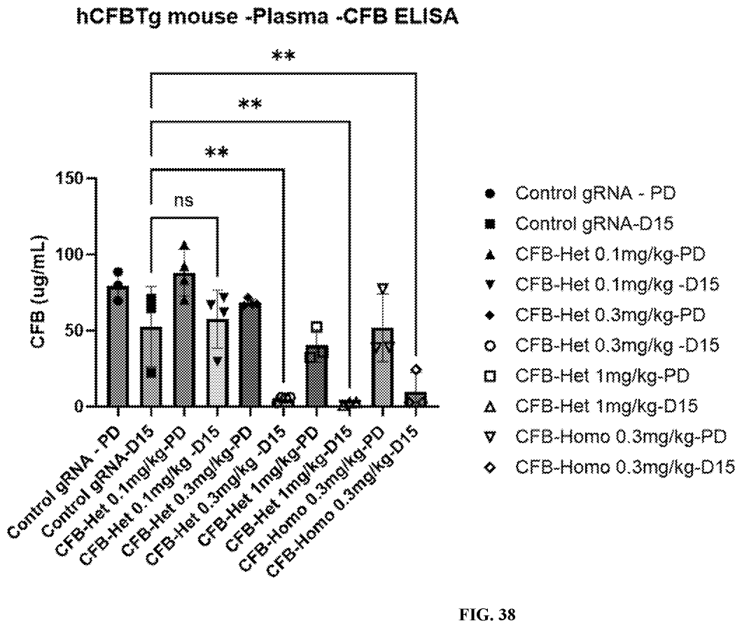

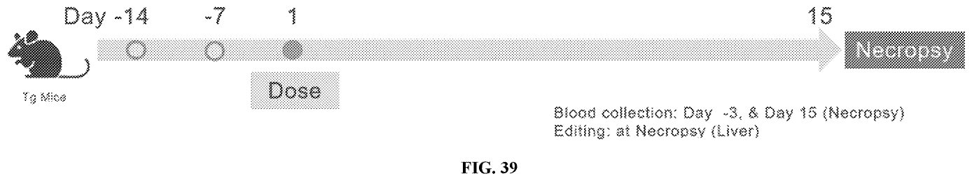

In one experiment, the mice were administered lipid nanoparticles containing the base editor systems described in Tables 20 and 21, which contained mRNA encoding a base editor together with a guide RNA. As a control, the mice were administered lipid nanoparticles containing a base editor system containing a total of 1 mg/kg (mpk) of gRNA targeting ALAS1 for editing together with mRNA encoding a base editor (Group 1 in Table 21). The doses administered to the mice were measured in terms of total gRNA. At day 15 following administration of the lipid nanoparticles, liver necropsies revealed dose-dependent levels of base editing were observed in mice dosed with 0.1, 0.3, or 1 mpk of the base editor systems targeting the hCFB polynucleotide for base editing (Groups 2 to 5 of Table 21) (FIG. 36). The mice of Group 1 of Table 21 appeared to show saturated levels of liver editing of the ALAS1 gene (FIG. 9). Also, plasma levels of hCFB in the mice administered the base editor systems targeting the hCFB polynucleotide for base editing were reduced relative to pre-dosing levels, as measured using immunoblotting and ELISA (FIGS. 37 and 38). An Abcam Elisa Kit (Human Factor B ELISA Kit (ab137973)) was used to measure serum protein levels. Serum hCFB was undetectable in mice administered a 1 mg/kg does of the base editor systems targeting the hCFB polynucleotide for base editing. Mice administered 0.3 mg/kg or 1 mg/kg of the base editor systems (i.e., Formulation 1 of Table 21) showed group mean reductions in CFB protein levels of about 82% and 85%, respectively. Homozygous (HOM) mice treated with 0.3 mg/kg of Formulation 1 of Table 21 (CFB Lead 1 NLS) achieved an average reduction in Factor B protein levels of about 77%. No difference in base editing as observed between mice homozygous or heterozygous for the hCFB exons.

TABLE 20
Base editor systems used to edit CFB in transgenic mice, where the terms “NLS”, “End-
Mod” and “Lit Mod 1 / HMO1” refer to modifications of the gRNA molecules.
“ABE9.52” refers to a base editor containing the following adenosine
deaminase domain: TadA*8.20 with the amino acid alterations V82T, Y147T, and Q154S.

| | | | Corresponding gRNA # | |
|---|---|---|---|---|
| | | | for targeting cyno non- | |
| | Alternative | gRNA # for | human primates (NHP) | Base |
| Name | Name | human gene | gene | Editor |
| CFB 1 (NLS) | Formulation 1 | gRNA2445 | gRNA2445 | ABE9.52 |
| CFB 1 (End- Mod) | Formulation 2 | gRNA1193 | gRNA1193 | ABE9.52 |
| CFB 2 (Lit Mod 1/ | Formulation 3 | gRNA2451 | gRNA5856 | ABE8.8 |
| HMO1) | | | | |

TABLE 21
Design of validation study carried out in transgenic mice. “ABE9.52” refers to
a base editor containing the following adenosine deaminase domain: TadA*8.20 with
the amino acid alterations V82T, Y147T, and Q154S.

| | | Base | | | | | | | | | | |
|---|---|---|---|---|---|---|---|---|---|---|---|---|
| | | Editor | | | | | | | | | | |
| | | System | mRNA | | mRNA | | gRNA | | Dose | | Animal | |
| Grp | Target | Name | Template | Editor | Number | sgRNA | Lot | LNP | (mg/kg) | n | Genotype | TD |
| 1 | ALAS1 | | pMSP546 | ABE8.8 | mRNA4582 | sgRNA_359 | sgRNA_3 | Yes | 1 | 3 | 3HET | 14 |
| | | | | | | (sgRNA_088 | 59-1241 | | | | | |
| | | | | | | modified | | | | | | |
| | | | | | | to have | | | | | | |
| | | | | | | an HMO1 | | | | | | |
| | | | | | | nucleotide | | | | | | |
| | | | | | | modification | | | | | | |
| | | | | | | scheme) | | | | | | |
| 2 | CFB | Formulationl | pMSP1197 | ABE9.52 | mRNA4712 | gRNA2445 | gRNA24 | | 0.1 | 4 | HET | 14 |
| 3 | | | | | | | 45-6731 | | 0.3 | 4 | HET | 14 |
| 4 | | | | | | | | | 1 | 3 | HET | 14 |
| 5 | | | | | | | | | 0.3 | 3 | HOM | 14 |

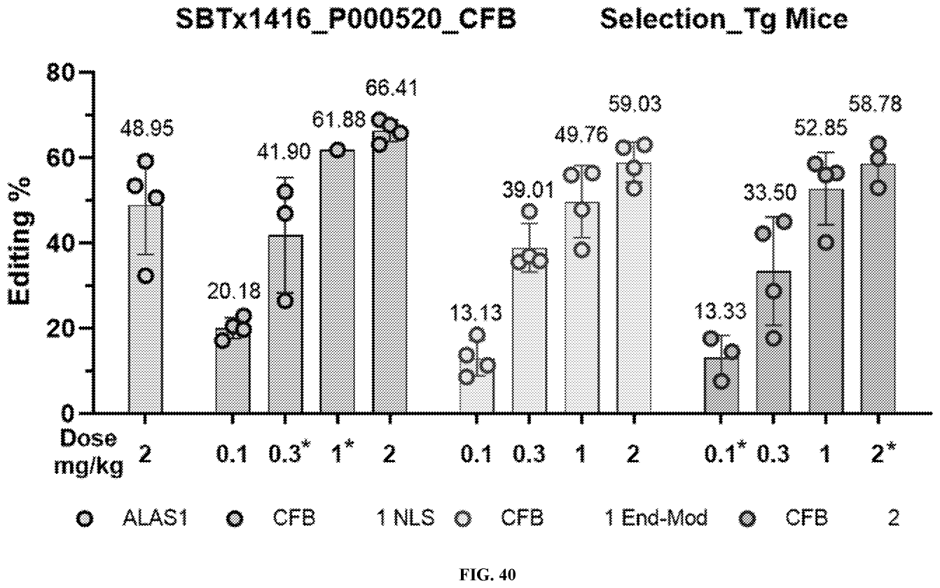

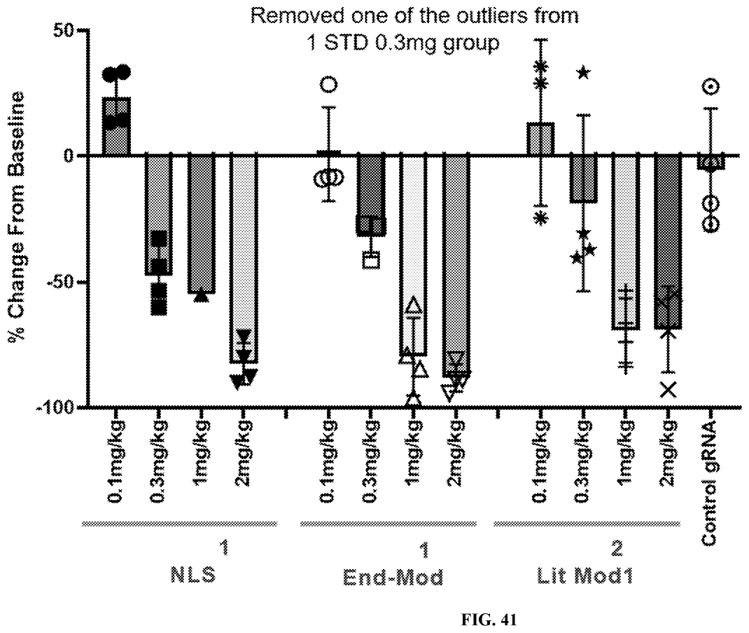

An experiment was undertaken to measure the effect of base editor system dose levels on base editing rates and reductions in hCFB protein levels in the mice. The transgenic mice were administered a single-dose intravenous injection of lipid nanoparticles containing the base editor systems listed in Table 22 (i.e., Groups 1 to 13), where each base editor system contained a guide RNA molecule and mRNA encoding a base editor. The doses (i.e., 0.1, 0.3, or 2 mg/kg) of the base editor systems administered to the mice were measured in terms of total gRNA. Dose-dependent on-target base editing levels in the liver and reductions in plasma hCFB protein levels were observed in the mice administered all 3 formulations described in Tables 21 and 22, as measured at day 14 following administration of the base editor systems (FIGS. 40 and 41). A dose of 0.3 mg/kg of each formulation was associated with high on-target base editing efficiencies ranging from about 33% to about 42% in the livers of the transgenic mice. For mice treated with Formulation 1 of Table 22 at doses of 0.1, 0.3, 1, and 2 mg/kg, the average reduction rates of CFB protein were 48%, 55%, and 83% relative to baseline, respectively. In the case of mice given Formulation 2 of Table 22 at the same dosage levels, the mean reductions in CFB protein were 32%, 80%, and 88% relative to baseline, respectively. Mice administered Formulation 3 of Table 22 at 0.1, 0.3, 1, and 2 mg/kg showed mean CFB protein reductions of 19%, 69%, and 69% relative to baseline, respectively.

TABLE 22
Base editor systems used to edit CFB in transgenic mice. “ABE9.52” refers to
a base editor containing the following adenosine deaminase domain: TadA*8.20 with the amino
acid alterations V82T, Y147T, and Q154S.

| | Target | | | n | | Dose | Alternative |
|---|---|---|---|---|---|---|---|
| Group | Gene | gRNA/BE | | Dosed | TD | (mg/kg) | Name |
| 1 | ALAS1 | HM01 + | sgRNA_359-1241 | 4 | 4 | 1 | N/A |
| | | ABE8.8 | (sgRNA_088 | | | | |
| | | | modified to have an | | | | |
| | | | HMO1 nucleotide | | | | |
| | | | modification | | | | |
| | | | scheme)/mRNA3171 | | | | |
| 2 | CFB | 1 | 1 | 4 | 4 | 0.1 | Formulation |
| 3* | TSBTx3826 | NLS + | gRNA2445-8043 + | 4 | 3 | 0.3 | 1 |
| 4** | | ABE9.52 | mRNA3969 | 4 | 1 | 1 | |
| 5 | | | | 4 | 4 | 2 | |
| 6 | CFB | 1 | 1 | 4 | 4 | 0.1 | Formulation |
| 7 | TSBTx3826 | End- | gRNA1193-1862 + | 4 | 4 | 0.3 | 2 |
| 8 | | mod + | mRNA3969 | 4 | 4 | 1 | |
| 9 | | ABE9.52 | | 4 | 4 | 2 | |
| 10* | CFB | 2 | 2 | 4 | 3 | 0.1 | Formulation |
| 11 | TSBTx3837 | Lit | gRNA2451-8042 + | 4 | 4 | 0.3 | 3 |
| 12 | | Mod1/ | mRNA3171 | 4 | 4 | 1 | |
| 13* | | HMO1 + | | 4 | 3 | 2 | |
| | | ABE8.8 | | | | | |

### OTHER EMBODIMENTS

From the foregoing description, it will be apparent that variations and modifications may be made to the aspects or embodiments described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

The disclosure may be related to International Patent Application No. PCT/US2023/068543, filed Jun. 15, 2023, the entirety of which is incorporated herein by reference for all purposes.

What is claimed:

1. A method of treating a disease or disorder associated with inappropriate activation of the complement system in a subject in need thereof, the method comprising altering a nucleobase of a complement factor B (CFB) polynucleotide in the subject by administering to the subject one or more guide polynucleotides, or one or more polynucleotides encoding the one or more guide polynucleotides, and a base editor comprising a nucleic acid programmable DNA binding protein (napDNAbp) domain and a deaminase domain, or one or more polynucleotides encoding the base editor, wherein
(a) said one or more guide polynucleotides targets said base editor to effect an alteration of a nucleobase of the CFB polynucleotide that:
i. disrupts a splice site in the CFB polynucleotide,
ii. alters a start codon in the CFB polynucleotide,
iii. alters a TATA box in the CFB polynucleotide,
iv. introduces a new stop codon in the CFB polynucleotide, and/or
v. alters a nucleobase in a codon encoding an amino acid residue within a region of the CFB polypeptide encoded by the CFB polynucleotide selected from the group consisting of: serine protease (SP) active site, Mg²⁺ binding loop, cleavage site, salt bridge, and oxyanion-hole;
(b) the deaminase domain comprises a TadA variant (TadA*) comprising an amino acid sequence having at least 90% sequence identity to the following TadA*7.10 amino acid sequence: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), or a fragment thereof lacking only the N-terminal methionine, wherein the TadA* further comprises a combination of amino acid alterations compared to the TadA*7.10 amino acid sequence selected from the group consisting of:
i. I76Y, V82T, Y123H, Y147T, and Q154S,
ii. Y123H, Y147R, and Q154R,
iii. 176Y, Y133H, Y147R, and Q154R,
iv. V82S, and Q164R,
v. I76Y, V82S, Y123H, Y147R, and Q154R, and
vi. I76Y, V82T, Y123H, Y147R, and Q154;
(c) the one or more guide polynucleotides comprises a nucleic acid sequence selected from CCUCAGAUGUCUAUGUGUUU (SEQ ID NO: 1524), UGCUUACAAUGACUGAGAUCU (SEQ ID NO: 1535), UUGCUCCCCAUGGCGUUGGA (SEQ ID NO: 3476), CCCCAUGGCGUUGGAAGGCA (SEQ ID NO: 3443), and UGCUCCCCAUGGCGUUGGAA (SEQ ID NO: 3467) and/or comprising at least 10-23 contiguous nucleotides of a spacer nucleic acid sequence listed in any one of Tables 2A to 2H; and/or
(d) said one or more guide polynucleotides targets said base editor to effect an alteration of a nucleobase in one or more codons encoding an amino acid residue selected from the group consisting of amino acid residue 1, 171, 175, 176, 177, 202, 203, 229, 230, 231, 232, 233, 254, 255, 256, 257, 258, 259, 260, 275, 276, 277, 278, 279, 280, 281, 351, 353, 354, 389, 470, 471, 472, 525, 526, 529, 574, 575, 576, 696, 697, and 699 relative to the following reference sequence:
Complement factor B amino acid sequence MGSNLSPQLCLMPFILGLLSGGVTTTPWSLAQPQGSCSLEGVEIKGGSFRLLQEGQALEYVC PSGFYPYPVQTRTCRSTGSWSTLKTQDQKTVRKAECRAIHCPRPHDFENGEYWPRSPYYNVS DEISFHCYDGYTLRGSANRTCQVNGRWSGQTAICDNGAGYCSNPGIPIGTRKVGSQYRLEDS VTYHCSRGLTLRGSQRRTCQEGGSWSGTEPSCQDSFMYDTPQEVAEAFLSSLTETIEGVDAE DGHGPGEQQKRKIVLDPSGSMNIYLVLDGSDSIGASNFTGAKKCLVNLIEKVASYGVKPRYG LVTYATYPKIWVKVSEADSSNADWVTKQLNEINYEDHKLKSGTNTKKALQAVYSMMSWPDDV PPEGWNRTRHVIILMTDGLHNMGGDPITVIDEIRDLLYIGKDRKNPREDYLDVYVFGVGPLV NQVNINALASKKDNEQHVFKVKDMENLEDVFYQMIDESQSLSLCGMVWEHRKGTDYHKQPWQ AKISVIRPSKGHESCMGAVVSEYFVLTAAHCFTVDDKEHSIKVSVGGEKRDLEIEVVLFHPN YNINGKKEAGIPEFYDYDVALIKLKNKLKYGQTIRPICLPCTEGTTRALRLPPTTTCQQQKE ELLPAQDIKALFVSEEEKKLTRKEVYIKNGDKKGSCERDAQYAPGYDKVKDISEVVTPRFLC TGGVSPYADPNTCRGDSGGPLIVHKRSRFIQVGVISWGVVDVCKNQKROKQVPAHARDFHIN LFQVLPWLKEKLQDEDLGFL (SEQ ID NO: 426), or a corresponding position in another CFB polypeptide sequence;
thereby altering the nucleobase of the CFB polynucleotide.

2. A method of treating a disease or disorder associated with inappropriate activation of the complement system in a subject in need thereof, the method comprising altering a nucleobase of a complement factor B (CFB) polynucleotide in the subject by administering to the subject one or more guide polynucleotides, or one or more polynucleotides encoding the guide polynucleotides, and a base editor comprising a nucleic acid programmable DNA binding protein (napDNAbp) domain and a deaminase domain, or one or more polynucleotides encoding the base editor, wherein
(a) the deaminase domain comprises a cytidine deaminase or a TadA variant (TadA*) comprising an amino acid sequence having at least 90% sequence identity to the following TadA*7.10 amino acid sequence: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), wherein the TadA* further comprises a combination of amino acid alterations compared to the TadA*7.10 amino acid sequence selected from the group consisting of:
i. I76Y, V82T, Y123H, Y147T, and Q154S,
ii. Y123H, Y147R, and Q154R,
iii. I76Y, Y133H, Y147R, and Q154R,
iv. V82S, and Q164R,
v. I76Y, V82S, Y123H, Y147R, and Q154R, and
vi. I76Y, V82T, Y123H, Y147R, and Q154R; and
(b) the one or more guide polynucleotides comprise a spacer comprising a nucleotide sequence selected from the group consisting of: CCUCAGAUGUCUAUGUGUUU (SEQ ID NO: 1524; TSBTx3826), AGGUGAUUCUGGCGGCCCCU (SEQ ID NO: 1719; gRNA1536), CGCCAGAAUCACCUGCAAGG (SEQ ID NO: 1715; gRNA1532), CUAUGACGUUGCCCUGAUCA (SEQ ID NO: 1723; gRNA1540), UGCUCCCCAUGGCGUUGGAA (SEQ ID NO: 3467; gRNA3657), UUGCUCCCCAUGGCGUUGGA (SEQ ID NO: 3476; gRNA3658), CCCCAUGGCGUUGGAAGGCA (SEQ ID NO: 3443; gRNA3660), GCUUACAAUGACUGAGAUCU (SEQ ID NO: 1534; TSBTx3837), UGCUUACAAUGACUGAGAUCU (SEQ ID NO: 1535; TSBTx3837), and UCUCACCUCUGCAAGUAUUG (SEQ ID NO: 1529; TSBTx3835);
thereby treating the disease or disorder associated with inappropriate activation of the complement system in the subject.

3. The method of claim 1, wherein the one or more guide polynucleotides target said base editor to effect an alteration of the nucleobase of the CFB polynucleotide that disrupts a splice site in the CFB polynucleotide.

4. The method of claim 1, wherein the napDNAbp is a nickase.

5. The method of claim 1, wherein the napDNAbp binds a protospacer adjacent motif (PAM) selected from the group consisting of NGA, NGC, NGG, and NNNRRT, wherein “N” is any nucleotide and “R” is A or G.

6. The method of claim 5, wherein the napDNAbp is a Cas9 polypeptide.

7. The method of claim 1, wherein the one or more guide polynucleotides comprises a modified nucleotide.

8. The method of claim 7, wherein the one or more guide polynucleotides comprises a sequence selected from the group consisting of:
End-mod SpCas9 guide polynucleotide mNsmNsmNsNNNNNNNNNNNNNNNNNGUUUUAGAGCUAGAAAUAGCAAGUUAAAAUAAGGCU AGUCCGUUAUCAACUUGAAAAAGUGGCACCGAGUCGGUGCUsmUsmUsmU (SEQ ID NO: 440);
End-mod SaCas9 guide polynucleotide mNsmNsmNsNNNNNNNNNNNNNNNNNNGUUUUAGUACUCUGUAAUGAAAAUUACAGAAUCUA CUAAAACAAGGCAAAAUGCCGUGUUUAUCUCGUCAACUUGUUGGCGAGAUsmUsmUsmU (SEQ ID NO: 441);
HM01: mNsmNsmNsNNNNNNNNNNNNNNNNNGUUUUAGAmGmCmUmAmGmAmAmAmUmAmGmCAAGU UAAAAUAAGGCUAGUCCGUUAUCAmAmCmUmUmGmAmAmAmAmAmGmUmGmGmCmAmCmCmG mAmGmUmCmGmGmUmGmCmUsmUsmUsmU (SEQ ID NO: 440);
HM07: mNsmNsmNsmNmNmNmNmNmNmNNNNNNNNNNNmGUUUUAGmAmGmCmUmAmGmAmAmAmUm AmGmCmAmAGUUmAAmAAmUAmAmGmGmCmUmAGUmCmCGUUAmUmCAAmCmUmUmGmAmAm AmAmAmGmUmGGmCmAmCmCmGmAmGmUmCmGmGmUmGmCmUsmUsmUsmU (SEQ ID NO: 440);
NLS (bpsv40): mNsmNsmNsNNNNNNNNNNNNNNNNNGUUUUAGAGCUAGAAAUAGCAAGUUAAAAUAAGGCU AGUCCGUUAUCAACUUGAAAAAGUGGCACCGAGUCGGUGCmUsmUsmUsmU-NHC6-CrossL-ac-CKRTADGSEFESPKKKRKV (SEQ ID NOs: 440 and 446);
LONGEST: mNsmNsmNsNNNNNNNNNNNNNNNNNGUUUUAGAmGmCmCmGmGmCmGmGmAmAmAmCmGmC mCmGmGmCAAGUUAAAAUAAGGCUAGUCCGUUAUCAmAmCmUmUmGmAmAmAmAmAmGUGmG mCmAmCmCmGmAmGmUmCmGmGmUmGmCmUsmUsmUsmU (SEQ ID NO: 445);
NLS+LONGEST: mNsmNsmNsNNNNNNNNNNNNNNNNNGUUUUAGAmGmCmCmGmGmCmGmGmAmAmAmCmGmC mCmGmGmCAAGUUAAAAUAAGGCUAGUCCGUUAUCAmAmCmUmUmGmAmAmAmAmAmGmUmG mGmCmAmCmCmGmAmGmUmCmGmGmUmGmCmUsmUsmUsmU-NHC5-CrossL-CKRTADGSEFESPKKKRKV (SEQ ID NOs: 445 and 446); and
LONGEST+GOLD: mNsmNsmNsNNNNNNNNNNNNNNNNNGUUUUAGAmGmCmCmGmGmCmGmGmAmAmAmCmGmC mCmGmGmCAAGUUAAAAUAAGGCUAGUCCGUUAmUmCAAmCmUmUGGACUUCGGUCCmAmAm GUGGmCmAmCmCmGmAmGmUmCmGmGmUmGmCmUsmUsmUsmU (SEQ ID NO: 447);
wherein “N” represents any nucleotide, “mN” indicates a 2′-OMe modification of the nucleotide “N”, and “Ns” indicates that the nucleotide “N” is linked to the following nucleotide by a phosphorothioate (PS), and wherein the number of N nucleotides is between 15 and 25.

9. The method of claim 1, wherein the nucleobase alteration results in disruption of Mg²⁺ binding to the CFB polypeptide encoded by the CFB polynucleotide or results in a reduction or elimination of serine protease activity of the CFB polypeptide encoded by the CFB polynucleotide.

10. The method of claim 1, wherein the nucleobase alteration eliminates a salt bridge of the CFB polypeptide encoded by the CFB polynucleotide.

11. The method of claim 1, wherein the base editor effects a nucleobase alteration to the CFB polynucleotide that reduces cleavage of the CFB polypeptide encoded by the CFB polynucleotide by a factor D polypeptide.

12. The method of claim 1, wherein the deaminase domain is an adenosine deaminase comprising the TadA*7.10 amino acid sequence further comprising a combination of amino acid alterations selected from the group consisting of:
i. I76Y, V82T, Y123H, Y147T, and Q154S,
ii. Y123H, Y147R, and Q154R,
iii. I76Y, Y133H, Y147R, and Q154R,
iv. V82S, and Q164R,
v. I76Y, V82S, Y123H, Y147R, and Q154R, and
vi. I76Y, V82T, Y123H, Y147R, and Q154R.

13. The method of claim 1, wherein said one or more guide polynucleotides target said base editor to effect an alteration of a nucleobase in a codon encoding an amino acid residue selected from the group consisting of M1, P171, V177, R203, E232, E255, K258, R259, K260, D276, S278, S280, T353, D389, E471, H526, Y575, D576, G697, and S699 relative to the following reference sequence:
Complement factor B amino acid sequence MGSNLSPQLCLMPFILGLLSGGVTTTPWSLAQPQGSCSLEGVEIKGGSFRLLQEGQALEYVC PSGFYPYPVQTRTCRSTGSWSTLKTQDQKTVRKAECRAIHCPRPHDFENGEYWPRSPYYNVS DEISFHCYDGYTLRGSANRTCQVNGRWSGQTAICDNGAGYCSNPGIPIGTRKVGSQYRLEDS VTYHCSRGLTLRGSQRRTCQEGGSWSGTEPSCQDSFMYDTPQEVAEAFLSSLTETIEGVDAE DGHGPGEQQKRKIVLDPSGSMNIYLVLDGSDSIGASNFTGAKKCLVNLIEKVASYGVKPRYG LVTYATYPKIWVKVSEADSSNADWVTKQLNEINYEDHKLKSGTNTKKALQAVYSMMSWPDDV PPEGWNRTRHVI ILMTDGLHNMGGDPITVIDEIRDLLYIGKDRKNPREDYLDVYVFGVGPLV NQVNINALASKKDNEQHVFKVKDMENLEDVFYQMIDESQSLSLCGMVWEHRKGTDYHKQPWQ AKISVIRPSKGHESCMGAVVSEYFVLTAAHCFTVDDKEHSIKVSVGGEKRDLEIEVVLFHPN YNINGKKEAGIPEFYDYDVALIKLKNKLKYGOTIRPICLPCTEGTTRALRLPPTTTCQQQKE ELLPAQDIKALFVSEEEKKLTRKEVYIKNGDKKGSCERDAQYAPGYDKVKDISEVVTPRFLC TGGVSPYADPNTCRGDSGGPLIVHKRSRFIQVGVISWGVVDVCKNQKROKOVPAHARDFHIN LFQVLPWLKEKLQDEDLGFL (SEQ ID NO: 426).

14. The method of claim 1, wherein CFB activity, protein concentration, and/or mRNA concentration is reduced by at least about 15% as compared to a control subject without the alteration.

15. The method of claim 1, wherein the inappropriate activation of the complement system is associated with increased levels of one or more of inflammation, the presence of autoantibodies, neural degeneration, and microthrombosis.

16. The method of claim 1, wherein the inappropriate activation of the complement system is associated with damage to the central nervous system (CNS), the eyes, the gastrointestinal system, the pulmonary system, the musculoskeletal system, the circulatory system, the integumentary system, blood cells, thyroid, kidney, joints, gastrointestinal system, or transplanted organs.

17. The method of claim 1, wherein the disease or disorder is selected from the group consisting of acute antibody-mediated rejection, age-related macular degeneration, allergic bronchopulmonary aspergillosis, allergic neuritis, allergic rhinitis, Alzheimer's disease, amyotrophic lateral sclerosis (ALS), anaphylaxis, scleritis, atopic dermatitis, atypical hemolytic syndrome (aHUS), autoimmune hemolytic anemia, Bechet's disease, bronchiolitis, IC-MPGN/C3 glomerulopathy, central nervous system (CNS) inflammatory disorders, choroidal neovascularization (CNV), choroiditis, chronic allograft vasculopathy, chronic hepatitis, chronic muscle inflammation, chronic pain, chronic pancreatitis, chronic urticaria, Churg-Strauss syndrome, conjunctivitis, cyclitis, demyelinating disease, dermatitis, dermatomyositis, diabetic retinopathy, encephalitis, eosinophilic pneumonia, geographic atrophy, giant cell arteritis, glaucoma, glomerulonephritis, graft or transplant rejection or failure, HELLP syndrome, Henoch-Schonlein purpura, hypersensitivity pneumonitis, idiopathic pulmonary fibrosis (IPF), IgA nephropathy (IgAN), inflammatory bowel diseases, inflammatory joint conditions, inflammatory skin diseases, infusion reactions, interstitial pneumonia, iridocyclitis, iritis, ischemia/reperfusion injury, Kawasaki disease, keratitis, lupus nephritis, membranoproliferative glomerulonephritis (MPGN), meningitis, microscopic polyangiitis, myasthenia gravis, myocarditis, nasal polyposis, neuromyelitis optica, neuropathic pain, ocular inflammation, osteoarthritis, pancreatitis, panniculitis, paroxysmal nocturnal hemoglobinuria (PNH), pars planitis, pemphigoid, pemphigus, polyarteritis nodosa, polymyositis, primary membranous nephropathy, proliferative vitreoretinopathy, proteinuria, psoriasis, pulmonary fibrosis, renal disease, respiratory distress syndrome, retinal neovascularization (RNV), retinopathy of prematurity, rheumatoid arthritis (RA), rhinosinusitis, sarcoid, sarcoidosis, scleritis, scleroderma, sclerodermatomyositis, sclerosis, Sjögren syndrome, systemic lupus erythematosus, systemic scleroderma, Takayasu's arteritis, Tautopathies, thyroiditis, thyroidoisis, ulcerative colitis, uveitis, vasculitis, and Wegener's granulomatosis.

18. The method of claim 1, wherein the administration is local administration to an eye, to spinal fluid, or to the liver.

19. The method of claim 1, wherein the CFB polynucleotide is contacted with two or more guide polynucleotides, and wherein each guide polynucleotide binds a different location within the CFB polynucleotide.

20. The method of claim 1, wherein the subject is a mammal.
